(19) United States
(12) Patent Application Publication
(10) Pub. No.: US 2023/0159510 A1
(43) Pub. Date: May 25, 2023

(54) NOVEL PRMT5 INHIBITORS

(71) Applicant: AMGEN INC., Thousand Oaks, CA (US)

(72) Inventors: Jennifer Rebecca ALLEN, Newbury Park, CA (US); Albert AMEGADZIE, Westlake Village, CA (US); Diane Jennifer BEYLKIN, Thousand Oaks, CA (US); Shon BOOKER, Sherman Oaks, CA (US); Matthew Paul BOURBEAU, Woodland Hills, CA (US); John R. BUTLER, Thousand Oaks, CA (US); Michael J. FROHN, Thousand Oaks, CA (US); Sanne Ormholt Schroder GLAD, Ballerup (DK); Birgitte Weinreich HUSEMOEN, Copenhagen (DK); Matthew R. KALLER, Ventura, CA (US); Todd J. KOHN, Thousand Oaks, CA (US); Brian Alan LANMAN, Woodland Hills, CA (US); Kexue LI, Newbury Park, CA (US); Qingyian LIU, Camarillo, CA (US); Patricia LOPEZ, Woodland Hills, CA (US); Vu Van MA, Oak Park, CA (US); Francesco MANONI, Newbury Park, CA (US); Jose MEDINA, Thousand Oaks, CA (US); Ana Elena MINATTI, Santa Monica, CA (US); Jorge PEIRO CADAHIA, Madrid (ES); Liping PETTUS, Thousand Oaks, CA (US); Alexander J. PICKRELL, Westlake Village, CA (US); Ian SARVARY, Klagshamn (SE); Nuria A. TAMAYO, Newbury Park, CA (US); Mikkel VESTERGAARD, Greve (DK)

(73) Assignee: AMGEN INC., Thousand Oaks, CA (US)

(21) Appl. No.: 17/799,077

(22) PCT Filed: Feb. 11, 2021
(86) PCT No.: PCT/US2021/017682

§ 371 Date: Aug. 11, 2022

Related U.S. Application Data
(60) Provisional application No. 62/975,258, filed on Feb. 12, 2020.

(51) Int. Cl. C07D 413/14; C07D 471/04; C07D 401/14; C07D 401/12; C07D 405/14

(52) U.S. Cl. CPC C07D 413/14; C07D 401/12; C07D 401/14; C07D 405/14; C07D 471/04

(57) ABSTRACT

Described herein are compounds of Formula I and pharmaceutically acceptable salt thereof, stereoisomers, tautomers as well as pharmaceutical compositions thereof. Compounds of the present invention are useful for inhibiting PRMT5 activity and may have use in treating proliferative, metabolic and blood disorders. Compounds of Formula I have the following structure: (I)

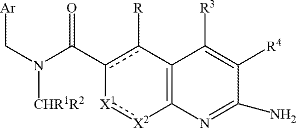

### BACKGROUND OF THE INVENTION

Epigenetic regulation of gene expression is an important biological determinant of protein production and cellular differentiation and plays a significant pathogenic role in a number of human diseases.

Epigenetic regulation involves heritable modification of genetic material without changing its nucleotide sequence. Typically, epigenetic regulation is mediated by selective and reversible modification (e.g., methylation) of DNA and proteins (e.g., histones) that control the conformational transition between transcriptionally active and inactive states of chromatin. These covalent modifications can be controlled by enzymes such as methyltransferases (e.g., PRMT5), many of which are associated with specific genetic alterations that can cause human disease. PRMT5 plays a role in diseases such as proliferative disorders, metabolic disorders, and blood disorders.

The homozygous deletion of tumor suppressor genes is a key driver of cancer, frequently resulting in the collateral loss of passenger genes located in close genomic proximity to the tumor suppressor. Deletion of these passenger genes can create therapeutically tractable vulnerabilities that are specific to tumor cells. Homozygous deletion of the chromosome 9p21 locus, which harbors the well-known tumor suppressor CDKN2A (cyclin dependent kinase inhibitor 2A), occurs in 15% of all tumors and frequently includes the passenger gene MTAP (methylthioadenosine phosphorylase), a key enzyme in the methionine and adenine salvage pathways. Deletion of MTAP results in accumulation of its substrate, methylthioadenosine (MTA). MTA shares close structural similarity to S-adenosylmethionine (SAM), the substrate methyl donor for the type II methyltransferase PRMT5. Elevated MTA levels, driven by loss of MTAP, selectively compete with SAM for binding to PRMT5, placing the methyltransferase in a hypomorphic state, vulnerable to further PRMT5 inhibition. Multiple genome scale shRNA drop out screens performed in large tumor cell line panels have identified a strong correlation between MTAP loss and cell line dependency on PRMT5, further highlighting the strength of this metabolic vulnerability. However, PRMT5 is a known cell essential gene and conditional PRMT5 knockout and siRNA knockdown studies suggest that significant liabilities could be associated with inhibiting PRMT5 in normal tissues (e.g. pan-cytopenia, infertility, skeletal muscle loss, cardiac hypertrophy, others). Therefore, novel strategies are required to exploit this metabolic vulnerability and preferentially target PRMT5 in MTAP null tumors while sparing PRMT5 in normal tissues (MTAP WT). Targeting PRMT5 with an MTA-cooperative small molecule inhibitor could preferentially target the MTA bound state of PRMT5, enriched in MTAP null tumor cells, while providing an improved therapeutic index over normal cells where MTAP is intact and MTA levels are low.

### SUMMARY OF THE INVENTION

In one aspect, the invention provides a compound of Formula I

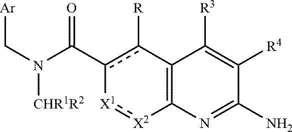

a tautomer thereof, a stereoisomer thereof, or a pharmaceutically acceptable salt of any of the foregoing,

wherein

$$\overline{- - - - - -}$$

represents a single or double bond;

X¹ and X² are both independently N or C; wherein if X¹ is C it can be optionally substituted with halo or C₁₋₆alkyl;

Ar is a six membered aromatic ring having 0-2 N atoms, wherein each Ar could be independently substituted with 0-2 R^{a} groups;
wherein R^{a} is in each instance independently selected from cyano, halo, optionally substituted C₁₋₆alkyl, C₁₋₆haloalkyl, OR^{b}, NR^{c}R^{d}, —C(O)NR^{c}R^{d}, ═S, —SO₂, —SO₂C₁₋₆alkyl, —C(O)H, —C(O)C₁₋₆alkyl, C(O)OC₁₋₆ alkyl, difluoro-pyrrolidinyl, and 4 to 6-membered heterocyclic ring, with 0-2 heteroatoms independently selected from O and N, and which heterocyclic ring can be further independently substituted with 0-2 halogen, C₁₋₆ alkyl, —C(O)H, —C(O)C₁₋₆ alkyl or optionally substituted cycloalcoxyl;
wherein each R^{b} is in each instance independently selected from H, optionally substituted C₁₋₆ alkyl, wherein the substituents can be selected from halo; or oxetanyl;
wherein each R^{c} and R^{d} is independently selected from H, C₁₋₃alkyl, C₁₋₃ haloalkyl or —CO;
wherein R^{e} in each instance is selected from H or C₁₋₆alkyl;
wherein R^{f} and R^{g} in each instance is independently selected from H and C₁₋₆alkyl;
wherein R is H or methyl;
wherein R¹ and R² are in each instance is independently selected from H, optionally substituted C₁₋₆ alkyl, optionally substituted C₁₋₆ alkynyl, —C(OR^{e}), optionally substituted single and double cyclyl having 0-3 N, S or O atoms; wherein the substituents are selected from halo, optionally substituted C₁₋₆alkyl, —C(O)NR^{f}R^{g}, OH and an optionally substituted 5-membered ring having 0-3 N atoms; or R¹ and R² and the carbon atom to which they are attached can form an optionally substituted single or double carbocyclic or heterocyclic ring, which may be saturated, partially saturated or aromatic and further wherein the heterocyclic ring includes 1, 2 or 3 heteroatoms independently selected from N, O, and S;
wherein the substituents are selected from the group of optionally substituted C₁₋₆ alkyl, halo, CN, OR^{e} and —C(OR^{e}),
provided that R¹ and R² are not both H at the same time;
and wherein R³ and R⁴ are in each instance independently selected from H, halogen, alkynyl, cyano and C₁₋₆ alkyl, optionally substituted with halo or deuterium.

In one aspect, the invention provides compounds wherein X¹ is C. In another aspect, X² can be C.

In one aspect of the invention, the compounds of the invention are represented by Formula IA:

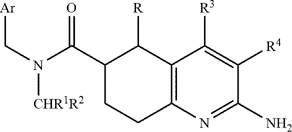

In another aspect, the compounds can be represented by Formula IB:

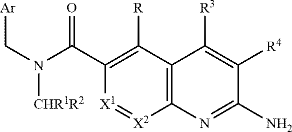

In one aspect of the invention, X² can be N. In another aspect of the invention, X¹ can be N. In a further aspect, the invention provides compounds, the tautomer thereof, the stereoisomer thereof, or the pharmaceutically acceptable salt of any of the foregoing, wherein Ar can be pyridinyl. In another aspect, Ar can be pyradazinyl. In a further aspect, Ar can be pyrazinyl. In another aspect, Ar can be phenyl.

The invention further provides compounds the tautomer thereof, the stereoisomer thereof, or the pharmaceutically acceptable salt of any of the foregoing, wherein R¹ and R² can be each independently selected from methyl and pyrimidinyl. In another aspect, R¹ and R² can be each independently selected from methyl and pyridinyl. In a further aspect, R¹ and R² can be each independently selected from H, methyl, C₁₋₆alkyl and an optionally substituted single or double cyclyl having 0-3 N, S or O items. In one aspect, the cyclyl is optionally substituted thiophenyl, optionally substituted thiazolyl or optionally substituted oxazolyl. In one aspect of the invention, R¹ and R² and the carbon atom to which they are attached can form an optionally substituted single or double carbocyclic or heterocyclic ring, which may be saturated, partially saturated or aromatic and further wherein the heterocyclic ring includes 1, 2 or 3 heteroatoms independently selected from N, O, and S.

In one aspect, R⁴ can be methyl or halogen.

In one aspect, R^{a} can be cyano, halo or optionally substituted C₁₋₆alkyl.

The invention further provides compounds, the tautomer thereof, the stereoisomer thereof, or the pharmaceutically acceptable salt of any of the foregoing, wherein the compounds are selected from:
2-amino-3-methyl-N-(2-methylpropyl)-N-((5-(trifluoromethyl)-2-pyridinyl)methyl)-6-quinolinecarboxamide,
2-amino-3-methyl-N-((2R)-3,3,3-trifluoro-2-methylpropyl)-N-((5-(trifluoromethyl)-2-pyridinyl)methyl)-6-quinolinecarboxamide,
2-amino-3-methyl-N-((2S)-3,3,3-trifluoro-2-methylpropyl)-N-((5-(trifluoromethyl)-2-pyridinyl)methyl)-6-quinolinecarboxamide,
2-amino-3-methyl-N-((2S)-3,3,3-trifluoro-2-methoxypropyl)-N-((5-(trifluoromethyl)-2-pyridinyl)methyl)-6-quinolinecarboxamide,
2-amino-3-methyl-N-((7R)-4,5,6,7-tetrahydro-1H-indazol-7-yl)-N-((5-(trifluoromethyl)-2-pyridinyl)methyl)-6-quinolinecarboxamide,
2-amino-N-((1R)-1-(6-cyano-2-pyridinyl)ethyl)-3-methyl-N-((5-(trifluoromethyl)-2-pyridinyl)methyl)-6-quinolinecarboxamide,
2-amino-N-((6-methoxy-3-pyridazinyl)methyl)-3-methyl-N-((1R)-1-(1-methyl-1H-1,2,4-triazol-3-yl)ethyl)-6-quinolinecarboxamide,
2-amino-N-((3R,4R)-4-methoxytetrahydro-2H-pyran-3-yl)-3-methyl-N-((5-(trifluoromethyl)-2-pyridinyl)methyl)-6-quinolinecarboxamide,
2-amino-N-((3S,4R)-3-fluorotetrahydro-2H-pyran-4-yl)-3-methyl-N-((5-(trifluoromethyl)-2-pyridinyl)methyl)-6-quinolinecarboxamide,
7-amino-6-bromo-N-((1R)-1-(1,3-thiazol-4-yl)ethyl)-N-((6-(trifluoromethyl)-3-pyridazinyl)methyl)-1,8-naphthyridine-3-carboxamide,
2-amino-N-((3S,4R)-3-hydroxytetrahydro-2H-pyran-4-yl)-3-methyl-N-((5-(trifluoromethyl)-2-pyridinyl)methyl)-6-quinolinecarboxamide,
2-amino-N-((3S,4R)-3-methoxytetrahydro-2H-pyran-4-yl)-3-methyl-N-((5-(trifluoromethyl)-2-pyridinyl)methyl)-6-quinolinecarboxamide,
2-amino-3-methyl-N-((1R)-1-(1,2,4-oxadiazol-3-yl)ethyl)-N-((5-(trifluoromethyl)-2-pyridinyl)methyl)-6-quinolinecarboxamide,
2-amino-N-((1R,2R)-2-cyanocyclopentyl)-3-methyl-N-((5-(trifluoromethyl)-2-pyridinyl)methyl)-6-quinolinecarboxamide,
2-amino-3-methyl-N-(4-pyrimidinylmethyl)-N-((5-(trifluoromethyl)-2-pyridinyl)methyl)-6-quinolinecarboxamide,
2-amino-3-methyl-N-(2-pyrazinylmethyl)-N-((5-(trifluoromethyl)-2-pyridinyl)methyl)-6-quinolinecarboxamide,
2-amino-3-methyl-N-((1-methyl-1H-1,2,4-triazol-3-yl)methyl)-N-((5-(trifluoromethyl)-2-pyridinyl)methyl)-6-quinolinecarboxamide,
2-amino-7-fluoro-3-methyl-N-(2-methylpropyl)-N-((5-(trifluoromethyl)-2-pyridinyl)methyl)-6-quinolinecarboxamide,
2-amino-3-methyl-N-((5-methyl-2-pyrazinyl)methyl)-N-((5-(trifluoromethyl)-2-pyridinyl)methyl)-6-quinolinecarboxamide,
2-amino-7-fluoro-3-methyl-N-(2-pyrazinylmethyl)-N-((5-(trifluoromethyl)-2-pyridinyl)methyl)-6-quinolinecarboxamide,
2-amino-7-fluoro-3-methyl-N-((5-methyl-1,2-oxazol-3-yl)methyl)-N-((5-(trifluoromethyl)-2-pyridinyl)methyl)-6-quinolinecarboxamide,
2-amino-3-methyl-N-((1R)-1-(2-pyrazinyl)ethyl)-N-((5-(trifluoromethyl)-2-pyridinyl)methyl)-6-quinolinecarboxamide,
2-amino-3-methyl-N-(2-pyrimidinylmethyl)-N-((5-(trifluoromethyl)-2-pyridinyl)methyl)-6-quinolinecarboxamide,
2-amino-N-((3-fluoro-5-(trifluoromethyl)-2-pyridinyl)methyl)-3-methyl-N-(2-methylpropyl)-6-quinolinecarboxamide,
2-amino-N-((5-chloro-2-pyrimidinyl)methyl)-3-methyl-N-((5-(trifluoromethyl)-2-pyridinyl)methyl)-6-quinolinecarboxamide,
2-amino-3-bromo-N-(2-pyrimidinylmethyl)-N-((5-(trifluoromethyl)-2-pyridinyl)methyl)-6-quinolinecarboxamide,
2-amino-3-bromo-N-(2-methylpropyl)-N-((5-(trifluoromethyl)-2-pyridinyl)methyl)-6-quinolinecarboxamide,
2-amino-7-fluoro-3-methyl-N-(2-pyrimidinylmethyl)-N-((5-(trifluoromethyl)-2-pyridinyl)methyl)-6-quinolinecarboxamide,
2-amino-N-((5-chloro-2-pyridinyl)methyl)-3-methyl-N-(2-pyrimidinylmethyl)-6-quinolinecarboxamide,
2-amino-N-((5-fluoro-2-pyrimidinyl)methyl)-3-methyl-N-((5-(trifluoromethyl)-2-pyridinyl)methyl)-6-quinolinecarboxamide,
2-amino-N-((3-fluoro-2-pyridinyl)methyl)-3-methyl-N-((5-(trifluoromethyl)-2-pyridinyl)methyl)-6-quinolinecarboxamide,
7-amino-6-bromo-N-((3-fluoro-2-pyridinyl)methyl)-N-((5-(trifluoromethyl)-2-pyridinyl)methyl)-1,8-naphthyridine-3-carboxamide,
7-amino-N-((3-fluoro-2-pyridinyl)methyl)-6-methyl-N-((5-(trifluoromethyl)-2-pyridinyl)methyl)-1,8-naphthyridine-3-carboxamide,
2-amino-N-((5-(difluoromethyl)-2-pyridinyl)methyl)-3-methyl-N-(2-methylpropyl)-6-quinolinecarboxamide,
2-amino-3-methyl-N-((1R)-1-(2-pyrimidinyl)ethyl)-N-((5-(trifluoromethyl)-2-pyridinyl)methyl)-6-quinolinecarboxamide,
2-amino-N-((3-fluoro-2-pyridinyl)methyl)-3-methyl-N-((6-(trifluoromethyl)-3-pyridinyl)methyl)-6-quinolinecarboxamide,
2-amino-N-((4R)-3,4-dihydro-2H-pyrano[3,2-b]pyridin-4-yl)-3-methyl-N-((5-(trifluoromethyl)-2-pyridinyl)methyl)-6-quinolinecarboxamide,
7-amino-6-methyl-N-((1R)-1-(2-pyrazinyl)ethyl)-N-((5-(trifluoromethyl)-2-pyridinyl)methyl)-1,8-naphthyridine-3-carboxamide,
2-amino-N-(cyclopropylmethyl)-3-methyl-N-((5-(trifluoromethyl)-2-pyridinyl)methyl)-6-quinolinecarboxamide,
2-amino-N-((5-bromo-2-pyridinyl)methyl)-3-methyl-N-(2-methylpropyl)-6-quinolinecarboxamide,
2-amino-N-((3,5-difluoro-2-pyridinyl)methyl)-3-methyl-N-((5-(trifluoromethyl)-2-pyridinyl)methyl)-6-quinolinecarboxamide,
2-amino-N-((1R)-1-(3-fluoro-2-pyridinyl)ethyl)-3-methyl-N-((5-(trifluoromethyl)-2-pyridinyl)methyl)-6-quinolinecarboxamide,
2-amino-N-((5-(difluoromethyl)-2-pyridinyl)methyl)-N-((3-fluoro-2-pyridinyl)methyl)-3-methyl-6-quinolinecarboxamide,
2-amino-N-((1S)-1-(3-fluoro-2-pyridinyl)ethyl)-3-methyl-N-((5-(trifluoromethyl)-2-pyridinyl)methyl)-6-quinolinecarboxamide,
2-amino-N-((3,5-difluoro-2-pyridinyl)methyl)-N-((3-fluoro-2-pyridinyl)methyl)-3-methyl-6-quinolinecarboxamide,
2-amino-N-((5-cyclopropyl-2-pyridinyl)methyl)-3-methyl-N-(2-methylpropyl)-6-quinolinecarboxamide, 2-amino-N-((1R)-1-(5-fluoro-2-pyridinyl)ethyl)-3-methyl-N-((5-(trifluoromethyl)-2-pyrazinyl)methyl)-6-quinolinecarboxamide,
2-amino-3-methyl-N-((1R)-1-(2-pyrimidinyl)ethyl)-N-((5-(trifluoromethyl)-2-pyrazinyl)methyl)-6-quinolinecarboxamide,
7-amino-6-bromo-N-((1R)-1-(3-fluoro-2-pyridinyl)ethyl)-N-((5-(trifluoromethyl)-2-pyridinyl)methyl)-1,8-naphthyridine-3 -carboxamide,
7-amino-N-((1R)-1-(3-fluoro-2-pyridinyl)ethyl)-6-methyl-N-((5-(trifluoromethyl)-2-pyridinyl)methyl)-1,8-naphthyridine-3 -carboxamide,
2-amino-3-methyl-N-((8R)-5,6,7,8-tetrahydro-8-quinolinyl)-N-((5-(trifluoromethyl)-2-pyridinyl)methyl)-6-quinolinecarboxamide,
2-amino-3-methyl-N-((8R)-5,6,7,8-tetrahydro-8-isoquinolinyl)-N-((5-(trifluoromethyl)-2-pyridinyl)methyl)-6-quinolinecarboxamide,
2-amino-N-((1R)-1-(3-fluoro-2-pyridinyl)ethyl)-N-((3-fluoro-5-(trifluoromethyl)-2-pyridinyl)methyl)-3-methyl-6-quinolinecarboxamide,
2-amino-N-((1R)-1-(5-fluoro-2-pyridinyl)ethyl)-3-methyl-N-((5-(trifluoromethyl)-2-pyridinyl)methyl)-6-quinolinecarboxamide,
2-amino-N-((1S)-1-(5-fluoro-2-pyridinyl)ethyl)-3-methyl-N-((5-(trifluoromethyl)-2-pyridinyl)methyl)-6-quinolinecarboxamide,
2-amino-3-methyl-N-((1R)-1-(5-methyl-1,2-oxazol-3-yl)ethyl)-N-((5-(trifluoromethyl)-2-pyridinyl)methyl)-6-quinolinecarboxamide,
2-amino-N-((3-fluoro-2-pyridinyl)methyl)-3-methyl-N-((1R)-1-(5-(trifluoromethyl)-2-pyridinyl)ethyl)-6-quinolinecarboxamide,
2-amino-N-((1R)-1-cyclopropylethyl)-3-methyl-N-((5-(trifluoromethyl)-2-pyridinyl)methyl)-6-quinolinecarboxamide,
2-amino-3-methyl-N-(2-methylpropyl)-N-((1R)-1-(5-(trifluoromethyl)-2-pyridinyl)ethyl)-6-quinolinecarboxamide,
2-amino-N-((5-cyano-2-pyridinyl)methyl)-N-((3-fluoro-2-pyridinyl)methyl)-3-methyl-6-quinolinecarboxamide,
2-amino-N-((4R)-3,4-dihydro-2H-pyrano[2,3-b]pyridin-4-yl)-3-methyl-N-((5-(trifluoromethyl)-2-pyridinyl)methyl)-6-quinolinecarboxamide,
2-amino-N-((5-bromo-2-pyridinyl)methyl)-N-((1R)-1-(3-fluoro-2-pyridinyl)ethyl)-3-methyl-6-quinolinecarboxamide,
2-amino-N-((1R)-1-(3-fluoro-2-pyridinyl)ethyl)-3-methyl-N-((5-(methylsulfonyl)-2-pyridinyl)methyl)-6-quinolinecarboxamide,
7-amino-6-methyl-N-((1R)-1-(2-pyrimidinyl)ethyl)-N-((5-(trifluoromethyl)-2-pyridinyl)methyl)-1,8-naphthyridine-3-carboxamide,
2-amino-3-methyl-N-((1R)-1-(2-pyrimidinyl)ethyl)-N-((5-(trifluoromethoxy)-2-pyridinyl)methyl)-6-quinolinecarboxamide,
2-amino-3-methyl-N-((1R)-1-(2-pyrimidinyl)ethyl)-N-((6-(trifluoromethyl)-3-pyridazinyl)methyl)-6-quinolinecarboxamide,
2-amino-N-((5-cyano-2-pyridinyl)methyl)-N-((1R)-1-(3-fluoro-2-pyridinyl)ethyl)-3-methyl-6-quinolinecarboxamide,
2-amino-N-((3-fluoro-2-pyridinyl)methyl)-3-methyl-N-(4-(trifluoromethyl)benzyl)-6-quinolinecarboxamide,
2-amino-N-((3-fluoro-2-pyridinyl)methyl)-3-methyl-N-(2-methyl-4-(trifluoromethyl)benzyl)-6-quinolinecarboxamide,
7-amino-6-bromo-N-((1R)-1-(2-pyrimidinyl)ethyl)-N-((5-(trifluoromethyl)-2-pyridinyl)methyl)-1,8-naphthyridine-3-carboxamide,
2-amino-N-((5-(cyclopropylsulfonyl)-2-pyridinyl)methyl)-N-((1R)-1-(3-fluoro-2-pyridinyl)ethyl)-3-methyl-6-quinolinecarboxamide,
2-amino-N-((5-cyano-2-pyridinyl)methyl)-3-methyl-N-((1R)-1-(2-pyrimidinyl)ethyl)-6-quinolinecarboxamide,
2-amino-N-((5-bromo-2-pyridinyl)methyl)-N-((1R)-1-(3-chloro-2-pyridinyl)ethyl)-3-methyl-6-quinolinecarboxamide,
2-amino-N-((1R)-1-(5-chloro-2-pyridinyl)ethyl)-3-methyl-N-((5-(trifluoromethyl)-2-pyridinyl)methyl)-6-quinolinecarboxamide,
2-amino-3-methyl-N-((1R)-1-(2-pyrimidinyl)propyl)-N-((5-(trifluoromethyl)-2-pyridinyl)methyl)-6-quinolinecarboxamide,
2-amino-N-((5-chloro-2-pyridinyl)methyl)-N-((1R)-1-(3 -fluoro-2-pyridinyl)ethyl)-3 -methyl-6-quinolinecarboxamide,
2-amino-N-((5-chloro-2-pyridinyl)methyl)-3-methyl-N-(2-methylpropyl)-6-quinolinecarboxamide,
2-amino-N-((1R)-1-(4-fluorophenyl)ethyl)-3-methyl-N-((5-(trifluoromethyl)-2-pyridinyl)methyl)-6-quinolinecarboxamide,
7-amino-6-ethynyl-N-((1R)-1-(3-fluoro-2-pyridinyl)ethyl)-N-((5-(trifluoromethyl)-2-pyridinyl)methyl)-1,8-naphthyridine-3 -carboxamide,
2-amino-N-((5-bromo-2-pyridinyl)methyl)-3-methyl-N-((1R)-1-(2-pyrimidinyl)ethyl)-6-quinolinecarboxamide,
2-amino-N-((7R)-6,7-dihydro-5H-cyclopenta[b]pyridin-7-yl)-3-methyl-N-((6-(trifluoromethyl)-3-pyridazinyl)methyl)-6-quinolinecarboxamide,
2-amino-7-fluoro-N-((3-fluoro-2-pyridinyl)methyl)-3-methyl-N-((5-(trifluoromethyl)-2-pyridinyl)methyl)-6-quinolinecarboxamide,
7-amino-6-bromo-N-((1R)-1-(2-pyrimidinyl)ethyl)-N-((5-(trifluoromethyl)-2-pyrazinyl)methyl)-1,8-naphthyridine-3-carboxamide,
2-amino-3-methyl-N-(2-pyridinylmethyl)-N-((5-(trifluoromethyl)-2-pyridinyl)methyl)-6-quinolinecarboxamide,
methyl 6-((((2-amino-3-methyl-6-quinolinyl)carbonyl)((3-fluoro-2-pyridinyl)methyl)amino)methyl)-3-pyridinecarboxylate,
2-amino-N-((5-(difluoromethoxy)-2-pyridinyl)methyl)-3-methyl-N-((1R)-1-(2-pyrimidinyl)ethyl)-6-quinolinecarboxamide,
2-amino-N-((3-chloro-5-(trifluoromethyl)-2-pyridinyl)methyl)-N-((1R)-1-(3-fluoro-2-pyridinyl)ethyl)-3-methyl-6-quinolinecarboxamide,
2-amino-7-fluoro-3-methyl-N-((1R)-1-(2-pyrimidinyl)ethyl)-N-((5-(trifluoromethyl)-2-pyridinyl)methyl)-6-quinolinecarboxamide,
2-amino-N-(cyclopropylmethyl)-7-fluoro-3-methyl-N-((5-(trifluoromethyl)-2-pyridinyl)methyl)-6-quinolinecarboxamide,
methyl 6-((((2-amino-3-methyl-6-quinolinyl)carbonyl)((1R)-1-(3-fluoro-2-pyridinyl)ethyl)amino)methyl)-3-pyridinecarboxylate,
2-amino-N-((5-(dimethylcarbamoyl)-2-pyridinyl)methyl)-N-((3-fluoro-2-pyridinyl)methyl)-3-methyl-6-quinolinecarboxamide,
2-amino-3-methyl-N-(1-(2-pyrimidinyl)cyclopropyl)-N-((5-(trifluoromethyl)-2-pyridinyl)methyl)-6-quinolinecarboxamide,
2-amino-3-methyl-N-((1R)-1-(2-pyrimidinyl)ethyl)-N-((5-(trifluoromethyl)-1,3-thiazol-2-yl)methyl)-6-quinolinecarboxamide,
2-amino-3-methyl-N-(3-pyridinylmethyl)-N-((5-(trifluoromethyl)-2-pyridinyl)methyl)-6-quinolinecarboxamide,
2-amino-N-((2-amino-1,3-thiazol-5-yl)methyl)-3-methyl-N-((5-(trifluoromethyl)-2-pyridinyl)methyl)-6-quinolinecarboxamide,
2-amino-N-((5-cyano-2-pyridinyl)methyl)-N-((1R)-1-(3,5-difluoro-2-pyridinyl)ethyl)-3-methyl-6-quinolinecarboxamide,
2-amino-3-methyl-N-((5-((~2~H_3_)methyloxy)-2-pyridinyl)methyl)-N-((1R)-1-(2-pyrimidinyl)ethyl)-6-quinolinecarboxamide,
2-amino-N-(4-carbamoylbenzyl)-3-methyl-N-((5-(trifluoromethyl)-2-pyridinyl)methyl)-6-quinolinecarboxamide,
2-amino-N-((5-(difluoromethyl)-2-pyridinyl)methyl)-N-((1R)-1-(5-fluoro-2-pyrimidinyl)ethyl)-3-methyl-6-quinolinecarboxamide,
2-amino-N-((5-bromo-2-pyridinyl)methyl)-3-methyl-N-((3R)-1-methyl-2-oxo-3-piperidinyl)-6-quinolinecarboxamide,
2-amino-N-((5-(dimethylcarbamoyl)-2-pyridinyl)methyl)-N-((1R)-1-(3-fluoro-2-pyridinyl)ethyl)-3-methyl-6-quinolinecarboxamide,
2-amino-N-((5-cyano-2-pyridinyl)methyl)-3-methyl-N-((1R)-1-(2-pyridinyl)ethyl)-6-quinolinecarboxamide,
2-amino-3-methyl-N-((1R)-1-(2-pyrimidinyl)ethyl)-N-((5-(trifluoromethyl)-2-pyridinyl)methyl)-1,7-naphthyridine-6-carboxamide,
2-amino-N-((3-fluoro-2-pyridinyl)methyl)-3-methyl-N-((5-(trifluoromethyl)-2-pyridinyl)methyl)-1,7-naphthyridine-6-carboxamide,
2-amino-3-methyl-N-(2-methylpropyl)-N-((5-(trifluoromethyl)-2-pyridinyl)methyl)-1,7-naphthyridine-6-carboxamide,
2-amino-N-((5-cyano-2-pyridinyl)methyl)-N-((4R)-3,4-dihydro-2H-pyrano[3,2-b]pyridin-4-yl)-3-methyl-6-quinolinecarboxamide,
2-amino-N-((5-cyano-2-pyridinyl)methyl)-3-methyl-N-((1R)-1-(2-pyrimidinyl)propyl)-6-quinolinecarboxamide,
2-amino-3-methyl-N-(4-pyridinylmethyl)-N-((5-(trifluoromethyl)-2-pyridinyl)methyl)-6-quinolinecarboxamide,
2-amino-3-methyl-N-((3-methyl-2-pyridinyl)methyl)-N-((5-(trifluoromethyl)-2-pyridinyl)methyl)-6-quinolinecarboxamide,
2-amino-3-methyl-N-((4-methyl-1,3-thiazol-2-yl)methyl)-N-((5-(trifluoromethyl)-2-pyridinyl)methyl)-6-quinolinecarboxamide,
2-amino-3-methyl-N-(4-(1-piperidinyl)benzyl)-N-(1H-pyrrolo[2,3-b]pyridin-4-ylmethyl)-6-quinolinecarboxamide,
2-amino-N-(cyclobutylmethyl)-3-methyl-N-((5-(trifluoromethyl)-2-pyridinyl)methyl)-6-quinolinecarboxamide,
2-amino-3-methyl-N-(1,2,3-thiadiazol-4-ylmethyl)-N-((5-(trifluoromethyl)-2-pyridinyl)methyl)-6-quinolinecarboxamide,
2-amino-N-(1H-indol-3-ylmethyl)-3-methyl-N-((5-(trifluoromethyl)-2-pyridinyl)methyl)-6-quinolinecarboxamide,
2-amino-3-methyl-N-((5-methyl-1,3-oxazol-4-yl)methyl)-N-((5-(trifluoromethyl)-2-pyridinyl)methyl)-6-quinolinecarboxamide,
2-amino-4-methyl-N-((1-methyl-1H-pyrazol-3-yl)methyl)-N-((5-(trifluoromethyl)-2-pyridinyl)methyl)-6-quinolinecarboxamide,
2-amino-4-methyl-N-((1-methyl-1H-1,2,4-triazol-3-yl)methyl)-N-((5-(trifluoromethyl)-2-pyridinyl)methyl)-6-quinolinecarboxamide,
2-amino-N-(2-fluorobenzyl)-3-methyl-N-(1H-pyrrolo[3,2-b]pyridin-3-ylmethyl)-6-quinolinecarboxamide,
2-amino-3-methyl-N-((4-methyl-1,3-thiazol-5-yl)methyl)-N-((5-(trifluoromethyl)-2-pyridinyl)methyl)-6-quinolinecarboxamide,
2-amino-N-(3-fluoro-4-(hydroxymethyl)benzyl)-3-methyl-N-((5-(trifluoromethyl)-2-pyridinyl)methyl)-6-quinolinecarboxamide,
2-amino-N-((6-amino-3-pyridinyl)methyl)-3-methyl-N-((5-(trifluoromethyl)-2-pyridinyl)methyl)-6-quinolinecarboxamide,
2-amino-3-methyl-N-(1H-pyrrolo[2,3-b]pyridin-4-ylmethyl)-N-((5-(trifluoromethyl)-2-pyridinyl)methyl)-6-quinolinecarboxamide,
2-amino-N-(2-fluorobenzyl)-3-methyl-N-((3-oxo-3,4-dihydro-2H-1,4-benzoxazin-6-yl)methyl)-6-quinolinecarboxamide,
2-amino-3-methyl-N-((2-oxo-2,3-dihydro-1H-benzimidazol-5-yl)methyl)-N-((5-(trifluoromethyl)-2-pyridinyl)methyl)-6-quinolinecarboxamide,
2-amino-3-methyl-N-(1,3-oxazol-4-ylmethyl)-N-((5-(trifluoromethyl)-2-pyridinyl)methyl)-6-quinolinecarboxamide,
2-amino-3-methyl-N-((3R)-1-methyl-2-oxo-3-piperidinyl)-N-((5-(trifluoromethyl)-2-pyridinyl)methyl)-6-quinolinecarboxamide,
2-amino-4-methyl-N-((1R)-1-(2-pyrimidinyl)ethyl)-N-((5-(trifluoromethyl)-2-pyridinyl)methyl)-6-quinolinecarboxamide,
2-amino-N-((5-cyano-2-pyridinyl)methyl)-7-fluoro-3-methyl-N-((1R)-1-(2-pyrimidinyl)ethyl)-6-quinolinecarboxamide,
2-amino-N-((5-(difluoromethyl)-2-pyridinyl)methyl)-3-methyl-N-((1R)-1-(2-pyrimidinyl)ethyl)-6-quinolinecarboxamide,
2-amino-3-methyl-N-((3R)-2-oxo-3-piperidinyl)-N-((5-(trifluoromethyl)-2-pyridinyl)methyl)-6-quinolinecarboxamide,
2-amino-N-((5-chloro-2-pyridinyl)methyl)-3-methyl-N-((1R)-1-(2-pyrimidinyl)ethyl)-6-quinolinecarboxamide,
2-amino-3-methyl-N-((1R)-1-(2-pyrimidinyl)propyl)-N-((6-(trifluoromethyl)-3-pyridazinyl)methyl)-6-quinolinecarboxamide,
2-amino-4-chloro-N-((1R)-1-(2-pyrimidinyl)ethyl)-N-((5-(trifluoromethyl)-2-pyridinyl)methyl)-6-quinolinecarboxamide,
2-amino-N-((3R)-1-cyclopropyl-2-oxo-3-piperidinyl)-3-methyl-N-((5-(trifluoromethyl)-2-pyridinyl)methyl)-6-quinolinecarboxamide,
2-amino-3-methyl-N-((2-methyl-6-(trifluoromethyl)-3-pyridinyl)methyl)-N-((1R)-1-(2-pyrimidinyl)ethyl)-6-quinolinecarboxamide,
2-amino-N-((6-bromo-3-pyridazinyl)methyl)-3-methyl-N-((1R)-1-(2-pyrimidinyl)ethyl)-6-quinolinecarboxamide,
2-amino-N-((5-bromo-3-methyl-2-pyridinyl)methyl)-3-methyl-N-((1R)-1-(2-pyrimidinyl)ethyl)-6-quinolinecarboxamide,
2-amino-N-((3-chloro-2-pyridinyl)methyl)-3-methyl-N-((5-(trifluoromethyl)-2-pyridinyl)methyl)-6-quinolinecarboxamide,
2-amino-N-((5-cyano-3-methyl-2-pyridinyl)methyl)-3-methyl-N-((1R)-1-(2-pyrimidinyl)ethyl)-6-quinolinecarboxamide,
2-amino-N-(4-cyanobenzyl)-3-methyl-N-((1R)-1-(2-pyrimidinyl)ethyl)-6-quinolinecarboxamide,
2-amino-N-((5-cyano-2-pyridinyl)methyl)-N-((1R)-1-(2-fluorophenyl)ethyl)-3-methyl-6-quinolinecarboxamide,
2-amino-N-((5-chloro-3-fluoro-2-pyridinyl)methyl)-3-methyl-N-((5-(trifluoromethyl)-2-pyridinyl)methyl)-6-quinolinecarboxamide,
2-amino-N-(2,6-difluorobenzyl)-3-methyl-N-((5-(trifluoromethyl)-2-pyridinyl)methyl)-6-quinolinecarboxamide,
2-amino-3-methyl-N-((1R)-1-(2-pyrimidinyl)propyl)-N-((5-(trifluoromethyl)-2-pyrazinyl)methyl)-6-quinolinecarboxamide,
2-amino-3-chloro-N-((1R)-1-(2-pyrimidinyl)ethyl)-N-((5-(trifluoromethyl)-2-pyridinyl)methyl)-6-quinolinecarboxamide,
2-amino-N-cyclopropyl-7-fluoro-3-methyl-N-((5-(trifluoromethyl)-2-pyridinyl)methyl)-6-quinolinecarboxamide,
7-amino-6-bromo-N-((5-cyano-2-pyridinyl)methyl)-N-((1R)-1-(2-pyrimidinyl)ethyl)-1,8-naphthyridine-3-carboxamide,
7-amino-6-bromo-N-((5-(difluoromethyl)-2-pyridinyl)methyl)-N-((1R)-1-(2-pyrimidinyl)ethyl)-1,8-naphthyridine-3-carboxamide,
7-amino-6-bromo-N-((1R)-1-(2-pyrimidinyl)ethyl)-N-((6-(trifluoromethyl)-3-pyridazinyl)methyl)-1,8-naphthyridine-3-carboxamide,
2-amino-7-chloro-3-methyl-N-(2-methylpropyl)-N-((5-(trifluoromethyl)-2-pyridinyl)methyl)-6-quinolinecarboxamide,
2-amino-N-((5-methoxy-2-pyrazinyl)methyl)-3-methyl-N-((1R)-1-(2-pyrimidinyl)ethyl)-6-quinolinecarboxamide,
2-amino-N-((3-fluoro-2-pyridinyl)methyl)-3-methyl-N-((5R)-2-(trifluoromethyl)-6,7-dihydro-5H-cyclopenta[b]pyridin-5-yl)-6-quinolinecarboxamide,
2-amino-N-((5-cyano-2-pyrazinyl)methyl)-3-methyl-N-((1R)-1-(2-pyrimidinyl)ethyl)-6-quinolinecarboxamide,
2-amino-N-((R)-cyclopropyl(2-pyrimidinyl)methyl)-3-methyl-N-((5-(trifluoromethyl)-2-pyridinyl)methyl)-6-quinolinecarboxamide,
2-amino-3-chloro-N-((5-cyano-2-pyridinyl)methyl)-N-((1R)-1-(2-pyrimidinyl)ethyl)-6-quinolinecarboxamide,
2-amino-N-((6-methoxy-3-pyridazinyl)methyl)-3-methyl-N-((1R)-1-(2-pyrimidinyl)ethyl)-6-quinolinecarboxamide,
7-amino-N-((5-(difluoromethyl)-2-pyridinyl)methyl)-6-methyl-N-((1R)-1-(2-pyrimidinyl)ethyl)-1,8-naphthyridine-3-carboxamide,
2-methyl-2-propanyl 6-((((2-amino-3-methyl-6-quinolinyl)carbonyl)((1R)-1-(2-pyrimidinyl)ethyl)amino)methyl)-3′,6′-dihydro[3,4′-bipyridine]-1′(2′H)-carboxylate,
2-amino-3-methyl-N-((1R)-1-(2-pyrimidinyl)ethyl)-N-((6-(trifluoromethyl)-3-pyridinyl)methyl)-6-quinolinecarboxamide,
2-amino-3,7-dimethyl-N-(2-methylpropyl)-N-((5-(trifluoromethyl)-2-pyridinyl)methyl)-6-quinolinecarboxamide,
2-amino-N-((1R)-1-(3-fluoro-2-pyridinyl)ethyl)-3-methyl-N-((6-(trifluoromethyl)-3-pyridazinyl)methyl)-6-quinolinecarboxamide,
7-amino-6-bromo-N-((1R)-1-(3-fluoro-2-pyridinyl)ethyl)-N-((6-(trifluoromethyl)-3-pyridazinyl)methyl)-1,8-naphthyridine-3-carboxamide,
N-((1′-acetyl-1′,2′,3′,6′-tetrahydro[3,4′-bipyridin]-6-yl)methyl)-2-amino-3-methyl-N-((1R)-1-(2-pyrimidinyl)ethyl)-6-quinolinecarboxamide,
2-amino-N-((5-carbamoyl-2-pyridinyl)methyl)-N-((1R)-1-(2-fluorophenyl)ethyl)-3-methyl-6-quinolinecarboxamide,
7-amino-6-methyl-N-(1H-pyrrolo[2,3-b]pyridin-4-ylmethyl)-N-((5-(trifluoromethyl)-2-pyridinyl)methyl)-1,8-naphthyridine-3 -carboxamide,
2-amino-3-methyl-N-((1R)-1-(1-methyl-1H-1,2,4-triazol-3-yl)ethyl)-N-((5-(trifluoromethyl)-2-pyridinyl)methyl)-6-quinolinecarboxamide,
2-amino-N-(1H-indazol-5-ylmethyl)-3-methyl-N-((5-(trifluoromethyl)-2-pyridinyl)methyl)-6-quinolinecarboxamide,
2-amino-3-methyl-N-((8R)-5,6,7,8-tetrahydro[1,2,4]triazolo[1,5-a]pyridin-8-yl)-N-((5-(trifluoromethyl)-2-pyridinyl)methyl)-6-quinolinecarboxamide,
7-amino-6-bromo-N-((1R)-1-(2,4-difluorophenyl)ethyl)-N-((5-(trifluoromethyl)-2-pyridinyl)methyl)-1,8-naphthyridine-3-carboxamide,
2-amino-3-methyl-N-((5-(2-oxa-6-azaspiro[3.3]heptan-6-yl)-2-pyridinyl)methyl)-N-((1R)-1-(2-pyrimidinyl)ethyl)-6-quinolinecarboxamide,
2-amino-3-(~2~H_3_)methyl-N-(2-methylpropyl)-N-((5-(trifluoromethyl)-2-pyridinyl)methyl)-6-quinolinecarboxamide,
2-amino-3-chloro-N-((1R)-1-(2-pyrimidinyl)ethyl)-N-((6-(trifluoromethyl)-3-pyridazinyl)methyl)-6-quinolinecarboxamide,
2-amino-3-methyl-N-((1R)-1-(2-pyrimidinyl)ethyl)-N-(2-quinolinylmethyl)-6-quinolinecarboxamide,
2-amino-3-methyl-N-((1R)-1-(2-pyrimidinyl)ethyl)-N-(3-quinolinylmethyl)-6-quinolinecarboxamide,
2-amino-3-bromo-N-((1R)-1-(2-pyrimidinyl)ethyl)-N-((5-(trifluoromethyl)-2-pyridinyl)methyl)-6-quinolinecarboxamide,
2-amino-N-((5-cyano-2-pyridinyl)methyl)-N-(cyclopropylmethyl)-7-fluoro-3-methyl-6-quinolinecarboxamide,
2-amino-N-((5-(3,6-dihydro-2H-pyran-4-yl)-2-pyridinyl)methyl)-3-methyl-N-((1R)-1-(2-pyrimidinyl)ethyl)-6-quinolinecarboxamide,
2-amino-3-(~2~H_3_)methyl-N-((1R)-1-(2-pyrimidinyl)ethyl)-N-((5-(trifluoromethyl)-2-pyridinyl)methyl)-6-quinolinecarboxamide,
2-amino-N-((1R,2R)-2-hydroxycyclohexyl)-3-methyl-N-((5-(trifluoromethyl)-2-pyridinyl)methyl)-6-quinolinecarboxamide,
2-amino-N-((4-cyano-2-pyridinyl)methyl)-3-methyl-N-((1R)-1-(2-pyrimidinyl)ethyl)-6-quinolinecarboxamide,
2-amino-N-((5-(5,6-dihydro-2H-pyran-3-yl)-2-pyridinyl)methyl)-3-methyl-N-((1R)-1-(2-pyrimidinyl)ethyl)-6-quinolinecarboxamide,
2-amino-N-((6-bromo-3-pyridazinyl)methyl)-3-chloro-N-((1R)-1-(2-pyrimidinyl)ethyl)-6-quinolinecarboxamide,
2-amino-N-((5-fluoro-2-pyridinyl)methyl)-3-methyl-N-((5-(trifluoromethyl)-2-pyridinyl)methyl)-6-quinolinecarboxamide,
2-amino-7-fluoro-3-methyl-N-((1R)-1-(1-methyl-1H-1,2,4-triazol-3-yl)ethyl)-N-((5-(trifluoromethyl)-2-pyridinyl)methyl)-6-quinolinecarboxamide,
2-amino-7-fluoro-3-methyl-N-((1S)-1-(1-methyl-1H-1,2,4-triazol-3-yl)ethyl)-N-((5-(trifluoromethyl)-2-pyridinyl)methyl)-6-quinolinecarboxamide,
2-amino-3-methyl-N-(2,2,2-trifluoroethyl)-N-((5-(trifluoromethyl)-2-pyridinyl)methyl)-6-quinolinecarboxamide,
2-amino-N-((4R)-3,4-dihydro-2H-pyrano[3,2-b]pyridin-4-yl)-3-methyl-N-((6-(trifluoromethyl)-3-pyridazinyl)methyl)-6-quinolinecarboxamide,
2-amino-N-((5-cyano-2-pyridinyl)methyl)-3-methyl-N-((1R)-1-(2-pyrimidinyl)ethyl)-1,7-naphthyridine-6-carboxamide,
2-amino-3-bromo-N-((5-cyano-2-pyridinyl)methyl)-N-((1R)-1-(2-pyrimidinyl)ethyl)-6-quinolinecarboxamide,
2-amino-N-(2-methoxyethyl)-3-methyl-N-((5-(trifluoromethyl)-2-pyridinyl)methyl)-6-quinolinecarboxamide,
2-amino-N-((5-ethoxy-2-pyridinyl)methyl)-3-methyl-N-((1R)-1-(2-pyrimidinyl)ethyl)-6-quinolinecarboxamide,
2-amino-7-chloro-3-methyl-N-((1R)-1-(2-pyrimidinyl)ethyl)-N-((5-(trifluoromethyl)-2-pyridinyl)methyl)-6-quinolinecarboxamide,
2-amino-N-((1R)-2-methoxy-1-(2-pyridinyl)ethyl)-3-methyl-N-((5-(trifluoromethyl)-2-pyridinyl)methyl)-6-quinolinecarboxamide,
2-amino-N-((1S)-2-methoxy-1-(2-pyridinyl)ethyl)-3-methyl-N-((5-(trifluoromethyl)-2-pyridinyl)methyl)-6-quinolinecarboxamide,
2-amino-N-((5-cyano-2-pyridinyl)methyl)-3,4-dimethyl-N-((1R)-1-(2-pyrimidinyl)ethyl)-6-quinolinecarboxamide,
2-amino-N-((5-cyano-2-pyridinyl)methyl)-3,4-dimethyl-N-((1S)-1-(2-pyrimidinyl)ethyl)-6-quinolinecarboxamide,
2-amino-N-((5-chloro-3-pyridinyl)methyl)-3-methyl-N-((5-(trifluoromethyl)-2-pyridinyl)methyl)-6-quinolinecarboxamide,
2-amino-N-(1H-indazol-4-ylmethyl)-3-methyl-N-((5-(trifluoromethyl)-2-pyridinyl)methyl)-6-quinolinecarboxamide,
7-amino-6-bromo-N-((1R)-1-(2-pyrimidinyl)propyl)-N-((5-(trifluoromethyl)-2-pyridinyl)methyl)-1,8-naphthyridine-3-carboxamide,
2-amino-N-((6-bromo-3-pyridazinyl)methyl)-7-fluoro-3-methyl-N-((1R)-1-(2-pyrimidinyl)ethyl)-6-quinolinecarboxamide,
2-amino-3-methyl-N-((2-methyl-1H-pyrrolo[2,3-b]pyridin-4-yl)methyl)-N-((5-(trifluoromethyl)-2-pyridinyl)methyl)-6-quinolinecarboxamide,
2-amino-3-bromo-N-((1R)-1-(2-pyrimidinyl)propyl)-N-((5-(trifluoromethyl)-2-pyridinyl)methyl)-1,7-naphthyridine-6-carboxamide,
7-amino-6-bromo-N-((5-(difluoromethyl)-2-pyridinyl)methyl)-N-((1R)-1-(2-pyrimidinyl)propyl)-1,8-naphthyridine-3-carboxamide,
2-amino-N-((5-(cyclopropyloxy)-2-pyridinyl)methyl)-3-methyl-N-((1R)-1-(2-pyrimidinyl)ethyl)-6-quinolinecarboxamide,
7-amino-6-bromo-N-((6-bromo-3-pyridazinyl)methyl)-N-((1R)-1-(2-pyrimidinyl)ethyl)-1,8-naphthyridine-3-carboxamide,
2-amino-N-((1R)-1-(3-fluoro-2-pyridinyl)ethyl)-3-(~2~H_3_)methyl-N-((5-(trifluoromethyl)-2-pyridinyl)methyl)-6-quinolinecarboxamide,
2-amino-N-((5-(3,3-difluoro-1-pyrrolidinyl)-2-pyridinyl)methyl)-3-methyl-N-((1R)-1-(2-pyrimidinyl)ethyl)-6-quinolinecarboxamide,
2-amino-N-((2R)-1-methoxy-2-propanyl)-3-methyl-N-((5-(trifluoromethyl)-2-pyridinyl)methyl)-6-quinolinecarboxamide,
7-amino-6-bromo-N-((6-methoxy-3-pyridazinyl)methyl)-N-((1R)-1-(2-pyrimidinyl)ethyl)-1,8-naphthyridine-3-carboxamide,
2-amino-7-fluoro-N-((6-methoxy-3-pyridazinyl)methyl)-3-methyl-N-((1R)-1-(2-pyrimidinyl)ethyl)-6-quinolinecarboxamide,
2-amino-7-fluoro-N-((6-methoxy-3-pyridazinyl)methyl)-3-methyl-N-((1S)-1-(2-pyrimidinyl)ethyl)-6-quinolinecarboxamide,
2-amino-3-bromo-N-((5-(difluoromethyl)-2-pyridinyl)methyl)-N-((1R)-1-(2-pyrimidinyl)ethyl)-6-quinolinecarboxamide,
2-amino-3-bromo-N-((6-methoxy-3-pyridazinyl)methyl)-N-((1R)-1-(2-pyrimidinyl)ethyl)-6-quinolinecarboxamide,
2-amino-N-((5-cyano-2-pyridinyl)methyl)-3-(~2~H_3_)methyl-N-((1R)-1-(2-pyrimidinyl)ethyl)-6-quinolinecarboxamide,
2-amino-N-((5-bromo-6-methyl-2-pyridinyl)methyl)-3-methyl-N-((1R)-1-(2-pyrimidinyl)ethyl)-6-quinolinecarboxamide,
2-amino-3-bromo-N-((1R)-1-(2-pyrimidinyl)ethyl)-N-((5-(trifluoromethyl)-2-pyridinyl)methyl)-1,7-naphthyridine-6-carboxamide,
7-amino-N-((R)-cyclopropyl(2-pyrimidinyl)methyl)-6-methyl-N-((5-(trifluoromethyl)-2-pyridinyl)methyl)-1,8-naphthyridine-3-carboxamide,
2-amino-N-((6-(dimethylamino)-3-pyridazinyl)methyl)-3-methyl-N-((1R)-1-(2-pyrimidinyl)ethyl)-6-quinolinecarboxamide,
2-amino-N-((5-cyano-2-pyridinyl)methyl)-3-methyl-N-((1R)-1-(1-methyl-1H-1,2,4-triazol-3-yl)ethyl)-6-quinolinecarboxamide,
2-amino-3-bromo-N-((5-(difluoromethyl)-2-pyridinyl)methyl)-N-((1R)-1-(2-pyrimidinyl)propyl)-6-quinolinecarboxamide,
2-amino-3-methyl-N-((6-(4-morpholinyl)-3-pyridazinyl)methyl)-N-((1R)-1-(2-pyrimidinyl)ethyl)-6-quinolinecarboxamide,
2-amino-N-((6-ethoxy-3-pyridazinyl)methyl)-3-methyl-N-((1R)-1-(2-pyrimidinyl)ethyl)-6-quinolinecarboxamide,
methyl 6-((((2-amino-3-methyl-6-quinolinyl)carbonyl)((1R)-1-(2-pyrimidinyl)ethyl)amino)methyl)-3’,6′-dihydro[3,4′-bipyridine]-1′(2′H)-carboxylate,
2-amino-N-((1R,2S)-3,3-difluoro-2-hydroxycyclohexyl)-3-methyl-N-((5-(trifluoromethyl)-2-pyridinyl)methyl)-6-quinolinecarboxamide,
2-amino-N-((5-(3,3-difluoro-1-azetidinyl)-2-pyridinyl)methyl)-3-methyl-N-((1R)-1-(2-pyrimidinyl)ethyl)-6-quinolinecarboxamide,
7-amino-6-bromo-N-((R)-cyclopropyl(2-pyrimidinyl)methyl)-N-((5-(trifluoromethyl)-2-pyridinyl)methyl)-1,8-naphthyridine-3-carboxamide,
7-amino-6-bromo-N-((2S)-3,3,3-trifluoro-2-methoxypropyl)-N-((5-(trifluoromethyl)-2-pyridinyl)methyl)-1,8-naphthyridine-3-carboxamide,
2-amino-N-((5-(difluoromethyl)-2-pyridinyl)methyl)-3-methyl-N-((1R)-1-(1-methyl-1H-1,2,4-triazol-3-yl)ethyl)-6-quinolinecarboxamide,
2-amino-N-(1,3-dimethoxy-2-propanyl)-3-methyl-N-((5-(trifluoromethyl)-2-pyridinyl)methyl)-6-quinolinecarboxamide,
2-amino-N-((1R)-1-(5-fluoro-2-pyrimidinyl)ethyl)-3-methyl-N-((5-(trifluoromethyl)-2-pyridinyl)methyl)-6-quinolinecarboxamide,
2-amino-3-methyl-N-((1R)-1-(2-pyrimidinyl)ethyl)-N-((5-(tetrahydro-2H-pyran-4-yl)-2-pyridinyl)methyl)-6-quinolinecarboxamide,
2-amino-3-methyl-N-((5R)-5,6,7,8-tetrahydro-5-quinoxalinyl)-N-((5-(trifluoromethyl)-2-pyridinyl)methyl)-6-quinolinecarboxamide,
2-amino-3-methyl-N-((5 S)-5,6,7,8-tetrahydro-5-quinoxalinyl)-N-((5-(trifluoromethyl)-2-pyridinyl)methyl)-6-quinolinecarboxamide,
2-amino-3-methyl-N-((1R)-1-(2-pyrimidinyl)ethyl)-N-((6-((2,2,2-trifluoroethyl)amino)-3-pyridazinyl)methyl)-6-quinolinecarboxamide,
2-amino-N-((5-cyano-2-pyridinyl)methyl)-3-methyl-N-((8R)-5,6,7,8-tetrahydro[1,2,4]triazolo[1,5-a]pyridin-8-yl)-6-quinolinecarboxamide,
2-amino-N-((3-fluoro-2-pyridinyl)methyl)-3-methyl-N-((3S)-6-(trifluoromethyl)-2,3-dihydro-1-benzofuran-3-yl)-6-quinolinecarboxamide,
2-amino-3-bromo-N-((5-chloro-2-pyridinyl)methyl)-N-((1R)-1-(2-pyrimidinyl)ethyl)-6-quinolinecarboxamide,
2-amino-3-chloro-N-((6-methoxy-3-pyridazinyl)methyl)-N-((1R)-1-(2-pyrimidinyl)ethyl)-6-quinolinecarboxamide,
7-amino-6-bromo-N-((5-chloro-2-pyridinyl)methyl)-N-((1R)-1-(2-pyrimidinyl)ethyl)-1,8-naphthyridine-3-carboxamide,
2-amino-N-((5-chloro-2-pyridinyl)methyl)-7-fluoro-3-methyl-N-((1R)-1-(2-pyrimidinyl)ethyl)-6-quinolinecarboxamide,
2-amino-N-((1R)-1-(3-chloro-2-pyridinyl)ethyl)-3-methyl-N-((6-(trifluoromethyl)-3-pyridazinyl)methyl)-6-quinolinecarboxamide,
7-amino-6-chloro-N-((1R)-1-(2-pyrimidinyl)ethyl)-N-((5-(trifluoromethyl)-2-pyridinyl)methyl)-1,8-naphthyridine-3-carboxamide,
2-amino-N-((1R,2R)-2-hydroxycyclopentyl)-3-methyl-N-((5-(trifluoromethyl)-2-pyridinyl)methyl)-6-quinolinecarboxamide,
2-amino-N-((1R,2R)-2-hydroxycyclopentyl)-3-methyl-N-((6-(trifluoromethyl)-3-pyridazinyl)methyl)-6-quinolinecarboxamide,
7-amino-6-chloro-N-((5-(difluoromethyl)-2-pyridinyl)methyl)-N-((1R)-1-(2-pyrimidinyl)ethyl)-1,8-naphthyridine-3-carboxamide,
2-amino-N-((R)-cyclopropyl(2-pyrimidinyl)methyl)-3-methyl-N-((6-(trifluoromethyl)-3-pyridazinyl)methyl)-6-quinolinecarboxamide,
2-amino-N-((5-cyano-2-pyridinyl)methyl)-N-((R)-cyclopropyl(2-pyrimidinyl)methyl)-7-fluoro-3-methyl-6-quinolinecarboxamide,
2-amino-3-chloro-N-((5-cyano-2-pyridinyl)methyl)-N-((1R)-1-(3-fluoro-2-pyridinyl)ethyl)-6-quinolinecarboxamide,
2-amino-3-bromo-N-((5-cyano-2-pyridinyl)methyl)-N-((1R)-1-(3-fluoro-2-pyridinyl)ethyl)-6-quinolinecarboxamide,
2-amino-N-((4R)-6-fluoro-3,4-dihydro-2H-pyrano[3,2-b]pyridin-4-yl)-3-methyl-N-((5-(trifluoromethyl)-2-pyridinyl)methyl)-6-quinolinecarboxamide,
2-amino-N-((5-cyano-2-pyridinyl)methyl)-7-fluoro-N-((1R)-1-(3-fluoro-2-pyridinyl)ethyl)-3-methyl-6-quinolinecarboxamide,
2-amino-N-((6-(3,6-dihydro-2H-pyran-4-yl)-3-pyridazinyl)methyl)-3-methyl-N-((1R)-1-(2-pyrimidinyl)ethyl)-6-quinolinecarboxamide,
2-amino-3-chloro-N-((6-(3,6-dihydro-2H-pyran-4-yl)-3-pyridazinyl)methyl)-N-((1R)-1-(2-pyrimidinyl)ethyl)-6-quinolinecarboxamide,
7-amino-6-bromo-N-((5-cyano-2-pyridinyl)methyl)-N-((1R)-1-(3-fluoro-2-pyridinyl)ethyl)-1,8-naphthyridine-3-carboxamide,
7-amino-N-((1R)-1-(5-fluoro-2-pyrimidinyl)ethyl)-6-methyl-N-((5-(trifluoromethyl)-2-pyridinyl)methyl)-1,8-naphthyridine-3 -carboxamide,
2-amino-N-((6-bromo-3-pyridazinyl)methyl)-3-methyl-N-((1R)-1-(1,3-thiazol-2-yl)ethyl)-6-quinolinecarboxamide,
2-amino-3-methyl-N-((1R)-1-(1,3-thiazol-4-yl)ethyl)-N-((6-(trifluoromethyl)-3-pyridazinyl)methyl)-6-quinolinecarboxamide,
2-amino-7-chloro-N-((5-cyano-2-pyridinyl)methyl)-3-methyl-N-((1R)-1-(2-pyrimidinyl)ethyl)-6-quinolinecarboxamide,
2-amino-N-((6-bromo-3-pyridazinyl)methyl)-3-methyl-N-((8R)-5,6,7,8-tetrahydro[1,2,4]triazolo[1,5-a]pyridin-8-yl)-6-quinolinecarboxamide,
2-amino-3-methyl-N-((1R)-1-(2-pyrimidinyl)ethyl)-N-((6-(trifluoromethyl)-2-pyridinyl)methyl)-6-quinolinecarboxamide,
2-amino-N-((6-(difluoromethyl)-3-pyridazinyl)methyl)-3-methyl-N-((1R)-1-(2-pyrimidinyl)ethyl)-6-quinolinecarboxamide,
2-amino-N-((5-(difluoromethyl)-2-pyridinyl)methyl)-N-((1R,2R)-2-hydroxycyclopentyl)-3-methyl-6-quinolinecarboxamide,
2-amino-N-((5-cyano-2-pyridinyl)methyl)-N-((1R,2R)-2-hydroxycyclopentyl)-3-methyl-6-quinolinecarboxamide,
2-amino-N-((6-bromo-3-pyridazinyl)methyl)-N-((1R,2R)-2-hydroxycyclopentyl)-3-methyl-6-quinolinecarboxamide,
2-amino-N-((1R,2R)-2-hydroxycyclopentyl)-N-((6-methoxy-3-pyridazinyl)methyl)-3-methyl-6-quinolinecarboxamide,
2-amino-3-chloro-N-((1R)-1-(2-pyrimidinyl)ethyl)-N-((5-(trifluoromethyl)-2-pyridinyl)methyl)-1,7-naphthyridine-6-carboxamide,
2-amino-N-((5-(difluoromethyl)-2-pyridinyl)methyl)-3-methyl-N-((1R)-1-(5-methyl-1,2,4-oxadiazol-3-yl)ethyl)-6-quinolinecarboxamide,
2-amino-3-bromo-N-((6-bromo-3-pyridazinyl)methyl)-N-((1R)-1-(1,3-thiazol-2-yl)ethyl)-6-quinolinecarboxamide,
2-amino-3-chloro-N-((5-(difluoromethyl)-2-pyridinyl)methyl)-N-((1R)-1-(2-pyrimidinyl)ethyl)-6-quinolinecarboxamide,
2-amino-3-iodo-N-((1R)-1-(2-pyrimidinyl)ethyl)-N-((5-(trifluoromethyl)-2-pyridinyl)methyl)-6-quinolinecarboxamide,
2-amino-N-((5-cyano-2-pyridinyl)methyl)-N-((R)-cyclopropyl(2-pyrimidinyl)methyl)-3-methyl-6-quinolinecarboxamide,
7-amino-6-bromo-N-((6-bromo-3-pyridazinyl)methyl)-N-((1R)-1-(3-fluoro-2-pyridinyl)ethyl)-1,8-naphthyridine-3-carboxamide,
7-amino-6-bromo-N-((5-cyano-2-pyridinyl)methyl)-N-((R)-cyclopropyl(2-pyrimidinyl)methyl)-1,8-naphthyridine-3-carboxamide,
7-amino-6-bromo-N-((5-(difluoromethyl)-2-pyridinyl)methyl)-N-((1R)-1-(1-methyl-1H-1,2,4-triazol-3-yl)ethyl)-1,8-naphthyridine-3-carboxamide,
2-amino-N-((6-bromo-3-pyridazinyl)methyl)-3-methyl-N-((1R)-1-(1-methyl-1H-1,2,4-triazol-3-yl)ethyl)-6-quinolinecarboxamide,
2-amino-N-((4R)-6-fluoro-3,4-dihydro-2H-pyrano [3,2-b]pyridin-4-yl)-3-methyl-N-((6-(trifluoromethyl)-3-pyridazinyl)methyl)-6-quinolinecarboxamide,
N-((5-(1-acetyl-4-piperidinyl)-2-pyridinyl)methyl)-2-amino-3-methyl-N-((1R)-1-(2-pyrimidinyl)ethyl)-6-quinolinecarboxamide,
7-amino-6-bromo-N-((5-(3-oxetanyloxy)-2-pyridinyl)methyl)-N-((1R)-1-(2-pyrimidinyl)ethyl)-1,8-naphthyridine-3-carboxamide,
2-amino-3-methyl-N-((5-(3-oxetanyloxy)-2-pyridinyl)methyl)-N-((1R)-1-(2-pyrimidinyl)ethyl)-6-quinolinecarboxamide,
7-amino-6-bromo-N-((5R)-5,6,7,8-tetrahydro-5-quinoxalinyl)-N-((5-(trifluoromethyl)-2-pyridinyl)methyl)-1,8-naphthyridine-3-carboxamide,
2-amino-N-((5-chloro-6-methyl-2-pyridinyl)methyl)-3-methyl-N-((1R)-1-(2-pyrimidinyl)ethyl)-6-quinolinecarboxamide,
2-amino-N-((2-methoxy-6-(trifluoromethyl)-3-pyridinyl)methyl)-3-methyl-N-((1R)-1-(2-pyrimidinyl)ethyl)-6-quinolinecarboxamide,
7-amino-6-bromo-N-((6-ethoxy-3-pyridazinyl)methyl)-N-((1R)-1-(2-pyrimidinyl)ethyl)-1,8-naphthyridine-3-carboxamide,
2-amino-N-((3R,4S)-4-hydroxytetrahydro-3-furanyl)-3-methyl-N-((5-(trifluoromethyl)-2-pyridinyl)methyl)-6-quinolinecarboxamide,
7-amino-6-bromo-N-((5-bromo-6-methyl-2-pyridinyl)methyl)-N-((1R)-1-(2-pyrimidinyl)ethyl)-1,8-naphthyridine-3-carboxamide,
2-amino-N-((3R,4R)-4-hydroxytetrahydro-2H-pyran-3-yl)-3-methyl-N-((5-(trifluoromethyl)-2-pyridinyl)methyl)-6-quinolinecarboxamide,
2-amino-N-((1S)-1-(6-cyano-2-pyridinyl)ethyl)-3-methyl-N-((5-(trifluoromethyl)-2-pyridinyl)methyl)-6-quinolinecarboxamide,
2-amino-N-((6-methoxy-3-pyridazinyl)methyl)-3-methyl-N-((1S)-1-(1-methyl-1H-1,2,4-triazol-3-yl)ethyl)-6-quinolinecarboxamide,
2-amino-N-((3S,4S)-4-methoxytetrahydro-2H-pyran-3-yl)-3-methyl-N-((5-(trifluoromethyl)-2-pyridinyl)methyl)-6-quinolinecarboxamide,
2-amino-N-((2R)-1-methoxy-2-propanyl)-N-((6-methoxy-3-pyridazinyl)methyl)-3-methyl-6-quinolinecarboxamide,
2-amino-3-methyl-N-((1R)-2-methyl-1-(2-pyrimidinyl)propyl)-N-((5-(trifluoromethyl)-2-pyridinyl)methyl)-6-quinolinecarboxamide,
2-amino-3-methyl-N-((1S)-2-methyl-1-(2-pyrimidinyl)propyl)-N-((5-(trifluoromethyl)-2-pyridinyl)methyl)-6-quinolinecarboxamide,
7-amino-6-iodo-N-((1R)-1-(2-pyrimidinyl)ethyl)-N-((5-(trifluoromethyl)-2-pyridinyl)methyl)-1,8-naphthyridine-3-carboxamide,
2-amino-3-methyl-N-((1R)-1-(2-pyrimidinyl)ethyl)-N-((6-(2,2,2-trifluoroethoxy)-3-pyridazinyl)methyl)-6-quinolinecarboxamide,
methyl 4-(6-((((2-amino-3-methyl-6-quinolinyl)carbonyl)((1R)-1-(2-pyrimidinyl)ethyl)amino)methyl)-3-pyridinyl)-1-piperidinecarboxylate,
2-amino-3-methyl-N-((8R)-5,6,7,8-tetrahydroimidazo[1,2-a]pyridin-8-yl)-N-((6-(trifluoromethyl)-3-pyridazinyl)methyl)-6-quinolinecarboxamide,
2-amino-N-((1R,2R)-2-methoxy-2,3-dihydro-1H-inden-1-yl)-3-methyl-N-((5-(trifluoromethyl)-2-pyridinyl)methyl)-6-quinolinecarboxamide,
2-amino-N-((3-chloro-2-pyridinyl)methyl)-7-fluoro-3-methyl-N-((1R)-1-(2-pyrimidinyl)ethyl)-6-quinolinecarboxamide,
2-amino-N-((6-methoxy-3-pyridazinyl)methyl)-3-methyl-N-((2S)-3,3,3-trifluoro-2-methoxypropyl)-6-quinolinecarboxamide,
2-amino-3-bromo-N-((6-bromo-3-pyridazinyl)methyl)-N-((1R)-1-(2-pyrimidinyl)ethyl)-6-quinolinecarboxamide,
2-amino-N-((1R)-1-(2-fluoro-4-pyridinyl)ethyl)-3-methyl-N-((5-(trifluoromethyl)-2-pyridinyl)methyl)-6-quinolinecarboxamide,
2-amino-N-((1S)-1-(2-fluoro-4-pyridinyl)ethyl)-3-methyl-N-((5-(trifluoromethyl)-2-pyridinyl)methyl)-6-quinolinecarboxamide,
2-amino-3-bromo-N-((5-cyano-2-pyridinyl)methyl)-N-((2R)-1-methoxy-2-propanyl)-6-quinolinecarboxamide,
2-amino-N-((1S,2S)-2-methoxy-2,3-dihydro-1H-inden-1-yl)-3-methyl-N-((5-(trifluoromethyl)-2-pyridinyl)methyl)-6-quinolinecarboxamide,
2-amino-N-((R)-cyclopropyl(2-pyrimidinyl)methyl)-N-((6-methoxy-3-pyridazinyl)methyl)-3-methyl-6-quinolinecarboxamide,
2-amino-3-methyl-N-(2-(trifluoromethoxy)ethyl)-N-((5-(trifluoromethyl)-2-pyridinyl)methyl)-6-quinolinecarboxamide,
2-amino-N-((1R)-1-(2-pyrimidinyl)ethyl)-N-((5-(trifluoromethyl)-2-pyridinyl)methyl)-6-quinolinecarboxamide,
2-amino-N-((6-cyano-3-pyridazinyl)methyl)-N-((1R)-1-(3-fluoro-2-pyridinyl)ethyl)-3-methyl-6-quinolinecarboxamide,
2-amino-N-((1R,2R)-4,4-difluoro-2-hydroxycyclopentyl)-3-methyl-N-((5-(trifluoromethyl)-2-pyridinyl)methyl)-6-quinolinecarboxamide,
2-amino-3-bromo-N-((6-ethoxy-3-pyridazinyl)methyl)-N-((1R)-1-(2-pyrimidinyl)ethyl)-6-quinolinecarboxamide,
2-amino-3,5-dimethyl-N-((1R)-1-(2-pyrimidinyl)ethyl)-N-((5-(trifluoromethyl)-2-pyridinyl)methyl)-6-quinolinecarboxamide,
2-amino-N-((2R)-1-methoxy-2-propanyl)-3,5-dimethyl-N-((5-(trifluoromethyl)-2-pyridinyl)methyl)-6-quinolinecarboxamide,
2-amino-N-((3-chloro-2-pyridinyl)methyl)-3-methyl-N-((1R)-1-(2-pyrimidinyl)ethyl)-6-quinolinecarboxamide,
2-amino-N-((3-chloro-2-pyridinyl)methyl)-3-methyl-N-((1S)-1-(2-pyrimidinyl)ethyl)-6-quinolinecarboxamide,
2-amino-N-((5S)-5,8-dihydro-6H-pyrano[3,4-b]pyridin-5-yl)-3-methyl-N-((5-(trifluoromethyl)-2-pyridinyl)methyl)-6-quinolinecarboxamide,
N-((5-acetyl-2-pyridinyl)methyl)-2-amino-3-methyl-N-((1R)-1-(2-pyrimidinyl)ethyl)-6-quinolinecarboxamide,
2-amino-N-((5-(2-hydroxy-2-propanyl)-2-pyridinyl)methyl)-3-methyl-N-((1R)-1-(2-pyrimidinyl)ethyl)-6-quinolinecarboxamide,
2-amino-N-((1R)-1-(3-fluoro-2-pyridinyl)ethyl)-3-methyl-N-((6-(4-morpholinyl)-3-pyridazinyl)methyl)-6-quinolinecarboxamide,
7-amino-6-bromo-N-((1R)-1-(3-fluoro-2-pyridinyl)ethyl)-N-((6-methoxy-3-pyridazinyl)methyl)-1,8-naphthyridine-3-carboxamide,
2-amino-3-bromo-N-((1R)-1-(3-fluoro-2-pyridinyl)ethyl)-N-((6-methoxy-3-pyridazinyl)methyl)-6-quinolinecarboxamide,
2-amino-3-bromo-N-((6-methoxy-3-pyridazinyl)methyl)-N-((1R)-1-(1-methyl-1H-1,2,4-triazol-3-yl)ethyl)-6-quinolinecarboxamide,
2-amino-7-chloro-N-((3-fluoro-2-pyridinyl)methyl)-3-methyl-N-((5-(trifluoromethyl)-2-pyridinyl)methyl)-6-quinolinecarboxamide,
2-amino-N-((3R,4S)-3-fluorotetrahydro-2H-pyran-4-yl)-3-methyl-N-((5-(trifluoromethyl)-2-pyridinyl)methyl)-6-quinolinecarboxamide,
7-amino-6-bromo-N-((1S)-1-(1,3-thiazol-4-yl)ethyl)-N-((6-(trifluoromethyl)-3-pyridazinyl)methyl)-1,8-naphthyridine-3-carboxamide,
2-amino-N-((6-methoxy-3-pyridinyl)methyl)-3-methyl-N-((1R)-1-(2-pyrimidinyl)ethyl)-6-quinolinecarboxamide,
2-amino-N-((5-cyclopropyl-2-pyridinyl)methyl)-3-methyl-N-((1R)-1-(2-pyrimidinyl)ethyl)-6-quinolinecarboxamide,
7-amino-6-bromo-N-((1R)-1-(3-fluoro-2-pyridinyl)ethyl)-N-((6-(4-morpholinyl)-3-pyridazinyl)methyl)-1,8-naphthyridine-3 -carboxamide,
2-amino-N-((3R,4S)-3-hydroxytetrahydro-2H-pyran-4-yl)-3-methyl-N-((5-(trifluoromethyl)-2-pyridinyl)methyl)-6-quinolinecarboxamide,
2-amino-N-((6-bromo-3-pyridazinyl)methyl)-3-iodo-N-((1R)-1-(2-pyrimidinyl)ethyl)-6-quinolinecarboxamide,
2-amino-3-bromo-N-((1R)-1-(3-fluoro-2-pyridinyl)ethyl)-N-((6-(trifluoromethyl)-3-pyridazinyl)methyl)-6-quinolinecarboxamide,
2-amino-N-((3R,4S)-3-methoxytetrahydro-2H-pyran-4-yl)-3-methyl-N-((5-(trifluoromethyl)-2-pyridinyl)methyl)-6-quinolinecarboxamide,
2-amino-N-(3,4-dihydro-2H-pyrano[2,3-c]pyridin-6-ylmethyl)-3-methyl-N-((1R)-1-(2-pyrimidinyl)ethyl)-6-quinolinecarboxamide,
2-amino-N-(3,4-dihydro-2H-pyrano[2,3-c]pyridin-6-ylmethyl)-3-methyl-N-((1S)-1-(2-pyrimidinyl)ethyl)-6-quinolinecarboxamide,
2-amino-3-bromo-N-((5-cyano-2-pyridinyl)methyl)-N-((R)-cyclopropyl(2-pyrimidinyl)methyl)-6-quinolinecarboxamide,
2-amino-N-((6-(difluoromethoxy)-3-pyridazinyl)methyl)-3-methyl-N-((1R)-1-(2-pyrimidinyl)ethyl)-6-quinolinecarboxamide,
2-amino-N-((6-(difluoromethoxy)-3-pyridazinyl)methyl)-3-methyl-N-((1S)-1-(2-pyrimidinyl)ethyl)-6-quinolinecarboxamide,
2-amino-N-((6-bromo-3-pyridazinyl)methyl)-7-fluoro-3-methyl-N-((8R)-5,6,7,8-tetrahydro[1,2,4]triazolo[1,5-a]pyridin-8-yl)-6-quinolinecarboxamide,
2-amino-3-methyl-N-((1S)-1-(1,2,4-oxadiazol-3-yl)ethyl)-N-((5-(trifluoromethyl)-2-pyridinyl)methyl)-6-quinolinecarboxamide,
2-amino-3-methyl-N-((1R)-1-(2-pyrimidinyl)ethyl)-N-((6-(trifluoromethoxy)-3-pyridazinyl)methyl)-6-quinolinecarboxamide,
2-amino-3-methyl-N-((1S)-1-(2-pyrimidinyl)ethyl)-N-((6-(trifluoromethoxy)-3-pyridazinyl)methyl)-6-quinolinecarboxamide,
2-amino-N-(3-isoquinolinylmethyl)-3-methyl-N-((1R)-1-(2-pyrimidinyl)ethyl)-6-quinolinecarboxamide,
2-amino-N-(3-isoquinolinylmethyl)-3-methyl-N-((1S)-1-(2-pyrimidinyl)ethyl)-6-quinolinecarboxamide,
7-amino-6-bromo-N-((1R,2R)-2-methoxycyclohexyl)-N-((5-(trifluoromethyl)-2-pyridinyl)methyl)-1,8-naphthyridine-3-carboxamide,
7-amino-6-bromo-N-((1S,2S)-2-methoxycyclohexyl)-N-((5-(trifluoromethyl)-2-pyridinyl)methyl)-1,8-naphthyridine-3-carboxamide,
2-amino-N-((6-bromo-3-pyridazinyl)methyl)-3-chloro-N-((1R)-1-(3-fluoro-2-pyridinyl)ethyl)-6-quinolinecarboxamide,
2-amino-3-bromo-N-((6-bromo-3-pyridazinyl)methyl)-N-((8R)-5,6,7,8-tetrahydro[1,2,4]triazolo[1,5-a]pyridin-8-yl)-6-quinolinecarboxamide,
7-amino-6-iodo-N-((1R)-1-(2-pyrimidinyl)propyl)-N-((5-(trifluoromethyl)-2-pyridinyl)methyl)-1,8-naphthyridine-3-carboxamide,
2-amino-3-methyl-N-(4-(3-oxetanyl)benzyl)-N-((1R)-1-(2-pyrimidinyl)ethyl)-6-quinolinecarboxamide,
2-amino-3-methyl-N-(4-(3-oxetanyl)benzyl)-N-((1S)-1-(2-pyrimidinyl)ethyl)-6-quinolinecarboxamide,
2-amino-N-((1R)-1-cyclopropyl-2-methoxyethyl)-3-methyl-N-((5-(trifluoromethyl)-2-pyridinyl)methyl)-6-quinolinecarboxamide,
2-amino-N-((1S)-1-cyclopropyl-2-methoxyethyl)-3-methyl-N-((5-(trifluoromethyl)-2-pyridinyl)methyl)-6-quinolinecarboxamide,
2-amino-N-((3R,4R)-4-fluorotetrahydro-2H-pyran-3-yl)-3-methyl-N-((5-(trifluoromethyl)-2-pyridinyl)methyl)-6-quinolinecarboxamide,
2-amino-N-((1R,2S)-2-cyanocyclopentyl)-3-methyl-N-((5-(trifluoromethyl)-2-pyridinyl)methyl)-6-quinolinecarboxamide,
2-amino-N-((1S,2R)-2-cyanocyclopentyl)-3-methyl-N-((5-(trifluoromethyl)-2-pyridinyl)methyl)-6-quinolinecarboxamide,
2-amino-N-((1S,2S)-2-cyanocyclopentyl)-3-methyl-N-((5-(trifluoromethyl)-2-pyridinyl)methyl)-6-quinolinecarboxamide,
2-amino-N-((1R)-1-(6-fluoro-2-pyridinyl)ethyl)-3-methyl-N-((5-(trifluoromethyl)-2-pyridinyl)methyl)-6-quinolinecarboxamide,
7-amino-6-bromo-N-((1R)-1-(2-pyrimidinyl)ethyl)-N-((6-(2,2,2-trifluoroethoxy)-3-pyridazinyl)methyl)-1,8-naphthyridine-3 -carboxamide,
2-amino-7-fluoro-3-methyl-N-((6-(4-morpholinyl)-3-pyridazinyl)methyl)-N-((1R)-1-(2-pyrimidinyl)ethyl)-6-quinolinecarboxamide,
7-amino-N-((5-chloro-2-pyridinyl)methyl)-6-iodo-N-((1R)-1-(2-pyrimidinyl)ethyl)-1,8-naphthyridine-3-carboxamide,
7-amino-6-bromo-N-((1R)-1-(6-fluoro-2-pyridinyl)ethyl)-N-((5-(trifluoromethyl)-2-pyridinyl)methyl)-1,8-naphthyridine-3 -carboxamide,
2-amino-3-methyl-N-((5-(4-morpholinyl)-2-pyridinyl)methyl)-N-((1R)-1-(2-pyrimidinyl)ethyl)-6-quinolinecarboxamide,
2-amino-3-bromo-N-((5-cyano-2-pyridinyl)methyl)-N-((1R)-1-(2-pyrimidinyl)propyl)-6-quinolinecarboxamide,
2-amino-N-((6-cyclopropyl-3-pyridazinyl)methyl)-3-methyl-N-((1R)-1-(2-pyrimidinyl)ethyl)-6-quinolinecarboxamide,
2-amino-N-((1R,2R)-2-(difluoromethoxy)cyclopentyl)-3-methyl-N-((5-(trifluoromethyl)-2-pyridinyl)methyl)-6-quinolinecarboxamide,
2-amino-N-((1S,2S)-2-(difluoromethoxy)cyclopentyl)-3-methyl-N-((5-(trifluoromethyl)-2-pyridinyl)methyl)-6-quinolinecarboxamide,
2-amino-N-((6-chloro-3-pyridazinyl)methyl)-3-methyl-N-((1R)-1-(2-pyrimidinyl)ethyl)-6-quinolinecarboxamide,
2-amino-N-((6-cyano-2-methyl-3-pyridinyl)methyl)-3-methyl-N-((1R)-1-(2-pyrimidinyl)ethyl)-6-quinolinecarboxamide,
2-amino-N-(2-methylpropyl)-3-phenyl-N-((5-(trifluoromethyl)-2-pyridinyl)methyl)-6-quinolinecarboxamide,
7-amino-6-bromo-5-methyl-N-((1R)-1-(2-pyrimidinyl)ethyl)-N-((5-(trifluoromethyl)-2-pyridinyl)methyl)-1,8-naphthyridine-3-carboxamide,
2-amino-N-((5-(difluoromethyl)-2-pyridinyl)methyl)-3-iodo-N-((1R)-1-(1-methyl-1H-1,2,4-triazol-3-yl)ethyl)-6-quinolinecarboxamide,
2-amino-3-bromo-N-((6-cyclopropyl-3-pyridazinyl)methyl)-N-((1R)-1-(2-pyrimidinyl)ethyl)-6-quinolinecarboxamide,
2-amino-N-((4-chloro-5-cyano-2-pyridinyl)methyl)-3-methyl-N-((1R)-1-(2-pyrimidinyl)ethyl)-6-quinolinecarboxamide,
2-amino-N-((1R)-1-(3-fluoro-2-pyridinyl)ethyl)-3-methyl-N-((6-(methylamino)-3-pyridazinyl)methyl)-6-quinolinecarboxamide,
7-amino-6-bromo-N-((5-cyano-2-pyridinyl)methyl)-5-methyl-N-((1R)-1-(2-pyrimidinyl)ethyl)-1,8-naphthyridine-3-carboxamide,
2-amino-3-bromo-N-((1S)-1-cyclopropyl-2-methoxyethyl)-N-((6-(4-morpholinyl)-3-pyridazinyl)methyl)-6-quinolinecarboxamide,
2-amino-3-bromo-N-((4-chloro-5-cyano-2-pyridinyl)methyl)-N-((1R)-1-(2-pyrimidinyl)ethyl)-6-quinolinecarboxamide,
2-amino-N-((5-cyano-6-methyl-2-pyridinyl)methyl)-3-methyl-N-((1R)-1-(2-pyrimidinyl)ethyl)-6-quinolinecarboxamide,
2-amino-3-bromo-N-((1S)-2-cyano-1-cyclopropylethyl)-N-((5-cyano-2-pyridinyl)methyl)-6-quinolinecarboxamide,
2-amino-N-((4-ethyl-1,3-thiazol-2-yl)methyl)-3-methyl-N-((5-(trifluoromethyl)-2-pyridinyl)methyl)-6-quinolinecarboxamide,
2-amino-3-methyl-N-((1R)-1-((3R)-tetrahydro-3-furanyl)ethyl)-N-((5-(trifluoromethyl)-2-pyridinyl)methyl)-6-quinolinecarboxamide,
2-amino-3-methyl-N-((1R)-1-((3S)-tetrahydro-3-furanyl)ethyl)-N-((5-(trifluoromethyl)-2-pyridinyl)methyl)-6-quinolinecarboxamide,
2-amino-3-methyl-N-((1S)-1-((3R)-tetrahydro-3-furanyl)ethyl)-N-((5-(trifluoromethyl)-2-pyridinyl)methyl)-6-quinolinecarboxamide,
2-amino-3-methyl-N-((1S)-1-((3 S)-tetrahydro-3-furanyl)ethyl)-N-((5-(trifluoromethyl)-2-pyridinyl)methyl)-6-quinolinecarboxamide,
2-amino-3-bromo-N-(3,4-dihydro-2H-pyrano[2,3-c]pyridin-6-ylmethyl)-N-((1R)-1-(2-pyrimidinyl)ethyl)-6-quinolinecarboxamide,
2-amino-3-bromo-N-((6-chloro-3-pyridazinyl)methyl)-N-((1R)-1-(2-pyrimidinyl)ethyl)-6-quinolinecarboxamide,
2-amino-3-bromo-N-((5-cyano-6-methyl-2-pyridinyl)methyl)-N-((1R)-1-(2-pyrimidinyl)ethyl)-6-quinolinecarboxamide,
2-amino-N-((5-cyano-6-methyl-2-pyridinyl)methyl)-3-iodo-N-((1R)-1-(2-pyrimidinyl)ethyl)-6-quinolinecarboxamide,
2-amino-3-bromo-N-((6-(difluoromethoxy)-3-pyridazinyl)methyl)-N-((1R)-1-(2-pyrimidinyl)ethyl)-6-quinolinecarboxamide,
2-amino-3-bromo-N-((1R,2R)-2-cyanocyclopentyl)-N-((5-cyano-2-pyridinyl)methyl)-6-quinolinecarboxamide,
2-amino-3-bromo-N-((1R,2S)-2-cyanocyclopentyl)-N-((5-cyano-2-pyridinyl)methyl)-6-quinolinecarboxamide,
2-amino-3-bromo-N-((1S,2R)-2-cyanocyclopentyl)-N-((5-cyano-2-pyridinyl)methyl)-6-quinolinecarboxamide,
2-amino-3-bromo-N-((1S,2S)-2-cyanocyclopentyl)-N-((5-cyano-2-pyridinyl)methyl)-6-quinolinecarboxamide,
2-amino-3-bromo-N-((1-cyanocyclopropyl)methyl)-N-((5-(trifluoromethyl)-2-pyridinyl)methyl)-6-quinolinecarboxamide,
2-amino-3-bromo-N-((3S,4R)-3-methoxytetrahydro-2H-pyran-4-yl)-N-((5-(trifluoromethyl)-2-pyridinyl)methyl)-6-quinolinecarboxamide,
2-amino-3-bromo-N-((3R,4R)-3-methyltetrahydro-2H-pyran-4-yl)-N-((5-(trifluoromethyl)-2-pyridinyl)methyl)-6-quinolinecarboxamide,
2-amino-3-bromo-N-((3R,4S)-3-methyltetrahydro-2H-pyran-4-yl)-N-((5-(trifluoromethyl)-2-pyridinyl)methyl)-6-quinolinecarboxamide,
2-amino-3-bromo-N-((3S,4R)-3-methyltetrahydro-2H-pyran-4-yl)-N-((5-(trifluoromethyl)-2-pyridinyl)methyl)-6-quinolinecarboxamide,
2-amino-3-bromo-N-((3S,4S)-3-methyltetrahydro-2H-pyran-4-yl)-N-((5-(trifluoromethyl)-2-pyridinyl)methyl)-6-quinolinecarboxamide,
2-amino-3-bromo-N-((1R,2R)-2-methylcyclopentyl)-N-((5-(trifluoromethyl)-2-pyridinyl)methyl)-6-quinolinecarboxamide,
2-amino-3-bromo-N-((1R,2S)-2-methylcyclopentyl)-N-((5-(trifluoromethyl)-2-pyridinyl)methyl)-6-quinolinecarboxamide,
2-amino-3-bromo-N-((1S,2R)-2-methylcyclopentyl)-N-((5-(trifluoromethyl)-2-pyridinyl)methyl)-6-quinolinecarboxamide,
2-amino-3-bromo-N-((1S,2S)-2-methylcyclopentyl)-N-((5-(trifluoromethyl)-2-pyridinyl)methyl)-6-quinolinecarboxamide,
2-amino-3-bromo-N-((2R,3R)-2-cyclopropyltetrahydro-3-furanyl)-N-((5-(trifluoromethyl)-2-pyridinyl)methyl)-6-quinolinecarboxamide,
2-amino-3-bromo-N-((2R,3S)-2-cyclopropyltetrahydro-3-furanyl)-N-((5-(trifluoromethyl)-2-pyridinyl)methyl)-6-quinolinecarboxamide,
2-amino-3-bromo-N-((2S,3R)-2-cyclopropyltetrahydro-3-furanyl)-N-((5-(trifluoromethyl)-2-pyridinyl)methyl)-6-quinolinecarboxamide,
2-amino-3 -bromo-N-((2S,3 S)-2-cyclopropyltetrahydro-3 -furanyl)-N-((5 -(trifluoromethyl)-2-pyridinyl)methyl)-6-quinolinecarboxamide,
2-amino-3-bromo-N-((1S)-2-cyano-1-cyclopropylethyl)-N-((5-(trifluoromethyl)-2-pyridinyl)methyl)-6-quinolinecarboxamide,
2-amino-N-((6-chloro-5-cyano-2-pyridinyl)methyl)-3-iodo-N-((1R)-1-(2-pyrimidinyl)ethyl)-6-quinolinecarboxamide,
2-amino-N-((5-cyano-2-pyridinyl)methyl)-3-iodo-N-((1R)-1-(2-pyrimidinyl)propyl)-6-quinolinecarboxamide,
2-amino-N-(3,4-dihydro-2H-pyrano[2,3-c]pyridin-6-ylmethyl)-3-iodo-N-((1R)-1-(2-pyrimidinyl)ethyl)-6-quinolinecarboxamide,
2-amino-N-((1S)-2-cyano-1-cyclopropylethyl)-N-((5-cyano-2-pyridinyl)methyl)-3-iodo-6-quinolinecarboxamide,
2-amino-N-((6-cyclopropyl-3-pyridazinyl)methyl)-3-iodo-N-((1R)-1-(2-pyrimidinyl)ethyl)-6-quinolinecarboxamide,
2-amino-7-hydroxy-3-methyl-N-(2-methylpropyl)-N-((5-(trifluoromethyl)-2-pyridinyl)methyl)-6-quinolinecarboxamide,
7-amino-6-iodo-N-((1R)-1-(2-pyrimidinyl)ethyl)-N-((6-(trifluoromethyl)-3-pyridazinyl)methyl)-1,8-naphthyridine-3-carboxamide,
7-amino-N-((6-ethoxy-3-pyridazinyl)methyl)-6-iodo-N-((1R)-1-(2-pyrimidinyl)ethyl)-1,8-naphthyridine-3-carboxamide,
2-amino-3-methyl-N-(((3R,4R)-3-methyltetrahydro-2H-pyran-4-yl)methyl)-N-((5-(trifluoromethyl)-2-pyridinyl)methyl)-6-quinolinecarboxamide,
2-amino-3-methyl-N-(((3R,4S)-3-methyltetrahydro-2H-pyran-4-yl)methyl)-N-((5-(trifluoromethyl)-2-pyridinyl)methyl)-6-quinolinecarboxamide,
2-amino-3-methyl-N-(((3S,4S)-3-methyltetrahydro-2H-pyran-4-yl)methyl)-N-((5-(trifluoromethyl)-2-pyridinyl)methyl)-6-quinolinecarboxamide,
2-amino-3-methyl-N-(((3S,4R)-3-methyltetrahydro-2H-pyran-4-yl)methyl)-N-((5-(trifluoromethyl)-2-pyridinyl)methyl)-6-quinolinecarboxamide,
2-amino-3-methyl-N-((4R)-4,5,6,7-tetrahydro-1-benzofuran-4-yl)-N-((5-(trifluoromethyl)-2-pyridinyl)methyl)-6-quinolinecarboxamide,
2-amino-3-methyl-N-((4S)-4,5,6,7-tetrahydro-1-benzofuran-4-yl)-N-((5-(trifluoromethyl)-2-pyridinyl)methyl)-6-quinolinecarboxamide,
2-amino-N-((4R)-2-chloro-5,6-dihydro-4H-cyclopenta[b]thiophen-4-yl)-3-methyl-N-((5-(trifluoromethyl)-2-pyridinyl)methyl)-6-quinolinecarboxamide,
2-amino-N-((4S)-2-chloro-5,6-dihydro-4H-cyclopenta[b]thiophen-4-yl)-3-methyl-N-((5-(trifluoromethyl)-2-pyridinyl)methyl)-6-quinolinecarboxamide,
2-amino-N-((3R)-2,3-dihydro-1-benzofuran-3-yl)-3-methyl-N-((5-(trifluoromethyl)-2-pyridinyl)methyl)-6-quinolinecarboxamide,
2-amino-N-((3S)-2,3-dihydro-1-benzofuran-3-yl)-3-methyl-N-((5-(trifluoromethyl)-2-pyridinyl)methyl)-6-quinolinecarboxamide,
2-amino-3-methyl-N-((2R)-3,3,3-trifluoro-2-methoxypropyl)-N-((5-(trifluoromethyl)-2-pyridinyl)methyl)-6-quinolinecarboxamide,
2-amino-N-((1R,3R)-3-hydroxycyclohexyl)-3-methyl-N-((5-(trifluoromethyl)-2-pyridinyl)methyl)-6-quinolinecarboxamide,
2-amino-N-((1R,3S)-3-hydroxycyclohexyl)-3-methyl-N-((5-(trifluoromethyl)-2-pyridinyl)methyl)-6-quinolinecarboxamide,
2-amino-N-((1S,3R)-3-hydroxycyclohexyl)-3-methyl-N-((5-(trifluoromethyl)-2-pyridinyl)methyl)-6-quinolinecarboxamide,
2-amino-N-((1S,3S)-3-hydroxycyclohexyl)-3-methyl-N-((5-(trifluoromethyl)-2-pyridinyl)methyl)-6-quinolinecarboxamide,
2-amino-N-((5-fluoro-3-pyridinyl)methyl)-3-methyl-N-((5-(trifluoromethyl)-2-pyridinyl)methyl)-6-quinolinecarboxamide,
2-amino-3-methyl-N-((5R)-4,5,6,7-tetrahydro-1H-benzimidazol-5-ylmethyl)-N-((5-(trifluoromethyl)-2-pyridinyl)methyl)-6-quinolinecarboxamide,
2-amino-3-methyl-N-((5S)-4,5,6,7-tetrahydro-1H-benzimidazol-5-ylmethyl)-N-((5-(trifluoromethyl)-2-pyridinyl)methyl)-6-quinolinecarboxamide,
2-amino-3-methyl-N-(1H-pyrrolo[2,3-b]pyridin-5-ylmethyl)-N-((5-(trifluoromethyl)-2-pyridinyl)methyl)-6-quinolinecarboxamide,
2-amino-3-methyl-N-((2-oxo-2,5,6,7-tetrahydro-1H-cyclopenta[b]pyridin-3-yl)methyl)-N-((5-(trifluoromethyl)-2-pyridinyl)methyl)-6-quinolinecarboxamide,
2-amino-N-(3-hydroxybenzyl)-3-methyl-N-((5-(trifluoromethyl)-2-pyridinyl)methyl)-6-quinolinecarboxamide,
2-amino-N-((1R)-2,3-dihydro-1H-inden-1-yl)-3-methyl-N-((5-(trifluoromethyl)-2-pyridinyl)methyl)-6-quinolinecarboxamide,
2-amino-3-methyl-N-((1R)-6-methyl-2,3-dihydro-1H-inden-1-yl)-N-((5-(trifluoromethyl)-2-pyridinyl)methyl)-6-quinolinecarboxamide,
2-amino-3-methyl-N-((1S)-6-methyl-2,3-dihydro-1H-inden-1-yl)-N-((5-(trifluoromethyl)-2-pyridinyl)methyl)-6-quinolinecarboxamide,
2-amino-N-(4-(4-carbamoyl-1-piperidinyl)benzyl)-N-((2R)-2-cyclopropylpropyl)-3-methyl-6-quinolinecarboxamide,
2-amino-N-(4-(4-carbamoyl-1-piperidinyl)benzyl)-N-((2S)-2-cyclopropylpropyl)-3-methyl-6-quinolinecarboxamide,
2-amino-N-((2R)-2-cyclopropylpropyl)-3-methyl-N-((5-(trifluoromethyl)-2-pyridinyl)methyl)-6-quinolinecarboxamide,
2-amino-N-((2S)-2-cyclopropylpropyl)-3-methyl-N-((5-(trifluoromethyl)-2-pyridinyl)methyl)-6-quinolinecarboxamide,
(6R)-2-amino-N-(1H-pyrrolo [2,3-b]pyridin-4-ylmethyl)-N-((5-(trifluoromethyl)-2-pyridinyl)methyl)-5,6,7,8-tetrahydro-6-quinolinecarboxamide,
(6S)-2-amino-N-(1H-pyrrolo[2,3-b]pyridin-4-ylmethyl)-N-((5-(trifluoromethyl)-2-pyridinyl)methyl)-5,6,7,8-tetrahydro-6-quinolinecarboxamide,
2-amino-N-((2R)-2-ethoxypropyl)-3-methyl-N-((5-(trifluoromethyl)-2-pyridinyl)methyl)-6-quinolinecarboxamide,
2-amino-N-((2S)-2-ethoxypropyl)-3-methyl-N-((5-(trifluoromethyl)-2-pyridinyl)methyl)-6-quinolinecarboxamide,
2-amino-N-((1,3-dimethyl-1H-indol-2-yl)methyl)-3-methyl-N-((5-(trifluoromethyl)-2-pyridinyl)methyl)-6-quinolinecarboxamide,
2-amino-N-(2-fluorobenzyl)-N-((5-fluoro-3-pyridinyl)methyl)-3-methyl-6-quinolinecarboxamide,
2-amino-3-methyl-N-((2R)-3,3,3-trifluoro-2-hydroxypropyl)-N-((5-(trifluoromethyl)-2-pyridinyl)methyl)-6-quinolinecarboxamide,
2-amino-3-methyl-N-((2S)-3,3,3-trifluoro-2-hydroxypropyl)-N-((5-(trifluoromethyl)-2-pyridinyl)methyl)-6-quinolinecarboxamide,
2-amino-3-methyl-N-((7S)-4,5,6,7-tetrahydro-1H-indazol-7-yl)-N-((5-(trifluoromethyl)-2-pyridinyl)methyl)-6-quinolinecarboxamide,
7-amino-N-((1R,2R)-2-cyanocyclopentyl)-6-methyl-N-((5-(trifluoromethyl)-2-pyridinyl)methyl)-1,8-naphthyridine-3-carboxamide,
7-amino-N-((1R,2S)-2-cyanocyclopentyl)-6-methyl-N-((5-(trifluoromethyl)-2-pyridinyl)methyl)-1,8-naphthyridine-3-carboxamide,
7-amino-N-((1S,2R)-2-cyanocyclopentyl)-6-methyl-N-((5-(trifluoromethyl)-2-pyridinyl)methyl)-1,8-naphthyridine-3-carboxamide,
7-amino-N-((1S,2S)-2-cyanocyclopentyl)-6-methyl-N-((5-(trifluoromethyl)-2-pyridinyl)methyl)-1,8-naphthyridine-3-carboxamide,
2-amino-N-((1R,2R)-2-cyanocyclopentyl)-3-methyl-N-((5-(trifluoromethyl)-2-pyridinyl)methyl)-1,7-naphthyridine-6-carboxamide,
2-amino-N-((1R,2S)-2-cyanocyclopentyl)-3-methyl-N-((5-(trifluoromethyl)-2-pyridinyl)methyl)-1,7-naphthyridine-6-carboxamide,
2-amino-N-((1S,2R)-2-cyanocyclopentyl)-3-methyl-N-((5-(trifluoromethyl)-2-pyridinyl)methyl)-1,7-naphthyridine-6-carboxamide,
2-amino-N-((1S,2S)-2-cyanocyclopentyl)-3-methyl-N-((5-(trifluoromethyl)-2-pyridinyl)methyl)-1,7-naphthyridine-6-carboxamide,
2-amino-N-((1R,2R)-2-cyanocyclopentyl)-7-fluoro-3-methyl-N-((5-(trifluoromethyl)-2-pyridinyl)methyl)-6-quinolinecarboxamide,
2-amino-N-((1R,2S)-2-cyanocyclopentyl)-7-fluoro-3-methyl-N-((5-(trifluoromethyl)-2-pyridinyl)methyl)-6-quinolinecarboxamide,
2-amino-N-((1S,2R)-2-cyanocyclopentyl)-7-fluoro-3-methyl-N-((5-(trifluoromethyl)-2-pyridinyl)methyl)-6-quinolinecarboxamide,
2-amino-N-((1S,2S)-2-cyanocyclopentyl)-7-fluoro-3-methyl-N-((5-(trifluoromethyl)-2-pyridinyl)methyl)-6-quinolinecarboxamide,
2-amino-N-((1R,2R)-2-cyanocyclopentyl)-N-((5-cyano-2-pyridinyl)methyl)-7-fluoro-3-methyl-6-quinolinecarboxamide,
2-amino-N-((1R,2S)-2-cyanocyclopentyl)-N-((5-cyano-2-pyridinyl)methyl)-7-fluoro-3-methyl-6-quinolinecarboxamide,
2-amino-N-((1S,2R)-2-cyanocyclopentyl)-N-((5-cyano-2-pyridinyl)methyl)-7-fluoro-3-methyl-6-quinolinecarboxamide,
2-amino-N-((1S,2S)-2-cyanocyclopentyl)-N-((5-cyano-2-pyridinyl)methyl)-7-fluoro-3-methyl-6-quinolinecarboxamide,
2-amino-N-((1R,2R)-[1,1′-bi(cyclopropyl)]-2-yl)-3-methyl-N-((5-(trifluoromethyl)-2-pyridinyl)methyl)-6-quinolinecarboxamide,
2-amino-N-((1R,2S)-[1,1′-bi(cyclopropyl)]-2-yl)-3-methyl-N-((5-(trifluoromethyl)-2-pyridinyl)methyl)-6-quinolinecarboxamide,
2-amino-N-((1S,2R)-[1,1′-bi(cyclopropyl)]-2-yl)-3-methyl-N-((5-(trifluoromethyl)-2-pyridinyl)methyl)-6-quinolinecarboxamide,
2-amino-N-((1S,2S)-[1,1′-bi(cyclopropyl)]-2-yl)-3-methyl-N-((5-(trifluoromethyl)-2-pyridinyl)methyl)-6-quinolinecarboxamide,
2-amino-N-((1R)-2,2-dimethylcyclopropyl)-3-methyl-N-((5-(trifluoromethyl)-2-pyridinyl)methyl)-6-quinolinecarboxamide,
2-amino-N-((1S)-2,2-dimethylcyclopropyl)-3-methyl-N-((5-(trifluoromethyl)-2-pyridinyl)methyl)-6-quinolinecarboxamide,
2-amino-N-(4-(4-carbamoyl-1-piperidinyl)benzyl)-3-methyl-N-((4-methyl-1,3-thiazol-5-yl)methyl)-6-quinolinecarboxamide,
2-amino-N-(4-(4-carbamoyl-1-piperidinyl)benzyl)-3-methyl-N-(1H-pyrrolo[2,3-b]pyridin-4-ylmethyl)-6-quinolinecarboxamide,
2-amino-N-((1-cyanocyclopropyl)methyl)-3-methyl-N-(4-(1-piperidinyl)benzyl)-6-quinolinecarboxamide,
2-amino-3-methyl-N-(2-(1,2,4-oxadiazol-3-yl)ethyl)-N-((5-(trifluoromethyl)-2-pyridinyl)methyl)-6-quinolinecarboxamide,
2-amino-N-((1-cyanocyclopropyl)methyl)-3-methyl-N-((5-(trifluoromethyl)-2-pyridinyl)methyl)-6-quinolinecarboxamide,
2-amino-N-(2-cyclobutylethyl)-3-methyl-N-((5-(trifluoromethyl)-2-pyridinyl)methyl)-6-quinolinecarboxamide,
2-amino-N-((4-chloro-1-methyl-1H-pyrrol-2-yl)methyl)-3-methyl-N-((5-(trifluoromethyl)-2-pyridinyl)methyl)-6-quinolinecarboxamide,
2-amino-3-methyl-N-(1H-pyrrol-3-ylmethyl)-N-((5-(trifluoromethyl)-2-pyridinyl)methyl)-6-quinolinecarboxamide,
2-amino-3-methyl-N-(2-(1,2-oxazol-3-yl)ethyl)-N-((5-(trifluoromethyl)-2-pyridinyl)methyl)-6-quinolinecarboxamide,
2-amino-N-(2-fluorobenzyl)-3-methyl-N-(cis-3-(4-(trifluoromethoxy)phenyl)cyclobutyl)-6-quinolinecarboxamide,
2-amino-N-(2-fluorobenzyl)-3-methyl-N-(trans-3-(4-(trifluoromethoxy)phenyl)cyclobutyl)-6-quinolinecarboxamide,
2-amino-N-((3-fluoro-4-pyridinyl)methyl)-3-methyl-N-((2-oxo-2,3-dihydro-1H-benzimidazol-5-yl)methyl)-6-quinolinecarboxamide,
2-amino-3-methyl-N-((2-oxo-2,3-dihydro-1H-benzimidazol-5-yl)methyl)-N-(1H-pyrrolo[2,3-b]pyridin-4-ylmethyl)-6-quinolinecarboxamide,
2-amino-N-((4R)-3,4-dihydro-1H-2-benzopyran-4-yl)-3-methyl-N-((5-(trifluoromethyl)-2-pyridinyl)methyl)-6-quinolinecarboxamide,
2-amino-N-((4S)-3,4-dihydro-1H-2-benzopyran-4-yl)-3-methyl-N-((5-(trifluoromethyl)-2-pyridinyl)methyl)-6-quinolinecarboxamide,
2-amino-N-((1R)-3,3-dimethyl-2,3-dihydro-1H-inden-1-yl)-3-methyl-N-((5-(trifluoromethyl)-2-pyridinyl)methyl)-6-quinolinecarboxamide,
2-amino-N-((1S)-3,3-dimethyl-2,3-dihydro-1H-inden-1-yl)-3-methyl-N-((5-(trifluoromethyl)-2-pyridinyl)methyl)-6-quinolinecarboxamide,
2-amino-3-methyl-N-((1R,3R)-3-methyl-2,3-dihydro-1H-inden-1-yl)-N-((5-(trifluoromethyl)-2-pyridinyl)methyl)-6-quinolinecarboxamide,
2-amino-3-methyl-N-((1R,3 S)-3-methyl-2,3-dihydro-1H-inden-1-yl)-N-((5-(trifluoromethyl)-2-pyridinyl)methyl)-6-quinolinecarboxamide,
2-amino-3-methyl-N-((1S,3R)-3-methyl-2,3-dihydro-1H-inden-1-yl)-N-((5-(trifluoromethyl)-2-pyridinyl)methyl)-6-quinolinecarboxamide,
2-amino-3-methyl-N-((1S,3S)-3-methyl-2,3-dihydro-1H-inden-1-yl)-N-((5-(trifluoromethyl)-2-pyridinyl)methyl)-6-quinolinecarboxamide,
2-amino-3-methyl-N-((1R,4R)-4-methyl-1,2,3,4-tetrahydro-1-naphthalenyl)-N-((5-(trifluoromethyl)-2-pyridinyl)methyl)-6-quinolinecarboxamide,
2-amino-3-methyl-N-((1R,4S)-4-methyl-1,2,3,4-tetrahydro-1-naphthalenyl)-N-((5-(trifluoromethyl)-2-pyridinyl)methyl)-6-quinolinecarboxamide,
2-amino-3-methyl-N-((1S,4R)-4-methyl-1,2,3,4-tetrahydro-1-naphthalenyl)-N-((5-(trifluoromethyl)-2-pyridinyl)methyl)-6-quinolinecarboxamide,
2-amino-3-methyl-N-((1S,4S)-4-methyl-1,2,3,4-tetrahydro-1-naphthalenyl)-N-((5-(trifluoromethyl)-2-pyridinyl)methyl)-6-quinolinecarboxamide,
2-amino-N-((4R)-8-methoxy-3,4-dihydro-2H-chromen-4-yl)-3-methyl-N-((5-(trifluoromethyl)-2-pyridinyl)methyl)-6-quinolinecarboxamide,
2-amino-N-((4S)-8-methoxy-3,4-dihydro-2H-chromen-4-yl)-3-methyl-N-((5-(trifluoromethyl)-2-pyridinyl)methyl)-6-quinolinecarboxamide,
2-amino-3-methyl-N-((4R)-4,5,6,7-tetrahydro-1H-indol-4-yl)-N-((5-(trifluoromethyl)-2-pyridinyl)methyl)-6-quinolinecarboxamide,
2-amino-3-methyl-N-((4S)-4,5,6,7-tetrahydro-1H-indol-4-yl)-N-((5-(trifluoromethyl)-2-pyridinyl)methyl)-6-quinolinecarboxamide,
2-amino-3-methyl-N-((7R)-2-methyl-4,5,6,7-tetrahydro-1,3-benzothiazol-7-yl)-N-((5-(trifluoromethyl)-2-pyridinyl)methyl)-6-quinolinecarboxamide,
2-amino-3-methyl-N-((7S)-2-methyl-4,5,6,7-tetrahydro-1,3-benzothiazol-7-yl)-N-((5-(trifluoromethyl)-2-pyridinyl)methyl)-6-quinolinecarboxamide,
2-amino-3-methyl-N-((4R)-2-methyl-4,5,6,7-tetrahydro-2H-indazol-4-yl)-N-((5-(trifluoromethyl)-2-pyridinyl)methyl)-6-quinolinecarboxamide,
2-amino-3-methyl-N-((4S)-2-methyl-4,5,6,7-tetrahydro-2H-indazol-4-yl)-N-((5-(trifluoromethyl)-2-pyridinyl)methyl)-6-quinolinecarboxamide,
2-amino-N-((1R,2R)-2-hydroxy-2,3-dihydro-1H-inden-1-yl)-3-methyl-N-((5-(trifluoromethyl)-2-pyridinyl)methyl)-6-quinolinecarboxamide,
2-amino-N-((3R)-6,7-dimethyl-2,3-dihydro-1-benzofuran-3-yl)-3-methyl-N-((5-(trifluoromethyl)-2-pyridinyl)methyl)-6-quinolinecarboxamide,
2-amino-N-((3 S)-6, 7-dimethyl-2,3-dihydro-1-benzofuran-3-yl)-3-methyl-N-((5-(trifluoromethyl)-2-pyridinyl)methyl)-6-quinolinecarboxamide,
2-amino-N-((2R)-2-methoxy-3-methylbutyl)-3-methyl-N-((5-(trifluoromethyl)-2-pyridinyl)methyl)-6-quinolinecarboxamide,
2-amino-N-((2S)-2-methoxy-3-methylbutyl)-3-methyl-N-((5-(trifluoromethyl)-2-pyridinyl)methyl)-6-quinolinecarboxamide,
2-amino-3-methyl-N-((7R)-5,6,7,8-tetrahydroimidazo[1,2-a]pyridin-7-ylmethyl)-N-((5-(trifluoromethyl)-2-pyridinyl)methyl)-6-quinolinecarboxamide,
2-amino-3-methyl-N-((7S)-5,6,7,8-tetrahydroimidazo[1,2-a]pyridin-7-ylmethyl)-N-((5-(trifluoromethyl)-2-pyridinyl)methyl)-6-quinolinecarboxamide,
2-amino-3-methyl-N-(1H-pyrrolo[2,3-b]pyridin-6-ylmethyl)-N-((5-(trifluoromethyl)-2-pyridinyl)methyl)-6-quinolinecarboxamide,
2-amino-N-((2-amino-1H-benzimidazol-5-yl)methyl)-3-methyl-N-((5-(trifluoromethyl)-2-pyridinyl)methyl)-6-quinolinecarboxamide,
2-amino-N-(1H-indazol-6-ylmethyl)-3-methyl-N-((5-(trifluoromethyl)-2-pyridinyl)methyl)-6-quinolinecarboxamide,
2-amino-3-methyl-N-((2-oxo-2,3-dihydro-1,3-benzoxazol-6-yl)methyl)-N-((5-(trifluoromethyl)-2-pyridinyl)methyl)-6-quinolinecarboxamide,
2-amino-N-(imidazo[1,2-a]pyridin-7-ylmethyl)-3-methyl-N-((5-(trifluoromethyl)-2-pyridinyl)methyl)-6-quinolinecarboxamide,
2-amino-N-(1-benzofuran-6-ylmethyl)-3-methyl-N-((5-(trifluoromethyl)-2-pyridinyl)methyl)-6-quinolinecarboxamide,
2-amino-N-(1H-indol-5-ylmethyl)-3-methyl-N-((5-(trifluoromethyl)-2-pyridinyl)methyl)-6-quinolinecarboxamide,
2-amino-3-methyl-N-((1R)-1-(3-oxo-3,4-dihydro-2H-1,4-benzoxazin-6-yl)ethyl)-N-((5-(trifluoromethyl)-2-pyridinyl)methyl)-6-quinolinecarboxamide,
2-amino-3-methyl-N-((1S)-1-(3-oxo-3,4-dihydro-2H-1,4-benzoxazin-6-yl)ethyl)-N-((5-(trifluoromethyl)-2-pyridinyl)methyl)-6-quinolinecarboxamide,
2-amino-3-methyl-N-(5-pyrimidinylmethyl)-N-((5-(trifluoromethyl)-2-pyridinyl)methyl)-6-quinolinecarboxamide,
2-amino-3-methyl-N-((2-oxo-1,2-dihydro-3-pyridinyl)methyl)-N-((5-(trifluoromethyl)-2-pyridinyl)methyl)-6-quinolinecarboxamide,
2-amino-N-((1R)-4-hydroxy-2,3-dihydro-1H-inden-1-yl)-3-methyl-N-((5-(trifluoromethyl)-2-pyridinyl)methyl)-6-quinolinecarboxamide,
2-amino-N-((1S)-4-hydroxy-2,3-dihydro-1H-inden-1-yl)-3-methyl-N-((5-(trifluoromethyl)-2-pyridinyl)methyl)-6-quinolinecarboxamide,
2-amino-3-methyl-N-((3aR,4R,7aR)-octahydro-1-benzofuran-4-yl)-N-((5-(trifluoromethyl)-2-pyridinyl)methyl)-6-quinolinecarboxamide,
2-amino-3-methyl-N-((3aR,4R,7aS)-octahydro-1-benzofuran-4-yl)-N-((5-(trifluoromethyl)-2-pyridinyl)methyl)-6-quinolinecarboxamide,
2-amino-3-methyl-N-((3aR,4S,7aR)-octahydro-1-benzofuran-4-yl)-N-((5-(trifluoromethyl)-2-pyridinyl)methyl)-6-quinolinecarboxamide,
2-amino-3-methyl-N-((3aR,4S,7aS)-octahydro-1-benzofuran-4-yl)-N-((5-(trifluoromethyl)-2-pyridinyl)methyl)-6-quinolinecarboxamide,
2-amino-3-methyl-N-((3aS,4R,7aR)-octahydro-1-benzofuran-4-yl)-N-((5-(trifluoromethyl)-2-pyridinyl)methyl)-6-quinolinecarboxamide,
2-amino-3-methyl-N-((3aS,4R,7aS)-octahydro-1-benzofuran-4-yl)-N-((5-(trifluoromethyl)-2-pyridinyl)methyl)-6-quinolinecarboxamide,
2-amino-3-methyl-N-((3aS,4S,7aR)-octahydro-1-benzofuran-4-yl)-N-((5-(trifluoromethyl)-2-pyridinyl)methyl)-6-quinolinecarboxamide,
2-amino-3-methyl-N-((3aS,4S,7aS)-octahydro-1-benzofuran-4-yl)-N-((5-(trifluoromethyl)-2-pyridinyl)methyl)-6-quinolinecarboxamide,
2-amino-3-methyl-N-((2R)-4-methyl-2-pentanyl)-N-((5-(trifluoromethyl)-2-pyridinyl)methyl)-6-quinolinecarboxamide,
2-amino-3-methyl-N-((2S)-4-methyl-2-pentanyl)-N-((5-(trifluoromethyl)-2-pyridinyl)methyl)-6-quinolinecarboxamide,
2-amino-3-methyl-N-((1-oxo-2,3-dihydro-1H-isoindol-5-yl)methyl)-N-((5-(trifluoromethyl)-2-pyridinyl)methyl)-6-quinolinecarboxamide,
2-amino-N-((6-carbamoyl-3-pyridinyl)methyl)-3-methyl-N-((5-(trifluoromethyl)-2-pyridinyl)methyl)-6-quinolinecarboxamide,
2-amino-3-methyl-N-(4-(methylcarbamoyl)benzyl)-N-((5-(trifluoromethyl)-2-pyridinyl)methyl)-6-quinolinecarboxamide,
2-amino-N-((4-chloro-2-pyridinyl)methyl)-3-methyl-N-(2-methylpropyl)-6-quinolinecarboxamide,
2-amino-N-((3-bromo-2-pyridinyl)methyl)-3-methyl-N-(2-methylpropyl)-6-quinolinecarboxamide,
2-amino-N-((2R,3R)-3-hydroxy-2-butanyl)-3-methyl-N-((5-(trifluoromethyl)-2-pyridinyl)methyl)-6-quinolinecarboxamide,
2-amino-N-((2R,3S)-3-hydroxy-2-butanyl)-3-methyl-N-((5-(trifluoromethyl)-2-pyridinyl)methyl)-6-quinolinecarboxamide,
2-amino-N-((2S,3R)-3-hydroxy-2-butanyl)-3-methyl-N-((5-(trifluoromethyl)-2-pyridinyl)methyl)-6-quinolinecarboxamide,
2-amino-N-((2S,3S)-3-hydroxy-2-butanyl)-3-methyl-N-((5-(trifluoromethyl)-2-pyridinyl)methyl)-6-quinolinecarboxamide,
2-amino-3-methyl-N-((2R)-3-methyl-2-butanyl)-N-((5-(trifluoromethyl)-2-pyridinyl)methyl)-6-quinolinecarboxamide,
2-amino-3-methyl-N-((2S)-3-methyl-2-butanyl)-N-((5-(trifluoromethyl)-2-pyridinyl)methyl)-6-quinolinecarboxamide,
2-amino-3-methyl-N-(2-methylpropyl)-N-(4-(1-pyrrolidinyl)benzyl)-6-quinolinecarboxamide,
2-amino-3-methyl-N-(2-methylpropyl)-N-(3-(1H-1,2,4-triazol-1-yl)benzyl)-6-quinolinecarboxamide,
2-amino-3-methyl-N-(4-(2-oxo-1-pyrrolidinyl)benzyl)-N-((5-(trifluoromethyl)-2-pyridinyl)methyl)-6-quinolinecarboxamide,
2-amino-3-methyl-N-(4-(1-pyrrolidinyl)benzyl)-N-((5-(trifluoromethyl)-2-pyridinyl)methyl)-6-quinolinecarboxamide,
2-amino-3-methyl-N-(4-(1H-pyrazol-3-yl)benzyl)-N-((5-(trifluoromethyl)-2-pyridinyl)methyl)-6-quinolinecarboxamide,
2-amino-N-(3-(1H-imidazol-1-yl)benzyl)-3-methyl-N-((5-(trifluoromethyl)-2-pyridinyl)methyl)-6-quinolinecarboxamide,
2-amino-3-methyl-N-(3-(1H-1,2,4-triazol-1-yl)benzyl)-N-((5-(trifluoromethyl)-2-pyridinyl)methyl)-6-quinolinecarboxamide,
2-amino-N-(3-(1H-imidazol-2-yl)benzyl)-3-methyl-N-((5-(trifluoromethyl)-2-pyridinyl)methyl)-6-quinolinecarboxamide,
2-amino-3-methyl-N-(3-(1H-1,2,3-triazol-1-yl)benzyl)-N-((5-(trifluoromethyl)-2-pyridinyl)methyl)-6-quinolinecarboxamide,
2-amino-3-methyl-N-((2-(1H-1,2,4-triazol-1-yl)-3-pyridinyl)methyl)-N-((5-(trifluoromethyl)-2-pyridinyl)methyl)-6-quinolinecarboxamide,
2-amino-N-(2-(1H-imidazol-1-yl)benzyl)-3-methyl-N-((5-(trifluoromethyl)-2-pyridinyl)methyl)-6-quinolinecarboxamide,
2-amino-3-methyl-N-(2-(1H-pyrazol-1-yl)benzyl)-N-((5-(trifluoromethyl)-2-pyridinyl)methyl)-6-quinolinecarboxamide,
2-amino-3-methyl-N-(2-propyn-1-yl)-N-((5-(trifluoromethyl)-2-pyridinyl)methyl)-6-quinolinecarboxamide,
2-amino-N-((2R)-3-butyn-2-yl)-3-methyl-N-((5-(trifluoromethyl)-2-pyridinyl)methyl)-6-quinolinecarboxamide,
2-amino-N-((2S)-3-butyn-2-yl)-3-methyl-N-((5-(trifluoromethyl)-2-pyridinyl)methyl)-6-quinolinecarboxamide,
2-amino-N-(2-butyn-1-yl)-3-methyl-N-((5-(trifluoromethyl)-2-pyridinyl)methyl)-6-quinolinecarboxamide,
2-amino-N-(4-hydroxy-2-butyn-1-yl)-3-methyl-N-((5-(trifluoromethyl)-2-pyridinyl)methyl)-6-quinolinecarboxamide,
2-amino-N-(3-butyn-1-yl)-3-methyl-N-((5-(trifluoromethyl)-2-pyridinyl)methyl)-6-quinolinecarboxamide,
2-amino-3-methyl-N-(4-((1R)-2,2,2-trifluoro-1-hydroxyethyl)benzyl)-N-((5-(trifluoromethyl)-2-pyridinyl)methyl)-6-quinolinecarboxamide,
2-amino-3-methyl-N-(4-((1S)-2,2,2-trifluoro-1-hydroxyethyl)benzyl)-N-((5-(trifluoromethyl)-2-pyridinyl)methyl)-6-quinolinecarboxamide,
2-amino-N-(4-hydroxybenzyl)-3-methyl-N-((5-(trifluoromethyl)-2-pyridinyl)methyl)-6-quinolinecarboxamide,
2-amino-N-(3-fluoro-4-hydroxybenzyl)-3-methyl-N-((5-(trifluoromethyl)-2-pyridinyl)methyl)-6-quinolinecarboxamide,
2-amino-N-((1R)-1-(3-hydroxyphenyl)ethyl)-3-methyl-N-((5-(trifluoromethyl)-2-pyridinyl)methyl)-6-quinolinecarboxamide,
2-amino-N-((1S)-1-(3-hydroxyphenyl)ethyl)-3-methyl-N-((5-(trifluoromethyl)-2-pyridinyl)methyl)-6-quinolinecarboxamide,
2-amino-3-methyl-N-((1R)-1-(2-oxo-1,2-dihydro-4-pyridinyl)ethyl)-N-((5-(trifluoromethyl)-2-pyridinyl)methyl)-6-quinolinecarboxamide,
2-amino-3-methyl-N-((1S)-1-(2-oxo-1,2-dihydro-4-pyridinyl)ethyl)-N-((5-(trifluoromethyl)-2-pyridinyl)methyl)-6-quinolinecarboxamide,
2-amino-N-((4-bromo-2-pyridinyl)methyl)-3-methyl-N-(2-methylpropyl)-6-quinolinecarboxamide,
2-amino-N-((8R)-4-methoxy-5,6,7,8-tetrahydro-8-quinolinyl)-3-methyl-N-((5-(trifluoromethyl)-2-pyridinyl)methyl)-6-quinolinecarboxamide,
2-amino-N-((8S)-4-methoxy-5,6,7,8-tetrahydro-8-quinolinyl)-3-methyl-N-((5-(trifluoromethyl)-2-pyridinyl)methyl)-6-quinolinecarboxamide,
2-amino-3-methyl-N-((5R)-5,6,7,8-tetrahydro-5-quinolinyl)-N-((5-(trifluoromethyl)-2-pyridinyl)methyl)-6-quinolinecarboxamide,
2-amino-3-methyl-N-((5S)-5,6,7,8-tetrahydro-5-quinolinyl)-N-((5-(trifluoromethyl)-2-pyridinyl)methyl)-6-quinolinecarboxamide,
2-amino-N-((6R)-2-amino-5,6,7,8-tetrahydro-6-quinazolinyl)-3-methyl-N-((5-(trifluoromethyl)-2-pyridinyl)methyl)-6-quinolinecarboxamide,
2-amino-N-((6S)-2-amino-5,6,7,8-tetrahydro-6-quinazolinyl)-3-methyl-N-((5-(trifluoromethyl)-2-pyridinyl)methyl)-6-quinolinecarboxamide,
2-amino-3-methyl-N-((3aR,5R,7aR)-octahydro-1H-inden-5-yl)-N-((5-(trifluoromethyl)-2-pyridinyl)methyl)-6-quinolinecarboxamide,
2-amino-3-methyl-N-((3aR,5R,7aS)-octahydro-1H-inden-5-yl)-N-((5-(trifluoromethyl)-2-pyridinyl)methyl)-6-quinolinecarboxamide,
2-amino-3-methyl-N-((3aR,5S,7aR)-octahydro-1H-inden-5-yl)-N-((5-(trifluoromethyl)-2-pyridinyl)methyl)-6-quinolinecarboxamide,
2-amino-3-methyl-N-((3aR,5S,7aS)-octahydro-1H-inden-5-yl)-N-((5-(trifluoromethyl)-2-pyridinyl)methyl)-6-quinolinecarboxamide,
2-amino-3-methyl-N-((3aS,5R,7aR)-octahydro-1H-inden-5-yl)-N-((5-(trifluoromethyl)-2-pyridinyl)methyl)-6-quinolinecarboxamide,
2-amino-3-methyl-N-((3aS,5R,7aS)-octahydro-1H-inden-5-yl)-N-((5-(trifluoromethyl)-2-pyridinyl)methyl)-6-quinolinecarboxamide,
2-amino-3-methyl-N-((3aS,5S,7aR)-octahydro-1H-inden-5-yl)-N-((5-(trifluoromethyl)-2-pyridinyl)methyl)-6-quinolinecarboxamide,
2-amino-3-methyl-N-((3aS,5S,7aS)-octahydro-1H-inden-5-yl)-N-((5-(trifluoromethyl)-2-pyridinyl)methyl)-6-quinolinecarboxamide,
2-amino-3-methyl-N-((5R)-4,5,6,7-tetrahydro-1H-benzimidazol-5-yl)-N-((5-(trifluoromethyl)-2-pyridinyl)methyl)-6-quinolinecarboxamide,
2-amino-3-methyl-N-((5S)-4,5,6,7-tetrahydro-1H-benzimidazol-5-yl)-N-((5-(trifluoromethyl)-2-pyridinyl)methyl)-6-quinolinecarboxamide,
2-amino-3-methyl-N-((6R)-4,5,6,7-tetrahydro-1H-indazol-6-yl)-N-((5-(trifluoromethyl)-2-pyridinyl)methyl)-6-quinolinecarboxamide,
2-amino-3-methyl-N-((6S)-4,5,6,7-tetrahydro-1H-indazol-6-yl)-N-((5-(trifluoromethyl)-2-pyridinyl)methyl)-6-quinolinecarboxamide,
2-amino-3-methyl-N-((5R)-4,5,6,7-tetrahydro-1H-indazol-5-yl)-N-((5-(trifluoromethyl)-2-pyridinyl)methyl)-6-quinolinecarboxamide,
2-amino-3-methyl-N-((5 S)-4,5,6,7-tetrahydro-1H-indazol-5-yl)-N-((5-(trifluoromethyl)-2-pyridinyl)methyl)-6-quinolinecarboxamide,
2-amino-N-((4R)-1-ethyl-4,5,6,7-tetrahydro-1H-indazol-4-yl)-3-methyl-N-((5-(trifluoromethyl)-2-pyridinyl)methyl)-6-quinolinecarboxamide,
2-amino-N-((4S)-1-ethyl-4,5,6,7-tetrahydro-1H-indazol-4-yl)-3-methyl-N-((5-(trifluoromethyl)-2-pyridinyl)methyl)-6-quinolinecarboxamide,
2-amino-N-((1R,2R)-1-hydroay-2,3-dihydro-1H-inden-2-yl)-3-methyl-N-((5-(trifluoromethyl)-2-pyridinyl)methyl)-6-quinolinecarboxamide,
2-amino-N-((1R,2S)-1-hydroxy-2,3-dihydro-1H-inden-2-yl)-3-methyl-N-((5-(trifluoromethyl)-2-pyridinyl)methyl)-6-quinolinecarboxamide,
2-amino-N-((1S,2R)-1-hydroxy-2,3-dihydro-1H-inden-2-yl)-3-methyl-N-((5-(trifluoromethyl)-2-pyridinyl)methyl)-6-quinolinecarboxamide,
2-amino-N-((1S,2S)-1-hydroxy-2,3-dihydro-1H-inden-2-yl)-3-methyl-N-((5-(trifluoromethyl)-2-pyridinyl)methyl)-6-quinolinecarboxamide,
2-amino-3-methyl-N-((1R)-1-(tetrahydro-2H-pyran-4-yl)ethyl)-N-((5-(trifluoromethyl)-2-pyridinyl)methyl)-6-quinolinecarboxamide,
2-amino-3-methyl-N-((1S)-1-(tetrahydro-2H-pyran-4-yl)ethyl)-N-((5-(trifluoromethyl)-2-pyridinyl)methyl)-6-quinolinecarboxamide,
2-amino-N-((trans-4-hydroxycyclohexyl)methyl)-3-methyl-N-((5-(trifluoromethyl)-2-pyridinyl)methyl)-6-quinolinecarboxamide,
2-amino-N-(((1R,3R)-3-hydroxycyclopentyl)methyl)-3-methyl-N-((5-(trifluoromethyl)-2-pyridinyl)methyl)-6-quinolinecarboxamide,
2-amino-N-(((1R,3S)-3-hydroxycyclopentyl)methyl)-3-methyl-N-((5-(trifluoromethyl)-2-pyridinyl)methyl)-6-quinolinecarboxamide,
2-amino-N-(((1S,3R)-3-hydroxycyclopentyl)methyl)-3-methyl-N-((5-(trifluoromethyl)-2-pyridinyl)methyl)-6-quinolinecarboxamide,
2-amino-N-(((1S,3S)-3-hydroxycyclopentyl)methyl)-3-methyl-N-((5-(trifluoromethyl)-2-pyridinyl)methyl)-6-quinolinecarboxamide,
2-amino-N-(((1R,3R)-3-hydroxycyclohexyl)methyl)-3-methyl-N-((5-(trifluoromethyl)-2-pyridinyl)methyl)-6-quinolinecarboxamide,
2-amino-N-(((1R,3S)-3-hydroxycyclohexyl)methyl)-3-methyl-N-((5-(trifluoromethyl)-2-pyridinyl)methyl)-6-quinolinecarboxamide,
2-amino-N-(((1S,3R)-3-hydroxycyclohexyl)methyl)-3-methyl-N-((5-(trifluoromethyl)-2-pyridinyl)methyl)-6-quinolinecarboxamide,
2-amino-N-(((1S,3S)-3-hydroxycyclohexyl)methyl)-3-methyl-N-((5-(trifluoromethyl)-2-pyridinyl)methyl)-6-quinolinecarboxamide,
2-amino-3-methyl-N-((2R,3R)-3-(1H-pyrazol-1-yl)-2-butanyl)-N-((5-(trifluoromethyl)-2-pyridinyl)methyl)-6-quinolinecarboxamide,
2-amino-3-methyl-N-((2R,3S)-3-(1H-pyrazol-1-yl)-2-butanyl)-N-((5-(trifluoromethyl)-2-pyridinyl)methyl)-6-quinolinecarboxamide,
2-amino-3-methyl-N-((2S,3R)-3-(1H-pyrazol-1-yl)-2-butanyl)-N-((5-(trifluoromethyl)-2-pyridinyl)methyl)-6-quinolinecarboxamide,
2-amino-3-methyl-N-((2S,3S)-3-(1H-pyrazol-1-yl)-2-butanyl)-N-((5-(trifluoromethyl)-2-pyridinyl)methyl)-6-quinolinecarboxamide,
2-amino-N-(4-(2-amino-2-oxoethyl)benzyl)-3-methyl-N-((5-(trifluoromethyl)-2-pyridinyl)methyl)-6-quinolinecarboxamide,
2-amino-3-methyl-N-((1R)-1-(tetrahydro-2H-pyran-4-yl)propyl)-N-((5-(trifluoromethyl)-2-pyridinyl)methyl)-6-quinolinecarboxamide,
2-amino-3-methyl-N-((1S)-1-(tetrahydro-2H-pyran-4-yl)propyl)-N-((5-(trifluoromethyl)-2-pyridinyl)methyl)-6-quinolinecarboxamide,
2-amino-N-((1R)-3-amino-3-oxo-1-(tetrahydro-2H-pyran-4-yl)propyl)-3-methyl-N-((5-(trifluoromethyl)-2-pyridinyl)methyl)-6-quinolinecarboxamide,
2-amino-N-((1S)-3-amino-3-oxo-1-(tetrahydro-2H-pyran-4-yl)propyl)-3-methyl-N-((5-(trifluoromethyl)-2-pyridinyl)methyl)-6-quinolinecarboxamide,
2-amino-3-methyl-N-(tetrahydro-2H-pyran-4-ylmethyl)-N-((5-(trifluoromethyl)-2-pyridinyl)methyl)-6-quinolinecarboxamide,
2-amino-3-methyl-N-(1H-pyrrolo[2,3-b]pyridin-4-ylmethyl)-N-((5-(trifluoromethyl)-2-pyridinyl)methyl)-1,7-naphthyridine-6-carboxamide,
2-amino-N-((3,5-difluoro-4-pyridinyl)methyl)-3-methyl-N-(4-(trifluoromethyl)benzyl)-6-quinolinecarboxamide,
2-amino-3-methyl-N-((1-methyl-1H-pyrazol-5-yl)methyl)-N-((5-(trifluoromethyl)-2-pyridinyl)methyl)-6-quinolinecarboxamide,
2-amino-3-methyl-N-((1-methyl-1H-pyrazol-5-yl)methyl)-N-(1H-pyrrolo[2,3-b]pyridin-4-ylmethyl)-6-quinolinecarboxamide,
2-amino-N-(4-hydroxybenzyl)-3-methyl-N-(1H-pyrrolo[2,3-b]pyridin-4-ylmethyl)-6-quinolinecarboxamide,
2-amino-3-methyl-N-(1H-pyrrolo[2,3-b]pyridin-4-ylmethyl)-N-(1H-pyrrol-2-ylmethyl)-6-quinolinecarboxamide,
2-amino-N-(2-hydroxybenzyl)-3-methyl-N-(1H-pyrrolo[2,3-b]pyridin-4-ylmethyl)-6-quinolinecarboxamide,
2-amino-N-((5-amino-2-pyridinyl)methyl)-N-(1H-indol-3-ylmethyl)-3-methyl-6-quinolinecarboxamide,
2-amino-3-methyl-N-(4-pentyn-1-yl)-N-((5-(trifluoromethyl)-2-pyridinyl)methyl)-6-quinolinecarboxamide,
2-amino-N-((2R)-1-cyano-2-propanyl)-3-methyl-N-((5-(trifluoromethyl)-2-pyridinyl)methyl)-6-quinolinecarboxamide,
2-amino-N-((2S)-1-cyano-2-propanyl)-3-methyl-N-((5-(trifluoromethyl)-2-pyridinyl)methyl)-6-quinolinecarboxamide,
2-amino-N-((2R)-2-cyanopropyl)-3-methyl-N-((5-(trifluoromethyl)-2-pyridinyl)methyl)-6-quinolinecarboxamide,
2-amino-N-((2S)-2-cyanopropyl)-3-methyl-N-((5-(trifluoromethyl)-2-pyridinyl)methyl)-6-quinolinecarboxamide,
2-amino-N-(2-cyclopropylethyl)-3-methyl-N-((5-(trifluoromethyl)-2-pyridinyl)methyl)-6-quinolinecarboxamide,
2-amino-N-((2R)-1-cyclopropyl-2-propanyl)-3-methyl-N-((5-(trifluoromethyl)-2-pyridinyl)methyl)-6-quinolinecarboxamide,
2-amino-N-((2S)-1-cyclopropyl-2-propanyl)-3-methyl-N-((5-(trifluoromethyl)-2-pyridinyl)methyl)-6-quinolinecarboxamide,
2-amino-N-(cis-3-cyanocyclobutyl)-3-methyl-N-((5-(trifluoromethyl)-2-pyridinyl)methyl)-6-quinolinecarboxamide,
2-amino-N-(trans-3-cyanocyclobutyl)-3-methyl-N-((5-(trifluoromethyl)-2-pyridinyl)methyl)-6-quinolinecarboxamide and
2-amino-N-(6,6-difluorospiro[3.3]heptan-2-yl)-3-methyl-N-((5-(trifluoromethyl)-2-pyridinyl)methyl)-6-quinolinecarboxamide.

The inenvention further provides methods of treating cancer comprising administering to a subject an effective amount of the compound of the invention, the tautomer thereof, the stereoisomer thereof, or the pharmaceutically acceptable salt of any of the foregoing. In one aspect, the cancer is selected from ovarian, lung, lymphoid, glioblastoma, colon, melanoma, gastric, pancreatic or bladder cancer.

The invention further provides pharmaceutical compositions, comprising the compounds of the inveniton, the tautomer thereof, the stereoisomer thereof, or the pharmaceutically acceptable salt of any of the foregoing or a pharmaceutically acceptable salt thereof, and at least one pharmaceutically acceptable excipient.

The invention also provides methods of treating a cancer, the method comprising administering to a subject an effective amount of the compound of the invention, the tautomer thereof, the stereoisomer thereof, or the pharmaceutically acceptable salt of any of the foregoing. In one aspect, the cancer can be ovarian, lung, lymphoid, glioblastoma, colon, melanoma, gastric, pancreatic or bladder cancer.

Other objects, features and advantages of the invention will become apparent to those skilled in the art from the following description and claims.

### DETAILED DESCRIPTION OF THE INVENTION

### Definitions

As used herein, if any variable occurs more than one time in a chemical formula, its definition on each occurrence is independent of its definition at every other occurrence. If the chemical structure and chemical name conflict, the chemical structure is determinative of the identity of the compound. The compounds of the present disclosure may contain one or more chiral centers and/or double bonds and therefore, may exist as stereoisomers, such as double-bond isomers (i.e., geometric isomers), enantiomers, or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Certain compounds of the invention may possess asymmetric carbon atoms (optical centers) or double bonds; the racemates, enantiomers, diastereomers, geometric isomers and individual isomers are all intended to be encompassed within the scope of the invention. Furthermore, atropisomers and mixtures thereof such as those resulting from restricted rotation about two aromatic or heteroaromatic rings bonded to one another are intended to be encompassed within the scope of the invention. For example, when substituent is a phenyl group and is substituted with two groups bonded to the C atoms adjacent to the point of attachment to the N atom of the triazole, then rotation of the phenyl may be restricted. In some instances, the barrier of rotation is high enough that the different atropisomers may be separated and isolated.

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the mirror image enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. If the stereochemistry of a structure or a portion of a structure is not indicated with, for example, bold or dashed lines, the structure or portion of the structure is to be interpreted as encompassing all stereoisomers of it. A bond drawn with a wavy line indicates that both stereoisomers are encompassed. This is not to be confused with a wavy line drawn perpendicular to a bond which indicates the point of attachment of a group to the rest of the molecule.

As known by those skilled in the art, certain compounds of the invention may exist in one or more tautomeric forms. Because one chemical structure may only be used to represent one tautomeric form, it will be understood that for convenience, referral to a compound of a given structural formula includes tautomers of the structure represented by the structural formula. Depending on the compound, some compounds may exist primarily in one form more than another. Also, depending on the compound and the energy required to convert one tautomer to the other, some compounds may exist as mixtures at room temperature whereas others may be isolated in one tautomeric form or the other. Examples of other tautomers associated with compounds of the invention are those with a pyridone group (a pyridinyl) for which hydroxypyridine is a tautomer and compounds with a ketone group with the enol tautomer. Examples of these are shown below.

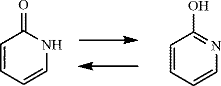 [SMILES: O=c1cccc[nH]1 ; Oc1ccccn1]

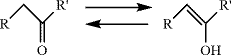

Compounds of the present disclosure include, but are not limited to, compounds of Formula I and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. As used herein, the term “compound” encompasses not only the compound itself, but also a pharmaceutically acceptable salt thereof, a solvate thereof, a chelate thereof, a non-covalent complex thereof, a prodrug thereof, and mixtures of any of the foregoing. In some embodiments, the term “compound” encompasses the compound itself, pharmaceutically acceptable salts thereof, tautomers of the compound, pharmaceutically acceptable salts of the tautomers, and ester prodrugs such as (C₁-C₄)alkyl esters. In other embodiments, the term “compound” encompasses the compound itself, pharmaceutically acceptable salts thereof, tautomers of the compound, pharmaceutically acceptable salts of the tautomers.

The term “solvate” refers to the compound formed by the interaction of a solvent and a compound. Suitable solvates are pharmaceutically acceptable solvates, such as hydrates, including monohydrates and hemi-hydrates.

The compounds of the invention may also contain naturally occurring or unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). Radiolabeled compounds are useful as therapeutic or prophylactic agents, research reagents, e.g., assay reagents, and diagnostic agents, e.g., in vivo imaging agents. All isotopic variations of the compounds of the invention, whether radioactive or not, are intended to be encompassed within the scope of the invention. For example, if a variable is said or shown to be H, this means that variable may also be deuterium (D) or tritium (T).

“Alkyl” refers to a saturated branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl and propan-2-yl, butyls such as butan-1-yl, butan-2-yl, 2-methyl-propan-1-yl, 2-methyl-propan-2-yl, tert-butyl, and the like. In certain embodiments, an alkyl group comprises 1 to 20 carbon atoms. In some embodiments, alkyl groups include 1 to 10 carbon atoms or 1 to 6 carbon atoms whereas in other embodiments, alkyl groups include 1 to 4 carbon atoms. In still other embodiments, an alkyl group includes 1 or 2 carbon atoms. Branched chain alkyl groups include at least 3 carbon atoms and typically include 3 to 7, or in some embodiments, 3 to 6 carbon atoms. An alkyl group having 1 to 6 carbon atoms may be referred to as a (C₁-C₆)alkyl group and an alkyl group having 1 to 4 carbon atoms may be referred to as a (C₁-C₄)alkyl. This nomenclature may also be used for alkyl groups with differing numbers of carbon atoms. “Alkyl” also includes cycloalkyl.

“Alkenyl” refers to an unsaturated branched or straight-chain hydrocarbon group having at least one carbon-carbon double bond derived by the removal of one hydrogen atom from a single carbon atom of a parent alkene. The group may be in either the Z- or E- form (cis or trans) about the double bond(s). Typical alkenyl groups include, but are not limited to, ethenyl; propenyls such as prop-1-en-1-yl, prop-1-en-2-yl, prop-2-en-1-yl (allyl), and prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, and buta-1,3-dien-2-yl; and the like. In certain embodiments, an alkenyl group has 2 to 20 carbon atoms and in other embodiments, has 2 to 6 carbon atoms. An alkenyl group having 2 to 6 carbon atoms may be referred to as a (C₂-C₆)alkenyl group. “Alkenyl” also includes cycloalkenyl.

“Alkynyl” refers to an unsaturated branched or straight-chain hydrocarbon having at least one carbon-carbon triple bond derived by the removal of one hydrogen atom from a single carbon atom of a parent alkyne. Typical alkynyl groups include, but are not limited to, ethynyl; propynyl; butynyl, 2-pentynyl, 3-pentynyl, 2-hexynyl, 3-hexynyl and the like. In certain embodiments, an alkynyl group has 2 to 20 carbon atoms and in other embodiments, has 2 to 6 carbon atoms. An alkynyl group having 2 to 6 carbon atoms may be referred to as a -(C₂-C₆)alkynyl group.

“Alkoxy” refers to a radical —OR where R represents an alkyl group as defined herein. Representative examples include, but are not limited to, methoxy, ethoxy, propoxy, butoxy, and the like. Typical alkoxy groups include 1 to 10 carbon atoms, 1 to 6 carbon atoms or 1 to 4 carbon atoms in the R group. Alkoxy groups that include 1 to 6 carbon atoms may be designated as —O—(C₁-C₆) alkyl or as —O—(C₁-C₆ alkyl) groups. In some embodiments, an alkoxy group may include 1 to 4 carbon atoms and may be designated as —O—(C₁-C₄) alkyl or as —O—(C₁-C₄ alkyl) groups group.

“Aryl” refers to a monovalent aromatic hydrocarbon group derived by the removal of one hydrogen atom from a single carbon atom of a parent aromatic ring system. Aryl encompasses monocyclic carbocyclic aromatic rings, for example, benzene. Aryl also encompasses bicyclic carbocyclic aromatic ring systems where each of the rings is aromatic, for example, naphthalene. Aryl groups may thus include fused ring systems where each ring is a carbocyclic aromatic ring. In certain embodiments, an aryl group includes 6 to 10 carbon atoms. Such groups may be referred to as C₆-C₁₀ aryl groups. Aryl, however, does not encompass or overlap in any way with heteroaryl as separately defined below. Hence, if one or more carbocyclic aromatic rings is fused with an aromatic ring that includes at least one heteroatom, the resulting ring system is a heteroaryl group, not an aryl group, as defined herein.

“Carbonyl” refers to the radical —C(O) which may also be referred to as —C(═O) group.

“Carboxy” refers to the radical —C(O)OH which may also be referred to as —C(═O)OH.

“Cyano” refers to the radical —CN.

“Cycloalkyl” refers to a saturated cyclic alkyl group derived by the removal of one hydrogen atom from a single carbon atom of a parent cycloalkane. Typical cycloalkyl groups include, but are not limited to, groups derived from cyclopropane, cyclobutane, cyclopentane, cyclohexane, cycloheptane, cyclooctane, and the like. Cycloalkyl groups may be described by the number of carbon atoms in the ring. For example, a cycloalkyl group having 3 to 8 ring members may be referred to as a (C₃-C₈)cycloalkyl, a cycloalkyl group having 3 to 7 ring members may be referred to as a (C₃-C₇)cycloalkyl and a cycloalkyl group having 4 to 7 ring members may be referred to as a (C₄-C₇)cycloalkyl. In certain embodiments, the cycloalkyl group can be a (C₃-C₁₀)cycloalkyl, a (C₃-C₈)cycloalkyl, a (C₃-C₇)cycloalkyl, a (C₃-C₆)cycloalkyl, or a (C₄-C₇)cycloalkyl group and these may be referred to as C₃-C₁₀ cycloalkyl, C3-Cs cycloalkyl, C₃-C₇ cycloalkyl, C₃-C₆ cycloalkyl, or C₄-C₇ cycloalkyl groups using alternative language.

“Heterocyclyl” refers to a cyclic group that includes at least one saturated, partially unsaturated, cyclic ring. Heterocyclyl groups include at least one heteroatom as a ring member. Typical heteroatoms include, O, S and N and are independently chosen. Heterocyclyl groups include monocyclic ring systems and bicyclic ring systems. Bicyclic heterocyclyl groups include at least one non-aromatic ring with at least one heteroatom ring member that may be fused to a cycloalkyl ring or may be fused to an aromatic ring where the aromatic ring may be carbocyclic or may include one or more heteroatoms. The point of attachment of a bicyclic heterocyclyl group may be at the non-aromatic cyclic ring that includes at least one heteroatom or at another ring of the heterocyclyl group. For example, a heterocyclyl group derived by removal of a hydrogen atom from one of the 9 membered heterocyclic compounds shown below may be attached to the rest of the molecule at the 5-membered ring or at the 6-membered ring.

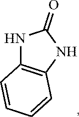

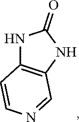

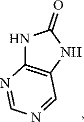

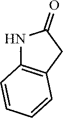

In some embodiments, a heterocyclyl group includes 5 to 10 ring members of which 1, 2, 3 or 4 or 1, 2, or 3 are heteroatoms independently selected from O, S, or N. In other embodiments, a heterocyclyl group includes 3 to 7 ring members of which 1, 2, or 3 heteroatom are independently selected from O, S, or N. In such 3-7 membered heterocyclyl groups, only 1 of the ring atoms is a heteroatom when the ring includes only 3 members and includes 1 or 2 heteroatoms when the ring includes 4 members. In some embodiments, a heterocyclyl group includes 3 or 4 ring members of which 1 is a heteroatom selected from O, S, or N. In other embodiments, a heterocyclyl group includes 5 to 7 ring members of which 1, 2, or 3 are heteroatoms independently selected from O, S, or N. Typical heterocyclyl groups include, but are not limited to, groups derived from epoxides, aziridine, azetidine, imidazolidine, morpholine, piperazine, piperidine, hexahydropyrimidine, 1,4,5,6-tetrahydropyrimidine, pyrazolidine, pyrrolidine, quinuclidine, tetrahydrofuran, tetrahydropyran, benzimidazolone, pyridinone, and the like. Heterocyclyl groups may be fully saturated but may also include one or more double bonds. Examples of such heterocyclyl groups include, but are not limited to, 1,2,3,6-tetrahydropyridinyl, 3,6-dihydro-2H-pyranyl, 3,4-dihydro-2H-pyranyl, 2,5-dihydro-1H-pyrolyl, 2,3-dihydro-1H-pyrolyl, 1H-azirinyl, 1,2-dihydroazetenyl, and the like. Substituted heterocyclyl also includes ring systems substituted with one or more oxo (═O) or oxide (—O^{—}) substituents, such as piperidinyl N-oxide, morpholinyl-N-oxide, 1-oxo-1-thiomorpholinyl, pyridinonyl, benzimidazolonyl, benzo[d]oxazol-2(3H)-onyl, 3,4-dihydroisoquinolin-1(2H)-onyl, indolin-onyl, IH-imidazo[4,5-c]pyridin-2(3H)-onyl, 7H-purin-8(9H)-onyl, imidazolidin-2-onyl, 1H-imidazol-2(3H)-onyl, 1,1-dioxo-1-thiomorpholinyl, and the like. The term “comprising” is meant to be open ended, i.e., all-encompassing and non-limiting. It may be used herein synonymously with “having” or “including”. Comprising is intended to include each and every indicated or recited component or element(s) while not excluding any other components or elements.

“Disease” refers to any disease, disorder, condition, symptom, or indication.

“Halo” or “halogen” refers to a fluoro, chloro, bromo, or iodo group.

“Haloalkyl” refers to an alkyl group in which at least one hydrogen is replaced with a halogen. Thus, the term “haloalkyl” includes monohaloalkyl (alkyl substituted with one halogen atom) and polyhaloalkyl (alkyl substituted with two or more halogen atoms). Representative “haloalkyl” groups include difluoromethyl, 2,2,2-trifluoroethyl, 2,2,2-trichloroethyl, and the like. The term “perhaloalkyl” means, unless otherwise stated, an alkyl group in which each of the hydrogen atoms is replaced with a halogen atom. For example, the term “perhaloalkyl”, includes, but is not limited to, trifluoromethyl, pentachloroethyl, 1,1,1-trifluoro-2-bromo-2-chloroethyl, and the like.

“Heteroaryl” refers to a monovalent heteroaromatic group derived by the removal of one hydrogen atom from a single atom of a parent heteroaromatic ring system. Heteroaryl groups typically include 5- to 14-membered, but more typically include 5- to 10-membered aromatic, monocyclic, bicyclic, and tricyclic rings containing one or more, for example, 1, 2, 3, or 4, or in certain embodiments, 1, 2, or 3, heteroatoms chosen from O, S, or N, with the remaining ring atoms being carbon. In monocyclic heteroaryl groups, the single ring is aromatic and includes at least one heteroatom. In some embodiments, a monocyclic heteroaryl group may include 5 or 6 ring members and may include 1, 2, 3, or 4 heteroatoms, 1, 2, or 3 heteroatoms, 1 or 2 heteroatoms, or 1 heteroatom where the heteroatom(s) are independently selected from O, S, or N. In bicyclic aromatic rings, both rings are aromatic. In bicyclic heteroaryl groups, at least one of the rings must include a heteroatom, but it is not necessary that both rings include a heteroatom although it is permitted for them to do so. For example, the term “heteroaryl” includes a 5- to 7-membered heteroaromatic ring fused to a carbocyclic aromatic ring or fused to another heteroaromatic ring. In tricyclic aromatic rings, all three of the rings are aromatic and at least one of the rings includes at least one heteroatom. For fused, bicyclic and tricyclic heteroaryl ring systems where only one of the rings contains one or more heteroatoms, the point of attachment may be at the ring including at least one heteroatom or at a carbocyclic ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Heteroaryl does not encompass or overlap with aryl as defined above. Examples of heteroaryl groups include, but are not limited to, groups derived from acridine, carbazole, cinnoline, furan, imidazole, indazole, indole, indolizine, isobenzofuran, isochromene, isoindole, isoquinoline, isothiazole, 2H-benzo[d][1,2,3]triazole, isoxazole, naphthyridine, oxadiazole, oxazole, perimidine, phenanthridine, phenanthroline, phenazine, phthalazine, pteridine, purine, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, and the like. In certain embodiments, the heteroaryl group can be between 5 to 20 membered heteroaryl, such as, for example, a 5 to 14 membered or 5 to 10 membered heteroaryl. In certain embodiments, heteroaryl groups can be those derived from thiophene, pyrrole, benzothiophene, 2H-benzo[d] [1,2,3]triazole benzofuran, indole, pyridine, quinoline, imidazole, benzimidazole, oxazole, tetrazole, and pyrazine.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound.

“Pharmaceutically acceptable excipient” refers to a broad range of ingredients that may be combined with a compound or salt of the present invention to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

“Stereoisomer” refers to an isomer that differs in the arrangement of the constituent atoms in space. Stereoisomers that are mirror images of each other and optically active are termed “enantiomers,” and stereoisomers that are not mirror images of one another and are optically active are termed “diastereomers.”

“Subject” includes mammals and humans. The terms “human” and “subject” are used interchangeably herein.

“Therapeutically effective amount” refers to the amount of a compound that, when administered to a subject for treating a disease, or at least one of the clinical symptoms of a disease or disorder, is sufficient to affect such treatment for the disease, disorder, or symptom. As those skilled in the art will recognize. this amount is typically not limited to a single dose but may comprise multiple dosages over a significant period of time as required to bring about a therapeutic or prophylactic response in the subject. Thus, a “therapeutically effective amount” is not limited to the amount in a single capsule or tablet, but may include more than one capsule or tablet, which is the dose prescribed by a qualified physician or medical care provider. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

In some aspects, the compound may be in a form of a salt. Such salts may be anhydrous or associated with water as a hydrate. In some embodiments, the compound may be in a neutral form as a base or an acid.

Also provided are pharmaceutical compositions that include the compound or the pharmaceutically acceptable salt thereof, the tautomer thereof, the pharmaceutically acceptable salt of the tautomer, the stereoisomer of any of the foregoing, or the mixture thereof according to any one of the examples and at least one pharmaceutically acceptable excipient, carrier or diluent. In some such examples, the compound or the pharmaceutically acceptable salt thereof, the tautomer thereof, the pharmaceutically acceptable salt of the tautomer, the stereoisomer of any of the foregoing, or the mixture thereof according to any one of the aspects is present in an amount effective for the treatment of PRMT5-dependent cancers. In some aspects, the pharmaceutical composition is formulated for oral delivery whereas in other embodiments, the pharmaceutical composition is formulated for intravenous delivery. In some embodiments, the pharmaceutical composition is formulated for oral administration once a day or QD, and in some such formulations is a tablet where the effective amount of the active ingredient ranges from 1 mg to 100 mg, from 5 mg to 80 mg, from 10 mg to 50 mg or from 15 to 30 mg.

In some aspects, the subject is a mammal. In some such aspects, the mammal is a rodent. In other aspects, the mammal is a canine. In still other embodiments, the subject is a primate and, in some such embodiments, is a human.

The pharmaceutical compositions or formulations for the administration of the compounds of this invention may conveniently be presented in unit dosage form and may be prepared by any of the methods well known in the art. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The compounds of the invention may be administered via oral, mucosal (including sublingual, buccal, rectal, nasal, or vaginal), parenteral (including subcutaneous, intramuscular, bolus injection, intra-arterial, or intravenous), transdermal, or topical administration. In some aspects, the compounds of the invention are administered via mucosal (including sublingual, buccal, rectal, nasal, or vaginal), parenteral (including subcutaneous, intramuscular, bolus injection, intra-arterial, or intravenous), transdermal, or topical administration. In other aspects, the compounds of the invention are administered via oral administration. In still other embodiments, the compounds of the invention are not administered via oral administration.

The compounds of the invention, the pharmaceutically acceptable salt thereof, the tautomer thereof, the pharmaceutically acceptable salt of the tautomer, the stereoisomer of any of the foregoing, or the mixture thereof may find use in treating a number of conditions.

Compounds and compositions described herein are generally useful for the inhibition of PRMT5. In some aspects, methods of treating PRMT5-mediated disorder in a subject are provided which comprise administering an effective amount of a compound described herein (e.g., a compound of Formula I or a pharmaceutically acceptable salt thereof), to a subject in need of treatment. In certain aspects, the effective amount is a therapeutically effective amount. In certain aspects, the effective amount is a prophylactically effective amount. In certain aspects, the subject is suffering from a PRMT5-mediated disorder (e.g., a cancer, for example a lymphoma, breast cancer, or pancreatic cancer). In other aspects, the subject is susceptible to a PRMT5-mediated disorder (e.g., a cancer, for example a lymphoma, breast cancer, or pancreatic cancer).

As used herein, the term “PRMTS-mediated disorder” means any disease, disorder, or other pathological condition in which PRMT5 is known to play a role. Accordingly, in some aspects, the present disclosure relates to treating or lessening the severity of one or more diseases in which PRMT5 is known to play a role.

In some aspects, herein provided is a method of inhibiting PRMT5 activity in a subject in need thereof comprising administering to the subject an effective amount of a compound described herein (e.g., a compound of Formula I, or a pharmaceutically acceptable salt thereof, or a pharmaceutical composition thereof.

In further aspects, a compound contemplated by the present invention is useful in treating a proliferative disorder, such as cancer. In certain aspects, compounds described herein are useful for treating lymphoma. In some embodiments, the lymphoma is mantle cell lymphoma (MCL). In some embodiments, the lymphoma is acute myeloid lymphoma (AML). In some aspects, the cancer compounds described herein are useful for treating pancreatic cancer. In some aspects, the cancer compounds described herein are useful for treating multiple myeloma (MM). In further aspects, the cancer compounds described herein are useful for treating breast cancer. The breast cancer can be estrogen receptor negative (ER-) or the breast cancer can be progesterone receptor negative (PR-). In further embodiments, the breast cancer can be HER2 negative. In some embodiments, the breast cancer is estrogen receptor negative, progesterone receptor negative and HER2 negative, also referred to herein as “triple negative breast cancer”.

In further aspects, a breast cancer can be a lobular carcinoma in situ (LCIS), a ductal carcinoma in situ (DCIS), an invasive ductal carcinoma (IDC), inflammatory breast cancer, Paget disease of the nipple, Phyllodes tumor, Angiosarcoma, adenoid cystic carcinoma, low-grade adenosquamous carcinoma, medullary carcinoma, mucinous carcinoma, papillary carcinoma, tubular carcinoma, metaplastic carcinoma, micropapary carcinoma, mixed carcinoma, or another breast cancer, including but not limited to triple negative, HER positive, estrogen receptor positive, progesterone receptor positive, HER and estrogen receptor positive, HER and progesterone receptor positive, estrogen and progesterone receptor positive, and HER and estrogen and progesterone receptor positive.

In one aspect, compounds of the invention are useful for treating pancreatic cancer. In another aspect, compounds of the invention are useful for treating NSCLC (non-small cell lung carcinoma. In one embodiment, the NSCLC can be squamous NSCLC. In another embodiment, it can be adenocarcinoma.

In a further aspect, cancer can be GBM. In a further aspect, cancer can be mesothelioma. In one aspect, cancer can be bladder cancer. In another aspect, cancer can be esophageal cancer. In a further aspect, cancer can be melanoma. In one aspect, cancer can be DLBCL, HNSCC or cholangioca.

In some aspects, one or more compounds described herein are useful for treating any PRMT5-mediated or PRMT5-responsive proliferative cell disorder, for example a cancer that is PRMT5 responsive.

In one aspect, a cancer that lacks p53 (e.g., a p53 null cancer) is less sensitive to PRMT5 inhibition than a cancer that is p53 positive. Accordingly, a cancer that is PRMT5 responsive can be a p53 positive cancer. The term “p53 positive” refers to a cancer that does not lack p53 expression and/or activity. In some embodiments, one or more compounds described herein are useful for treating a p53 positive cancer. In some aspects, a greater amount of one or more compounds described herein may be required to treat a p53 negative cancer (e.g. , a p53 null cancer) than a p53 positive cancer.

In some aspects, the disclosure provides a method for identifying subjects having a cancer that is sensitive to treatment with a PRMT5 inhibitor. In some embodiments, the method comprises obtaining a sample from the subject; detecting the presence or absence of p53; and, identifying the subject as having a cancer that is sensitive to treatment with a PRMT5 inhibitor if p53 is present in the sample. Accordingly, in some embodiments, a subject having a p53 positive cancer is identified as a subject for treatment with a PRMT5 inhibitor. In some embodiments, the method further comprises administering to the subject a composition comprising a PRMT5 inhibitor.

In some embodiments, aspects of the disclosure relate to a method for identifying subjects having a cancer that is insensitive (or that has low sensitivity) to treatment with a PRMT5 inhibitor. In some embodiments, the method comprises obtaining a sample from the subject; detecting the presence or absence of p53; and, identifying the subject as having a cancer that is not sensitive (for example, a cancer that is less sensitive than a p53 positive cancer) to treatment with a PRMT5 inhibitor if p53 is absent from the sample (e.g., if the cancer is a p53 null cancer). In some embodiments, a p53 negative cancer (e.g., a p53 null cancer) is treated with a PRMT5 inhibitor, but a greater amount of PRMT5 inhibitor may be required to treat the p53 negative cancer than a p53 positive cancer. However, in some embodiments, a subject having a p53 negative cancer (e.g. , a p53 null cancer) is treated with a therapeutic agent that is not a PRMT5 inhibitor.

By “sample” is meant any biological sample derived from the subject, includes but is not limited to, cells, tissues samples, body fluids (including, but not limited to, mucus, blood, plasma, serum, urine, saliva, and semen), cancer cells, and cancer tissues. Detection of the presence or absence of p53 in the sample may be achieved by any suitable method for detecting p53 nucleic acid or protein, for example, nucleic acid sequencing (e.g., DNA or RNA sequencing), quantitative PCR, Western blotting, etc., or any combination of thereof.

It should be appreciated that in some aspects, one or more of the compounds described herein may be useful for treating other types of cancer, including, but not limited to, acoustic neuroma, adenocarcinoma, adrenal gland cancer, anal cancer, angiosarcoma (e.g., lymphangiosarcoma, lymphangioendotheliosarcoma, hemangio sarcoma), appendix cancer, benign monoclonal gammopathy, biliary cancer (e.g. , cholangiocarcinoma), bladder cancer, brain cancer (e.g., meningioma; glioma, e.g. , astrocytoma, oligodendroglioma; medulloblastoma), bronchus cancer, carcinoid tumor, cervical cancer (e.g. , cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endothelio sarcoma (e.g., Kaposi’s sarcoma, multiple idiopathic hemorrhagic sarcoma), endometrial cancer (e.g., uterine cancer, uterine sarcoma), esophageal cancer (e.g. , adenocarcinoma of the esophagus, Barrett′ s adenocarinoma), Ewing sarcoma, eye cancer (e.g., intraocular melanoma, retinoblastoma), familiar hypereosinophilia, gall bladder cancer, gastric cancer (e.g. , stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g. , B-cell CML, T-cell CML), and chronic lymphocytic leukemia (CLL) (e.g., B-cell CLL, T- cell CLL), follicular lymphoma, chronic lymphocytic leukemia/small lymphocytic lymphoma (CLL/SLL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (e.g., “Waldenstrom’s macro globulinemia”), hairy cell leukemia (HCL), immunoblastic large cell lymphoma, precursor B -lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g. , mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms’ tumor, renal cell carcinoma), liver cancer (e.g. , hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g. , systemic mastocytosis), myelodysplasia syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g. , neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g, cystadenocarcinoma, ovarian embryonal carcinoma, ovarian adenocarcinoma), papillary adenocarcinoma, penile cancer (e.g., Paget’s disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g. , squamous cell carcinoma (SCC), keratoacanthoma (KA), melanoma, basal cell carcinoma (BCC)), small bowel cancer (e.g, appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g. , Paget′ s disease of the vulva).

In some aspects, the method of treating cancer in a subject comprises administering a composition comprising a PRMT5 inhibitor to the subject, wherein treatment with the PRMT5 inhibitor inhibits tumor growth of the cancer by more than about 25%, more than about 50%, more than about 75%, more than about 90% (e.g., 25%-50%, 50%-75%, 75%- 90%, or 90%-100% for example). In some embodiments, the method of treating cancer in a subject comprises administering a composition comprising a PRMT5 inhibitor to the subject, wherein methyl mark of the cancer is reduced more than about 50%, more than about 75%, more than about 80% (e.g., 50%-75%, 50%-80%, 80%-90%, 80%-100%, or 90%-100% for example). A methyl mark refers to protein methylation, for example a histone methylation (e.g., methylation of one or more lysines and/or arginines of a histone protein), or DNA methylation (e.g., epigenetic DNA methylation, for example methylated CpG sites). In some embodiments, the methyl mark level of a cell is a measure of the extent to which histones are methylated in the cell (e.g., at one or more particular lysine and/or arginine positions).

The invention is further described by reference to the following examples, which are intended to exemplify the claimed invention but not to limit it in any way.

### EXAMPLES

Unless otherwise noted, all materials were obtained from commercial suppliers and were used without further purification.

The following Abbreviations are used to refer to various reagents and solvents:

| | |
|---|---|
| AcOH | acetic acid |
| aq or aq. | aqueous |
| Boc | tert-butyloxycarbonyl |
| CLND | chemiluminescent nitrogen detection |
| DAD | diode array detector |
| DCE | 1,2-dichloroethane |
| DCM | dichloromethane |
| DEA | diethylamine |
| DIAD | diisopropyl azodicarboxylate |
| DMA or DMAc | N,N-dimethylacetamide |
| DMF | N,N-dimethylformamide |
| DMSO | dimethyl sulfoxide |
| dppf | 1,1′-bis(diphenylphosphino)ferrocene |
| EDC • HCl | 3-((ethylimino)methyleneamino)-N,N-dimethylpropan-1-amonium chloride |
| ESI or ES | electrospray ionization |
| Et | ethyl |
| Et₂O | diethyl ether |
| EtOH | ethyl alcohol |
| EtOAc | ethyl acetate |
| g | grams |
| h | hour |
| HPLC | high pressure liquid chromatography |
| HATU | 1-[bis(dimethylamino)methylene] -1H-1,2,3 -triazolo [4,5-b]pyridinium 3-oxid hexafluorophosphate |
| HOAt | 1-hydroxy-7-azabenzotriazole |
| iPr | isopropyl |
| iPr₂NEt or DIPEA | N-ethyl diisopropylamine (Hünig’s base) |
| LC MS, LCMS, LC-MS or LC/MS | liquid chromatography mass spectroscopy |
| LG | leaving group (e.g., halogen, mesylate, triflate) |
| LiHMDS | lithium bis(trimethylsilyl)amide |
| m/z | mass divided by charge |
| Me | methyl |
| MeCN/ACN | acetonitrile |
| MeOH | methanol |
| Met | metal species for cross-coupling (e.g., MgX, ZnX, SnR₃, SiR₃, B(OR)₂) |
| mg | milligrams |
| min | minutes |
| mL | milliliters |
| MS | mass spectra |
| MsCl | methanesulfonyl chloride |
| MTBE | tert-butyl methyl ether |
| NMP | 1 -methyl-2-pyrrolidine |
| n-BuLi | n-butyllithium |
| NMR | nuclear magnetic resonance |
| Pd₂(dba)₃ | tris(dibenzylideneacetone)dipalladium(0) |
| Pd(dppf)C1₂•DCM | [1,1′-bis(diphenylphosphino)ferrocene]dichloropalladium(II), complex with DCM |
| Pd(PPh₃)₄ | tetrakis(triphenylphosphine)palladium(0) |
| Ph | phenyl |
| PG or Prot. group | protecting group |
| Prep | preparative |
| PyBroP | bromotripyrrolidinophosphonium hexafluorophosphate |
| RBF | round-bottom flask |
| RP-HPLC | reverse phase high pressure liquid chromatography |
| RT or rt | room temperature |
| R.T. | retention time |
| RuPhos | 2-dicyclohexylphosphino-2′,6′-diisopropoxybiphenyl |
| sat. or sat’d | saturated |
| SFC | supercritical fluid chromatography |
| t-BuOH | tert-butanol |
| TEA or Et₃N | triethylamine |
| TEOS | tetraethyl orthosilicate |
| TFA | trifluoroacetic acid |
| THF | tetrahydrofuran |
| TOF | time of flight |
| UHPLC | ultra-high-performance liquid chromatography |
| Xantphos | 4,5-bis(diphenylphosphino)-9,9-dimethylxanthene |

### General Synthetic Schemes

Method A-1

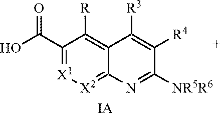

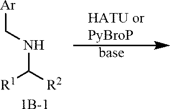

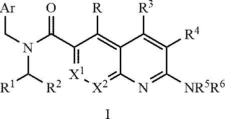

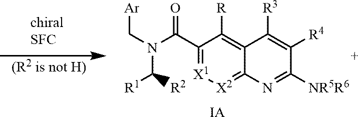

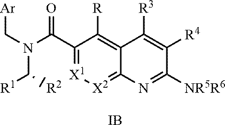

Method A-2

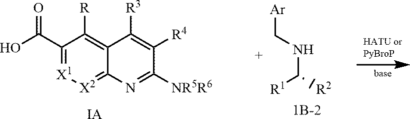

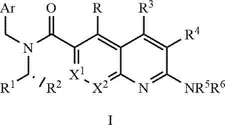

Method B-1

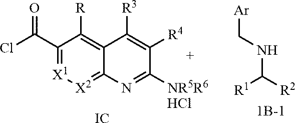

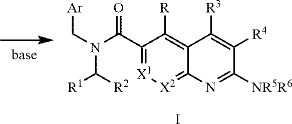

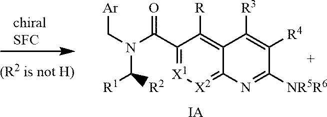

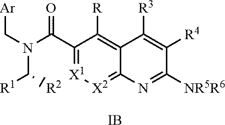

Method B-2

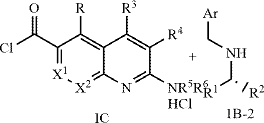

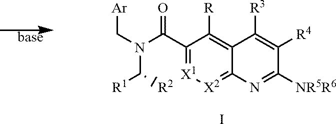

Method C: One pot, two steps synthesis

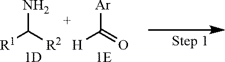

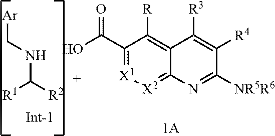

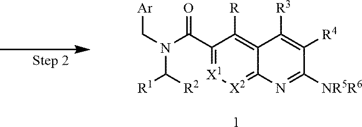

Method D: One step synthesis

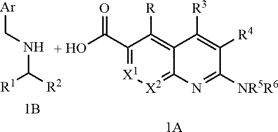

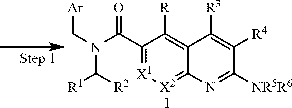

Method A: Compound I can be prepared from the reaction of acid IA and secondary amine IB-1 in the presence of a base such as Et₃N or DIPEA, an activating reagent such as HATU or PyBroP, in a solvent such as DMF or DMAc.

Method B: Compound I can be prepared from the reaction of acid chloride IC and secondary amine IB in the presence of a base such as Et₃N or DIPEA or pyridine, in a solvent such as THF or dioxane or DCM.When R² ≠ H, Method A-PyBroP and Method B are preferred over Method A-HATU. When R² ≠ H, compound I is subjected to chiral SFC to provide the single enantiomers. Alternatively, the chiral amine 1B-2 could be used in the amide coupling reaction (Method A-2 or B-2) to give the final product as a single enantiomer. The more potent (measured by IC₅₀ in HCT116 MTAP null cell viability assay) enantiomer was assigned as the (R)- and the less potent (measured by IC₅₀ in HCT116 MTAP null cell viability assay) and the (S)—.

Method C: Compound I can be prepared by a variety of small scale one pot, two step protocols as illustrated in General Scheme Method C. Primary amine 1D can be combined with aldehyde 1E in the method specified solvents and after imine formation and reduction will yield a secondary amine (Int-1) as a crude product. The secondary amine (Int-1) was reacted with acid IA with the method specific coupling reagents to yield product I after HPLC purification.

Method D: Compounds were synthesized by a variety of small scale one step protocols as illustrated in general scheme D. Secondary amine (IB) was combined with acid (IA) and treated with the method specific coupling reagents and solvent mixture to yield product I after HPLC purification.

### Analytical U/HPLC

The following equipment was used for analytical UHPLC: Waters Acquity system equipped with an Acquity BEH C18 (1.7_{µ}m, 2.1 x 50 mm) with a linear gradient of a binary solvent system using a flow rate of 0.5 mL/min and DAD at ambient temperature, combined with MS detection SQD I. Linear gradients used (H₂O/CH₃CN/HCO₂H (95/5/0.1% to 0/100/0.1%)). Agilent Infinity I/II -TOF6230B /CLND Antek 8060 equipped with Acquity BEH C18 (1.7 µm, 2.1 × 50 mm) with a linear gradient of a binary solvent system using a flow rate of 0.75 mL/min combined with DAD. Linear gradients used (H₂O/MeOH/HCO₂H (95/5/0.1% to 0/100/0.1%)).

Preparative HPLC The following equipment was used for Prep-HPLC: Shimadzu Nexera X2 equipped with a Merck Chromolith SpeedROD RP-18E (5 µm, 10 × 100 mm) with a linear gradient of a binary solvent system using a flow rate between 4 and 7 mL/min and UV detection at 254 nm, combined with MS detecting on a Shimadzu LCMS-2020. Linear gradients used (H₂O/MeOH/HCO₂H (95/5/0.1% to 0/100/0.1%))

### Intermediates

Intermediate 1: 2-amino-3-methylquinoline-6-carboxylic acid.

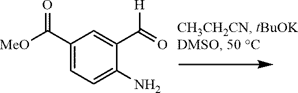

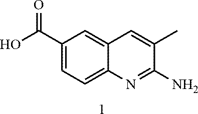

To a stirred solution of propionitrile (59.9 mL, 837 mmol, Loba Chem) in DMSO (750 mL) in a round-bottomed flask was added potassium tert-butoxide (1.0 M in THF, 837 mL, 837 mmol) at 0° C. under N₂ atmosphere. Then the mixture was stirred for 15 min before it was treated with methyl 4-amino-3-formylbenzoate (75 g, 419 mmol, Abovchem) in small portions then heated at 50 °Cfor 16 h. The reaction mixture was cooled to RT, diluted with water (1000 mL) and extracted with ethyl acetate (2 x 1000 mL). The aqueous layer pH was adjusted near 6.5 by using 1.5 N HC1 solution. The resulting solid was filtered, washed with water (2 × 1000 mL), followed by acetone (2 × 1000 mL), and dried under vacuum overnight. The solid was stirred in MTBE (1000 mL) for 12 h, filtered, and dried under vacuum to provide acid 1 (52.3 g, 259 mmol, 61.8% yield). m/z (ESI): 203.0 (M+H)⁺. ¹H NMR (400 MHz, DMSO-d₆): 8 ppm 10.01 (br s, 1 H), 8.24 (d, J = 2.0 Hz, 1 H), 7.91 (dd, J = 8.7, 2.0 Hz, 1 H), 7.86 (s, 1 H), 7.46 (d, J = 8.7 Hz, 1 H), 6.67 (s, 2 H), 2.22 (s, 3 H).

Intermediate 1a: 2-amino-3-methyl-5,6,7,8-tetrahydroquinoline-6-carboxylic acid

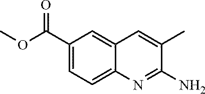 [SMILES: COC(=O)c1ccc2nc(N)c(C)cc2c1]

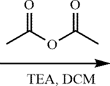

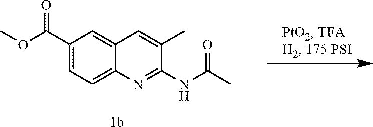

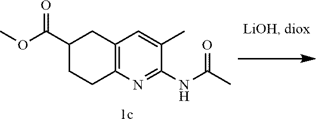

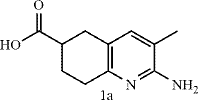

Methyl 2-acetamido-3-methylquinoline-6-carboxylate (1b). To a 150-mL round-bottomed flask was added methyl 2-amino-3-methylquinoline-6-carboxylate (1.00 g, 4.62 mmol) and triethylamine (1.3 mL, 9.25 mmol, Sigma-Aldrich Corporation) in DCM (23 mL). Then acetic anhydride (0.5 mL, 5.78 mmol, Sigma-Aldrich Corporation) was added dropwise to the reaction mixture. The resulting reaction mixture was stirred at RT overnight, while under an inert (N₂) atmosphere. The reaction mixture was concentrated in vacuo. The residue was triturated from EtOAc and heptane. The precipitate was collected and the solids were washed with heptane, to afford the title compound (1b, 1.02 g, 3.96 mmol, 86% yield) as a white solid. ¹H NMR (400 MHz, DMSO-d₆) δ ppm 10.31 (br s, 1 H), 8.57 (br s, 1 H), 8.37 (br s, 1 H), 8.13 (br d, J=8.2 Hz, 1 H), 7.92 (br d, J=8.4 Hz, 1 H), 3.92 (br s, 3 H), 2.32 - 2.38 (m, 3 H), 2.18 (br s, 3 H). m/z (ESI): 259.1 (M+H)⁺.

Methyl 2-acetamido-3-methyl-5,6,7,8-tetrahydroquinoline-6-carboxylate (1c). A glass reaction vessel was charged with methyl 2-acetamido-3-methylquinoline-6-carboxylate (0.35 g, 1.36 mmol) and platinum(IV) oxide (0.062 g, 0.271 mmol, Sigma-Aldrich) in TFA (3.3 mL). The reaction chamber was closed and sealed. Then the vessel was purged with argon (3x) before purging with H₂ (3x). Then H₂ (175 psi) was added and the vial was stirred at 6° C. for 16 h. The reaction mixture was concentrated in vacuo and the residue diluted with DCM:CHC1₃:MeOH (1:1:0.1), then the mixture was treated with sat. aq. NaHCO₃, to adjust to pH~10. The layers were separated and the aqueous layer was extracted with DCM (3x). The combined organic extracts were filtered through a pad of celite, then the celite was rinsed with 1:1 DCM:MeOH (3x). The collected filtrate was dried over MgSO₄, filtered, and concentrated in vacuo to afford the title compound (1c, 0.21 g, 0.80 mmol, 59% yield) as a light-yellow solid. ¹H NMR (400 MHz, DMSO-d₆) δ ppm 9.85 (br s, 1 H), 7.36 (s, 1 H), 3.63 - 3.66 (m, 3 H), 2.74 -2.96 (m, 5 H), 2.09 - 2.18 (m, 1 H), 2.07 (s, 3 H), 2.00 (s, 3 H), 1.78 - 1.92 (m, 1 H). m/z (ESI): 263.1 (M+H)⁺. Note: This reaction was performed with a Biotage® Endeavor™ catalyst screening system

2-Amino-3-methyl-5,6,7,8-tetrahydroquinoline-6-carboxylic acid (1a). To a 150-mL round-bottomed flask was added methyl 2-acetamido-3-methyl-5,6,7,8-tetrahydroquinoline-6-carboxylate (0.630 g, 2.402 mmol) and 1,4-dioxane (4.8 mL). Then lithium hydroxide (1.0 M in H₂O) (12 mL, 12.01 mmol, Sigma-Aldrich) was added to the reaction mixture. The reaction mixture was heated to 60° C. and stirred for 3 h. The reaction mixture was partially concentrated (to remove 1,4-dioxane) in vacuo. The residue was treated with 1 N HC1 to adjust to pH~7-8. The precipitate was collected by filtration and the solids were washed with water. The solids were dried in a reduced-pressure oven (40° C.) for 3 h. This afforded 2-amino-3-methyl-5,6,7,8-tetrahydroquinoline-6-carboxylic acid (1a, 0.25 g, 1.21 mmol, 51% yield) as tan solid. ¹H NMR (400 MHz, DMSO-d₆) δ ppm 6.84 (br s, 1 H), 5.27 (br s, 2 H), 2.53 - 2.59 (m, 2 H), 2.49 - 2.53 (m, 1 H), 2.27 - 2.36 (m, 1 H), 1.90 (br s, 4 H), 1.73 (br s, 2 H), 1.59 (br s, 1 H). m/z (ESI): 207.2 (M+H)⁺.

Intermediate 2: 2-amino-3-(methyl-D₃)quinoline-6-carboxylic acid. This acid was prepared from propionitrile-3,3,3-D₃ (C/D/N Isotopes Inc.) following the procedures described for acid 1. m/z (ESI): 206.1 (M+H)⁺. ¹H NMR (METHANOL-D₄, 400 MHz) δ 8.52 (d, 1H, J=1.9 Hz), 8.3-8.4 (m, 2H), 7.72 (d, 1H, J=8.8 Hz).

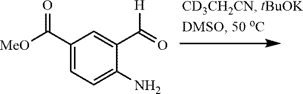

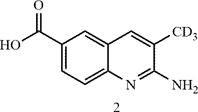

Intermediate 3: 2-amino-3-chloroquinoline-6-carboxylic acid.

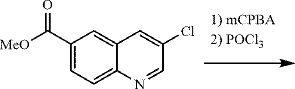

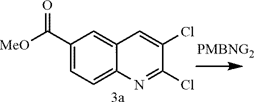

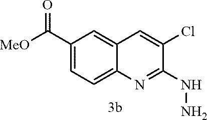

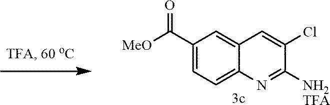

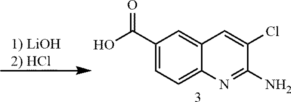

Methyl 2,3-dichloroquinoline-6-carboxylate (3a). A solution of methyl 3-chloroquinoline-6-carboxylate (25.0 g, 113 mmol, PharmaBlock) in DCM (500 mL) at 0° C. was added mCPBA (64.9 g, 226 mmol) then stirred at RT for 16 h. The reaction was quenched with saturated sodium bicarbonate (1500 mL) and extracted with DCM (2 × 1000 mL). The organic extract was washed with brine (1000 mL), dried over Na₂SO₄, filtered, and concentrated. The residue was stirred with diethyl ether (2 × 500 mL) for 10 min and diethyl ether was decanted. The obtained solid was dried under reduced pressure to afford 3-chloro-6-(methoxycarbonyl)quinoline 1-oxide (23 g, 97 mmol, 86% yield) as a brown solid. ¹H NMR (400 MHz, DMSO-d₆): δ ppm 8.95 (d, J = 1.7 Hz, 1 H), 8.76 (d, J = 1.9 Hz, 1 H), 8.58 (d, J = 9.1 Hz, 1 H), 8.39 (t, J = 1.3 Hz, 1 H), 8.26 (dd, J = 9.1, 1.9 Hz, 1 H), 3.96 (s, 3 H).

A mixture of 3-chloro-6-(methoxycarbonyl)quinoline 1-oxide (8.0 g, 33.7 mmol) and POC1₃ (30 mL) was stirred at 80 C for 2 h then concentrated under reduced pressure. The residue was treated with saturated sodium bicarbonate (500 mL) and extracted with ethyl acetate (2 × 1000 mL). The organic extract was washed with brine NaCl (500 mL), dried over Na₂SO₄, filtered and concentrated. The crude material was absorbed onto a plug of silica gel and purified by chromatography (7% EtOAc in hexanes) to give methyl 2,3-dichloroquinoline-6-carboxylate (3a, 6.5 g, 25.6 mmol, 76% yield) as a brown solid. m/z (ESI): 256.2 (M+H)⁺. ¹H NMR (400 MHz, Chloroform-d): δ ppm 8.53 (d, J = 1.9 Hz, 1 H), 8.29 -8.40 (m, 2 H), 8.07 (dt, J = 8.8, 0.7 Hz, 1 H), 4.02 (s, 3 H).

Methyl 3-chloro-2-((4-methoxybenzyl)amino)quinoline-6-carboxylate (3b). A mixture of methyl 2,3-dichloroquinoline-6-carboxylate (3a, 9.0 g, 35.1 mmol) and 4-methoxybenzylamine (9.18 mL, 70.3 mmol) in N-methyl-2-pyrrolidinone (100 mL) was stirred at 120° C. for 6 h. The reaction mixture was cooled to RT, diluted with water (500 mL), and extracted with EtOAc (2 × 400 mL). The organic extract was washed with brine (500 mL) and dried over Na₂SO₄. The mixture was filtered and concentrated. The crude material was absorbed onto a plug of silica gel and purified by chromatography (15-17% EtOAc in hexanes) to provide compound 3b (11.9 g, 33.3 mmol, 95% yield) as a white solid. m/z (ESI): 357.1 (M+H)⁺. ¹H NMR (400 MH_{z}, DMSO-d₆): δ ppm 8.39 (dd, J= 9.4, 1.3 Hz, 2 H), 8.00 (dd, J = 8.8, 2.1 Hz, 1 H), 7.91 (t, J= 6.1 Hz, 1 H), 7.58 (d, J= 8.8 Hz, 1 H), 7.30 - 7.40 (m, 2 H), 6.83 -6.92 (m, 2 H), 4.66 (d, J = 6.0 Hz, 2 H), 3.87 (s, 3 H), 3.81 (s, 3 H).

Methyl 2-amino-3-chloroquinoline-6-carboxylate 2,2,2-trifluoroacetate (3c). At RT, methyl 3-chloro-2-((4-methoxybenzyl)amino)quinoline-6-carboxylate (3b, 12.0 g, 28.9 mmol) was treated with TFA (22.28 mL, 289 mmol). The mixture was stirred at 60° C. for 16 h, then concentrated under reduced pressure. The residue was triturated with diethyl ether and diethyl ether was decanted. The white solid obtained was dried under high vacuum to afford compound 3c (5.45 g, 23.1 mmol, 80%). m/z (ESI): 237.1 (M+H)⁺.

2-Amino-3-chloroquinoline-6-carboxylic acid (3). To a stirred solution of methyl 2-amino-3-chloroquinoline-6-carboxylate 2,2,2-trifluoroacetate (3c, 18.0 g, 51.3 mmol) in MeOH and THF (4:1, 250 mL) was added a solution of lithium hydroxide (6.15 g, 257 mmol) in water (50 mL). After 16 h, the reaction mixture was concentrated under reduced pressure. The residue was cooled with an ice bath and acidified to pH 6.5 by the addition of 1.5 N HC1. The resulting off-white solid was filtered and dried under vacuum to afford acid 3 (13.1 g, 49.4 mmol, 96% yield). m/z (ESI): 223.1 (M+H)⁺. ¹H NMR (400 MHz, DMSO-d₆) δ ppm 8.33 - 8.39 (m, 2 H), 7.99 (dd, J=8.8, 2.1 Hz, 1 H), 7.52 (d, J=8.8 Hz, 1 H), 7.06 - 7.18 (m, 2 H), 6.73 (br s, 1 H).

Intermediate 4: 2-amino-3-bromoquinoline-6-carboxylic acid.

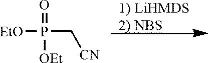

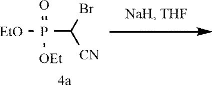

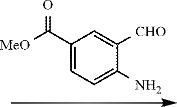 [SMILES: COC(=O)c1ccc(N)c(C=O)c1]

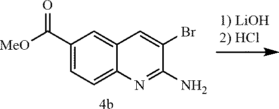

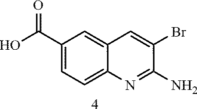

Diethyl (bromo(cyano)methyl)phosphonate (4a). To a stirred solution of diethyl (cyanomethyl)phosphonate (100.0 g, 565 mmol) in THF (3 L) was added a solution of lithium bis(trimethylsilyl)amide (1.0 M in THF, 621 mL, 621 mmol) at 0° C. and it was stirred at same temperature for 30 min. The reaction mixture was cooled to -78° C. and 1-bromopyrrolidine-2,5-dione (121.0 g, 677 mmol) was added. The reaction mixture was allowed to warm slowly to -10° C. and stirred at -10° C. to 0° C. for 3 h. The reaction mixture was quenched with 1.5 N HC1 solution (2 L) and extracted with DCM (2 x 2 L). The combined organic layers were dried over anhydrous Na₂SO₄, filtered and concentrated under reduced pressure. The crude material was absorbed onto a plug of silica gel and purified by chromatography through a Redi-Sep pre-packed silica gel column (80 g), eluting with 20 % to 25% ethyl acetate in hexanes to give diethyl (bromo(cyano)methyl)phosphonate (4a, 100.0 g, 392 mmol, 69% yield) as a brown oil. m/z (ESI): 256.0 (M+H)⁺. ¹H NMR (400 MHz, DMSO-d₆): δ ppm 5.92 (d, J= 16.3 Hz, 1 H), 4.30 - 4.21 (m, 4 H), 1.33-1.31 (m, 6 H).

Methyl 2-amino-3-bromoquinoline-6-carboxylate (4b). To a suspension of sodium hydride (60% in mineral oil, 1.674 g, 41.9 mmol) in THF (75 mL) under N₂ at 0° C. was added a solution of compound 4a (10.0 g, 39.1 mmol) in THF (75 mL). The resulting mixture was stirred for 30 min then treated with a solution of methyl 4-amino-3-fortnylbenzoate (5.0 g, 27.9 mmol, Abovchem) in THF (15 mL). The reaction mixture was allowed to stir at RT for 4 h then quenched with water (100 mL). The resulting light-yellow solid was filtered and dried under vacuum to provide 4b (4.7 g, 16.7 mmol, 60% yield). m/z (ESI): 280.9 (M+H)⁺. ¹H NMR (400 MH_{z}, DMSO-d₆): δ 8.59 (s, 1 H), 8.39 (d, J = 2.0 Hz, 1 H), 8.02 (dd, J= 8.8, 2.1 Hz, 1 H), 7.54 (d, J= 8.8 Hz, 1 H), 7.11 (s, 2 H), 3.88 (s, 3 H).

2-Amino-3-bromoquinoline-6-carboxylic acid (4). To a stirred solution of compound 4b (4.7 g, 16.0 mmol) in THF (30 mL), water (15 mL), and MeOH (15 mL) was added lithium hydroxide (2.68 g, 64.0 mmol). After stirring for 4 h, the reaction mixture was concentrated, and the residue was acidified with 1.5 N HC1 solution to a pH of ~6.0 to 6.5. The off-white solid was filtered and dried under high vacuum to give acid 4 (4.5 g, 100% yield). m/z (ESI): 266.9 (M+H)⁺. ¹H NMR (400 MHz, DMSO-d₆): δ 12.87 (s, 1 H), 8.57 (s, 1 H), 8.35 (d, J = 2.0 Hz, 1 H), 8.01 (dd, J = 8.8, 2.0 Hz, 1 H), 7.50 (d, J = 2.0 Hz, 1 H), 7.05 (s, 2 H).

Intermediate 5: 2-amino-7-fluoro-3-methylquinoline-6-carboxylic acid.

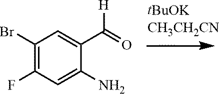

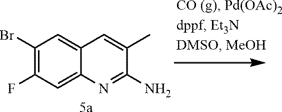

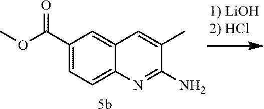

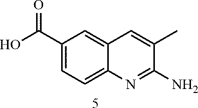

6-Bromo-7-fluoro-3-methylquinolin-2-amine (5a). To a round-bottomed flask was added propionitrile (19.69 mL, 275 mmol, Loba Chemie), potassium tert-butoxide (1 M in THF, 275 mL, 275 mmol, Spectrochem) and DMSO (300 mL). The mixture was cooled with an ice bath and treated with 2-amino-5-bromo-4-fluorobenzaldehyde (30 g, 138 mmol, Biogene) portion wise. The mixture was heated at 50° C. for 12 h, then cooled to RT and quenched with water (1 L) slowly. The precipitated white solid was filtered, washed with water (500 mL), and dried under vacuum. The white solid was triturated with diethyl ether (500 mL) and filtered to obtain compound 5a (25 g, 98 mmol, 71% yield). m/z (ESI): 255.0 (M+H)⁺. ¹H NMR (400 MHz, DMSO-d₆): δ ppm 7.97 (d, J = 8.0 Hz, 1 H), 7.73 (s, 1 H), 7.29 (d, J = 10.9 Hz, 1 H), 6.60 (s, 2 H), 2.19 (s, 3H).

Methyl 2-amino-7-fluoro-3-methylquinoline-6-carboxylate (5b). To a 250 mL autoclave pressure reactor, compound 5a (10 g, 39.2 mmol), dppf (6.52 g, 11.76 mmol), triethylamine (13.6 mL, 98 mmol) and palladium (II) acetate (1.76 g, 7.84 mmol) in MeOH (60 mL) and dimethyl sulfoxide (60 mL) were added. The reaction mixture was allowed to stir at 80° C. under CO atmosphere (100 psi) for 36 h. The reaction mixture was cooled to RT, diluted with water (100 mL) and extracted with EtOAc (3 × 100 mL). The organic layer was concentrated under reduced pressure, and crude material was absorbed onto a plug of silica gel and purified by chromatography, eluting with a gradient of 70-100% EtOAc in hexanes, to provide compound 5b (6.0 g, 25.6 mmol, 65% yield) as off-white solid. m/z (ESI): 235.1 (M+H)⁺. ¹H NMR (400 MHz, DMSO-d₆): δ ppm 8.24 (d, J = 8.4 Hz, 1 H), 7.87 (s, 1 H), 7.17 (dd, J = 13.4, 0.7 Hz, 1 H), 6.90 (s, 2 H), 3.86 (s, 3 H), 2.19 (s, 3H).

2-Amino-7-fluoro-3-methylquinoline-6-carboxylic acid (5). To a stirred solution of compound 5b (6.0 g, 25.6 mmol) in THF (60 mL), MeOH (30 mL) and water (30 mL) was added lithium hydroxide monohydrate (3.22 g, 77 mmol). After stirring for 12 h at RT, the mixture was concentrated, and the residue was acidified with 1.5 N HC1 to pH = 6. The precipitated brown solid was filtered, washed with MeOH (50 mL) and twice azeotroped with toluene (30 mL) to provide acid 5 (5 g, 19.04 mmol, 89% yield). m/z (ESI): 221.1 (M+H)⁺. ¹H NMR (400 MH_{z}, DMSO-d₆): δ ppm 8.35 (d, J= 8.0 Hz, 1 H), 8.13 (s, 1 H), 7.99 (br s, 2 H), 7.42 (d, J = 12.1 Hz, 1 H), 2.22 (s, 3 H).

Intermediate 6: 2-amino-7-chloro-3-methylquinoline-6-carboxylic acid.

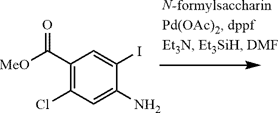

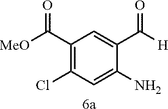

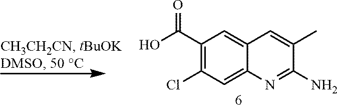

Methyl 4-amino-2-chloro-5-formylbenzoate (6a). Methyl 4-amino-2-chloro-5-iodobenzoate (398 mg, 1.28 mmol, Combi-Blocks Inc.), N-formylsaccharin (809 mg, 3.83 mmol, Combi-Blocks Inc.), palladium(ii) acetate (28.7 mg, 0.128 mmol, Umicore AG & Co.KG.), 1,4-bis(diphenylphosphino)butane (109 mg, 0.256 mmol, Sigma-Aldrich Corporation), and triethylsilane (193 mg, 0.265 mL, 1.661 mmol, Sigma-Aldrich Corporation) were mixed in N, N-dimethylformamide (3 mL) in a sealed vial under a nitrogen atmosphere. The mixture was stirred at RT for 5 min. The stirring was stopped, and triethylamine (388 mg, 0.539 mL, 3.83 mmol) was added carefully to avoid as much mixing as possible (bubbling observed). The reaction mixture was heated at 115° C. for 1 h then cooled to RT and partitioned between EtOAc (80 mL) and saturated NH₄C1 (60 mL). The organic layer was separated, washed with brine (50 mL), dried over MgSO₄, filtered, and concentrated in vacuo. Chromatographic purification of the residue (silica gel, 0 -30% EtOAc in heptane) gave 6a (122 mg, 0.571 mmol, 45% yield) as a yellow solid. MS (ESI, +ve) m/z: 213.9 [M + H]⁺. ¹H NMR (400 MHz, CHLOROFORM-d) δ ppm 9.86 (1 H, s) 8.21 (1 H, s) 6.73 (1 H, s) 3.90 (3 H, s).

2-Amino-7-chloro-3-methylquinoline-6-carboxylic acid (6). This acid was prepared from 6a following the procedures described for acid 1. MS (ESI, +ve) m/z: 236.9 [M + H]⁺. ¹H NMR (400 MHz, DMSO-d6) δ ppm 8.32 (1 H, s) 8.19 (1 H, s) 7.71 (1 H, s) 2.28 (3 H, s).

Intermediates 7 and 8: 2-amino-4-methylquinoline-6-carboxylic acid and 2-amino-4-chloroquinoline-6-carboxylic acid. These acids were purchased from Labnetwork.

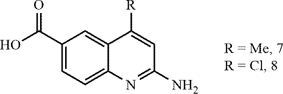

Intermediate 9: 2-((4-methoxybenzyl)amino)-3,4-dimethylquinoline-6-carboxylic acid.

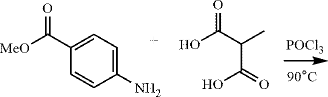

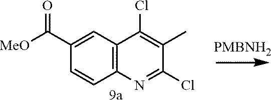

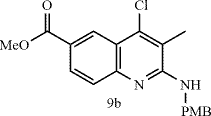

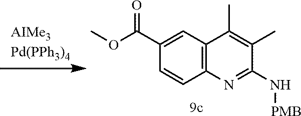

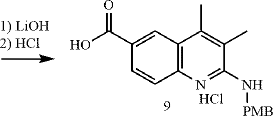

Methyl 2,4-dichloro-3-methylquinoline-6-carboxylate (9a): A mixture of 4-amino methyl benzoate (5.0 g, 33.1 mmol) and methylmalonic acid (4.3 g, 36.4 mmol) in phosphorous oxychloride (15.41 mL, 165 mmol) was heated at 90° C. for 5 h. It was cooled to RT, and slowly transferred (using a pipette) to an ice-cold water solution of saturated KOAc (100 mL). When all the reaction was quenched, the pH of the water solution was adjusted to 10 by the addition of solid NaOH (exothermic). The mixture was extracted with DCM (4 × 70 mL). The organics were dried over MgSO₄, filtered and concentrated in vacuo to give a yellow residue. The residue was purified by silica gel chromatography (0-100% EtOAc in heptane) to provide methyl 2,4-dichloro-3-methylquinoline-6-carboxylate (9a, 5.45 g, 20.18 mmol, 61% yield) as a white solid. m/z (ESI): 270.0 (M+H)⁺.

Methyl 4-chloro-2-((4-methoxybenzyl)amino)-3-methylquinoline-6-carboxylate (9b): A mixture of methyl 2,4-dichloro-3-methylquinoline-6-carboxylate (9a, 3.0 g, 11.1 mmol) and 4-methoxybenzylamine (2.93 mL, 22.21 mmol) in NMP (37 mL) was heated at 100° C. for 16 h. The reaction mixture was cooled to RT then diluted with saturated NaHCO₃ (50 mL) and extracted with EtOAc (3 × 50 mL). The organics were dried over MgSO₄, filtered and concentrated in vacuo to give the crude material as yellow oil (contains NMP). The crude material was purified by silica gel chromatography (0-100% EtOAc in heptane) to provide methyl 4-chloro-2-((4-methoxybenzyl)amino)-3-methylquinoline-6-carboxylate (9b, 3.6 g, 9.71 mmol, 87% yield) as a white solid. m/z (ESI): 371.0 (M+H)⁺.

Methyl 2-((4-methoxybenzyl)amino)-3,4-dimethylquinoline-6-carboxylate (9c): To a 20 mL microwave vial was added methyl 4-chloro-2-((4-methoxybenzyl)amino)-3-methylquinoline-6-carboxylate (9b, 0.80 g, 2.1 mmol) and Pd(PPh₃)₄ (0.25 g, 0.22 mmol) in 1,4-dioxane (13 mL). To the solution was then added trimethylaluminum (2.1 mL of 2.0 M solution in toluene, 4.2 mmol). The vial was sealed then heated at 130° C. in a microwave reactor for 3 h. The reaction mixture was carefully poured into saturated aqueous NaHCO₃ (50 mL). The resulting solid was filtered and washed with EtOAc. The filtrate was extracted with EtOAc (3 × 30 mL). The organics were concentrated. The residue was purified by silica gel chromatography (0-100% EtOAc in heptane) to provide methyl 2-((4-methoxybenzyl)amino)-3,4-dimethylquinoline-6-carboxylate (9c, 396 mg, 1.13 mmol, 52% yield) as a pale-yellow solid. m/z (ESI): 351.2 (M+H)⁺.

2-((4-Methoxybenzyl)amino)-3,4-dimethylquinoline-6-carboxylic acid hydrochloride (9): A mixture of methyl 2-((4-methoxybenzyl)amino)-3,4-dimethylquinoline-6-carboxylate (9c, 150 mg, 0.43 mmol) in water/MeOH/THF (1:1:1, 8.56 mL) was treated with lithium hydroxide monohydrate (90 mg, 2.14 mmol) then heated at 60° C. for 2 h. The solvents were concentrated in vacuo. The residue was adjusted to pH 2-3 by the addition of HC1 (2 N) then evaporated (with the aid of toluene) to give 2-((4-methoxybenzyl)amino)-3,4-dimethylquinoline-6-carboxylic acid hydrochloride (9, contaminated with 5 equiv. of LiC1) as an off-white solid. The product was used in the next step with no further purification. Theoretical yield was considered. m/z (ESI): 337.1 (M+H)⁺.

Intermediate 10: 7-amino-6-methyl-1,8-naphthyridine-3-carboxylic acid hydrochloride.

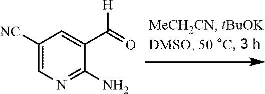

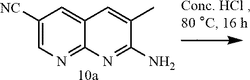

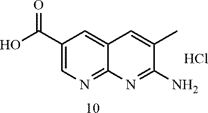

7-Amino-6-methyl-1,8-naphthyridine-3-carbonitrile (10a). This molecule was prepared in a manner similar to that described for 5a. m/z (ESI): 185.1 (M+H)⁺. ¹H NMR (400 MHz, DMSO-d₆): δ ppm 8.91 (d, J = 2.4 Hz, 1 H), 8.55 (d, J = 2.4 Hz, 1 H), 7.82 (d, J = 1.3 Hz, 1 H), 7.42 (s, 2 H), 2.24 (s, 3 H).

7-Amino-6-methyl-1,8-naphthyridine-3-carboxylic acid hydrochloride (10). A mixture of compound 10a (6.6 g, 35.8 mmol) and conc. hydrochloric acid (66 mL) was heated to 80° C. for 16 h then cooled to RT and diluted with water (60 mL). The resulting solid was filtered and dried under vacuum to obtain acid 10 (2.5 g, 12.2 mmol, 34% yield). m/z (ESI): 204.1 (M+H)⁺. ¹H NMR (400 MHz, DMSO-d₆): δ ppm 13.72 (s, 1 H), 9.12 (d, J = 2.2 Hz, 1 H), 8.88 (d, J = 2.2 Hz, 1 H), 8.75 (s, 2 H), 8.31 (s, 1 H), 2.32 (s, 3 H).

Intermediate 11: 7-amino-6-chloro-1,8-naphthyridine-3-carboxylic acid.

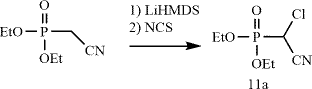

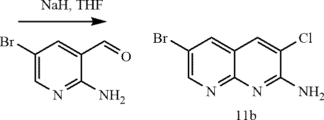

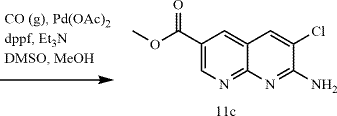

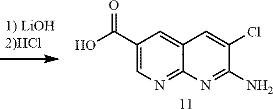

Diethyl (chloro(cyano)methyl)phosphonate (11a). This molecule was prepared in a manner similar to that described for 4a. m/z (ESI): 212.1 (M-H)+. ¹H NMR (400 MHz, DMSO-d₆): δ ppm 6.20 (d, J = 17.3 Hz, 1 H), 4.29 - 4.22 (m, 4 H), 1.33-1.30 (m, 6 H).

6-Bromo-3-chloro-1,8-naphthyridin-2-amine (11b). To a 500 mL 3-neck RB flask, sodium hydride (2.98 g, 74.6 mmol) was added in THF (300 mL). A solution of compound 11a (15.79 g, 74.6 mmol) in THF (75 mL) was added at 0° C. and stirred for 30 min at same temperature. The mixture was treated with 2-amino-5-bromonicotinaldehyde (10 g, 49.7 mmol) then stirred at RT for 4 h. The mixture was diluted with water (400 mL). The resulting yellow solid was filtered and azeotroped with toluene to give compound 11b (7.0 g, 27.1 mmol, 54% yield). m/z (ESI): 258.0 (M+H)⁺. ¹H NMR (400 MHz, DMSO): δ ppm 8.77 (d, J = 2.6 Hz, 1 H), 8.38 (d, J = 2.6 Hz, 1 H), 8.24 (s, 1 H), 7.36 (s, 2 H).

7-Amino-6-chloro-1,8-naphthyridine-3-carboxylic acid (11). This molecule was prepared from 11b following the procedures (carbonylation followed by saponification) described for acid 5. m/z (ESI): 224.0 (M+H)⁺. ¹H NMR (400 MHz, DMSO d₆): δ ppm 13.19 (s, 1 H), 9.16 (d, J=2.4 Hz, 1 H), 8.66 (d, J=2.4 Hz, 1 H), 8.44 (s, 1 H), 7.60 (s, 2 H).

Intermediate 12: 7-amino-6-bromo-1,8-naphthyridine-3-carboxylic acid.

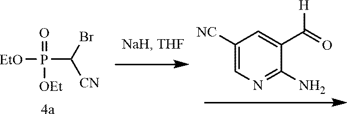

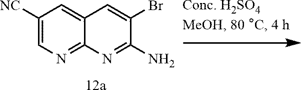

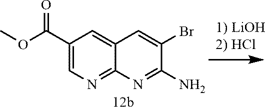

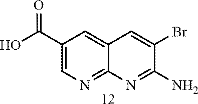

7-Amino-6-bromo-1,8-naphthyridine-3-carbonitrile (12a). To a suspension of sodium hydride (60% in mineral oil, 1.63 g, 68 mmol) in THF (150 mL) at 0° C. was added a solution of diethyl (bromo(cyano)methyl)phosphonate (17.4 g, 68 mmol) in THF (100 mL). The resulting mixture was stirred for 30 min under N₂ atmosphere. To the reaction mixture was added 6-amino-5-formylnicotinonitrile (5.0 g, 34 mmol) in small portions. The mixture was stirred at room temperature for 4 h then quenched with water (50 mL). The brown solid was filtered and dried under vacuum to provide 7-amino-6-bromo-1,8-naphthyridine-3-carbonitrile (12a, 5.5 g, 22.08 mmol, 65% yield). m/z (ESI): 249.1 (M+H)⁺. ¹H NMR (400 MHz, DMSO-d₆): δ ppm 9.03 (d, J= 2.4 Hz, 1 H), 8.63 (d, J= 2.5 Hz, 1 H), 8.50 (s, 1 H).

Methyl 7-amino-6-bromo-1,8-naphthyridine-3-carboxylate (12b). A stirred solution of 7-amino-6-bromo-1,8-naphthyridine-3-carbonitrile (12a, 5.0 g, 20.07 mmol) in MeOH (60 mL) at room temperature was treated with sulfuric acid (60 mL) and stirred at 80° C. for 4 h. The reaction mixture was concentrated under vacuum and diluted with water (160 mL). The product was precipitated by adding a solution of aq. NaOH (pH of ~7.5-8.5). The solid was filtered, washed with MTBE and dried under vacuum to give methyl 7-amino-6-bromo-1,8-naphthyridine-3-carboxylate (12c, 4.6 g, 16.3 mmol, 81% yield) as an off-white solid. ¹H NMR (400 MHz, DMSO-d₆): δ ppm 9.18 (d, J = 2.5 Hz, 1 H), 8.70 (d, J = 2.5 Hz, 1 H), 8.64 (s, 1 H), 3.90 (s, 3 H). m/z (ESI): 282.0 (M+H)⁺.

7-Amino-6-bromo-1,8-naphthyridine-3-carboxylic acid (12). To a stirred solution of 7-amino-6-bromo-1,8-naphthyridine-3-carboxylate (12b, 4.7 g, 16.7 mmol) in THF (50 mL), MeOH (50 mL), and water (50 mL) was added lithium hydroxide monohydrate (2.09 g, 50.0 mmol) at room temperature and stirred for 12 h. The reaction mixture was concentrated under reduced pressure. The crude residue was diluted with water (50 mL) and acidified with 1 N HC1 solution to pH 6-6.5. The precipitated solid was filtered and dried under vacuum to give 7-amino-6-bromo-1,8-naphthyridine-3-carboxylic acid (12, 3.1 g, 11.5 mmol, 69% yield) as a light-yellow solid. ¹H NMR (400 MHz, DMSO-d6): δ ppm 9.17 (d, J = 2.5 Hz, 1 H), 8.65 (d, J = 2.4 Hz, 1 H), 8.62 (s, 1 H), 7.43 (br s, 2 H). m/z (ESI): 267.9 (M+H)⁺

Intermediate 13: 2-amino-3-methyl-1,7-naphthyridine-6-carboxylic acid.

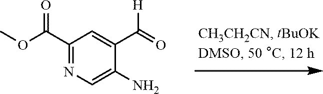

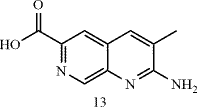

This molecule was prepared following the procedures described for acid 1. m/z (ESI): 204.1 (M+H)⁺. ¹H NMR (400 MHz, TFA d): δ ppm 9.74 (s, 1 H), 9.17 (s, 1 H), 8.61 (s, 1 H), 2.81 (s, 3 H).

Intermediate 14: 2-amino-3-chloro-1,7-naphthyridine-6-carboxylic acid.

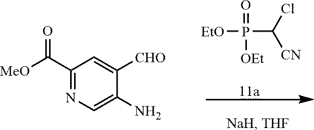

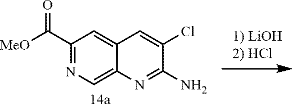

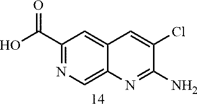

This molecule was prepared following the procedures described for acid 4. m/z (ESI): 224.0 (M+H)⁺. ¹H NMR (400 MHz, DMSO-d₆): δ 11.01 (s, 1H), 8.68 (s, 1H), 8.41 (s, 1H), 8.22 (s, 1H), 7.23 (s, 2H).

Intermediate 15: 2-amino-3-bromo-1,7-naphthyridine-6-carboxylic acid.

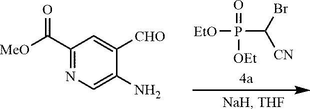

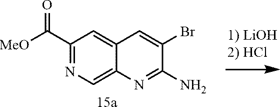

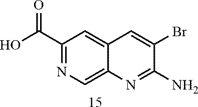

This molecule was prepared following the procedures described for acid 4. m/z (ESI): 268.0 (M+H)⁺. ¹H NMR (400 MHz, DMSO-d₆): δ ppm 8.71 (s, 1 H), 8.61 (s, 1 H), 8.24 (s, 1 H), 7.18 (s, 2 H).

Intermediate 16: 2-amino-3-methylquinoline-6-carbonyl chloride hydrochloride.

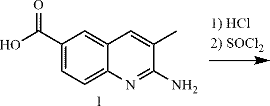

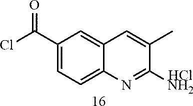

To a suspension of 2-amino-3-methylquinoline-6-carboxylic acid (1, 10.29 g, 50.9 mmol) in DCM (200 mL) under nitrogen was added HC1 (14 mL of 4.0 M in dioxane, 56.0 mmol, Sigma-Aldrich). The mixture was stirred for 10 mins then evaporated to dryness under reduced pressure. The resulting yellow solid was treated with thionyl chloride (91 g, 55.7 mL, 763 mmol, Sigma-Aldrich) followed by DMF (25 µL) then heated at 55° C. for 1 h. The reaction mixture was cooled to RT, diluted with DCM (100 mL), and filtered via a fritted funnel. The solid was rinsed with 1:1 DCM : heptane (10 mL) and briefly dried under a stream of nitrogen to give Intermediate 16 (12.86 g, 98% yield) as a free-flowing yellow powder. This material was used without further manipulation.

TABLE 1

Acid chlorides below were prepared in a manner similar to that described for Intermediate 16

| Int. # | Chemical Structure | Name | m/z (ESI: (M+H)⁺ |
|---|---|---|---|
| 17 | 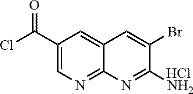 [SMILES: Nc1nc2ncc(C(=O)Cl)cc2cc1Br ; Cl] | 7-amino-6-bromo-1,8-naphthyridine-3-carbonyl chloride hydrochloride | |
| 21 | 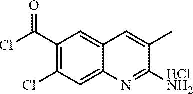 [SMILES: Cc1cc2cc(C(=O)Cl)c(Cl)cc2nc1N ; Cl] | 2-amino-7-chloro-3-methylquinoline-6-carbonyl chloride hydrochloride | 251.0(M+MeOH) |

Intermediate 18: 6-bromo-N,N-bis-Boc-3-methyl-1,8-naphthyridin-2-amine.

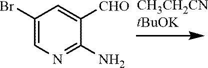

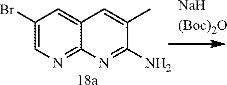

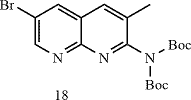

6-Bromo-3-methyl-1,8-naphthyridin-2-amine (18a): To a 25-mL glass vial was added 2-amino-5-bromo-3-formylpyridine (1.79 g, 8.93 mmol, Combi-Blocks Inc.), propiononitrile (0.96 mL, 13.39 mmol, Aldrich), and potassium t-butoxide (150 g, 13.39 mmol). The mixture was heated at 70° C. for 2 h then cooled to RT and added in water. The precipitated orange solid was filtered and collected. It was suspended in EtOAc, sonicated for several minutes, filtered and dried in an oven to afford 6-bromo-3-methyl-1,8-naphthyridin-2-amine (18a, 1.19 g, 5.00 mmol, 56% yield) as a light tan solid. m/z (ESI): 238/240 (M+H)⁺. ¹H NMR (400 MHz, DMSO-d₆) δ ppm 8.66 (d, J=2.51 Hz, 1 H), 8.27 (d, J=2.72 Hz, 1 H), 7.75 (d, J=0.84 Hz, 1 H), 6.86 (br s, 2 H), 2.23 (d, J=1.05 Hz, 3 H).

6-Bromo-N,N-bis-Boc-3-methyl-1,8-naphthyridin-2-amine (18): A mixture of 6-bromo-3-methyl-1,8-naphthyridin-2-amine (18a, 890 mg, 3.74 mmol) in 15 mL of THF at RT was treated with sodium hydride (299 mg of 60% wt. in mineral oil, 7.48 mmol). The reaction mixture was stirred at RT for 10 min then treated with di-tert-butyl dicarbonate (1.71 g, 7.85 mmol) in THF (2 mL). The mixture was stirred at RT for 90 min then heated at 60° C. for 18 h. The mixture was cooled to RT, diluted with saturated aq. NH₄C1 and extracted with EtOAc. The organic extract was washed with water, dried over Na₂SO₄, filtered and concentrated. The residue was purified by chromatography through a Redi-Sep pre-packed silica gel column (40 g), eluting with a gradient of 0-3% 2 M NH₃•MeOH in DCM, to provide Intermediate 18 (0.61 g, 50% yield) as a tan solid. m/z (ESI): 438/440 (M+H)⁺. ¹H NMR (400 MHz, DMSO-d₆) δ ppm 9.11 (d, J=2.51 Hz, 1 H), 8.81 (d, J=2.51 Hz, 1 H), 8.39 (d, J=0.84 Hz, 1 H), 2.37 (d, J=0.63 Hz, 3 H), 1.36 (s, 18 H).

Intermediate 19: 2-amino-3-ethylquinoline-6-carboxylic acid.

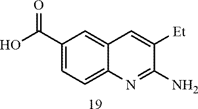

This acid was made in a manner similar to that described for Intermediate 1. m/z (ESI): 217.0 (M+H)⁺.

Intermediate 20: 2-(bis(4-methoxybenzyl)amino)-3-methylquinoline-6-carboxylic acid.

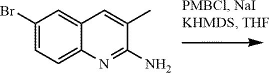

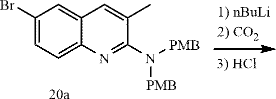

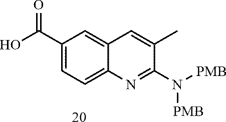

A 500 mL round bottom flask was charged with 6-bromo-3-methylquinolin-2-amine (25 g, 105 mmol, PharmaBlock), 1-(chloromethyl)-4-methoxybenzene (49.5 g, 316 mmol), sodium iodide (7.90 g, 52.7 mmol) and THF (250 mL) and this reaction mixture was stirred at 0° C. for 30 min. To the reaction mixture at 0° C. was added KHMDS (316 mL of 1 M in THF, 316 mmol) dropwise. The reaction mixture was stirred at RT for 16 h then quenched with ice cold water (250 mL) and extracted with ethyl acetate (3 × 100 mL). The organic layer was washed with brine solution (2 × 50 mL), dried (Na₂SO₄), filtered and concentrated under reduced pressure. The crude residue was purified by column chromatography over silica gel (1% EtOAc in petroleum ether) to give 6-bromo-N,N-bis(4-methoxybenzyl)-3-methylquinolin-2-amine (20a, 25 g, 52.4 mmol, 50% yield) as a white solid. m/z (ESI): 477/479 (M+H)⁺).

THF (50 mL) was cooled to -78° C. then treated with a solution of n-butyllithium (2.5 M in hexanes, 25.1 mL, 62.8 mmol). The resulting mixture was stirred for 10 min at -78° C. then treated with 6-bromo-N,N-bis(4-methoxybenzyl)-3-methylquinolin-2-amine (20a, 25 g, 52.4 mmol) in THF (10 mL) slowly. The mixture was stirred for 10 min at -78° C. then treated with solid crushed CO₂. The reaction mixture stirred at RT for 30 min then quenched with ice water and extracted with ethyl acetate (2 × 20 mL). The aqueous layer was acidified with 1.5 N HC1 solution to adjust the pH to 6. The precipitated tan solid was filtered and dried to give 2-(bis(4-methoxybenzyl)amino)-3-methylquinoline-6-carboxylic acid (20, 24 g). m/z (ESI): 443.2 (M+H)⁺).

Intermediate 22: 7-amino-6-bromo-5-methyl-1,8-naphthyridine-3-carboxylic acid.

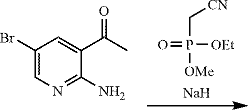

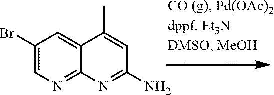

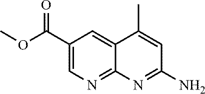 [SMILES: COC(=O)c1cnc2nc(N)cc(C)c2c1]

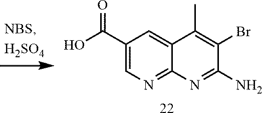

To a 100 mL RBF was added sodium hydride (1.40 g, 34.9 mmol) in tetrahydrofuran (80 mL), then the mixture was cooled to 0° C. Then, diethyl (cyanomethyl)phosphonate (4.94 g, 27.9 mmol; GLR Innovations Chemicals) was added to the mixture and stirred for 1 h, while the temperature was maintained at 0° C. A solution of 1-(2-amino-5-bromopyridin-3-yl)ethan-1-one (5 g, 23.25 mmol) in THF was added to the mixture at 0° C. and the overall mixture was stirred at RT for 16 h. The reaction mixture was diluted with saturated aqueous NH₄C1 (100 mL) and the mixture was filtered through a pad of Celite. The filtrate was collected, then the aqueous layer was extracted with EtOAc (2 × 200 mL).The organic extract was washed with saturated aqueous NaCl (100 mL) and dried over Na₂SO₄. The solution was filtered and concentrated in vacuo to give 6-bromo-4-methyl-1,8-naphthyridin-2-amine (3.0 g, 12.60 mmol, 54.2 % yield) as a brown solid. m/z (ESI): 240.1 (M+H)⁺.

To a 100-mL tinyclave was added 6-bromo-4-methyl-1,8-naphthyridin-2-amine (2 g, 8.40 mmol), PdOAc₂ (0.38 g, 1.68 mmol), dppf (1.40 g, 2.52 mmol) and triethylamine (2.9 mL, 21.00 mmol) in dimethyl sulfoxide (12 mL) and methanol (12 mL). To this CO (gas) was purged under 10 kg pressure. The reaction mixture was stirred at 100° C. for 24 hrs. The reaction mixture was diluted with water (100 mL) and extracted with CH2C1₂ ( 2 × 200 mL). The organic extract was dried over Na₂SO₄. The solution was filtered and concentrated in vacuo to give the crude material as a tan solid. The crude material was absorbed onto a plug of silica gel and purified by chromatography through a SiO₂ Column, eluting with a gradient of 60 % to 100% EtOAc in hexane, to provide methyl 7-amino-5-methyl-1,8-naphthyridine-3-carboxylate (0.7 g, 3.22 mmol, 38% yield) as brown solid. ¹H NMR(400 MHz, DMSO): δ ppm 8.52 (d, J=2.3 Hz, 1 H), 7.73 (d, J=2.2 Hz, 1 H), 6.93 (s, 2 H), 5.82 (q, J=1.1 Hz, 1 H), 3.78 (s, 3 H), 2.29 (d, J=1.1 Hz, 3 H). m/z (ESI): 218.3 (M+H)⁺.

To a 50 mL RBF was added methyl 7-amino-5-methyl-1,8-naphthyridine-3-carboxylate (1 g, 4.60 mmol) in sulfuric acid (0.25 mL, 4.69 mmol). Then NBS (1.64 g, 9.21 mmol) was added to the mixture, then the mixture was heated and stirred at 90° C. for 3 h. The reaction mixture was diluted with water and quenched with solid NaOH to adjust the pH to ~ 10, then the mixture was stirred for 1 h. The reaction mixture was acidified with conc. HC1 solution to adjust the pH to ~6-6.5 to generate precipitate. The solids were collected by filtration and dried to afford methyl 7-amino-6-bromo-5-methyl-1,8-naphthyridine-3-carboxylate (1.3 g, 4.39 mmol, 95% yield) as an off-white solid. m/z (ESI): 282.0/284.0 (M+H)⁺.

Intermediate 23: 2-amino-3-iodoquinoline-6-carboxylic acid-lithium chloride

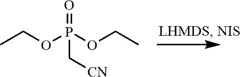

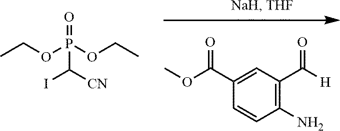

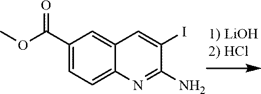

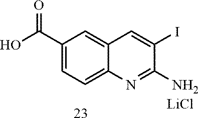

To a solution of diethyl (cyanomethyl)phosphonate (1.09 g, 6.15 mmol, Combi-Blocks Inc.) in 25 mL of THF at 0° C. was added LHMDS (6.15 mL of 1 M in THF solution, 6.15 mmol, Aldrich). The reaction mixture was stirred at 0° C. for 30 min, then cooled to -78° C. and treated with NIS (1.38 g, 6.15 mmol, Aldrich) as a solid. The reaction mixture was warmed to -10° C. and stirred at -10° C. to -5° C. for 2 h. It was quenched with saturated NH₄C1 (10 mL) and extracted with EtOAc (2 × 50 mL). The organic extract was washed with 5 mL of brine, dried over Na₂SO₄, filtered, and concentrated in vacuo. The crude material was purified by chromatography through a Redi-Sep pre-packed silica gel column (40 g), eluting with 20% to 75% EtOAc in heptane, to provide diethyl (cyanoiodomethyl)phosphonate (0.80 g, 2.64 mmol, 42.9% yield) as a brown oil. m/z (ESI): 304 (M+H)⁺.

Sodium hydride (110 mg of 60% wt in mineral oil, 2.76 mmol) was added to a solution of diethyl (cyanoiodomethyl)phosphonate (781 mg, 2.58 mmol) in 10 mL of THF at 0° C. The resulting yellow mixture was stirred at 0° C. for 30 min then treated with a solution of methyl 4-amino-3-formylbenzoate (330 mg, 1.842 mmol) in 5 mL of THF. The mixture was stirred at RT for 30 min then treated with sodium hydride (20 mg of 60% wt in mineral oil) and stirred at RT for 3 h. The reaction mixture was quenched with saturated aqueous NH₄C1 (5 mL) and extracted with 2 × 25 mL of EtOAc. The organic layers were combined and washed with 5 mL of water, and concentrated. Purification of the residue on a silica gel column (10% to 75% EtOAc in DCM) gave a brown sticky solid (32 mg). The material was stirred in 2 mL of heptane and the liquid was decanted. The resulting brown solid was dried to give methyl 2-amino-3-iodoquinoline-6-carboxylate (24 mg, 0.073 mmol, 4% yield). m/z (ESI): 328.5 (M+H)⁺. ¹H NMR (400 MHz, DMSO-d₆) δ 8.80 (s, 1H), 8.35 (d, J = 2.0 Hz, 1H), 8.02 (d, J=9.41 Hz, 1H), 7.52 (d, J=8.99 Hz, 1H), 6.87-7.14 (m, 2H), 3.88 (s, 3H).

Methyl 2-amino-3-iodoquinoline-6-carboxylate (24 mg, 0.073 mmol) in 1 mL of THF and 1 mL of MeOH and 0.5 mL of water was treated with LiOH-H₂O (6 mg, 0.14 mmol) and heated at 45° C. for 1 h. The mixture was treated with 0.08 mL of 2 N HC1 and concentrated to dryness. The residue was coevaportaed with 2 × 2 mL of toluene to give 2-amino-3-iodoquinoline-6-carboxylic acid-lithium chloride (23) (30 mg) as a brown solid which was used as crude material. m/z (ESI): 315 (M+H)⁺.

Intermediate 24: 7-amino-6-iodo-1,8-naphthyridine-3-carboxylic acid hydrochloride

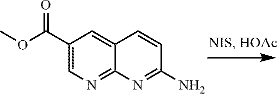

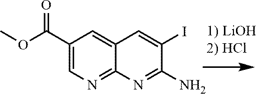

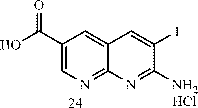

To a mixture of methyl 7-amino-1,8-naphthyridine-3-carboxylate (1.20 g, 5.91 mmol) in acetic acid (35 mL) at RT was added NIS (4.65 g, 20.67 mmol, Aldrich). The mixture was stirred at RT for 30 min then heated at 120° C. for 4 h. Acetic acid was evaporated under high vacuum. The residue was basified with saturated aqueous sodium carbonate. The insoluble solid was filtered, washed with 3 × 10 mL of water followed by 3 × 10 mL of EtOAc. The black solid was dried to give methyl 7-amino-6-iodo-1,8-naphthyridine-3-carboxylate (1.30 g, 3.95 mmol, 67% yield). The filtrate was extracted with 50 mL of EtOAc/EtOH = (4/1) and the organic solution was concentrated and purified on a silica gel column (1% to 10% MeOH in EtOAc) to give methyl 7-amino-6-iodo-1,8-naphthyridine-3-carboxylate (200 mg). m/z (ESI): 330 (M+H)⁺.

Lithium hydroxide hydrate (0.365 g, 8.69 mmol) was added to a mixture of methyl 7-amino-6-iodo-1,8-naphthyridine-3-carboxylate (1.30 g, 3.95 mmol) in 3 mL of THF, 3 mL of MeOH and 3 mL of water. The mixture was heated at 45° C. for 1 h then concentrated. The residue was treated with 4 N HCl and stirred at RT for 10 min. The brown solid was filtered, washed with 2 × 5 mL of water followed by 2 × 3 mL of ether, collected and dried to give 7-amino-6-iodo-1,8-naphthyridine-3-carboxylic acid hydrochloride (1.51 g, 4.30 mmol, 109% yield) which was used as crude material. m/z (ESI): 315.8 (M+H)⁺. ¹H NMR (DMSO-d₆, 400 MHz) δ 10.1 (br s, 1H), 9.14 (d, 1H, J=2.1 Hz), 8.91 (s, 1H, J=2.1 Hz), 8.76 (s, 1H), 7.78 (br s, 1H), 6.81 (br s, 1H).

Intermediate 25: 2-amino-3,5-dimethylquinoline-6-carboxylic acid.

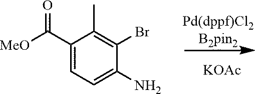

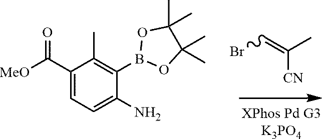

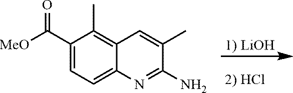

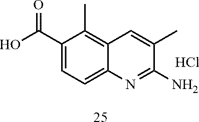

To a 150-mL round-bottomed flask was added methyl 4-amino-3-bromo-2-methylbenzoate (1.74 g, 7.13 mmol) and bis(pinacolato)diboron (3.62 g, 14.26 mmol) in 1,4-dioxane (23.76 mL). To the solution was then added potassium acetate (2.099 g, 21.39 mmol). The mixture was degassed bubbling Ar for 5 min and [1,1′-bis(diphenylphosphino)ferrocene]dichloropalladium(ii), complex with dichloromethane (0.582 g, 0.713 mmol) was added. The reaction was then left stirring at 100° C. After 5 h the LCMS showed formation of the desired product along with dehalogenated compound. Water (30 mL) was added to the mixture which was extracted with DCM (3×25 mL), dried over MgSO₄, filtered and concentrated to give a black oil/solid. The crude material was absorbed onto a plug of silica gel and purified by chromatography through a Redi-Sep pre-packed silica gel column (120 g), eluting with a gradient of 0% to 30% (3:1 EtOAc:EtOH) in DCM (desired product elutes at 5%), to provide methyl 4-amino-2-methyl-3-(4,4,5,5-tetramethyl-1,3,2-dioxaborolan-2-yl)benzoate (830 mg, 2.85 mmol, 40.0% yield) as light-yellow solid. m/z (ESI): 292.3 (M+H)⁺.

To a red-capped vial was added methyl 4-amino-2-methyl-3-(4,4,5,5-tetramethyl-1,3,2-dioxaborolan-2-yl)benzoate (700 mg, 2.404 mmol), 3-bromo-2-methylacrylonitrile (mix of isomers) (351 mg, 2.404 mmol), potassium phosphate tribasic (1021 mg, 4.81 mmol) and methanesulfonato(2-dicyclohexylphosphino-2′,4′,6′-tri-i-propyll-1,1′-biphenyl)(2′-amino-1,1′-biphenyl-2-yl)palladium(ii) (204 mg, 0.240 mmol) in 1,4-dioxane (6011 µL) and water (6011 µL). The solution was heated to 90° C. and left stirring for 2 hours. Water (15 mL) was added and the solid formed was filtered and washed with DCM to give methyl 2-amino-3,5-dimethylquinoline-6-carboxylate (160 mg, 0.695 mmol, 28.9% yield) as light-yellow solid. m/z (ESI): 231.1 (M+H)⁺.

To a 150 mL round-bottomed flask was added methyl 2-amino-3,5-dimethylquinoline-6-carboxylate (0.092 g, 0.4 mmol) in water (2.67 mL), methanol (2.67 mL) and tetrahydrofuran (2.67 mL). Lithium hydroxide, monohydrate (0.084 g, 2.000 mmol) was then added and the mixture was left stirring at 70° C. for 4 hours. The solvents were concentrated in vacuo, and the resulting water layer was adjusted to pH 3 by the addition of HCl 2 N. The water was then evaporated (with the aid of toluene) to give 2-amino-3,5-dimethylquinoline-6-carboxylic acid hydrochloride (25) (contaminated with 5 eq LiCl) as off-white solid. The product was used in the next step with no further purification. m/z (ESI): 217.1 (M+H)⁺.

Intermediate 26: 2-methyl-N-((5-(trifluoromethyl)pyridin-2-yl)methyl)propan-1-amine.

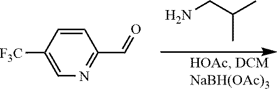

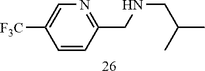

To a mixture of 5-(trifluoromethyl)picolinaldehyde (3.02 g, 17.27 mmol, AstaTech Inc) and isobutylamine (1.48 g, 20.21 mmol, Combi-Blocks Inc.) in DCM (50 mL) at RT was added acetic acid (1.11 g, 18.48 mmol). The mixture was stirred at RT for 30 min then treated with sodium triacetoxyborohydride (5.49 g, 25.9 mmol, Aldrich). The mixture was stirred at RT for 1 h then neutralized with saturated aqueous Na₂CO₃. The layers were separated and the aqueous layer was extracted with DCM. The combined organic phase was dried over Na₂SO₄ and concentrated in vacuo. The crude material was purified by silica gel chromatography (0-100% EtOAc/EtOH (3/1) in heptane) to afford the title compound (26, 2.81 g, 70% yield) as a brown oil. m/z (ESI): 233.0 (M+H)⁺. ¹H NMR (400 MHz, CHLOROFORM-d) δ ppm 8.81 (s, 1 H), 7.88 (dd, J=8.1, 2.1 Hz, 1 H), 7.50 (d, J=8.1 Hz, 1 H), 3.98 (s, 2 H), 2.46 (d, J=6.6 Hz, 2 H), 1.73 - 1.84 (m, 2 H), 0.94 (d, J=6.6 Hz, 6 H). ¹⁹ F NMR (376 MHz, CHLOROFORM-d) δ ppm -62.26 (s, 3 F).

TABLE 2

Secondary amines 27 to 64 were prepared in a manner similar to that described for Intermediate 26.

| Int. # | Chemical Structure | Name | m/z (ESI: (M+H)⁺ |
|---|---|---|---|
| 27 | 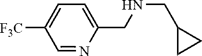 [SMILES: FC(F)(F)c1ccc(CNCC2CC2)nc1] | 1-cyclopropyl-N-((5-(trifluoromethyl)pyridin-2-yl)methyl)methanamine | 231.0 |
| 28 | 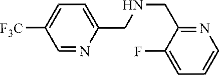 [SMILES: Fc1cccnc1CNCc1ccc(C(F)(F)F)cn1] | 1-(3-fluoropyridin-2-yl)-N-((5-(trifluoromethyl)pyridin-2-yl)methyl)methanamine | 286.0 |
| 29 | 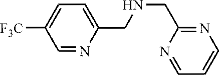 [SMILES: FC(F)(F)c1ccc(CNCc2ncccn2)nc1] | 1-(pyrimidin-2-yl)-N-((5-(trifluoromethyl)pyridin-2-yl)methyl)methanamine | 269.0 |
| 30 | 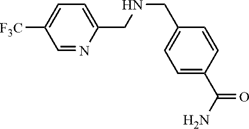 [SMILES: NC(=O)c1ccc(CNCc2ccc(C(F)(F)F)cn2)cc1] | 4-((((5-(trifluoromethyl)pyridin-2-yl)methyl)amino)methyl)benzamide | 310.0 |
| 31 | 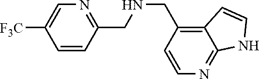 [SMILES: FC(F)(F)c1ccc(CNCc2ccnc3[nH]ccc23)nc1] | N-((1H-pyrrolo[2,3-b]pyridin-4-yl)methyl)-1-(5-(trifluoromethyl)pyridin-2-yl)methanamine | 307.1 |
| 32 | 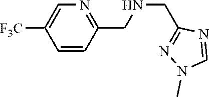 [SMILES: Cn1cnc(CNCc2ccc(C(F)(F)F)cn2)n1] | 1-(1-methyl-1H-1,2,4-triazol-3-yl)-N-((5-(trifluoromethyl)pyridin-2-yl)methyl)methanamine | 271.9 |
| 33 | 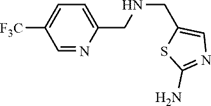 [SMILES: Nc1ncc(CNCc2ccc(C(F)(F)F)cn2)s1] | 5-((((5-(trifluoromethyl)pyridin-2-yl)methyl)amino)methyl)thiazol-2-amine | 289.0 |
| 34 | 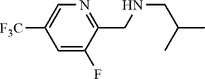 [SMILES: CC(C)CNCc1ncc(C(F)(F)F)cc1F] | N-((3-fluoro-5-(trifluoromethyl)pyridin-2-yl)methyl)-2-methylpropan-1-amine | 250.9 |
| 35 | 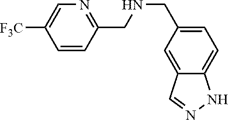 [SMILES: FC(F)(F)c1ccc(CNCc2ccc3[nH]ncc3c2)nc1] | N-((1H-indazol-5-yl)methyl)-1-(5-(trifluoromethyl)pyridin-2-yl)methanamine | 307.1 |
| 36 | 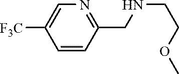 [SMILES: COCCNCc1ccc(C(F)(F)F)cn1] | 2-methoxy-N-((5-(trifluoromethyl)pyridin-2-yl)methyl)ethan-1-amine | 234.9 |
| 37 | 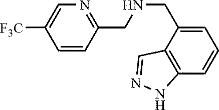 [SMILES: FC(F)(F)c1ccc(CNCc2cccc3[nH]ncc23)nc1] | N-((1H-indazol-4-yl)methyl)-1-(5-(trifluoromethyl)pyridin-2-yl)methanamine | 307.1 |
| 38 | 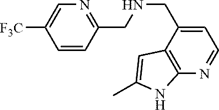 [SMILES: Cc1cc2c(CNCc3ccc(C(F)(F)F)cn3)ccnc2[nH]1] | 1-(2-methyl-1H-pyrrolo[2,3-b]pyridin-4-yl)-N-((5-(trifluoromethyl)pyridin-2-yl)methyl)methanamine | 321.0 |
| 39 | 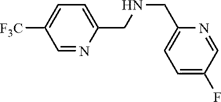 [SMILES: Fc1ccc(CNCc2ccc(C(F)(F)F)cn2)nc1] | 1-(5-fluoropyridin-2-yl)-N-((5-(trifluoromethyl)pyridin-2-yl)methyl)methanamine | 285.9 |
| 40 | 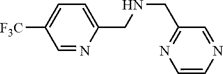 [SMILES: FC(F)(F)c1ccc(CNCc2cnccn2)nc1] | 1-(pyrazin-2-yl)-N-((5-(trifluoromethyl)pyridin-2-yl)methyl)methanamine | 268.9 |
| 41 | 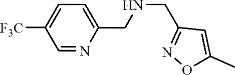 [SMILES: Cc1cc(CNCc2ccc(C(F)(F)F)cn2)no1] | 1-(5-methylisoxazol-3-yl)-N-((5-(trifluoromethyl)pyridin-2-yl)methyl)methanamine | 271.9 |
| 42 | 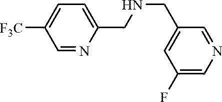 [SMILES: Fc1cncc(CNCc2ccc(C(F)(F)F)cn2)c1] | 1-(5-fluoropyridin-3-yl)-N-((5-(trifluoromethyl)pyridin-2-yl)methyl)methanamine | 286.1 |
| 43 | 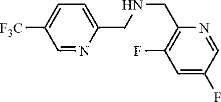 [SMILES: Fc1cnc(CNCc2ccc(C(F)(F)F)cn2)c(F)c1] | 1-(3,5-difluoropyridin-2-yl)-N-((5-(trifluoromethyl)pyridin-2-yl)methyl)methanamine | 303.9 |
| 44 | 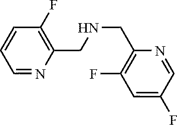 [SMILES: Fc1cnc(CNCc2ncccc2F)c(F)c1] | 1-(3,5-difluoropyridin-2-yl)-N-((3-fluoropyridin-2-yl)methyl)methanamine | 253.9 |
| 45 | 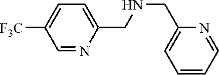 [SMILES: FC(F)(F)c1ccc(CNCc2ccccn2)nc1] | 1-(pyridin-2-yl)-N-((5-(trifluoromethyl)pyridin-2-yl)methyl)methanamine | 268.0 |
| 46 | 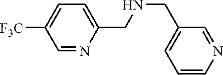 [SMILES: FC(F)(F)c1ccc(CNCc2cccnc2)nc1] | 1-(pyridin-3-yl)-N-((5-(trifluoromethyl)pyridin-2-yl)methyl)methanamine | 268.0 |
| 47 | 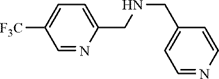 [SMILES: FC(F)(F)c1ccc(CNCc2ccncc2)nc1] | 1-(pyridin-4-yl)-N-((5-(trifluoromethyl)pyridin-2-yl)methyl)methanamine | 268.0 |
| 48 | 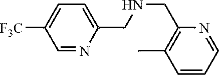 [SMILES: Cc1cccnc1CNCc1ccc(C(F)(F)F)cn1] | 1-(3-methylpyridin-2-yl)-N-((5-(trifluoromethyl)pyridin-2-yl)methyl)methanamine | 282.0 |
| 49 | 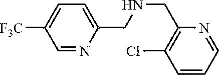 [SMILES: FC(F)(F)c1ccc(CNCc2ncccc2Cl)nc1] | 1-(3-chloropyridin-2-yl)-N-((5-(trifluoromethyl)pyridin-2-yl)methyl)methanamine | 302.2 |
| 50 | 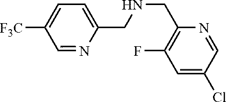 [SMILES: Fc1cc(Cl)cnc1CNCc1ccc(C(F)(F)F)cn1] | 1-(5-chloro-3-fluoropyridin-2-yl)-N-((5-(trifluoromethyl)pyridin-2-yl)methyl)methanamine | 320.1 |
| 51 | 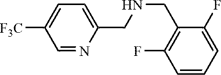 [SMILES: Fc1cccc(F)c1CNCc1ccc(C(F)(F)F)cn1] | N-(2,6-difluorobenzyl)-1-(5-(trifluoromethyl)pyridin-2-yl)methanamine | 302.9 |
| 52 | 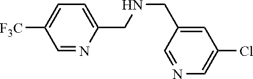 [SMILES: FC(F)(F)c1ccc(CNCc2cncc(Cl)c2)nc1] | 1-(5-chloropyridin-3-yl)-N-((5-(trifluoromethyl)pyridin-2-yl)methyl)methanamine | 302.3 |
| 53 | 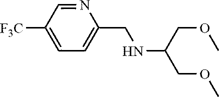 [SMILES: COCC(COC)NCc1ccc(C(F)(F)F)cn1] | 1,3-dimethoxy-N-((5-(trifluoromethyl)pyridin-2-yl)methyl)propan-2-amine | 279.2 |
| 54 | 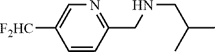 | N-((5-(difluoromethyl)pyridin-2-yl)methyl)-2-methylpropan-1-amine | 215.1 |
| 55 | 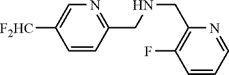 [SMILES: Fc1cccnc1CNCc1ccc(C(F)F)cn1] | 1-(5-(difluoromethyl)pyridin-2-yl)-N-((3-fluoropyridin-2-yl)methyl)methanamine | 268.2 |
| 56 | 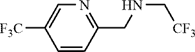 [SMILES: FC(F)(F)CNCc1ccc(C(F)(F)F)cn1] | 2,2,2-trifluoro-N-((5-(trifluoromethyl)pyridin-2-yl)methyl)ethan-1-amine | 259.0 |
| 57 | 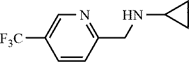 [SMILES: FC(F)(F)c1ccc(CNC2CC2)nc1] | N-((5-(trifluoromethyl)pyridin-2-yl)methyl)cyclopropanamine | 217 |
| 58 | 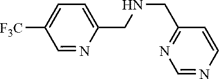 [SMILES: FC(F)(F)c1ccc(CNCc2ccncn2)nc1] | 1-(pyrimidin-4-yl)-N-((5-(trifluoromethyl)pyridin-2-yl)methyl)methanamine | 268.9 |
| 59 | 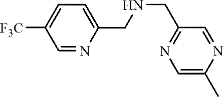 [SMILES: Cc1cnc(CNCc2ccc(C(F)(F)F)cn2)cn1] | 1-(5-methylpyrazin-2-yl)-N-((5-(trifluoromethyl)pyridin-2-yl)methyl)methanamine | 283.2 |
| 60 | 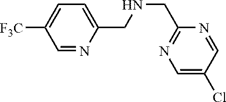 [SMILES: FC(F)(F)c1ccc(CNCc2ncc(Cl)cn2)nc1] | 1-(5-chloropyrimidin-2-yl)-N-((5-(trifluoromethyl)pyridin-2-yl)methyl)methanamine | 303.2 |
| 61 | 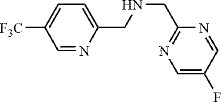 [SMILES: Fc1cnc(CNCc2ccc(C(F)(F)F)cn2)nc1] | 1-(5-fluoropyrimidin-2-yl)-N-((5-(trifluoromethyl)pyridin-2-yl)methyl)methanamine | 287.0 |
| 62 | 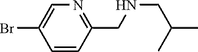 | N-((5-bromopyridin-2-yl)methyl)-2-methylpropan-1-amine | 243/245 |
| 63 | 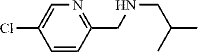 | N-((5-chloropyridin-2-yl)methyl)-2-methylpropan-1-amine | 199.1 |
| 64 | 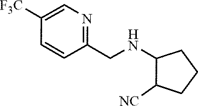 [SMILES: N#CC1CCCC1NCc1ccc(C(F)(F)F)cn1] | 2-(((5-(trifluoromethyl)pyridin-2-yl)methyl)amino)cyclopentane-1-carbonitrile | 270.2 |

Intermediate 65: 6-((((3-fluoropyridin-2-yl)methyl)amino)methyl)nicotinonitrile.

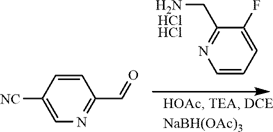

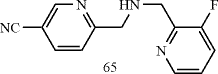

A mixture of 6-formylnicotinonitrile (0.198 g, 1.49 mmol, Enamine), 1,2-dichloroethane (7 mL), (3-fluoropyridin-2-yl)methanamine dihydrochloride (0.328 g, 1.64 mmol, Aurum Pharmatech) and TEA (0.47 mL, 3.37 mmol) was stirred at RT for 2 min, then acetic acid (0.10 mL, 1.72 mmol) was added. After 10 min, sodium triacetoxyborohydride (0.41 g, 1.94 mmol) was added. The mixture was stirred at RT for 2.5 h then neutralized with saturated aqueous Na₂CO₃. The mixture was extracted with DCM three times. The organic phase was dried over Na₂SO₄ and concentrated in vacuo. The crude was purified by silica gel chromatography (0-100% EtOAc/EtOH (3/1) in heptane) to afford the title compound (65, 0.24 g, 66% yield) an orange oil. MS: m/z (ESI): 243 (M+H)⁺.

TABLE 3

Secondary amines 66 to 75 were prepared in a manner similar to that described for Intermediate 65

| Int. # | Chemical Structure | Name | m/z (ESI: (M+H)⁺ |
|---|---|---|---|
| 66 | | 1-(3-fluoropyridin-2-yl)-N-(4-(trifluoromethyl)benzyl)methanamine | 285 |
| 67 | | 1-(3-fluoropyridin-2-yl)-N-(2-methyl-4-(trifluoromethyl)benzyl)methanamine | 299 |
| 68 | | methyl 6-((((3-fluoropyridin-2-yl)methyl)amino)methyl)nicotinate | 276 |
| 69 | | 1-(3-fluoropyridin-2-yl)-N-((6-(trifluoromethyl)pyridin-3-yl)methyl)methanamine | 286.1 |
| 70 | | 6-(((cyclopropylmethyl)amino)methyl)nicotinonitrile | 188 |
| 71 | | 1-(5-chloropyridin-2-yl)-N-(pyrimidin-2-ylmethyl)methanamine | 235.2 |
| 72 | | 1-(pyrimidin-2-yl)-N-((5-(trifluoromethyl)pyridin-2-yl)methyl)cyclopropan-1-amine | 295.2 |
| 73 | | N-((3,4-dihydro-2H-pyrano[2,3-c]pyridin-6-yl)methyl)-1-(pyrimidin-2-yl)ethan-1-amine | 271.1 |
| 74 | | N-(isoquinolin-3-ylmethyl)-1-(pyrimidin-2-yl)ethan-1-amine | 265.1 |
| 75 | 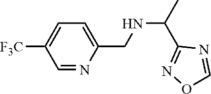 [SMILES: CC(NCc1ccc(C(F)(F)F)cn1)c1ncon1] | 1-(1,2,4-oxadiazol-3-yl)-N-((5-(trifluoromethyl)pyridin-2-yl)methyl)ethan-1-amine | 273 |

Intermediate 76: 6-(1-(((5-(trifluoromethyl)pyridin-2-yl)methyl)amino)ethyl)picolinonitrile.

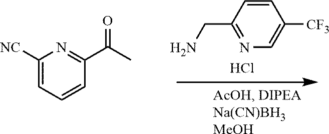

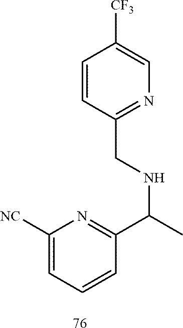

To a 100-mL round-bottomed flask was added 6-acetyl-2-cyanopyridine (0.500 g, 0.50 mL, 3.42 mmol, AstaTech, Inc) and (5-(trifluoromethyl)pyridin-2-yl)methanamine hydrochloride (0.73 g, 3.42 mmol, PharmaBlock Sciences, Inc) in methanol (17 mL). The reaction mixture was cooled to 0° C. then N,N′-diisopropylethylamine (0.66 g, 0.9 mL, 5.13 mmol, Sigma-Aldrich Corporation) was added to the reaction mixture, followed by acetic acid (0.2 mL, 3.42 mmol, Sigma-Aldrich Corporation) and sodium cyanoborohydride (0.65 g, 10.26 mmol, Oakwood Products, Inc.). The resulting reaction mixture was stirred at rt overnight. The reaction mixture was quenched with sat. aq. NaHCO₃, then the mixture was diluted with DCM. The layers were separated and the aqueous layer was extracted with DCM (3x). The combined organic extracts were dried over MgSO₄, filtered and concentrated in vacuo. The crude material was absorbed onto a plug of silica gel and purified by chromatography through a Redi-Sep pre-packed silica gel column (80 g), eluting with a gradient of 0-80% EtOAc:EtOH (3:1) in heptane, to afford 6-(1-(((5-(trifluoromethyl)pyridin-2-yl)methyl)amino)ethyl)picolinonitrile (0.26 g, 0.86 mmol, 25% yield) as tan oil. ¹H NMR (400 MHz, DMSO-d₆) δ ppm 8.83 (s, 1H), 8.14 (dd, J=8.4, 2.1 Hz, 1 H), 8.01 (t, J=7.5 Hz, 1 H), 7.89 (dd, J=7.6, 0.9 Hz, 1 H), 7.83 (dd, J=7.9, 0.8 Hz, 1 H), 7.68 (d, J=8.2 Hz, 1 H), 3.85 - 3.93 (m, 1 H), 3.75 - 3.79 (m, 2 H), 3.06 - 3.17 (m, 1 H), 1.33 (d, J=6.9 Hz, 3 H). m/z (ESI): 307.0 (M+H)⁺.

Intermediate 77: (R)-1-(pyrimidin-2-yl)-N-((6-(2,2,2-trifluoroethoxy)pyridazin-3-yl)methyl)ethan-1-amine.

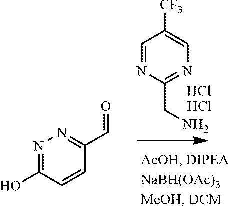

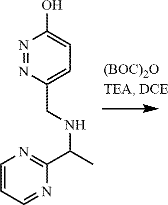

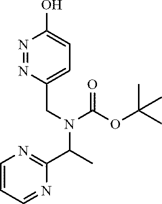 [SMILES: CC(c1ncccn1)N(Cc1ccc(O)nn1)C(=O)OC(C)(C)C]

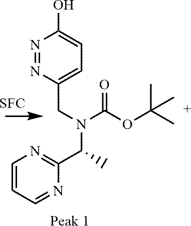

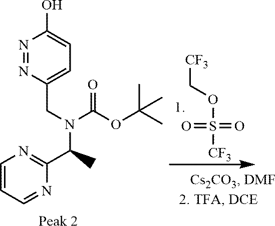

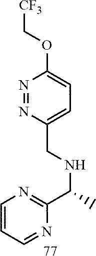

To a 150-mL round-bottomed flask was added 1-(pyrimidin-2-yl)ethan-1-amine dihydrochloride (2.79 g, 14.23 mmol, Enamine) in a 1:1 mixture of methanol (22 mL)/dichloromethane (22 mL). The reaction mixture was cooled to 0° C., and N,N′-diisopropylethylamine (3.51 g, 4.7 mL, 27.2 mmol, Sigma-Aldrich Corporation) was added to the reaction mixture and stirred 10 min. Then, 3-formyl-6-hydroxypyridazine (1.6 g, 12.93 mmol, Aurum Pharmatech LLC) and acetic acid (0.78 g, 0.8 mL, 12.93 mmol, Sigma-Aldrich Corporation) were added to the mixture, followed by sodium triacetoxyborohydride (12.93 mmol, Sigma-Aldrich Corporation). The reaction mixture was warmed to rt over 15 min, then sodium triacetoxyborohydride (6.85 g, 32.3 mmol, Sigma-Aldrich Corporation) was added to the reaction mixture and the overall mixture was stirred for 16 h, while under an inert (N₂) atmosphere. Another aliquot of sodium triacetoxyborohydride (6.85 g, 32.3 mmol, Sigma-Aldrich Corporation) was added to the reaction mixture and stirred an additional 16 h. The reaction mixture was filtered through a pad of Celite, then the filter cake was rinsed with 1:1 MeOH:DCM (3x). The filtrate was collected and concentrated in vacuo. The crude material was absorbed onto a plug of silica gel and purified by chromatography through a Redi-Sep pre-packed silica gel column (120 g), eluting with a gradient of 0-35% MeOH in CH₂Cl₂, to afford 6-(((1-(pyrimidin-2-yl)ethyl)amino)methyl)pyridazin-3-ol (1.12 g, 4.84 mmol, 37% yield) as light-yellow solid. ¹H NMR (400 MHz, METHANOL-d₄) δ ppm 8.78 (d, J=5.0 Hz, 2 H), 7.54 (d, J=9.6 Hz, 1 H), 7.38 (t, J=4.9 Hz, 1 H), 6.92 (d, J=9.6 Hz, 1 H), 4.07 (q, J=6.8 Hz, 1 H), 3.72 (d, J=2.3 Hz, 2 H), 1.98 (s, 1 H), 1.48 (d, J=6.9 Hz, 3 H). m/z (ESI): 232.1 (M+H)⁺.

To a 150-mL round-bottomed flask was added 6-(((1-(pyrimidin-2-yl)ethyl)amino)methyl)pyridazin-3-ol (1.11 g, 4.80 mmol) and triethylamine (2 mL, 14.40 mmol, Sigma-Aldrich Corporation) in 1,2-dichloroethane (24 mL). Then di-tert-butyl dicarbonate (1.57 g, 7.20 mmol, Sigma-Aldrich Corporation) was added to the reaction mixture. The overall reaction mixture was stirred and heated at 70° C. for 2 h. The reaction mixture was quenched with sat. aq. NaHCO₃ then the mixture was diluted with DCM. The layers were separated and the aqueous layer was extracted with DCM (3x). The combined organic extracts were dried over MgSO₄, filtered and concentrated in vacuo. The crude material was absorbed onto a plug of silica gel and purified by chromatography through a Redi-Sep pre-packed silica gel column (120 g), eluting with a gradient of 0-80% EtOAc:EtOH (3:1) in heptane, to provide tert-butyl ((6-hydroxypyridazin-3-yl)methyl)(1-(pyrimidin-2-yl)ethyl)carbamate (1.04 g, 3.15 mmol, 66% yield) as white solid. ¹H NMR (400 MHz, DMSO-d₆) δ ppm 12.77 (s, 1 H), 8.75 (d, J=4.8 Hz, 2 H), 7.37 (t, J=4.8 Hz, 2 H), 6.85 (d, J=9.6 Hz, 1 H), 4.88 - 5.05 (m, 1 H), 4.42 (br s, 2 H), 1.54 (d, J=7.3 Hz, 3 H), 1.15 - 1.34 (m, 9 H). m/z 332.1 (ESI): (M+H)⁺.

Racemic tert-butyl ((6-hydroxypyridazin-3-yl)methyl)(1-(pyrimidin-2-yl)ethyl)carbamate (1.0 g) was purified via preparative SFC using a Chiral Technologies AD column (250 × 30 mm, 5 mm) with a mobile phase of 80% Liquid CO₂ and 20% EtOH with 0.2% TEA using a flowrate of 150 mL/min. to generate peak 1 tert-butyl (R)-((6-hydroxypyridazin-3-yl)methyl)(1-(pyrimidin-2-yl)ethyl)carbamate (0.436 g, ee >99%) and peak 2 tert-butyl (S)-((6-hydroxypyridazin-3-yl)methyl)(1-(pyrimidin-2-yl)ethyl)carbamate (0.455 g, ee 98.8%). Peak assignment determined by SFC with AD column with 10% EtOH with 0.2% TEA.

A resealable vial was charged with tert-butyl (R)-((6-hydroxypyridazin-3-yl)methyl)(1-(pyrimidin-2-yl)ethyl)carbamate (0.075 g, 0.23 mmol), 2,2,2-trifluoroethyl triflate (0.07 g, 0.04 mL, 0.28 mmol, Combi-Blocks Inc.) and cesium carbonate (0.09 g, 0.28 mmol, Sigma-Aldrich Corporation) in N, N-dimethylformamide (2.3 mL). The vial was sealed and the reaction mixture was stirred at rt overnight. The reaction mixture was concentrated in vacuo. The crude material was absorbed onto a plug of silica gel and purified by chromatography through a Redi-Sep pre-packed silica gel column (12 g), eluting with a gradient of 0-60% MeOH in CH₂Cl₂, to provide tert-butyl (S)-(1-(pyrimidin-2-yl)ethyl)((6-(2,2,2-trifluoroethoxy)pyridazin-3-yl)methyl)carbamate (0.075 g, 0.18 mmol, 80% yield) as light-yellow solid. m/z 414.2 (ESI): (M+H)⁺.

To a 50-mL round-bottomed flask was added tert-butyl (R)-(1-(pyrimidin-2-yl)ethyl)((6-(2,2,2-trifluoroethoxy)pyridazin-3-yl)methyl)carbamate (0.075 g, 0.18 mmol) and trifluoroacetic acid (0.47 mL, 6.35 mmol, Sigma-Aldrich Corporation) in 1,2-dichloroethane (1.8 mL). The reaction mixture was stirred at 70° C. for 6 h. The reaction mixture was concentrated in vacuo. This afforded (R)-1-(pyrimidin-2-yl)-N-((6-(2,2,2-trifluoroethoxy)pyridazin-3-yl)methyl)ethan-1-amine as a crude residue, which was used immediately in the next step of the synthesis. m/z 314.0 (ESI): (M+H)⁺.

Intermediate 78: N-((6-(difluoromethoxy)pyridazin-3-yl)methyl)-1-(pyrimidin-2-yl)ethan-1-amine.

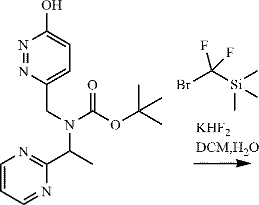

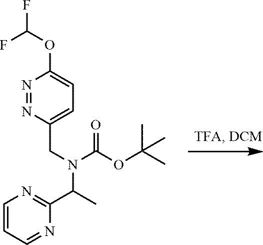

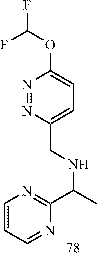

To a 50-mL round-bottomed flask was added tert-butyl ((6-hydroxypyridazin-3-yl)methyl)(1-(pyrimidin-2-yl)ethyl)carbamate (0.40 g, 1.21 mmol) and potassium bifluoride (0.75 g, 9.66 mmol, Sigma-Aldrich Corporation) in dichloromethane (2.8 mL)/water (1.21 mL). The reaction mixture was sparged with Argon (gas) for 5 min. Then, trimethyl(bromodifluoromethyl)silane (0.98 g, 4.83 mmol, Combi-Blocks Inc.) was added to the reaction mixture. The overall reaction mixture was stirred at rt overnight. The reaction mixture was poured into ice water, then the mixture was treated with sat. aq. NaHCO₃. The layers were separated and the aqueous layer was extracted with DCM (3x). The organic extracts were combined and dried over MgSO₄, filtered and concentrated in vacuo. This provided tert-butyl ((6-(difluoromethoxy)pyridazin-3-yl)methyl)(1-(pyrimidin-2-yl)ethyl)carbamate as crude oil, which was used directly in next step of synthesis without further purification. m/z 382.1 (ESI): (M+H)⁺.

The previous residue was diluted with dichloromethane (2.8 mL), then treated with trifluoracetic acid (4.8 g, 3.2 mL, 42.2 mmol, Sigma-Aldrich Corporation). The reaction mixture and stirred at rt for 2 h. The reaction mixture was concentrated in vacuo. The residue was diluted with DCM and treated with sat. aq. NaHCO₃. The layers were separated and the aqueous layer was extracted with DCM (3x). The combined organic extracts were dried over MgSO₄, filtered and concentrated in vacuo. The crude material was absorbed onto a plug of silica gel and purified by chromatography through a Redi-Sep pre-packed silica gel column (40 g), eluting with a gradient of 0-100% EtOAc:EtOH (3:1) in heptane, to provide N-((6-(difluoromethoxy)pyridazin-3-yl)methyl)-1-(pyrimidin-2-yl)ethan-1-amine (0.100 g, 0.356 mmol, 30% yield) as tan solid. m/z (ESI): 282.2 (M+H)⁺.

Intermediate 79: 1-(pyrimidin-2-yl)-N-((6-(trifluoromethoxy)pyridazin-3-yl)methyl)ethan-1-amine.

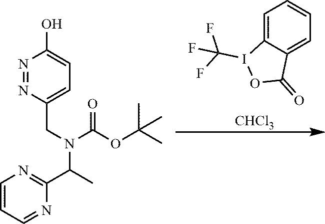

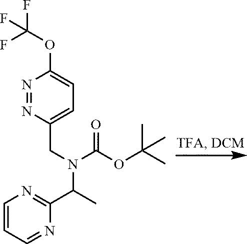

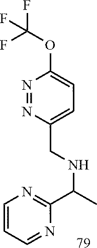

To a 50-mL round-bottomed flask was added tert-butyl ((6-hydroxypyridazin-3-yl)methyl)(1-(pyrimidin-2-yl)ethyl)carbamate (0.20 g, 0.60 mmol) and 1-trifluoromethyl-1,2-benziodoxol-3-(1h)-one (Togni’s Reagent 1) (1.59 g, 3.02 mmol, Sigma-Aldrich Corporation) in chloroform (6 mL). The reaction mixture was stirred and heated at 70° C. for 2 days. The reaction mixture was filtered through a pad of celite, then the filtrate was collected and treated with sat.aq. NaHCO₃. The aqueous layer was extracted with DCM (3x). The combined organic extracts were dried over MgSO₄, filtered and concentrated in vacuo. The crude material was absorbed onto a plug of silica gel and purified by chromatography through a Redi-Sep pre-packed silica gel column (12 g), eluting with a gradient of 0-75% EtOAc:EtOH (3:1) in heptane, to provide tert-butyl (1-(pyrimidin-2-yl)ethyl)((6-(trifluoromethoxy)pyridazin-3-yl)methyl)carbamate (0.040 g, 0.100 mmol, 17% yield) as tan solid. m/z (ESI): 400.1 (M+H)⁺.

To a 50-mL round-bottomed flask was added tert-butyl (1-(pyrimidin-2-yl)ethyl)((6-(trifluoromethoxy)pyridazin-3-yl)methyl)carbamate (0.040 g, 0.100 mmol) and trifluoracetic acid (0.3 mL, 3.51 mmol, Sigma-Aldrich Corporation) in dichloromethane (1 mL). The resulting reaction mixture was stirred at rt for 1 h. then concentrated in vacuo. The crude material was used immediately in next step of the synthesis, without further purification. m/z (ESI): 300.1 (M+H)⁺.

Intermediate 80: 6-fluoro-N-((5-(trifluoromethyl)pyridin-2-yl)methyl)-3,4-dihydro-2H-pyrano[3,2-b]pyridin-4-amine.

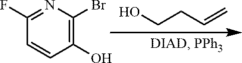

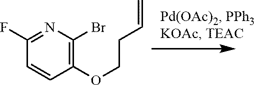

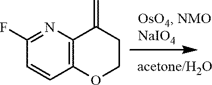

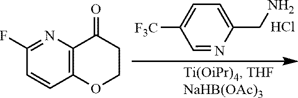

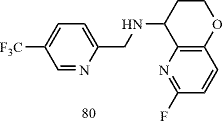

Triphenylphosphine (1.37 g, 5.21 mmol, Sigma-Aldrich) and DIAD (1.05 g, 1.03 mL, 5.21 mmol, Sigma-Aldrich) were added to a solution of 3-buten-1-ol (0.38 g, 0.45 mL, 5.21 mmol, Sigma-Aldrich) and 2-bromo-6-fluoro-3-hydroxypyridine (1.0 g, 5.21 mmol, Combi-Blocks Inc.) in THF (20 mL) under argon at 0° C. The ice bath was removed, and the mixture was stirred at 55° C. for 18 h, then cooled to RT. The organic material was fused to silica gel then purified by chromatography through a Redi-Sep pre-packed silica gel column (0-40% EtOAc/heptane) to provide 2-Bromo-3-(but-3-en-1-yloxy)-6-fluoropyridine (1.12 g, 4.55 mmol, 87% yield) as a colorless oil. m/z (ESI): 245.9/247.9 (M+H)⁺.

A mixture of 2-bromo-3-(but-3-en-1-yloxy)-6-fluoropyridine (1.12 g, 4.55 mmol), palladium acetate (0.102 g, 0.46 mmol), triphenylphosphine (0.36 g, 1.37 mmol, Sigma-Aldrich), potassium acetate (2.23 g, 22.8 mmol, Sigma-Aldrich Corporation), tetraethylammonium chloride (1.51 g, 9.10 mmol, Sigma-Aldrich Corporation), and water (0.16 g, 0.16 mL, 9.10 mmol) in DMF (20 mL) under argon was stirred at 100° C. for 6 h then cooled to RT. The resulting mixture was filtered through celite and the filtrate was diluted with EtOAc (50 mL). This mixture was washed with water (3 × 20 mL), dried over anhydrous MgSO₄, filtered, and concentrated in vacuo to give 6-Fluoro-4-methylene-3,4-dihydro-2H-pyrano[3,2-b]pyridine as an oil, which was taken directly to the next step without further purification. m/z (ESI): 166.2 (M+H)⁺.

Osmium tetroxide solution (0.46 g, 0.57 mL, 0.046 mmol, Sigma-Aldrich) was added to a solution of 6-fluoro-4-methylene-3,4-dihydro-2H-pyrano[3,2-b]pyridine (4.55 mmol) and 4-methylmorpholine N-oxide (0.64 g, 5.46 mmol, Sigma-Aldrich) in acetone (20 mL) and water (2.9 mL). This mixture was stirred for 6 h at RT before sodium (meta)periodate (2.44 g, 11.4 mmol, Sigma-Aldrich) was added. This mixture was stirred for 18 h at RT and then EtOAc (50 mL) and saturated sodium thiosulfate (50 mL) were added. This mixture was stirred vigorously for 5 min and then the layers were separated. The organic layer was then dried over anhydrous MgSO₄, filtered, and concentrated in vacuo to give an oil. The oil was purified by silica gel chromatography (0-100% EtOAc/heptane) to give 6-fluoro-2,3-dihydro-4H-pyrano[3,2-b]pyridin-4-one (0.58 g, 3.47 mmol, 76% yield for 2 steps) as a white solid. ¹H NMR (400 MHz, DMSO-d6) δ ppm 7.78 (dd, J=8.9, 6.6 Hz, 1 H), 7.41 (dd, J=8.9, 3.7 Hz, 1H), 4.59 - 4.65 (m, 2 H), 2.85 - 2.92 (m, 2 H). m/z (ESI): 168.2 (M+H)⁺

Titanium isopropoxide (0.34 g, 0.35 mL, 1.20 mmol, Sigma-Aldrich) was added to a solution of 6-fluoro-2,3-dihydro-4H-pyrano[3,2-b]pyridin-4-one (0.20 g, 1.2 mmol) and (5-(trifluoromethyl)pyridin-2-yl)methanamine hydrochloride (254 mg, 1.20 mmol, PharmaBlock) in THF (4 mL). The mixture was stirred under argon atmosphere for 18 h then sodium triacetoxyborohydride (507 mg, 2.39 mmol, Sigma-Aldrich) was added. This mixture was stirred for 4 h at RT, then 1 mL of NH₄OH (aq) was added. The resulting suspension was filtered, and the filtrate was concentrated in vacuo and then purified by silica gel chromatography (0-10% 2 N NH₃-MeOH/DCM gradient) to provide 6-fluoro-N-((5-(trifluoromethyl)pyridin-2-yl)methyl)-3,4-dihydro-2H-pyrano[3,2-b]pyridin-4-amine (246 mg, 0.75 mmol, 63% yield) as a light-yellow oil. m/z (ESI): 328.0 (M+H)⁺.

Intermediate 81: 6-fluoro-N-((6-(trifluoromethyl)pyridazin-3-yl)methyl)-3,4-dihydro-2H-pyrano[3,2-b] pyridin-4-amine.

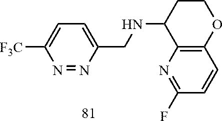

This amine was prepared in a manner similar to that described for amine 80. m/z (ESI): 329.2 (M+H)⁺.

Intermediate 82: rac-2-methyl-1-(pyrimidin-2-yl)-N-((5-(trifluoromethyl)pyridin-2-yl)methyl)propan-1-amine.

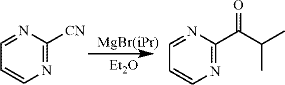

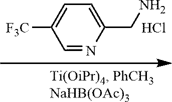

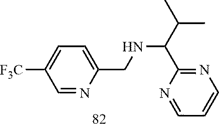

Isopropylmagnesium bromide (15.22 mL, 11.42 mmol) was added dropwise to a solution of pyrimidine-2-carbonitrile (1 g, 9.51 mmol, Combi-Blocks Inc.) in diethyl ether (15 mL) at -15° C., and the resulting mixture was stirred at -15° C. for 1 h, then at ambient temperature for 2 h. The reaction mixture was poured into ice water, and saturated aq. sodium bicarbonate solution was added to bring the final pH to 8. The resulting mixture was extracted with DCM (2 × 25 mL), and the combined organic layers were washed with brine (25 mL), dried over anhydrous sodium sulfate, filtered, and concentrated in vacuo. The resulting oil was absorbed onto silica gel and purified by silica gel chromatography (0-30% ethyl acetate/pet. ether) to give 2-methyl-1-(pyrimidin-2-yl)propan-1-one (0.7 g, 4.47 mmol, 47% yield) as yellow oil. ¹H NMR (400 MHz, Chloroform-d): δ ppm 8.95 (d, J=4.9 Hz, 2 H), 7.47 (t, J=4.9 Hz, 1 H), 3.98 - 4.09 (m, 1 H), 1.28 (s, 3 H), 1.26 (s, 3 H). m/z (ESI): 151.1 (M+H)⁺.

Intermediate 82 was prepared from 2-methyl-1-(pyrimidin-2-yl)propan-1-one and (5-(trifluoromethyl)pyridin-2-yl)methanamine hydrochloride (Shanghai Arbor Chemical Co., Ltd.) in a manner similar to that described for amine 80. m/z (ESI): 311.1 (M+H)⁺.

Intermediate 83: N-((5-(trifluoromethyl)pyridin-2-yl)methyl)-3,4-dihydro-2H-pyrano[3,2-b]pyridin-4-amine

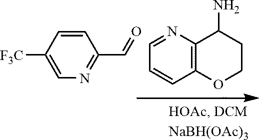

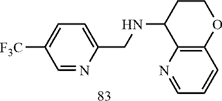

To a stirred mixture of 5-(trifluoromethyl)picolinaldehyde (554 mg, 3.16 mmol) and 2H,3H,4H-pyrano[3,2-b]pyridin-4-amine (500 mg, 3.33 mmol, AstaTech, Inc) in DCM (6 mL) in a 25 \-mL single-necked round-bottomed flask at RT was added acetic acid (0.19 mL, 3.33 mmol) followed by, 10 min later, sodium triacetoxyborohydride (1.05 g, 4.99 mmol) in one portion as a solid. The resulting mixture was stirred at RT for 3.5 h then poured into ice and saturated aqueous sodium carbonate and extracted with DCM (3 x). The combined organics were dried over anhydrous sodium sulfate and concentrated in vacuo. The residue was purified via silica gel chromatography (1-10% 2 M NH₃ in MeOH in DCM) to give the title amine (275 mg, 27% yield) as a colorless solid. m/z (ESI): 310.1 (M+H)⁺. ¹H NMR (CHLOROFORM-d, 400 MHz) δ 8.8-8.9 (m, 1H), 8.18 (dd, 1H, J=1.9, 4.0 Hz), 7.91 (dd, 1H, J=2.0, 8.3 Hz), 7.61 (d, 1H, J=8.4 Hz), 7.1-7.2 (m, 2H), 4.41 (m, 1H), 4.2-4.3 (m, 3H), 4.03 (t, 1H, J=5.7 Hz), 2.2-2.3 (m, 1H), 2.1-2.2 (m, 1H).

Table 4. Secondary amines below were prepared in a manner similar to that described for Intermediate 83. Compounds 87 and 88 were derived from (R)-5,6,7,8-tetrahydroquinolin-8-amine (purchased from Enamine) and (R)-5,6,7,8-tetrahydroisoquinolin-8-amine (purchased from Aurum), respectively. Compound 89 was derived from (R)-6,7-dihydro-5H-cyclopenta[b]pyridin-7-amine hydrochloride (AP Bioscience).

| Int. # | Chemical Structure | Name | m/z (ESI: (M+H)⁺ |
|---|---|---|---|
| 84 | 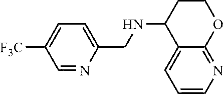 [SMILES: FC(F)(F)c1ccc(CNC2CCOc3ncccc32)nc1] | N-((5-(trifluoromethyl)pyridin-2-yl)methyl)-3,4-dihydro-2H-pyrano[2,3-b]pyridin-4-amine | 310.1 |
| 85 | 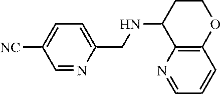 | 6-(((3,4-dihydro-2H-pyrano[3,2-b]pyridin-4-yl)amino)methyl)nicotinonitrile | 267.1 |
| 86 | 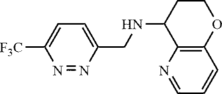 [SMILES: FC(F)(F)c1ccc(CNC2CCOc3cccnc32)nn1] | N-((6-(trifluoromethyl)pyridazin-3-yl)methyl)-3,4-dihydro-2H-pyrano[3,2-b]pyridin-4-amine | 311.1 |
| 87 | 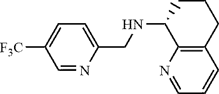 [SMILES: FC(F)(F)c1ccc(CN[C@@H]2CCCc3cccnc32)nc1] | (R)-N-((5-(trifluoromethyl)pyridin-2-yl)methyl)-5,6,7,8-tetrahydroquinolin-8-amine | 308.1 |
| 88 | 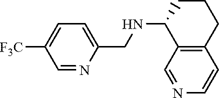 [SMILES: FC(F)(F)c1ccc(CN[C@@H]2CCCc3ccncc32)nc1] | (R)-N-((5-(trifluoromethyl)pyridin-2-yl)methyl)-5,6,7,8-tetrahydroisoquinolin-8-amine | 308.1 |
| 89 | 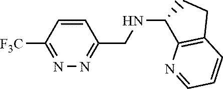 [SMILES: FC(F)(F)c1ccc(CN[C@@H]2CCc3cccnc32)nn1] | (R)-N-((6-(trifluoromethyl)pyridazin-3-yl)methyl)-6,7-dihydro-5H-cyclopenta[b]pyridin-7-amine | 295.0 |
| 90 | 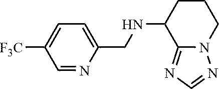 [SMILES: FC(F)(F)c1ccc(CNC2CCCn3ncnc32)nc1] | N-((5-(trifluoromethyl)pyridin-2-yl)methyl)-5,6,7,8-tetrahydro-[1,2,4]triazolo[1,5-a]pyridin-8-amine | 298.2 |
| 91 | 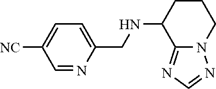 | 6-(((5,6,7,8-tetrahydro-[1,2,4]triazolo[1,5-a]pyridin-8-yl)amino)methyl)nicotinonitrile | 255.1 |
| 92 | 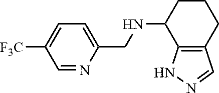 [SMILES: FC(F)(F)c1ccc(CNC2CCCc3cn[nH]c32)nc1] | N-((5-(trifluoromethyl)pyridin-2-yl)methyl)-4,5,6,7-tetrahydro-1H-indazol-7-amine | 297.2 |
| 93 | 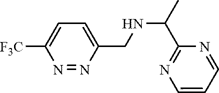 [SMILES: CC(NCc1ccc(C(F)(F)F)nn1)c1ncccn1] | (R)-1-(pyrimidin-2-yl)-N-((6-(trifluoromethyl)pyridazin-3-yl)methyl)ethan-1-amine | 284.2 |
| 94 | | (R)-4-chloro-6-(((1-(pyrimidin-2-yl)ethyl)amino)methyl)nicotinonitrile | 274.1 |
| 95 | 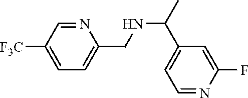 [SMILES: CC(NCc1ccc(C(F)(F)F)cn1)c1ccnc(F)c1] | 1-(2-fluoropyridin-4-yl)-N-((5-(trifluoromethyl)pyridin-2-yl)methyl)ethan-1-amine | 300.0 |
| 96 | 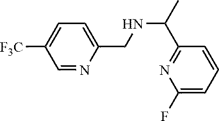 [SMILES: CC(NCc1ccc(C(F)(F)F)cn1)c1cccc(F)n1] | (R)-1-(6-fluoropyridin-2-yl)-N-((5-(trifluoromethyl)pyridin-2-yl)methyl)ethan-1-amine | 300.0 |
| 97 | 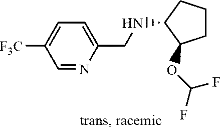 | (1R,2R)-2-(difluoromethoxy)-N-((5-(trifluoromethyl)pyridin-2-yl)methyl)cyclopentan-1-amine | 311.0 |
| 98 | 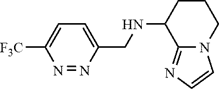 [SMILES: FC(F)(F)c1ccc(CNC2CCCn3ccnc32)nn1] | N-((6-(trifluoromethyl)pyridazin-3-yl)methyl)-5,6,7,8-tetrahydroimidazo[1,2-a]pyridin-8-amine | 298.2 |
| 99 | 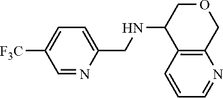 [SMILES: FC(F)(F)c1ccc(CNC2COCc3ncccc32)nc1] | N-((5-(trifluoromethyl)pyridin-2-yl)methyl)-5,8-dihydro-6H-pyrano[3,4-b]pyridin-5-amine | 310.2 |

Intermediate 100: N-((5-(trifluoromethyl)pyridin-2-yl)methyl)-5,6,7,8-tetrahydroquinoxalin-5-amine. This amine was prepared from 7,8-dihydroquinoxalin-5(6H)-one (purchased from Enamine) in a manner similar to that described for amine 65. m/z (ESI): 309.2 (M+H)⁺.

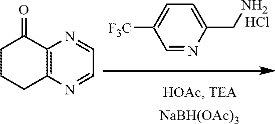

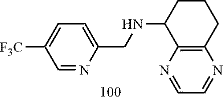

Intermediate 102: (R)-1-(pyrimidin-2-yl)-N-((5-(trifluoromethyl)pyridin-2-yl)methyl)ethan-1-amine.

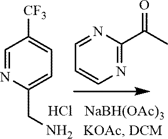

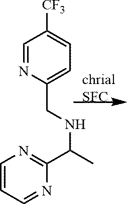

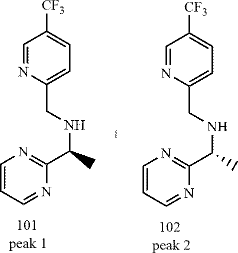

To a stirred solution of (5-(trifluoromethyl)pyridin-2-yl)methanamine hydrochloride (115 g, 541 mmol) and 1-(pyrimidin-2-yl)ethan-1-one (76 g, 622 mmol) in DCM (3.5 L) was added potassium acetate (63.7 g, 649 mmol). The mixture was stirred for 30 min then treated with sodium triacetoxyborohydride (149 g, 703 mmol). After stirring for 1.5 h, the reaction mixture was diluted with water (2 L), treated with 1 N HCl (2 L), and extracted with DCM (1 L). The layers were separated. The aqueous layer was treated with 10% sodium hydroxide to adjust the pH to 12 and extracted with DCM (3 × 2 L). The combined organic layer was dried over Na₂SO₄, filtered and concentrated. The crude material was purified by silica gel chromatography (2% MeOH in DCM) to give 1-(pyrimidin-2-yl)-N-((5-(trifluoromethyl)pyridin-2-yl)methyl)ethan-1-amine (97 g, 344 mmol, 63% yield) as a brown oil. m/z (ESI): 283.0 (M+H)⁺. ¹H NMR (400 MHz, Chloroform-d): δ ppm 8.81 (dt, J= 2.2, 1.0 Hz, 1 H), 8.74 (d, J = 4.9 Hz, 2 H), 7.88 (dd, J = 8.2, 2.3 Hz, 1 H), 7.54 (d, J = 8.2 Hz, 1 H), 7.20 (t, J = 4.9 Hz, 1 H), 4.10 (q, J = 6.8 Hz, 1 H), 3.94 (d,J= 2.9 Hz, 2 H), 1.53 (d, J= 6.8 Hz, 3 H).

The racemic secondary amine (44 g) was dissolved in 200 mL of MeOH and subjected to chiral SFC using following conditions. The 1^{st} eluting peak was (S)-1-(pyrimidin-2-yl)-N-((5-(trifluoromethyl)pyridin-2-yl)methyl)ethan-1-amine (101, 18 g, > 99% ee) and the 2^{nd} eluting peak was (R)-1-(pyrimidin-2-yl)-N-((5-(trifluoromethyl)pyridin-2-yl)methyl)ethan-1-amine (102, 19 g, > 99% ee). Column temperature = 40° C. Column = [Chiralpak AD-H (250×30 mm, 5 µ)]. Mobile phase = [Liquid CO₂: 0.5% DEA in EtOH (90:10)]. Flow rate= 100 mL/ min. Wavelength = 245 nm.

TABLE 5

Racemic amines below were prepared in a fashion similar to that described above for amine 102. The racemic amines were subjected to chiral SFC to provide enantiomerically pure amines (> 99% ee)

| Secondary Amines | SFC Conditions | m/z (ESI: (M+H)⁺ |
|---|---|---|
| 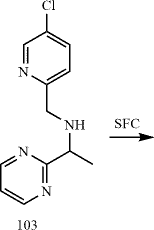 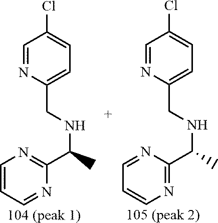 | Column: Chiralpak AD-H (250×30 mm, 5 µ). Mobile Phase: 75:25. B: 0.3% Et₂NH in EtOH. | 249.0 |
| 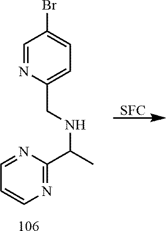 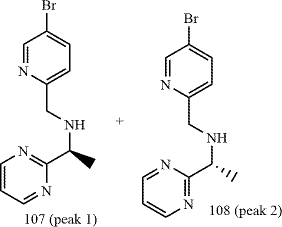 | Column: ChiralPak AD-H (250×30 mm, 5 µm). Mobile Phase: 70:30. B: 0.5% Et₂NH in EtOH. | 293/295 |
| 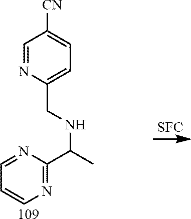 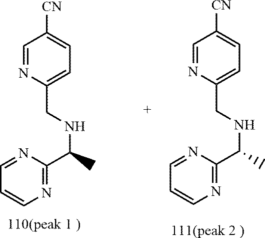 | Column: Chiral Technologies AZ (250× 21 mm, 5 µm). Mobile phase: 85:15. B: 0.2% TEA in MeOH. | 240.0 |
| 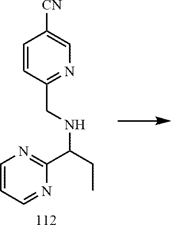 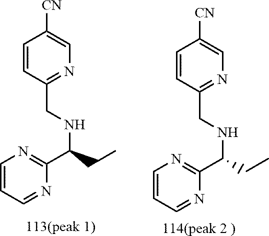 | Column: Chiral Technologies AZ column (250 × 21 mm, 5 mm) with a mobile phase of 90% Liquid CO2 and 10% EtOH with 0.2% TEA using a flowrate of 80 mL/min. | 254.0 |

Intermediate 115: (R)-N-((6-methoxypyridazin-3-yl)methyl)-1-(pyrimidin-2-yl)ethan-1-amine.

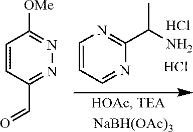

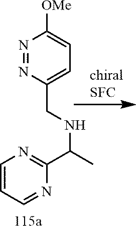

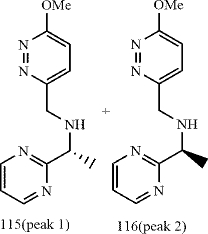

A mixture of 6-methoxy-pyridazine-3-carbaldehyde (0.49 g, 3.54 mmol, Princeton BioMolecular Research, Inc.), 1-(pyrimidin-2-yl)ethan-1-amine dihydrochloride (0.73 g, 3.72 mmol, Enamine), 1,2-dichloroethane (30 mL), and acetic acid (0.22 mL, 3.90 mmol) was stirred at RT for 10 min, then sodium triacetoxyborohydride (1.013 g, 4.78 mmol) was added. The mixture was stirred at RT for 30 min then neutralized with saturated Na₂CO₃ solution. The crude was extracted with DCM. The organic phase was dried over Na₂SO₄ and concentrated in vacuo. The residue was purified by silica gel chromatography (30-100% EtOAc/EtOH (3/1) in heptane) to provide N-((6-methoxypyridazin-3-yl)methyl)-1-(pyrimidin-2-yl)ethan-1-amine (0.84 g, 96% yield) as an orange oil. m/z (ESI): 246 (M+H)⁺.

The racemic amine from above was subjected to chiral SFC using a Chiral Technologies IC column (250 × 30 mm, 5 µm) with a mobile phase of 70% liquid CO₂ and 30% MeOH with 0.2% TEA using a flowrate of 150 mL/min. The 1^{st} eluting peak was (R)-N-((6-methoxypyridazin-3-yl)methyl)-1-(pyrimidin-2-yl)ethan-1-amine (115, 369 mg, > 99% ee) . The 2^{nd} eluting peak was (S)-N-((6-methoxypyridazin-3-yl)methyl)-1-(pyrimidin-2-yl)ethan-1-amine (116, 374 mg, > 99% ee).

TABLE 6

Racemic amines below were prepared in a fashion similar to that described above for amine 115. The racemic amines were subjected to chiral SFC to provide enantiomerically pure amines (> 99% ee)

| Secondary Amines | SFC Conditions | m/z (ESI: (M+H)⁺ |
|---|---|---|
| 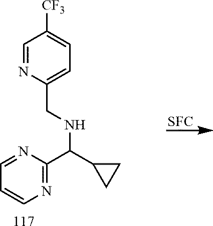 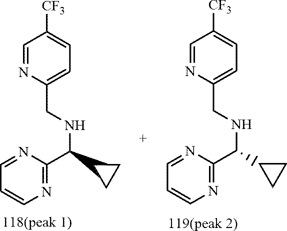 | Column: Chiral Technologies AZ (250× 21 mm, 5 µm). Mobile phase: 90:10. B: 0.2% TEA in iPrOH. | 309.0 |
| 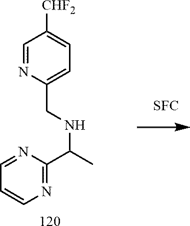 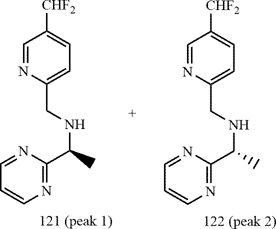 | Column: Chiral Technologies AD (250× 21 mm, 5 µm). Mobile phase: 85:15. B: 0.2% TEA in EtOH. | 265.1 |
| 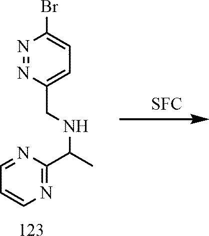 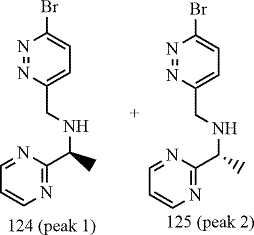 | Column: Chiral Technologies AD column (250 × 30 mm, 5 µm). Mobile phase: 70:30. B: 0.2% TEA in EtOH. | 293.9/ 295.9 |
| 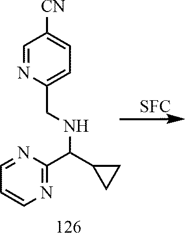 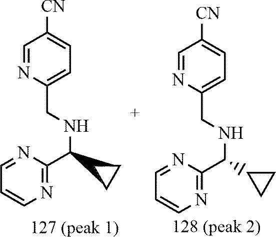 | Column: Chiral Technologies AZ (250 × 21 mm, 5 µm). Mobile phase: 75:25. B: 0.2% TEA in EtOH. | 266.0 |
| 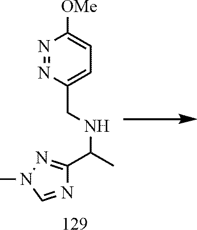 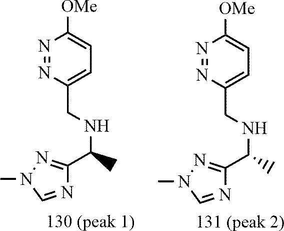 | Column: Chiral Technologies AD column (250 × 21 mm, 5 mm) with a mobile phase of 85% Liquid CO2 and 15% MeOH with 0.2% TEA using a flowrate of 100 mL/min. | 249 |
| 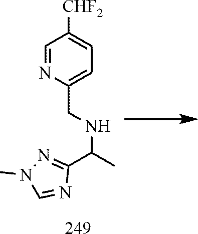 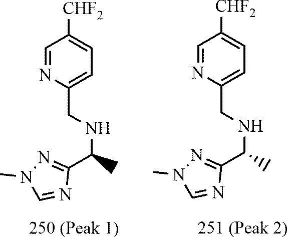 | Chiralpak IG 2×25 cm, 5 µm column; a mobile phase of 20% methanol with 1.0% DEA using a flowrate of 80 mL/min. | 268.0 |

TABLE 7

Secondary amines below were prepared in a manner similar to that described for amine 115a. Enantiopure amines were derived from commercially available enantiomerically pure reagents

| Int. # | Chemical Structure | Name | m/z (ESI: (M+H)⁺ |
|---|---|---|---|
| 132 | 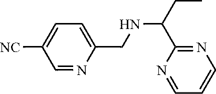 | 1-(pyrimidin-2-yl)-N-((5-(trifluoromethyl)pyrazin-2-yl)methyl)propan-1-amine | 298.0 |
| 133 | 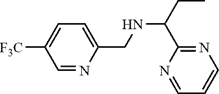 [SMILES: CCC(NCc1ccc(C(F)(F)F)cn1)c1ncccn1] | 1-(pyrimidin-2-yl)-N-((5-(trifluoromethyl)pyridin-2-yl)methyl)propan-1-amine | 297.1 |
| 134 | 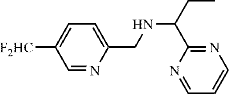 [SMILES: CCC(NCc1ccc(C(F)F)cn1)c1ncccn1] | N-((5-(difluoromethyl)pyridin-2-yl)methyl)-1-(pyrimidin-2-yl)propan-1-amine | 279.1 |
| 112 | 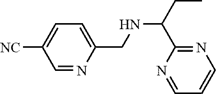 | -(((1-(pyrimidin-2-yl)propyl)amino)methyl)nicotinonitrile | 254.0 |
| 136 | 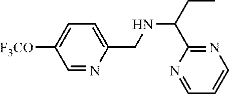 [SMILES: CCC(NCc1ccc(OC(F)(F)F)cn1)c1ncccn1] | 1-(pyrimidin-2-yl)-N-((5-(trifluoromethoxy)pyridin-2-yl)methyl)ethan-1-amine | 299.1 |
| 137 | 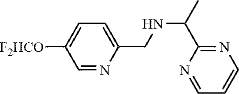 [SMILES: CC(NCc1ccc(OC(F)F)cn1)c1ncccn1] | N-((5-(difluoromethoxy)pyridin-2-yl)methyl)-1-(pyrimidin-2-yl)ethan-1-amine | 281.0 |
| 138 | 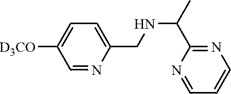 | N-((5-(methoxy-d3)pyridin-2-yl)methyl)-1-(pyrimidin-2-yl)ethan-1-amine | 248.0 |
| 139 | 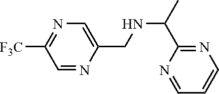 [SMILES: CC(NCc1cnc(C(F)(F)F)cn1)c1ncccn1] | 1-(pyrimidin-2-yl)-N-((5-(trifluoromethyl)pyrazin-2-yl)methyl)ethan-1-amine | 284.1 |
| 140 | 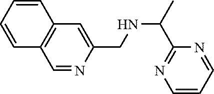 [SMILES: CC(NCc1cc2ccccc2cn1)c1ncccn1] | 1-(pyrimidin-2-yl)-N-(quinolin-3-ylmethyl)ethan-1-amine | 265.1 |
| 141 | 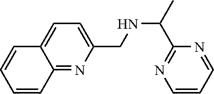 [SMILES: CC(NCc1ccc2ccccc2n1)c1ncccn1] | 1-(pyrimidin-2-yl)-N-(quinolin-2-ylmethyl)ethan-1-amine | 265.1 |
| 142 | 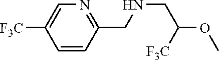 [SMILES: COC(CNCc1ccc(C(F)(F)F)cn1)C(F)(F)F] | 3,3,3-trifluoro-2-methoxy-N-((5-(trifluoromethyl)pyridin-2-yl)methyl)propan-1-amine | 303.0 |
| 143 | 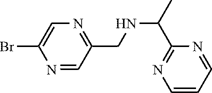 [SMILES: CC(NCc1cnc(Br)cn1)c1ncccn1] | N-((5-bromopyrazin-2-yl)methyl)-1-(pyrimidin-2-yl)ethan-1-amine | 294/296 |
| 144 | 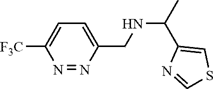 [SMILES: CC(NCc1ccc(C(F)(F)F)nn1)c1cscn1] | 1-(thiazol-4-yl)-N-((6-(trifluoromethyl)pyridazin-3-yl)methyl)ethan-1-amine | 289.0 |
| 145 | 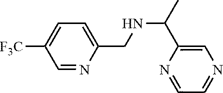 [SMILES: CC(NCc1ccc(C(F)(F)F)cn1)c1cnccn1] | 1-(pyrazin-2-yl)-N-((5-(trifluoromethyl)pyridin-2-yl)methyl)ethan-1-amine | 283.0 |
| 146 | 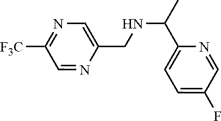 [SMILES: CC(NCc1cnc(C(F)(F)F)cn1)c1ccc(F)cn1] | 1-(5-fluoropyridin-2-yl)-N-((5-(trifluoromethyl)pyrazin-2-yl)methyl)ethan-1-amine | 301.0 |
| 147 | 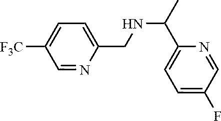 [SMILES: CC(NCc1ccc(C(F)(F)F)cn1)c1ccc(F)cn1] | 1-(5-fluoropyridin-2-yl)-N-((5-(trifluoromethyl)pyridin-2-yl)methyl)ethan-1-amine | 300.0 |
| 148 | 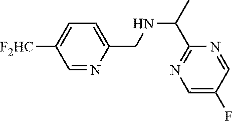 [SMILES: CC(NCc1ccc(C(F)F)cn1)c1ncc(F)cn1] | N-((5-(difluoromethyl)pyridin-2-yl)methyl)-1-(5-fluoropyrimidin-2-yl)ethan-1-amine | 283.0 |
| 149 | 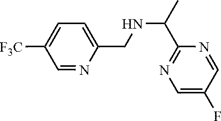 [SMILES: CC(NCc1ccc(C(F)(F)F)cn1)c1ncc(F)cn1] | 1-(5-fluoropyrimidin-2-yl)-N-((5-(trifluoromethyl)pyridin-2-yl)methyl)ethan-1-amine | 301.0 |
| 150 | 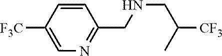 [SMILES: CC(CNCc1ccc(C(F)(F)F)cn1)C(F)(F)F] | 3,3,3-trifluoro-2-methyl-N-((5-(trifluoromethyl)pyridin-2-yl)methyl)propan-1-amine | 287.2 |
| 151 | 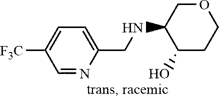 | (3S,4S)-3-(((5-(trifluoromethyl)pyridin-2-yl)methyl)amino)tetrahydro-2H-pyran-4-ol + enantiomer | 277.2 |
| 152 | 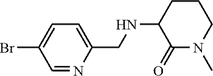 [SMILES: CN1CCCC(NCc2ccc(Br)cn2)C1=O] | 3-(((5-bromopyridin-2-yl)methyl)amino)-1-methylpiperidin-2-one | 298/300 |
| 153 | 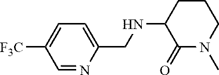 [SMILES: CN1CCCC(NCc2ccc(C(F)(F)F)cn2)C1=O] | 1-methyl-3-(((5-(trifluoromethyl)pyridin-2-yl)methyl)amino)piperidin-2-one | 288.2 |
| 154 | 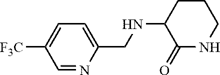 [SMILES: O=C1NCCCC1NCc1ccc(C(F)(F)F)cn1] | 3-(((5-(trifluoromethyl)pyridin-2-yl)methyl)amino)piperidin-2-one | 274.1 |
| 155 | 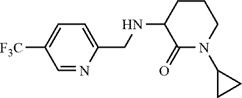 [SMILES: O=C1C(NCc2ccc(C(F)(F)F)cn2)CCCN1C1CC1] | 1-cyclopropyl-3-(((5-(trifluoromethyl)pyridin-2-yl)methyl)amino)piperidin-2-one | 314.1 |
| 156 | 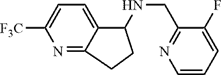 [SMILES: Fc1cccnc1CNC1CCc2nc(C(F)(F)F)ccc21] | N-((3-fluoropyridin-2-yl)methyl)-2-(trifluoromethyl)-6,7-dihydro-5H-cyclopenta[b]pyridin-5-amine | 312.0 |
| 157 | 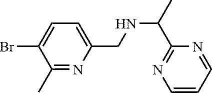 [SMILES: Cc1nc(CNC(C)c2ncccn2)ccc1Br] | N-((5-bromo-6-methylpyridin-2-yl)methyl)-1-(pyrimidin-2-yl)ethan-1-amine | 307/309 |
| 158 | 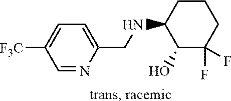 | 2,2-difluoro-6-(((5-(trifluoromethyl)pyridin-2-yl)methyl)amino)cyclohexan-1-ol | 311.1 |
| 159 | 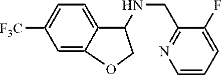 [SMILES: Fc1cccnc1CNC1COc2cc(C(F)(F)F)ccc21] | N-((3-fluoropyridin-2-yl)methyl)-6-(trifluoromethyl)-2,3-dihydrobenzofuran-3-amine | 313.2 |
| 160 | 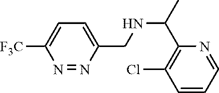 [SMILES: CC(NCc1ccc(C(F)(F)F)nn1)c1ncccc1Cl] | 1-(3-chloropyridin-2-yl)-N-((6-(trifluoromethyl)pyridazin-3-yl)methyl)ethan-1-amine | 317.2 |
| 161 | 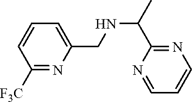 [SMILES: CC(NCc1cccc(C(F)(F)F)n1)c1ncccn1] | 1-(pyrimidin-2-yl)-N-((6-(trifluoromethyl)pyridin-2-yl)methyl)ethan-1-amine | 302.1 |
| 162 | 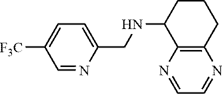 [SMILES: FC(F)(F)c1ccc(CNC2CCCc3nccnc32)nc1] | N-((5-(trifluoromethyl)pyridin-2-yl)methyl)-5,6,7,8-tetrahydroquinoxalin-5-amine | 309.2 |
| 163 | 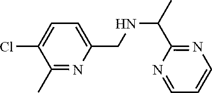 [SMILES: Cc1nc(CNC(C)c2ncccn2)ccc1Cl] | N-((5-chloro-6-methylpyridin-2-yl)methyl)-1-(pyrimidin-2-yl)ethan-1-amine | 263.2 |
| 164 | 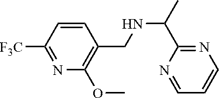 [SMILES: COc1nc(C(F)(F)F)ccc1CNC(C)c1ncccn1] | N-((2-methoxy-6-(trifluoromethyl)pyridin-3-yl)methyl)-1-(pyrimidin-2-yl)ethan-1-amine | 313.2 |
| 165 | 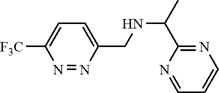 [SMILES: CC(NCc1ccc(C(F)(F)F)nn1)c1ncccn1] | 1-(pyrimidin-2-yl)-N-((6-(trifluoromethyl)pyridazin-3-yl)methyl)ethan-1-amine | 284.1 |
| 166 | 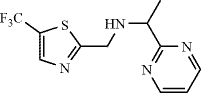 [SMILES: CC(NCc1ncc(C(F)(F)F)s1)c1ncccn1] | 1-(pyrimidin-2-yl)-N-((5-(trifluoromethyl)thiazol-2-yl)methyl)ethan-1-amine | 289.0 |
| 167 | 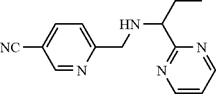 [SMILES: CCC(NCc1ccc(C#N)cn1)c1ncccn1] | 6-(((1-(pyrimidin-2-yl)propyl)amino)methyl)nicotinonitrile | 254.0 |
| 168 | 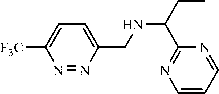 [SMILES: CCC(NCc1ccc(C(F)(F)F)nn1)c1ncccn1] | 1-(pyrimidin-2-yl)-N-((6-(trifluoromethyl)pyridazin-3-yl)methyl)propan-1-amine | 298.1 |
| 169 | 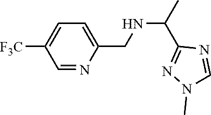 [SMILES: CC(NCc1ccc(C(F)(F)F)cn1)c1ncn(C)n1] | 1-(1-methyl-1H-1,2,4-triazol-3-yl)-N-((5-(trifluoromethyl)pyridin-2-yl)methyl)ethan-1-amine | 286.0 |
| 170 | 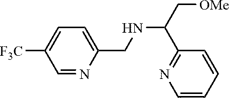 [SMILES: COCC(NCc1ccc(C(F)(F)F)cn1)c1ccccn1] | 2-methoxy-1-(pyridin-2-yl)-N-((5-(trifluoromethyl)pyridin-2-yl)methyl)ethan-1-amine | 312.0 |
| 171 | 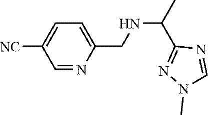 [SMILES: CC(NCc1ccc(C#N)cn1)c1ncn(C)n1] | 6-(((1-(1-methyl-1H-1,2,4-triazol-3-yl)ethyl)amino)methyl)nicotinonitrile | 243.0 |
| 172 | 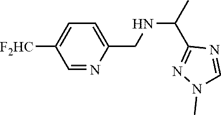 [SMILES: CC(NCc1ccc(C(F)F)cn1)c1ncn(C)n1] | N-((5-(difluoromethyl)pyridin-2-yl)methyl)-1-(1-methyl-1H-1,2,4-triazol-3-yl)ethan-1-amine | 268.0 |
| 173 | 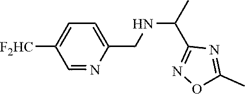 [SMILES: Cc1nc(C(C)NCc2ccc(C(F)F)cn2)no1] | N-((5-(difluoromethyl)pyridin-2-yl)methyl)-1-(5-methyl-1,2,4-oxadiazol-3-yl)ethan-1-amine | 269.2 |
| 174 | 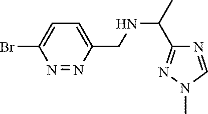 [SMILES: CC(NCc1ccc(Br)nn1)c1ncn(C)n1] | N-((6-bromopyridazin-3-yl)methyl)-1-(1-methyl-1H-1,2,4-triazol-3-yl)ethan-1-amine | 297/299 |
| 175 | 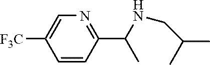 [SMILES: CC(C)CNC(C)c1ccc(C(F)(F)F)cn1] | 2-methyl-N-(1-(5-(trifluoromethyl)pyridin-2-yl)ethyl)propan-1-amine | 247.0 |
| 176 | 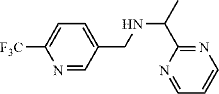 [SMILES: CC(NCc1ccc(C(F)(F)F)nc1)c1ncccn1] | 1-(pyrimidin-2-yl)-N-((6-(trifluoromethyl)pyridin-3-yl)methyl)ethan-1-amine | 283.0 |
| 177 | 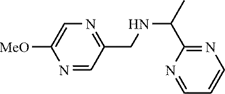 [SMILES: COc1cnc(CNC(C)c2ncccn2)cn1] | N-((5-methoxypyrazin-2-yl)methyl)-1-(pyrimidin-2-yl)ethan-1-amine | 246.0 |
| 178 | 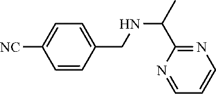 [SMILES: CC(NCc1ccc(C#N)cc1)c1ncccn1] | 4-(((1-(pyrimidin-2-yl)ethyl)amino)methyl)benzonitrile | 239.0 |
| 179 | 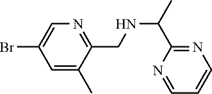 [SMILES: Cc1cc(Br)cnc1CNC(C)c1ncccn1] | N-((5-bromo-3-methylpyridin-2-yl)methyl)-1-(pyrimidin-2-yl)ethan-1-amine | 307/309 |
| 180 | 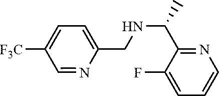 [SMILES: C[C@@H](NCc1ccc(C(F)(F)F)cn1)c1ncccc1F] | (R)-1-(3-fluoropyridin-2-yl)-N-((5-(trifluoromethyl)pyridin-2-yl)methyl)ethan-1-amine | 300.0 |
| 181 | 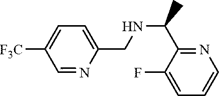 [SMILES: C[C@H](NCc1ccc(C(F)(F)F)cn1)c1ncccc1F] | (S)-1-(3-fluoropyridin-2-yl)-N-((5-(trifluoromethyl)pyridin-2-yl)methyl)ethan-1-amine | 300.0 |
| 182 | 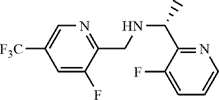 [SMILES: C[C@@H](NCc1ncc(C(F)(F)F)cc1F)c1ncccc1F] | (R)-N-((3-fluoro-5-(trifluoromethyl)pyridin-2-yl)methyl)-1-(3-fluoropyridin-2-yl)ethan-1-amine | 318.0 |
| 183 | 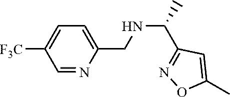 [SMILES: Cc1cc([C@@H](C)NCc2ccc(C(F)(F)F)cn2)no1] | (R)-1-(5-methylisoxazol-3-yl)-N-((5-(trifluoromethyl)pyridin-2-yl)methyl)ethan-1-amine | 286.1 |
| 184 | 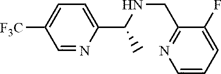 [SMILES: C[C@@H](NCc1ncccc1F)c1ccc(C(F)(F)F)cn1] | (R)-N-((3-fluoropyridin-2-yl)methyl)-1-(5-(trifluoromethyl)pyridin-2-yl)ethan-1-amine | 300.0 |
| 185 | 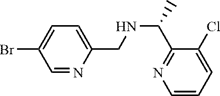 [SMILES: C[C@@H](NCc1ccc(Br)cn1)c1ncccc1Cl] | (R)-N-((5-bromopyridin-2-yl)methyl)-1-(3-chloropyridin-2-yl)ethan-1-amine | 326/328 |
| 186 | 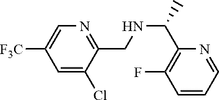 [SMILES: C[C@@H](NCc1ncc(C(F)(F)F)cc1Cl)c1ncccc1F] | (R)-N-((3-chloro-5-(trifluoromethyl)pyridin-2-yl)methyl)-1-(3-fluoropyridin-2-yl)ethan-1-amine | 334 |
| 187 | 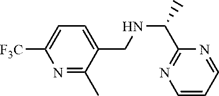 [SMILES: Cc1nc(C(F)(F)F)ccc1CN[C@H](C)c1ncccn1] | (R)-N-((2-methyl-6-(trifluoromethyl)pyridin-3-yl)methyl)-1-(pyrimidin-2-yl)ethan-1-amine | 297.2 |
| 188 | 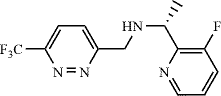 [SMILES: C[C@@H](NCc1ccc(C(F)(F)F)nn1)c1ncccc1F] | (R)-1-(3-fluoropyridin-2-yl)-N-((6-(trifluoromethyl)pyridazin-3-yl)methyl)ethan-1-amine | 301.0 |
| 189 | 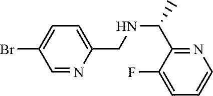 [SMILES: C[C@@H](NCc1ccc(Br)cn1)c1ncccc1F] | (R)-N-((5-bromopyridin-2-yl)methyl)-1-(3-fluoropyridin-2-yl)ethan-1-amine | 310/312 |
| 190 | 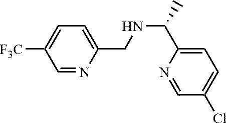 [SMILES: C[C@@H](NCc1ccc(C(F)(F)F)cn1)c1ccc(Cl)cn1] | (R)-1-(5-chloropyridin-2-yl)-N-((5-(trifluoromethyl)pyridin-2-yl)methyl)ethan-1-amine | 316.1 |
| 191 | 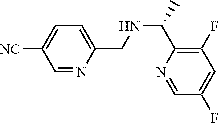 [SMILES: C[C@@H](NCc1ccc(C#N)cn1)c1ncc(F)cc1F] | (R)-6-(((1-(3,5-difluoropyridin-2-yl)ethyl)amino)methyl)nicotinonitrile | 275.0 |
| 192 | 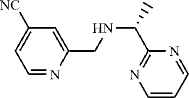 [SMILES: C[C@@H](NCc1cc(C#N)ccn1)c1ncccn1] | (R)-2-(((1-(pyrimidin-2-yl)ethyl)amino)methyl)isonicotinonitrile | 240 |
| 193 | 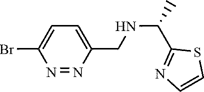 [SMILES: C[C@@H](NCc1ccc(Br)nn1)c1nccs1] | (R)-N-((6-bromopyridazin-3-yl)methyl)-1-(thiazol-2-yl)ethan-1-amine | 299/301 |
| 194 | 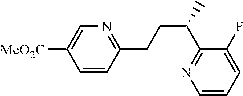 [SMILES: COC(=O)c1ccc(CC[C@H](C)c2ncccc2F)nc1] | methyl (R)-6-(((1-(3-fluoropyridin-2-yl)ethyl)amino)methyl)nicotinate | 290.0 |
| 195 | 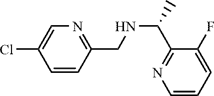 [SMILES: C[C@@H](NCc1ccc(Cl)cn1)c1ncccc1F] | (R)-N-((5-chloropyridin-2-yl)methyl)-1-(3-fluoropyridin-2-yl)ethan-1-amine | 266.0 |
| 196 | 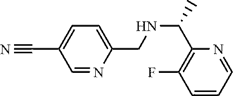 [SMILES: C[C@@H](NCc1ccc(C#N)cn1)c1ncccc1F] | (R)-6-(((1-(3-fluoropyridin-2-yl)ethyl)amino)methyl)nicotinonitrile | 257.0 |
| 197 | 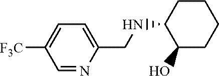 [SMILES: O[C@@H]1CCCC[C@H]1NCc1ccc(C(F)(F)F)cn1] | (1R,2R)-2-(((5-(trifluoromethyl)pyridin-2-yl)methyl)amino)cyclohexan-1-ol | 275.0 |
| 198 | 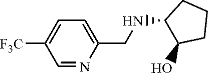 [SMILES: O[C@@H]1CCC[C@H]1NCc1ccc(C(F)(F)F)cn1] | (1R,2R)-2-(((5-(trifluoromethyl)pyridin-2-yl)methyl)amino)cyclopentan-1-ol | 261.0 |
| 199 | 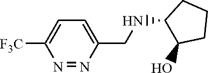 [SMILES: O[C@@H]1CCC[C@H]1NCc1ccc(C(F)(F)F)nn1] | (1R,2R)-2-(((6-(trifluoromethyl)pyridazin-3-yl)methyl)amino)cyclopentan-1-ol | 261.9 |
| 200 | 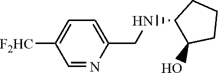 [SMILES: O[C@@H]1CCC[C@H]1NCc1ccc(C(F)F)cn1] | (1R,2R)-2-(((5-(difluoromethyl)pyridin-2-yl)methyl)amino)cyclopentan-1-ol | 243.0 |
| 201 | | 6-((((1R,2R)-2-hydroxycyclopentyl)amino)methyl)nicotinonitrile | 218.0 |
| 202 | | (1R,2R)-2-(((6-bromopyridazin-3-yl)methyl)amino)cyclopentan-1-ol | 272/274 |
| 203 | | (1R,2R)-2-(((6-methoxypyridazin-3-yl)methyl)amino)cyclopentan-1-ol | 224.0 |
| 204 | | (3S,4R)-4-(((5-(trifluoromethyl)pyridin-2-yl)methyl)amino)tetrahydrofuran-3-ol | 262.0 |
| 205 | | (R)-N-((6-bromopyridazin-3-yl)methyl)-1-(3-fluoropyridin-2-yl)ethan-1-amine | 311/313 |
| 206 | | (R)-1-cyclopropyl-N-((5-(trifluoromethyl)pyridin-2-yl)methyl)ethan-1-amine | 245.0 |
| 207 | | (R)-1-(4-fluorophenyl)-N-((5-(trifluoromethyl)pyridin-2-yl)methyl)ethan-1-amine | 299.1 |
| 208 | | (R)-1-(2,4-difluorophenyl)-N-((5-(trifluoromethyl)pyridin-2-yl)methyl)ethan-1-amine | 317.1 |
| 209 | | (R)-1-methoxy-N-((5-(trifluoromethyl)pyridin-2-yl)methyl)propan-2-amine | 249.0 |
| 210 | 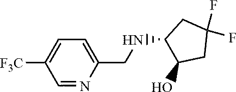 [SMILES: O[C@@H]1CC(F)(F)C[C@H]1NCc1ccc(C(F)(F)F)cn1] | (1R,2R)-4,4-difluoro-2-(((5-(trifluoromethyl)pyridin-2-yl)methyl)amino)cyclopentan-1-ol (racemate) | 297.2 |
| 211 | 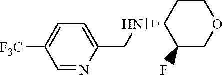 [SMILES: F[C@@H]1COCC[C@H]1NCc1ccc(C(F)(F)F)cn1] | (3S,4R)-3-fluoro-N-((5-(trifluoromethyl)pyridin-2-yl)methyl)tetrahydro-2H-pyran-4-amine (racemate) | 279.2 |
| 212 | 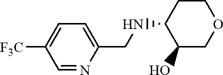 [SMILES: O[C@@H]1COCC[C@H]1NCc1ccc(C(F)(F)F)cn1] | (3S,4R)-4-(((5-(trifluoromethyl)pyridin-2-yl)methyl)amino)tetrahydro-2H-pyran-3-ol (racemate) | 277.2 |
| 213 | 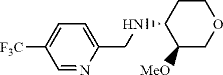 [SMILES: CO[C@@H]1COCC[C@H]1NCc1ccc(C(F)(F)F)cn1] | (3S,4R)-3-methoxy-N-((5-(trifluoromethyl)pyridin-2-yl)methyl)tetrahydro-2H-pyran-4-amine (racemate) | 291.2 |
| 214 | 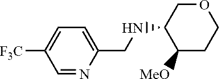 [SMILES: CO[C@@H]1CCOC[C@H]1NCc1ccc(C(F)(F)F)cn1] | (3R,4R)-4-methoxy-N-((5-(trifluoromethyl)pyridin-2-yl)methyl)tetrahydro-2H-pyran-3-amine (racemate) | 291.2 |
| 215 | 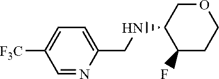 [SMILES: F[C@@H]1CCOC[C@H]1NCc1ccc(C(F)(F)F)cn1] | (3R,4R)-4-fluoro-N-((5-(trifluoromethyl)pyridin-2-yl)methyl)tetrahydro-2H-pyran-3-amine (racemate) | 279.2 |
| 216 | 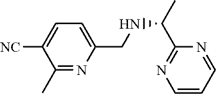 | (R)-2-methyl-6-(((1-(pyrimidin-2-yl)ethyl)amino)methyl)nicotinonitrile | 254.2 |
| 217 | 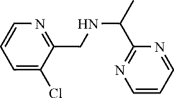 [SMILES: CC(NCc1ncccc1Cl)c1ncccn1] | N-((3-chloropyridin-2-yl)methyl)-1-(pyrimidin-2-yl)ethan-1-amine | 249.0 |
| 218 | 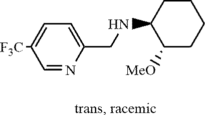 | (1S,2S)-2-methoxy-N-((5-(trifluoromethyl)pyridin-2-yl)methyl)cyclohexan-1-amine | 289.2 |
| 219 | 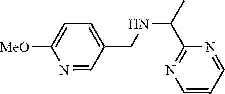 [SMILES: COc1ccc(CNC(C)c2ncccn2)cn1] | N-((6-methoxypyridin-3-yl)methyl)-1-(pyrimidin-2-yl)ethan-1-amine | 245 |
| 245 | | N-(4-(oxetan-3-yl)benzyl)-1-(pyrimidin-2-yl)ethan-1-amine | 270.2 |
| 248 | 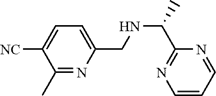 | (R)-2-methyl-6-(((1-(pyrimidin-2-yl)ethyl)amino)methyl)nicotinonitrile | 254.2 |
| 249 | 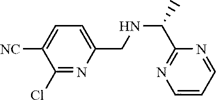 | (R)-2-chloro-6-(((1-(pyrimidin-2-yl)ethyl)amino)methyl)nicotinonitrile | 274.2 |

Intermediate 222: (R)-N-((6-ethoxypyridazin-3-yl)methyl)-1-(pyrimidin-2-yl)ethan-1-amine.

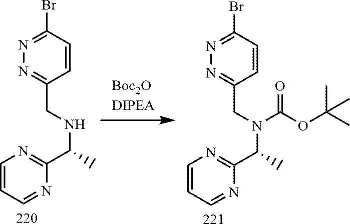

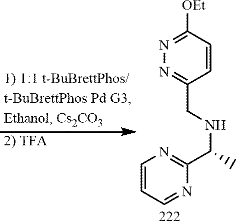

To a solution of 1-(pyrimidin-2-yl)-N-((6-bromopyridizin-3-yl)methyl)propan-1-amine (0.84 g, 3.47 mmol) and DIPEA (0.89 g, 1.212 mL, 6.94 mmol) in DCM (17 mL) at RT was added di-tert-butyl dicarbonate (1.21 g, 5.55 mmol). The reaction mixture was stirred at RT for 18 h then partitioned between DCM and water. The layers were separated. The organic layer was dried over Na₂SO₄ and concentrated. The crude product was purified by silica gel chromatography (0-100% EtOAc in heptane) to give tert-butyl (R)-((6-bromopyridazin-3-yl)methyl)(1-(pyrimidin-2-yl)ethyl)carbamate (1.61 g, 4.70 mmol, 136% yield) as a solid which contained some residual solvents. m/z (ESI): 394.1/396.1 (M+H)⁺.

A solution of t-butylBrettPhos (55 mg, 0.11 mmol, Aldrich) in dioxane (1.0 mL) was added to a mixture of t-butylBrettPhos Pd G3 (98 mg, 0.11 mmol, Aldrich), EtOH (0.33 mL), (R)-((6-bromopyridazin-3-yl)methyl)(1-(pyrimidin-2-yl)ethyl)carbamate (300 mg, 0.76 mmol), and cesium carbonate (350 mg, 1.1 mmol) in dioxane (2.5 mL). The mixture was stirred at RT for 18 h then concentrated under reduced pressure. The residue was purified by silica gel chromatography (0-100% EtOAc in heptane) to give tert-butyl (R)-((6-ethoxypyridazin-3-yl)methyl)(1-(pyrimidin-2-yl)ethyl)carbamate (231 mg, 0.643 mmol, 84% yield) as a brown solid. m/z (ESI): 360.0 (M+H)⁺. This material was treated with TFA (10 mL), stirred for 15 min, then concentrated under reduced pressure. The residue was free based by dissolving in MeOH and eluting through an SCX column with MeOH followed by 2 M ammonia in MeOH to give (R)-N-((6-ethoxypyridazin-3-yl)methyl)-1-(pyrimidin-2-yl)ethan-1-amine (222, 166 mg, 0.64 mmol, 84% yield) as a brown oil. m/z (ESI): 260.0 (M+H)⁺.

Intermediates 224 and 225. (R)-N-((5-ethoxypyridin-2-yl)methyl)-1-(pyrimidin-2-yl)ethan-1-amine (224, m/z (ESI): 259.0 (M+H)⁺) and (R)-N-((5-cyclopropoxypyridin-2-yl)methyl)-1-(pyrimidin-2-yl)ethan-1-amine (225, m/z (ESI): 271.1 (M+H)⁺) were prepared from Intermediate 108 in a manner similar to that described for amine 222.

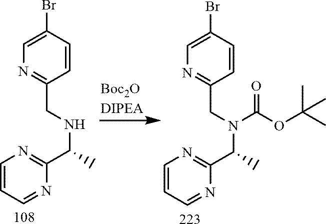

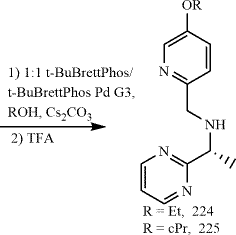

Intermediate 226: (R)-6-(((1-(pyrimidin-2-yl)ethyl)amino)methyl)-N-(2,2,2-trifluoroethyl)pyridazin-3-amine.

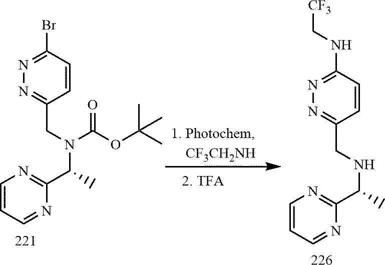

A 4 mL glass vial was charged with 4,4′-di-tert-butyl-2,2′-bipyridine bis(3,5-difluoro-2-(5-(trifluoromethyl)pyridin-2-yl)phenyl)iridium, hexafluorophosphate(V) salt (1 M in DMAc solution) (0.05 mL, 0.050 µmol, Aldrich) (approx. weight), nickel(II) bromide ethylene glycol dimethyl ether complex (5.9 mg, 0.019 mmol, Sigma-Aldrich), 2,2,2-trifluoroethan-1-amine (38 mg, 0.030 mL, 0.38 mmol, Enamine), tert-butyl (R)-((6-bromopyridazin-3-yl)methyl)(1-(pyrimidin-2-yl)ethyl)carbamate (221, 150 mg, 0.38 mmol), DMAc (1 mL), and 1,3,4,6,7,8-hexahydro-1-methyl-2h-pyrimido[1,2-a]pyrimidine (150 mg, 0.14 mL, 0.95 mmol). The vial was sealed and irradiated in the Penn photoreactor (450 nm, 100% LED power, 1500 RPM fan, full stirring) for 18 h. The reaction mixture was diluted with water (15 mL) and extracted with EtOAc (2 × 50 mL). The combined organic layer was washed with water (40 mL) followed by brine (20 mL) and dried over MgSO₄. The crude product was purified by silica gel chromatography (0-100% EtOAc in heptane) to provide tert-butyl (R)-(1-(pyrimidin-2-yl)ethyl)((6-((2,2,2-trifluoroethyl)amino)pyridazin-3-yl)methyl)carbamate (12 mg, 0.028 mmol, 7% yield). This BOC-protected material was dissolved in TFA (5 mL) and stirred for 30 min. The reaction mixture was concentrated, and the residue was eluted through an SCX column with MeOH followed by 2 M ammonia in MeOH to give (R)-6-(((1-(pyrimidin-2-yl)ethyl)amino)methyl)-N-(2,2,2-trifluoroethyl)pyridazin-3-amine (226, 8.6 mg, 0.028 mmol, 7% yield (over two steps)) as a brown oil. m/z (ESI): 313.1 (M+H)⁺.

Intermediate 227: (R)-N-((6-(difluoromethyl)pyridazin-3-yl)methyl)-1-(pyrimidin-2-yl)ethan-1-amine.

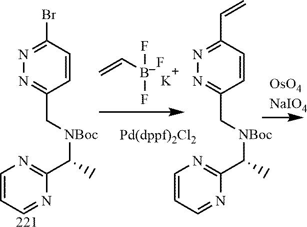

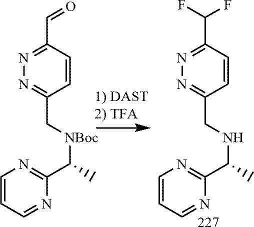

tert-Butyl (R)-((6-bromopyridazin-3-yl)methyl)(1-(pyrimidin-2-yl)ethyl)carbamate (221, 0.20 g, 0.51 mmol), potassium trifluoro(vinyl)borate (0.14 g, 1.0 mmol, Oakwood Products), 1,1′-bis(diphenylphosphino)ferrocene palladium(II)dichloride dichloromethane adduct (10 mg, 0.013 mmol, Strem Chemicals), K₂CO₃ (0.280 g, 2.0 mmol), 1,4-dioxane (1.9 mL) and water (0.63 mL) were added to a 20 mL glass vial. The vial was evacuated and backfilled with nitrogen 3x then sealed and heated at 80° C. After 2 h ~60% conversion was observed. Additional vinyl borate was added (~50 mg) and the reaction was stirred at 80° C. for 2 h. Purification of the reaction mixture via silica gel column (10-100% EtOAc in heptane) provided tert-butyl (R)-(1-(pyrimidin-2-yl)ethyl)((6-vinylpyridazin-3-yl)methyl)carbamate (0.11 g, 0.33 mmol, 65% yield) as a white solid. m/z (ESI): 342.0 (M+H)⁺.

To a mixture of tert-butyl (R)-(1-(pyrimidin-2-yl)ethyl)((6-vinylpyridazin-3-yl)methyl)carbamate (110 mg, 0.33 mmol) in 4:1 THF-H₂O (1.7 mL) was added osmium(VIII) oxide (110 mg, 100 µL, 0.017 mmol, Sigma-Aldrich) followed by sodium periodate (210 mg, 0.99 mmol, Sigma-Aldrich). The resulting reddish-brown slurry was stirred at RT for seven days to near completion (~85%). The reaction mixture was then diluted with EtOAc (30 mL) and washed with saturated sodium bicarbonate (20 mL). The layers were separated and the organic layer was washed again with saturated aqueous sodium bicarbonate (20 mL) followed by brine (15 mL) and dried over MgSO₄. The filtrate was concentrated to give tert-butyl (R)-((6-formylpyridazin-3-yl)methyl)(1-(pyrimidin-2-yl)ethyl)carbamate (70 mg, 0.20 mmol, 62% yield) (~60% purity) as a brown oil that was used directly in the next reaction. m/z (ESI): 344.1 (M+H)⁺.

To a solution of tert-butyl (R)-((6-formylpyridazin-3-yl)methyl)(1-(pyrimidin-2-yl)ethyl)carbamate (70 mg, 0.20 mmol) in DCM (2.0 mL) at 0° C. was added DAST (160 mg, 1.0 mmol, Aldrich). The reaction mixture was slowly warmed to RT over 1.5 h then quenched by dropwise addition of saturated sodium bicarbonate (15 mL). This mixture was then extracted with DCM (2 × 30 mL). The combined organic layer was then dried over MgSO₄ and concentrated. The crude product was purified by silica gel chromatography (0-100% EtOAc in heptane) to give tert-butyl (R)-((6-(difluoromethyl)pyridazin-3-yl)methyl)(1-(pyrimidin-2-yl)ethyl)carbamate (11.1 mg). This material was treated with 3.0 mL of TFA and stirred for 45 min. The reaction mixture was concentrated and the residue was dissolved in MeOH and eluted through an SCX column with MeOH followed by 2 M ammonia in MeOH to give (R)-N-((6-(difluoromethyl)pyridazin-3-yl)methyl)-1-(pyrimidin-2-yl)ethan-1-amine (227, 9.2 mg, 0.035 mmol, 17% yield (over three steps)). m/z (ESI): 266.1 (M+H)⁺.

Intermediates 228 and 229. (R)-N-((6-(3,3-Difluoropyrrolidin-1-yl)pyridazin-3-yl)methyl)-1-(pyrimidin-2-yl)ethan-1-amine (228, m/z (ESI): 321.0 (M+H)⁺) and (R)-N-((6-(3,3-difluoroazeridin-1-yl)pyridazin-3-yl)methyl)-1-(pyrimidin-2-yl)ethan-1-amine (229, m/z (ESI): 307.0 (M+H)⁺) were prepared from Intermediate 222 in a manner similar to that described for amine 226.

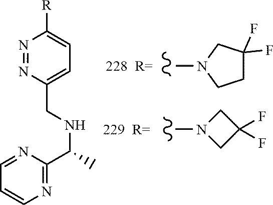

Intermediate 230. (R)-6-(((1-(3-fluoropyridin-2-yl)ethyl)amino)methyl)pyridazine-3-carbonitrile (230, m/z (ESI): 258.0 (M+H)⁺) was prepared from Intermediate 205 in a manner similar to that described for Example 549.

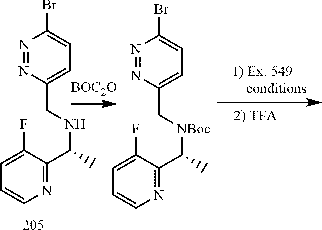

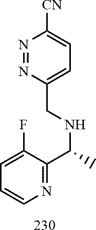

Intermediate 231. (R)-1-(3-fluoropyridin-2-yl)-N-((6-morpholinopyridazin-3-yl)methyl)ethan-1-amine (231, m/z (ESI): 318.0 (M+H)⁺) was prepared from Intermediate 205 in a manner similar to that described for Example 554.

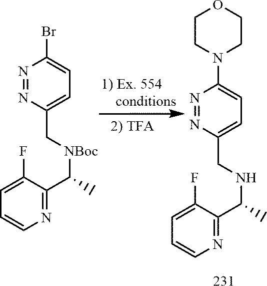

Intermediates 234: (R)-N-((6-bromopyridazin-3-yl)methyl)-5,6,7,8-tetrahydro-[1,2,4]triazolo[1,5-a]pyridin-8-amine. Racemic amine 232 was prepared in a fashion similar to that described above for amine 115a. m/z (ESI): 309/311 (M+H)⁺. The racemic amine was subjected to chiral SFC to provide enantiomerically pure amines 233 (> 90% ee) and 234 (> 96% ee). SFC conditions : Chiral Technologies AS column (250 × 21 mm, 5 µm) with a mobile phase of 80% liquid CO₂ and 20% EtOH with 0.2% TEA using a flowrate of 80 mL/min.

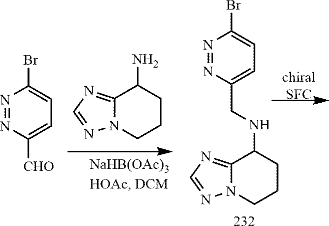

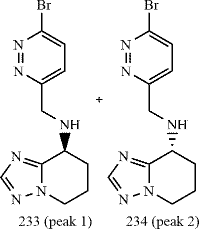

Intermediate 236: (R)-N-((6-(3,6-dihydro-2H-pyran-4-yl)pyridazin-3-yl)methyl)-1-(pyrimidin-2-yl)ethan-1-amine hydrochloride.

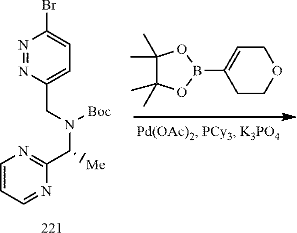

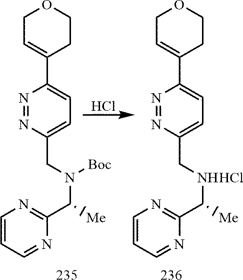

A scintillation vial was charged with 2-(3,6-dihydro-2H-pyran-4-yl)-4,4,5,5-tetramethyl-1,3,2-dioxaborolane (120 mg, 0.571 mmol, Combi Blocks), tert-butyl (R)-((6-bromopyridazin-3-yl)methyl)(1-(pyrimidin-2-yl)ethyl)carbamate (221, 150 mg, 0.380 mmol), potassium phosphate tribasic (242 mg, 1.141 mmol), palladium acetate (12.81 mg, 0.057 mmol), tricyclohexylphosphine (32.0 mg, 0.114 mmol), toluene (3.4 mL), and H₂O (0.38 mL). The mixture was purged with nitrogen for 10 min then heated at 90° C. for 12 h. The reaction mixture was cooled to RT and concentrated under reduced pressure. The crude residue was purified by column chromatography over silica gel (0%-100% (EtOAc:EtOH, 3:1) in heptane to give tert-butyl (R)-((6-(3,6-dihydro-2H-pyran-4-yl)pyridazin-3-yl)methyl)(1-(pyrimidin-2-yl)ethyl)carbamate (235, 130 mg, 0.32 mmol, 86% yield) as a white solid. m/z (ESI)— 398.0 (M+H)⁺. The white solid (130 mg, 0.327 mmol) was suspended in DCM (3.3 mL) and treated with HC1 (0.050 mL of 4 M in dioxane). The mixture was stirred at RT for 12 h then concentrated under reduced pressure to provide the title compound (236) as a brown solid which was used in the subsequent step without further purification. m/z (ESI): 298.1 (M+H)⁺.

Intermediate 238: (R)-N-((5-(oxetan-3-yloxy)pyridin-2-yl)methyl)-1-(pyrimidin-2-yl)ethan-1-amine.

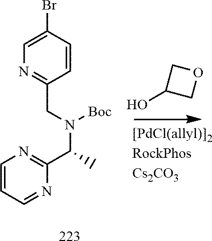

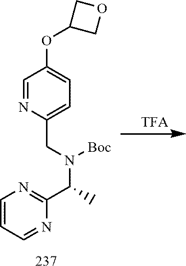

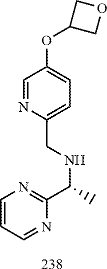

A scintillation vial was charged with Cs₂CO₃ (1.243 g, 3.81 mmol), allylpalladium(ii) chloride dimer (0.093 g, 0.254 mmol), 2-(di-t-butylphosphono)-3-methoxy-6-methyl-2′-4′-6′-tri-i-propyl-1,1′-biphenyl, (0.143 g, 0.305 mmol), oxetan-3-ol (0.471 g, 6.36 mmol), and toluene (5.09 mL). The mixture was purged with N₂ for 10 min, heated at 90° C. for 3 min, then treated with tert-butyl (R)-((5-bromopyridin-2-yl)methyl)(1-(pyrimidin-2-yl)ethyl)carbamate (223, 1.00 g, 2.54 mmol). The mixture was stirred at 90° C. for 18 h, then cooled to RT and concentrated under reduced pressure. The crude residue was purified by column chromatography over silica gel (0-100% (3:1 EtOAc:EtOH) in heptane) to give tert-butyl (R)-((5-(oxetan-3-yloxy)pyridin-2-yl)methyl)(1-(pyrimidin-2-yl)ethyl)carbamate (237, 1.00 g, 2.59 mmol, 100% yield) as a yellow oil. m/z (ESI): 387.2 (M+H)⁺. The yellow oil in DCM (25.9 mL) was treated with TFA (4 mL) and stirred at RT for 12 h. The reaction mixture was concentrated under reduced pressure and the residue was purified by column chromatography over silica gel (0-100% (89:9:1 = DCM:MeOH:NH₃) in heptane) to give (R)-N-((5-(oxetan-3-yloxy)pyridin-2-yl)methyl)-1-(pyrimidin-2-y1)ethan-1-amine (238, 677 mg, 2.36 mmol, 91% yield) as a white solid. m/z (ESI): 287.2 (M+H)⁺.

Intermediate 239: (1R,2R)-2-methoxy-N-((5-(trifluoromethy1)pyridin-2-yl)methyl)-2,3-dihydro-1 H-inden-1 -amine.

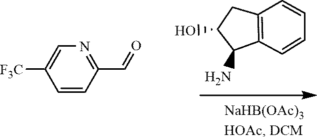

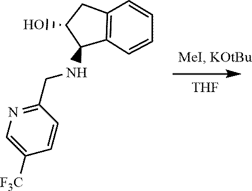

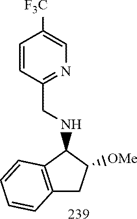

To a 25-mL scintillation vial was added 5-(trifluoromethyl)picolinaldehyde (200 mg, 1.142 mmol), (1R,2R)-1-amino-2,3-dihydro-1H-inden-2-ol (170 mg, 1.142 mmol, Sigma-Aldrich), triethylamine (289 mg, 0.398 mL, 2.86 mmol) and DCM (11.4 mL). The mixture was let stir at rt for 10 min, and then acetic acid (82 mg, 0.078 mL, 1.371 mmol) was added. Mixture was allowed to stir for 10 min, then sodium triacetoxyborohydride (363 mg, 1.713 mmol) was added. The mixture was allowed to stir at rt overnight. Upon completion, the reaction was concentrated and directly loaded onto a redisep cartridge for chromatography with 0-100% (89:9:1 DCM:MeOH:NH₃) in heptanes to give (1R,2R)-1-(((5-(trifluoromethyl)pyridin-2-yl)methyl)amino)-2,3-dihydro-1H-inden-2-ol (298 mg, 0.967 mmol, 85% yield). m/z (ESI): 309.2 (M+H)⁺.

To a 0° C. solution of (1R,2R)-1-(((5-(trifluoromethyl)pyridin-2-yl)methyl)amino)-2,3-dihydro-1H-inden-2-ol (298 mg, 0.967 mmol) and THF (10 mL) was added potassium t-butoxide (130 mg, 1.160 mmol) in one portion. The reaction was stirred for 15 min, then treated with methyl iodide (206 mg, 0.090 mL, 1.45 mmol) dropwise. The reaction was allowed to stir overnight. Upon completion, the reaction was concentrated and purified by silica gel chromatography with 0-40% (3:1 EtOAc:EtOH) in heptane to give intermediate 239 (37 mg, 0.114 mmol, 12% yield). m/z (ESI): 323.2 (M+H)⁺.

Intermediate 240: (1S,2S)-2-methoxy-N-((5-(trifluoromethyl)pyridin-2-yl)methyl)-2,3-dihydro-1 H-inden-1 -amine.

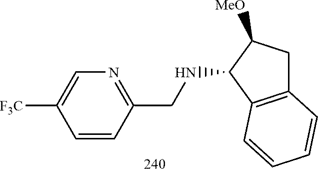

(1S,2S)methoxy-N-((5-(trifluoromethyl)pyridinyl)methy)-2,3-dihydro-1H-inden-1-amine was prepared in a similar to 239. m/z (ESI): 323.2 (M+H)⁺.

Intermediate 242: (R)-1-(6-(((1-(pyrimidin-2-yl)ethyl)amino)methyl)pyridin-3-yl)ethan-1-one hydrochloride.

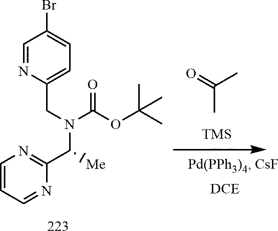

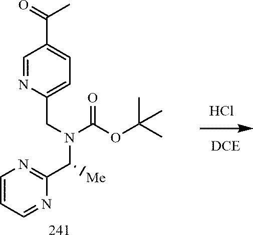

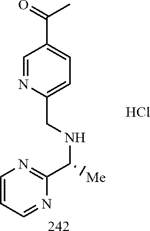

To a 25-mL round-bottomed flask was added tert-butyl (R)-((5-bromopyridin-2-yl)methyl)(1-(pyrimidin-2-yl)ethyl)carbamate (223, 200 mg, 0.509 mmol), Pd(PPh₃)₄ (58.8 mg, 0.051 mmol), and cesium fluoride (309 mg, 2.034 mmol). The vial was evacuated and backfilled with nitrogen 3x. Then, DCE (509 µL) and 1-(trimethylsilyl)ethan-1-one (118 mg, 118 µL, 1.017 mmol, Sigma-Aldrich) were added. The reaction was purged with nitrogen for 10 min and placed in a heating block kept at 75° C. for 8 h. Upon completion, the mixture was directly loaded onto a redisep cartridge for chromatography. Compound 241 eluted with 0-50% (89:9:1 DCM:MeOH:NH3) in heptane as a yellow oil. m/z (ESI): 357.2 (M+H)⁺.

To a 25-mL vial was added tert-butyl (R)-((5-acetylpyridin-2-yl)methyl)(1-(pyrimidin-2-yl)ethyl)carbamate (241, 50 mg, 0.140 mmol), DCM (1.4 mL), and hydrochloric acid (0.015 mL, 0.421 mmol). The reaction was allowed to stir at rt overnight. Upon completion, the reaction was concentrated and used in the next step as is assuming quantitative yield. m/z (ESI): 257.2 (M+H)⁺.

Intermediate 244: (R)-2-(6-(((1-(pyrimidin-2-yl)ethyl)amino)methyl)pyridin-3-yl)propan-2-ol.

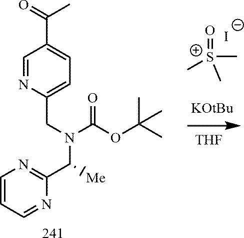

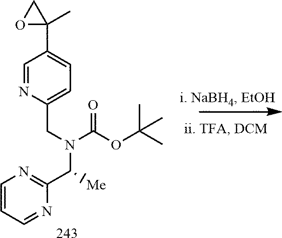

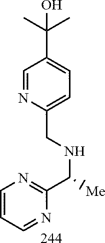

To a 25-mL scintillation vial was added trimethylsulfoxonium iodide (309 mg, 1.403 mmol), potassium tert-butoxide (157 mg, 1.403 mmol) and THF (3.5 mL). The mixture was allowed to stir under nitrogen at 50° C. for 30 min. Then, tert-butyl (R)-((5-acetylpyridin-2-yl)methyl)(1-(pyrimidin-2-yl)ethyl)carbamate (241, 125 mg, 0.351 mmol) was added and the mixture was allowed to stir at 50° C. overnight. Upon completion, the mixture was directly loaded onto a redisep cartridge for chromatography with 0-50% (89:9:1 DCM:MeOH:NH3) in heptane to give 243 (57 mg, 0.154 mmol, 44% yield) as a yellow oil. m/z (ESI): 371.2 (M+H)′.

To a 25-mL scintillation vial was added tert-butyl ((5-(2-methyloxiran-2-yl)pyridin-2-yl)methyl)((R)-1-(pyrimidin-2-yl)ethyl)carbamate (243, 57 mg, 0.154 mmol) and EtOH (1.5 mL). The mixture was cooled to 0° C. and NaBH₄ (14.55 mg, 0.385 mmol) was added in 1 portion. The ice bath was removed and reaction was allowed to warm to rt overnight. Upon completion, the reaction was concentrated to dryness and redissolved in 1 mL of DCM (1 mL) and TFA (1754 mg, 1.185 mL, 15.39 mmol). The reaction was allowed to stir at rt for 2 h. Upon Boc-group removal, the reaction was concentrated and used in the following step as is assuming quantitative yield. m/z (ESI): 273.3 (M+H)⁺.

Intermediate 246: (R)-N-((6-cyclopropylpyridazin-3-yl)methyl)-1-(pyrimidin-2-yl)ethan-1-amine.

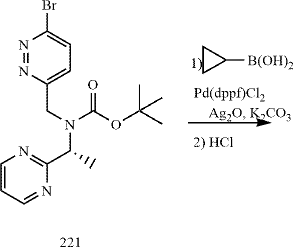

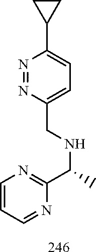

A mixture of tert-butyl (R)-((6-bromopyridazin-3-yl)methyl)(1-(pyrimidin-2-yl)ethyl)carbamate (0.2 g, 0.507 mmol, 221), cyclopropylboronic acid (0.218 g, 2.54 mmol, Combi-Blocks), [1,1′-bis(diphenylphosphino)ferrocene]-dichloropalladium (ii) dichloromethane complex (0.041 g, 0.051 mmol, Oakwood Products, Inc.), silver(i) oxide (0.223 g, 0.964 mmol, Sigma-Aldrich Corporation), potassium carbonate (0.210 g, 1.522 mmol, Acros) and 1,4-dioxane (5 mL) was purged with Ar, then stirred in a sealed vial at 80° C. for 4.5 h. LCMS showed the desired product. The mixture was filtered through celite and concentrated in vacuo. The crude material was purified by chromatography through a silica gel column, eluting with 0-100% (3/1 EtOAc/EtOH) in heptane. The product was obtained as off-white solid (0.149 g, 83%). MS: m/z(ESI): 356.3 (M+H)+.

To a solution of tert-butyl (R)-((6-cyclopropylpyridazin-3-yl)methyl)(1-(pyrimidin-2-yl)ethyl)carbamate (0.14 g, 0.394 mmol) in dichloromethane (4 mL) was added HC1, 4.0 M in dioxane (0.788 mL, 3.15 mmol, Aldrich). The solution became a suspension. MeOH was added to make the suspension to a solution again. The mixture was stirred at rt overnight. LCMS showed the product. The mixture was concentrated in vacuo. The product was obtained as orange solid. MS: m/z (ESI): 256 (M+H)+.

Intermediate 247: (R)-N-((5-morpholinopyridin-2-yl)methyl)-1-(pyrimidin-2-yl)ethan-1-amine.

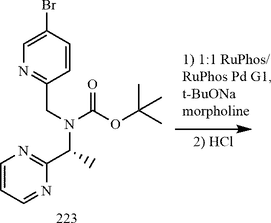

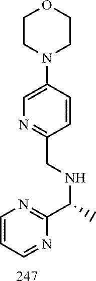

A mixture of tert-butyl (R)-((5-bromopyridin-2-yl)methyl)(1-(pyrimidin-2-yl)ethy)carbamate (0.350 g, 0.890 mmol, 223), morpholine (0.233 g, 0.234 mL, 2.67 mmol, Aldrich), 2-methyltetrahydrofuran (5.5 mL), dicyclohexy(2′,6′-diisopropoxy-[1,1′-biphenyl]-2-yl)phosphane (RuPhos) (0.145 g, 0.311 mmol, Aldrich), RuPhos Pd G1 methyl t-butyl ether adduct (0.254 g, 0.311 mmol, Strem), and sodium tert-butoxide (0.257 g, 2.67 mmol, Aldrich) in a vial was purged with Ar for 5 min, then the mixture was heated at 80° C. overnight. LCMS showed the product. The reaction mixture was filtered over a pad of diatomaceous earth and washed with EtOAc. The filtrate was concentrated in vacuo and the residue was purified by silica gel chromatography: 0-100% EtOAc/EtOH (3/1) in DCM. The product was obtained as orange solid (0.163 g, 46%). MS: m/z (ESI): 400 (M+H)+.

To a solution of tert-butyl (R)-((5-morpholinopyridin-2-yl)methyl)(1-(pyrimidin-2-yl)ethyl)carbamate (0.163 g, 0.408 mmol) in dichloromethane (4 mL) was added HC1, 4.0 M in dioxane (0.918 mL, 3.67 mmol, Aldrich). The solution became a suspension. MeOH was added to make the suspension to a solution. The mixture was stirred at rt overnight. LCMS showed the product. The mixture was concentrated in vacuo. The product was obtained as orange solid. MS: m/z (ESI): 300 (M+H) ⁺.

### Examples

Example 300: 2-amino-N-isobutyl-3-methyl-N-((5-(trifluoromethyl)pyridin-2-yl)methyl)quinoline-6-carboxamide. (Method A-HATU)

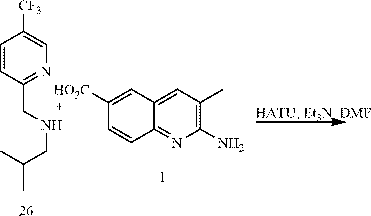

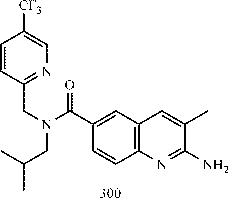

HATU (217 mg, 0.57 mmol, ChemPep) was added to a mixture of 2-amino-3-methylquinoline-6-carboxylic acid (1, 105 mg, 0.52 mmol), 2-methyl-N-((5-(trifluoromethyl)pyridin-2-yl)methyl)propan-1-amine (26, 133 mg, 0.57 mmol) and triethylamine (0.15 mL, 1.04 mmol) in 2 mL of DMF at RT. The mixture was stirred at RT for 4 h then partitioned between 50 mL of EtOAc and 5 mL of water. The layers were separated. The organic layer was washed with 5 mL of 1 N NaOH and concentrated. The residue was purified on a silica gel column (20-90% EtOAc in heptane) to give the title compound (300, 177 mg, 0.43 mmol, 82% yield) as a light-yellow solid. m/z (ESI): 417.0 (M+H)⁺. ¹H NMR (CHLOROFORM-d, 400 MHz) δ 8.84 (br s, 1H), 7.92 (br s, 1H), 7.5-7.8 (m, 5H), 5.20 (br s, 2H), 4.94 (m, 1H), 4.75 (m, 1H), 3.31 (m, 2H), 2.31 (s, 3H), 2.04 (m, 1H), 0.58-1.10 (m, 6H).

TABLE 8

Examples 301 to 344 were prepared in a manner similar to that described above (Method A-HATU) for Example 300

| Ex. # | Chemical Structure | Name | LRMS: (ESI + ve ion) m/z |
|---|---|---|---|
| 301 | 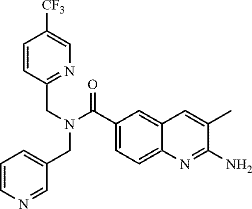 [SMILES: Cc1cc2cc(C(=O)N(Cc3cccnc3)Cc3ccc(C(F)(F)F)cn3)ccc2nc1N] | 2-amino-3-methyl-N-(3-pyridinylmethyl)-N-((5-(trifluoromethyl)-2-pyridinyl)methyl)-6-quinolinecarboxamide | 452.1 |
| 302 | 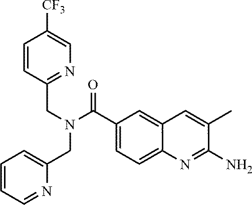 [SMILES: Cc1cc2cc(C(=O)N(Cc3ccccn3)Cc3ccc(C(F)(F)F)cn3)ccc2nc1N] | 2-amino-3-methyl-N-(2-pyridinylmethyl)-N-((5-(trifluoromethyl)-2-pyridinyl)methyl)-6-quinolinecarboxamide | 452.1 |
| 303 | 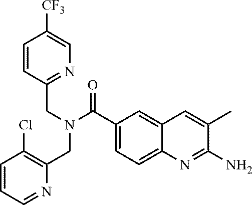 [SMILES: Cc1cc2cc(C(=O)N(Cc3ccc(C(F)(F)F)cn3)Cc3ncccc3Cl)ccc2nc1N] | 2-amino-N-((3-chloro-2-pyridinyl)methyl)-3-methyl-N-((5-(trifluoromethyl)-2-pyridinyl)methyl)-6-quinolinecarboxamide | 486 |
| 304 | 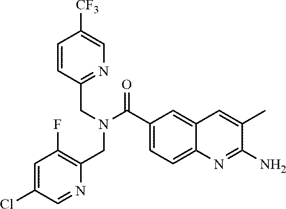 [SMILES: Cc1cc2cc(C(=O)N(Cc3ccc(C(F)(F)F)cn3)Cc3ncc(Cl)cc3F)ccc2nc1N] | 2-amino-N-((5-chloro-3-fluoro-2-pyridinyl)methyl)-3-methyl-N-((5-(trifluoromethyl)-2-pyridinyl)methyl)-6-quinolinecarboxamide | 504 |
| 305 | 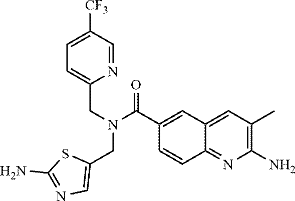 [SMILES: Cc1cc2cc(C(=O)N(Cc3ccc(C(F)(F)F)cn3)Cc3cnc(N)s3)ccc2nc1N] | 2-amino-N-((2-amino-1,3-thiazol-5-yl)methyl)-3-methyl-N-((5-(trifluoromethyl)-2-pyridinyl)methyl)-6-quinolinecarboxamide | 472.5 |
| 306 | 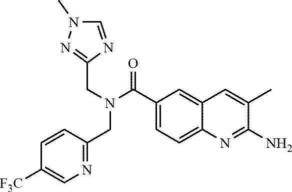 [SMILES: Cc1cc2cc(C(=O)N(Cc3ccc(C(F)(F)F)cn3)Cc3ncn(C)n3)ccc2nc1N] | 2-amino-3-methyl-N-((1-methyl-1H-1,2,4-triazol-3-yl)methyl)-N-((5-(trifluoromethyl)-2-pyridinyl)methyl)-6-quinolinecarboxamide | 456.1 |
| 307 | 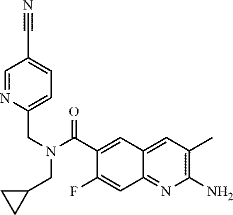 [SMILES: Cc1cc2cc(C(=O)N(Cc3ccc(C#N)cn3)CC3CC3)c(F)cc2nc1N] | 2-amino-N-((5-cyano-2-pyridinyl)methyl)-N-(cyclopropylmethyl)-7-fluoro-3-methyl-6-quinolinecarboxamide | 390 |
| 308 | 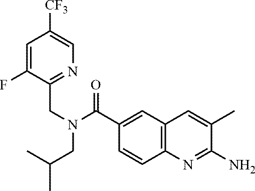 [SMILES: Cc1cc2cc(C(=O)N(Cc3ncc(C(F)(F)F)cc3F)CC(C)C)ccc2nc1N] | 2-amino-N-((3-fluoro-5-(trifluoromethyl)-2-pyridinyl)methyl)-3-methyl-N-(2-methylpropyl)-6-quinolinecarboxamide | 435.2 |
| 309 | 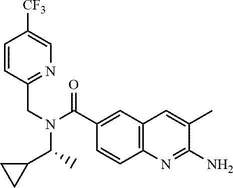 [SMILES: Cc1cc2cc(C(=O)N(Cc3ccc(C(F)(F)F)cn3)[C@H](C)C3CC3)ccc2nc1N] | 2-amino-N-((1R)-1-cyclopropylethyl)-3-methyl-N-((5-(trifluoromethyl)-2-pyridinyl)methyl)-6-quinolinecarboxamide | 429.2 |
| 310 | 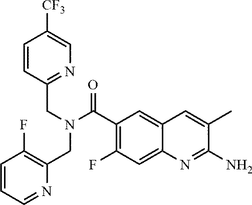 [SMILES: Cc1cc2cc(C(=O)N(Cc3ccc(C(F)(F)F)cn3)Cc3ncccc3F)c(F)cc2nc1N] | 2-amino-7-fluoro-N-((3-fluoro-2-pyridinyl)methyl)-3-methyl-N-((5-(trifluoromethyl)-2-pyridinyl)methyl)-6-quinolinecarboxamide | 488 |
| 311 | 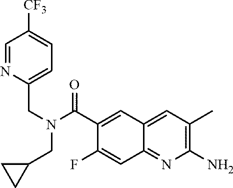 [SMILES: Cc1cc2cc(C(=O)N(Cc3ccc(C(F)(F)F)cn3)CC3CC3)c(F)cc2nc1N] | 2-amino-N-(cyclopropylmethyl)-7-fluoro-3-methyl-N-((5-(trifluoromethyl)-2-pyridinyl)methyl)-6-quinolinecarboxamide | 433.3 |
| 312 | 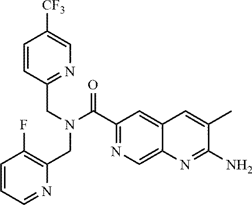 [SMILES: Cc1cc2cc(C(=O)N(Cc3ccc(C(F)(F)F)cn3)Cc3ncccc3F)ncc2nc1N] | 2-amino-N-((3-fluoro-2-pyridinyl)methyl)-3-methyl-N-((5-(trifluoromethyl)-2-pyridinyl)methyl)-1,7-naphthyridine-6-carboxamide | 471 |
| 313 | 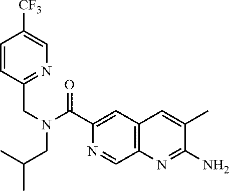 [SMILES: Cc1cc2cc(C(=O)N(Cc3ccc(C(F)(F)F)cn3)CC(C)C)ncc2nc1N] | 2-amino-3-methyl-N-(2-methylpropyl)-N-((5-(trifluoromethyl)-2-pyridinyl)methyl)-1,7-naphthyridine-6-carboxamide | 418.1 |
| 314 | 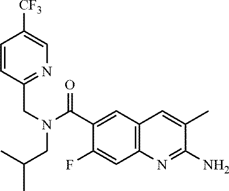 [SMILES: Cc1cc2cc(C(=O)N(Cc3ccc(C(F)(F)F)cn3)CC(C)C)c(F)cc2nc1N] | 2-amino-7-fluoro-3-methyl-N-(2-methylpropyl)-N-((5-(trifluoromethyl)-2-pyridinyl)methyl)-6-quinolinecarboxamide | 435.2 |
| 315 | 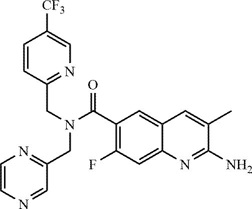 [SMILES: Cc1cc2cc(C(=O)N(Cc3ccc(C(F)(F)F)cn3)Cc3cnccn3)c(F)cc2nc1N] | 2-amino-7-fluoro-3-methyl-N-(2-pyrazinylmethyl)-N-((5-(trifluoromethyl)-2-pyridinyl)methyl)-6-quinolinecarboxamide | 471.2 |
| 316 | 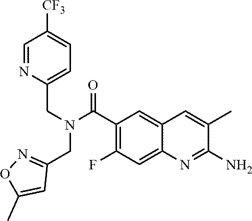 [SMILES: Cc1cc(CN(Cc2ccc(C(F)(F)F)cn2)C(=O)c2cc3cc(C)c(N)nc3cc2F)no1] | 2-amino-7-fluoro-3-methyl-N-((5-methyl-1,2-oxazol-3-yl)methyl)-N-((5-(trifluoromethyl)-2-pyridinyl)methyl)-6-quinolinecarboxamide | 474.2 |
| 317 | 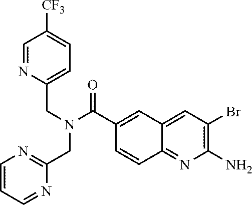 [SMILES: Nc1nc2ccc(C(=O)N(Cc3ccc(C(F)(F)F)cn3)Cc3ncccn3)cc2cc1Br] | 2-amino-3-bromo-N-(2-pyrimidinylmethyl)-N-((5-(trifluoromethyl)-2-pyridinyl)methyl)-6-quinolinecarboxamide | 517.0/519.0 |
| 318 | 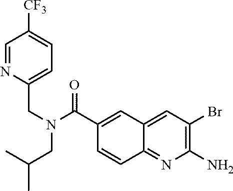 [SMILES: CC(C)CN(Cc1ccc(C(F)(F)F)cn1)C(=O)c1ccc2nc(N)c(Br)cc2c1] | 2-amino-3-bromo-N-(2-methylpropyl)-N-((5-(trifluoromethyl)-2-pyridinyl)methyl)-6-quinolinecarboxamide | 481.1/483.0 |
| 319 | 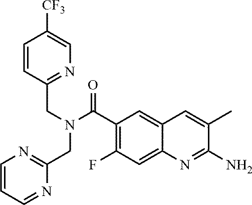 [SMILES: Cc1cc2cc(C(=O)N(Cc3ccc(C(F)(F)F)cn3)Cc3ncccn3)c(F)cc2nc1N] | 2-amino-7-fluoro-3-methyl-N-(2-pyrimidinylmethyl)-N-((5-(trifluoromethyl)-2-pyridinyl)methyl)-6-quinolinecarboxamide | 471 |
| 320 | 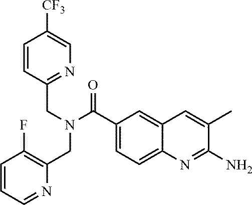 [SMILES: Cc1cc2cc(C(=O)N(Cc3ccc(C(F)(F)F)cn3)Cc3ncccc3F)ccc2nc1N] | 2-amino-N-((3-fluoro-2-pyridinyl)methyl)-3-methyl-N-((5-(trifluoromethyl)-2-pyridinyl)methyl)-6-quinolinecarboxamide | 470.1 |
| 321 | 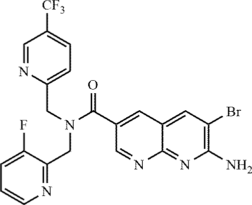 [SMILES: Nc1nc2ncc(C(=O)N(Cc3ccc(C(F)(F)F)cn3)Cc3ncccc3F)cc2cc1Br] | 7-amino-6-bromo-N-((3-fluoro-2-pyridinyl)methyl)-N-((5-(trifluoromethyl)-2-pyridinyl)methyl)-1,8-naphthyridine-3-carboxamide | 535/537 |
| 322 | 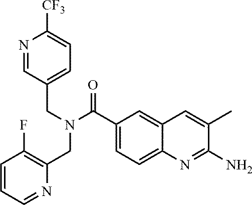 [SMILES: Cc1cc2cc(C(=O)N(Cc3ccc(C(F)(F)F)nc3)Cc3ncccc3F)ccc2nc1N] | 2-amino-N-((3-fluoro-2-pyridinyl)methyl)-3-methyl-N-((6-(trifluoromethyl)-3-pyridinyl)methyl)-6-quinolinecarboxamide | 470.1 |
| 323 | 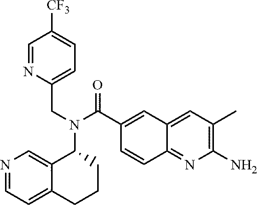 [SMILES: Cc1cc2cc(C(=O)N(Cc3ccc(C(F)(F)F)cn3)[C@@H]3CCCc4ccncc43)ccc2nc1N] | 2-amino-3-methyl-N-((8R)-5,6,7,8-tetrahydro-8-isoquinolinyl)-N-((5-(trifluoromethyl)-2-pyridinyl)methyl)-6-quinolinecarboxamide | 492.2 |
| 324 | 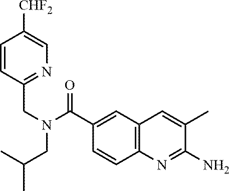 [SMILES: Cc1cc2cc(C(=O)N(Cc3ccc(C(F)F)cn3)CC(C)C)ccc2nc1N] | 2-amino-N-((5-(difluoromethyl)-2-pyridinyl)methyl)-3-methyl-N-(2-methylpropyl)-6-quinolinecarboxamide | 399.2 |
| 325 | 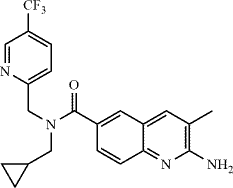 [SMILES: Cc1cc2cc(C(=O)N(Cc3ccc(C(F)(F)F)cn3)CC3CC3)ccc2nc1N] | 2-amino-N-(cyclopropylmethyl)-3-methyl-N-((5-(trifluoromethyl)-2-pyridinyl)methyl)-6-quinolinecarboxamide | 415 |
| 326 | 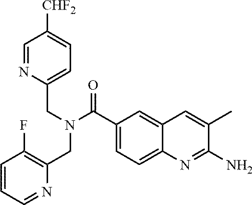 [SMILES: Cc1cc2cc(C(=O)N(Cc3ccc(C(F)F)cn3)Cc3ncccc3F)ccc2nc1N] | 2-amino-N-((5-(difluoromethyl)-2-pyridinyl)methyl)-N-((3-fluoro-2-pyridinyl)methyl)-3-methyl-6-quinolinecarboxamide | 452 |
| 327 | 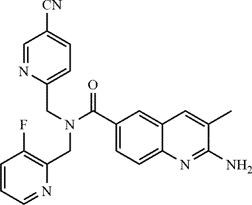 [SMILES: Cc1cc2cc(C(=O)N(Cc3ccc(C#N)cn3)Cc3ncccc3F)ccc2nc1N] | 2-amino-N-((5-cyano-2-pyridinyl)methyl)-N-((3-fluoro-2-pyridinyl)methyl)-3-methyl-6-quinolinecarboxamide | 427 |
| 328 | 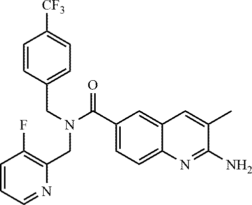 [SMILES: Cc1cc2cc(C(=O)N(Cc3ccc(C(F)(F)F)cc3)Cc3ncccc3F)ccc2nc1N] | 2-amino-N-((3-fluoro-2-pyridinyl)methyl)-3-methyl-N-(4-(trifluoromethyl)benzyl)-6-quinolinecarboxamide | 469 |
| 329 | 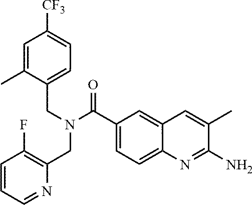 [SMILES: Cc1cc(C(F)(F)F)ccc1CN(Cc1ncccc1F)C(=O)c1ccc2nc(N)c(C)cc2c1] | 2-amino-N-((3-fluoro-2-pyridinyl)methyl)-3-methyl-N-(2-methyl-4-(trifluoromethyl)benzyl)-6-quinolinecarboxamide | 483 |
| 330 | 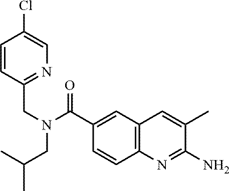 [SMILES: Cc1cc2cc(C(=O)N(Cc3ccc(Cl)cn3)CC(C)C)ccc2nc1N] | 2-amino-N-((5-chloro-2-pyridinyl)methyl)-3-methyl-N-(2-methylpropyl)-6-quinolinecarboxamide | 383 |
| 331 | 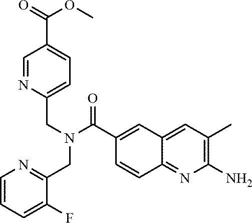 [SMILES: COC(=O)c1ccc(CN(Cc2ncccc2F)C(=O)c2ccc3nc(N)c(C)cc3c2)nc1] | methyl 6-((((2-amino-3-methyl-6-quinolinyl)carbonyl)((3-fluoro-2-pyridinyl)methyl)amino)methyl)-3-pyridinecarboxylate | 460.1 |
| 332 | 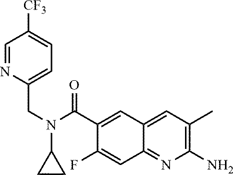 [SMILES: Cc1cc2cc(C(=O)N(Cc3ccc(C(F)(F)F)cn3)C3CC3)c(F)cc2nc1N] | 2-amino-N-cyclopropyl-7-fluoro-3-methyl-N-((5-(trifluoromethyl)-2-pyridinyl)methyl)-6-quinolinecarboxamide | 419 |
| 333 | 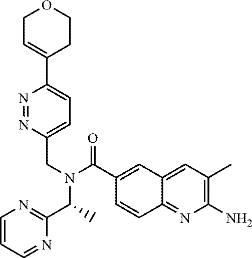 [SMILES: Cc1cc2cc(C(=O)N(Cc3ccc(C4=CCOCC4)nn3)[C@H](C)c3ncccn3)ccc2nc1N] | 2-amino-N-((6-(3,6-dihydro-2H-pyran-4-yl)-3-pyridazinyl)methyl)-3-methyl-N-((1R)-1-(2-pyrimidinyl)ethyl)-6-quinolinecarboxamide | 482 |
| 334 | 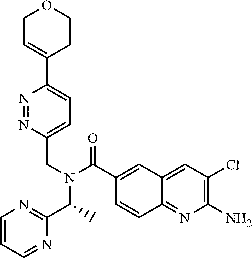 [SMILES: C[C@H](c1ncccn1)N(Cc1ccc(C2=CCOCC2)nn1)C(=O)c1ccc2nc(N)c(Cl)cc2c1] | 2-amino-3-chloro-N-((6-(3,6-dihydro-2H-pyran-4-yl)-3-pyridazinyl)methyl)-N-((1R)-1-(2-pyrimidinyl)ethyl)-6-quinolinecarboxamide | 502 |
| 335 | 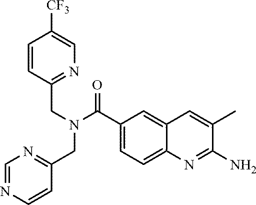 [SMILES: Cc1cc2cc(C(=O)N(Cc3ccncn3)Cc3ccc(C(F)(F)F)cn3)ccc2nc1N] | 2-amino-3-methyl-N-(4-pyrimidinylmethyl)-N-((5-(trifluoromethyl)-2-pyridinyl)methyl)-6-quinolinecarboxamide | 453 |
| 336 | 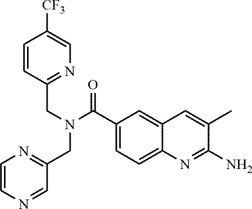 [SMILES: Cc1cc2cc(C(=O)N(Cc3ccc(C(F)(F)F)cn3)Cc3cnccn3)ccc2nc1N] | 2-amino-3-methyl-N-(2-pyrazinylmethyl)-N-((5-(trifluoromethyl)-2-pyridinyl)methyl)-6-quinolinecarboxamide | 453 |
| 337 | 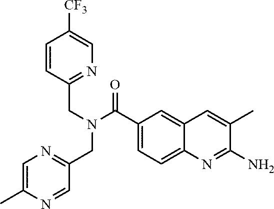 [SMILES: Cc1cnc(CN(Cc2ccc(C(F)(F)F)cn2)C(=O)c2ccc3nc(N)c(C)cc3c2)cn1] | 2-amino-3-methyl-N-((5-methyl-2-pyrazinyl)methyl)-N-((5-(trifluoromethyl)-2-pyridinyl)methyl)-6-quinolinecarboxamide | 467.3 |
| 338 | 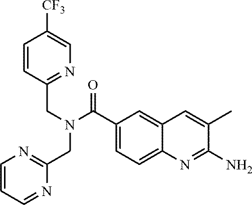 [SMILES: Cc1cc2cc(C(=O)N(Cc3ccc(C(F)(F)F)cn3)Cc3ncccn3)ccc2nc1N] | 2-amino-3-methyl-N-(2-pyrimidinylmethyl)-N-((5-(trifluoromethyl)-2-pyridinyl)methyl)-6-quinolinecarboxamide | 452.8 |
| 339 | 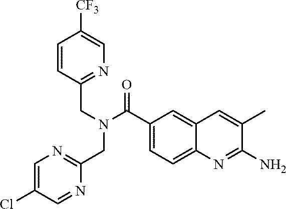 [SMILES: Cc1cc2cc(C(=O)N(Cc3ccc(C(F)(F)F)cn3)Cc3ncc(Cl)cn3)ccc2nc1N] | 2-amino-N-((5-chloro-2-pyrimidinyl)methyl)-3-methyl-N-((5-(trifluoromethyl)-2-pyridinyl)methyl)-6-quinolinecarboxamide | 487.2 |
| 340 | 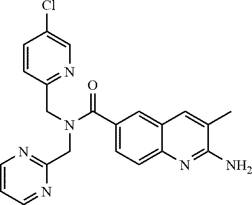 [SMILES: Cc1cc2cc(C(=O)N(Cc3ccc(Cl)cn3)Cc3ncccn3)ccc2nc1N] | 2-amino-N-((5-chloro-2-pyridinyl)methyl)-3-methyl-N-(2-pyrimidinylmethyl)-6-quinolinecarboxamide | 419.2 |
| 341 | 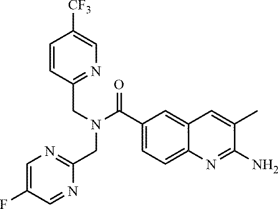 [SMILES: Cc1cc2cc(C(=O)N(Cc3ccc(C(F)(F)F)cn3)Cc3ncc(F)cn3)ccc2nc1N] | 2-amino-N-((5-fluoro-2-pyrimidinyl)methyl)-3-methyl-N-((5-(trifluoromethyl)-2-pyridinyl)methyl)-6-quinolinecarboxamide | 471.1 |
| 342 | 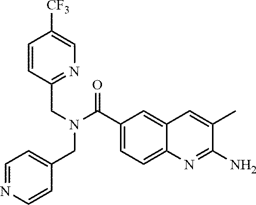 [SMILES: Cc1cc2cc(C(=O)N(Cc3ccncc3)Cc3ccc(C(F)(F)F)cn3)ccc2nc1N] | 2-amino-3-methyl-N-(4-pyridinylmethyl)-N-((5-(trifluoromethyl)-2-pyridinyl)methyl)-6-quinolinecarboxamide | 452 |
| 343 | 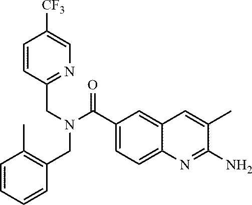 [SMILES: Cc1ccccc1CN(Cc1ccc(C(F)(F)F)cn1)C(=O)c1ccc2nc(N)c(C)cc2c1] | 2-amino-3-methyl-N-((3-methyl-2-pyridinyl)methyl)-N-((5-(trifluoromethyl)-2-pyridinyl)methyl)-6-quinolinecarboxamide | 466.2 |
| 344 | 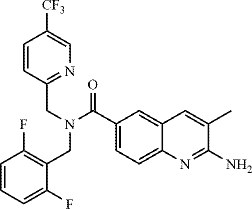 [SMILES: Cc1cc2cc(C(=O)N(Cc3ccc(C(F)(F)F)cn3)Cc3c(F)cccc3F)ccc2nc1N] | 2-amino-N-(2,6-difluorobenzyl)-3-methyl-N-((5-(trifluoromethyl)-2-pyridinyl)methyl)-6-quinolinecarboxamide | 487.2 |

Example 345: (R)-2-amino-3-bromo-N-((5-chloropyridin-2-yl)methyl)-N-(1-(pyrimidin-2-yl)ethyl)quinoline-6-carboxamide. (Method A-PyBroP)

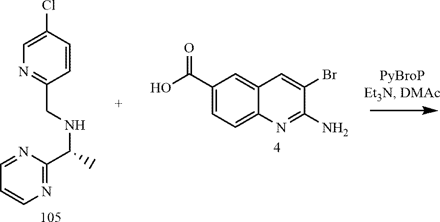

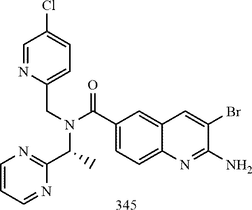

To a mixture of 2-amino-3-bromoquinoline-6-carboxylic acid (4, 64 mg, 0.24 mmol), (R)-N-((5-chloropyridin-2-yl)methyl)-1-(pyrimidin-2-yl)ethan-1-amine (105, 50 mg, 0.20 mmol), and bromotripyrrolidinophosphonium hexafluorophosphate (PyBroP) (112 mg, 0.24 mmol, Sigma-Aldrich Corporation) and N, N-dimethylacetamide (DMAc) (2 mL) was added triethylamine (0.07 mL, 0.50 mmol). The resulting mixture was stirred at RT for 18 h then filtered via a fritted funnel. The solid was rinsed with 1 mL of DMAc. The filtrate was purified by reverse phase prep. HPLC (10-70% water in MeCN with 0.1% TFA) to afford (R)-2-amino-3-bromo-N-((5-chloropyridin-2-yl)methyl)-N-(1-(pyrimidin-2-yl)ethyl)quinoline-6-carboxamide 2,2,2-trifluoroacetate (345, 83 mg, 63% yield) as an off-white solid. m/z (ESI): 497/499 (M+H)⁺. ¹H NMR (400 MHz, METHANOL-d₄) δppm 8.74 (br d, J=3.7 Hz, 2 H), 8.31 - 8.44 (m, 2 H), 7.56 - 7.96 (m, 4 H), 7.27 - 7.45 (m, 2 H), 5.36 - 5.52 (m, 1 H), 4.90 - 5.01 (m, 1 H), 4.55 - 4.68 (m, 1 H), 1.67 (d, J=7.0 Hz, 3 H).

TABLE 9

Examples below were prepared in a manner similar to that described above (Method A-PyBroP) for Example 345

| Ex. # | Chemical Structure | Name | LRMS: (ESI + ve ion) m/z |
|---|---|---|---|
| 346 | 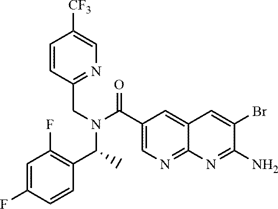 [SMILES: C[C@H](c1ccc(F)cc1F)N(Cc1ccc(C(F)(F)F)cn1)C(=O)c1cnc2nc(N)c(Br)cc2c1] | 7-amino-6-bromo-N-((1R)-1-(2,4-difluorophenyl)ethyl)-N-((5-(trifluoromethyl)-2-pyridinyl)methyl)-1,8-naphthyridine-3-carboxamide | 566/568 |
| 347 | 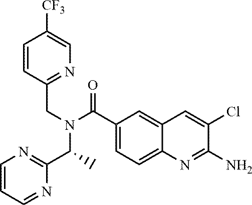 [SMILES: C[C@H](c1ncccn1)N(Cc1ccc(C(F)(F)F)cn1)C(=O)c1ccc2nc(N)c(Cl)cc2c1] | 2-amino-3-chloro-N-((1R)-1-(2-pyrimidinyl)ethyl)-N-((5-(trifluoromethyl)-2-pyridinyl)methyl)-6-quinolinecarboxamide | 487.2 |
| 348 | 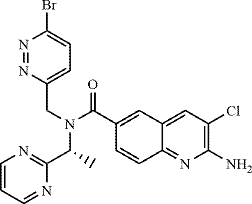 [SMILES: C[C@H](c1ncccn1)N(Cc1ccc(Br)nn1)C(=O)c1ccc2nc(N)c(Cl)cc2c1] | 2-amino-N-((6-bromo-3-pyridazinyl)methyl)-3-chloro-N-((1R)-1-(2-pyrimidinyl)ethyl)-6-quinolinecarboxamide | 498 |
| 349 | 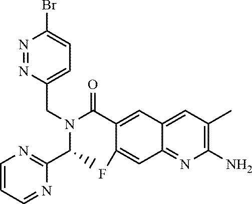 [SMILES: Cc1cc2cc(C(=O)N(Cc3ccc(Br)nn3)[C@H](C)c3ncccn3)c(F)cc2nc1N] | 2-amino-N-((6-bromo-3-pyridazinyl)methyl)-7-fluoro-3-methyl-N-((1R)-1-(2-pyrimidinyl)ethyl)-6-quinolinecarboxamide | 496.2 |
| 350 | 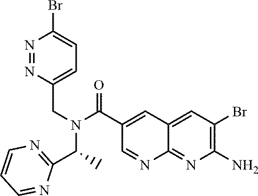 [SMILES: C[C@H](c1ncccn1)N(Cc1ccc(Br)nn1)C(=O)c1cnc2nc(N)c(Br)cc2c1] | 7-amino-6-bromo-N-((6-bromo-3-pyridazinyl)methyl)-N-((1R)-1-(2-pyrimidinyl)ethyl)-1,8-naphthyridine-3-carboxamide | 545.8 |
| 351 | 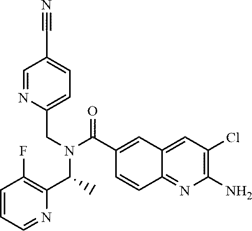 [SMILES: C[C@H](c1ncccc1F)N(Cc1ccc(C#N)cn1)C(=O)c1ccc2nc(N)c(Cl)cc2c1] | 2-amino-3-chloro-N-((5-cyano-2-pyridinyl)methyl)-N-((1R)-1-(3-fluoro-2-pyridinyl)ethyl)-6-quinolinecarboxamide | 461.2 |
| 352 | 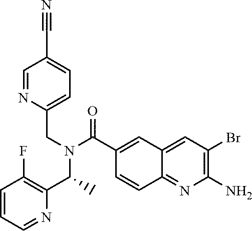 [SMILES: C[C@H](c1ncccc1F)N(Cc1ccc(C#N)cn1)C(=O)c1ccc2nc(N)c(Br)cc2c1] | 2-amino-3-bromo-N-((5-cyano-2-pyridinyl)methyl)-N-((1R)-1-(3-fluoro-2-pyridinyl)ethyl)-6-quinolinecarboxamide | 505/507 |
| 353 | 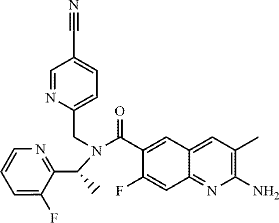 [SMILES: Cc1cc2cc(C(=O)N(Cc3ccc(C#N)cn3)[C@H](C)c3ncccc3F)c(F)cc2nc1N] | 2-amino-N-((5-cyano-2-pyridinyl)methyl)-7-fluoro-N-((1R)-1-(3-fluoro-2-pyridinyl)ethyl)-3-methyl-6-quinolinecarboxamide | 459.2 |
| 354 | 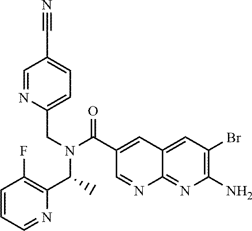 [SMILES: C[C@H](c1ncccc1F)N(Cc1ccc(C#N)cn1)C(=O)c1cnc2nc(N)c(Br)cc2c1] | 7-amino-6-bromo-N-((5-cyano-2-pyridinyl)methyl)-N-((1R)-1-(3-fluoro-2-pyridinyl)ethyl)-1,8-naphthyridine-3-carboxamide | 506/508 |
| 355 | 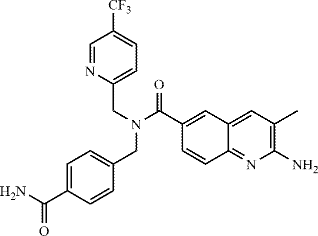 [SMILES: Cc1cc2cc(C(=O)N(Cc3ccc(C(N)=O)cc3)Cc3ccc(C(F)(F)F)cn3)ccc2nc1N] | 2-amino-N-(4-carbamoylbenzyl)-3-methyl-N-((5-(trifluoromethyl)-2-pyridinyl)methyl)-6-quinolinecarboxamide | 494 |
| 356 | 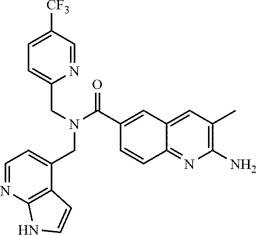 [SMILES: Cc1cc2cc(C(=O)N(Cc3ccc(C(F)(F)F)cn3)Cc3ccnc4[nH]ccc34)ccc2nc1N] | 2-amino-3-methyl-N-(1H-pyrrolo[2,3-b]pyridin-4-ylmethyl)-N-((5-(trifluoromethyl)-2-pyridinyl)methyl)-6-quinolinecarboxamide | 491.1 |
| 357 | 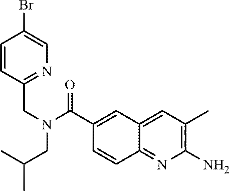 [SMILES: Cc1cc2cc(C(=O)N(Cc3ccc(Br)cn3)CC(C)C)ccc2nc1N] | 2-amino-N-((5-bromo-2-pyridinyl)methyl)-3-methyl-N-(2-methylpropyl)-6-quinolinecarboxamide | 427/429 |
| 358 | 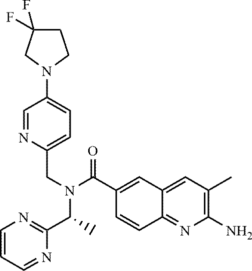 [SMILES: Cc1cc2cc(C(=O)N(Cc3ccc(N4CCC(F)(F)C4)cn3)[C@H](C)c3ncccn3)ccc2nc1N] | 2-amino-N-((5-(3,3-difluoro-1-pyrrolidinyl)-2-pyridinyl)methyl)-3-methyl-N-((1R)-1-(2-pyrimidinyl)ethyl)-6-quinolinecarboxamide | 504.2 |
| 359 | 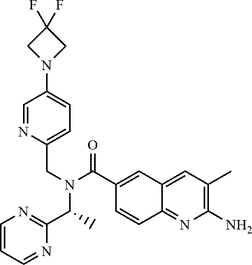 [SMILES: Cc1cc2cc(C(=O)N(Cc3ccc(N4CC(F)(F)C4)cn3)[C@H](C)c3ncccn3)ccc2nc1N] | 2-amino-N-((5-(3,3-difluoro-1-azetidinyl)-2-pyridinyl)methyl)-3-methyl-N-((1R)-1-(2-pyrimidinyl)ethyl)-6-quinolinecarboxamide | 490.2 |
| 360 | 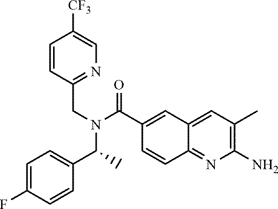 [SMILES: Cc1cc2cc(C(=O)N(Cc3ccc(C(F)(F)F)cn3)[C@H](C)c3ccc(F)cc3)ccc2nc1N] | 2-amino-N-((1R)-1-(4-fluorophenyl)ethyl)-3-methyl-N-((5-(trifluoromethyl)-2-pyridinyl)methyl)-6-quinolinecarboxamide | 483.2 |
| 361 | 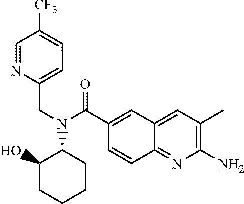 [SMILES: Cc1cc2cc(C(=O)N(Cc3ccc(C(F)(F)F)cn3)[C@@H]3CCCC[C@H]3O)ccc2nc1N] | 2-amino-N-((1R,2R)-2-hydroxycyclohexyl)-3-methyl-N-((5-(trifluoromethyl)-2-pyridinyl)methyl)-6-quinolinecarboxamide | 459.2 |
| 362 | 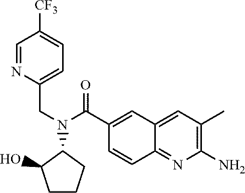 [SMILES: Cc1cc2cc(C(=O)N(Cc3ccc(C(F)(F)F)cn3)[C@@H]3CCC[C@H]3O)ccc2nc1N] | 2-amino-N-((1R,2R)-2-hydroxycyclopentyl)-3-methyl-N-((5-(trifluoromethyl)-2-pyridinyl)methyl)-6-quinolinecarboxamide | 445.5 |
| 363 | 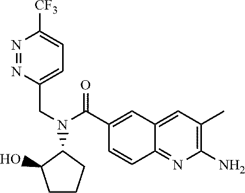 [SMILES: Cc1cc2cc(C(=O)N(Cc3ccc(C(F)(F)F)nn3)[C@@H]3CCC[C@H]3O)ccc2nc1N] | 2-amino-N-((1R,2R)-2-hydroxycyclopentyl)-3-methyl-N-((6-(trifluoromethyl)pyridazin-3-yl)methyl)quinoline-6-carboxamide | 446.2 |
| 364 | 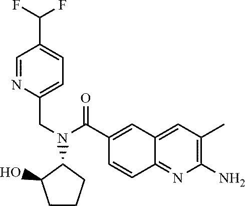 [SMILES: Cc1cc2cc(C(=O)N(Cc3ccc(C(F)F)cn3)[C@@H]3CCC[C@H]3O)ccc2nc1N] | 2-amino-N-((5-(difluoromethyl)pyridin-2-yl)methyl)-N-((1R,2R)-2-hydroxycyclopentyl)-3-methylquinoline-6-carboxamide | 427.2 |
| 365 | 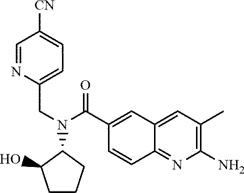 [SMILES: Cc1cc2cc(C(=O)N(Cc3ccc(C#N)cn3)[C@@H]3CCC[C@H]3O)ccc2nc1N] | 2-amino-N-((5-cyanopyridin-2-yl)methyl)-N-((1R,2R)-2-hydroxycyclopentyl)-3-methylquinoline-6-carboxamide | 402.2 |
| 366 | 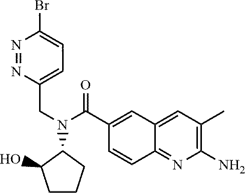 [SMILES: Cc1cc2cc(C(=O)N(Cc3ccc(Br)nn3)[C@@H]3CCC[C@H]3O)ccc2nc1N] | 2-amino-N-((6-bromo-3-pyridazinyl)methyl)-N-((1R,2R)-2-hydroxycyclopentyl)-3-methyl-6-quinolinecarboxamide | 456/458 |
| 367 | 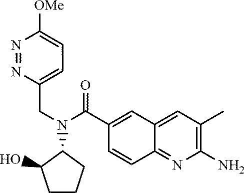 [SMILES: COc1ccc(CN(C(=O)c2ccc3nc(N)c(C)cc3c2)[C@@H]2CCC[C@H]2O)nn1] | 2-amino-N-((1R,2R)-2-hydroxycyclopentyl)-N-((6-methoxypyridazin-3-yl)methyl)-3-methylquinoline-6-carboxamide | 408.2 |
| 368 | 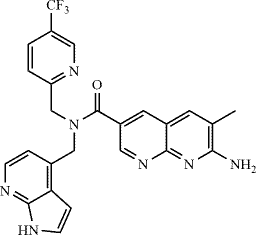 [SMILES: Cc1cc2cc(C(=O)N(Cc3ccc(C(F)(F)F)cn3)Cc3ccnc4[nH]ccc34)cnc2nc1N] | N-((1H-pyrrolo[2,3-b]pyridin-4-yl)methyl)-7-amino-6-methyl-N-((5-(trifluoromethyl)pyridin-2-yl)methyl)-1,8-naphthyridine-3 -carboxamide | 492.2 |
| 369 | 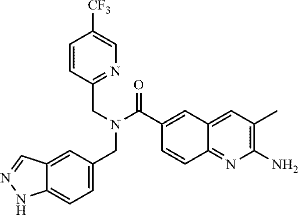 [SMILES: Cc1cc2cc(C(=O)N(Cc3ccc4[nH]ncc4c3)Cc3ccc(C(F)(F)F)cn3)ccc2nc1N] | 2-amino-N-(1H-indazol-5-ylmethyl)-3-methyl-N-((5-(trifluoromethyl)-2-pyridinyl)methyl)-6-quinolinecarboxamide | 491 |
| 370 | 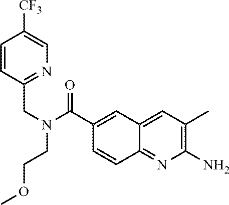 [SMILES: COCCN(Cc1ccc(C(F)(F)F)cn1)C(=O)c1ccc2nc(N)c(C)cc2c1] | 2-amino-N-(2-methoxyethyl)-3-methyl-N-((5-(trifluoromethyl)-2-pyridinyl)methyl)-6-quinolinecarboxamide | 419.2 |
| 371 | 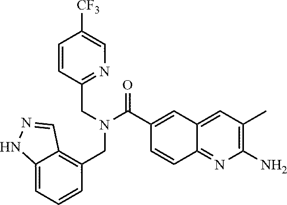 [SMILES: Cc1cc2cc(C(=O)N(Cc3ccc(C(F)(F)F)cn3)Cc3cccc4[nH]ncc34)ccc2nc1N] | 2-amino-N-(1H-indazol-4-ylmethyl)-3-methyl-N-((5-(trifluoromethyl)-2-pyridinyl)methyl)-6-quinolinecarboxamide | 491 |
| 372 | 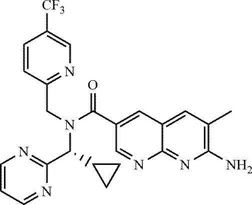 [SMILES: Cc1cc2cc(C(=O)N(Cc3ccc(C(F)(F)F)cn3)[C@@H](c3ncccn3)C3CC3)cnc2nc1N] | 7-amino-N-((R)-cyclopropyl(2-pyrimidinyl)methyl)-6-methyl-N-((5-(trifluoromethyl)-2-pyridinyl)methyl)-1,8-naphthyridine-3-carboxamide | 494.1 |
| 373 | 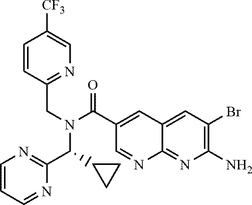 [SMILES: Nc1nc2ncc(C(=O)N(Cc3ccc(C(F)(F)F)cn3)[C@@H](c3ncccn3)C3CC3)cc2cc1Br] | 7-amino-6-bromo-N-((R)-cyclopropyl(2-pyrimidinyl)methyl)-N-((5-(trifluoromethyl)-2-pyridinyl)methyl)-1,8-naphthyridine-3-carboxamide | 558/560 |
| 374 | 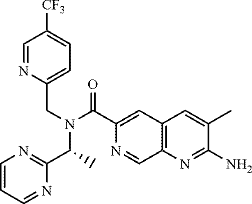 [SMILES: Cc1cc2cc(C(=O)N(Cc3ccc(C(F)(F)F)cn3)[C@H](C)c3ncccn3)ncc2nc1N] | 2-amino-3-methyl-N-((1R)-1-(2-pyrimidinyl)ethyl)-N-((5-(trifluoromethyl)-2-pyridinyl)methyl)-1,7-naphthyridine-6-carboxamide | 468 |
| 375 | 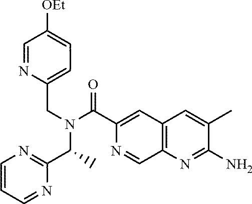 [SMILES: CCOc1ccc(CN(C(=O)c2cc3cc(C)c(N)nc3cn2)[C@H](C)c2ncccn2)nc1] | 2-amino-N-((5-ethoxy-2-pyridinyl)methyl)-3-methyl-N-((1R)-1-(2-pyrimidinyl)ethyl)-6-quinolinecarboxamide | 443.3 |
| 376 | 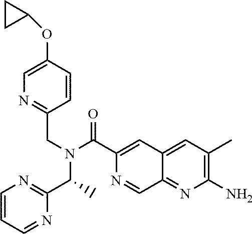 [SMILES: Cc1cc2cc(C(=O)N(Cc3ccc(OC4CC4)cn3)[C@H](C)c3ncccn3)ncc2nc1N] | 2-amino-N-((5-(cyclopropyloxy)-2-pyridinyl)methyl)-3-methyl-N-((1R)-1-(2-pyrimidinyl)ethyl)-6-quinolinecarboxamide | 455 |
| 377 | 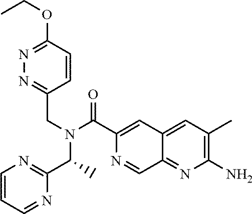 [SMILES: CCOc1ccc(CN(C(=O)c2cc3cc(C)c(N)nc3cn2)[C@H](C)c2ncccn2)nn1] | 2-amino-N-((6-ethoxy-3-pyridazinyl)methyl)-3-methyl-N-((1R)-1-(2-pyrimidinyl)ethyl)-6-quinolinecarboxamide | 444.2 |
| 378 | 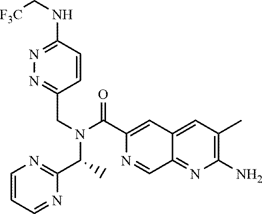 [SMILES: Cc1cc2cc(C(=O)N(Cc3ccc(NCC(F)(F)F)nn3)[C@H](C)c3ncccn3)ncc2nc1N] | 2-amino-3-methyl-N-((1R)-1-(2-pyrimidinyl)ethyl)-N-((6-((2,2,2-trifluoroethyl)amino)-3-pyridazinyl)methyl)-6-quinolinecarboxamide | 497.1 |
| 379 | 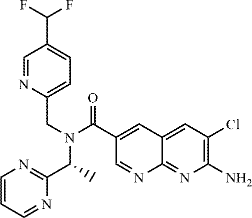 [SMILES: C[C@H](c1ncccn1)N(Cc1ccc(C(F)F)cn1)C(=O)c1cnc2nc(N)c(Cl)cc2c1] | 7-amino-6-chloro-N-((5-(difluoromethyl)-2-pyridinyl)methyl)-N-((1R)-1-(2-pyrimidinyl)ethyl)-1),8-naphthyridine-3-carboxamide | 470 |
| 380 | 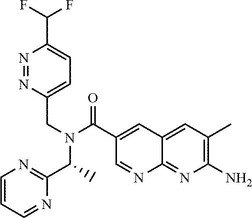 [SMILES: Cc1cc2cc(C(=O)N(Cc3ccc(C(F)F)nn3)[C@H](C)c3ncccn3)cnc2nc1N] | 2-amino-N-((6-(difluoromethyl)-3-pyridazinyl)methyl)-3-methyl-N-((1R)-1-(2-pyrimidinyl)ethyl)-6-quinolinecarboxamide | 450 |
| 381 | 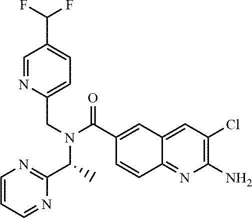 [SMILES: C[C@H](c1ncccn1)N(Cc1ccc(C(F)F)cn1)C(=O)c1ccc2nc(N)c(Cl)cc2c1] | 2-amino-3-chloro-N-((5-(difluoromethyl)-2-pyridinyl)methyl)-N-((1R)-1-(2-pyrimidinyl)ethyl)-6-quinolinecarboxamide | 469.1 |
| 382 | 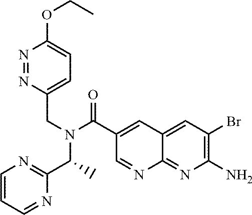 [SMILES: CCOc1ccc(CN(C(=O)c2cnc3nc(N)c(Br)cc3c2)[C@H](C)c2ncccn2)nn1] | 7-amino-6-bromo-N-((6-ethoxy-3-pyridazinyl)methyl)-N-((1R)-1-(2-pyrimidinyl)ethyl)-1,8-naphthyridine-3-carboxamide | 509.0/511.1 |
| 383 | 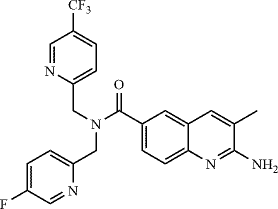 [SMILES: Cc1cc2cc(C(=O)N(Cc3ccc(F)cn3)Cc3ccc(C(F)(F)F)cn3)ccc2nc1N] | 2-amino-N-((5-fluoro-2-pyridinyl)methyl)-3-methyl-N-((5-(trifluoromethyl)-2-pyridinyl)methyl)-6-quinolinecarboxamide | 470.1 |
| 384 | 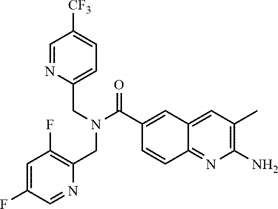 [SMILES: Cc1cc2cc(C(=O)N(Cc3ccc(C(F)(F)F)cn3)Cc3ncc(F)cc3F)ccc2nc1N] | 2-amino-N-((3,5-difluoro-2-pyridinyl)methyl)-3-methyl-N-((5-(trifluoromethyl)-2-pyridinyl)methyl)-6-quinolinecarboxamide | 488.2 |
| 385 | 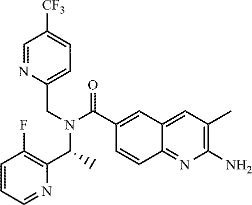 [SMILES: Cc1cc2cc(C(=O)N(Cc3ccc(C(F)(F)F)cn3)[C@H](C)c3ncccc3F)ccc2nc1N] | 2-amino-N-((1R)-1-(3-fluoro-2-pyridinyl)ethyl)-3-methyl-N-((5-(trifluoromethyl)-2-pyridinyl)methyl)-6-quinolinecarboxamide | 484.1 |
| 386 | 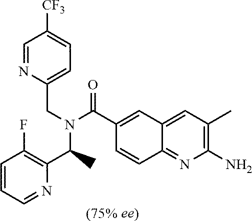 | 2-amino-N-((1S)-1-(3-fluoro-2-pyridinyl)ethyl)-3-methyl-N-((5-(trifluoromethyl)-2-pyridinyl)methyl)-6-quinolinecarboxamide | 484.1 |
| 387 | 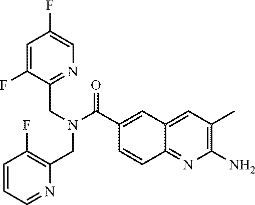 [SMILES: Cc1cc2cc(C(=O)N(Cc3ncccc3F)Cc3ncc(F)cc3F)ccc2nc1N] | 2-amino-N-((3,5-difluoro-2-pyridinyl)methyl)-N-((3-fluoro-2-pyridinyl)methyl)-3-methyl-6-quinolinecarboxamide | 438.2 |
| 388 | 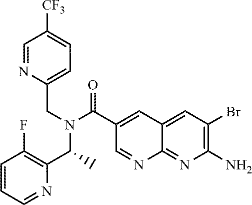 [SMILES: C[C@H](c1ncccc1F)N(Cc1ccc(C(F)(F)F)cn1)C(=O)c1cnc2nc(N)c(Br)cc2c1] | 7-amino-6-bromo-N-((1R)-1-(3-fluoro-2-pyridinyl)ethyl)-N-((5-(trifluoromethyl)-2-pyridinyl)methyl)-1,8-naphthyridine-3-carboxamide | 549.0/551.0 |
| 389 | 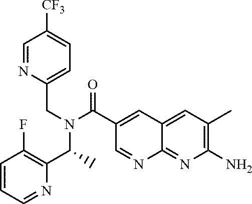 [SMILES: Cc1cc2cc(C(=O)N(Cc3ccc(C(F)(F)F)cn3)[C@H](C)c3ncccc3F)cnc2nc1N] | 7-amino-N-((1R)-1-(3-fluoro-2-pyridinyl)ethyl)-6-methyl-N-((5-(trifluoromethyl)-2-pyridinyl)methyl)-1,8-naphthyridine-3-carboxamide | 485.1 |
| 390 | 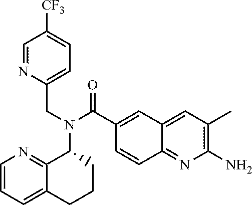 [SMILES: Cc1cc2cc(C(=O)N(Cc3ccc(C(F)(F)F)cn3)[C@@H]3CCCc4cccnc43)ccc2nc1N] | 2-amino-3-methyl-N-((8R)-5,6,7,8-tetrahydro-8-quinolinyl)-N-((5-(trifluoromethyl)-2-pyridinyl)methyl)-6-quinolinecarboxamide | 492.2 |
| 391 | 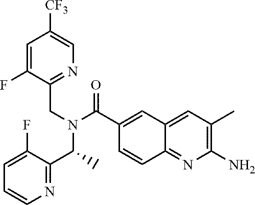 [SMILES: Cc1cc2cc(C(=O)N(Cc3ncc(C(F)(F)F)cc3F)[C@H](C)c3ncccc3F)ccc2nc1N] | 2-amino-N-((1R)-1-(3-fluoro-2-pyridinyl)ethyl)-N-((3-fluoro-5-(trifluoromethyl)-2-pyridinyl)methyl)-3-methyl-6-quinolinecarboxamide | 502.1 |
| 392 | 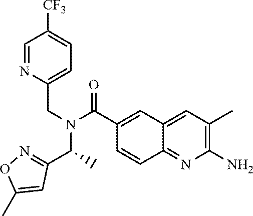 [SMILES: Cc1cc([C@@H](C)N(Cc2ccc(C(F)(F)F)cn2)C(=O)c2ccc3nc(N)c(C)cc3c2)no1] | 2-amino-3-methyl-N-((1R)-1-(5-methyl-1,2-oxazol-3-yl)ethyl)-N-((5-(trifluoromethyl)-2-pyridinyl)methyl)-6-quinolinecarboxamide | 470.1 |
| 393 | 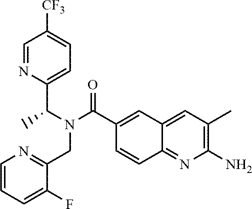 [SMILES: Cc1cc2cc(C(=O)N(Cc3ncccc3F)[C@H](C)c3ccc(C(F)(F)F)cn3)ccc2nc1N] | 2-amino-N-((3-fluoro-2-pyridinyl)methyl)-3-methyl-N-((1R)-1-(5-(trifluoromethyl)-2-pyridinyl)ethyl)-6-quinolinecarboxamide | 484.1 |
| 394 | 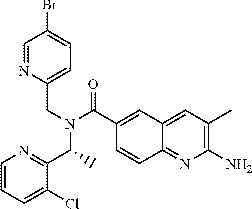 [SMILES: Cc1cc2cc(C(=O)N(Cc3ccc(Br)cn3)[C@H](C)c3ncccc3Cl)ccc2nc1N] | 2-amino-N-((5-bromo-2-pyridinyl)methyl)-N-((1R)-1-(3-chloro-2-pyridinyl)ethyl)-3-methyl-6-quinolinecarboxamide | 510/512 |
| 395 | 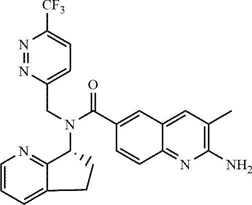 [SMILES: Cc1cc2cc(C(=O)N(Cc3ccc(C(F)(F)F)nn3)[C@@H]3CCc4cccnc43)ccc2nc1N] | 2-amino-N-((7R)-6,7-dihydro-5H-cyclopenta[b]pyridin-7-yl)-3-methyl-N-((6-(trifluoromethyl)-3-pyridazinyl)methyl)-6-quinolinecarboxamide | 479 |
| 396 | 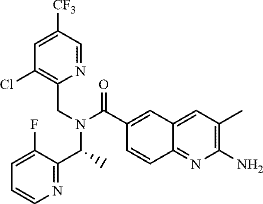 [SMILES: Cc1cc2cc(C(=O)N(Cc3ncc(C(F)(F)F)cc3Cl)[C@H](C)c3ncccc3F)ccc2nc1N] | 2-amino-N-((3-chloro-5-(trifluoromethyl)-2-pyridinyl)methyl)-N-((1R)-1-(3-fluoro-2-pyridinyl)ethyl)-3-methyl-6-quinolinecarboxamide | 518 |
| 397 | 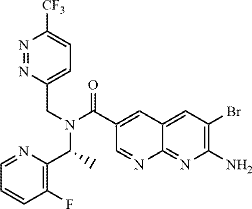 [SMILES: C[C@H](c1ncccc1F)N(Cc1ccc(C(F)(F)F)nn1)C(=O)c1cnc2nc(N)c(Br)cc2c1] | 7-amino-6-bromo-N-((1R)-1-(3-fluoro-2-pyridinyl)ethyl)-N-((6-(trifluoromethyl)-3-pyridazinyl)methyl)-1,8-naphthyridine-3-carboxamide | 550.0/552.0 |
| 398 | 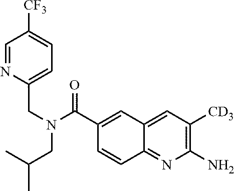 [SMILES: [2H]C([2H])([2H])c1cc2cc(C(=O)N(Cc3ccc(C(F)(F)F)cn3)CC(C)C)ccc2nc1N] | 2-amino-N-isobutyl-3-(methyl-d₃)-N-((5- (trifluoromethyl)pyridin-2-yl)methyl)quinoline-6-carboxamide | 420.2 |
| 399 | 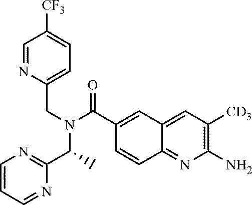 [SMILES: [2H]C([2H])([2H])c1cc2cc(C(=O)N(Cc3ccc(C(F)(F)F)cn3)[C@H](C)c3ncccn3)ccc2nc1N] | (R)-2-amino-3-(methyl-d₃)-N-(1-(pyrimidin-2-yl)ethyl)-N-((5-(trifluoromethyl)pyridin-2-yl)methyl)quinoline-6-carboxamide | 470.1 |
| 400 | 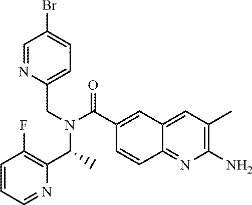 [SMILES: Cc1cc2cc(C(=O)N(Cc3ccc(Br)cn3)[C@H](C)c3ncccc3F)ccc2nc1N] | 2-amino-N-((5-bromo-2-pyridinyl)methyl)-N-((1R)-1-(3 -fluoro-2-pyridinyl)ethyl)-3 -methyl-6-quinolinecarboxamide | 494.0/496.1 |
| 401 | 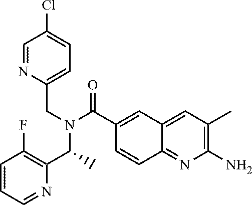 [SMILES: Cc1cc2cc(C(=O)N(Cc3ccc(Cl)cn3)[C@H](C)c3ncccc3F)ccc2nc1N] | 2-amino-N-((5-chloro-2-pyridinyl)methyl)-N-((1R)-1-(3 -fluoro-2-pyridinyl)ethyl)-3 -methyl-6-quinolinecarboxamide | 450 |
| 402 | 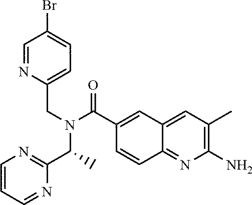 [SMILES: Cc1cc2cc(C(=O)N(Cc3ccc(Br)cn3)[C@H](C)c3ncccn3)ccc2nc1N] | 2-amino-N-((5-bromo-2-pyridinyl)methyl)-3-methyl-N-((1R)-1-(2-pyrimidinyl)ethyl-6-quinolinecarboxamide | 477/479 |
| 403 | 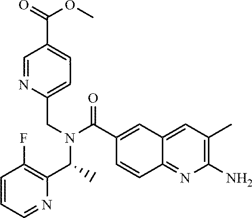 [SMILES: COC(=O)c1ccc(CN(C(=O)c2ccc3nc(N)c(C)cc3c2)[C@H](C)c2ncccc2F)nc1] | methyl 6-((((2-amino-3-methyl-6-quinolinyl)carbonyl)((1R)-1-(3-fluoro-2-pyridinyl)ethyl)amino)methyl)-3-pyridinecarboxylate | 474 |
| 404 | 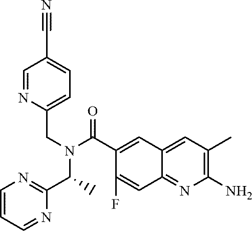 [SMILES: Cc1cc2cc(C(=O)N(Cc3ccc(C#N)cn3)[C@H](C)c3ncccn3)c(F)cc2nc1N] | 2-amino-N-((5-cyano-2-pyridinyl)methyl)-7-fluoro-3-methyl-N-((1R)-1-(2-pyrimidinyl)ethyl)-6-quinolinecarboxamide | 442 |
| 405 | 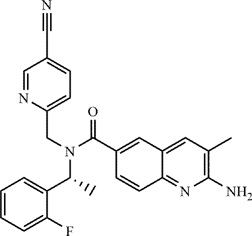 [SMILES: Cc1cc2cc(C(=O)N(Cc3ccc(C#N)cn3)[C@H](C)c3ccccc3F)ccc2nc1N] | 2-amino-N-((5-cyano-2-pyridinyl)methyl)-N-((1R)-1-(2-fluorophenyl)ethyl)-3-methyl-6-quinolinecarboxamide | 440 |
| 406 | 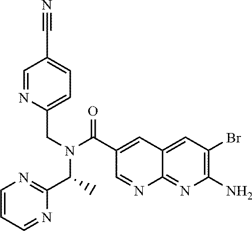 [SMILES: C[C@H](c1ncccn1)N(Cc1ccc(C#N)cn1)C(=O)c1cnc2nc(N)c(Br)cc2c1] | 7-amino-6-bromo-N-((5-cyano-2-pyridinyl)methyl)-N-((1R)-1-(2-pyrimidinyl)ethyl)-1,8-naphthyridine-3-carboxamide | 489/91 |
| 407 | 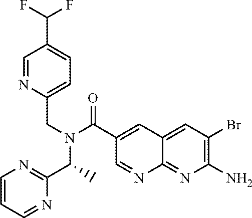 [SMILES: C[C@H](c1ncccn1)N(Cc1ccc(C(F)F)cn1)C(=O)c1cnc2nc(N)c(Br)cc2c1] | 7-amino-6-bromo-N-((5-(difluoromethyl)-2-pyridinyl)methyl)-N-((1R)-1-(2-pyrimidinyl)ethyl)-1,8-naphthyridine-3-carboxamide | 513 515 |
| 408 | 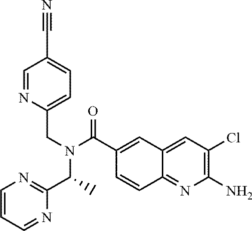 [SMILES: C[C@H](c1ncccn1)N(Cc1ccc(C#N)cn1)C(=O)c1ccc2nc(N)c(Cl)cc2c1] | 2-amino-3-chloro-N-((5-cyano-2-pyridinyl)methyl)-N-((1R)-1-(2-pyrimidinyl)ethyl)-6-quinolinecarboxamide | 444 |
| 409 | 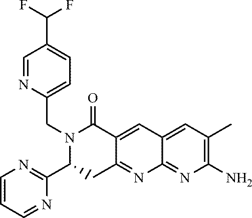 [SMILES: Cc1cc2cc3c(nc2nc1N)C[C@H](c1ncccn1)N(Cc1ccc(C(F)F)cn1)C3=O] | 7-amino-N-((5-(difluoromethyl)-2-pyridinyl)methyl)-6-methyl-N-((1R)-1-(2-pyrimidinyl)ethyl)-1,8-naphthyridine-3-carboxamide | 450 |
| 410 | 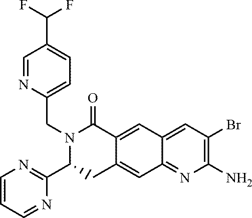 [SMILES: Nc1nc2cc3c(cc2cc1Br)C(=O)N(Cc1ccc(C(F)F)cn1)[C@@H](c1ncccn1)C3] | 2-amino-3-bromo-N-((5-(difluoromethyl)-2-pyridinyl)methyl)-N-((1R)-1-(2-pyrimidinyl)ethyl)-6-quinolinecarboxamide | 513/515 |
| 411 | 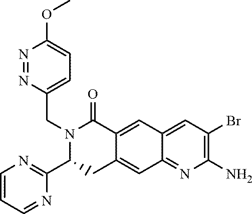 [SMILES: COc1ccc(CN2C(=O)c3cc4cc(Br)c(N)nc4cc3C[C@@H]2c2ncccn2)nn1] | 2-amino-3-bromo-N-((6-methoxy-3-pyridazinyl)methyl)-N-((1R)-1-(2-pyrimidinyl)ethyl)-6-quinolinecarboxamide | 494/496 |
| 412 | 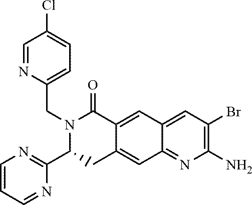 [SMILES: Nc1nc2cc3c(cc2cc1Br)C(=O)N(Cc1ccc(Cl)cn1)[C@@H](c1ncccn1)C3] | 2-amino-3-bromo-N-((5-chloro-2-pyridinyl)methyl)-N-((1R)-1-(2-pyrimidinyl)ethyl)-6-quinolinecarboxamide | 497/499 |
| 413 | 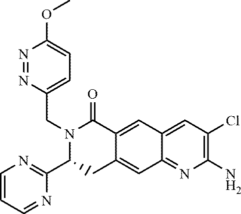 [SMILES: COc1ccc(CN2C(=O)c3cc4cc(Cl)c(N)nc4cc3C[C@@H]2c2ncccn2)nn1] | 2-amino-3-chloro-N-((6-methoxy-3-pyridazinyl)methyl)-N-((1R)-1-(2-pyrimidinyl)ethyl)-6-quinolinecarboxamide | 450 |
| 414 | 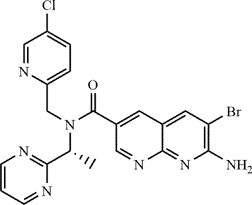 [SMILES: C[C@H](c1ncccn1)N(Cc1ccc(Cl)cn1)C(=O)c1cnc2nc(N)c(Br)cc2c1] | 7-amino-6-bromo-N-((5-chloro-2-pyridinyl)methyl)-N-((1R)-1-(2-pyrimidinyl)ethyl)-1,8-naphthyridine-3-carboxamide | 498/500 |
| 415 | 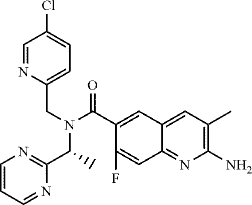 [SMILES: Cc1cc2cc(C(=O)N(Cc3ccc(Cl)cn3)[C@H](C)c3ncccn3)c(F)cc2nc1N] | 2-amino-N-((5-chloro-2-pyridinyl)methyl)-7-fluoro-3-methyl-N-((1R)-1-(2-pyrimidinyl)ethyl)-6-quinolinecarboxamide | 451 |
| 416 | 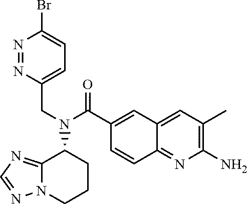 [SMILES: Cc1cc2cc(C(=O)N(Cc3ccc(Br)nn3)[C@@H]3CCCn4ncnc43)ccc2nc1N] | 2-amino-N-((6-bromo-3-pyridazinyl)methyl)-3-methyl-N-((8R)-5,6,7,8-tetrahydro[1,2,4]triazolo[1,5-a]pyridin-8-yl)-6-quinolinecarboxamide | 493/495 |
| 417 | 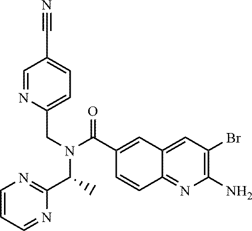 [SMILES: C[C@H](c1ncccn1)N(Cc1ccc(C#N)cn1)C(=O)c1ccc2nc(N)c(Br)cc2c1] | 2-amino-3-bromo-N-((5-cyano-2-pyridinyl)methyl)-N-((1R)-1-(2-pyrimidinyl)ethyl)-6-quinolinecarboxamide | 488/490 |
| 418 | 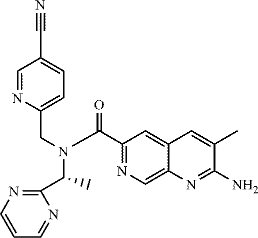 [SMILES: Cc1cc2cc(C(=O)N(Cc3ccc(C#N)cn3)[C@H](C)c3ncccn3)ncc2nc1N] | 2-amino-N-((5-cyano-2-pyridinyl)methyl)-3-methyl-N-((1R)-1-(2-pyrimidinyl)ethyl)-1,7-naphthyridine-6-carboxamide | 425.1 |
| 419 | 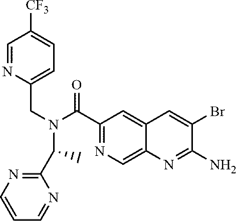 [SMILES: C[C@H](c1ncccn1)N(Cc1ccc(C(F)(F)F)cn1)C(=O)c1cc2cc(Br)c(N)nc2cn1] | 2-amino-3-bromo-N-((1R)-1-(2-pyrimidinyl)ethyl)-N-((5-(trifluoromethyl)-2-pyridinyl)methyl)-1,7-naphthyridine-6-carboxamide | 532.0/534.0 |
| 420 | 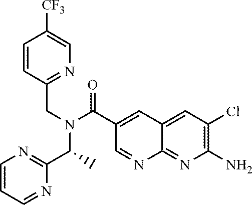 [SMILES: C[C@H](c1ncccn1)N(Cc1ccc(C(F)(F)F)cn1)C(=O)c1cnc2nc(N)c(Cl)cc2c1] | 7-amino-6-chloro-N-((1R)-1-(2-pyrimidinyl)ethyl)-N-((5-(trifluoromethyl)-2-pyridiny1)methyl)-1,8-naphthyridine-3-carboxamide | 490 |
| 421 | 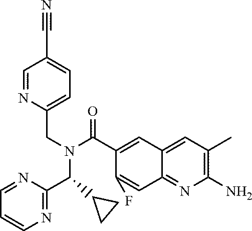 [SMILES: Cc1cc2cc(C(=O)N(Cc3ccc(C#N)cn3)[C@@H](c3ncccn3)C3CC3)c(F)cc2nc1N] | 2-amino-N-((5-cyano-2-pyridinyl)methyl)-N-((R)-cyclopropyl(2-pyrimidinyl)methyl)-7-fluoro-3-methyl-6-quinolinecarboxamide | 468 |
| 422 | 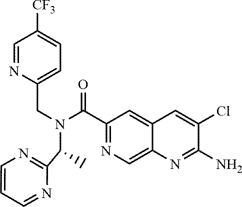 [SMILES: C[C@H](c1ncccn1)N(Cc1ccc(C(F)(F)F)cn1)C(=O)c1cc2cc(Cl)c(N)nc2cn1] | 2-amino-3-chloro-N-((1R)-1-(2-pyrimidinyl)ethyl)-N-((5-(trifluoromethyl)-2-pyridinyl)methyl)-1,7-naphthyridine-6-carboxamide | 488.0 |
| 423 | 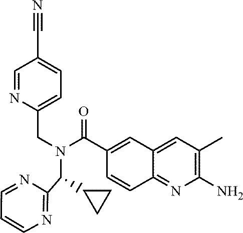 [SMILES: Cc1cc2cc(C(=O)N(Cc3ccc(C#N)cn3)[C@@H](c3ncccn3)C3CC3)ccc2nc1N] | 2-amino-N-((5-cyano-2-pyridinyl)methyl)-N-((R)-cyclopropyl(2-pyrimidinyl)methyl)-3-methyl-6-quinolinecarboxamide | 450 |
| 424 | 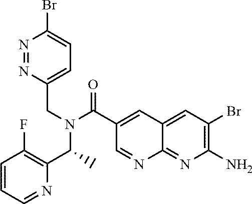 [SMILES: C[C@H](c1ncccc1F)N(Cc1ccc(Br)nn1)C(=O)c1cnc2nc(N)c(Br)cc2c1] | 7-amino-6-bromo-N-((6-bromo-3-pyridazinyl)methyl)-N-((1R)-1-(3-fluoro-2-pyridinyl)ethyl)-1,8-naphthyridine-3-carboxamide | 561.8 |
| 425 | 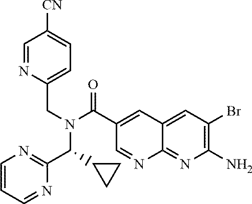 [SMILES: N#Cc1ccc(CN(C(=O)c2cnc3nc(N)c(Br)cc3c2)[C@@H](c2ncccn2)C2CC2)nc1] | (R)-7-amino-6-bromo-N-((5-cyanopyridin-2-yl)methyl)-N-(cyclopropyl(pyrimidin-2-yl)methyl)-1,8-naphthyridine-3-carboxamide | 514.8/516.8 |
| 426 | 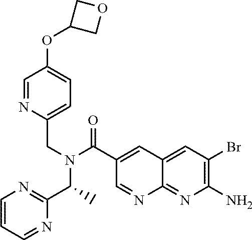 [SMILES: C[C@H](c1ncccn1)N(Cc1ccc(OC2COC2)cn1)C(=O)c1cnc2nc(N)c(Br)cc2c1] | 7-amino-6-bromo-N-((5-(3-oxetanyloxy)-2-pyridinyl)methyl)-N-((1R)-1-(2-pyrimidinyl)ethyl)-1,8-naphthyridine-3-carboxamide | 536/538 |
| 427 | 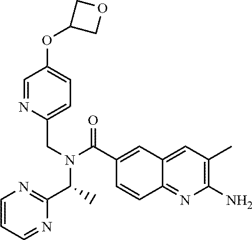 [SMILES: Cc1cc2cc(C(=O)N(Cc3ccc(OC4COC4)cn3)[C@H](C)c3ncccn3)ccc2nc1N] | 2-amino-3-methyl-N-((5-(3-oxetanyloxy)-2-pyridinyl)methyl)-N-((1R)-1-(2-pyrimidinyl)ethyl)-6-quinolinecarboxamide | 471 |
| 428 | 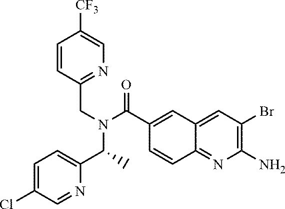 [SMILES: C[C@H](c1ccc(Cl)cn1)N(Cc1ccc(C(F)(F)F)cn1)C(=O)c1ccc2nc(N)c(Br)cc2c1] | 2-amino-N-((1R)-1-(5-chloro-2-pyridinyl)ethyl)-3-methyl-N-((5-(trifluoromethyl)-2-pyridinyl)methyl)-6-quinolinecarboxamide | 500.3 |
| 429 | 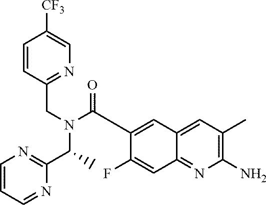 [SMILES: Cc1cc2cc(C(=O)N(Cc3ccc(C(F)(F)F)cn3)[C@H](C)c3ncccn3)c(F)cc2nc1N] | 2-amino-7-fluoro-3-methyl-N-((1R)-1-(2-pyrimidinyl)ethyl)-N-((5-(trifluoromethyl)-2-pyridinyl)methyl)-6-quinolinecarboxamide | 485.1 |
| 430 | 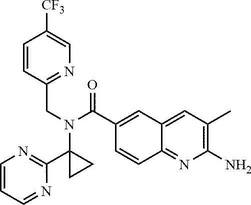 [SMILES: Cc1cc2cc(C(=O)N(Cc3ccc(C(F)(F)F)cn3)C3(c4ncccn4)CC3)ccc2nc1N] | 2-amino-3-methyl-N-(1-(2-pyrimidinyl)cyclopropyl)-N-((5-(trifluoromethyl)-2-pyridinyl)methyl)-6-quinolinecarboxamide | 479.2 |
| 431 | 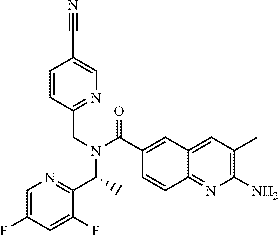 [SMILES: Cc1cc2cc(C(=O)N(Cc3ccc(C#N)cn3)[C@H](C)c3ncc(F)cc3F)ccc2nc1N] | 2-amino-N-((5-cyano-2-pyridinyl)methyl)-N-((1R)-1-(3,5-difluoro-2-pyridinyl)ethyl)-3-methyl-6-quinolinecarboxamide | 459.2 |
| 432 | 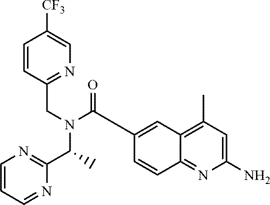 [SMILES: Cc1cc(N)nc2ccc(C(=O)N(Cc3ccc(C(F)(F)F)cn3)[C@H](C)c3ncccn3)cc12] | 2-amino-4-methyl-N-((1R)-1-(2-pyrimidinyl)ethyl)-N-((5-(trifluoromethyl)-2-pyridinyl)methyl)-6-quinolinecarboxamide | 467.2 |
| 433 | 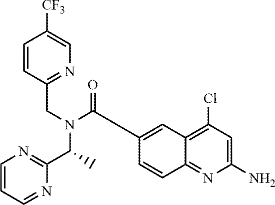 [SMILES: C[C@H](c1ncccn1)N(Cc1ccc(C(F)(F)F)cn1)C(=O)c1ccc2nc(N)cc(Cl)c2c1] | 2-amino-4-chloro-N-((1R)-1-(2-pyrimidinyl)ethyl)-N-((5-(trifluoromethyl)-2-pyridinyl)methyl)-6-quinolinecarboxamide | 487.3 |
| 434 | 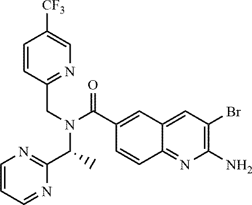 [SMILES: C[C@H](c1ncccn1)N(Cc1ccc(C(F)(F)F)cn1)C(=O)c1ccc2nc(N)c(Br)cc2c1] | 2-amino-3-bromo-N-((1R)-1-(2-pyrimidinyl)ethyl)-N-((5-(trifluoromethyl)-2-pyridinyl)methyl)-6-quinolinecarboxamide | 531/533 |
| 435 | 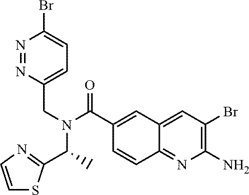 [SMILES: C[C@H](c1nccs1)N(Cc1ccc(Br)nn1)C(=O)c1ccc2nc(N)c(Br)cc2c1] | 2-amino-3-bromo-N-((6-bromo-3-pyridazinyl)methyl)-N-((1R)-1-(1,3-thiazol-2-yl)ethyl)-6-quinolinecarboxamide | 549 |
| 436 | 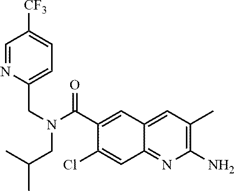 [SMILES: Cc1cc2cc(C(=O)N(Cc3ccc(C(F)(F)F)cn3)CC(C)C)c(Cl)cc2nc1N] | 2-amino-7-chloro-3-methyl-N-(2-methylpropyl)-N-((5-(trifluoromethyl)-2-pyridinyl)methyl)-6-quinolinecarboxamide | 451.1 |
| 437 | 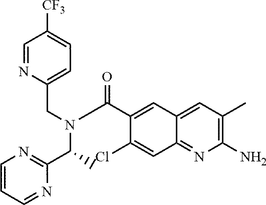 [SMILES: Cc1cc2cc(C(=O)N(Cc3ccc(C(F)(F)F)cn3)[C@H](C)c3ncccn3)c(Cl)cc2nc1N] | 2-amino-7-chloro-3-methyl-N-((1R)-1-(2-pyrimidinyl)ethyl)-N-((5-(trifluoromethyl)-2-pyridinyl)methyl)-6-quinolinecarboxamide | 501.1 |
| 438 | 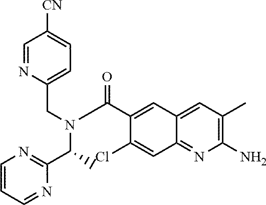 [SMILES: Cc1cc2cc(C(=O)N(Cc3ccc(C#N)cn3)[C@H](C)c3ncccn3)c(Cl)cc2nc1N] | 2-amino-7-chloro-N-((5-cyano-2-pyridinyl)methyl)-3-methyl-N-((1R)-1-(2-pyrimidinyl)ethyl)-6-quinolinecarboxamide | 458.1 |
| 439 | 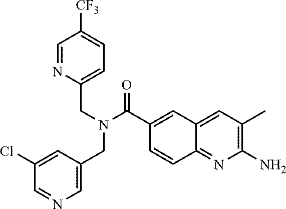 [SMILES: Cc1cc2cc(C(=O)N(Cc3cncc(Cl)c3)Cc3ccc(C(F)(F)F)cn3)ccc2nc1N] | 2-amino-N-((5-chloro-3-pyridinyl)methyl)-3-methyl-N-((5-(trifluoromethyl)-2-pyridinyl)methyl)-6-quinolinecarboxamide | 486.2 |
| 440 | 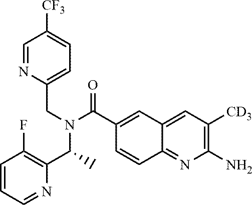 [SMILES: [2H]C([2H])([2H])c1cc2cc(C(=O)N(Cc3ccc(C(F)(F)F)cn3)[C@H](C)c3ncccc3F)ccc2nc1N] | (R)-2-amino-N-(1-(3-fluoropyridin-2-yl)ethyl)-3-(methyl-d₃)-N-((5- (trifluoromethyl)pyridin-2-yl)methyl)quinoline-6-carboxamide | 487.2 |
| 441 | 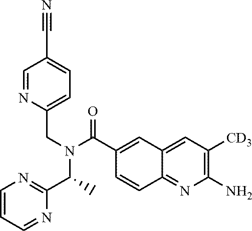 [SMILES: [2H]C([2H])([2H])c1cc2cc(C(=O)N(Cc3ccc(C#N)cn3)[C@H](C)c3ncccn3)ccc2nc1N] | (R)-2-amino-N-((5-cyanopyridin-2-yl)methyl)-3-(methyl-d₃)-N-(1-(pyrimidin-2- yl)ethyl)quinoline-6-carboxamide | 427.2 |
| 442 | 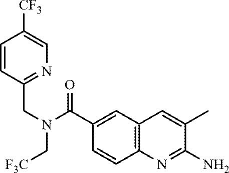 [SMILES: Cc1cc2cc(C(=O)N(Cc3ccc(C(F)(F)F)cn3)CC(F)(F)F)ccc2nc1N] | 2-amino-3-methyl-N-(2,2,2-trifluoroethyl)-N-((5-(trifluoromethyl)-2-pyridinyl)methyl)-6-quinolinecarboxamide | 443 |
| 728 | 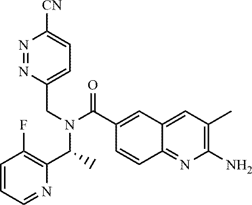 [SMILES: Cc1cc2cc(C(=O)N(Cc3ccc(C#N)nn3)[C@H](C)c3ncccc3F)ccc2nc1N] | 2-amino-N-((6-cyano-3-pyridazinyl)methyl)-N-((1R)-1-(3 -fluoro-2-pyridinyl)ethyl)-3 -methyl-6-quinolinecarboxamide | 442.2 |
| 729 | | 2-amino-3-bromo-N-((6-ethoxy-3-pyridazinyl)methyl)-N-((1R)-1-(2-pyrimidinyl)ethyl)-6-quinolinecarboxamide | 508.1, 510.0 |
| 730 | 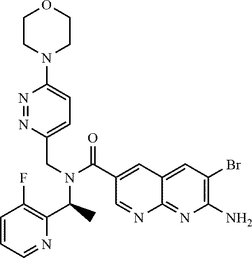 [SMILES: C[C@@H](c1ncccc1F)N(Cc1ccc(N2CCOCC2)nn1)C(=O)c1cnc2nc(N)c(Br)cc2c1] | 7-amino-6-bromo-N-((1R)-1-(3-fluoro-2-pyridinyl)ethyl)-N-((6-(4-morpholinyl)-3-pyridazinyl)methyl)-1,8-naphthyridine-3-carboxamide | 566.9, 568.8 |
| 731 | 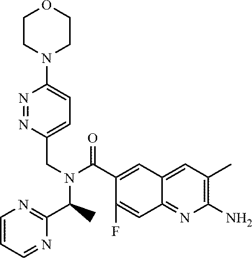 [SMILES: Cc1cc2cc(C(=O)N(Cc3ccc(N4CCOCC4)nn3)[C@@H](C)c3ncccn3)c(F)cc2nc1N] | 2-amino-7-fluoro-3-methyl-N-((6-(4-morpholinyl)-3-pyridazinyl)methyl)-N-((1R)-1-(2-pyrimidinyl)ethyl)-6-quinolinecarboxamide | 503.2 |
| 732 | 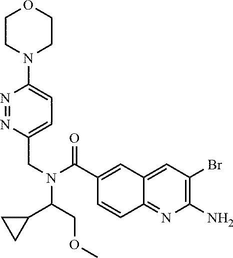 [SMILES: COCC(C1CC1)N(Cc1ccc(N2CCOCC2)nn1)C(=O)c1ccc2nc(N)c(Br)cc2c1] | 2-amino-3-bromo-N-((1S)-1-cyclopropyl-2-methoxyethyl)-N-((6-(4-morpholinyl)-3-pyridazinyl)methyl)-6-quinolinecarboxamide | 541.1, 543.3 |
| 733 | 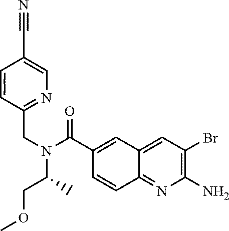 [SMILES: COC[C@@H](C)N(Cc1ccc(C#N)cn1)C(=O)c1ccc2nc(N)c(Br)cc2c1] | 2-amino-3-bromo-N-((5-cyano-2-pyridinyl)methyl)-N-((2R)-1-methoxy-2-propanyl)-6-quinolinecarboxamide | 454.1, 456.1 |
| 734 | 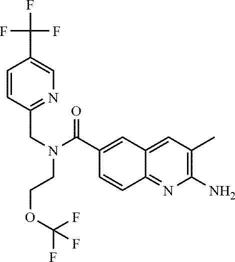 [SMILES: Cc1cc2cc(C(=O)N(CCOC(F)(F)F)Cc3ccc(C(F)(F)F)cn3)ccc2nc1N] | 2-amino-3-methyl-N-(2-(trifluoromethoxy)ethyl)-N-((5-(trifluoromethyl)-2-pyridinyl)methyl)-6-quinolinecarboxamide | 473.1 |
| 735 | 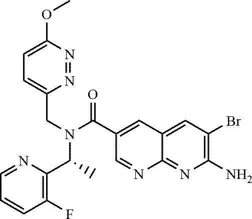 [SMILES: COc1ccc(CN(C(=O)c2cnc3nc(N)c(Br)cc3c2)[C@H](C)c2ncccc2F)nn1] | 7-amino-6-bromo-N-((1R)-1-(3-fluoro-2-pyridinyl)ethyl)-N-((6-methoxy-3-pyridazinyl)methyl)-1,8-naphthyridine-3-carboxamide | 512.2, 514.1 |
| 736 | 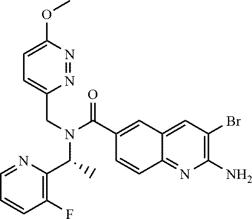 [SMILES: COc1ccc(CN(C(=O)c2ccc3nc(N)c(Br)cc3c2)[C@H](C)c2ncccc2F)nn1] | 2-amino-3-bromo-N-((1R)-1-(3-fluoro-2-pyridinyl)ethyl)-N-((6-methoxy-3-pyridazinyl)methyl)-6-quinolinecarboxamide | 511.1, 513.0 |
| 737 | 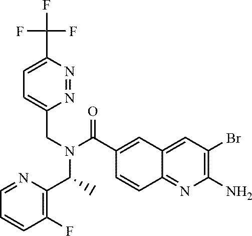 [SMILES: C[C@H](c1ncccc1F)N(Cc1ccc(C(F)(F)F)nn1)C(=O)c1ccc2nc(N)c(Br)cc2c1] | 2-amino-3-bromo-N-((1R)-1-(3-fluoro-2-pyridinyl)ethyl)-N-((6-(trifluoromethyl)-3-pyridazinyl)methyl)-6-quinolinecarboxamide | 549.0, 551.0 |
| 738 | 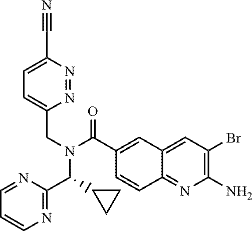 [SMILES: N#Cc1ccc(CN(C(=O)c2ccc3nc(N)c(Br)cc3c2)[C@@H](c2ncccn2)C2CC2)nn1] | 2-amino-3-bromo-N-((5-cyano-2-pyridinyl)methyl)-N-((R)-cyclopropyl(2-pyrimidinyl)methyl)-6-quinolinecarboxamide | 514.1, 516 |
| 739 | 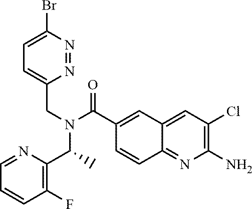 [SMILES: C[C@H](c1ncccc1F)N(Cc1ccc(Br)nn1)C(=O)c1ccc2nc(N)c(Cl)cc2c1] | 2-amino-N-((6-bromo-3-pyridazinyl)methyl)-3-chloro-N-((1R)-1-(3-fluoro-2-pyridinyl)ethyl)-6-quinolinecarboxamide | 515.0, 517.0 |
| 740 | 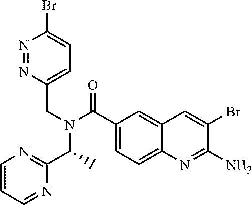 [SMILES: C[C@H](c1ncccn1)N(Cc1ccc(Br)nn1)C(=O)c1ccc2nc(N)c(Br)cc2c1] | (R)-2-amino-3-bromo-N-((6-bromopyridazin-3-yl)methyl)-N-(1-(pyrimidin-2-yl)ethyl)quinoline-6-carboxamide | 544.0 |
| 741 | 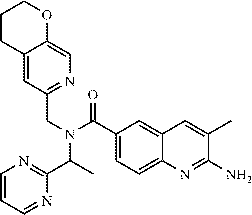 [SMILES: Cc1cc2cc(C(=O)N(Cc3cc4c(cn3)OCCC4)C(C)c3ncccn3)ccc2nc1N] | 2-amino-N-((3,4-dihydro-2H-pyrano[2,3-c]pyridin-6-yl)methyl)-3-methyl-N-(1-(pyrimidin-2-yl)ethyl)quinoline-6-carboxamide | 455.1 |
| 742 | 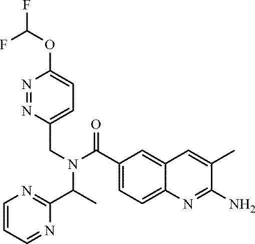 [SMILES: Cc1cc2cc(C(=O)N(Cc3ccc(OC(F)F)nn3)C(C)c3ncccn3)ccc2nc1N] | 2-amino-N-((6-(difluoromethoxy)pyridazin-3-yl)methyl)-3-methyl-N-(1-(pyrimidin-2-yl)ethyl)quinoline-6-carboxamide | 466.2 |
| 743 | 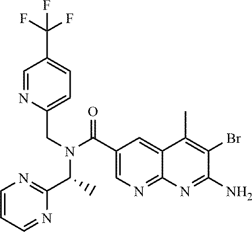 [SMILES: Cc1c(Br)c(N)nc2ncc(C(=O)N(Cc3ccc(C(F)(F)F)cn3)[C@H](C)c3ncccn3)cc12] | (R)-7-amino-6-bromo-5-methyl-N-(1-(pyrimidin-2-yl)ethyl)-N-((5-(trifluoromethyl)pyridin-2-yl)methyl)-1,8-naphthyridine-3-carboxamide | 546.2 |
| 744 | 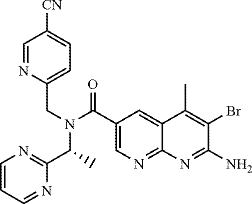 [SMILES: Cc1c(Br)c(N)nc2ncc(C(=O)N(Cc3ccc(C#N)cn3)[C@H](C)c3ncccn3)cc12] | (R)-7-amino-6-bromo-N-((5-cyanopyridin-2-yl)methyl)-5-methyl-N-(1-(pyrimidin-2-y 1)ethy 1)-1 ,8-naphthyridine-3-carboxamide | 503.1 |
| 745 | 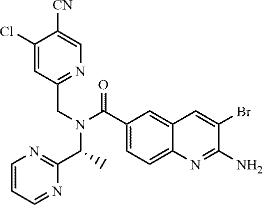 [SMILES: C[C@H](c1ncccn1)N(Cc1cc(Cl)c(C#N)cn1)C(=O)c1ccc2nc(N)c(Br)cc2c1] | (R)-2-amino-3-bromo-N-((4-chloro-5-cyanopyridin-2-yl)methyl)-N-(1-(pyrimidin-2-yl)ethyl)quinoline-6-carboxamide | 522.0 |
| 746 | | (R)-2-amino-3-bromo-N-((6-(difluoromethoxy)pyridazin-3-yl)methyl)-N-(1-(pyrimidin-2-yl)ethyl)quinoline-6-carboxamide | 530.0 |
| 747 | 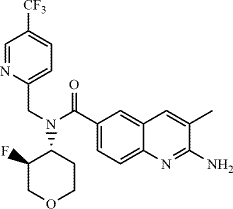 [SMILES: Cc1cc2cc(C(=O)N(Cc3ccc(C(F)(F)F)cn3)[C@@H]3CCOC[C@H]3F)ccc2nc1N] | 2-amino-N-((3R,4S)-3-fluorotetrahydro-2H-pyran-4-yl)-3-methyl-N-((5-(trifluoromethyl)-2-pyridinyl)methyl)-6-quinolinecarboxamide and 2-amino-N-((3S,4R)-3-fluorotetrahydro-2H-pyran-4-yl)-3-methyl-N-((5-(trifluoromethyl)-2-pyridinyl)methyl)-6-quinolinecarboxamide | 505.2 |
| 748 | 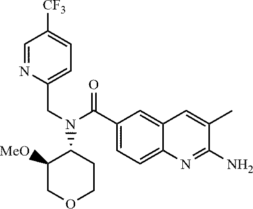 [SMILES: CO[C@@H]1COCC[C@H]1N(Cc1ccc(C(F)(F)F)cn1)C(=O)c1ccc2nc(N)c(C)cc2c1] | 2-amino-N-((3R,4S)-3-methoxytetrahydro-2H-pyran-4-yl)-3-methyl-N-((5-(trifluoromethyl)-2-pyridinyl)methyl)-6-quinolinecarboxamide and 2-amino-N-((3S,4R)-3-methoxytetrahydro-2H-pyran-4-yl)-3-methyl-N-((5-(trifluoromethyl)-2-pyridinyl)methyl)-6-quinolinecarboxamide | 475.0 |
| 749 | 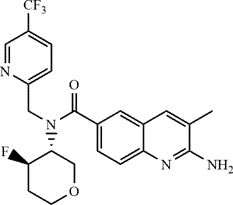 [SMILES: Cc1cc2cc(C(=O)N(Cc3ccc(C(F)(F)F)cn3)[C@@H]3COCC[C@H]3F)ccc2nc1N] | 2-amino-N-((3R,4R)-4-fluorotetrahydro-2H-pyran-3-yl)-3-methyl-N-((5-(trifluoromethyl)-2-pyridinyl)methyl)-6-quinolinecarboxamide and 2-amino-N-((3S,4S)-4-fluorotetrahydro-2H-pyran-3-yl)-3-methyl-N-((5-(trifluoromethyl)-2-pyridinyl)methyl)-6-quinolinecarboxamide | 463.2 |
| 750 | 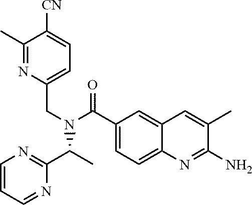 | 2-amino-N-((5-cyano-6-methyl-2-pyridinyl)methyl)-3-methyl-N-((1R)-1-(2-pyrimidinyl)ethyl)-6-quinolinecarboxamide | 438.2 |
| 751 | | 2-amino-N-((1R,2R)-2-methoxy-2,3-dihydro-1H-inden-1-yl)-3-methyl-N-((5-(trifluoromethyl)-2-pyridinyl)methyl)-6-quinolinecarboxamide | 507.2 |
| 752 | | 2-amino-N-((1S,2S)-2-methoxy-2,3-dihydro-1H-inden-1-yl)-3-methyl-N-((5-(trifluoromethyl)-2-pyridinyl)methyl)-6-quinolinecarboxamide | 507.2 |
| 753 | | 2-amino-N-((1S,2S)-2-methoxy-2,3-dihydro-1H-inden-1-yl)-3-methyl-N-((5-(trifluoromethyl)-2-pyridinyl)methyl)-6-quinolinecarboxamide | 433.3 |
| 754 | | N-((5-acetyl-2-pyridinyl)methyl)-2-amino-3-methyl-N-((1R)-1-(2-pyrimidinyl)ethyl)-6-quinolinecarboxamide | 441.2 |
| 755 | 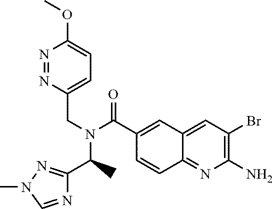 [SMILES: COc1ccc(CN(C(=O)c2ccc3nc(N)c(Br)cc3c2)[C@@H](C)c2ncn(C)n2)nn1] | (R)-2-amino-3-bromo-N-((6-methoxypyridazin-3-yl)methyl)-N-(1-(1-methyl-1H-1,2,4-triazol-3-yl)ethyl)quinoline-6-carboxamide | 497/499 |
| 756 | 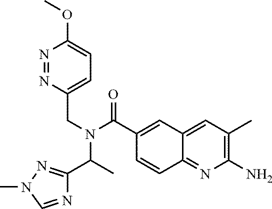 | 2-amino-N-((6-methoxypyridazin-3-yl)methyl)-3-methyl-N-(1-(1-methyl-1H-1,2,4-triazol-3-yl)ethyl)quinoline-6-carboxamide | 433 |
| 757 | 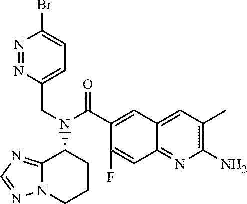 [SMILES: Cc1cc2cc(C(=O)N(Cc3ccc(Br)nn3)[C@@H]3CCCn4ncnc43)c(F)cc2nc1N] | (R)-2-amino-N-((6-bromopyridazin-3-yl)methyl)-7-fluoro-3-methyl-N-(5,6,7,8-tetrahydro-[1,2,4]triazolo[1,5-a]pyridin-8-yl)quinoline-6-carboxamide | 511/513 |
| 758 | 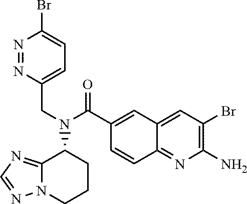 [SMILES: Nc1nc2ccc(C(=O)N(Cc3ccc(Br)nn3)[C@@H]3CCCn4ncnc43)cc2cc1Br] | (R)-2-amino-3-bromo-N-((6-bromopyridazin-3-yl)methyl)-N-(5,6,7,8-tetrahydro-[1,2,4]triazolo[1,5-a]pyridin-8-yl)quinoline-6-carboxamide | 559 |
| 759 | 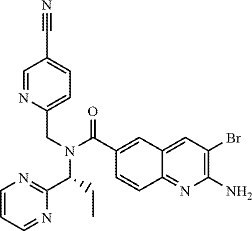 [SMILES: CC[C@H](c1ncccn1)N(Cc1ccc(C#N)cn1)C(=O)c1ccc2nc(N)c(Br)cc2c1] | (R)-2-amino-3-bromo-N-((5-cyanopyridin-2-yl)methyl)-N-(1-(pyrimidin-2-yl)propyl)quinoline-6-carboxamide | 502/504 |
| 760 | 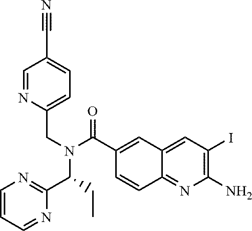 [SMILES: CC[C@H](c1ncccn1)N(Cc1ccc(C#N)cn1)C(=O)c1ccc2nc(N)c(I)cc2c1] | (R)-2-amino-N-((5-cyanopyridin-2-yl)methyl)-3-iodo-N-(1-(pyrimidin-2-yl)propyl)quinoline-6-carboxamide | 550 |
| 761 | 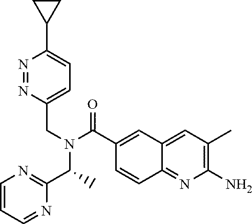 [SMILES: Cc1cc2cc(C(=O)N(Cc3ccc(C4CC4)nn3)[C@H](C)c3ncccn3)ccc2nc1N] | (R)-2-amino-N-((6-cyclopropylpyridazin-3-yl)methyl)-3-methyl-N-(1-(pyrimidin-2-yl)ethyl)quinoline-6-carboxamide | 440 |
| 762 | 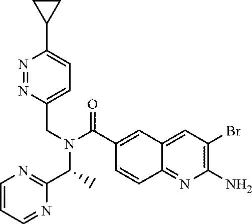 [SMILES: C[C@H](c1ncccn1)N(Cc1ccc(C2CC2)nn1)C(=O)c1ccc2nc(N)c(Br)cc2c1] | (R)-2-amino-3-bromo-N-((6-cyclopropylpyridazin-3-yl)methyl)-N-(1-(pyrimidin-2-yl)ethyl)quinoline-6-carboxamide | 504/506 |
| 763 | 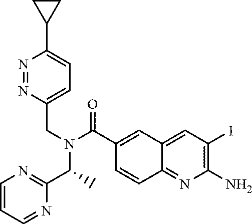 [SMILES: C[C@H](c1ncccn1)N(Cc1ccc(C2CC2)nn1)C(=O)c1ccc2nc(N)c(I)cc2c1] | (R)-2-amino-N-((6-cyclopropylpyridazin-3-yl)methyl)-3-iodo-N-(1-(pyrimidin-2-yl)ethyl)quinoline-6-carboxamide | 552 |
| 764 | 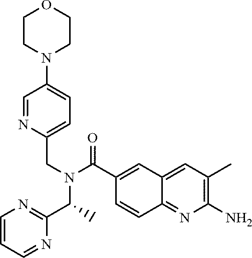 [SMILES: Cc1cc2cc(C(=O)N(Cc3ccc(N4CCOCC4)cn3)[C@H](C)c3ncccn3)ccc2nc1N] | (R)-2-amino-3-methyl-N-((5-morpholinopyridin-2-yl)methyl)-N-(1-(pyrimidin-2-yl)ethyl)quinoline-6-carboxamide | 484 |
| 765 | 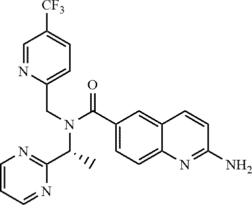 [SMILES: C[C@H](c1ncccn1)N(Cc1ccc(C(F)(F)F)cn1)C(=O)c1ccc2nc(N)ccc2c1] | (R)-2-amino-N-(1-(pyrimidin-2-yl)ethyl)-N-((5-(trifluoromethyl)pyridin-2-yl)methyl)quinoline-6-carboxamide | 453.2 |
| 766 | 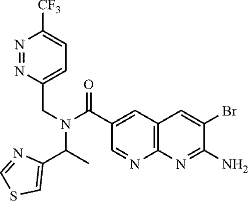 [SMILES: CC(c1cscn1)N(Cc1ccc(C(F)(F)F)nn1)C(=O)c1cnc2nc(N)c(Br)cc2c1] | 7-amino-6-bromo-N-(1 -(thiazol-4-yl)ethyl)-N-((6-(trifluoromethyl)pyridazin-3-yl)methyl)-1, 8-naphthyridine-3 -carboxamide | 538/540 |
| 767 | 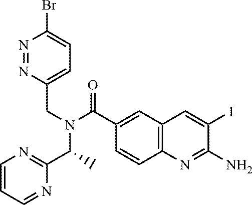 [SMILES: C[C@H](c1ncccn1)N(Cc1ccc(Br)nn1)C(=O)c1ccc2nc(N)c(I)cc2c1] | (R)-2-amino-N-((6-bromopyridazin-3-yl)methyl)-3-iodo-N-(1-(pyrimidin-2-yl)ethyl)quinoline-6-carboxamide | 590/592 |
| 768 | 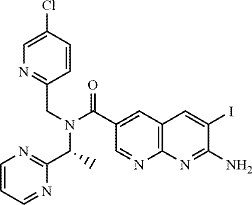 [SMILES: C[C@H](c1ncccn1)N(Cc1ccc(Cl)cn1)C(=O)c1cnc2nc(N)c(I)cc2c1] | (R)-7-amino-N-((5-chloropyridin-2-yl)methyl)-6-iodo-N-(1-(pyrimidin-2-yl)ethyl)-1,8-naphthyridine-3-carboxamide | 545.9 |
| 769 | 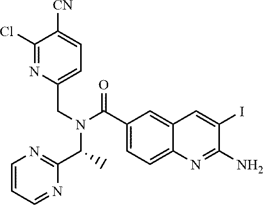 [SMILES: C[C@H](c1ncccn1)N(Cc1ccc(C#N)c(Cl)n1)C(=O)c1ccc2nc(N)c(I)cc2c1] | (R)-2-amino-N-((6-chloro-5-cyanopyridin-2-yl)methyl)-3-iodo-N-(1-(pyrimidin-2-yl)ethyl)quinoline-6-carboxamide | 570 |
| 770 | 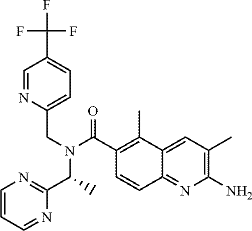 [SMILES: Cc1cc2c(C)c(C(=O)N(Cc3ccc(C(F)(F)F)cn3)[C@H](C)c3ncccn3)ccc2nc1N] | 2-amino-3,5-dimethyl-N-((1R)-1-(2-pyrimidinyl)ethyl)-N-((5-(trifluoromethyl)-2-pyridinyl)methyl)-6-quinolinecarboxamide | 480.5 |
| 771 | 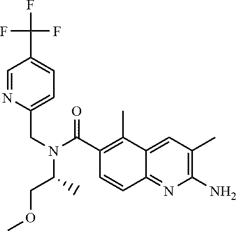 [SMILES: COC[C@@H](C)N(Cc1ccc(C(F)(F)F)cn1)C(=O)c1ccc2nc(N)c(C)cc2c1C] | 2-amino-N-((2R)-1-methoxy-2-propanyl)-3,5-dimethyl-N-((5-(trifluoromethyl)-2-pyridinyl)methyl)-6-quinolinecarboxamide | 446.5 |
| 772 | 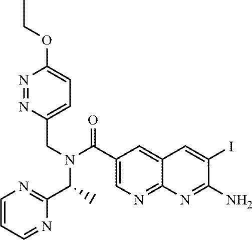 [SMILES: CCOc1ccc(CN(C(=O)c2cnc3nc(N)c(I)cc3c2)[C@H](C)c2ncccn2)nn1] | (R)-7-amino-N-((6-ethoxypyridazin-3-yl)methyl)-6-iodo-N-(1-(pyrimidin-2-yl)ethyl)-1,8-naphthyridine-3-carboxamide | 557.2 |
| 773 | 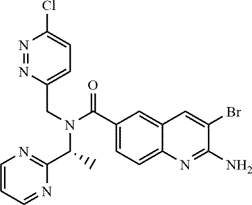 [SMILES: C[C@H](c1ncccn1)N(Cc1ccc(Cl)nn1)C(=O)c1ccc2nc(N)c(Br)cc2c1] | (R)-2-amino-3-bromo-N-((6-chloropyridazin-3-yl)methyl)-N-(1-(pyrimidin-2-yl)ethyl)quinoline-6-carboxamide | 498, 500 |

Example 443: (R)-7-amino-6-bromo-N-(1-(pyrimidin-2-yl)propyl)-N-((5-(trifluoromethyl)pyridin-2-yl)methyl)-1,8-naphthyridine-3-carboxamide. (Method A-PyBroP-SFC)

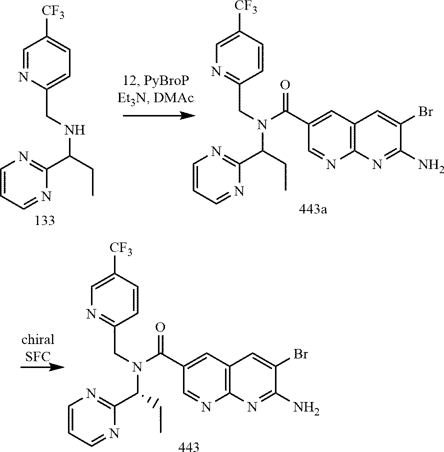

Bromotripyrrolidinophosphonium hexafluorophosphate (PyBroP) (413 mg, 0.88 mmol) was added to a mixture of 7-amino-6-bromo-1,8-naphthyridine-3-carboxylic acid (12, 249 mg, 0.93 mmol), 1-(pyrimidin-2-yl)-N-((5-(trifluoromethyl)pyridin-2-yl)methyl)propan-1-amine (133, 250 mg, 0.84 mmol) and triethylamine (0.35 mL, 2.53 mmol) in 2 mL of DMAc at RT. The mixture was stirred at RT for 24 h then partitioned between 50 mL EtOAc and 5 mL of water. The layers were separated. The organic layer was washed with 5 mL of 1 N NaOH and concentrated. The residue was purified on a silica gel column (50% EtOAc in DCM followed by 5-8% MeOH in DCM)to give a brown solid as a mixture of 7-amino-6-bromo-N-(1-(pyrimidin-2-yl)propyl)-N-((5-(trifluoromethyl)pyridin-2-yl)methyl)-1,8-naphthyridine-3-carboxamide (443a, m/z (ESI): 546/548 (M+H)⁺) and tri(pyrrolidin-1-yl)phosphine oxide (m/z (ESI): 258.1 (M/H)⁺).

The racemic material was subjected to chiral SFC under the conditions indicated below to give 2 eluents. Chiral SFC: column Chiralcel OD-H SFC, 5 µm, 21 × 250 mm column, with a modifier of 20% EtOH (with 0.2% TEA) using a flowrate of 80 mL/min. The more potent (measured by IC₅₀ in HCT116 MTAP null cell viability assay) enantiomer was assigned as the (R)-; the less potent (measured by IC₅₀ in HCT116 MTAP null cell viability assay) enantiomer was assigned as (S)-. The 1^{st} eluting peak was (R)-7-amino-6-bromo-N-(1-(pyrimidin-2-yl)propyl)-N-((5-(trifluoromethyl)pyridin-2-yl)methyl)-1,8-naphthyridine-3-carboxamide (443, 82 mg). The 2^{nd} eluting peak was (S)-7-amino-6-bromo-N-(1-(pyrimidin-2-yl)propyl)-N-((5-(trifluoromethyl)pyridin-2-yl)methyl)-1,8-naphthyridine-3-carboxamide (55 mg).m/z (ESI): 546/548 (M+H)⁺). ¹H NMR (METHANOL-d₄, 400 MHz) δ9.10 (s, 1H), 8.73 (d, 2H, J=4.4 Hz), 8.67 (s, 1H), 8.5-8.6 (m, 2H), 7.9-8.0 (m, 1H), 7.4-7.5 (m, 1H), 7.32 (t, 1H, J=4.9 Hz), 5.25 (m, 1H), 5.05 (m, 1H), 4.94 (m, 1H), 2.27 (m, 2H), 1.25 (m, 3H).

TABLE 10

Examples below were prepared in a manner similar to that described above (Method A-PyBroP-SFC) for Example 443

| Ex. # | Chemical Structure | Name | Peak sequence and SFC conditions | LRMS: (ESI+) m/z |
|---|---|---|---|---|
| 444 | 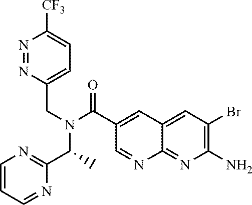 [SMILES: C[C@H](c1ncccn1)N(Cc1ccc(C(F)(F)F)nn1)C(=O)c1cnc2nc(N)c(Br)cc2c1] | 7-amino-6-bromo-N-((1R)-1-(2-pyrimidinyl)ethyl)-N-((6-(trifluoromethyl)-3-pyridazinyl)methyl)-1,8-naphthyridine-3-carboxamide | 1st Peak. Chiral Technologies IA (250 × 21 mm, 5 µm). Mobile phase: 65:35. B: 0.2% TEA in EtOH. | 533/535 |
| 445 | 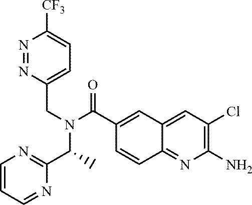 [SMILES: C[C@H](c1ncccn1)N(Cc1ccc(C(F)(F)F)nn1)C(=O)c1ccc2nc(N)c(Cl)cc2c1] | 2-amino-3-chloro-N-((1R)-1-(2-pyrimidinyl)ethyl)-N-((6-(trifluoromethyl)-3 -pyridazinyl)methyl)-6-quinolinecarboxamide | 1st Peak. Chiral Technologies AD (250 × 21 mm, 5 µm). Mobile phase: 60:40. B: 0.2% TEA in EtOH. | 488 |
| 446 | 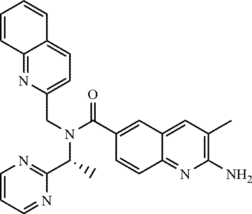 [SMILES: Cc1cc2cc(C(=O)N(Cc3ccc4ccccc4n3)[C@H](C)c3ncccn3)ccc2nc1N] | 2-amino-3-methyl-N-((1R)-1-(2-pyrimidinyl)ethyl)-N-(2-quinolinylmethyl)-6-quinolinecarboxamide | 2nd Peak. Chiral Technologies OJ (250 × 21 mm, 5 µm). Mobile phase: 80:20. B: 0.2% TEA in EtOH. | 449 |
| 447 | 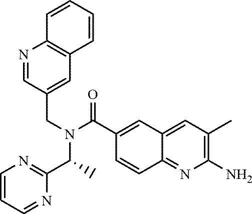 [SMILES: Cc1cc2cc(C(=O)N(Cc3cnc4ccccc4c3)[C@H](C)c3ncccn3)ccc2nc1N] | 2-amino-3-methyl-N-((1R)-1-(2-pyrimidinyl)ethyl)-N-(3-quinolinylmethyl)-6-quinolinecarboxamide | 1st Peak. Chiral Technologies AD (250 × 21 mm, 5 µm). Mobile phase: 60:40. B: 0.2% TEA in EtOH. | 449 |
| 448 | 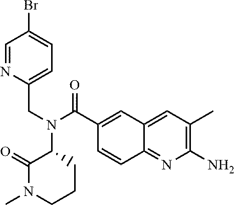 [SMILES: Cc1cc2cc(C(=O)N(Cc3ccc(Br)cn3)[C@@H]3CCCN(C)C3=O)ccc2nc1N] | 2-amino-N-((5-bromo-2-pyridinyl)methyl)-3- methyl-N-((3R)-1-methyl-2-oxo-3-piperidinyl)-6-quinolinecarboxamide | 1st Peak. Regis Whelk-O S,S (2×25 cm, 5 µm). Mobile phase: 40:60. B: 0.2% DEA in MeOH. | 482/484 |
| 449 | 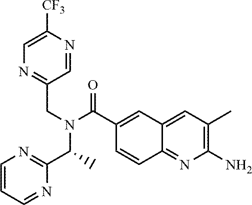 [SMILES: Cc1cc2cc(C(=O)N(Cc3cnc(C(F)(F)F)cn3)[C@H](C)c3ncccn3)ccc2nc1N] | 2-amino-3-methyl-N-((1R)-1-(2-pyrimidinyl)ethyl)-N-((5-(trifluoromethyl)-2-pyrazinyl)methyl)-6-quinolinecarboxamide | Peak 1. Whelk-O S,S (30 × 150 mm, 5 µm); Mobile phase: 60:40; B: 0.2% DEA in (1:1 MeOH:DCM). | 468 |
| 450 | 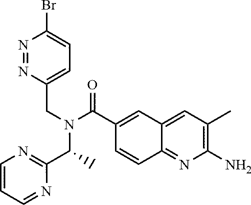 [SMILES: Cc1cc2cc(C(=O)N(Cc3ccc(Br)nn3)[C@H](C)c3ncccn3)ccc2nc1N] | 2-amino-N-((6-bromo-3-pyridazinyl)methyl)-3-methy-N-((1R)-1-(2-pyrimidinyl)ethyl)-6-quinolinecarboxamide | Peak 2. Regis (R,R) Whelk-01 (250 × 21 mm, 5 µm). Mobile phase: 55:45. B: MeOH. | 478.1/48 0.1 |
| 451 | 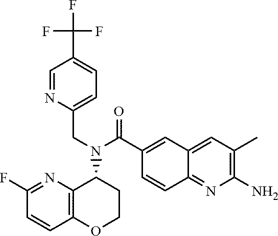 [SMILES: Cc1cc2cc(C(=O)N(Cc3ccc(C(F)(F)F)cn3)[C@@H]3CCOc4ccc(F)nc43)ccc2nc1N] | 2-amino-N-((4R)-6-fluoro-3,4-dihydro-2H-pyrano [3,2-b] pyridin-4-yl)-3-methyl-N-((5-(trifluoromethyl)-2-pyridinyl)methyl)-6-quinolinecarboxamide | 1st peak. Chiral Tech AS column (150 × 30 mm, 5 µm. Mobile phase: 75:25. B: 0.2% TEA in MeOH. | 512.2 |
| 452 | 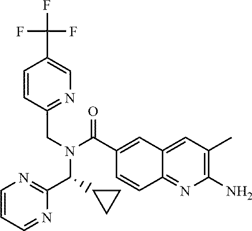 [SMILES: Cc1cc2cc(C(=O)N(Cc3ccc(C(F)(F)F)cn3)[C@@H](c3ncccn3)C3CC3)ccc2nc1N] | 2-amino-N-((R)-cyclopropyl(2-pyrimidinyl)methyl)-3-methyl-N-((5-(trifluoromethyl)-2-pyridinyl)methyl)-6-quinolinecarboxamide | 2nd Peak. Chiral Technologies OZ column (250 × 21 mm, 5 µm). Mobile phase: 65:35. B: 0.2% TEA in EtOH. | 493 |
| 453 | 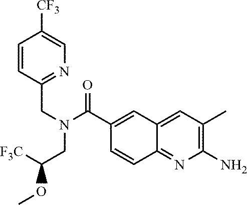 [SMILES: CO[C@H](CN(Cc1ccc(C(F)(F)F)cn1)C(=O)c1ccc2nc(N)c(C)cc2c1)C(F)(F)F] | 2-amino-3-methyl-N-((2R)-3,3,3 -trifluoro-2-methoxypropyl)-N-((5-(trifluoromethyl)-2-pyridinyl)methyl)-6-quinolinecarboxamide | 2nd Peak. Chiral Technologies AD column (250 × 21 mm, 5 µm); mobile phase: 90:10. B: 0.2% TEA in EtOH. | 487.1 |
| 454 | 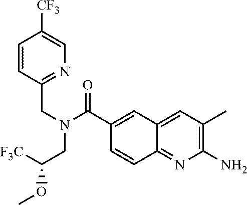 [SMILES: CO[C@@H](CN(Cc1ccc(C(F)(F)F)cn1)C(=O)c1ccc2nc(N)c(C)cc2c1)C(F)(F)F] | 7-amino-6-bromo-N-((2S)-3,3,3-trifluoro-2-methoxypropyl)-N-((5-(trifluoromethyl)-2-pyridinyl)methyl)-1,8-naphthyridine-3-carboxamide | 1st Peak. Chiral Technologies OX column (250 × 21 mm, 5 µm). Mobile phase: 80:20. B: 0.2% TEA in MeOH> | 552 |
| 455 | 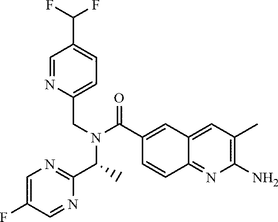 [SMILES: Cc1cc2cc(C(=O)N(Cc3ccc(C(F)F)cn3)[C@H](C)c3ncc(F)cn3)ccc2nc1N] | 2-amino-N-((5-(difluoromethyl)-2-pyridinyl)methyl)-N-((1R)-1-(5-fluoro-2-pyrimidinyl)ethyl)-3-methyl-6-quinolinecarboxamide | 1st Peak. Regis Whelk-O S,S 2×25 cm, 5 µm; mobile phase of 50% MeOH w/ 0.2% DEA | 467.1 |
| 456 | 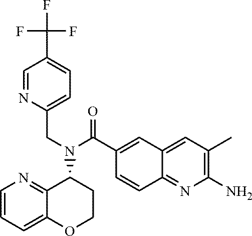 [SMILES: Cc1cc2cc(C(=O)N(Cc3ccc(C(F)(F)F)cn3)[C@@H]3CCOc4cccnc43)ccc2nc1N] | 2-amino-N-((4R)-3,4-dihydro-2H-pyrano [3,2-b] pyridin-4-yl)-3-methyl-N-((5-(trifluoromethyl)-2-pyridinyl)methyl)-6-quinolinecarboxamide | 1st Peak. Chiral Technologies AS (150 × 21 mm, 5 µm). Mobile phase: 80:20. B: 0.2% TEA in MeOH. | 494.2 |
| 457 | 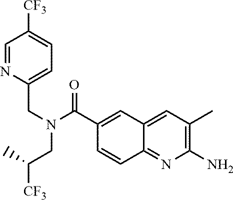 [SMILES: Cc1cc2cc(C(=O)N(Cc3ccc(C(F)(F)F)cn3)C[C@@H](C)C(F)(F)F)ccc2nc1N] | 2-amino-3-methyl-N-((2R)-3,3,3 -trifluoro-2-methylpropyl)-N-((5-(trifluoromethyl)-2-pyridinyl)methyl)-6-quinolinecarboxamide | 1st Peak. Chiral Technologies AD (250 × 21 mm, 5 mm) and AD (150 × 21 mm, 5 µm). Mobile phase: 65;35. B: 0.2% TEA in iPrOH | 471.2 |
| 458 | 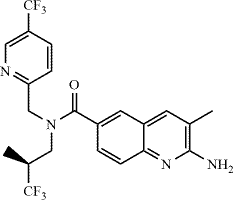 [SMILES: Cc1cc2cc(C(=O)N(Cc3ccc(C(F)(F)F)cn3)C[C@H](C)C(F)(F)F)ccc2nc1N] | 2-amino-3-methyl-N-((2S)-3,3,3-trifluoro-2-methylpropyl)-N-((5-(trifluoromethyl)-2-pyridinyl)methyl)-6-quinolinecarboxamide | 2 nd Peak. Chiral Technologies AD (250 × 21 mm, 5 mm) and AD (150 × 21 mm, 5 µm). Mobile phase: 65;35. B: 0.2% TEA in iPrOH | 471.2 |
| 459 | 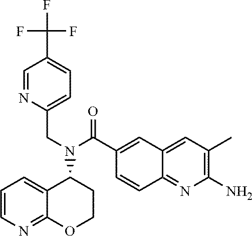 [SMILES: Cc1cc2cc(C(=O)N(Cc3ccc(C(F)(F)F)cn3)[C@@H]3CCOc4ncccc43)ccc2nc1N] | 2-amino-N-((4R)-3,4-dihydro-2H-pyrano [2,3 -b] pyridin-4-yl)-3-methyl-N-((5-(trifluoromethyl)-2-pyridinyl)methyl)-6-quinolinecarboxamide | 1st Peak. Chiral Technologies AD (250 × 21 mm, 5 µm). Mobile phase: 70:30. B: 0.2% TEA in iPrOH. | 494.1 |
| 460 | 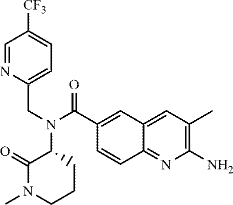 [SMILES: Cc1cc2cc(C(=O)N(Cc3ccc(C(F)(F)F)cn3)[C@@H]3CCCN(C)C3=O)ccc2nc1N] | 2-amino-3-methyl-N-((3R)-1-methyl-2-oxo-3-piperidinyl)-N-((5-(trifluoromethyl)-2-pyridinyl)methyl)-6-quinolinecarboxamide | 1st Peak. Chiral Technologies OX (250 × 21 mm, 5 µm). Mobile phase: 60:40. B: 0.2% TEA in EtOH. | 472.3 |
| 461 | 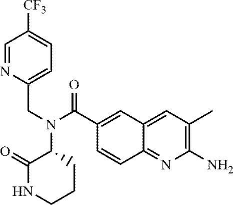 [SMILES: Cc1cc2cc(C(=O)N(Cc3ccc(C(F)(F)F)cn3)[C@@H]3CCCNC3=O)ccc2nc1N] | 2-amino-3-methyl-N-((3R)-2-oxo-3-piperidinyl)-N-((5-(trifluoromethyl)-2-pyridinyl)methyl)-6-quinolinecarboxamide | 1st Peak. Chiral Technologies AS (250 × 21 mm, 5 µm). Mobile phase: 75:25. B: 0.2% TEA in EtOH. | 458.1 |
| 462 | 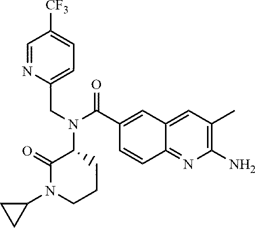 [SMILES: Cc1cc2cc(C(=O)N(Cc3ccc(C(F)(F)F)cn3)[C@@H]3CCCN(C4CC4)C3=O)ccc2nc1N] | 2-amino-N-((3R)-1-cyclopropyl-2-oxo-3-piperidinyl)-3 -methyl-N-((5-(trifluoromethyl)-2-pyridinyl)methyl)-6-quinolinecarboxamide | 2nd Peak. Regis (R,R) Whelk-01 (250 × 21 mm, 5 µm). Mobile phase: 60:40. B: 0.2% TEA in EtOH. | 498.2 |
| 463 | 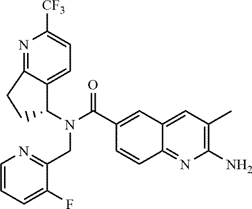 [SMILES: Cc1cc2cc(C(=O)N(Cc3ncccc3F)[C@@H]3CCc4nc(C(F)(F)F)ccc43)ccc2nc1N] | 2-amino-N-((3-fluoro-2-pyridinyl)methyl)-3 -methyl-N-((5R)-2-(trifluoromethy l) -6,7-dihydro-5H-cyclopenta[b]pyridin-5-yl)-6-quinolinecarboxamide | 1st peak, Chiral Technologies OJ column (250 × 21 mm, 5 mm) with a mobile phase of 85% liquid CO₂ and 15% EtOH with 0.2% TEA | 496.3 |
| 464 | 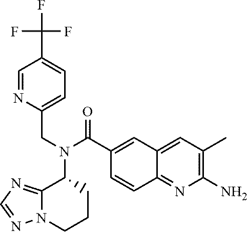 [SMILES: Cc1cc2cc(C(=O)N(Cc3ccc(C(F)(F)F)cn3)[C@@H]3CCCn4ncnc43)ccc2nc1N] | 2-amino-3-methyl-N-((8R)-5,6,7,8-tetrahydro [ 1,2,4] triazol o[1,5-a]pyridin-8-yl)-N-((5-(trifluoromethyl)-2-pyridinyl)methyl)-6-quinolinecarboxamide | 1st peak, Chiral Technologies IA column (250 × 21 mm, 5 µm) with a mobile phase of 73% liquid CO₂ and 27% EtOH with 0.2% TEA | 482.2 |
| 465 | 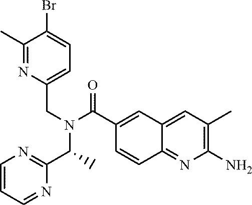 [SMILES: Cc1cc2cc(C(=O)N(Cc3ccc(Br)c(C)n3)[C@H](C)c3ncccn3)ccc2nc1N] | 2-amino-N-((5 -bromo-6-methyl-2-pyridinyl)methyl)-3-methyl-N-((1R)-1-(2-pyrimidinyl)ethyl)-6-quinolinecarboxamide | 1st peak, Chiral Technologies OJ column (250 ×21 mm, 5 µm) with a mobile phase of 85% liquid CO₂ and 15% EtOH with 0.2% TEA | 491.1/49 3.1 |
| 466 | 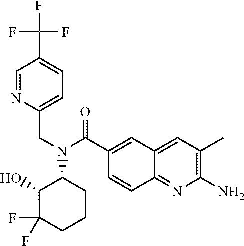 [SMILES: Cc1cc2cc(C(=O)N(Cc3ccc(C(F)(F)F)cn3)[C@@H]3CCCC(F)(F)[C@@H]3O)ccc2nc1N] | 2-amino-N-((1R,2R)-3,3-difluoro-2-hydroxycyclohexyl)-3-methyl-N-((5-(trifluoromethyl)-2-pyridinyl)methyl)-6-quinolinecarboxamide | 2nd peak, SFC via an Regis Whelk-O S,S 2 × 25 cm, 5 µm column; mobile phase of 40% MeOH w/ 0.2% DEA | 495.2 |
| 467 | 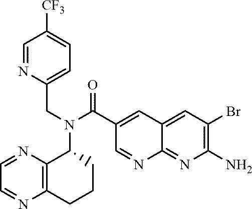 [SMILES: Nc1nc2ncc(C(=O)N(Cc3ccc(C(F)(F)F)cn3)[C@@H]3CCCc4nccnc43)cc2cc1Br] | 7-amino-6-bromo-N-((5R)-5,6,7,8-tetrahydro-5-quinoxalinyl)-N-((5-(trifluoromethyl)-2-pyridinyl)methyl)-1,8-naphthyridine-3-carboxamide | 1st peak, SFC using a Chiral Technologies OX column (250 × 21 mm, 5 µm) with a mobile phase of 65% liquid CO₂ and 35% MeOH with 0.2% TEA | 558/560 |
| 468 | 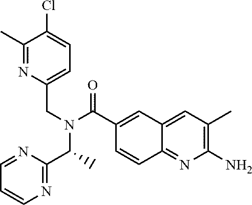 [SMILES: Cc1cc2cc(C(=O)N(Cc3ccc(Cl)c(C)n3)[C@H](C)c3ncccn3)ccc2nc1N] | 2-amino-N-((5-chloro-6-methyl-2-pyridinyl)methyl)-3-methyl-N-((1R)-1-(2-pyrimidinyl)ethyl)-6-quinolinecarboxamide | 1st peak, Chiral Technologies AD column (250 × 21 mm, 5 µm) with a mobile phase of 75% liquid CO₂ and 25% iPrOH with 0.2% TEA | 447.2 |
| 469 | 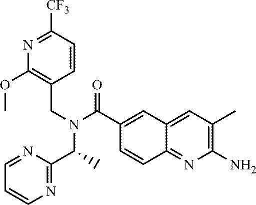 [SMILES: COc1nc(C(F)(F)F)ccc1CN(C(=O)c1ccc2nc(N)c(C)cc2c1)[C@H](C)c1ncccn1] | 2-amino-N-((2-methoxy-6-(trifluoromethyl) -3 -pyridinyl)methyl)-3-methyl-N-((1R)-1-(2-pyrimidinyl)ethyl)-6-quinolinecarboxamide | 1st peak, Chiral Technologies AS column (250 × 21 mm, 5 µm) with a mobile phase of 80% liquid CO₂ and 20% MeOH with 0.2% TEA | 497.2 |
| 470 | 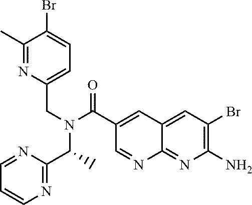 [SMILES: Cc1nc(CN(C(=O)c2cnc3nc(N)c(Br)cc3c2)[C@H](C)c2ncccn2)ccc1Br] | 7-amino-6-bromo-N-((5-bromo-6-methyl-2-pyridinyl)methyl)-N-((1R)-1-(2-pyrimidinyl)ethyl)-1,8-naphthyridine-3-carboxamide | 1st peak, Chiral Technologies AD column (250 × 21 mm, 5 µm) with a mobile phase of 60% liquid CO₂ and 40% iPrOH with 0.2% TEA | 558 |
| 471 | 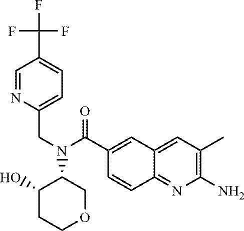 [SMILES: Cc1cc2cc(C(=O)N(Cc3ccc(C(F)(F)F)cn3)[C@@H]3COCC[C@@H]3O)ccc2nc1N] | 2-amino-N-((3R,4S)-4-hydroxytetrahydro-2H-pyran-3-yl)-3-methyl-N-((5-(trifluoromethyl)-2-pyridinyl)methyl)-6-quinolinecarboxamide | 2nd peak, Chiral Technologies AS column (250 × 21 mm, 5 µm) with a mobile phase of 85% liquid CO₂ and 15% MeOH with 0.2% TEA | 461.2 |
| 472 | 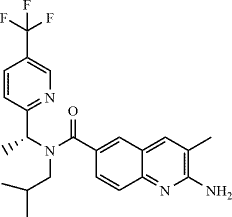 [SMILES: Cc1cc2cc(C(=O)N(CC(C)C)[C@H](C)c3ccc(C(F)(F)F)cn3)ccc2nc1N] | 2-amino-3-methyl-N-(2-methylpropyl)-N-((1R)-1-(5-(trifluoromethyl)-2-pyridinyl)ethyl)-6-quinolinecarboxamide | Peak 2. Regis Whelk-O S,S (2 × 25 cm, 5 µm). Mobile phase: 60:40. B: 0.2% DEA in MeOH. | 431 |
| 473 | 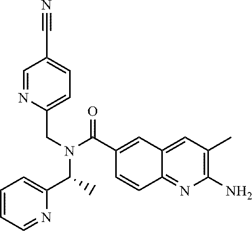 [SMILES: Cc1cc2cc(C(=O)N(Cc3ccc(C#N)cn3)[C@H](C)c3ccccn3)ccc2nc1N] | 2-amino-N-((5-cyano-2-pyridinyl)methyl)-3 -methyl-N-((1R)-1-(2-pyridinyl)ethyl)-6-quinolinecarboxamide | Peak 1. Chiralpak AD-H column (2 × 15 cm, 5 µm. Mobile phase: 50:50. B: 0.2% DEA in MeOH. | 423 |
| 474 | 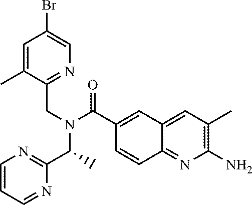 | 2-amino-N-((5 -bromo-3-methyl-2-pyridinyl)methyl)-3-methyl-N-((1R)-1-(2-pyrimidinyl)ethyl)-6-quinolinecarboxamide | Peak 1. Chiral Technologies IA column(250 × 21 mm, 5 µm). Mobile phase: 65:35. B: 0.2% TEA in EtOH. | 491/493 |
| 475 | 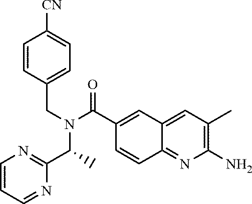 [SMILES: Cc1cc2cc(C(=O)N(Cc3ccc(C#N)cc3)[C@H](C)c3ncccn3)ccc2nc1N] | 2-amino-N-(4-cyanobenzyl)-3-methyl-N-((1R)-1-(2-pyrimidinyl)ethyl)-6-quinolinecarboxamide | Peak 1. Chiral Technologies OJ column (250 × 21 mm, 5 µm). Mobile phase: 82:18. B: 0.2% TEA in EtOH. | 423 |
| 476 | 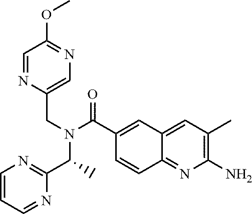 [SMILES: COc1cnc(CN(C(=O)c2ccc3nc(N)c(C)cc3c2)[C@H](C)c2ncccn2)cn1] | 2-amino-N-((5-methoxy-2-pyrazinyl)methyl)-3 -methyl-N-((1R)-1-(2-pyrimidinyl)ethyl)-6-quinolinecarboxamide | Peak 1. Chiral Technologies AD column (250 × 21 mm, 5 µm). Mobile phase: 70:30. B: 0.2% TEA in EtOH. | 430 |
| 477 | 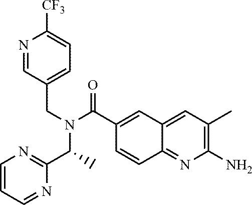 [SMILES: Cc1cc2cc(C(=O)N(Cc3ccc(C(F)(F)F)nc3)[C@H](C)c3ncccn3)ccc2nc1N] | 2-amino-3-methyl-N-((1R)-1-(2-pyrimidinyl)ethyl)-N-((6-(trifluoromethyl)-3 -pyridinyl)methyl)-6-quinolinecarboxamide | Peak 1. Chiral Technologies AD column (250 × 21 mm, 5 µm). Mobile phase: 75:25. B: 0.2% TEA in EtOH. | 467 |
| 478 | 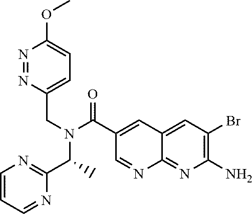 [SMILES: COc1ccc(CN(C(=O)c2cnc3nc(N)c(Br)cc3c2)[C@H](C)c2ncccn2)nn1] | 7-amino-6-bromo-N-((6-methoxy-3-pyridazinyl)methyl)-N-((1R)-1-(2-pyrimidinyl)ethyl)-1,8-naphthyridine-3-carboxamide | Peak 1. Chiralcel OD-H SFC, 5 µm, 21 × 250 mm column. Mobile phase: 65:35. B: 0.2% TEA in MeOH. | 495/497 |
| 479 | 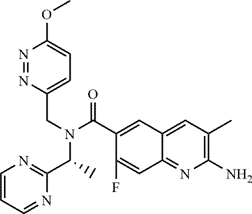 [SMILES: COc1ccc(CN(C(=O)c2cc3cc(C)c(N)nc3cc2F)[C@H](C)c2ncccn2)nn1] | 2-amino-7-fluoro-N-((6-methoxy-3-pyridazinyl)methyl)-3 -methyl-N-((1R)-1-(2-pyrimidinyl)ethyl)-6-quinolinecarboxamide | Peak 1. Chiralcel OD-H SFC, 5 µm, 21 × 250 mm column. Mobile phase: 60:40. B: 0.2% TEA in EtOH. | 448 |
| 480 | 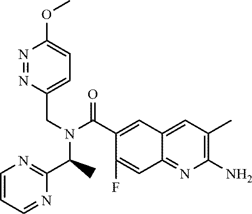 [SMILES: COc1ccc(CN(C(=O)c2cc3cc(C)c(N)nc3cc2F)[C@@H](C)c2ncccn2)nn1] | 2-amino-7-fluoro-N-((6-methoxy-3-pyridazinyl)methyl)-3 -methyl-N-((1S)-1-(2-pyrimidinyl)ethyl)-6-quinolinecarboxamide | Peak 2. Chiralcel AD-H SFC, 5 µm, 21 × 250 mm. Mobile phase: 60:40. B: 0.2% TEA in EtOH. | 448 |
| 481 | 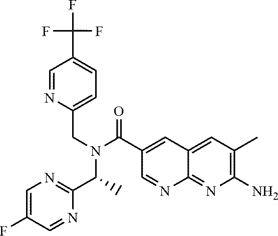 [SMILES: Cc1cc2cc(C(=O)N(Cc3ccc(C(F)(F)F)cn3)[C@H](C)c3ncc(F)cn3)cnc2nc1N] | 7-amino-N-((1R)-1-(5-fluoro-2-pyrimidinyl)ethyl)-6-methyl-N-((5-(trifluoromethyl)-2-pyridinyl)methyl)-1,8-naphthyridine-3-carboxamide | 2nd Peak. Regis Whelk-01 (R,R) columns (500 × 21 mm, 5 µm). Mobile phase: 70:30. B: 0.2% TEA in MeOH. | 485.9 |
| 482 | 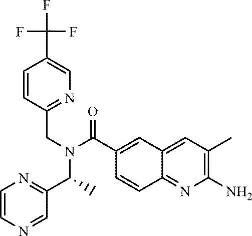 [SMILES: Cc1cc2cc(C(=O)N(Cc3ccc(C(F)(F)F)cn3)[C@H](C)c3cnccn3)ccc2nc1N] | 2-amino-3-methyl-N-((1R)-1-(2-pyrazinyl)ethyl)-N-((5-(trifluoromethyl)-2-pyridinyl)methyl)-6-quinolinecarboxamide | 1st Peak. Regis (R,R) Whelk-01 (250 × 21 mm, 5 µm). Mobile phase: 70:30. B: EtOH. | 467.1 |
| 483 | 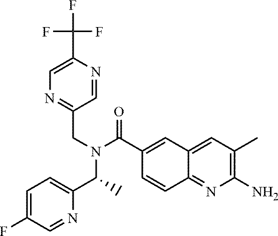 [SMILES: Cc1cc2cc(C(=O)N(Cc3cnc(C(F)(F)F)cn3)[C@H](C)c3ccc(F)cn3)ccc2nc1N] | 2-amino-N-((1R)-1-(5-fluoro-2-pyridinyl)ethyl)-3-methyl-N-((5-(trifluoromethyl)-2-pyrazinyl)methyl)-6-quinolinecarboxamide | 1st Peak. SeaPak OD (250 × 21 mm, 5 µm). Mobile phase: 80:20. B: MeOH. | 485.1 |
| 484 | 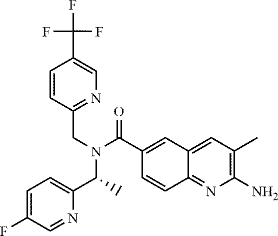 [SMILES: Cc1cc2cc(C(=O)N(Cc3ccc(C(F)(F)F)cn3)[C@H](C)c3ccc(F)cn3)ccc2nc1N] | 2-amino-N-((1R)-1-(5-fluoro-2-pyridinyl)ethyl)-3-methyl-N-((5-(trifluoromethyl)-2-pyridinyl)methyl)-6-quinolinecarboxamide | 1st Peak. Chiral Technology AD (250 × 21 mm, 5 µm). Mobile phase: 85:15. B: MeOH. | 484.2 |
| 485 | 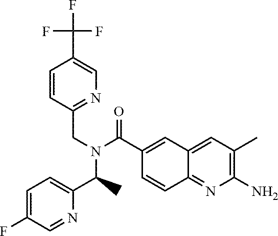 [SMILES: Cc1cc2cc(C(=O)N(Cc3ccc(C(F)(F)F)cn3)[C@@H](C)c3ccc(F)cn3)ccc2nc1N] | 2-amino-N-((1S)-1-(5-fluoro-2-pyridinyl)ethyl)-3-methyl-N-((5-(trifluoromethyl)-2-pyridinyl)methyl)-6-quinolinecarboxamide | 2nd Peak. Chiral Technology AD (250 × 21 mm, 5 µm). Mobile phase: 85:15. B: MeOH. | 484.2 |
| 486 | 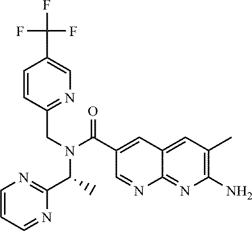 [SMILES: Cc1cc2cc(C(=O)N(Cc3ccc(C(F)(F)F)cn3)[C@H](C)c3ncccn3)cnc2nc1N] | 7-amino-6-methyl-N-((1R)-1-(2-pyrimidinyl)ethyl)-N-((5 -(trifluoromethyl)-2-pyridinyl)methyl)- 1,8-naphthyridine-3-carboxamide | 1st Peak. Chiral Technologies OX (250 × 30 mm, 5 µm). Mobile phase: 60:40. B: 0.2% TEA in MeOH. | 468.1 |
| 487 | 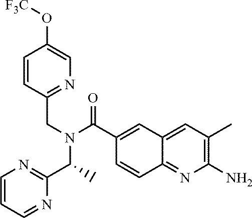 [SMILES: Cc1cc2cc(C(=O)N(Cc3ccc(OC(F)(F)F)cn3)[C@H](C)c3ncccn3)ccc2nc1N] | 2-amino-3-methyl-N-((1R)-1-(2-pyrimidinyl)ethyl)-N-((5 -(trifluoromethoxy)-2-pyridinyl)methyl)-6-quinolinecarboxamide | 2nd Peak. Chiral Technologies OX (250 × 30 mm, 5 µm). Mobile phase: 50:50. B: 0.2% TEA in MeOH. | 483.3 |
| 488 | 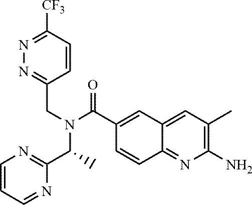 [SMILES: Cc1cc2cc(C(=O)N(Cc3ccc(C(F)(F)F)nn3)[C@H](C)c3ncccn3)ccc2nc1N] | 2-amino-3-methyl-N-((1R)-1-(2-pyrimidinyl)ethyl)-N-((6-(trifluoromethyl)-3 -pyridazinyl)methyl)-6-quinolinecarboxamide | 2nd Peak. Regis (R,R) Whelk-01 (250 × 30 mm, 5 µm). Mobile phase: 50:50. B: 0.2% TEA in MeOH. | 468.2 |
| 489 | 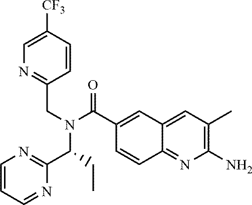 [SMILES: CC[C@H](c1ncccn1)N(Cc1ccc(C(F)(F)F)cn1)C(=O)c1ccc2nc(N)c(C)cc2c1] | 2-amino-3-methyl-N-((1R)-1-(2-pyrimidinyl)propyl)-N-((5 -(trifluoromethyl)-2-pyridinyl)methyl)-6-quinolinecarboxamide | 1st Peak. Chiral Technologies OX (250 × 30 mm, 5 mm). Mobile phase: 60:40. B: 0.2% TEA in MeOH. | 481.3 |
| 490 | 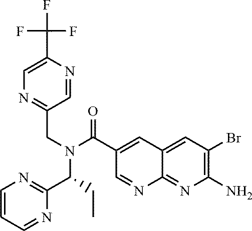 [SMILES: CC[C@H](c1ncccn1)N(Cc1cnc(C(F)(F)F)cn1)C(=O)c1cnc2nc(N)c(Br)cc2c1] | 7-amino-6-bromo-N-((1R)-1-(2-pyrimidinyl)ethyl)-N-((5 -(trifluoromethyl)-2-pyrazinyl)methyl)-1, 8-naphthyridine-3-carboxamide | 1st Peak. Regis (R,R) Whelk-01 (250 × 30 mm, 5 µm). Mobile phase: 60:40. B: 0.2% TEA in MeOH. | 533/535 |
| 491 | 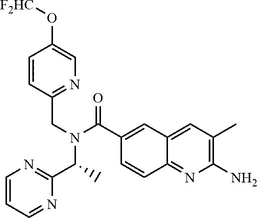 [SMILES: Cc1cc2cc(C(=O)N(Cc3ccc(OC(F)F)cn3)[C@H](C)c3ncccn3)ccc2nc1N] | 2-amino-N-((5-(difluoromethoxy)-2-pyridinyl)methyl)-3-methyl-N-((1R)-1-(2-pyrimidinyl)ethyl)-6-quinolinecarboxamide | 2nd Peak. Chiral Technologies OX (250 × 30 mm, 5 µm). Mobile phase: 50:50. B: 0.2% TEA in MeOH. | 465 |
| 492 | 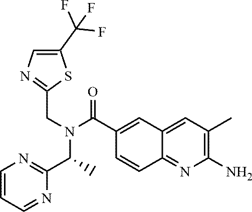 [SMILES: Cc1cc2cc(C(=O)N(Cc3ncc(C(F)(F)F)s3)[C@H](C)c3ncccn3)ccc2nc1N] | 2-amino-3-methyl-N-((1R)-1-(2-pyrimidinyl)ethyl)-N-((5 -(trifluoromethyl)-1,3-thiazol-2-yl)methyl)-6-quinolinecarboxamide | 2nd Peak. Regis (R,R) Whelk-01 (250 × 30 mm, 5 µm). Mobile phase: 65:35. B: 0.2% TEA in MeOH. | 473.1 |
| 493 | 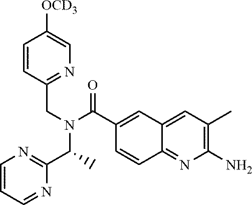 | 2-amino-3-methyl-N-((5-((~2~H_3_)methyloxy) -2-pyridinyl)methyl)-N-((1R)-1-(2-pyrimidinyl)ethyl)-6-quinolinecarboxamide | 1st Peak. Chiral Technologies AS (250 × 21 mm, 5 µm). Mobile phase: 85:15. B: 0.2% TEA in EtOH. | 432.2 |
| 494 | 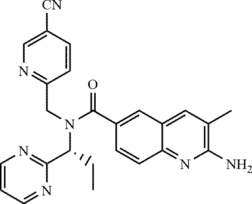 [SMILES: CC[C@H](c1ncccn1)N(Cc1ccc(C#N)cn1)C(=O)c1ccc2nc(N)c(C)cc2c1] | 2-amino-N-((5-cyano-2-pyridinyl)methyl)-3-methyl-N-((1R)-1-(2-pyrimidinyl)propyl)-6-quinolinecarboxamide | 1st Peak. Chiral Technologies AS (50 × 21 mm, 5 µm). Mobile phase: 80:20. B: 0.2% TEA in EtOH. | 438.2 |
| 495 | 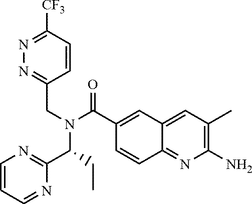 [SMILES: CC[C@H](c1ncccn1)N(Cc1ccc(C(F)(F)F)nn1)C(=O)c1ccc2nc(N)c(C)cc2c1] | 2-amino-3-methyl-N-((1R)-1-(2-pyrimidinyl)propyl)-N-((6-(trifluoromethyl)-3 -pyridazinyl)methyl)-6-quinolinecarboxamide | 1st Peak. Chiral Technologies OD (250 × 21 mm, 5 µm). Mobile phase: 75:25. B: 0.2% TEA in MeOH. | 482.1 |
| 496 | 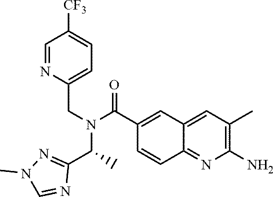 [SMILES: Cc1cc2cc(C(=O)N(Cc3ccc(C(F)(F)F)cn3)[C@H](C)c3ncn(C)n3)ccc2nc1N] | 2-amino-3-methyl-N-((1R)-1-(1-methyl-1H-1,2,4-triazol-3-yl)ethyl)-N-((5-(trifluoromethyl)-2-pyridinyl)methyl)-6-quinolinecarboxamide | 1st Peak. Chiral Technologies OD (250 × 21 mm, 5 mm). Mobile phase: 75:25. B: 0.2% TEA in MeOH. | 470.4 |
| 497 | 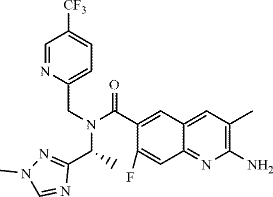 [SMILES: Cc1cc2cc(C(=O)N(Cc3ccc(C(F)(F)F)cn3)[C@H](C)c3ncn(C)n3)c(F)cc2nc1N] | 2-amino-7-fluoro-3-methyl-N-((1R)-1-(1-methyl-1H-1,2,4-triazol-3 -yl)ethyl)-N-((5-(trifluoromethyl)-2-pyridinyl)methyl)-6-quinolinecarboxamide | 2nd Peak. Chiral Technologies OD (250 × 21 mm, 5 µm). Mobile phase: 80:20. B: 0.2% TEA in EtOH. | 488.2 |
| 498 | 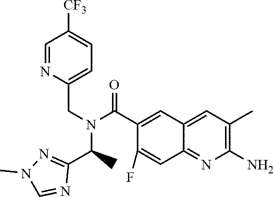 [SMILES: Cc1cc2cc(C(=O)N(Cc3ccc(C(F)(F)F)cn3)[C@@H](C)c3ncn(C)n3)c(F)cc2nc1N] | 2-amino-7-fluoro-3-methyl-N-((1S)-1-(1-methyl-1H-1,2,4-triazol-3 -yl)ethyl)-N-((5-(trifluoromethyl)-2-pyridinyl)methyl)-6-quinolinecarboxamide | 1st Peak. Chiral Technologies OD (250 × 21 mm, 5 µm). Mobile phase: 80:20. B: 0.2% TEA in EtOH. | 488.2 |
| 499 | 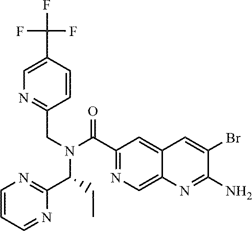 [SMILES: CC[C@H](c1ncccn1)N(Cc1ccc(C(F)(F)F)cn1)C(=O)c1cc2cc(Br)c(N)nc2cn1] | 2-amino-3-bromo-N-((1R)-1-(2-pyrimidinyl)propyl)-N-((5 -(trifluoromethyl)-2-pyridinyl)methyl)- 1,7-naphthyridine-6-carboxamide | 1st peak. Chiralcel OX-H (21 × 250 mm, 5 µm); Mobile Phase: 75:35; B: 0.2% TEA in MeOH | 546/548 |
| 500 | 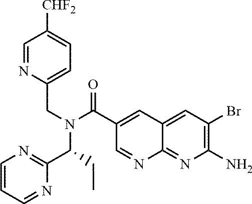 [SMILES: CC[C@H](c1ncccn1)N(Cc1ccc(C(F)F)cn1)C(=O)c1cnc2nc(N)c(Br)cc2c1] | 7-amino-6-bromo-N-((5 -(difluoromethyl)-2-pyridinyl)methyl)-N-((1R)-1-(2-pyrimidinyl)propyl)-1,8-naphthyridine-3-carboxamide | 1st peak. Chiral Technologies OD (21 × 250 mm, 5 µm); Mobile Phase: 75:25; B: 0.2% TEA in MeOH. | 528/530 |
| 501 | 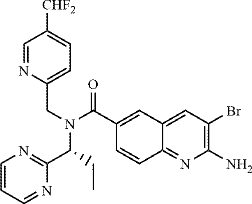 [SMILES: CC[C@H](c1ncccn1)N(Cc1ccc(C(F)F)cn1)C(=O)c1ccc2nc(N)c(Br)cc2c1] | 2-amino-3-bromo-N-((5 -(difluoromethyl)-2-pyridinyl)methyl)-N-((1R)-1-(2-pyrimidinyl)propyl)-6-quinolinecarboxamide | 1st peak. Chiral Technologies OX (21 × 250 mm, 5 µm); Mobile Phase: 50:50; B: 0.2% TEA in EtOH. | 527/529 |
| 774 | 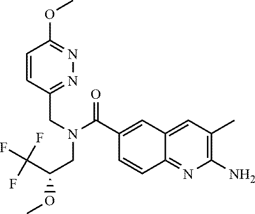 [SMILES: COc1ccc(CN(C[C@H](OC)C(F)(F)F)C(=O)c2ccc3nc(N)c(C)cc3c2)nn1] | 2-amino-N-((6-methoxy-3-pyridazinyl)methyl)-3 -methyl-N-((2S)-3,3,3-trifluoro-2-methoxypropyl)-6-quinolinecarboxamide | 1st peak. Chiral Technologies ID column (250 × 21 mm, 5 µm); mobile phase 75:25 iPrOH with 0.2% TEA. | 450.2 |
| 775 | 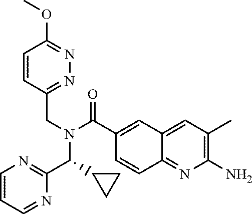 [SMILES: COc1ccc(CN(C(=O)c2ccc3nc(N)c(C)cc3c2)[C@@H](c2ncccn2)C2CC2)nn1] | 2-amino-N-((R)-cyclopropyl(2-pyrimidinyl)methyl)-N-((6-methoxy-3-pyridazinyl)methyl)-3 -methyl-6-quinolinecarboxamide | 1st peak. Chiral Technologies OJ column (250 × 21 mm, 5 µm); mobile phase 90:10 MeOH with 1.0% TEA | 456.2 |
| 776 | 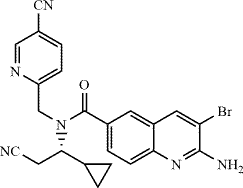 [SMILES: N#CC[C@@H](C1CC1)N(Cc1ccc(C#N)cn1)C(=O)c1ccc2nc(N)c(Br)cc2c1] | (S)-2-amino-3-bromo-N-(2-cyano-1-cyclopropylethyl)-N-((5-cyanopyridin-2-yl)methyl)quinoline-6-carboxamide | 2^{nd} peak. Chiral Technologies AS (250 × 21 mm, 5 µm); Mobile Phase: 75:25; B: 0.2% TEA in MeOH. | 475.2 |
| 777 | 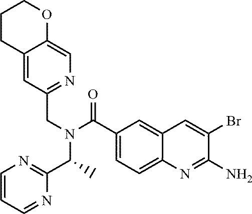 [SMILES: C[C@H](c1ncccn1)N(Cc1cc2c(cn1)OCCC2)C(=O)c1ccc2nc(N)c(Br)cc2c1] | (R)-2-amino-3-bromo-N-((3,4-dihydro-2H-pyrano[2,3-c]pyridin-6-yl)methyl)-N-(1-(pyrimidin-2-yl)ethyl)quinoline-6-carboxamide | 1^{st} peak. Chiral Technologies AS-H (250 × 21 mm, 5 µm); Mobile Phase: 60:40; B: 0.2% TEA in MeOH. | 519.2 |
| 778 | 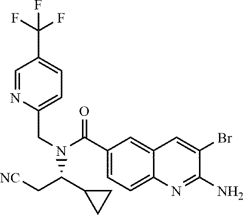 [SMILES: N#CC[C@@H](C1CC1)N(Cc1ccc(C(F)(F)F)cn1)C(=O)c1ccc2nc(N)c(Br)cc2c1] | (S)-2-amino-3-bromo-N-(2-cyano-1-cyclopropylethyl)-N-((5-(trifluoromethyl)pyridi n-2-yl)methyl)quinoline-6-carboxamide | 1^{st} peak. Chiral Technologies AS (250 × 21 mm, 5 µm); Mobile Phase: 90:10; B: 0.2% TEA in MeOH. | 518.0 |
| 780 | 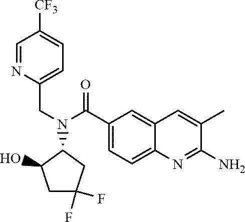 [SMILES: Cc1cc2cc(C(=O)N(Cc3ccc(C(F)(F)F)cn3)[C@@H]3CC(F)(F)C[C@H]3O)ccc2nc1N] | 2-amino-N-((1R,2R)-4,4-difluoro-2-hydroxycyclopentyl)-3-methyl-N-((5-(trifluoromethyl)-2-pyridinyl)methyl)-6-quinolinecarboxamide | 2nd peak. Chiral Technologies IC column (250 × 21 mm, 5 mm) with a mobile phase of 65% Liquid CO2 and 35% MeOH with 0.2% TEA using a flowrate of 80 mL/min. | 481.2 |
| 781 | 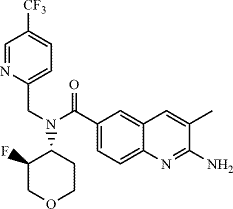 [SMILES: Cc1cc2cc(C(=O)N(Cc3ccc(C(F)(F)F)cn3)[C@@H]3CCOC[C@H]3F)ccc2nc1N] | 2-amino-N-((3S,4R)-3-fluorotetrahydro-2H-pyran-4-yl)-3 -methyl-N-((5-(trifluoromethyl)-2-pyridinyl)methyl)-6-quinolinecarboxamide | 2nd peak. Chiral Technologies OJ column (250 X 21 mm, 5 mm) with a mobile phase of 85% Liquid CO2 and 15% MeOH with 0.2% TEA using a flowrate of 100 mL/min. | 463.2 |
| 782 | 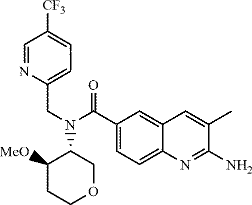 [SMILES: CO[C@@H]1CCOC[C@H]1N(Cc1ccc(C(F)(F)F)cn1)C(=O)c1ccc2nc(N)c(C)cc2c1] | 2-amino-N-((3R,4R)-4-methoxytetrahydro-2H-pyran-3-yl)-3-methyl-N-((5-(trifluoromethyl)-2-pyridinyl)methyl)-6-quinolinecarboxamide | 1st peak. Chiral Technologies AS column (250 X 21 mm, 5 mm) with a mobile phase of 85% Liquid CO2 and 15% MeOH with 0.2% TEA using a flowrate of 80 mL/min. | 475.2 |
| 784 | 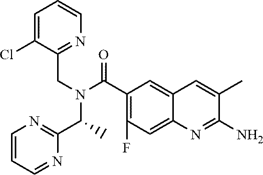 [SMILES: Cc1cc2cc(C(=O)N(Cc3ncccc3Cl)[C@H](C)c3ncccn3)c(F)cc2nc1N] | 2-amino-N-((3-chloro-2-pyridinyl)methyl)-7-fluoro-3-methyl-N-((1R)-1-(2-pyrimidinyl)ethyl)-6-quinolinecarboxamide | 1st peak. Chiral Technologies AS (1 00 x 2.1 mm, 3 µm) ; Mobile Phase: 75:25; B: 0.2% TEA in MeOH. | 451.2 |
| 785 | 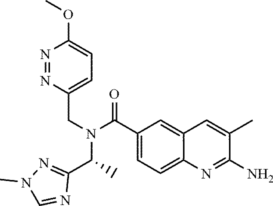 [SMILES: COc1ccc(CN(C(=O)c2ccc3nc(N)c(C)cc3c2)[C@H](C)c2ncn(C)n2)nn1] | (R)-2-amino-N-((6-methoxypyridazin-3-yl)methyl)-3-methyl-N-(1-(1-methyl-1H-1,2,4-triazol-3-yl)ethyl)quinoline-6-carboxamide | 1st peak. preparative SFC using a Chiral Technologies OD column (250 X 21 mm, 5 mm) with a mobile phase of 65% Liquid CO2 and 35% MeOH with 0.2% TEA using a flowrate of 80 mL/min. | 433 |
| 786 | 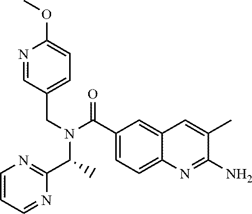 [SMILES: COc1ccc(CN(C(=O)c2ccc3nc(N)c(C)cc3c2)[C@H](C)c2ncccn2)cn1] | (R)-2-amino-N-((6-methoxypyridin-3-yl)methyl)-3-methyl-N-(1-(pyrimidin-2-yl)ethyl)quinoline-6-carboxamide | 1st peak. Chiral Technologies AS column (250 × 21 mm, 5 mm) with a mobile phase of 80% Liquid CO2 and 20% MeOH with 0.2% TEA using a flowrate of 80 mL/min. | 429 |
| 787 | 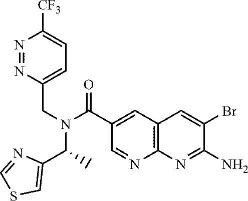 [SMILES: C[C@H](c1cscn1)N(Cc1ccc(C(F)(F)F)nn1)C(=O)c1cnc2nc(N)c(Br)cc2c1] | (R)-7-amino-6-bromo-N-(1-(thiazol-4-yl)ethyl)-N-((6-(trifluoromethyl)pyrida zin-3-yl)memthyl)-1,8-naphthyridine-3-carboxamide | Peak 1. Regis Whelk-01 (R,R) column (150 × 21 mm, 5 µm) with a mobile phase of 60% Liquid CO2 and 40% MeOH with 0.2% TEA using a flowrate of 80 mL/min. | 538/540 |
| 788 | 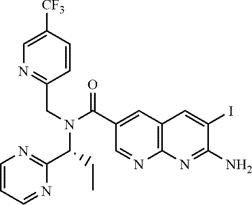 [SMILES: CC[C@H](c1ncccn1)N(Cc1ccc(C(F)(F)F)cn1)C(=O)c1cnc2nc(N)c(I)cc2c1] | (R)-7-amino-6-iodo-N-(1-(pyrimidin-2-yl)propyl)-N-((5-(trifluoromethyl)pyridi n-2-yl)methyl)-1,8-naphthyridine-3-carboxamide | Peak 1. Chiral Technologies OD column (250 × 21 mm, 5 µm) with a mobile phase of 80% Liquid CO2 and 20% MeOH with 0.2% TEA using a flowrate of 80 mL/min. | 594 |

Example 502. (R)-2-amino-N-((5-cyanopyridin-2-yl)methyl)-3-methyl-N-(1-(pyrimidin-2-yl)ethyl)quinoline-6-carboxamide. (Method B)

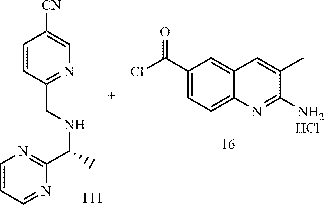

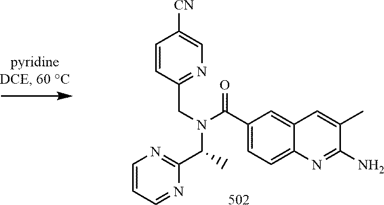

To a 150-mL round-bottomed flask was added (R)-6-(((1-(pyrimidin-2-yl)ethyl)amino)methyl)nicotinonitrile (111, 1.00 g, 4.18 mmol) and 2-amino-3-methylquinoline-6-carbonyl chloride hydrochloride (16, 1.34 g, 5.22 mmol) in 1,2-dichloroethane (41.8 mL). Then pyridine (3.55 mL, 41.8 mmol) was added to the reaction mixture. The reaction mixture was heated at 60° C., then cooled to RT and treated with saturated NaHCO₃ and a small amount of MeOH. The mixture was diluted with DCM. The layers were separated, and the aqueous layer was extracted with DCM (3x). The combined organic extracts were dried over MgSO₄, filtered and concentrated in vacuo. The crude material was purified by silica gel chromatography (0% to 100% 3:1 EtOAc:EtOH in heptane) to provide the title compound (502, 1.35 g, 3.19 mmol, 76% yield) as a light-yellow solid. m/z (ESI): 424.1 (M+H)⁺. ¹H NMR (400 MHz, DMSO-d₆) δ ppm 8.89 (d, J=1.5 Hz, 1 H), 8.77 (d, J=4.8 Hz, 2 H), 8.20 (br d, J=7.7 Hz, 1 H), 7.82 (s, 1 H), 7.79 (s, 1 H), 7.59 (br s, 1 H), 7.53 (br d, J=8.4 Hz, 2 H), 7.39 (t, J=4.9 Hz, 1 H), 6.48 (br s, 2 H), 5.43 (br s, 1 H), 4.89 (br d, J=17.3 Hz, 1 H), 4.51 (br d, J=15.7 Hz, 1 H), 2.21 (br s, 3 H), 1.61 (br d, J=6.7 Hz, 3 H).

TABLE 11

Examples below were prepared in a manner similar to that described above (General Method B) for Example 502

| Ex. # | Chemical Structure | Name | LRMS: (ESI + ve ion) m/z |
|---|---|---|---|
| 503 | 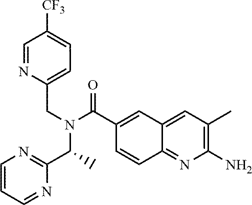 [SMILES: Cc1cc2cc(C(=O)N(Cc3ccc(C(F)(F)F)cn3)[C@H](C)c3ncccn3)ccc2nc1N] | 2-amino-3-methyl-N-((1R)-1-(2-pyrimidinyl)ethyl)-N-((5-(trifluoromethyl)-2-pyridinyl)methyl)-6-quinolinecarboxamide | 467.2 |
| 504 | 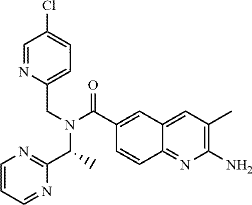 [SMILES: Cc1cc2cc(C(=O)N(Cc3ccc(Cl)cn3)[C@H](C)c3ncccn3)ccc2nc1N] | 2-amino-N-((5-chloro-2-pyridinyl)methyl)-3-methyl-N-((1R)-1-(2-pyrimidinyl)ethyl)-6-quinolinecarboxamide | 433 |
| 505 | 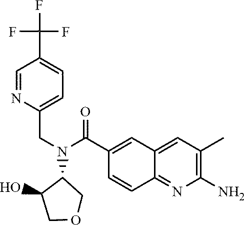 [SMILES: Cc1cc2cc(C(=O)N(Cc3ccc(C(F)(F)F)cn3)[C@@H]3COC[C@H]3O)ccc2nc1N] | 2-amino-N-((3R,4S)-4-hydroxytetrahydro-3-furanyl)-3-methyl-N-((5-(trifluoromethyl)-2-pyridinyl)methyl)-6-quinolinecarboxamide | 447.2 |
| 506 | 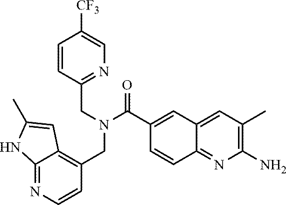 | 2-amino-3-methyl-N-((2-methyl-1H-pyrrolo[2,3-b]pyridin-4-yl)methyl)-N-((5-(trifluoromethyl)-2-pyridinyl)methyl)-6-quinolinecarboxamide | 505 |
| 507 | 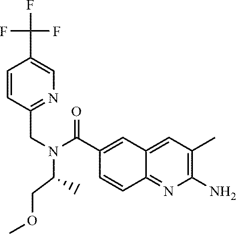 [SMILES: COC[C@@H](C)N(Cc1ccc(C(F)(F)F)cn1)C(=O)c1ccc2nc(N)c(C)cc2c1] | 2-amino-N-((2R)-1-methoxy-2-propanyl)-3-methyl-N-((5-(trifluoromethyl)-2-pyridinyl)methyl)-6-quinolinecarboxamide | 433 |
| 508 | 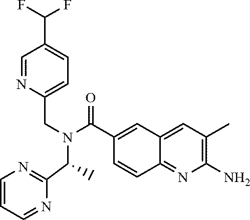 [SMILES: Cc1cc2cc(C(=O)N(Cc3ccc(C(F)F)cn3)[C@H](C)c3ncccn3)ccc2nc1N] | 2-amino-N-((5-(difluoromethyl)-2-pyridinyl)methyl)-3-methyl-N-((1R)-1-(2-pyrimidinyl)ethyl)-6-quinolinecarboxamide | 449.1 |
| 509 | 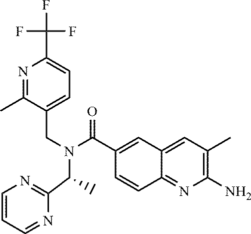 | 2-amino-3-methyl-N-((2-methyl-6-(trifluoromethyl)-3-pyridinyl)methyl)-N-((1R)-1-(2-pyrimidinyl)ethyl)-6-quinolinecarboxamide | 481.2 |
| 510 | 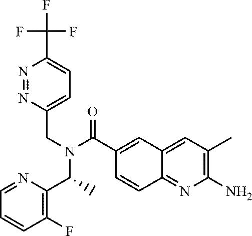 [SMILES: Cc1cc2cc(C(=O)N(Cc3ccc(C(F)(F)F)nn3)[C@H](C)c3ncccc3F)ccc2nc1N] | 2-amino-N-((1R)-1-(3-fluoro-2-pyridinyl)ethyl)-3-methyl-N-((6-(trifluoromethyl)-3-pyridazinyl)methyl)-6-quinolinecarboxamide | 485.2 |
| 511 | 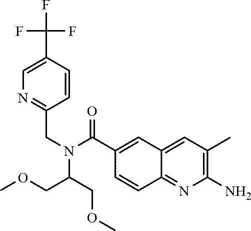 [SMILES: COCC(COC)N(Cc1ccc(C(F)(F)F)cn1)C(=O)c1ccc2nc(N)c(C)cc2c1] | 2-amino-N-(1,3-dimethoxy-2-propanyl)-3-methyl-N-((5-(trifluoromethyl)-2-pyridinyl)methyl)-6-quinolinecarboxamide | 463.2 |
| 512 | 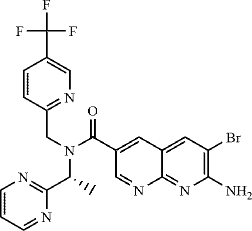 [SMILES: C[C@H](c1ncccn1)N(Cc1ccc(C(F)(F)F)cn1)C(=O)c1cnc2nc(N)c(Br)cc2c1] | 7-amino-6-bromo-N-((1R)-1-(2-pyrimidinyl)ethyl)-N-((5-(trifluoromethyl)-2-pyridinyl)methyl)-1,8-naphthyridine-3-carboxamide | 532/534 |
| 513 | 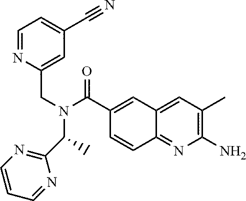 [SMILES: Cc1cc2cc(C(=O)N(Cc3cc(C#N)ccn3)[C@H](C)c3ncccn3)ccc2nc1N] | 2-amino-N-((4-cyano-2-pyridinyl)methyl)-3-methyl-N-((1R)-1-(2-pyrimidinyl)ethyl)-6-quinolinecarboxamide | 424 |
| 514 | 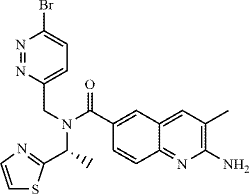 [SMILES: Cc1cc2cc(C(=O)N(Cc3ccc(Br)nn3)[C@H](C)c3nccs3)ccc2nc1N] | 2-amino-N-((6-bromo-3-pyridazinyl)methyl)-3-methyl-N-((1R)-1-(1,3-thiazol-2-yl)ethyl)-6-quinolinecarboxamide | 483/485 |
| 789 | 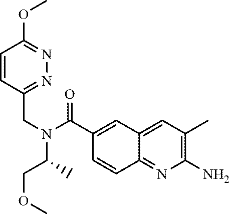 [SMILES: COC[C@@H](C)N(Cc1ccc(OC)nn1)C(=O)c1ccc2nc(N)c(C)cc2c1] | 2-amino-N-((2R)-1 -methoxy-2-propanyl)-N-((6-methoxy-3-pyridazinyl)methyl)-3-methyl-6-quinolinecarboxamide | 396.0 |
| 790 | 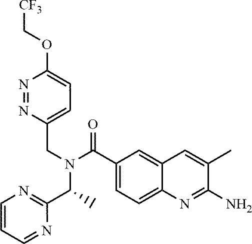 [SMILES: Cc1cc2cc(C(=O)N(Cc3ccc(OCC(F)(F)F)nn3)[C@H](C)c3ncccn3)ccc2nc1N] | (R)-2-amino-3-methyl-N-(1-(pyrimidin-2-yl)ethyl)-N-((6-(2,2,2-trifluoroethoxy)pyridazin-3-yl)methyl) quinoline-6-carboxamide | 498.2 |
| 791 | 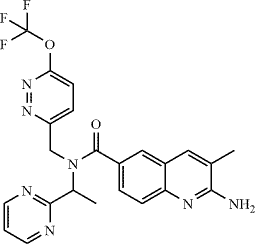 [SMILES: Cc1cc2cc(C(=O)N(Cc3ccc(OC(F)(F)F)nn3)C(C)c3ncccn3)ccc2nc1N] | 2-amino-3-methyl-N-(1-(pyrimidin-2-yl)ethyl)-N-((6-(trifluoromethoxy)pyridazin-3-yl)methyl)quinoline-6-carboxamide | 484.2 |
| 792 | 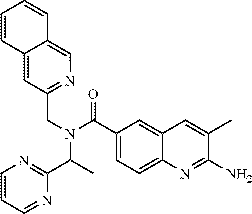 [SMILES: Cc1cc2cc(C(=O)N(Cc3cc4ccccc4cn3)C(C)c3ncccn3)ccc2nc1N] | 2-amino-N-(isoquinolin-3-ylmethyl)-3-methyl-N-(1-(pyrimidin-2-yl)ethyl)quinoline-6-carboxamide | 449.0 |
| 793 | | (R)-7-amino-6-bromo-N-(1 -(pyrimidin-2-yl)ethyl)-N-((6-(2,2,2-trifluoroethoxy)pyridazin-3-yl)methyl)-1,8-naphthyridine-3-carboxamide | 563.0 |
| 795 | 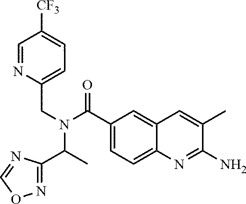 [SMILES: Cc1cc2cc(C(=O)N(Cc3ccc(C(F)(F)F)cn3)C(C)c3ncon3)ccc2nc1N] | N-(1-(1,2,4-oxadiazol-3-yl)ethyl)-2-amino-3-methyl-N-((5-(trifluoromethyl)pyridin-2-yl)methyl)quinoline-6-carboxamide | 457.1 |
| 796 | 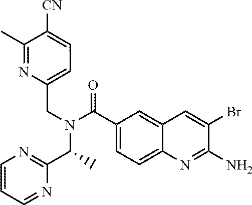 [SMILES: Cc1nc(CN(C(=O)c2ccc3nc(N)c(Br)cc3c2)[C@H](C)c2ncccn2)ccc1C#N] | (R)-2-amino-3-bromo-N-((5-cyano-6-methylpyridin-2-yl)methyl)-N-(1-(pyrimidin-2-yl)ethyl)quinoline-6-carboxamide | 502/504 |
| 797 | 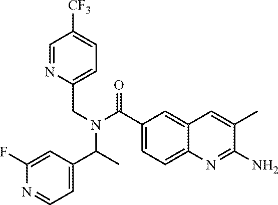 [SMILES: Cc1cc2cc(C(=O)N(Cc3ccc(C(F)(F)F)cn3)C(C)c3ccnc(F)c3)ccc2nc1N] | 2-amino-N-(1-(2-fluoropyridin-4-yl)ethyl)-3-methyl-N-((5-(trifluoromethyl)pyridin-2-yl)methyl)quinoline-6-carboxamide | 484.0 |
| 798 | 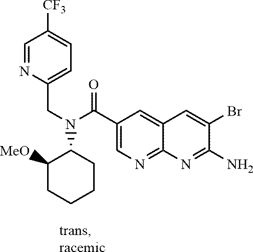 | 7-amino-6-bromo-N-((1R,2R)-2-methoxycyclohexyl)-N-((5-(trifluoromethyl)pyridin-2-yl)methyl)-1,8-naphthyridine-3-carboxamide | 538.2/540.2 |
| 799 | 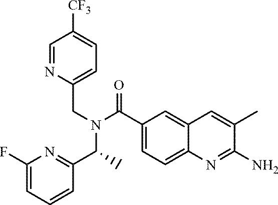 [SMILES: Cc1cc2cc(C(=O)N(Cc3ccc(C(F)(F)F)cn3)[C@H](C)c3cccc(F)n3)ccc2nc1N] | (R)-2-amino-N-(1 -(6-fluoropyridin-2-yl)ethyl)-3-methyl-N-((5-(trifluoromethyl)pyridin-2-yl)methyl)quinoline-6-carboxamide | 484.2 |
| 800 | 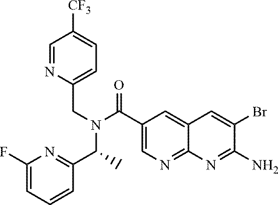 [SMILES: C[C@H](c1cccc(F)n1)N(Cc1ccc(C(F)(F)F)cn1)C(=O)c1cnc2nc(N)c(Br)cc2c1] | (R)-7-amino-6-bromo-N-(1 -(6-fluoropyridin-2-yl)ethyl)-N-((5-(trifluoromethyl)pyridin-2-yl)methyl)-1,8-naphthyridine-3-carboxamide | 549.2/551.2 |
| 801 | | 2-amino-N-((5-(2-hydroxy-2-propanyl)-2-pyridinyl)methyl)-3-methyl-N-((1R)-1-(2-pyrimidinyl)ethyl)-6-quinolinecarboxamide | 457.3 |
| 802 | | 2-amino-3-methyl-N-(4-(3-oxetanyl)benzyl)-N-((1R)-1-(2-pyrimidinyl)ethyl)-6-quinolinecarboxamide, 2-amino-3-methyl-N-(4-(3-oxetanyl)benzyl)-N-((1S)-1-(2-pyrimidinyl)ethyl)-6-quinolinecarboxamide | 454.3 |
| 803 | 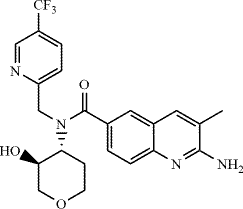 [SMILES: Cc1cc2cc(C(=O)N(Cc3ccc(C(F)(F)F)cn3)[C@@H]3CCOC[C@H]3O)ccc2nc1N] | 2-amino-N-((3R,4S)-3-hydroxytetrahydro-2H-pyran-4-yl)-3-methyl-N-((5-(trifluoromethyl)-2-pyridinyl)methyl)-6-quinolinecarboxamide and 2-amino-N-((3S,4R)-3-hydroxytetrahydro-2H-pyran-4-yl)-3-methyl-N-((5-(trifluoromethyl)-2-pyridinyl)methyl)-6-quinolinecarboxamide | 461.2 |
| 804 | | 2-amino-7-chloro-N-((3-fluoro-2-pyridinyl)methyl)-3-methyl-N-((5-(trifluoromethyl)-2-pyridinyl)methyl)-6-quinolinecarboxamide | 503.9 |
| 805 | | 2-amino-3-methyl-N-(2-methyl-1-(pyrimidin-2-yl)propyl)-N-((5-(trifluoromethyl)pyridin-2-yl)methyl)quinoline-6-carboxamide | 495.2 |
| 806 | 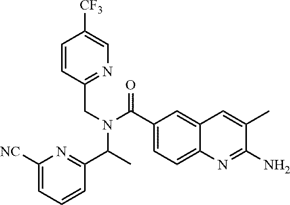 | 2-amino-N-(1-(6-cyanopyridin-2-yl)ethyl)-3-methyl-N-((5-(trifluoromethyl)pyridin-2-yl)methyl) quinoline-6-carboxamide | 491.2 |
| 807 | 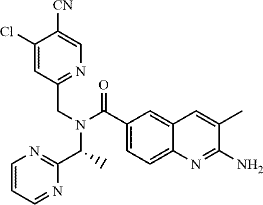 [SMILES: Cc1cc2cc(C(=O)N(Cc3cc(Cl)c(C#N)cn3)[C@H](C)c3ncccn3)ccc2nc1N] | (R)-2-amino-N-((4-chloro-5-cyanopyridin-2-yl)methyl)-3-methyl-N-(1-(pyrimidin-2-yl)ethyl)quinoline-6-carboxamide | 458.1 |
| 808 | 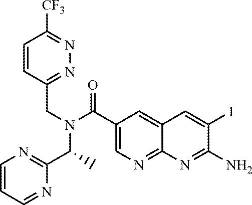 [SMILES: C[C@H](c1ncccn1)N(Cc1ccc(C(F)(F)F)nn1)C(=O)c1cnc2nc(N)c(I)cc2c1] | (R)-7-amino-6-iodo-N-(1-(pyrimidin-2-yl)ethyl)-N-((6-(trifluoromethyl)pyridazin-3-yl)methyl)-1,8-naphthyridine-3-carboxamide | 581.0 |

Example 515: (R)-2-amino-N-(6-fluoro-3,4-dihydro-2H-pyrano[3,2-b]pyridin-4-yl)-3-methyl-N-((6-(trifluoromethyl)pyridazin-3-yl)methyl)quinoline-6-carboxamide. (Method B-SFC)

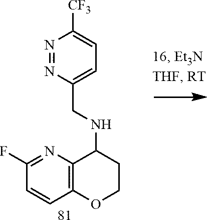

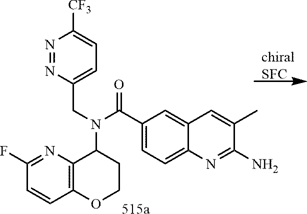

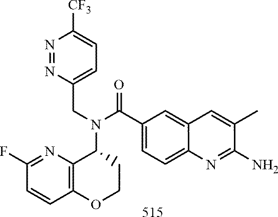

A mixture of triethylamine (0.52 mL, 3.77 mmol) and 6-fluoro-N-((6-(trifluoromethyl)pyridazin-3-yl)methyl)-3,4-dihydro-2H-pyrano[3,2-b]pyridin-4-amine (81, 206 mg, 0.62 mmol) in THF (2.5 mL) at RT was treated with 2-amino-3-methylquinoline-6-carbonyl chloride hydrochloride (16, 161 mg, 0.62 mmol) as a solid. The mixture was stirred for 30 min then concentrated in vacuo. The residue containing racemic 515a was subjected to chiral SFC under the conditions indicated below to give 2 eluents. Chiral SFC: Chiral Technologies OJ column (250 x 21 mm, 5 µm) with a mobile phase of 75% liquid CO₂ and 25% MeOH with 0.2% TEA using a flowrate of 80 mL/min. The more potent (measured by IC₅₀ in HCT116 MTAP null cell viability assay) enantiomer was assigned as the (R)—; the less potent (measured by IC₅₀ in HCT116 MTAP null cell viability assay) enantiomer was assigned as (S)—. The 1^{st} eluting peak was assigned as (R)-2-amino-N-(6-fluoro-3,4-dihydro-2H-pyrano [3,2-b]pyridin-4-yl)-3-methyl-N-((6-(trifluoromethyl)pyridazin-3-yl)methyl)quinoline-6-carboxamide (515, 53 mg, 0.10 mmol, 16% yield) as an off-white solid. m/z (ESI)— 513.0 (M+H)⁺. ¹H NMR (400 MHz, DMSO-d6) δ ppm 8.23 (br d, J=8.57 Hz, 1 H) 8.02 - 8.19 (m, 1 H) 7.85 - 8.00 (m, 1 H) 7.73 - 7.85 (m, 1 H) 7.66 (br d, J=7.73 Hz, 1 H) 7.35 - 7.58 (m, 2 H) 7.00 (br dd, J=8.57, 2.72 Hz, 1 H) 6.52 (br s, 2 H) 5.38 - 5.45 (m, 1 H) 4.93 (br d, J=16.10 Hz, 1 H) 4.48 (br d, J=16.51 Hz, 1 H) 4.32 (br d, J= 11.29 Hz, 1 H) 4.10 - 4.23 (m, 1 H) 2.32 - 2.47 (m, 2 H) 2.22 (s, 3 H). The 2^{nd} eluting peak was assigned as (S)-2-amino-N-(6-fluoro-3,4-dihydro-2H-pyrano[3,2-b]pyridin-4-yl)-3-methyl-N-((6-(trifluoromethyl)pyridazin-3-yl)methyl)quinoline-6-carboxamide (55 mg, 0.107 mmol, 17% yield). m/z (ESI): 513.1(M+H)⁺. 1H NMR (400 MHz, DMSO-d6) δ ppm 8.23 (br d, J=8.57 Hz, 1 H) 8.12 (br d, J=8.78 Hz, 1 H) 7.88 (br s, 1 H) 7.81 (br s, 1 H) 7.66 (br d, J=7.73 Hz, 1 H) 7.51 (br d, J=8.36 Hz, 1 H) 7.43 (dd, J=8.57, 6.69 Hz, 1 H) 7.00 (br dd, J=8.47, 2.61 Hz, 1 H) 6.50 (br s, 2 H) 5.37 - 5.45 (m, 1 H) 4.93 (br d, J=16.30 Hz, 1 H) 4.48 (br d, J=16.72 Hz, 1 H) 4.25 - 4.39 (m, 1 H) 4.10 - 4.23 (m, 1 H) 2.32 -2.47 (m, 2 H) 2.22 (s, 3 H).

TABLE 12

Examples below were prepared in a manner similar to that described above (Method B-SFC) for Example 515

| | | | | |
|---|---|---|---|---|
| Ex. # | Chemical Structure | Name | Peak sequence and SFC conditions | LRMS: (ESI + ve ion) m/z |
| 516 | 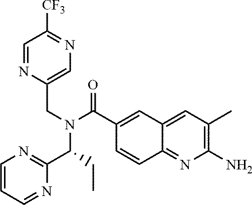 [SMILES: CC[C@H](c1ncccn1)N(Cc1cnc(C(F)(F)F)cn1)C(=O)c1ccc2nc(N)c(C)cc2c1] | 2-amino-3-methyl-N-((1R)-1-(2-pyrimidinyl)propyl)-N-((5-(trifluoromethyl)-2-pyrazinyl)methyl)-6-quinolinecarboxamide | Peak 1. Chiral Technologies AS (250 × 21 mm, 5 um); Mobile phase: 85:15; B: 0.2% TEA in EtOH. | 482.1 |
| 517 | 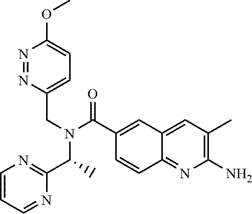 [SMILES: COc1ccc(CN(C(=O)c2ccc3nc(N)c(C)cc3c2)[C@H](C)c2ncccn2)nn1] | 2-amino-N-((6-methoxy-3-pyridazinyl)methyl)-3-methyl-N-((IR)-1-(2-pyrimidinyl)ethyl)-6-quinolinecarboxamide | Peak 1. Chiral Technologies AD column (250 × 21 mm, 5 mm). Mobile phase: 70:30. B: 0.2% TEA in EtOH. | 430 |
| 518 | 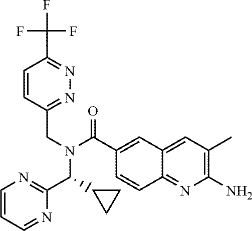 [SMILES: Cc1cc2cc(C(=O)N(Cc3ccc(C(F)(F)F)nn3)[C@@H](c3ncccn3)C3CC3)ccc2nc1N] | 2-amino-N-((R)-cyclopropyl(2-pyrimidinyl)methyl)-3 -methyl-N-((6-(trifluoromethyl)-3-pyridazinyl)methyl)-6-quinolinecarboxamide | 1st Peak: Chiral Technologies OJ column (250 × 21 mm, 5 mm). Mobile phase: 83:17. B: 0.2% TEA in MeOH. | 494.1 |
| 519 | 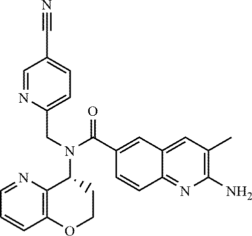 [SMILES: Cc1cc2cc(C(=O)N(Cc3ccc(C#N)cn3)[C@@H]3CCOc4cccnc43)ccc2nc1N] | 2-amino-N-((5-cyano-2-pyridinyl)methyl)-N-((4R)-3,4-dihydro-2H-pyrano[3,2-b]pyridin-4-yl)-3-methyl-6-quinolinecarboxamide | 1st Peak. Chiral Technologies IA (250 × 21 mm, 5 mm). Mobile phase: 65:35. B: 0.2% TEA in EtOH. | 451 |
| 520 | 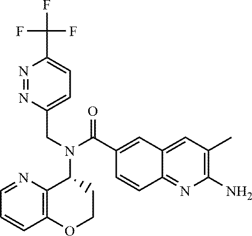 [SMILES: Cc1cc2cc(C(=O)N(Cc3ccc(C(F)(F)F)nn3)[C@@H]3CCOc4cccnc43)ccc2nc1N] | 2-amino-N-((4R)-3,4-dihydro-2H-pyrano[3,2-b]pyridin-4-yl)-3-methyl-N-((6-(trifluoromethyl)-3 -pyridazinyl)methyl)-6-quinolinecarboxamide | 1st peak, Chiral Technologies AD column (250 × 21 mm, 5 mm) with a mobile phase of 65% liquid CO₂ and 35% EtOH with 0.2% TEA | 495.2 |
| 521 | 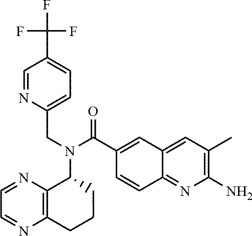 [SMILES: Cc1cc2cc(C(=O)N(Cc3ccc(C(F)(F)F)cn3)[C@@H]3CCCc4nccnc43)ccc2nc1N] | 2-amino-3-methyl-N-((5R)-5,6,7,8-tetrahydro-5-quinoxalinyl)-N-((5-(trifluoromethyl)-2-pyridinyl)methyl)-6-quinolinecarboxamide | 1st peak, Chiral Technologies IA column (250 × 30 mm, 5 mm) with a mobile phase of 70% liquid CO₂ and 30% EtOH with 0.2% TEA | 493.1 |
| 522 | 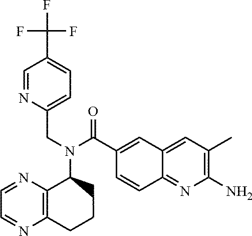 [SMILES: Cc1cc2cc(C(=O)N(Cc3ccc(C(F)(F)F)cn3)[C@H]3CCCc4nccnc43)ccc2nc1N] | 2-amino-3-methyl-N-((5 S)-5,6,7,8-tetrahydro-5-quinoxalinyl)-N-((5-(trifluoromethyl)-2-pyridinyl)methyl)-6-quinolinecarboxamide | 2nd peak, Chiral Technologies IA column (250 × 30 mm, 5 mm) with a mobile phase of 70% liquid CO₂ and 30% EtOH with 0.2% TEA | 493.1 |
| 523 | 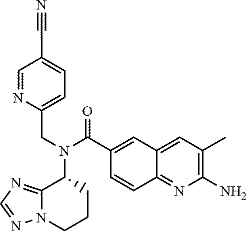 [SMILES: Cc1cc2cc(C(=O)N(Cc3ccc(C#N)cn3)[C@@H]3CCCn4ncnc43)ccc2nc1N] | 2-amino-N-((5-cyano-2-pyridinyl)methyl)-3-methyl-N-((8R)-5,6,7,8-tetrahydro [1,2,4]triazolo [1,5-a]pyridin-8-yl)-6-quinolinecarboxamide | 1st peak, Chiral Technologies AS column (250 × 21 mm, 5 mm) with a mobile phase of 70% liquid CO₂ and 30% MeOH with 0.2% TEA | 439.2 |
| 524 | 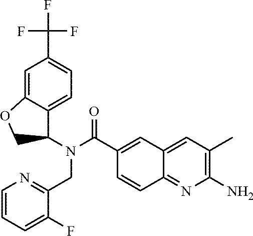 [SMILES: Cc1cc2cc(C(=O)N(Cc3ncccc3F)[C@H]3COc4cc(C(F)(F)F)ccc43)ccc2nc1N] | 2-amino-N-((3-fluoro-2-pyridinyl)methyl)-3-methyl-N-((3R)-6-(trifluoromethyl)-2,3-dihydro-1-benzofuran-3-yl)-6-quinolinecarboxamide | 1st peak, Chiral Technologies AS column (250 × 21 mm, 5 mm) with a mobile phase of 75% liquid CO₂ and 25% MeOH with 0.2% TEA | 497.2 |
| 525 | 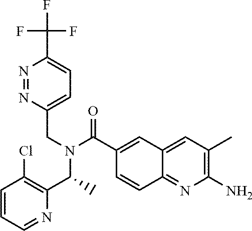 [SMILES: Cc1cc2cc(C(=O)N(Cc3ccc(C(F)(F)F)nn3)[C@H](C)c3ncccc3Cl)ccc2nc1N] | 2-amino-N-((1R)-1-(3-chloro-2-pyridinyl)ethyl)-3-methyl-N-((6-(trifluoromethyl)-3-pyridazinyl)methyl)-6-quinolinecarboxamide | 1st peak, Chiral Technologies AS column (250 × 21 mm, 5 mm) with a mobile phase of 85% liquid CO₂ and 15% MeOH with 0.2% TEA | 501.2 |
| 526 | 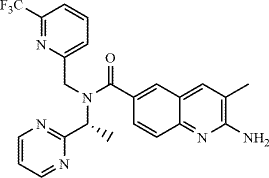 [SMILES: Cc1cc2cc(C(=O)N(Cc3cccc(C(F)(F)F)n3)[C@H](C)c3ncccn3)ccc2nc1N] | 2-amino-3-methyl-N-((1R)-1-(2-pyrimidinyl)ethyl)-N-((6-(trifluoromethyl)-2-pyridinyl)methyl)-6-quinolinecarboxamide | 1st peak, Chiral Technologies OD column (250 ×30 mm, 5 µm) with a mobile phase of 60% liquid CO₂ and 40% MeOH with 0.2% TEA. | 467.2 |
| 527 | 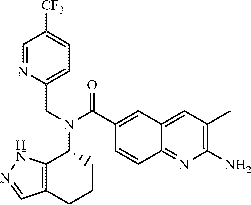 [SMILES: Cc1cc2cc(C(=O)N(Cc3ccc(C(F)(F)F)cn3)[C@@H]3CCCc4cn[nH]c43)ccc2nc1N] | 2-amino-3-methyl-N-((7R)-4,5,6,7-tetrahydro-1H-indazol-7-yl)-N-((5-(trifluoromethyl)-2-pyridinyl)methyl)-6-quinolinecarboxamide | 2nd peak, SFC using a Regis Whelk-01 (R,R) column (250 × 21 mm, 5 µm) with a mobile phase of 70% liquid CO₂ and 30% MeOH with 0.2% TEA. | 481.2 |
| 528 | 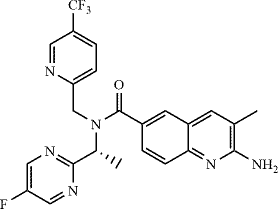 [SMILES: Cc1cc2cc(C(=O)N(Cc3ccc(C(F)(F)F)cn3)[C@H](C)c3ncc(F)cn3)ccc2nc1N] | 2-amino-N-((1R)-1-(5-fluoro-2-pyrimidinyl)ethyl)-3-methyl-N-((5-(trifluoromethyl)-2-pyridinyl)methyl)-6-quinolinecarboxamide | 1st Peak. Chiral Technologies AS column (250 × 21 mm, 5 mm) and AS (150 x 21 mm, 5 µm). Mobile phase: 85:15. B: 0.2% TEA in MeOH. | 485 |
| 529 | 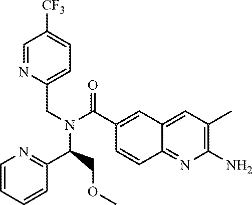 [SMILES: COC[C@@H](c1ccccn1)N(Cc1ccc(C(F)(F)F)cn1)C(=O)c1ccc2nc(N)c(C)cc2c1] | 2-amino-N-((1R)-2-methoxy-1-(2-pyridinyl)ethyl)-3-methyl-N-((5-(trifluoromethyl)-2-pyridinyl)methyl)-6-quinolinecarboxamide | 2nd Peak. Chiral Technologies OX (250 × 21 mm, 5 µm). Mobile phase: 70:30. B: 0.2% TEA in MeOH. | 496.2 |
| 530 | 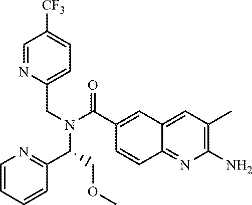 [SMILES: COC[C@H](c1ccccn1)N(Cc1ccc(C(F)(F)F)cn1)C(=O)c1ccc2nc(N)c(C)cc2c1] | 2-amino-N-((1S)-2-methoxy-1-(2-pyridinyl)ethyl)-3-methyl-N-((5-(trifluoromethyl)-2-pyridinyl)methyl)-6-quinolinecarboxamide | 1st Peak. Chiral Technologies OX (250 × 21 mm, 5 µm). Mobile phase: 70:30. B: 0.2% TEA in MeOH. | 496.2 |
| 531 | 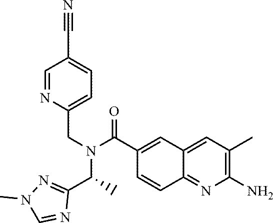 | 2-amino-N-((5-cyano-2-pyridinyl)methyl)-3-methyl-N-((lR)-1-(1-methyl-1H-1,2,4-triazol-3-yl)ethyl)-6-quinolinecarboxamide | 1st peak. Regis Whelk-O S,S 2 ×15 cm, 5 µm; mobile phase of 50% MeOH w/ 0.2% DEA | 427.2 |
| 532 | 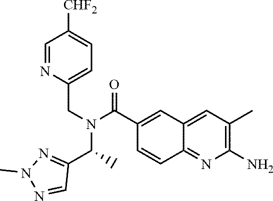 | 2-amino-N-((5-(difluoromethyl)-2-pyridinyl)methyl)-3-methyl-N-((lR)-1-(1-methyl-1H-1,2,4-triazol-3-yl)ethyl)-6-quinolinecarboxamide | 1st peak. Chiral Technologies AD (30 ×250 mm, 5 µm); Mobile Phase: 60:40; B: 0.2% TEA in EtOH. | 452.0 |
| 533 | 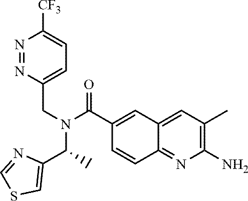 [SMILES: Cc1cc2cc(C(=O)N(Cc3ccc(C(F)(F)F)nn3)[C@H](C)c3cscn3)ccc2nc1N] | 2-amino-3-methyl-N-((1R)-1-(1,3-thiazol-4-yl)ethyl)-N-((6-(trifluoromethyl)-3-pyridazinyl)methyl)-6-quinolinecarboxamide | 1st peak. Chiral Technologies AS (30 ×150 mm, 5 µm); Mobile Phase: 80:20; B: 0.2% TEA in MeOH. | 473.2 |
| 534 | 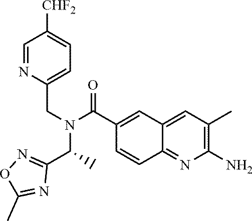 [SMILES: Cc1nc([C@@H](C)N(Cc2ccc(C(F)F)cn2)C(=O)c2ccc3nc(N)c(C)cc3c2)no1] | 2-amino-N-((5-(difluoromethyl)-2-pyridinyl)methyl)-3-methyl-N-((1R)-1-(5-methyl-1,2,4-oxadiazol-3-yl)ethyl)-6-quinolinecarboxamide | 1st peak. Regis (S,S) Whelk-01 column (50 × 21 mm, 5 µm); mobile phase of 35% MeOH w/ 0.2% DEA. | 453.2 |
| 535 | 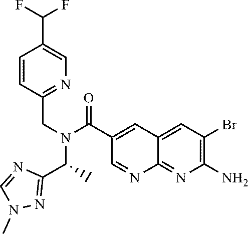 [SMILES: C[C@H](c1ncn(C)n1)N(Cc1ccc(C(F)F)cn1)C(=O)c1cnc2nc(N)c(Br)cc2c1] | 7-amino-6-bromo-N-((5-(difluoromethyl)-2-pyridinyl)methyl)-N-((1R)-1-(1-methyl-1H-1,2,4-triazol-3-yl)ethyl)-1,8-naphthyridine-3 -carboxamide | 1st Peak. Chiralcel OD-H (2 ×25 cm, 5 µm); Mobile phase: 65:35; B: 0.2% DEA in MeOH. | 517/519 |
| 536 | 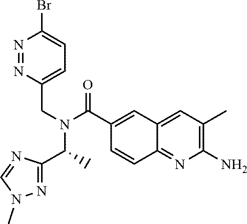 [SMILES: Cc1cc2cc(C(=O)N(Cc3ccc(Br)nn3)[C@H](C)c3ncn(C)n3)ccc2nc1N] | 2-amino-N-((6-bromo-3-pyridazinyl)methyl)-3-methyl-N-((lR)-1-(1-methyl-1H-1,2,4-triazol-3-yl)ethyl)-6-quinolinecarboxamide | 1st Peak. Chiralcel OD-H (2 ×25 cm, 5 µm); Mobile phase: 60:40; B: 0.2% DEA in MeOH. | 481/483 |
| 809 | 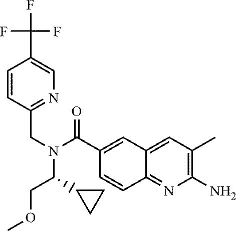 [SMILES: COC[C@@H](C1CC1)N(Cc1ccc(C(F)(F)F)cn1)C(=O)c1ccc2nc(N)c(C)cc2c1] | 2-amino-N-((1R)-1-cyclopropyl-2-methoxyethyl)-3-methyl-N-((5-(trifluoromethyl)-2-pyridinyl)methyl)-6-quinolinecarboxamide | 1st peak: Regis (S,S) Whelk-01 column (250 × 21 mm, 5 µm); mobile phase 50:50 MeOH with 0.2% TEA | 459.2 |
| 810 | 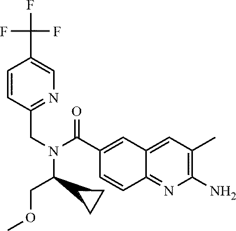 [SMILES: COC[C@H](C1CC1)N(Cc1ccc(C(F)(F)F)cn1)C(=O)c1ccc2nc(N)c(C)cc2c1] | 2-amino-N-((1S)-1-cyclopropyl-2-methoxyethyl)-3-methyl-N-((5-(trifluoromethyl)-2-pyridinyl)methyl)-6-quinolinecarboxamide | 2nd peak: Regis (S,S) Whelk-01 column (250 × 21 mm, 5 µm); mobile phase 50:50 MeOH with 0.2% TEA | 459.2 |
| 811 | | (R)-2-amino-N-(1-(6-cyanopyridin-2-yl)ethyl)-3-methyl-N-((5-(trifluoromethyl)pyridin-2-yl)methyl)quinoline-6-carboxamide | 1st Peak. Chiral Tech ID (250 ×21 cm, 5 µm); Mobile phase: 50:50; B: 0.2% TEA in IPA. | 491.1 |
| 812 | | 2-amino-N-((1R,2R)-2-cyanocyclopentyl)-3-methyl-N-((5 -(trifluoromethyl)pyridin-2-yl)methyl)quinoline-6-carboxamide | 2nd Peak. Chiral Tech IF (250 ×21 cm, 5 µm); Mobile phase: 50:50; B: 0.2% TEA in MeOH. | 454.1 |
| 813 | 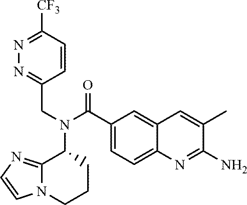 [SMILES: Cc1cc2cc(C(=O)N(Cc3ccc(C(F)(F)F)nn3)[C@@H]3CCCn4ccnc43)ccc2nc1N] | 2-amino-3-methyl-N-((8R)-5,6,7,8-tetrahydroimidazo[1,2-a]pyridin-8-yl)-N-((6-(trifluoromethyl)-3-pyridazinyl)methyl)-6-quinolinecarboxamide | 2nd peak. Chiral Technologies AS column (250 × 21 mm, 5 mm) with a mobile phase of 75% Liquid CO2 and 25% MeOH with 0.2% TEA using a flowrate of 80 mL/min. | 482.2 |
| 814 | 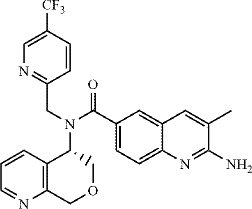 [SMILES: Cc1cc2cc(C(=O)N(Cc3ccc(C(F)(F)F)cn3)[C@@H]3COCc4ncccc43)ccc2nc1N] | (S)-2-amino-N-(5,8-dihydro-6H-pyrano[3,4-b]pyridin-5-yl)-3 -methyl-N-((5-(trifluoromethyl)pyridin-2-yl)methyl)quinoline-6-carboxamide | 1st peak. Chiral Technologies AD column (250 × 21 mm, 5 mm) with a mobile phase of 67% Liquid CO2 and | 494.2 |
| | | | 33% MeOH with 0.2% TEA using a flowrate of 80 mL/min. | |
| 815 | 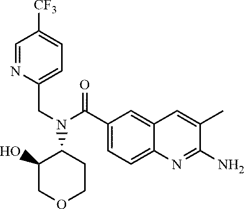 [SMILES: Cc1cc2cc(C(=O)N(Cc3ccc(C(F)(F)F)cn3)[C@@H]3CCOC[C@H]3O)ccc2nc1N] | 2-amino-N-((3 S,4R)-3-hydroxytetrahydro-2H-pyran-4-yl)-3-methyl-N-((5-(trifluoromethyl)-2-pyridinyl)methyl)-6-quinolinecarboxamide | 2nd peak. Regis (S,S) Whelk-01 column (250 × 21 mm, 5 mm) with a mobile phase of 50% Liquid CO2 and 50% MeOH with 0.2% TEA using a flowrate of 60 mL/min. | 461.2 |
| 816 | 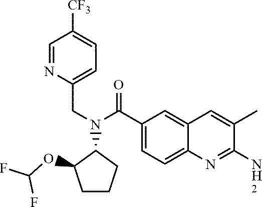 | 2-amino-N-((1R,2R)-2-(difluoromethoxy)cyclopentyl)-3-methyl-N-((5-(trifluoromethyl)pyridin-2-yl)methyl)quinoline-6-carboxamide | 1st Peak. Chiral Technologies IG column (250 × 21 mm, 5 µm) with a mobile phase of 55% Liquid CO2 and 45% MeOH with 0.2% TEA. | 495.2 |
| 817 | 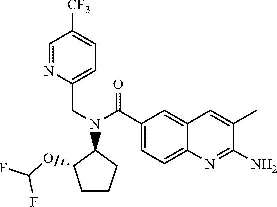 [SMILES: Cc1cc2cc(C(=O)N(Cc3ccc(C(F)(F)F)cn3)[C@H]3CCC[C@@H]3OC(F)F)ccc2nc1N] | 2-amino-N-((1 S,2S)-2-(difluoromethoxy)cyclopentyl)-3-methyl-N-((5-(trifluoromethyl)pyridin-2-yl)methyl)quinoline-6-carboxamide | 2nd peak. Chiral Technologies IG column (250 × 21 mm, 5 µm) with a mobile phase of 55% Liquid | 495.2 |
| | | | CO2 and 45% MeOH with 0.2% TEA. | |
| 818 | 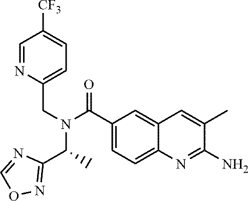 [SMILES: Cc1cc2cc(C(=O)N(Cc3ccc(C(F)(F)F)cn3)[C@H](C)c3ncon3)ccc2nc1N] | (R)-N-(1-(1 ,2,4-oxadiazol-3-yl)ethyl)-2-amino-3-methyl-N-((5-(trifluoromethyl)pyridin-2-yl)methyl)quinoline-6-carboxamide | Peak 1. Chiral Technologies IC column (250 × 21 mm, 5 µm) with a mobile phase of 60% Liquid CO2 and 40% MeOH with 0.2% TEA using a flowrate of 80 mL/min. | 457.1 |
| 819 | | 2-amino-N-((6-chloro-3-pyridazinyl)methyl)-3-methyl-N-((1R)-1-(2-pyrimidinyl)ethyl)-6-quinolinecarboxamide | Peak 2: Chiral Technologies AD column (250 × 21 mm, 5 mm) with a mobile phase of 85% Liquid CO2 and 15% MeOH with 1.0% TEA using a flowrate of 100 mL/min | 434.2 |
| 820 | | 2-amino-N-((6-cyano-2-methyl-3-pyridinyl)methyl)-3-methyl-N-((1R)-1-(2-pyrimidinyl)ethyl)-6-quinolinecarboxamide | 1st Peak: Chiralpak-AS, 5um, 21×250 mm column, with 30% MeOH+ 0.2% TEA using a flowrate of 80 | 438.2 |
| | | | mL/min (56 g/min | |

Example 537: 7-amino-N-((3-fluoropyridin-2-yl)methyl)-6-methyl-N-((5-(trifluoromethyl)pyridin-2-yl)methyl)-1,8-naphthyridine-3-carboxamide.

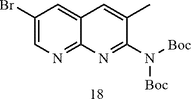

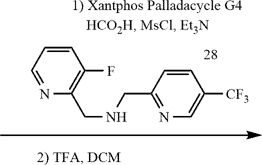

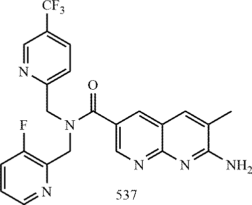

A COware system (Sigma-Aldrich) was used for this reaction. Chamber 1 was charged with: formic acid (33 mg, 0.72 mmol), methanesulfonyl chloride (83 mg, 0.72 mmol), triethylamine (0.20 mL, 1.44 mmol) in toluene (1 mL). Chamber 2 was charged with: methanesulfonato[9,9-dimethyl-4,5-bis(diphenylphosphino)xanthene] (2′-methylamino-1,1 ‘-biphenyl-2-yl)palladium(ii) (Xantphos Palladacycle G4) (17 mg, 0.018 mmol, Strem), 1-(3-fluoropyridin-2-yl)-N-((5-(trifluoromethyl)pyridin-2-yl)methyl)methanamine (28, 134 mg, 0.47 mmol), Intermediate 18 (158 mg, 0.36 mmol), triethylamine (0.20 mL, 1.44 mmol), and dioxane (2 mL). The COware system was heated at 60° C. for 18 h. The reaction mixture was allowed to cool to RT and filtered through a pad of celite, the solid was rinsed with 5 mL of EtOAc. The filtrate was concentrated to give a brown solid that contained m/z (ESI): 671.3 (M+H)⁺. The brown solid was suspended in DCM (2 mL) and treated with trifluoroacetic acid (2 mL). The reaction mixture was stirred at RT for 30 min then concentrated. The crude material was purified by reverse-phase preparative HPLC (using 0.1% TFA in CH₃CN/H₂O, gradient 10% to 100%0 to provide 7-amino-N-((3-fluoropyridin-2-yl)methyl)-6-methyl-N-((5-(trifluoromethyl)pyridin-2-yl)methyl)-1,8-naphthyridine-3-carboxamide 2,2,2-trifluoroacetate (537, 148 mg, 0.253 mmol, 70% yield) as an off-white solid. MS (ESI, +ve) m/z: 471 [M + H]⁺. ¹H NMR (400 MHz, DMSO-d₆) δ ppm 8.74 - 9.06 (m, 2 H), 8.38 - 8.65 (m, 4 H), 8.18 (br d, J=5.23 Hz, 2 H), 7.54 - 7.79 (m, 2 H), 7.43 (dt, J=8.47, 4.34 Hz, 1 H), 4.87 (br d, J=5.43 Hz, 3 H), 3.44 - 4.21 (m, 2 H), 2.31 (s, 3 H).

Example 538: 7-amino-6-methyl-N-((1R)-1-(2-pyrazinyl)ethyl)-N-((5-(trifluoromethyl)-2-pyridinyl)methyl)-1,8-naphthyridine-3-carboxamide.

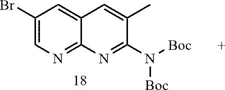

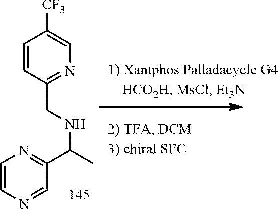

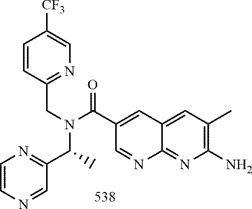

The title compound (538) was prepared from amine 145 in a manner similar to that described for Example 537, followed by chiral SFC (1^{st} eluting peak). MS (ESI, +ve) m/z: 468.1 [M + H]⁺. SFC conditions: Chiral Technology AD (250 x 21 mm, 5 µm). Mobile phase: 80:20. B: 0.2% TEA in MeOH.

Example 539: 2-amino-N-isobutyl-3,7-dimethyl-N-((5-(trifluoromethyl)pyridin-2-yl)methyl)quinoline-6-carboxamide.

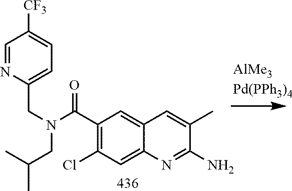

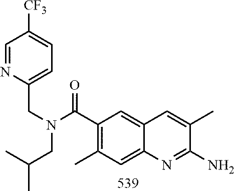

Trimethylaluminum (0.17 mL of 2.0 M solution in toluene, 0.34 mmol, Sigma-Aldrich) was added to a stirred mixture of 2-amino-7-chloro-N-isobutyl-3-methyl-N-((5-(trifluoromethyl)pyridin-2-yl)methyl)quinoline-6-carboxamide (436, 77 mg, 0.17 mmol) and tetrakis(triphenylphosphine)palladium (39.5 mg, 0.034 mmol, Sigma-Aldrich) in 1,4-dioxane (0.5 mL) in a microwave vial under a nitrogen atmosphere. The reaction mixture was heated in a microwave at 130° C. for 6 h then cooled to RT and partitioned between EtOAc (40 mL) and saturated NaHCO₃ (40 mL). The organic layer was separated, washed with brine (40 mL), dried over MgSO₄, filtered, and concentrated in vacuo. Chromatographic purification of the residue (silica gel, 0% to 100% EtOAc in heptane) gave the title compound (539, 4 mg, 5% yield) as a light-yellow solid. MS (ESI, +ve) m/z: 431.1 [M + H]⁺.

Example 540: (R)-2-amino-N-((5-(cyclopropylsulfonyl)pyridin-2-yl)methyl)-N-(1-(3-fluoropyridin-2-yl)ethyl)-3-methylquinoline-6-carboxamide.

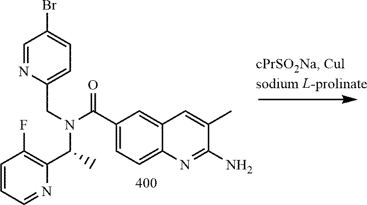

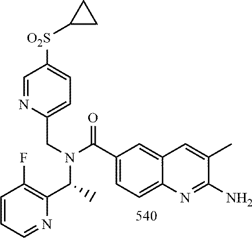

To a mixture of (R)-2-amino-N-((5-bromopyridin-2-yl)methyl)-N-(1-(3-fluoropyridin-2-yl)ethyl)-3-methylquinoline-6-carboxamide (400, 45 mg, 0.09 mmol), sodium cyclopropanesulfinate (46 mg, 0.36 mmol, Accela ChemBio Inc.), (S)-pyrrolidine-2-carboxylic acid, sodium salt (10 mg, 0.07 mmol, Combi-Blocks), and copper(i) iodide (7 mg, 0.03 mmol, Sigma-Aldrich) in a 5-mL microwave vessel was added dimethyl sulfoxide (3.0 mL). The resulting solution was purged with nitrogen for 10 min before sealed and heated in a microwave for 3 h at 95° C. The mixture was purified via reverse-phase HPLC (10% to 85% MeCN in water mediated with 0.1% TFA) to give, after lyophilization, (R)-2-amino-N-((5-(cyclopropylsulfonyl)pyridin-2-yl)methyl)-N-(1-(3-fluoropyridin-2-yl)ethyl)-3-methylquinoline-6-carboxamide TFA salt (26 mg, 0.041 mmol, 45% yield) as a white solid. m/z (ESI): 520.0 (M+H)⁺. ¹H NMR (400 MHz, METHANOL-d₄) δ 8.82 (dd, J=0.63, 2.30 Hz, 1H), 7.88-8.39 (m, 5H), 7.73-7.87 (m, 1H), 7.36-7.66 (m, 2H), 7.28 (td, J=4.34, 8.47 Hz, 1H), 5.02-5.57 (m, 1H), 4.88-4.95 (m, 2H), 2.72 (br s, 1H), 2.28-2.51 (m, 3H), 1.60-1.80 (m, 3H), 1.22-1.31 (m, 2H), 1.05-1.15 (m, 2H). ¹⁹F NMR (376 MHz, METHANOL-d₄) δ -77.09 (s, 3F from TFA), -125.00 (s, 1F). m/z (ESI): 520.0 (M+H)⁺.

Example 541: (R)-2-amino-N-((5-(methylsulfonyl)pyridin-2-yl)methyl)-N-(1-(3-fluoropyridin-2-yl)ethyl)-3-methylquinoline-6-carboxamide. This molecule was prepared in a manner similar to that described for Example 540. m/z (ESI): 494.1 (M+H)⁺.

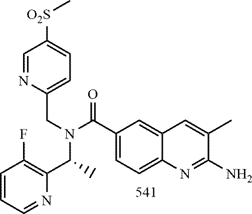

Example 542: (R)-2-amino-N-((5-cyanopyridin-2-yl)methyl)-N-(1-(3-fluoropyridin-2-yl)ethyl)-3-methylquinoline-6-carboxamide.

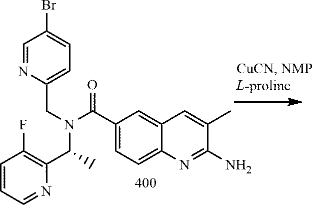

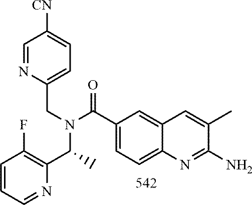

A mixture of (R)-2-amino-N-((5-bromopyridin-2-yl)methyl)-N-(1-(3-fluoropyridin-2-yl)ethyl)-3-methylquinoline-6-carboxamide (400, 100 mg, 0.20 mmol), L-(-)-proline (23 mg, 0.20 mmol), and copper (i) cyanide (45 mg, 0.50 mmol) in NMP (2.0 mL) in a 5-mL microwave vessel was purged with nitrogen for 10 min before sealed and subjected to microwave heating (2 h at 140° C.). The mixture was treated with CuCN (~ 25 mg) then heated in a microwave (16 h at 140° C.). The crude reaction mixture was filtered and the filtrate was purified via reverse-phase HPLC (10% to 85% MeCN in water mediated by 0.1% TFA) to give, after lyophilization, (R)-2-amino-N-((5-cyanopyridin-2-yl)methyl)-N-(1-(3-fluoropyridin-2-yl)ethyl)-3-methylquinoline-6-carboxamide (10 mg) as an off-white solid as a free base (Note that no TFA signal was observed ¹⁹ F NMR). m/z (ESI): 441.2 (M+H)⁺. ¹H NMR (400 MHz, METHANOL-d₄) δ 8.72 (br s, 1H), 8.15-8.33 (m, 2H), 7.69-8.10 (m, 4H), 7.23-7.51 (m, 3H), 5.40-6.51 (m, 1H), 4.88-4.94 (m, 2H), 2.40 (br s, 3H), 1.70 (br s, 3H). ¹⁹F NMR (376 MHz, METHANOL-d₄) δ -125.09 (s, 1F).

Example 543: (R)-7-amino-6-ethynyl-N-(1-(3-fluoropyridin-2-yl)ethyl)-N-((5-(trifluoromethyl)pyridin-2-yl)methyl)-1,8-naphthyridine-3-carboxamide.

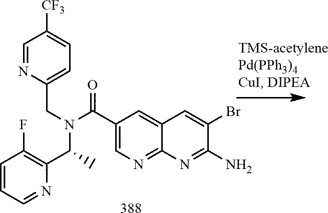

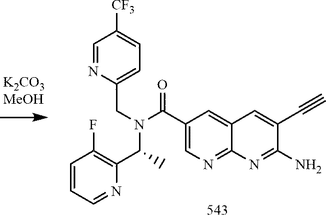

A mixture of (R)-7-amino-6-bromo-N-(1-(3-fluoropyridin-2-yl)ethyl)-N-((5-(trifluoromethyl)pyridin-2-yl)methyl)-1,8-naphthyridine-3-carboxamide 2,2,2-trifluoroacetate (388, 100 mg, 0.15 mmol), Pd(PPh₃)₄ (35 mg, 0.03 mmol, Combi Blocks), copper(i) iodide (11 mg, 0.06 mmol), and 1,1′-dimethyltriethylamine (0.10 mL, 0.60 mmol) in NMP (2.5 mL) in a 5-mL microwave reaction vessel was purged with nitrogen for 10 min before (trimethylsilyl)acetylene (0.11 mL, 0.75 mmol, Matrix Scientific) was introduced. The vessel was sealed and subjected to microwave heating (15 h at 70° C.). The crude was directly loaded onto a silica gel column and eluted with MeOH (with 0.5% ammonium hydroxide)/DCM to give an impure mixture of (R)-7-amino-N-(1-(3-fluoropyridin-2-yl)ethyl)-N-((5-(trifluoromethyl)pyridin-2-yl)methyl)-6-((trimethylsilyl)ethynyl)-1,8-naphthyridine-3-carboxamide and (R)-7-amino-6-ethynyl-N-(1-(3-fluoropyridin-2-yl)ethyl)-N-((5-(trifluoromethyl)pyridin-2-yl)methyl)-1,8-naphthyridine-3-carboxamide. To this was added MeOH (5 mL) followed by potassium carbonate (98 mg, 0.70 mmol) and the resulting mixture was stirred at RT for 20 min. The crude was directly loaded onto a silica gel column and eluted with MeOH (with 0.5% ammonium hydroxide)/DCM to give an impure desired product, which was dissolved in MeOH and subjected to preparative reverse-phase HPLC (10% to 85% MeCN in water mediated by 0.1% TFA) to give the title compound as a TFA salt (543, 9.5 mg) as a white solid. m/z (ESI): 495.0 (M+H)⁺. ¹H NMR (400 MHz, METHANOL-d4) δ 8.97 (br s, 1H), 8.25-8.81 (m, 3H), 7.73-8.08 (m, 1H), 7.08-7.53 (m, 3H), 5.43-6.49 (m, 1H), 4.89-5.00 (m, 3H), 4.34 (br s, 1H), 1.53-1.87 (m, 3H).

Example 544: (R)-2-amino-3-iodo-N-(1-(pyrimidin-2-yl)ethyl)-N-((5-(trifluoromethyl)pyridin-2-yl)methyl)quinoline-6-carboxamide.

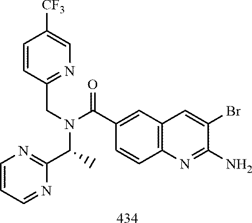

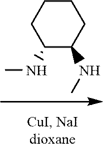

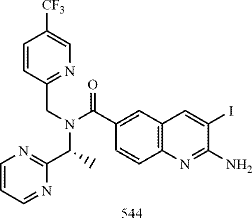

A mixture trans-(-)-N,N″-dimethylcyclohexane-1,2-diamine (4.02 mg, 0.028 mmol, Sigma-Aldrich), copper(i) iodide (2.69 mg, 0.014 mmol, Acros Organics), sodium iodide (52.9 mg, 0.353 mmol, Sigma-Aldrich), and (R)-2-amino-3-bromo-N-(1-(pyrimidin-2-yl)ethyl)-N-((5-(trifluoromethyl)pyridin-2-yl)methyl)quinoline-6-carboxamide (434, 75 mg, 0.14 mmol) in 2 mL of dioxane was heated at 125° C. for 8 h. The mixture was diluted with 50 mL of EtOAc and filtered through a pad of Celite. The filtrate was washed with 5 mL of water. The organic solution was concentrated and the residue was purified on a silica gel column (1% to 5% MeOH in EtOAc) to provide a brown solid that contained a mixture of 434 : 544 = 2:3. This material (80 mg) was purified by SFC using a Regis (S,S) Whelk-01 column (250 × 21 mm, 5 µm) with a mobile phase of 50% liquid CO₂ and 50% MeOH with 0.2% TEA using a flowrate of 60 mL/min, to generate 14 mg of peak 1 (434, m/z (ESI): 531/533 (M+H)⁺) and 20 mg of peak 2 (544, m/z (ESI): 578.9 (M+H)⁺). (R)-2-Amino-3-iodo-N-(1-(pyrimidin-2-yl)ethyl)-N-((5-(trifluoromethyl)pyridin-2-yl)methyl)quinoline-6-carboxamide (544, 20 mg, 24% yield) was obtained as a brown solid. ¹H NMR (METHANOL-d₄, 400 MHz) δ 8.75 (m, 3H), 8.63 (s, 1H), 8.0-8.1 (m, 1H), 7.92 (m, 1H), 7.7-7.8 (m, 1H), 7.5-7.7 (m, 2H), 7.34 (t, 1H, J=4.9 Hz), 5.91 (m, 0.3H), 5.49 (m, 0.7H), 4.9-5.1 (m, 1H), 4.7-4.8 (m, 1H), 1.71 (d, 3H, J=7.0 Hz).

Example 545: 2-amino-N-((5-cyano-2-pyridinyl)methyl)-3,4-dimethyl-N-((1R)-1-(2-pyrimidinyl)ethyl)-6-quinolinecarboxamide.

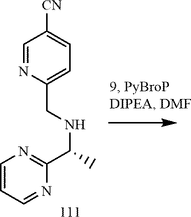

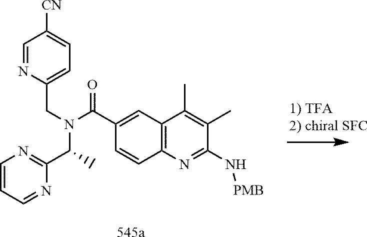

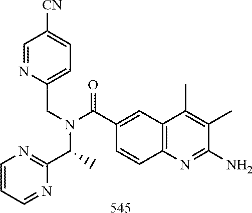

A mixture of (R)-6-(((1-(pyrimidin-2-yl)ethyl)amino)methyl)nicotinonitrile (111, 88 mg, 0.368 mmol) and 2-((4-methoxybenzyl)amino)-3,4-dimethylquinoline-6-carboxylic acid hydrochloride (9, 91 mg, 0.245 mmol) in DMF (2.5 mL) at RT was treated with diisopropylethylamine (0.43 mL, 2.45 mmol) followed by PyBroP (286 mg, 0.61 mmol). The reaction mixture was heated at 70° C. for 3 h. It was cooled to RT, quenched with saturated NaHCO₃ (15 mL) and extracted with EtOAc (3 x 15 mL). The organic solution was dried over MgSO₄, filtered, and concentrated. The residue was purified by silica gel chromatography (0% to 100% EtOAc in heptane) to provide 176 mg of brown oil as a mixture of 545a (m/z (ESI): 558.2 (M+H)⁺) and tri(pyrrolidin-1-yl)phosphine oxide (m/z (ESI): 258.1 (M+H)⁺). The resulting brown oil was treated with trifluoroacetic acid (1.8 mL) then heated at 70° C. for 2.5 h. The mixture was concentrated in vacuo. The remaining orange solid was purified via silica gel chromatography to afford 30 mg of an off-white solid that had m/z (ESI): 438.2 (M+H)⁺. ¹H NMR (400 MHz, METHANOL-d4) δ ppm 8.78 (d, J=5.0 Hz, 3 H), 8.23 - 8.48 (m, 1 H), 8.09 (br d, J=7.9 Hz, 1 H), 7.47 - 7.98 (m, 3 H), 7.38 (t, J=4.9 Hz, 1 H), 5.33 - 5.95 (m, 1 H), 4.90 - 5.20 (m, 2 H), 2.46 - 2.78 (m, 3 H), 2.36 (br s, 3 H), 1.70 (d, J=7.1 Hz, 3 H). The ee of the isolated product was then assessed and it was found that the compound underwent epimerization during the deprotection with TFA. This material was subjected to chiral SFC to provide 2-amino-N-((5-cyano-2-pyridinyl)methyl)-3,4-dimethyl-N-((1R)-1-(2-pyrimidinyl)ethyl)-6-quinolinecarboxamide (545, 9 mg, m/z (ESI): 438.2 (M+H)⁺.) as the 1^{st} eluting peak. SFC conditions: Sample was purified via preparative SFC using a Chiral Technologies OD column (250 X 21 mm, 5 mm) with a mobile phase of 80% liquid CO₂ and 20% MeOH with 0.2% TEA using a flowrate of 80 mL/min. to generate 9 mg of peak 1 with an ee of >99% and 16.5 mg of peak 2 with an ee of 96.74%. Peak assignment determined by SFC with OD column with 20% MeOH with 0.2% TEA.

Example 546: 2-amino-N-((5-cyclopropyl-2-pyridinyl)methyl)-3-methyl-N-(2-methylpropyl)-6-quinolinecarboxamide.

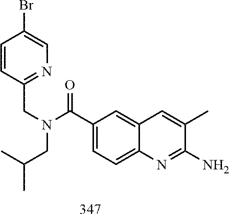

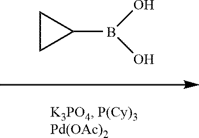

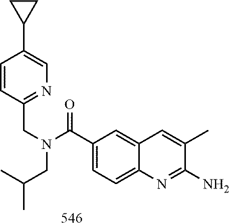

A mixture of 2-amino-N-((5-bromopyridin-2-yl)methyl)-N-isobutyl-3-methylquinoline-6-carboxamide (357, 23 mg, 0.054 mmol), cyclopropylboronic acid (23 mg, 0.269 mmol, Oakwood) and toluene (2 mL) was purged with argon, then potassium phosphate hydrate (37 mg, 0.161 mmol, Aldrich) and water (0.22 mL) were added. The mixture was stirred for 10 min at RT, then tricyclohexylphosphine (2 mg, 5.4 µmol, Strem) and palladium(II) acetate (0.6 mg, 2.7 µmol, Aldrich) were added. The mixture was stirred in a sealed vial at 90° C. overnight then filtered through celite. The filtrate was concentrated in vacuo. The crude material was purified by silica gel chromatography (0% to 100% (3/1 EtOAc/EtOH) in heptane to afford the title compound (546, 11 mg, 52% yield) as an off-white solid. MS: m/z (ESI): 389.1 (M+H)⁺. ¹H NMR (400 MHz, CHLOROFORM-d) δ ppm 8.27 - 8.43 (m, 1 H), 7.26 (s, 6 H), 5.04 (br s, 2 H), 4.56 - 4.92 (m, 2 H), 3.07 - 3.44 (m, 2 H), 2.29 (br s, 3 H), 1.82 - 1.99 (m, 2 H), 0.90 - 1.08 (m, 5 H), 0.71 (br s, 5 H).

Example 821: (R)-2-amino-N-((5-cyclopropylpyridin-2-yl)methyl)-3-methyl-N-(1-(pyrimidin-2-yl)ethyl)quinoline-6-carboxamide.

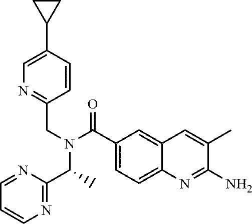 [SMILES: Cc1cc2cc(C(=O)N(Cc3ccc(C4CC4)cn3)[C@H](C)c3ncccn3)ccc2nc1N]

This molecule was prepared in a manner similar to that described for Example 546 from example 402. m/z (ESI): 439 (M+H)⁺.

Example 548. N-((1′-acetyl-1’,2’,3’,6′-tetrahydro[3,4′-bipyridin]-6-yl)methyl)-2-amino-3-methyl-N-((1R)-1-(2-pyrimidinyl)ethyl)-6-quinolinecarboxamide.

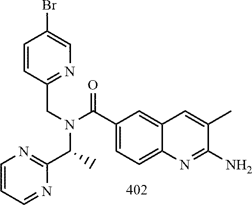

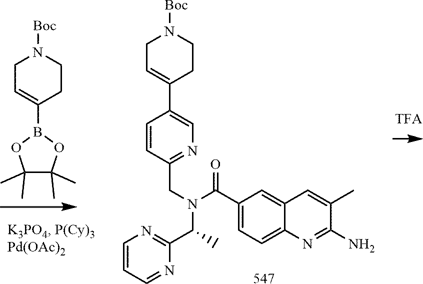

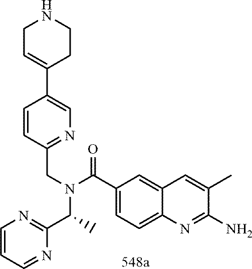

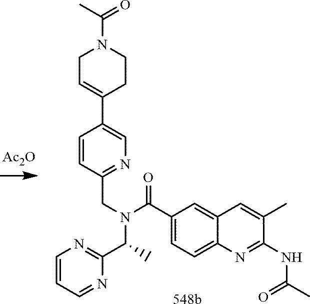

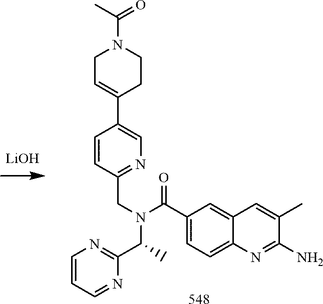

tert-Butyl (R)-6-((2-amino-3-methyl-N-(1-(pyrimidin-2-yl)ethyl)quinoline-6-carboxamido)methyl)-3’,6′-dihydro-[3,4′-bipyridine]-r(2’H)-carboxylate (547, MS: m/z (ESI): 580 (M+H)⁺) was prepared from Example 402 in a manner similar to that described for Example 546.

A mixture of tert-butyl (R)-6-((2-amino-3-methyl-N-(1-(pyrimidin-2-yl)ethyl)quinoline-6-carboxamido)methyl)-3’,6′-dihydro-[3,4′-bipyridine]-1′(2’H)-carboxylate (547, 34 mg, 0.059 mmol), DCM (1 mL), and TFA (0.5 mL) was stirred for 30 min at RT then concentrated in vacuo to give 548a as an off-white solid, which was used in next step without purification. MS: m/z (ESI): 480 (M+H)⁺.

A mixture of 548a (0.031 g, 0.052 mmol), dichloromethane (2 mL), TEA (0.102 mL, 0.731 mmol) and acetic anhydride (0.044 mL, 0.470 mmol) was stirred at RT for 18 h. The mixture was purified by silica gel chromatography (0% to 100%, then 100% EtOAc/EtOH (3/1) in heptane) to give (R)-2-acetamido-N-((1′-acetyl-1’,2’,3’,6′-tetrahydro-[3,4′-bipyridin]-6-yl)methyl)-3-methyl-N-(1-(pyrimidin-2-yl)ethyl)quinoline-6-carboxamide (548b, 29 mg) as off-white solid. MS: m/z (ESI): 564 (M+H)⁺.

A mixture of 548b (0.029 g, 0.051 mmol), lithium hydroxide hydrate (0.017 g, 0.412 mmol), MeOH (3 mL), and water (1 mL) was stirred at RT for 18 h. The mixture was purified by reverse phase HPLC (10-70% water in MeCN with 0.1% TFA) to give N-((1′-acetyl-1’,2’,3’,6′-tetrahydro[3,4′-bipyridin]-6-yl)methyl)-2-amino-3-methyl-N-((1R)-1-(2-pyrimidinyl)ethyl)-6-quinolinecarboxamide TFA salt (548, 30 mg, 925 yield) as a white solid. MS: m/z (ESI): 522 (M+H)⁺. ¹H NMR (400 MHz, METHANOL-d₄) δ ppm 8.80 (d, J=5.0 Hz, 2 H), 8.63 - 8.72 (m, 1 H), 7.54 - 8.28 (m, 6 H), 7.39 (t, J=5.0 Hz, 1 H), 6.32 - 6.42 (m, 1 H), 5.35 - 5.44 (m, 1 H), 5.02 (br d, J=15.1 Hz, 1 H), 4.79 (br d, J=3.7 Hz, 1 H), 4.26 (br s, 2 H), 3.81 (dt, J=20.0, 5.7 Hz, 2 H), 2.52 - 2.71 (m, 2 H), 2.40 (br s, 3 H), 2.17 (d, J=15.3 Hz, 3 H), 1.73 (br d, J=7.0 Hz, 3 H).

Example 549: 2-amino-N-((5-cyano-3-methyl-2-pyridinyl)methyl)-3-methyl-N-((1R)-1-(2-pyrimidinyl)ethyl)-6-quinolinecarboxamide.

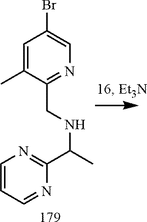

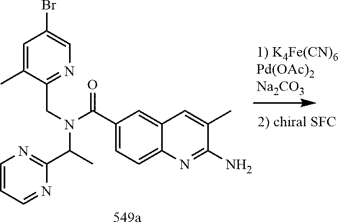

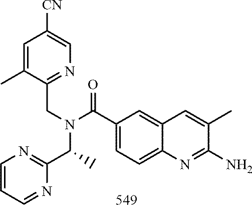

2-Amino-N-((5-bromo-3-methylpyridin-2-yl)methyl)-3-methyl-N-(1-(pyrimidin-2-yl)ethyl)quinoline-6-carboxamide (549a, MS: m/z (ESI): 491/493 (M+H)⁺) was prepared from amine 179 and acid chloride 16 according to Method B.

A mixture of 2-amino-N-((5-bromo-3-methylpyridin-2-yl)methyl)-3-methyl-N-(1-(pyrimidin-2-yl)ethyl)quinoline-6-carboxamide hydrochloride (549a, 0.14 g, 0.26 mmol), sodium carbonate (0.10 g, 0.92 mmol), N, N-dimethylacetamide (2 mL), potassium ferrocyanide trihydrate (0.11 g, 0.26 mmol, Toronto Research Chemicals), and palladium acetate (6 mg, 0.026 mmol, Strem) was purged with Ar then stirred at 120° C. overnight. The mixture was filtered through celite. The filtrate was purified by reverse phase HPLC (10-70% water in MeCN with 0.1% TFA) to give 2-amino-N-((5-cyano-3-methylpyridin-2-yl)methyl)-3-methyl-N-(1-(pyrimidin-2-yl)ethyl)quinoline-6-carboxamide as a TFA salt (59 mg) as a grey solid. MS: m/z (ESI): 438 (M+H)⁺.

The racemic material (59 mg) was subjected to chiral SFC using a Chiral Technologies OD column (250 × 21 mm, 5 µm) with a mobile phase of 70% liquid CO₂ and 30% EtOH with 0.2% TEA using a flowrate of 70 mL/min. The more potent (measured by IC₅₀ in HCT116 MTAP null cell viability assay) enantiomer was assigned as the (R)-; the less potent (measured by IC₅₀ in HCT116 MTAP null cell viability assay) enantiomer was assigned as (S)-. The 1^{st} eluting peak was 2-amino-N-((5-cyano-3-methyl-2-pyridinyl)methyl)-3-methyl-N-((1R)-1-(2-pyrimidinyl)ethyl)-6-quinolinecarboxamide (549, 20 mg, > 99% ee). MS: m/z (ESI): 438 (M+H)⁺. ¹H NMR (400 MHz, METHANOL-d₄) δ ppm 8.52 - 8.80 (m, 3 H), 7.20 - 7.98 (m, 6 H), 5.40 - 5.56 (m, 1 H), 5.01 - 5.17 (m, 1 H), 4.65 - 4.76 (m, 1 H), 1.94 - 2.49 (m, 6 H), 1.63 - 1.70 (m, 3 H). The 2^{nd} eluting peak was 2-amino-N-((5-cyano-3-methyl-2-pyridinyl)methyl)-3-methyl-N-((1S)-1-(2-pyrimidinyl)ethyl)-6-quinolinecarboxamide (31 mg, > 99% ee).

Example 550: 2-amino-N-((5-carbamoyl-2-pyridinyl)methyl)-N-((1R)-1-(2-fluorophenyl)ethyl)-3-methyl-6-quinolinecarboxamide.

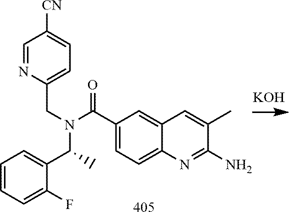

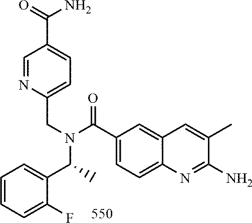

A mixture of (R)-2-amino-N-((5-cyanopyridin-2-yl)methyl)-N-(1-(2-fluorophenyl)ethyl)-3-methylquinoline-6-carboxamide 2,2,2-trifluoroacetate (405, 6 mg), tert-butanol (1 mL) and KOH (5 mg) was stirred at 85° C. for 55 min. The mixture was dissolved in MeOH/DMF, filtered and purified by reverse phase HPLC (10-70% water in MeCN with 0.1% TFA) to give the title compound as a TFA salt (3.2 mg, 51% yield) as a white solid. MS: m/z (ESI): 458 (M+H)⁺. ¹H NMR (400 MHz, METHANOL-d₄) δ ppm 8.85 (d, J=1.7 Hz, 1 H), 6.84 - 8.38 (m, 10 H), 5.32 - 5.44 (m, 1 H), 4.93 (s, 1 H), 4.78 (s, 1 H), 2.31 - 2.50 (m, 3 H), 1.59 - 1.79 (m, 3 H).

Example 551: methyl 6-((((2-amino-3-methyl-6-quinolinyl)carbonyl)((1R)-1-(2-pyrimidinyl)ethyl)amino)methyl)-3’,6′-dihydro[3,4′-bipyridine]-1′(2′H)-carboxylate.

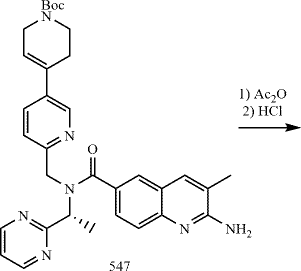

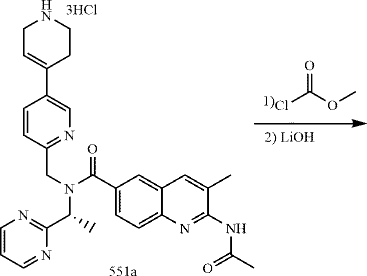

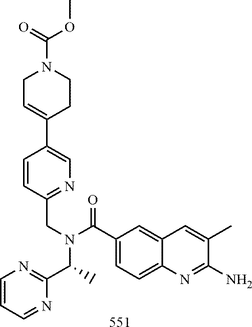

A mixture of 547 (0.50 g, 0.85 mmol), dichloromethane (5 mL), N,N-diisopropylethylamine (0.60 mL, 3.42 mmol) and acetic anhydride (0.16 mL, 1.71 mmol) was stirred at RT for 18 h. The mixture was purified by silica gel chromatography (0% to 100% EtOAc/EtOH (3/1) in DCM) to afford tert-butyl (R)-6-((2-acetamido-3-methyl-N-(1-(pyrimidin-2-yl)ethyl)quinoline-6-carboxamido)methyl)-3’,6′-dihydro-[3,4′-bipyridine]-1′(2′H)-carboxylate (0.53 g) as an off-white solid. MS: m/z (ESI): 622 (M+H)⁺. To the off-white solid (0.53 g, 0.86 mmol) in DCM (10 mL) and MeOH (1 mL) was added HCl (4.0 M in dioxane, 1.93 mL). After stirring at RT for 18 h, the mixture was concentrated in vacuo to provide 551a as an orange solid, which was used as crude in next step. MS: m/z (ESI): 522 (M+H)⁺.

To a mixture of (R)-2-acetamido-3-methyl-N-(1-(pyrimidin-2-yl)ethyl)-N-((1’,2’,3’,6′-tetrahydro-[3,4′-bipyridin]-6-yl)methyl)quinoline-6-carboxamide trihydrochloride (551a, 0.094 g, 0.15 mmol,) in dichloromethane (2 mL) was added DIPEA (0.21 mL, 1.19 mmol) and methyl chloroformate (0.015 mL, 0.194 mmol, Aldrich). The mixture was stirred at rt overnight then concentrated in vacuo. The residue was purified by silica gel chromatography (0% to 100% EtOAc/EtOH (3/1) in DCM to provide methyl (R)-6-((2-acetamido-3-methyl-N-(1-(pyrimidin-2-yl)ethyl)quinoline-6-carboxamido)methyl)-3’,6′-dihydro-[3,4′-bipyridine]-1′(2′H)-carboxylate (90 mg) as an orange solid. MS: m/z (ESI): 580 (M+H)⁺. The orange solid (90 mg) was dissolved in MeOH (3 mL) and water (1 mL) and treated with lithium hydroxide hydrate (62 mg). After stirring for 18 h at RT, the mixture was purified by silica gel chromatography (05 to 100%, then 100% EtOAc/EtOH (3/1) in DCM) to give methyl (R)-6-((2-amino-3-methyl-N-(1-(pyrimidin-2-yl)ethyl)quinoline-6-carboxamido)methyl)-3’,6′-dihydro-[3,4′-bipyridine]-1′(2’H)-carboxylate (551, 24 mg, 30% yield) as a white solid. MS: m/z (ESI): 538 (M+H)⁺. ¹H NMR (400 MHz, METHANOL-d₄) δ ppm 8.73 (br s, 2 H), 8.43 - 8.48 (m, 1 H), 7.46 - 7.95 (m, 5 H), 7.39 (br d, J=8.2 Hz, 1 H), 7.31 (br t, J=4.5 Hz, 1 H), 6.11 - 6.25 (m, 1 H), 5.44 - 5.54 (m, 1 H), 4.94 - 5.05 (m, 1 H), 4.54 - 4.67 (m, 1 H), 4.11 - 4.17 (m, 2 H), 3.74 (s, 3 H), 3.70 (t, J=5.7 Hz, 2 H), 2.54 (br s, 2 H), 2.30 (br s, 3 H), 1.67 (br d, J=6.1 Hz, 3 H).

Example 552: N-((5-(1-acetyl-4-piperidinyl)-2-pyridinyl)methyl)-2-amino-3-methyl-N-((1R)-1-(2-pyrimidinyl)ethyl)-6-quinolinecarboxamide.

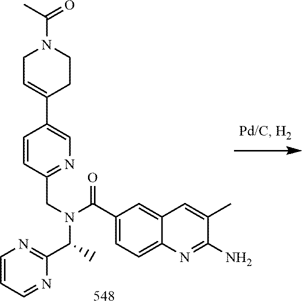

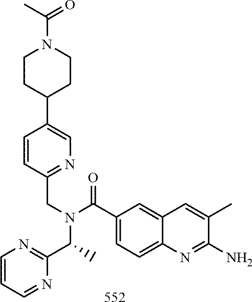

A mixture of 10% palladium on carbon (0.024 g, 0.023 mmol, Aldrich), 548 (0.059 g, 0.11 mmol,) and EtOH (3 mL) was hydrogenated with H₂ at 40 psi for 18 h. The mixture was filtered through celite. The celite was washed with 3/1 EtOAc/EtOH. The filtrate was concentrated in vacuo. The crude was purified by silica gel chromatography (1% to 10% MeOH/DCM with 1% concentrated NH₄OH in water) to give the title compound (552, 22 mg, 37% yield) as a white solid. MS: m/z (ESI): 524 (M+H)⁺. H-NMR (MeOH-d4): 31H were observed. ¹H NMR (400 MHz, METHANOL-d₄) δ ppm 8.65 - 8.81 (m, 2 H), 8.30 (s, 1 H), 7.24 - 7.99 (m, 7 H), 5.49 (s, 1 H), 4.91 - 5.04 (m, 1 H), 4.69 (br d, J=12.6 Hz, 1 H), 3.95 - 4.10 (m, 1 H), 3.88 (s, 1 H), 3.59 (s, 1 H), 3.19 - 3.28 (m, 1 H), 2.82 - 2.93 (m, 1 H), 2.66 - 2.76 (m, 1 H), 2.23 - 2.36 (m, 3 H), 2.14 (s, 3 H), 1.79 - 1.96 (m, 2 H), 1.54 - 1.75 (m, 4 H).

Example 822: \methyl (R)-4-(6-((2-amino-3-methyl-N-(1-(pyrimidin-2-yl)ethyl)quinoline-6-carboxamido)methyl)pyridin-3-yl)piperidine-1-carboxylate was prepared in a manner similar to that described for Example 552 using Example 551 as starting material. m/z (ESI): 540 (M+H)⁺.

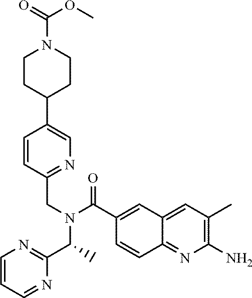 [SMILES: COC(=O)N1CCC(c2ccc(CN(C(=O)c3ccc4nc(N)c(C)cc4c3)[C@H](C)c3ncccn3)nc2)CC1]

Example 553: 2-amino-N-((5-cyano-2-pyrazinyl)methyl)-3-methyl-N-((1R)-1-(2-pyrimidinyl)ethyl)-6-quinolinecarboxamide.

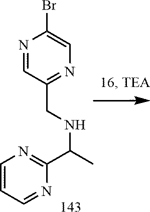

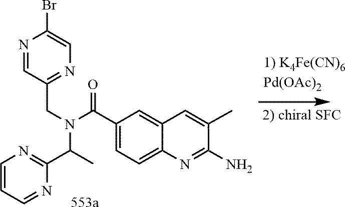

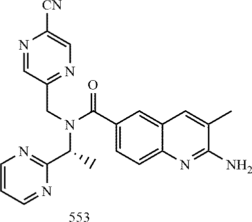

2-Amino-N-((5-bromopyrazin-2-yl)methyl)-3-methyl-N-(1-(pyrimidin-2-yl)ethyl)quinoline-6-carboxamide (553a, m/z (ESI): 478/480 (M+H)⁺) was prepared from amine 143 and acid chloride 16 according to Method B.

2-Amino-N-((5-cyano-2-pyrazinyl)methyl)-3-methyl-N-((1R)-1-(2-pyrimidinyl)ethyl)-6-quinolinecarboxamide (553, m/z (ESI): 425.1 (M+H)⁺) was prepared from amine 553a in a manner similar to that described for Example 549. Chiral SFC conditions: Chiral Technologies OD Column (250 X 21 mm, 5 µm); Mobile phase: 65:35; B: 0.2% TEA in MeOH). The 1^{st} eluting peak was Example 553 (> 99% ee). ¹H NMR (400 MHz, METHANOL-d₄) δ ppm 8.90 (d, J=1.5 Hz, 1 H), 8.84 (br s, 1 H), 8.76 (d, J=4.8 Hz, 2 H), 7.85 (br s, 2 H), 7.67 (br s, 1 H), 7.50 - 7.64 (m, 1 H), 7.37 (t, J=4.9 Hz, 1 H), 5.51 (br s, 1 H), 4.91 - 5.14 (m, 2 H), 2.32 (s, 3 H), 2.00 (s, 2 H), 1.76 (d, J=7.1 Hz, 3 H).

Example 554: (R)-2-amino-N-((6-(dimethylamino)pyridazin-3-yl)methyl)-3-methyl-N-(1-(pyrimidin-2-yl)ethyl)quinoline-6-carboxamide.

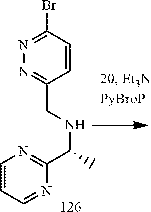

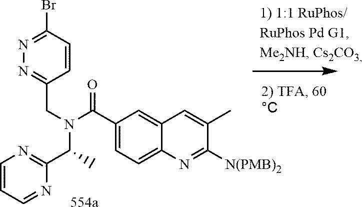

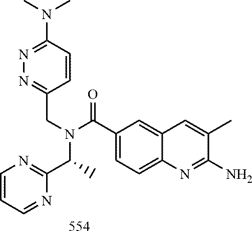

(R)-2-(bis(4-methoxybenzyl)amino)-N-((6-bromopyridazin-3-yl)methyl)-3-methyl-N-(1-(pyrimidin-2-yl)ethyl)quinoline-6-carboxamide (554a, m/z (ESI): 718.0, 720.0 (M+H)⁺) was prepared from amine 126 and acid 20 following the procedures described in Method A-PyBroP.

A mixture of RuPhos (18 mg, 0.039 mmol, Strem), RuPhos Pd G1 (32 mg, 0.39 mmol, Aldrich), (R)-2-(bis(4-methoxybenzyl)amino)-N-((6-bromopyridazin-3-yl)methyl)-3-methyl-N-(1-(pyrimidin-2-yl)ethyl)quinoline-6-carboxamide (554a, 110 mg, 015 mmol), dimethylamine (2.0 M in THF, 230 µL, 0.46 mmol, Aldrich) and cesium carbonate (200 mg, 0.62 mmol, Aldrich) in THF (1.0 mL) was heated at 85° C. for 2.5 h. The reaction mixture was filtered over a pad of diatomaceous earth and washed with EtOAc. The filtrate was concentrated and the crude product was purified by silica gel chromatography (0% to 100% EtOAc in heptane followed by 30% to 100% (3:1 EtOAc:EtOH):heptane) to give (R)-2-(bis(4-methoxybenzyl)amino)-N-((6-(dimethylamino)pyridazin-3-yl)methyl)-3-methyl-N-(1-(pyrimidin-2-yl)ethyl)quinoline-6-carboxamide (48 mg, 0.07 mmol, 45% yield). This material was dissolved in TFA (6.0 mL), heated to 60° C. for 18 h, and concentrated. The crude product was purified on an SCX column eluting with MeOH followed by 2 M ammonia in MeOH to give the title compound (554, 30 mg, 0.068 mmol, 44% yield) as a brown solid. m/z (ESI): 443.1 (M+H)⁺. ¹H NMR (400 MHz, METHANOL-d₄) δ ppm 8.71 (br d, J=4.2 Hz, 2 H), 7.82 (br s, 1 H), 7.75 (br s, 1 H), 7.64 (br s, 1 H), 7.54 (br d, J=7.5 Hz, 2 H), 7.29 (br t, J=4.8 Hz, 1 H), 7.02 (br d, J=8.2 Hz, 1 H), 5.26 - 5.77 (m, 1 H), 4.98 (br d, J=15.0 Hz, 1 H), 4.69 (br d, J=9.4 Hz, 1 H), 3.11 (s, 6 H), 2.27 (s, 3 H), 1.68 (br d, J=7.1 Hz, 3 H).

Example 555: (R)-2-amino-3-methyl-N-((6-morpholinopyridazin-3-yl)methyl)-N-(1-(pyrimidin-2-yl)ethyl)quinoline-6-carboxamide. This molecule was prepared in a manner similar to that described for Example 554. m/z (ESI): 485.1 (M+H)⁺.

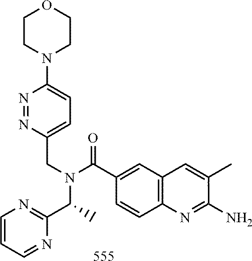

Example 823: 2-Amino-N-((1R)-1-(3-fluoro-2-pyridinyl)ethyl)-3-methyl-N-((6-(4-morpholinyl)-3-pyridazinyl)methyl)-6-quinolinecarboxamide. This molecule was prepared in a manner similar to that described for Example 554 from 205. m/z (ESI): 502.2 (M+H)⁺.

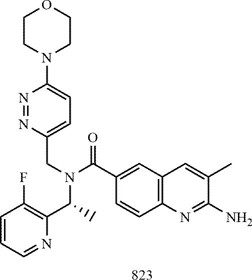

Example 824: 2-Amino-N-((1R)-1-(3-fluoro-2-pyridinyl)ethyl)-3-methyl-N-((6-(methylamino)-3-pyridazinyl)methyl)-6-quinolinecarboxamide. This molecule was prepared in a manner similar to that described for Example 554 from 205, except BrettPhos was used instead of RuPhos. m/z (ESI): 446.1 (M+H)⁺.

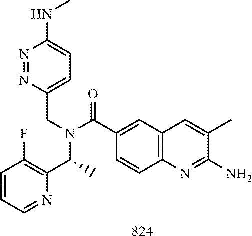

Example 556: (R)-N-((5-(2-oxa-6-azaspiro[3.3]heptan-6-yl)pyridin-2-yl)methyl)-2-amino-3-methyl-N-(1-(pyrimidin-2-yl)ethyl)quinoline-6-carboxamide.

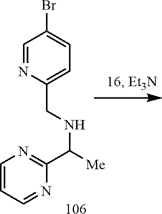

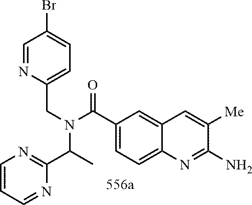

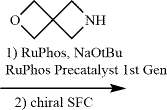

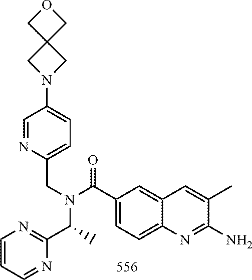

2-Amino-N-((5-bromopyridin-2-yl)methyl)-3-methyl-N-(1-(pyrimidin-2-yl)ethyl)quinoline-6-carboxamide (556a, m/z (ESI): 477/479 (M+H)⁺) was prepared from amine 106 and acid chloride 16 according to Method B.

To a stirred solution of dicycloheayl(2’,6′-diisopropoay-[1,1′-biphenyl]-2-yl)phosphane (26 mg, 0.057 mmol), chloro(2-dicyclohexylphosphino-2’,6′-di-i-propoxy-1,1′-biphenyl)[2-(2-aminocthylphenyl)] palladium (ii) methyl-t-butylether (46 mg, 0.057 mmol), sodium tert-butoxide (91 mg, 0.94 mmol) and 2-amino-N-((5-bromopyridin-2-yl)methyl)-3-methyl-N-(1-(pyrimidin-2-yl)ethyl)quinoline-6-carboxamide (556a, 180 mg, 0.37 mmol) in THF (3.8 mL) was added 2-oxa-6-azaspiro[3.3]heptane (37 mg, 0.37 mmol, Combi Blocks). The mixture was purged with N₂ for 10 min and then heated to 60° C. for 24 h. The reaction was cooled to RT, diluted with water (10 mL) and extracted with EtOAc (3 × 10 mL). The combined organics were concentrated and the residue was chromatographed on silica gel (0% to 100% (3:1 EtOAc/EtOH) in heptane) to give N-((5-(2-oxa-6-azaspiro[3.3]heptan-6-yl)pyridin-2-yl)methyl)-2-amino-3-methyl-N-(1-(pyrimidin-2-yl)ethyl)quinoline-6-carboxamide (42 mg, 0.085 mmol, 22.5% yield) as a brown solid. m/z (ESI): 496.3 (M+H)⁺. The racemic material was subjected to chiral SFC under the conditions indicated below to give 2 eluents. Chiral SFC: Chiral Technologies OJ column (250 × 21 mm, 5 µm) with a mobile phase of 85% liquid CO₂ and 15% EtOH with 0.2% TEA using a flowrate of 80 mL/min. The 1^{st} eluting peak was (R)-N-((5-(2-oxa-6-azaspiro[3.3]heptan-6-yl)pyridin-2-yl)methyl)-2-amino-3-methyl-N-(1-(pyrimidin-2-yl)ethyl)quinoline-6-carboxamide (556, 18 mg, > 99% ee) as a brown solid. m/z (ESI): 496.3 (M+H)⁺. ¹H NMR (400 MHz, METHANOL-d₄) δ ppm 8.75 (br s, 2 H), 7.54 - 7.97 (m, 5 H), 7.20 - 7.39 (m, 2 H), 6.89 (br d, J=6.7 Hz, 1 H), 5.37 - 5.71 (m, 1 H), 4.89 - 5.07 (m, 2 H), 4.86 (s, 4 H), 4.08 (s, 4 H), 2.33 (s, 3 H), 1.64 (br d, J=5.6 Hz, 3 H). The 2^{nd} eluting peak was (S)-N-((5-(2-oxa-6-azaspiro[3.3]heptan-6-yl)pyridin-2-yl)methyl)-2-amino-3-methyl-N-(1-(pyrimidin-2-yl)ethyl)quinoline-6-carboxamide (18 mg, > 99% ee) as a brown solid. m/z (ESI): 496.3 (M+H)⁺.

Example 557: (R)-2-amino-N-((5-(3,6-dihydro-2H-pyran-4-yl)pyridin-2-yl)methyl)-3-methyl-N-(1-(pyrimidin-2-yl)ethyl)quinoline-6-carboxamide.

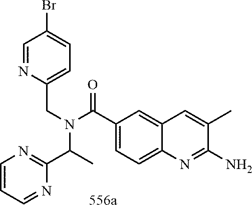

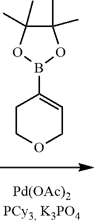

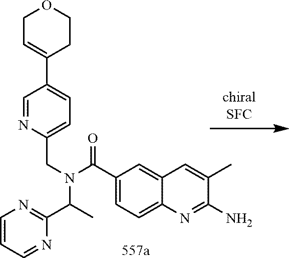

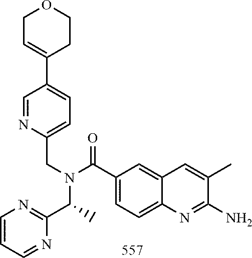

A 50-mL RBF was charged with 2-(3,6-dihydro-2H-pyran-4-yl)-4,4,5,5-tetramethyl-1,3,2-dioxaborolane (726 mg, 3.46 mmol), 556a (550 mg, 1.15 mmol), potassium phosphate tribasic (734 mg, 3.46 mmol), palladium(ii) acetate (39 mg, 0.17 mmol), tricyclohexylphosphine (97 mg, 0.34 mmol), toluene (5.2 mL), and water (0.6 mL). The mixture was purged with N₂ for 10 min then heated at 90° C. for 12 h. The reaction mixture was cooled to RT and concentrated under reduced pressure. The crude residue was purified by silica gel chromatography (0% to 100% (3:1 EtOAc : EtOH) in heptane) to give 2-amino-N-((5-(3,6-dihydro-2H-pyran-4-yl)pyridin-2-yl)methyl)-3-methyl-N-(1-(pyrimidin-2-yl)ethyl)quinoline-6-carboxamide (557a, 135 mg, 0.28 mmol, 24% yield) as a white solid. m/z (ESI): 481.3 (M+H)⁺.

The racemic material was subjected to chiral SFC under the conditions indicated below to give 2 eluents. Chiral SFC: Chiral Technologies OJ column (250 x 21 mm, 5 µm) with a mobile phase of 80% liquid CO₂ and 20% iPrOH with 0.2% TEA using a flowrate of 90 mL/min. The 1^{st} peak was (R)-2-amino-N-((5-(3,6-dihydro-2H-pyran-4-yl)pyridin-2-yl)methyl)-3-methyl-N-(1-(pyrimidin-2-yl)ethyl)quinoline-6-carboxamide (557, 70 mg, > 99% ee) as a white solid. m/z (ESI): 481.3 (M+H)⁺. ¹H NMR (400 MHz, METHANOL-d₄) δ ppm 8.75 (br s, 2 H), 8.49 (d, J=1.9 Hz, 1 H), 7.52 - 8.00 (m, 5 H), 7.41 (br d, J=8.1 Hz, 1 H), 7.33 (br t, J=4.7 Hz, 1 H), 6.29 (dt, J=3.0, 1.4 Hz, 1 H), 5.38 - 5.86 (m, 1 H), 4.91 - 5.13 (m, 1 H), 4.45 - 4.72 (m, 1 H), 4.32 (q, J=2.8 Hz, 2 H), 3.95 (t, J=5.5 Hz, 2 H), 2.49 - 2.57 (m, 2 H), 2.32 (br s, 3 H), 1.69 (br d, J=6.2 Hz, 3 H). The 2^{nd} peak was (S)-2-amino-N-((5-(3,6-dihydro-2H-pyran-4-yl)pyridin-2-yl)methyl)-3-methyl-N-(1-(pyrimidin-2-yl)ethyl)quinoline-6-carboxamide (65 mg, > 98% ee) as a white solid. m/z (ESI): 481.3 (M+H)⁺.

Example 558: 2-amino-N-((5-(5,6-dihydro-2H-pyran-3-yl)-2-pyridinyl)methyl)-3-methyl-N-((1R)-1-(2-pyrimidinyl)ethyl)-6-quinolinecarboxamide.

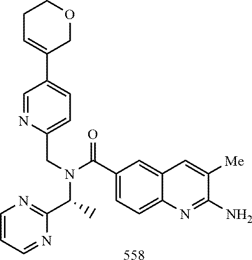

This molecule was prepared in a manner similar to that described for Example 557. SFC conditions: Chiral Technologies AD column (250 x 21 mm, 5 µm) and AD (150 x 21 mm, 5 µm) with a mobile phase of 65% liquid CO₂ and 35% iPrOH with 0.2% TEA using a flowrate of 50 mL/min. The 1^{st} eluting peak was 558 (> 96% ee). m/z (ESI): 481.3 (M+H)⁺. ¹H NMR (400 MHz, METHANOL-d₄) δ ppm 8.75 (br s, 2 H), 8.39 (d, J=1.9 Hz, 1 H), 7.51 - 7.97 (m, 5 H), 7.40 (br d, J=7.9 Hz, 1 H), 7.33 (br t, J=4.6 Hz, 1 H), 6.30 - 6.40 (m, 1 H), 5.36 - 5.91 (m, 1 H), 4.94 - 5.13 (m, 1 H), 4.48 (br d, J=1.7 Hz, 3 H), 3.85 (t, J=5.5 Hz, 2 H), 2.27 - 2.39 (m, 5 H), 1.68 (br d, J=6.1 Hz, 3 H).

Example 559: 2-amino-3-methyl-N-((1R)-1-(2-pyrimidinyl)ethyl)-N-((5-(tetrahydro-2H-pyran-4-yl)-2-pyridinyl)methyl)-6-quinolinecarboxamide.

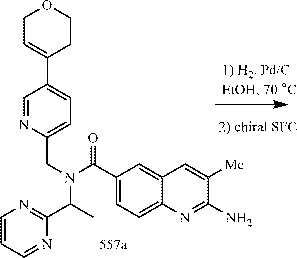

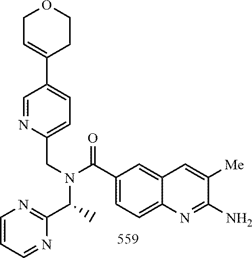

A mixture of 2-amino-N-((5-(3,6-dihydro-2H-pyran-4-yl)pyridin-2-yl)methyl)-3-methyl-N-(1-(pyrimidin-2-yl)ethyl)quinoline-6-carboxamide (557a, 155 mg, 0.32 mmol), EtOH (5.0 mL), and palladium on carbon (34.3 mg, 0.323 mmol) was hydrogenated under 45 psi of hydrogen at 70° C. for 18 h. The reaction mixture was cooled to RT and concentrated under reduced pressure. The crude residue was purified by silica gel chromatography (0% to 100% (3:1 EtOAc : EtOH) in heptane) to give 2-amino-3-methyl-N-(1-(pyrimidin-2-yl)ethyl)-N-((5-(tetrahydro-2H-pyran-4-yl)pyridin-2-yl)methyl)quinoline-6-carboxamide (26.5 mg, 0.055 mmol, 17% yield) as a white solid. m/z (ESI): 483.0 (M+H)⁺. The racemic material was subjected to chiral SFC with 2 steps under the conditions indicated below to give 2 eluents. Chiral SFC: 1) Chiralcel OD-H (2 x 50 cm, 5 µm) with a mobile phase of 60% liquid CO₂ and 40% MeOH with 0.2% DEA using a flowrate of 80 mL/min. 2) Chromega CCC 2 X 25 cm, 5 µm) with a mobile phase of 40% liquid CO₂ and 60% MeOH with 0.2% DEA using a flowrate of 60 mL/min. The 1^{st} peak was 2-amino-3-methyl-N-((1R)-1-(2-pyrimidinyl)ethyl)-N-((5-(tetrahydro-2H-pyran-4-yl)-2-pyridinyl)methyl)-6-quinolinecarboxamide (559, 14 mg, > 99% ee) as a white solid. m/z (ESI): 483.0 (M+H)⁺. ¹H NMR (400 MHz, METHANOL-d₄) δ ppm 8.74 (br s, 2 H), 8.31 (d, J=1.9 Hz, 1 H), 7.53 -7.99 (m, 5 H), 7.29 - 7.45 (m, 2 H), 5.34 - 5.93 (m, 1 H), 4.92 - 5.11 (m, 1 H), 4.00 - 4.13 (m, 2 H), 3.54 -3.65 (m, 2 H), 2.79 - 2.95 (m, 1 H), 2.64 - 2.70 (m, 1 H), 2.32 (br s, 3 H), 1.61 - 1.83 (m, 7 H). The 2^{nd} peak was 2-amino-3-methyl-N-((1S)-1-(2-pyrimidinyl)ethyl)-N-((5-(tetrahydro-2H-pyran-4-yl)-2-pyridinyl)methyl)-6-quinolinecarboxamide (559, 12 mg, > 99% ee) as a white solid. m/z (ESI): 483.0 (M+H)⁺.

Example 560: 2-amino-N-((5-(dimethylcarbamoyl)-2-pyridinyl)methyl)-N-((3-fluoro-2-pyridinyl)methyl)-3-methyl-6-quinolinecarboxamide.

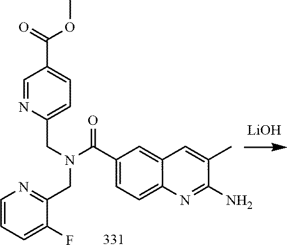

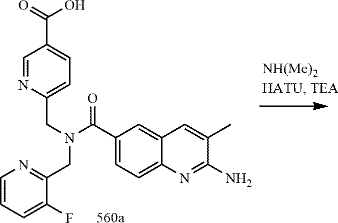

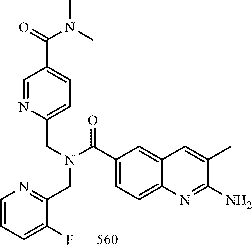

A mixture of methyl 6-((((2-amino-3-methyl-6-quinolinyl)carbonyl)((3-fluoro-2-pyridinyl)methyl)amino)methyl)-3-pyridinecarboxylate (331, 0.103 g, 0.224 mmol), lithium hydroxide hydrate (0.020 g, 0.471 mmol), MeOH (2 mL) and water (1 mL) was stirred at RT for 18 h. The mixture was concentrated in vacuo. 0.5 mL of 1 N HCl was added and a suspension was formed. The mixture was concentrated in vacuo to provide 6-((2-amino-N-((3-fluoropyridin-2-yl)methyl)-3-methylquinoline-6-carboxamido)methyl)nicotinic acid (560a) as a white solid which was used as crude. MS: m/z (ESI): 446 (M+H)⁺.

A mixture of 560a (0.025 g, 0.056 mmol), dimethylamine (0.14 mL of 2.0 M in THF, 0.28 mmol, Aldrich), triethylamine (0.039 mL, 0.281 mmol), and HATU (0.028 g, 0.073 mmol, ChemPep) in DMF (1 mL) was stirred at RT for 18 h. The mixture was purified by reverse phase HPLC (10-70% water in CH₃CN with 0.1% TFA to provide the title compound as a TFA salt (560, 25 mg, 63% yield) as a white solid. MS: m/z (ESI): 473 (M+H)⁺. ¹H NMR (400 MHz, METHANOL-d₄) δ ppm 8.52 (s, 10 H), 4.86 -5.04 (m, 4 H), 3.13 (s, 3 H), 3.03 (s, 3 H), 2.38 (s, 3 H).

Example 561: 2-amino-N-((5-(dimethylcarbamoyl)-2-pyridinyl)methyl)-N-((1R)-1-(3-fluoro-2-pyridinyl)ethyl)-3-methyl-6-quinolinecarboxamide. This molecule was prepared from Example 403 in a manner similar to that described for Example 560. m/z (ESI): 487 (M+H)⁺.

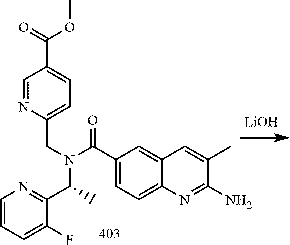

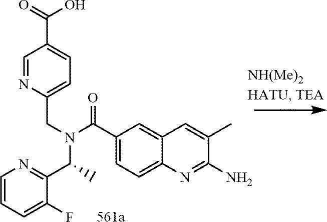

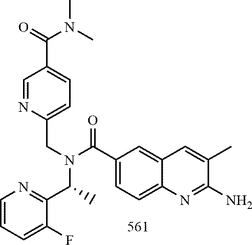

Example 562: 2-amino-N-(cyclobutylmethyl)-3-methyl-N-[[5-(trifluoromethyl)-2-pyridyl]methyl]quinoline-6-carboxamide (Method C-1)

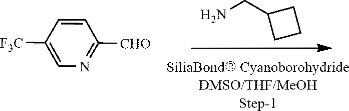

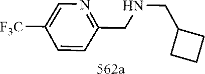

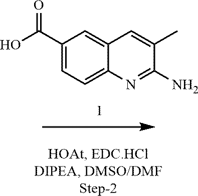

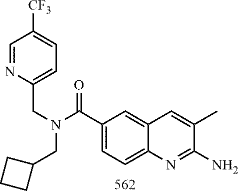

Step1: A solution of cyclobutylmethanamine (1 eq, 100 mM in dry DMSO) and a solution of 5-(trifluoromethyl)pyridine-2-carbaldehyde (1 eq, 100 mM in dry DMSO) were mixed together with equal amounts of dry THF and dry MeOH (25 mM final conc) and MS 3 Å. The mixture was shaken overnight at RT. Thereafter, SiliaBond® Cyanoborohydride (2.5 eq) was added and the reaction mixture was shaken at RT. The reaction mixture was filtered, and the filter-cake was rinsed with CH₃CN. The combined washings and filtrate were concentrated under reduced pressure to give (562a). Step 2: 2-Amino-3-methyl-quinoline-6-carboxylic acid (Int-1, 1 eq, 100 mM in dry DMSO), HOAt (1 eq, 100 mM in dry DMSO) and a solution of EDC and DIPEA (100 mM and 200 mM, respectively in dry DMF) were added in sequence to 562a. The reaction was then shaken at RT overnight and then concentrated under reduced pressure to give crude product that was thereafter purified by HPLC to yield the final product, 2-amino-N-(cyclobutylmethyl)-3-methyl-N-[[5-(trifluoromethyl)-2-pyridyl]methyl]quinoline-6-carboxamide with 99% purity by UV. m/z (ESI): 429.2 (M+H)⁺. R.T.: 2.32 min.

TABLE 13

Examples below were prepared in a manner similar to that described above (Method C-1) for Example 562

| Ex. # | Chemical Structure | Name | LRMS: (ESI+) m/z | R.T. (min) |
|---|---|---|---|---|
| 563 | 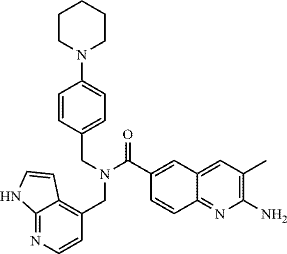 [SMILES: Cc1cc2cc(C(=O)N(Cc3ccc(N4CCCCC4)cc3)Cc3ccnc4[nH]ccc34)ccc2nc1N] | 2-amino-3-methyl-N-[[4-(1-piperidyl)phenyl]methyl]-N-(1H-pyrrolo [2,3-b]pyridin-4-ylmethyl)quinoline-6-carboxamide | 505.2 | 1.56 |
| 564 | 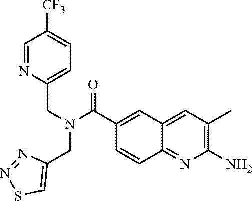 [SMILES: Cc1cc2cc(C(=O)N(Cc3ccc(C(F)(F)F)cn3)Cc3csnn3)ccc2nc1N] | 2-amino-3-methyl-N-(thiadiazol-4-ylmethyl)-N-[[5-(trifluoromethyl)-2-pyridyl]methyl] quinoline-6-carboxamide | 459.1 | 1.84 |
| 565 | 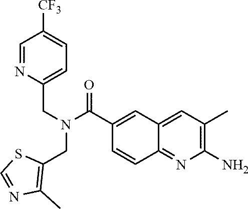 [SMILES: Cc1cc2cc(C(=O)N(Cc3ccc(C(F)(F)F)cn3)Cc3scnc3C)ccc2nc1N] | 2-amino-3-methyl-N-[(4-methylthiazol-5-yl)methyl] -N-[[5-(trifluoromethyl)-2-pyridyl]methyl] quinoline-6-carboxamide | 471.1 | 1.94 |
| 566 | 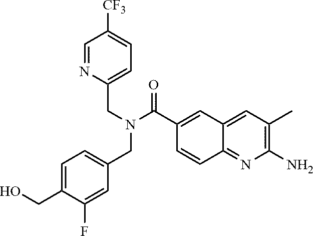 [SMILES: Cc1cc2cc(C(=O)N(Cc3ccc(CO)c(F)c3)Cc3ccc(C(F)(F)F)cn3)ccc2nc1N] | 2-amino-N-[[3-fluoro-4-(hydroxymethyl)phenyl]methyl]-3-methyl-N-[[5-(trifluoromethyl)-2-pyridyl]methyl] quinoline-6-carboxamide | 499.2 | 2.08 |
| 567 | 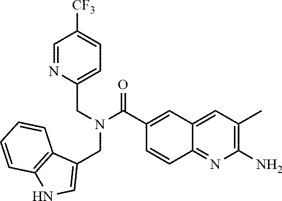 [SMILES: Cc1cc2cc(C(=O)N(Cc3ccc(C(F)(F)F)cn3)Cc3c[nH]c4ccccc34)ccc2nc1N] | 2-amino-N-(1H-indol-3-ylmethyl)-3-methyl-N-[[5-(trifluoromethyl)-2-pyridyl]methyl] quinoline-6-carboxamide | 490.2 | 2.28 |
| 568 | 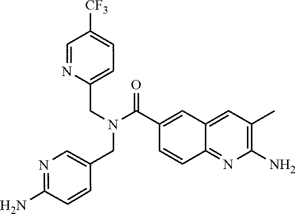 [SMILES: Cc1cc2cc(C(=O)N(Cc3ccc(N)nc3)Cc3ccc(C(F)(F)F)cn3)ccc2nc1N] | 2-amino-N-[(6-amino-3-pyridyl)methyl]-3-methyl-N-[[5-(trifluoromethyl)-2-pyridyl]methyl] quinoline-6-carboxamide | 467.2 | 1.16 |
| 569 | 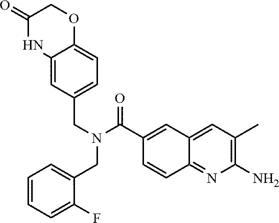 [SMILES: Cc1cc2cc(C(=O)N(Cc3ccc4c(c3)NC(=O)CO4)Cc3ccccc3F)ccc2nc1N] | 2-amino-N-[(2-fluorophenyl)methyl]-3-methyl-N-[(3-oxo-4H-1,4-benzoxazin-6-yl)methyl] quinoline-6-carboxamide | 471.2 | 2.15 |
| 570 | 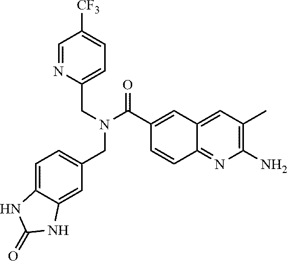 [SMILES: Cc1cc2cc(C(=O)N(Cc3ccc4[nH]c(=O)[nH]c4c3)Cc3ccc(C(F)(F)F)cn3)ccc2nc1N] | 2-amino-3-methyl-N-((2-oxo-2,3-dihydro-1H-benzo[d]imidazol-5-yl)methyl)-N-((5-(trifluoromethyl)pyridin-2-yl)methyl)quinoline-6-carboxamide | 507.2 | 1.87 |
| 571 | 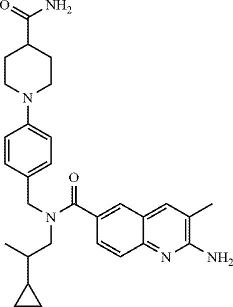 [SMILES: Cc1cc2cc(C(=O)N(Cc3ccc(N4CCC(C(N)=O)CC4)cc3)CC(C)C3CC3)ccc2nc1N] | 2-amino-N-(4-(4-carbamoylpiperidin-1-yl)benzyl)-N-(2-cyclopropylpropyl)-3-methylquinoline-6-carboxamide | 500.3 | 1.95 |
| 572 | 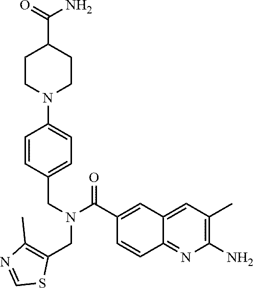 [SMILES: Cc1cc2cc(C(=O)N(Cc3ccc(N4CCC(C(N)=O)CC4)cc3)Cc3scnc3C)ccc2nc1N] | 2-amino-N-(4-(4-carbamoylpiperidin-1 -yl)benzyl)-3-methyl-N-((4-methylthiazol-5-yl)methyl)quinoline-6-carboxamide | 529.2 | 1.40 |
| 573 | 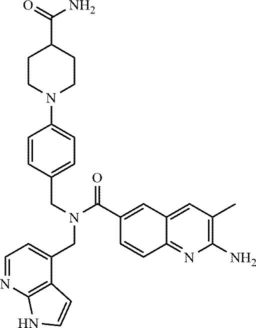 [SMILES: Cc1cc2cc(C(=O)N(Cc3ccc(N4CCC(C(N)=O)CC4)cc3)Cc3ccnc4[nH]ccc34)ccc2nc1N] | N-((1H-pyrrolo[2,3-b]pyridin-4-yl)methyl)-2-amino-N-(4-(4-carbamoylpiperidin-1 -yl)benzyl)-3-methylquinoline-6-carboxamide | 548.3 | 1.33 |
| 574 | 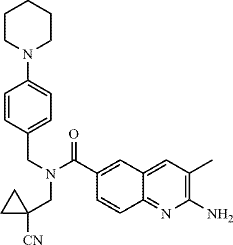 [SMILES: Cc1cc2cc(C(=O)N(Cc3ccc(N4CCCCC4)cc3)CC3(C#N)CC3)ccc2nc1N] | 2-amino-N-((1-cyanocyclopropyl)methyl)-3-methyl-N-(4-(piperidin-1-yl)benzyl)quinoline-6-carboxamide | 454.3 | 1.46 |
| 575 | 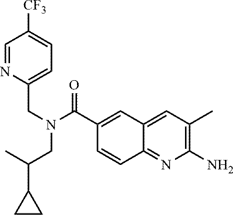 [SMILES: Cc1cc2cc(C(=O)N(Cc3ccc(C(F)(F)F)cn3)CC(C)C3CC3)ccc2nc1N] | 2-amino-N-(2-cyclopropylpropyl)-3-methyl-N-((5-(trifluoromethyl)pyridin-2-yl)methyl)quinoline-6-carboxamide | 443.2 | 2.41 |
| 576 | 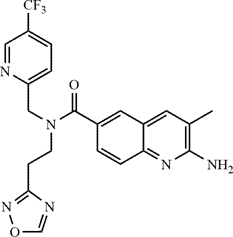 [SMILES: Cc1cc2cc(C(=O)N(CCc3ncon3)Cc3ccc(C(F)(F)F)cn3)ccc2nc1N] | N-(2-(1,2,4-oxadiazol-3-yl)ethyl)-2-amino-3-methyl-N-((5-(trifluoromethyl)pyridin-2-yl)methyl)quinoline-6-carboxamide | 457.2 | 1.76 |
| 577 | 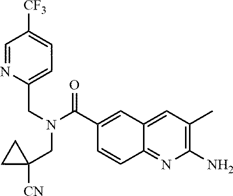 [SMILES: Cc1cc2cc(C(=O)N(Cc3ccc(C(F)(F)F)cn3)CC3(C#N)CC3)ccc2nc1N] | 2-amino-N-((1-cyanocyclopropyl)methyl)-3-methyl-N-((5-(trifluoromethyl)pyridin-2-yl)methyl)quinoline-6-carboxamide | 440.2 | 1.87 |
| 578 | 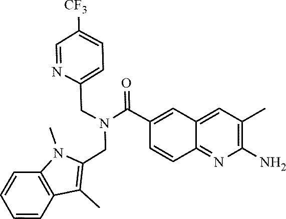 [SMILES: Cc1cc2cc(C(=O)N(Cc3ccc(C(F)(F)F)cn3)Cc3c(C)c4ccccc4n3C)ccc2nc1N] | 2-amino-N-((1,3-dimethyl-1H-indol-2-yl)methyl)-3-methyl-N-((5-(trifluoromethyl)pyridin-2-yl)methyl)quinoline-6-carboxamide | 518.2 | 2.69 |

Example 579: 2-amino-3-methyl-N-[(5-methyloxazol-4-yl)methyl]-N-[[5-(trifluoromethyl)-2-pyridyl]methyl]quinoline-6-carboxamide (Method C-2)

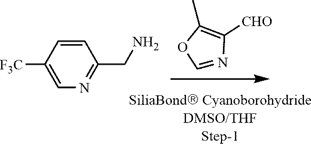

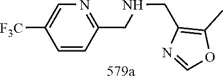

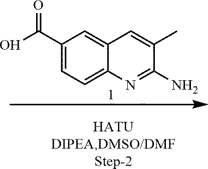

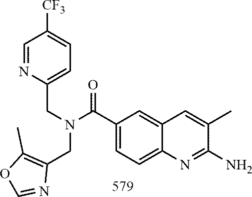

Step 1: Reductive amination was performed as Step-1, Example 562 by using [5-(trifluoromethyl)-2-pyridyl]methanamine and 5-methyloxazole-4-carbaldehyde to form 579a. Step 2: 2-Amino-3-methyl-quinoline-6-carboxylic acid (1 eq, 100 mM in dry DMSO), HATU (1.1 eq. 100 mM in dry DMSO) and DIPEA (10 eq. 1 M in dry DMF) were added in sequence to 579a. The reaction was then shaken at RT overnight, concentrated under reduced pressure, and thereafter purified by HPLC to yield 2-amino-3 -methyl-N-[(5-methyloxazol-4-yl)methyl] -N-[[5 -(trifluoromethyl)-2-pyridyl]methyl]quinoline-6-carboxamide with 99% purity by UV. m/z (ESI): 456.2 (M+H)⁺. R.T.: 2.32 min.

TABLE 14

Examples 580 to 596 were prepared in a manner similar to that described above (Method C-2) for Example 579

| Ex. # | Chemical Structure | Name | LRMS: (ESI + ve ion) m/z | R.T. (min) |
|---|---|---|---|---|
| 580 | 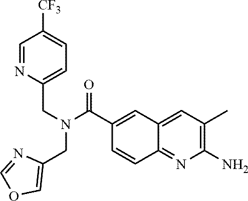 [SMILES: Cc1cc2cc(C(=O)N(Cc3ccc(C(F)(F)F)cn3)Cc3cocn3)ccc2nc1N] | 2-amino-3-methyl-N-(oxazol-4-ylmethyl)-N-[[5-(trifluoromethyl)-2-pyridyl]methyl] quinoline-6-carboxamide | 442.2 | 1.8 |
| 581 | 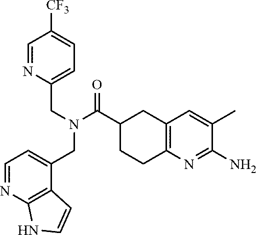 [SMILES: Cc1cc2c(nc1N)CCC(C(=O)N(Cc1ccc(C(F)(F)F)cn1)Cc1ccnc3[nH]ccc13)C2] | N-((1H-pyrrolo[2,3-b]pyridin-4-yl)methyl)-2-amino-3-methyl-N-((5-(trifluoromethyl)pyridin-2-yl)methyl)-5,6,7,8-tetrahydroquinoline-6-carboxamide | 481.1 | 1.79 |
| 582 | 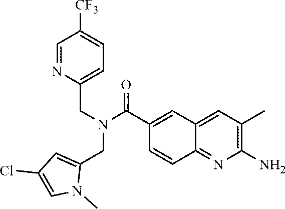 [SMILES: Cc1cc2cc(C(=O)N(Cc3ccc(C(F)(F)F)cn3)Cc3cc(Cl)cn3C)ccc2nc1N] | 2-amino-N-((4-chloro-1-methyl-1H-pyrrol-2-yl)methyl)-3-methyl-N-((5-(trifluoromethyl)pyridin-2-yl)methyl)quinoline-6-carboxamide | 488.2 | 2.34 |
| 583 | 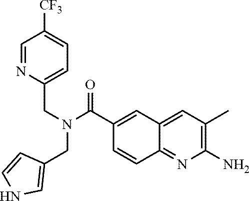 [SMILES: Cc1cc2cc(C(=O)N(Cc3cc[nH]c3)Cc3ccc(C(F)(F)F)cn3)ccc2nc1N] | N-((1H-pyrrol-3-yl)methyl)-2-amino-3-methyl-N-((5-(trifluoromethyl)pyridin-2-yl)methyl)quinoline-6-carboxamide | 440.2 | 1.97 |
| 584 | 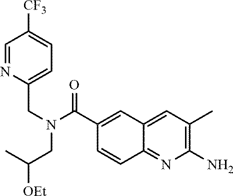 [SMILES: CCOC(C)CN(Cc1ccc(C(F)(F)F)cn1)C(=O)c1ccc2nc(N)c(C)cc2c1] | 2-amino-N-(2-ethoxypropyl)-3-methyl-N-((5-(trifluoromethyl)pyridin-2-yl)methyl)quinoline-6-carboxamide | 447.2 | 2.13 |
| 585 | 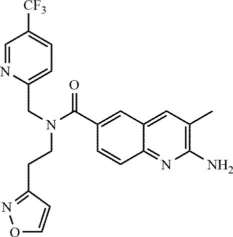 [SMILES: Cc1cc2cc(C(=O)N(CCc3ccon3)Cc3ccc(C(F)(F)F)cn3)ccc2nc1N] | 2-amino-N-(2-(isoxazol-3-yl)ethyl)-3-methyl-N-((5-(trifluoromethyl)pyridin-2-yl)methyl)quinoline-6-carboxamide | 456.2 | 1.83 |
| 586 | 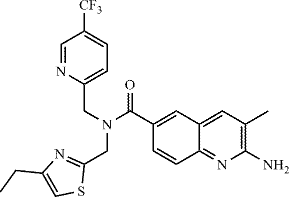 [SMILES: CCc1csc(CN(Cc2ccc(C(F)(F)F)cn2)C(=O)c2ccc3nc(N)c(C)cc3c2)n1] | 2-amino-N-((4-ethylthiazol-2-yl)methyl)-3-methyl-N-((5-(trifluoromethyl)pyridin-2-yl)methyl)quinoline-6-carboxamide | 486.2 | 2.21 |
| 587 | 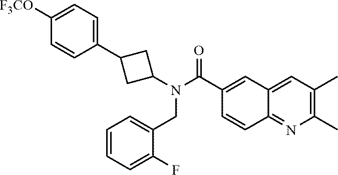 [SMILES: Cc1cc2cc(C(=O)N(Cc3ccccc3F)C3CC(c4ccc(OC(F)(F)F)cc4)C3)ccc2nc1C] | 2-amino-N-(2-fluorobenzyl)-3-methyl-N-(3-(4-(trifluoromethoxy)phenyl)cyclobu tyl)quinoline-6-carboxamide | 524.2 | 2.71 |
| 588 | 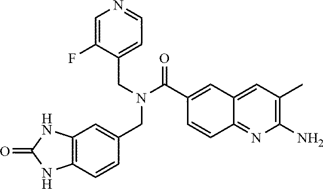 [SMILES: Cc1cc2cc(C(=O)N(Cc3ccc4[nH]c(=O)[nH]c4c3)Cc3ccncc3F)ccc2nc1N] | 2-amino-N-((3-fluoropyridin-4-yl)methyl)-3-methyl-N-((2-oxo-2,3-dihydro-1H-benzo [d] imidazol-5-yl)methyl)quinoline-6-carboxamide | 457.2 | 1.44 |
| 589 | 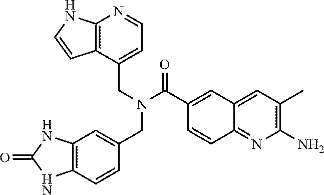 [SMILES: Cc1cc2cc(C(=O)N(Cc3ccc4[nH]c(=O)[nH]c4c3)Cc3ccnc4[nH]ccc34)ccc2nc1N] | N-((1H-pyrrolo[2,3-b]pyridin-4-yl)methyl)-2-amino-3-methyl-N-((2-oxo-2,3-dihydro-1H-benzo [d] imidazol-5-yl)methyl)quinoline-6-carboxamide | 478.2 | 1.31 |
| 590 | 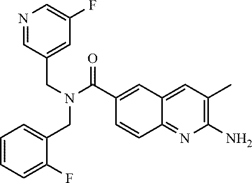 [SMILES: Cc1cc2cc(C(=O)N(Cc3cncc(F)c3)Cc3ccccc3F)ccc2nc1N] | 2-amino-N-(2-fluorobenzyl)-N-((5-fluoropyridin-3-yl)methyl)-3-methylquinoline-6-carboxamide | 419.2 | 1.92 |
| 591 | 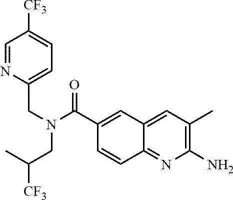 [SMILES: Cc1cc2cc(C(=O)N(Cc3ccc(C(F)(F)F)cn3)CC(C)C(F)(F)F)ccc2nc1N] | 2-amino-3-methyl-N-(3,3,3-trifluoro-2-methylpropyl)-N-((5-(trifluoromethyl)pyridin-2-yl)methyl)quinoline-6-carboxamide | 471.2 | 2.23 |
| 592 | 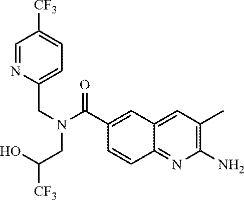 [SMILES: Cc1cc2cc(C(=O)N(Cc3ccc(C(F)(F)F)cn3)CC(O)C(F)(F)F)ccc2nc1N] | 2-amino-3-methyl-N-(3,3,3-trifluoro-2-hydroxypropyl)-N-((5-(trifluoromethyl)pyridin-2-yl)methyl)quinoline-6-carboxamide | 473.1 | 2.04 |
| 593 | 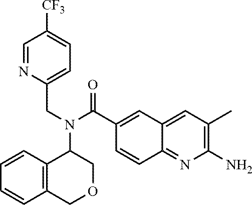 [SMILES: Cc1cc2cc(C(=O)N(Cc3ccc(C(F)(F)F)cn3)C3COCc4ccccc43)ccc2nc1N] | 2-amino-N-(isochroman-4-yl)-3-methyl-N-((5-(trifluoromethyl)pyridin-2-yl)methyl)quinoline-6-carboxamide | 493.2 | 2.22 |
| 594 | 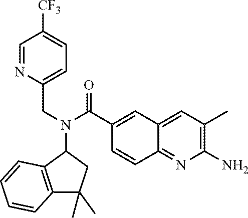 [SMILES: Cc1cc2cc(C(=O)N(Cc3ccc(C(F)(F)F)cn3)C3CC(C)(C)c4ccccc43)ccc2nc1N] | 2-amino-N-(3,3-dimethyl-2,3-dihydro-1H-inden-1-yl)-3-methyl-N-((5-(trifluoromethyl)pyridin-2-yl)methyl)quinoline-6-carboxamide | 505.2 | 2.56 |
| 595 | 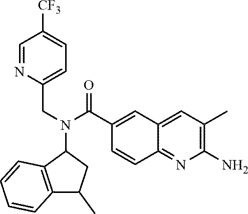 [SMILES: Cc1cc2cc(C(=O)N(Cc3ccc(C(F)(F)F)cn3)C3CC(C)c4ccccc43)ccc2nc1N] | 2-amino-3-methyl-N-(3-methyl-2,3 -dihydro- 1H-inden-1-yl)-N-((5-(trifluoromethyl)pyridin-2-yl)methyl)quinoline-6-carboxamide | 491.2 | 2.50 |
| 596 | 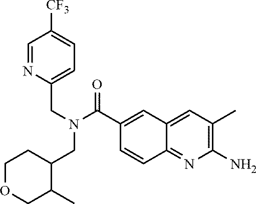 [SMILES: Cc1cc2cc(C(=O)N(Cc3ccc(C(F)(F)F)cn3)CC3CCOCC3C)ccc2nc1N] | 2-amino-3-methyl-N-((3-methyltetrahydro-2H-pyran-4-yl)methyl)-N-((5-(trifluoromethyl)pyridin-2-yl)methyl)quinoline-6-carboxamide | 473.2 | 2.05 |

Example 597: 2-amino-N-((5-fluoropyridin-3-yl)methyl)-3-methyl-N-((5-(trifluoromethyl)pyridin-2-yl)methyl)quinoline-6-carboxamide (Method C-3)

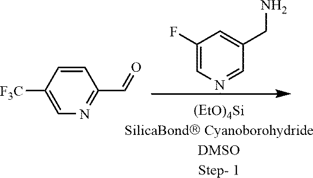

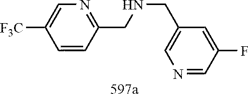

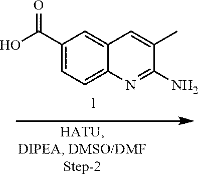

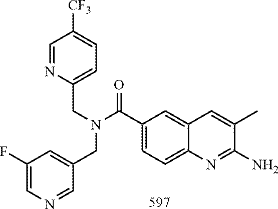

Step 1: To solution of (5-fluoro-3-pyridyl)methanamine (1 eq. 100 mM in dry DMSO) and 5-(trifluoromethyl)pyridine-2-carbaldehyde (1 eq, 100 mM in dry DMSO) was added TEOS (10 eq, neat) and the mixture was shaken at RT overnight. Thereafter, SiliaBond® Cyanoborohydride (2.5 eq) was added and the reaction was again shaken overnight at RT. The reaction mixture was then filtered, and the filter-cake was rinsed with CH₃CN and THF. The combined washings and filtrate were concentrated under reduced pressure to give (597a). Step 2: Performed according to 579 step 2 by using 597a to give 2-amino-N-((5-fluoropyridin-3-yl)methyl)-3-methyl-N-((5-(trifluoromethyl)pyridin-2-yl)methyl)quinoline-6-carboxamide with 99 % purity by UV. m/z (ESI): 470.2 (M+H)⁺. R.T.: 1.99 min.

TABLE 15

Examples 598 to 628 were prepared in a manner similar to that described above (Method C-3) for Example 597

| Ex. # | Chemical Structure | Name | LRMS: (ESI + ve ion) m/z | R.T. (min) |
|---|---|---|---|---|
| 598 | 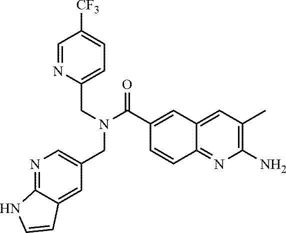 [SMILES: Cc1cc2cc(C(=O)N(Cc3cnc4[nH]ccc4c3)Cc3ccc(C(F)(F)F)cn3)ccc2nc1N] | 2-amino-3-methyl-N-(1H-pyrrolo [2,3 -b]pyridin-5-ylmethyl)-N-[[5-(trifluoromethyl)-2-pyridyl]methyl]quinoline-6-carboxamide | 491.2 | 2.06 |
| 599 | 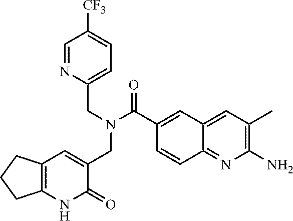 [SMILES: Cc1cc2cc(C(=O)N(Cc3ccc(C(F)(F)F)cn3)Cc3cc4c([nH]c3=O)CCC4)ccc2nc1N] | 2-amino-3-methyl-N-[(2-oxo-1,5,6,7-tetrahydrocyclopenta[b]pyridin-3-yl)methyl]-N-[[5-(trifluoromethyl)-2-pyridyl]methyl] quinoline-6-carboxamide | 508.2 | 2.1 |
| 600 | 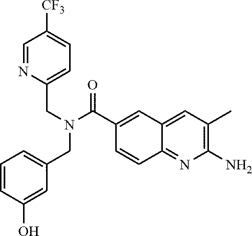 [SMILES: Cc1cc2cc(C(=O)N(Cc3cccc(O)c3)Cc3ccc(C(F)(F)F)cn3)ccc2nc1N] | 2-amino-N-[(3-hydroxyphenyl)methyl]-3-methyl-N-[[5-(trifluoromethyl)-2-pyridyl]methyl] quinoline-6-carboxamide | 467.2 | 2.15 |
| 601 | 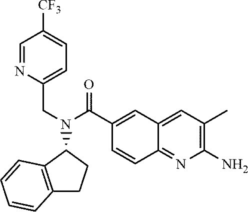 [SMILES: Cc1cc2cc(C(=O)N(Cc3ccc(C(F)(F)F)cn3)[C@@H]3CCc4ccccc43)ccc2nc1N] | 2-amino-N-[(1R)-indan-1-yl]-3-methyl-N-[[5-(trifluoromethyl)-2-pyridyl]methyl] quinoline-6-carboxamide | 477.2 | 2.56 |
| 602 | 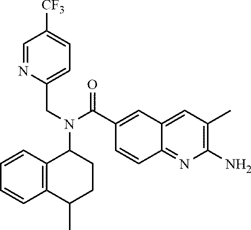 [SMILES: Cc1cc2cc(C(=O)N(Cc3ccc(C(F)(F)F)cn3)C3CCC(C)c4ccccc43)ccc2nc1N] | 2-amino-3-methyl-N-(4-methyl-1,2,3,4-tetrahydronaphthalen-1-yl)-N-((5-(trifluoromethyl)pyridin-2-yl)methyl)quinoline-6-carboxamide | 505.2 | 2.79 |
| 603 | 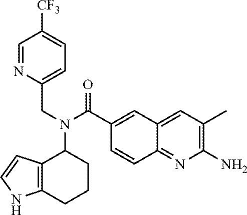 [SMILES: Cc1cc2cc(C(=O)N(Cc3ccc(C(F)(F)F)cn3)C3CCCc4[nH]ccc43)ccc2nc1N] | 2-amino-3-methyl-N-(4,5,6,7-tetrahydro-1H-indol-4-yl)-N-((5-(trifluoromethyl)pyridin-2-yl)methyl)quinoline-6-carboxamide | 480.2 | 2.11 |
| 604 | 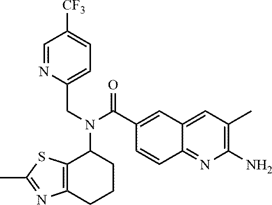 | 2-amino-3-methyl-N-(2-methyl-4,5,6,7-tetrahydrobenzo[d]thiazol-7-yl)-N-((5-(trifluoromethyl)pyridin-2-yl)methyl)quinoline-6-carboxamide | 512.2 | 2.23 |
| 605 | 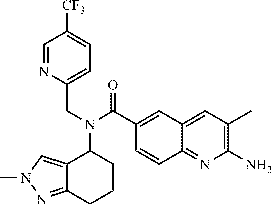 | 2-amino-3-methyl-N-(2-methyl-4,5,6,7-tetrahydro-2H-indazol-4-yl)-N-((5-(trifluoromethyl)pyridin-2-yl)methyl)quinoline-6-carboxamide | 495.2 | 2.11 |
| 606 | 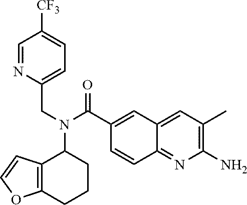 [SMILES: Cc1cc2cc(C(=O)N(Cc3ccc(C(F)(F)F)cn3)C3CCCc4occc43)ccc2nc1N] | 2-amino-3-methyl-N-(4,5,6,7-tetrahydrobenzofuran-4-yl)-N-((5-(trifluoromethyl)pyridin-2-yl)methyl)quinoline-6-carboxamide | 481.2 | 2.45 |
| 607 | 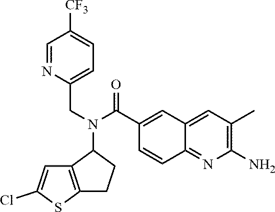 [SMILES: Cc1cc2cc(C(=O)N(Cc3ccc(C(F)(F)F)cn3)C3CCc4sc(Cl)cc43)ccc2nc1N] | 2-amino-N-(2-chloro-5,6-dihydro-4H-cyclopenta[b]thiophen-4-yl)-3-methyl-N-((5-(trifluoromethyl)pyridin-2-yl)methyl)quinoline-6-carboxamide | 517.1 | 2.67 |
| 608 | 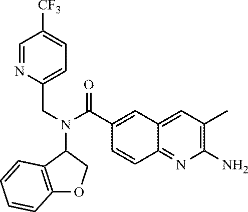 [SMILES: Cc1cc2cc(C(=O)N(Cc3ccc(C(F)(F)F)cn3)C3COc4ccccc43)ccc2nc1N] | 2-amino-N-(2,3-dihydrobenzofuran-3 -yl)-3-methyl-N-((5-(trifluoromethyl)pyridin-2-yl)methyl)quinoline-6-carboxamide | 479.2 | 2.37 |
| 609 | 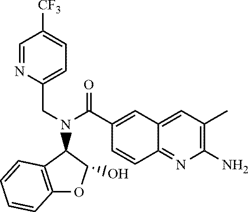 [SMILES: Cc1cc2cc(C(=O)N(Cc3ccc(C(F)(F)F)cn3)[C@@H]3c4ccccc4O[C@H]3O)ccc2nc1N] | 2-amino-N-((1R,2R)-2-hydroxy-2,3-dihydro-1H-inden-1-yl)-3-methyl-N-((5-(trifluoromethyl)pyridin-2-yl)methyl)quinoline-6-carboxamide | 493.2 | 2.38 |
| 610 | 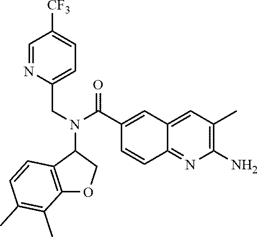 | 2-amino-N-(6,7-dimethyl-2,3-dihydrobenzofuran-3-yl)-3-methyl-N-((5-(trifluoromethyl)pyridin-2-yl)methyl)quinoline-6-carboxamide | 507.2 | 2.67 |
| 611 | 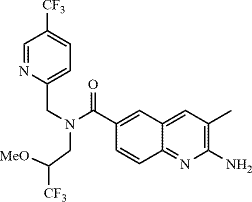 [SMILES: COC(CN(Cc1ccc(C(F)(F)F)cn1)C(=O)c1ccc2nc(N)c(C)cc2c1)C(F)(F)F] | 2-amino-3-methyl-N-(3,3,3-trifluoro-2-methoxypropyl)-N-((5 -(trifluoromethyl)pyridin-2-yl)methyl)quinoline-6-carboxamide | 487.2 | 2.34 |
| 612 | 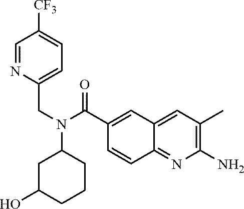 [SMILES: Cc1cc2cc(C(=O)N(Cc3ccc(C(F)(F)F)cn3)C3CCCC(O)C3)ccc2nc1N] | 2-amino-N-(3-hydroxycyclohexyl)-3-methyl-N-((5 -(trifluoromethyl)pyridin-2-yl)methyl)quinoline-6-carboxamide | 459.2 | 1.89 |
| 613 | 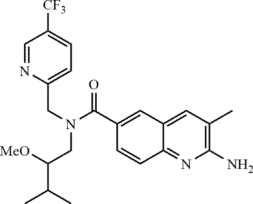 [SMILES: COC(CN(Cc1ccc(C(F)(F)F)cn1)C(=O)c1ccc2nc(N)c(C)cc2c1)C(C)C] | 2-amino-N-(2-methoxy-3-methylbutyl)-3-methyl-N-((5-(trifluoromethyl)pyridin-2-yl)methyl)quinoline-6-carboxamide | 461.2 | 2.37 |
| 614 | 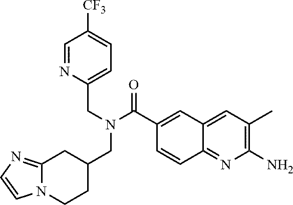 [SMILES: Cc1cc2cc(C(=O)N(Cc3ccc(C(F)(F)F)cn3)CC3CCn4ccnc4C3)ccc2nc1N] | 2-amino-3-methyl-N-((5,6,7,8-tetrahydroimidazo [1,2-a] pyridin-7-yl)methyl)-N-((5-(trifluoromethyl)pyridin-2-yl)methyl)quinoline-6-carboxamide | 495.2 | 1.19 |
| 615 | 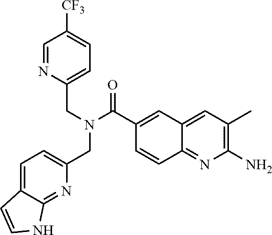 [SMILES: Cc1cc2cc(C(=O)N(Cc3ccc(C(F)(F)F)cn3)Cc3ccc4cc[nH]c4n3)ccc2nc1N] | N-((1H-pyrrolo [2,3-b]pyridin-6-yl)methyl)-2-amino-3-methyl-N-((5-(trifluoromethyl)pyridin-2-yl)methyl)quinoline-6-carboxamide | 491.2 | 2.26 |
| 616 | 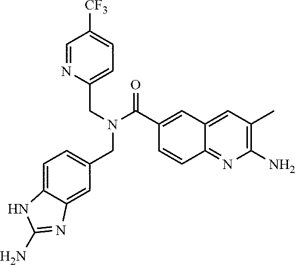 [SMILES: Cc1cc2cc(C(=O)N(Cc3ccc4[nH]c(N)nc4c3)Cc3ccc(C(F)(F)F)cn3)ccc2nc1N] | 2-amino-N-((2-amino-1H-benzo [d]imidazol-5-yl)methyl)-3-methyl-N-((5-(trifluoromethyl)pyridin-2-yl)methyl)quinoline-6-carboxamide | 506.2 | 1.59 |
| 617 | 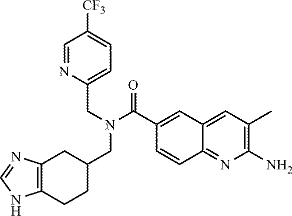 [SMILES: Cc1cc2cc(C(=O)N(Cc3ccc(C(F)(F)F)cn3)CC3CCc4[nH]cnc4C3)ccc2nc1N] | 2-amino-3-methyl-N-((4,5,6,7-tetrahydro-1H-benzo[d]imidazol-5-yl)methyl)-N-((5 -(trifluoromethyl)pyridin-2-yl)methyl)quinoline-6-carboxamide | 495.2 | 1.37 |
| 618 | 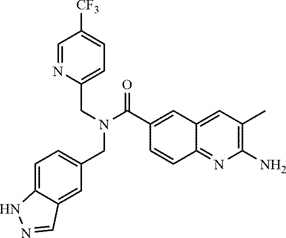 [SMILES: Cc1cc2cc(C(=O)N(Cc3ccc4[nH]ncc4c3)Cc3ccc(C(F)(F)F)cn3)ccc2nc1N] | N-((1H-indazol-5-yl)methyl)-2-amino-3-methyl-N-((5-(trifluoromethyl)pyridin-2-yl)methyl)quinoline-6-carboxamide | 491.2 | 2.07 |
| 619 | 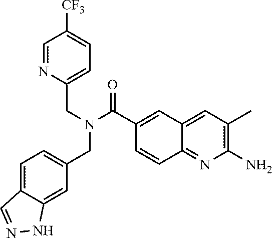 [SMILES: Cc1cc2cc(C(=O)N(Cc3ccc4cn[nH]c4c3)Cc3ccc(C(F)(F)F)cn3)ccc2nc1N] | N-((1H-indazol-6-yl)methyl)-2-amino-3-methyl-N-((5-(trifluoromethyl)pyridin-2-yl)methyl)quinoline-6-carboxamide | 491.2 | 2.17 |
| 620 | 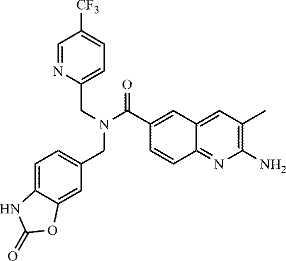 [SMILES: Cc1cc2cc(C(=O)N(Cc3ccc4[nH]c(=O)oc4c3)Cc3ccc(C(F)(F)F)cn3)ccc2nc1N] | 2-amino-3-methyl-N-((2-oxo-2,3 -dihydrobenzo [d]oxazol-6-yl)methyl)-N-((5-(trifluoromethyl)pyridin-2-yl)methyl)quinoline-6-carboxamide | 508.2 | 2.03 |
| 621 | 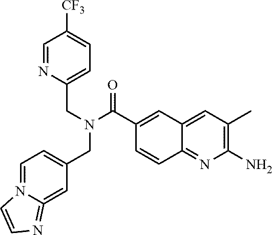 | 2-amino-N-(imidazo[1,2-a]pyridin-7-ylmethyl)-3-methyl-N-((5 -(trifluoromethyl)pyridin-2-yl)methyl)quinoline-6-carboxamide | 491.2 | 1.26 |
| 622 | 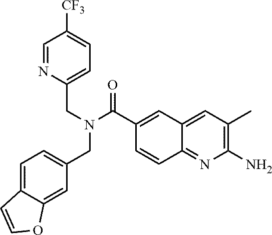 [SMILES: Cc1cc2cc(C(=O)N(Cc3ccc4ccoc4c3)Cc3ccc(C(F)(F)F)cn3)ccc2nc1N] | 2-amino-N-(benzofuran-6-ylmethyl)-3-methyl-N-((5-(trifluoromethyl)pyridin-2-yl)methyl)quinoline-6-carboxamide | 491.2 | 2.49 |
| 623 | 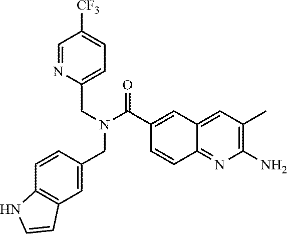 [SMILES: Cc1cc2cc(C(=O)N(Cc3ccc4[nH]ccc4c3)Cc3ccc(C(F)(F)F)cn3)ccc2nc1N] | N-((1H-indol-5-yl)methyl)-2-amino-3-methyl-N-((5-(trifluoromethyl)pyridin-2-yl)methyl)quinoline-6-carboxamide | 490.2 | 2.32 |
| 624 | 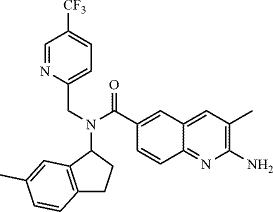 | 2-amino-3-methyl-N-(6-methyl-2,3-dihydro-1H-inden-1-yl)-N-((5-(trifluoromethyl)pyridin-2-yl)methyl)quinoline-6-carboxamide | 491.2 | 2.68 |
| 625 | 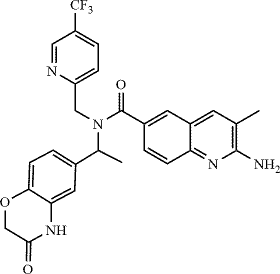 [SMILES: Cc1cc2cc(C(=O)N(Cc3ccc(C(F)(F)F)cn3)C(C)c3ccc4c(c3)NC(=O)CO4)ccc2nc1N] | 2-amino-3-methyl-N-(1-(3-oxo-3,4-dihydro-2H-benzo[b][1,4]oxazin-6-yl)ethyl)-N-((5-(trifluoromethyl)pyridin-2-yl)methyl)quinoline-6-carboxamide | 536.2 | 2.20 |
| 626 | 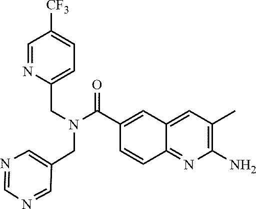 [SMILES: Cc1cc2cc(C(=O)N(Cc3cncnc3)Cc3ccc(C(F)(F)F)cn3)ccc2nc1N] | 2-amino-3-methyl-N-(pyrimidin-5-ylmethyl)-N-((5-(trifluoromethyl)pyridin-2-yl)methyl)quinoline-6-carboxamide | 453.2 | 1.69 |
| 627 | 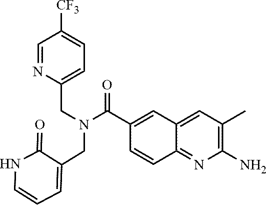 [SMILES: Cc1cc2cc(C(=O)N(Cc3ccc(C(F)(F)F)cn3)Cc3ccc[nH]c3=O)ccc2nc1N] | 2-amino-3-methyl-N-((2-oxo-1,2-dihydropyridin-3-yl)methyl)-N-((5-(trifluoromethyl)pyridin-2-yl)methyl)quinoline-6-carboxamide | 468.2 | 1.73 |
| 628 | 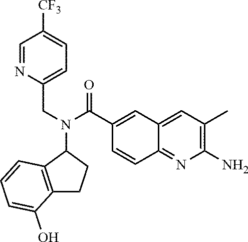 [SMILES: Cc1cc2cc(C(=O)N(Cc3ccc(C(F)(F)F)cn3)C3CCc4c(O)cccc43)ccc2nc1N] | 2-amino-N-(4-hydroxy-2,3-dihydro-1H-inden-1-yl)-3-methyl-N-((5-(trifluoromethyl)pyridin-2-yl)methyl)quinoline-6-carboxamide | 493.2 | 2.12 |

Example 629: 2-amino-N-(2-cyclobutylethyl)-3-methyl-N-((5-(trifluoromethyl)pyridin-2-yl)methyl)quinoline-6-carboxamide (Method C-4)

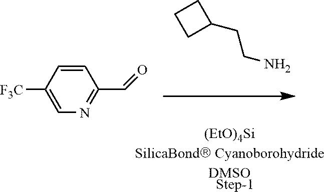

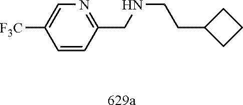

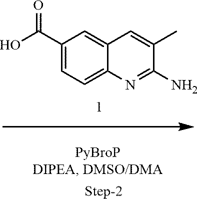

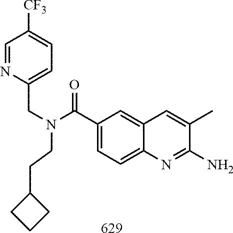

Step1: As example 597 step 1, by using 5-(trifluoromethyl)pyridine-2-carbaldehyde and 2-cyclobutylethanamine to form 629a. Step 2: 2-Amino-3-methyl-quinoline-6-carboxylic acid (1 eq, 100 mM in dry DMSO) and DIPEA (5 eq, 0.5 M in dry DMA) were added in to 629a and the mixture was vigorously mixed. Thereafter, PyBroP (1.1 eq. 110 mM in dry DMA) was added and the reaction was then shaken at RT overnight. Next the reaction was concentrated under reduced pressure and subsequent purification by HPLC gave 2-amino-N-(2-cyclobutylethyl)-3-methyl-N-[[5-(trifluoromethyl)-2-pyridyl]methyl]quinoline-6-carboxamide as the final product with 99% purity by UV. m/z (ESI): 443.2 (M+H)⁺. R.T.: 2.71 min.

TABLE 16

Examples below were prepared in a manner similar to that described above (Method C-4) for Example 629

| Ex. # | Chemical Structure | Name | LRMS: (ESI+) m/z | R.T. (min) |
|---|---|---|---|---|
| 630 | 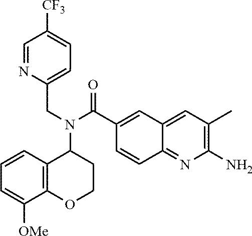 [SMILES: COc1cccc2c1OCCC2N(Cc1ccc(C(F)(F)F)cn1)C(=O)c1ccc2nc(N)c(C)cc2c1] | 2-amino-N-(8-methoxychroman-4-yl)-3-methyl-N-[[5-(trifluoromethyl)-2-pyridyl]methyl] quinoline-6-carboxamide | 523.2 | 2.21 |
| 631 | 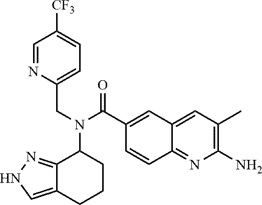 [SMILES: Cc1cc2cc(C(=O)N(Cc3ccc(C(F)(F)F)cn3)C3CCCc4c[nH]nc43)ccc2nc1N] | 2-amino-3-methyl-N-(4,5,6,7-tetrahydro-2H-indazol-7-yl)-N-[[5-(trifluoromethyl)-2-pyridyl]methyl] quinoline-6-carboxamide | 481.2 | 2.24 |
| 632 | 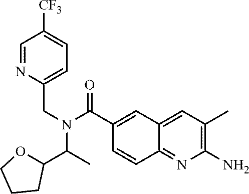 [SMILES: Cc1cc2cc(C(=O)N(Cc3ccc(C(F)(F)F)cn3)C(C)C3CCCO3)ccc2nc1N] | 2-amino-3-methyl-N-(1-tetrahydrofuran-2-ylethyl)-N-[[5-(trifluoromethyl)-2-pyridyl]methyl] quinoline-6-carboxamide | 459.2 | 2.09 |
| 633 | 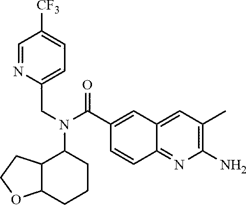 [SMILES: Cc1cc2cc(C(=O)N(Cc3ccc(C(F)(F)F)cn3)C3CCCC4OCCC43)ccc2nc1N] | N-(2,3,3a,4,5,6,7,7a-octahydrobenzofuran-4-yl)-2-amino-3-methyl-N-[[5-(trifluoromethyl)-2-pyridyl]methyl] quinoline-6-carboxamide | 485.2 | 2.09 |
| 634 | 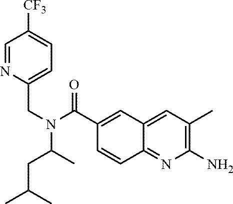 [SMILES: Cc1cc2cc(C(=O)N(Cc3ccc(C(F)(F)F)cn3)C(C)CC(C)C)ccc2nc1N] | 2-amino-N-(1,3-dimethylbutyl)-3-methyl-N-[[5-(trifluoromethyl)-2-pyridyl]methyl] quinoline-6-carboxamide | 445.2 | 2.46 |
| 635 | 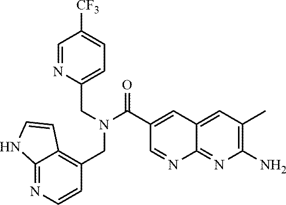 [SMILES: Cc1cc2cc(C(=O)N(Cc3ccc(C(F)(F)F)cn3)Cc3ccnc4[nH]ccc34)cnc2nc1N] | 7-amino-6-methyl-N-(1H-pyrrolo[2,3-b]pyridin-4-ylmethyl)-N-[[5-(trifluoromethyl)-2-pyridyl]methyl]-1,8-naphthyridine-3-carboxamide | 491.2 | 1.94 |
| 636 | 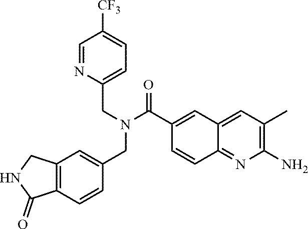 [SMILES: Cc1cc2cc(C(=O)N(Cc3ccc4c(c3)CNC4=O)Cc3ccc(C(F)(F)F)cn3)ccc2nc1N] | 2-amino-3-methyl-N-[(1-oxoisoindolin-5-yl)methyl]-N-[[5-(trifluoromethyl)-2-pyridyl]methyl] quinoline-6-carboxamide | 506.2 | 1.87 |
| 637 | 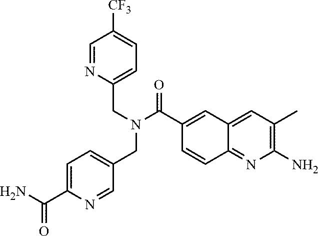 [SMILES: Cc1cc2cc(C(=O)N(Cc3ccc(C(N)=O)nc3)Cc3ccc(C(F)(F)F)cn3)ccc2nc1N] | 2-amino-N-[(6-carbamoyl-3-pyridyl)methyl]-3-methyl-N-[[5-(trifluoromethyl)-2-pyridyl]methyl] quinoline-6-carboxamide | 495.2 | 1.80 |
| 638 | 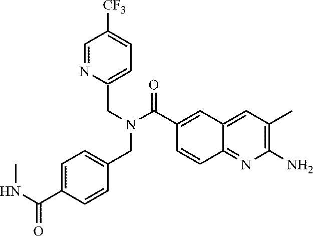 [SMILES: CNC(=O)c1ccc(CN(Cc2ccc(C(F)(F)F)cn2)C(=O)c2ccc3nc(N)c(C)cc3c2)cc1] | 2-amino-3-methyl-N-[[4-(methylcarbamoyl)phenyl]methyl]-N-[[5-(trifluoromethyl)-2-pyridyl]methyl] quinoline-6-carboxamide | 508.2 | 1.95 |
| 639 | 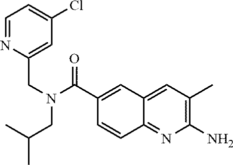 [SMILES: Cc1cc2cc(C(=O)N(Cc3cc(Cl)ccn3)CC(C)C)ccc2nc1N] | 2-amino-N-[(4-chloro-2-pyridyl)methyl]-N-isobutyl-3-methylquinoline-6-carboxamide | 383.2 | 2.07 |
| 640 | 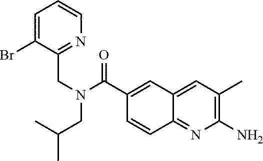 [SMILES: Cc1cc2cc(C(=O)N(Cc3ncccc3Br)CC(C)C)ccc2nc1N] | 2-amino-N-[(3-bromo-2-pyridyl)methyl] -N-isobutyl-3-methylquinoline-6-carboxamide | 427.1 | 2.22 |
| 641 | 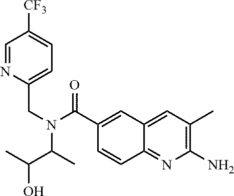 [SMILES: Cc1cc2cc(C(=O)N(Cc3ccc(C(F)(F)F)cn3)C(C)C(C)O)ccc2nc1N] | 2-amino-N-(2-hydroxy-1-methylpropyl)-3-methyl-N-[[5-(trifluoromethyl)-2-pyridyl]methyl] quinoline-6-carboxamide | 433.2 | 1.84 |
| 642 | 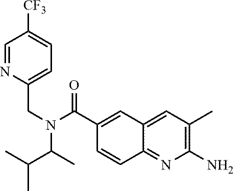 [SMILES: Cc1cc2cc(C(=O)N(Cc3ccc(C(F)(F)F)cn3)C(C)C(C)C)ccc2nc1N] | 2-amino-N-(1,2-dimethylpropyl)-3-methyl-N-[[5-(trifluoromethyl)-2-pyridyl]methyl] quinoline-6-carboxamide | 431.2 | 2.33 |
| 643 | 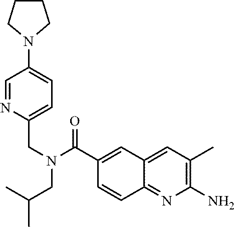 [SMILES: Cc1cc2cc(C(=O)N(Cc3ccc(N4CCCC4)cn3)CC(C)C)ccc2nc1N] | 2-amino-N-isobutyl-3-methyl-N-[(4-pyrrolidin-1-ylphenyl)methyl]quinoline-6-carboxamide | 417.3 | 2.66 |
| 644 | 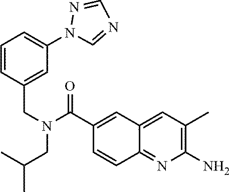 [SMILES: Cc1cc2cc(C(=O)N(Cc3cccc(-n4cncn4)c3)CC(C)C)ccc2nc1N] | 2-amino-N-isobutyl-3-methyl-N-[[3-(1,2,4-triazol-1-yl)phenyl]methyl] quinoline-6-carboxamide | 415.2 | 1.99 |
| 645 | 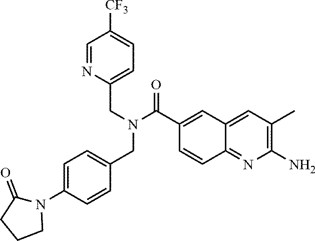 [SMILES: Cc1cc2cc(C(=O)N(Cc3ccc(N4CCCC4=O)cc3)Cc3ccc(C(F)(F)F)cn3)ccc2nc1N] | 2-amino-3-methyl-N-[[4-(2-oxopyrrolidin-1-yl)phenyl]methyl] -N-[[5-(trifluoromethyl)-2-pyridyl]methyl] quinoline-6-carboxamide | 534.2 | 2.21 |
| 646 | 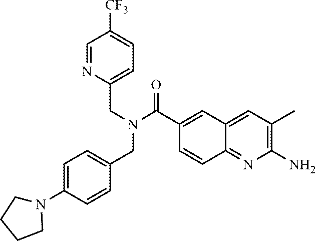 [SMILES: Cc1cc2cc(C(=O)N(Cc3ccc(N4CCCC4)cc3)Cc3ccc(C(F)(F)F)cn3)ccc2nc1N] | 2-amino-3-methyl-N-[(4-pyrrolidin-1-ylphenyl)methyl]-N-[[5-(trifluoromethyl)-2-pyridyl]methyl] quinoline-6-carboxamide | 520.2 | 2.74 |
| 647 | 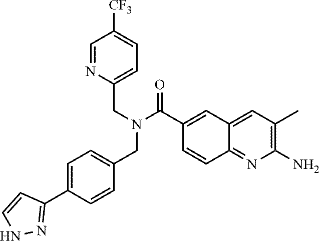 [SMILES: Cc1cc2cc(C(=O)N(Cc3ccc(-c4cc[nH]n4)cc3)Cc3ccc(C(F)(F)F)cn3)ccc2nc1N] | 2-amino-3-methyl-N-[[4-(IH-pyrazol-3-yl)phenyl]methyl]-N-[[5-(trifluoromethyl)-2-pyridyl]methyl] quinoline-6-carboxamide | 517.2 | 2.30 |
| 648 | 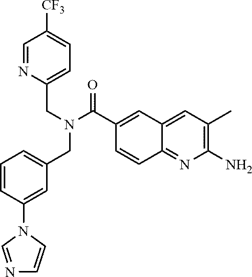 [SMILES: Cc1cc2cc(C(=O)N(Cc3cccc(-n4ccnc4)c3)Cc3ccc(C(F)(F)F)cn3)ccc2nc1N] | 2-amino-N-[(3-imidazol-1-ylphenyl)methyl]-3-methyl-N-[[5-(trifluoromethyl)-2-pyridyl]methyl] quinoline-6-carboxamide | 517.2 | 1.54 |
| 649 | 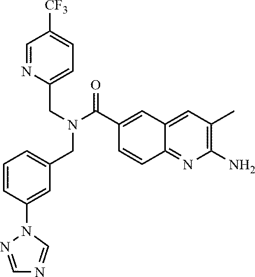 [SMILES: Cc1cc2cc(C(=O)N(Cc3cccc(-n4cncn4)c3)Cc3ccc(C(F)(F)F)cn3)ccc2nc1N] | 2-amino-3-methyl-N-[[3-(1,2,4-triazol-1-yl)phenyl]methyl]-N-[[5-(trifluoromethyl)-2-pyridyl]methyl] quinoline-6-carboxamide | 518.2 | 2.12 |
| 650 | 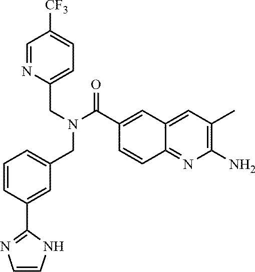 [SMILES: Cc1cc2cc(C(=O)N(Cc3cccc(-c4ncc[nH]4)c3)Cc3ccc(C(F)(F)F)cn3)ccc2nc1N] | 2-amino-N-[ [3 -(1 H-imidazol-2-yl)phenyl]methyl] -3 -methyl-N-[ [5-(trifluoromethyl)-2-pyridyl]methyl] quinoline-6-carboxamide | 517.2 | 1.52 |
| 651 | 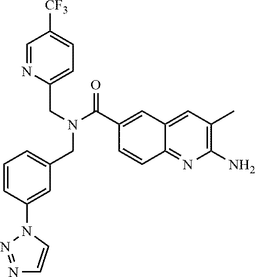 [SMILES: Cc1cc2cc(C(=O)N(Cc3cccc(-n4ccnn4)c3)Cc3ccc(C(F)(F)F)cn3)ccc2nc1N] | 2-amino-3-methyl-N-[[3-(triazol-1-yl)phenyl]methyl] -N-[ [5 -(trifluoromethyl)-2-pyridyl]methyl] quinoline-6-carboxamide | 518.2 | 2.07 |
| 652 | 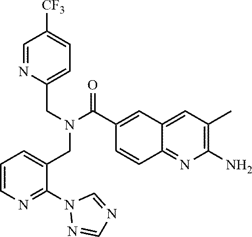 [SMILES: Cc1cc2cc(C(=O)N(Cc3ccc(C(F)(F)F)cn3)Cc3cccnc3-n3cncn3)ccc2nc1N] | 2-amino-3-methyl-N-[[2-(1,2,4-triazol-1-yl)-3-pyridyl] methyl]-N-[[5-(trifluoromethyl)-2-pyridyl]methyl] quinoline-6-carboxamide | 519.2 | 1.91 |
| 653 | 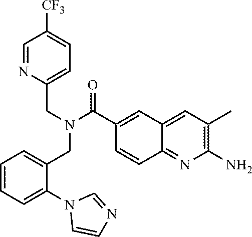 [SMILES: Cc1cc2cc(C(=O)N(Cc3ccc(C(F)(F)F)cn3)Cc3ccccc3-n3ccnc3)ccc2nc1N] | 2-amino-N-[(2-imidazol-1-ylphenyl)methyl]-3-methyl-N-[[5-(trifluoromethyl)-2-pyridyl]methyl] quinoline-6-carboxamide | 517.2 | 1.53 |
| 654 | 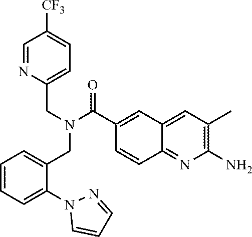 [SMILES: Cc1cc2cc(C(=O)N(Cc3ccc(C(F)(F)F)cn3)Cc3ccccc3-n3cccn3)ccc2nc1N] | 2-amino-3-methyl-N-[(2-pyrazol-1-ylphenyl)methyl]-N-[[5-(trifluoromethyl)-2-pyridyl]methyl] quinoline-6-carboxamide | 517.2 | 2.26 |
| 655 | 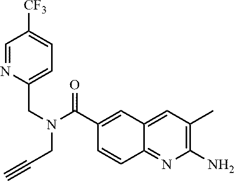 [SMILES: C#CCN(Cc1ccc(C(F)(F)F)cn1)C(=O)c1ccc2nc(N)c(C)cc2c1] | 2-amino-3-methyl-N-prop-2-ynyl-N-[[5-(trifluoromethyl)-2-pyridyl]methyl] quinoline-6-carboxamide | 399.2 | 1.95 |
| 656 | 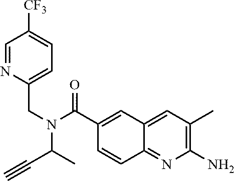 [SMILES: C#CC(C)N(Cc1ccc(C(F)(F)F)cn1)C(=O)c1ccc2nc(N)c(C)cc2c1] | 2-amino-3-methyl-N-(1-methylprop-2-ynyl)-N-[[5-(trifluoromethyl)-2-pyridyl]methyl] quinoline-6-carboxamide | 413.2 | 2.07 |
| 657 | 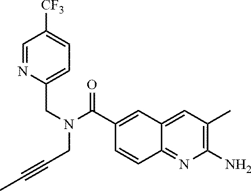 [SMILES: CC#CCN(Cc1ccc(C(F)(F)F)cn1)C(=O)c1ccc2nc(N)c(C)cc2c1] | 2-amino-N-but-2-ynyl-3-methyl-N-[[5-(trifluoromethyl)-2-pyridyl]methyl] quinoline-6-carboxamide | 413.2 | 2.16 |
| 658 | 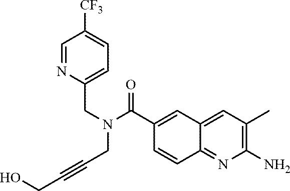 [SMILES: Cc1cc2cc(C(=O)N(CC#CCO)Cc3ccc(C(F)(F)F)cn3)ccc2nc1N] | 2-amino-N-(4-hydroxybut-2-ynyl)-3-methyl-N-[[5-(trifluoromethyl)-2-pyridyl]methyl] quinoline-6-carboxamide | 429.2 | 1.82 |
| 659 | 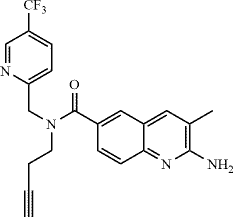 [SMILES: C#CCCN(Cc1ccc(C(F)(F)F)cn1)C(=O)c1ccc2nc(N)c(C)cc2c1] | 2-amino-N-but-3-ynyl-3-methyl-N-[[5-(trifluoromethyl)-2-pyridyl]methyl] quinoline-6-carboxamide | 413.2 | 1.98 |
| 660 | 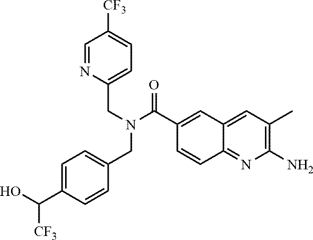 [SMILES: Cc1cc2cc(C(=O)N(Cc3ccc(C(O)C(F)(F)F)cc3)Cc3ccc(C(F)(F)F)cn3)ccc2nc1N] | 2-amino-3-methyl-N-[ [4-(2,2,2-trifluoro-1-hydroxyethyl)phenyl]methyl]-N-[[5-(trifluoromethyl)-2-pyridyl]methyl] quinoline-6-carboxamide | 549.2 | 2.31 |
| 661 | 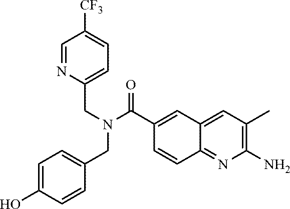 [SMILES: Cc1cc2cc(C(=O)N(Cc3ccc(O)cc3)Cc3ccc(C(F)(F)F)cn3)ccc2nc1N] | 2-amino-N-[(4-hydroxyphenyl)methyl]-3-methyl-N-[[5-(trifluoromethyl)-2-pyridyl]methyl] quinoline-6-carboxamide | 467.2 | 2.07 |
| 662 | 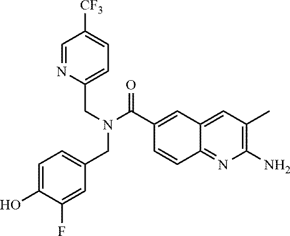 [SMILES: Cc1cc2cc(C(=O)N(Cc3ccc(O)c(F)c3)Cc3ccc(C(F)(F)F)cn3)ccc2nc1N] | 2-amino-N-[(3-fluoro-4-hydroxyphenyl)methyl] -3 -methyl-N-[ [5-(trifluoromethyl)-2-pyridyl]methyl] quinoline-6-carboxamide | 485.2 | 2.10 |
| 663 | 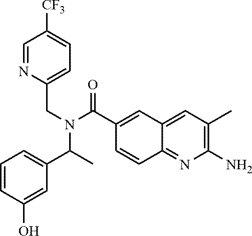 [SMILES: Cc1cc2cc(C(=O)N(Cc3ccc(C(F)(F)F)cn3)C(C)c3cccc(O)c3)ccc2nc1N] | 2-amino-N-[1-(3-hydroxyphenyl)ethyl] -3 -methyl-N-[[5-(trifluoromethyl)-2-pyridyl]methyl] quinoline-6-carboxamide | 481.2 | 2.19 |
| 664 | 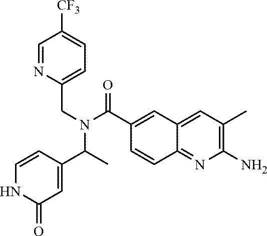 [SMILES: Cc1cc2cc(C(=O)N(Cc3ccc(C(F)(F)F)cn3)C(C)c3cc[nH]c(=O)c3)ccc2nc1N] | 2-amino-3-methyl-N-[1-(2-oxo-1H-pyridin-4-yl)ethyl]-N-[[5-(trifluoromethyl)-2-pyridyl]methyl] quinoline-6-carboxamide | 482.2 | 1.75 |
| 665 | 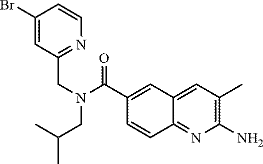 [SMILES: Cc1cc2cc(C(=O)N(Cc3cc(Br)ccn3)CC(C)C)ccc2nc1N] | 2-amino-N-[(4-bromo-2-pyridyl)methyl]-N-isobutyl-3-methylquinoline-6-carboxamide | 427.1 | 2.35 |
| 666 | 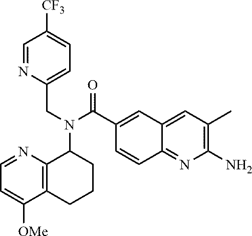 [SMILES: COc1ccnc2c1CCCC2N(Cc1ccc(C(F)(F)F)cn1)C(=O)c1ccc2nc(N)c(C)cc2c1] | 2-amino-N-(4-methoxy-5,6,7,8-tetrahydroquinolin-8-yl)-3-methyl-N-[[5-(trifluoromethyl)-2-pyridyl]methyl] quinoline-6-carboxamide | 522.2 | 2.64 |
| 667 | 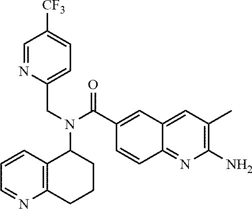 [SMILES: Cc1cc2cc(C(=O)N(Cc3ccc(C(F)(F)F)cn3)C3CCCc4ncccc43)ccc2nc1N] | 2-amino-3-methyl-N-(5,6,7,8-tetrahydroquinolin-5 -yl)-N- [[5 -(trifluoromethyl)-2-pyridyl]methyl] quinoline-6-carboxamide | 492.2 | 1.70 |
| 668 | 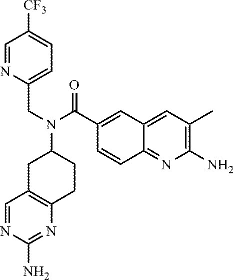 [SMILES: Cc1cc2cc(C(=O)N(Cc3ccc(C(F)(F)F)cn3)C3CCc4nc(N)ncc4C3)ccc2nc1N] | 2-amino-N-(2-amino-5,6,7,8-tetrahydroquinazolin-6-yl)-3-methyl-N-[ [5-(trifluoromethyl)-2-pyridyl]methyl] quinoline-6-carboxamide | 508.2 | 1.80 |
| 669 | 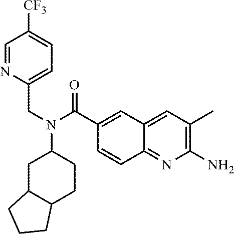 [SMILES: Cc1cc2cc(C(=O)N(Cc3ccc(C(F)(F)F)cn3)C3CCC4CCCC4C3)ccc2nc1N] | N-(2,3,3a,4,5,6,7,7a-octahydro-1H-inden-5-yl)-2-amino-3-methyl-N-[[5-(trifluoromethyl)-2-pyridyl]methyl] quinoline-6-carboxamide | 483.2 | 2.98 |
| 670 | 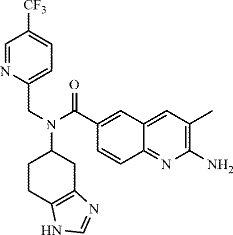 [SMILES: Cc1cc2cc(C(=O)N(Cc3ccc(C(F)(F)F)cn3)C3CCc4[nH]cnc4C3)ccc2nc1N] | 2-amino-3-methyl-N-(4,5,6,7-tetrahydro-1H-benzimidazol-5-yl)-N-[[5-(trifluoromethyl)-2-pyridyl]methyl] quinoline-6-carboxamide | 481.2 | 1.45 |
| 671 | 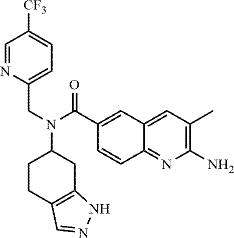 [SMILES: Cc1cc2cc(C(=O)N(Cc3ccc(C(F)(F)F)cn3)C3CCc4cn[nH]c4C3)ccc2nc1N] | 2-amino-3-methyl-N-(4,5,6,7-tetrahydro-1 H-indazol-6-yl)-N-[[5-(trifluoromethyl)-2-pyridyl]methyl] quinoline-6-carboxamide | 481.2 | 2.18 |
| 672 | 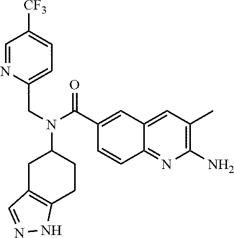 [SMILES: Cc1cc2cc(C(=O)N(Cc3ccc(C(F)(F)F)cn3)C3CCc4[nH]ncc4C3)ccc2nc1N] | 2-amino-3-methyl-N-(4,5,6,7-tetrahydro-1H-mdazol-5-yl)-N-[[5-(trifluoromethyl)-2-pyridyl]methyl] quinoline-6-carboxamide | 481.2 | 2.06 |
| 673 | 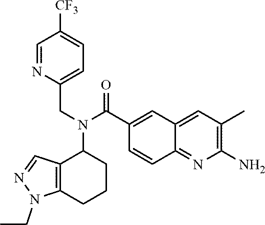 [SMILES: CCn1ncc2c1CCCC2N(Cc1ccc(C(F)(F)F)cn1)C(=O)c1ccc2nc(N)c(C)cc2c1] | 2-amino-N-(1 -ethy1-4,5,6,7-tetrahydroindazol-4-yl)-3-methyl-N-[[5-(trifluoromethyl)-2-pyridyl]methyl] quinoline-6-carboxamide | 509.2 | 2.37 |
| 674 | 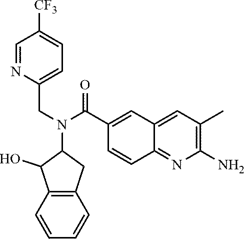 [SMILES: Cc1cc2cc(C(=O)N(Cc3ccc(C(F)(F)F)cn3)C3Cc4ccccc4C3O)ccc2nc1N] | 2-amino-N-(1-hydroxyindan-2-yl)-3-methyl-N-[[5-(trifluoromethyl)-2-pyridyl]methyl] quinoline-6-carboxamide | 493.2 | 2.51 |
| 675 | 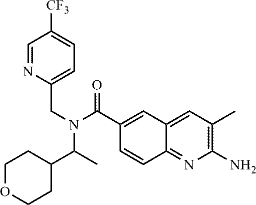 [SMILES: Cc1cc2cc(C(=O)N(Cc3ccc(C(F)(F)F)cn3)C(C)C3CCOCC3)ccc2nc1N] | 2-amino-3-methyl-N-(1-tetrahydropyran-4-ylethyl)-N-[[5-(trifluoromethyl)-2-pyridyl]methyl] quinoline-6-carboxamide | 473.2 | 2.28 |
| 676 | 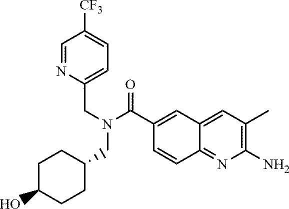 [SMILES: Cc1cc2cc(C(=O)N(Cc3ccc(C(F)(F)F)cn3)C[C@H]3CC[C@H](O)CC3)ccc2nc1N] | 2-amino-N-[(4-hydroxycyclohexyl)methyl]-3-methyl-N-[[5-(trifluoromethyl)-2-pyridyl]methyl] quinoline-6-carboxamide | 473.2 | 2.16 |
| 677 | 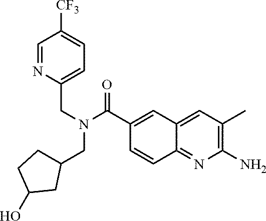 [SMILES: Cc1cc2cc(C(=O)N(Cc3ccc(C(F)(F)F)cn3)CC3CCC(O)C3)ccc2nc1N] | 2-amino-N-[(3-hydroxycyclopentyl)methyl]-3-methyl-N-[[5-(trifluoromethyl)-2-pyridyl]methyl] quinoline-6-carboxamide | 459.2 | 2.12 |
| 678 | 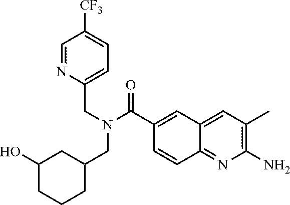 [SMILES: Cc1cc2cc(C(=O)N(Cc3ccc(C(F)(F)F)cn3)CC3CCCC(O)C3)ccc2nc1N] | 2-amino-N-[(3-hydroxycyclohexyl)methyl]-3-methyl-N-[[5-(trifluoromethyl)-2-pyridyl]methyl] quinoline-6-carboxamide | 473.2 | 2.28 |
| 679 | 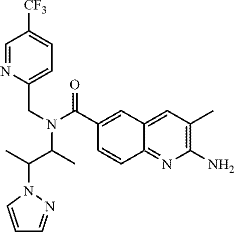 [SMILES: Cc1cc2cc(C(=O)N(Cc3ccc(C(F)(F)F)cn3)C(C)C(C)n3cccn3)ccc2nc1N] | 2-amino-3-methyl-N-(1 -methyl-2-pyrazol-1-yl-propyl)-N-[[5-(trifluoromethyl)-2-pyridyl]methyl] quinoline-6-carboxamide | 483.2 | 2.29 |
| 680 | 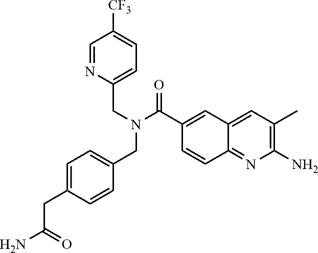 [SMILES: Cc1cc2cc(C(=O)N(Cc3ccc(CC(N)=O)cc3)Cc3ccc(C(F)(F)F)cn3)ccc2nc1N] | 2-amino-N-[ [4-(2-amino-2-oxoethyl)phenyl]methyl]-3-methyl-N-[[5-(trifluoromethyl)-2-pyridyl]methyl] quinoline-6-carboxamide | 508.2 | 2.11 |
| 681 | 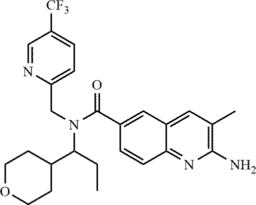 [SMILES: CCC(C1CCOCC1)N(Cc1ccc(C(F)(F)F)cn1)C(=O)c1ccc2nc(N)c(C)cc2c1] | 2-amino-3-methyl-N-(1-tetrahydropyran-4-ylpropyl)-N-[[5-(trifluoromethyl)-2-pyridyl]methyl] quinoline-6-carboxamide | 487.2 | 2.48 |
| 682 | 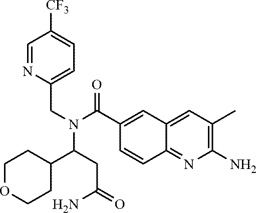 [SMILES: Cc1cc2cc(C(=O)N(Cc3ccc(C(F)(F)F)cn3)C(CC(N)=O)C3CCOCC3)ccc2nc1N] | 2-amino-N-(3 -amino-3 -oxo-1 -tetrahydropyran-4-yl-propyl)-3-methyl-N-[[5-(trifluoromethyl)-2-pyridyl]methyl] quinoline-6-carboxamide | 516.2 | 1.93 |
| 683 | 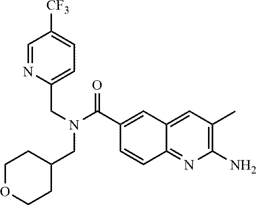 [SMILES: Cc1cc2cc(C(=O)N(Cc3ccc(C(F)(F)F)cn3)CC3CCOCC3)ccc2nc1N] | 2-amino-3-methyl-N-(tetrahydropyran-4-ylmethyl)-N-[[5-(trifluoromethyl)-2-pyridyl]methyl] quinoline-6-carboxamide | 459.2 | 2.16 |
| 684 | 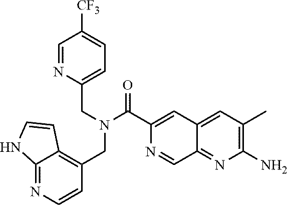 [SMILES: Cc1cc2cc(C(=O)N(Cc3ccc(C(F)(F)F)cn3)Cc3ccnc4[nH]ccc34)ncc2nc1N] | 2-amino-3-methyl-N-(1H-pyrrolo[2,3-b]pyridin-4-ylmethyl)-N-[[5-(trifluoromethyl)-2-pyridyl]methyl]-1,7-naphthyridine-6-carboxamide | 492.2 | 2.17 |
| 685 | 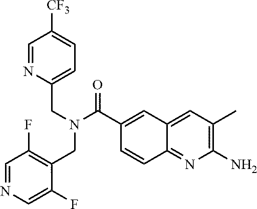 [SMILES: Cc1cc2cc(C(=O)N(Cc3ccc(C(F)(F)F)cn3)Cc3c(F)cncc3F)ccc2nc1N] | 2-amino-N-[(3,5-difluoro-4-pyridyl)methyl]-3-methyl-N-[[4-(trifluoromethyl)phenyl]methyl] quino line-6-carboxamide | 487.2 | 2.46 |
| 686 | 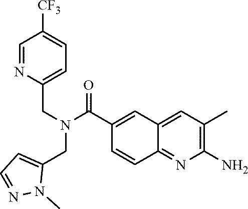 [SMILES: Cc1cc2cc(C(=O)N(Cc3ccc(C(F)(F)F)cn3)Cc3ccnn3C)ccc2nc1N] | 2-amino-3-methyl-N-[(2-methylpyrazol-3-yl)methyl]-N-[[5-(trifluoromethyl)-2-pyridyl]methyl] quinoline-6-carboxamide | 455.2 | 1.99 |
| 687 | 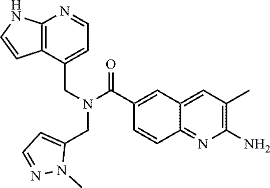 [SMILES: Cc1cc2cc(C(=O)N(Cc3ccnc4[nH]ccc34)Cc3ccnn3C)ccc2nc1N] | 2-amino-3-methyl-N-[(2-methylpyrazol-3 -yl)methy1] -N-(1 H-pyrrolo [2,3 -b]pyridin-4-ylmethyl)quinoline-6-carboxamide | 426.2 | 1.43 |
| 688 | 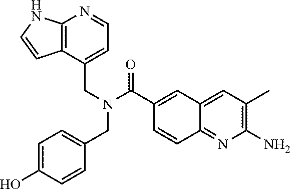 [SMILES: Cc1cc2cc(C(=O)N(Cc3ccc(O)cc3)Cc3ccnc4[nH]ccc34)ccc2nc1N] | 2-amino-N-[(4-hydroxyphenyl)methyl]-3-methyl-N-(1H-pyrrolo[2,3-b]pyridin-4-ylmethyl)quinoline-6-carboxamide | 438.2 | 1.53 |
| 689 | 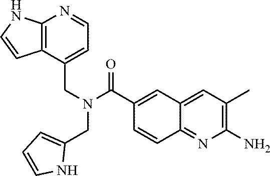 [SMILES: Cc1cc2cc(C(=O)N(Cc3ccc[nH]3)Cc3ccnc4[nH]ccc34)ccc2nc1N] | 2-amino-3-methyl-N-(1H-pyrrolo[2,3-b]pyridin-4-ylmethyl)-N-(1H-pyrrol-2-ylmethyl)quinoline-6-carboxamide | 411.2 | 1.67 |
| 690 | 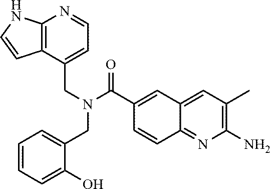 [SMILES: Cc1cc2cc(C(=O)N(Cc3ccccc3O)Cc3ccnc4[nH]ccc34)ccc2nc1N] | 2-amino-N-[(2-hydroxyphenyl)methyl]-3-methyl-N-(1H-pyrrolo[2,3-b]pyridin-4-ylmethyl)quinoline-6-carboxamide | 438.2 | 1.80 |
| 691 | 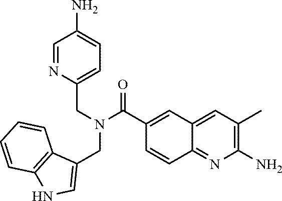 [SMILES: Cc1cc2cc(C(=O)N(Cc3ccc(N)cn3)Cc3c[nH]c4ccccc34)ccc2nc1N] | 2-amino-N-[(5-amino-2-pyridyl)methyl]-N-(1H-indol-3-ylmethyl)-3-methyl-quinoline-6-carboxamide | 437.2 | 1.34 |
| 692 | 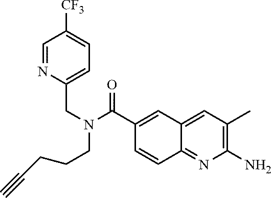 [SMILES: C#CCCCN(Cc1ccc(C(F)(F)F)cn1)C(=O)c1ccc2nc(N)c(C)cc2c1] | 2-amino-3-methyl-N-pent-4-ynyl-N-[[5-(trifluoromethyl)-2-pyridyl]methyl] quinoline-6-carboxamide | 427.2 | 2.27 |
| 693 | 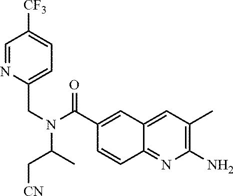 [SMILES: Cc1cc2cc(C(=O)N(Cc3ccc(C(F)(F)F)cn3)C(C)CC#N)ccc2nc1N] | 2-amino-N-(2-cyano-1-methyl-ethyl)-3-methyl-N-[[5-(trifluoromethyl)-2-pyridyl]methyl] quinoline-6-carboxamide | 428.2 | 1.96 |
| 694 | 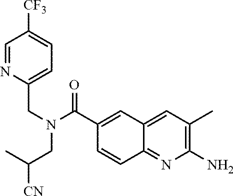 [SMILES: Cc1cc2cc(C(=O)N(Cc3ccc(C(F)(F)F)cn3)CC(C)C#N)ccc2nc1N] | 2-amino-N-(2-cyanopropyl)-3-methyl-N-[[5-(trifluoromethyl)-2-pyridyl]methyl] quinoline-6-carboxamide | 428.2 | 1.95 |
| 695 | 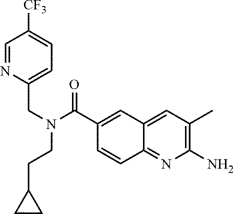 [SMILES: Cc1cc2cc(C(=O)N(CCC3CC3)Cc3ccc(C(F)(F)F)cn3)ccc2nc1N] | 2-amino-N-(2-cyclopropylethyl)-3-methyl-N-[[5-(trifluoromethyl)-2-pyridyl]methyl] quinoline-6-carboxamide | 429.2 | 2.50 |
| 696 | 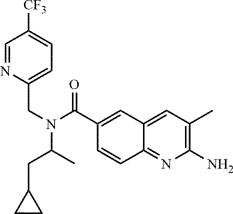 [SMILES: Cc1cc2cc(C(=O)N(Cc3ccc(C(F)(F)F)cn3)C(C)CC3CC3)ccc2nc1N] | 2-amino-N-(2-cyclopropyl-1-methylethyl)-3-methyl-N-[[5-(trifluoromethyl)-2-pyridyl]methyl] quinoline-6-carboxamide | 443.2 | 2.56 |
| 697 | 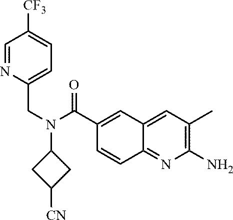 [SMILES: Cc1cc2cc(C(=O)N(Cc3ccc(C(F)(F)F)cn3)C3CC(C#N)C3)ccc2nc1N] | 2-amino-N-(3-cyanocyclobutyl)-3-methyl-N-[[5-(trifluoromethyl)-2-pyridyl]methyl] quinoline-6-carboxamide | 440.2 | 1.90 |
| 698 | 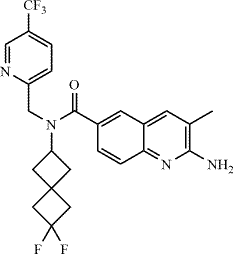 [SMILES: Cc1cc2cc(C(=O)N(Cc3ccc(C(F)(F)F)cn3)C3CC4(C3)CC(F)(F)C4)ccc2nc1N] | 2-amino-N-(2,2-difluorospiro [3.3]heptan-6-yl)-3-methyl-N-[[5-(trifluoromethyl)-2-pyridyl]methyl] quinoline-6-carboxamide | 491.2 | 2.55 |
| 699 | 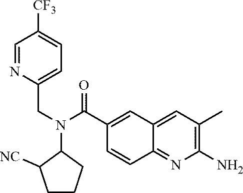 | 2-amino-N-(2-cyanocyclopentyl)-3-methyl-N-[[5-(trifluoromethyl)-2-pyridyl]methyl] quinoline-6-carboxamide | 454.2 | 2.15 |
| 700 | 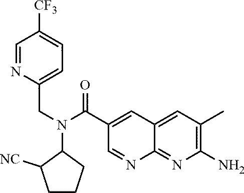 | 7-amino-N-(2-cyanocyclopentyl)-6-methyl-N-[[5-(trifluoromethyl)-2-pyridyl]methyl]-1,8-naphthyridine-3-carboxamide | 455.2 | 2.10 |
| 701 | 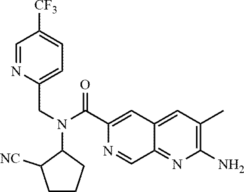 | 2-amino-N-(2-cyanocyclopentyl)-3-methyl-N-[[5-(trifluoromethyl)-2-pyridyl]methyl]-1,7-naphthyridine-6-carboxamide | 455.2 | 2.26 |
| 702 | 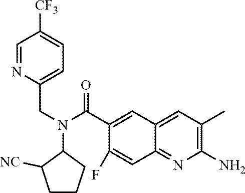 | 2-amino-N-(2-cyanocyclopentyl)-7-fluoro-3-methyl-N-[[5-(trifluoromethyl)-2-pyridyl]methyl] quinoline-6-carboxamide | 472.2 | 2.14 |
| 703 | 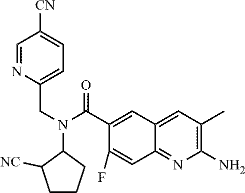 | 2-amino-N-(2-cyanocyclopentyl)-N-[(5-cyano-2-pyridyl)methyl] -7-fluoro-3-methyl-quinoline-6-carboxamide | 429.2 | 1.64 |
| 704 | 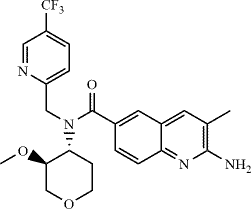 | 2-amino-N-[(3 S,4R)-3-methoxytetrahydropyran-4-yl]-3-methyl-N-[[5-(trifluoromethyl)-2-pyridyl]methyl] quinoline-6-carboxamide | 475.2 | 2.08 |
| 705 | 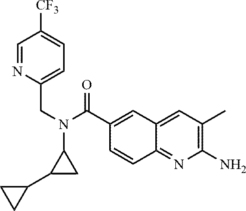 [SMILES: Cc1cc2cc(C(=O)N(Cc3ccc(C(F)(F)F)cn3)C3CC3C3CC3)ccc2nc1N] | 2-amino-N-(2-cyclopropylcyclopropyl)-3-methyl-N-[[5 -(trifluoromethyl)-2-pyridyl]methyl] quinoline-6-carboxamide | 441.2 | 2.51 |
| 706 | 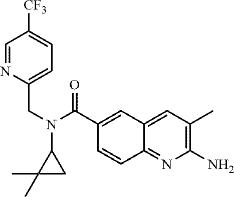 [SMILES: Cc1cc2cc(C(=O)N(Cc3ccc(C(F)(F)F)cn3)C3CC3(C)C)ccc2nc1N] | 2-amino-N-(2,2-dimethylcyclopropyl)-3-methyl-N-[[5-(trifluoromethyl)-2-pyridyl]methyl] quinoline-6-carboxamide | 429.2 | 2.44 |
| 827 | 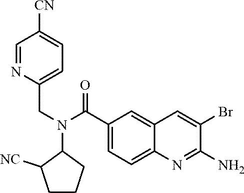 | 2-amino-3-bromo-N-(2-cyanocyclopentyl)-N-((5-cyanopyridin-2-yl)methyl)quinoline-6-carboxamide | 477.1 | 2.05 |
| 828 | 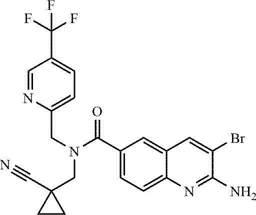 [SMILES: N#CC1(CN(Cc2ccc(C(F)(F)F)cn2)C(=O)c2ccc3nc(N)c(Br)cc3c2)CC1] | 2-amino-3-bromo-N-((1-cyanocyclopropyl)methyl)-N-((5-(trifluoromethyl)pyridin-2-yl)methyl)quinoline-6-carboxamide | 506.1 | 2.50 |
| 829 | 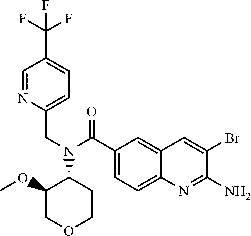 [SMILES: CO[C@@H]1COCC[C@H]1N(Cc1ccc(C(F)(F)F)cn1)C(=O)c1ccc2nc(N)c(Br)cc2c1] | 2-amino-3-bromo-N-((3S,4R)-3-methoxytetrahydro-2H-pyran-4-yl)-N-((5-(trifluoromethyl)pyridin-2-yl)methyl) quinoline-6-carboxamide | 541.1 | 2.57 |
| 830 | 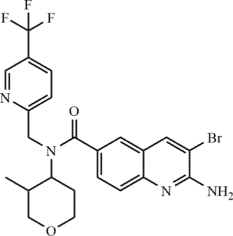 [SMILES: CC1COCCC1N(Cc1ccc(C(F)(F)F)cn1)C(=O)c1ccc2nc(N)c(Br)cc2c1] | 2-amino-3-bromo-N-(3-methyltetrahydro-2H-pyran-4-yl)-N-((5 -(trifluoromethyl)pyridin-2-yl)methyl)quinoline-6-carboxamide | 525.1 | 2.66 |
| 831 | 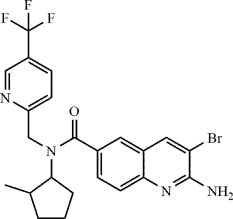 [SMILES: CC1CCCC1N(Cc1ccc(C(F)(F)F)cn1)C(=O)c1ccc2nc(N)c(Br)cc2c1] | 2-amino-3-bromo-N-(2-methylcyclopentyl)-N-((5-(trifluoromethyl)pyridin-2-yl)methyl)quinoline-6-carboxamide | 509.1 | 3.18 |
| 832 | 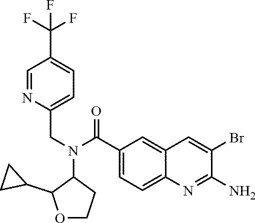 [SMILES: Nc1nc2ccc(C(=O)N(Cc3ccc(C(F)(F)F)cn3)C3CCOC3C3CC3)cc2cc1Br] | 2-amino-3-bromo-N-(2-cyclopropyltetrahydrofuran-3-yl)-N-((5 -(trifluoromethyl)pyridin-2-yl)methyl)quinoline-6-carboxamide | 537.1 | 2.76 |

Example 708: 2-amino-3-methyl-N-[(4-methylthiazol-2-yl)methyl]-N-[[5-(trifluoromethyl)-2-pyridyl]methyl]quinoline-6-carboxamide (Method D)

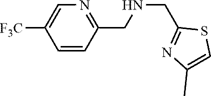 [SMILES: Cc1csc(CNCc2ccc(C(F)(F)F)cn2)n1]

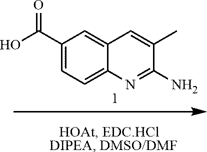

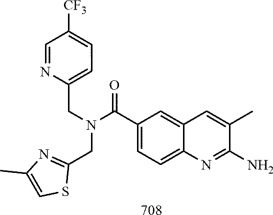

Performed as method C-2, Step 2 by using N-[(4-methylthiazol-2-yl)methyl]-1-[5-(trifluoromethyl)-2-pyridyl]methanamine (100 mM in dry DMSO) and 2-amino-3-methyl-quinoline-6-carboxylic acid (100 mM in dry DMSO) to yield 2-amino-3-methyl-N-[(4-methylthiazol-2-yl)methyl]-N-[[5-(trifluoromethyl)-2-pyridyl]methyl]quinoline-6-carboxamide with a 99% purity by UV. m/z (ESI): 472.1 (M+H)⁺. R.T.: 2.12 min.

TABLE 17

Examples 709 to 711 were prepared in a manner similar to that described above (Method D) for Example 708

| Ex. # | Chemical Structure | Name | LRMS: (ESI + ve ion) m/z | R.T. (min) |
|---|---|---|---|---|
| 709 | 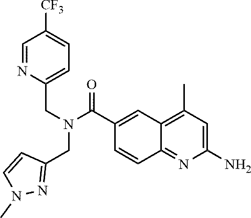 [SMILES: Cc1cc(N)nc2ccc(C(=O)N(Cc3ccc(C(F)(F)F)cn3)Cc3ccn(C)n3)cc12] | 2-amino-4-methyl-N-[(1-methylpyrazol-3-yl)methyl]-N-[[5 -(trifluoromethyl)-2-pyridyl] methyl] quinoline-6-carboxamide | 455.2 | 1.94 |
| 710 | 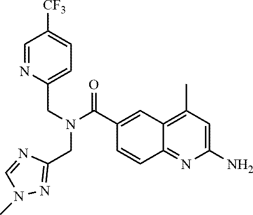 [SMILES: Cc1cc(N)nc2ccc(C(=O)N(Cc3ccc(C(F)(F)F)cn3)Cc3ncn(C)n3)cc12] | 2-amino-4-methyl-N-[(1-methyl-1,2,4-triazol-3-yl)methyl]-N-[[5-(trifluoromethyl)-2-pyridyl] methyl] quinoline-6-carboxamide | 456.2 | 1.73 |
| 711 | 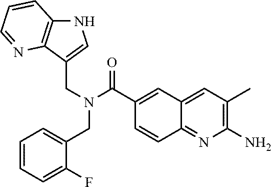 [SMILES: Cc1cc2cc(C(=O)N(Cc3ccccc3F)Cc3c[nH]c4cccnc34)ccc2nc1N] | 2-amino-N-[(2-fluorophenyl)methy1] -3 -methyl-N-(1H-pyrrolo[3,2-b]pyridin-3-ylmethyl)quinoline-6-carboxamide | 440.2 | 1.23 |

Example 833: 2-Amino-N-((3R,4R)-4-methoxytetrahydro-2H-pyran-3-yl)-3-methyl-N-((5-(trifluoromethyl)-2-pyridinyl)methyl)-6-quinolinecarboxamide and 2-amino-N-((3S,4S)-4-methoxytetrahydro-2H-pyran-3-yl)-3-methyl-N-((5-(trifluoromethyl)-2-pyridinyl)methyl)-6-quinolinecarboxamide

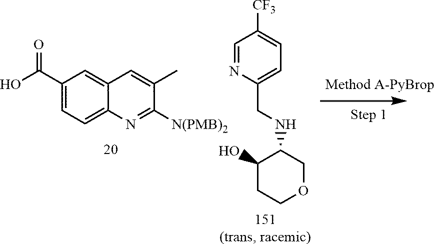

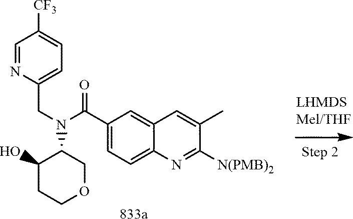

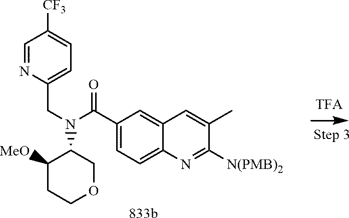

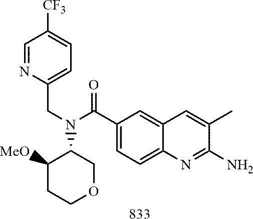

Step 1. 2-(Bis(4-methoxybenzyl)amino)-N-((3R,4R)-4-hydroxytetrahydro-2H-pyran-3-yl)-3-methyl-N-((5-(trifluoromethyl)pyridin-2-yl)methyl)quinoline-6-carboxamide (racemate) (833a) was prepared by Method A-PyBrop in a manner similar to that described for Example 345 from acid 20 and amine 151. The obtained impure product was taken onto the next step without further purification. m/z (ESI): 701.2 (M+H)⁺.

Step 2. To a stirred ice-cooled solution of impure 2-(bis(4-methoxybenzyl)amino)-N-((3R,4R)-4-hydroxytetrahydro-2H-pyran-3-yl)-3-methyl-N-((5-(trifluoromethyl)pyridin-2-yl)methyl)quinoline-6-carboxamide (racemate) (833a) (110 mg, 0.157 mmol) in THF (3.0 mL) was added under nitrogen, lithium bis(trimethylsilyl)amide, 1.0 m in tetrahydrofuran (0.165 mL, 0.165 mmol, Acros Organics). The resulting mixture was stirred at 0° C. for 20 min before iodomethane (22.28 mg, 9.77 µL, 0.157 mmol, Sigma-Aldrich Corporation) was added. The resulting mixture was allowed to warm up to rt and stirred at rt overnight. The reaction was cooled again in ice bath before it was quenched with methanol (1.5 mL). The volatiles were removed and the residue was dissolved in DCM, loaded onto a silica gel precolumn (25 g) and subjected to combi-flash column chromatography on a 12-g ISCO gold column, eluting with EtOAc/heptane to give 20 mg of an impure 2-(bis(4-methoxybenzyl)amino)-N-((3R,4R)-4-methoxytetrahydro-2H-pyran-3-yl)-3-methyl-N-((5-(trifluoromethyl)pyridin-2-yl)methyl)quinoline-6-carboxamide (racemate) (833b) as an off-white solid, which was taken onto the next step. m/z (ESI): 715.2 (M+H)⁺.

Step 3. A mixture of 2-(bis(4-methoxybenzyl)amino)-N-((3R,4R)-4-methoxytetrahydro-2H-pyran-3-yl)-3-methyl-N-((5-(trifluoromethyl)pyridin-2-yl)methyl)quinoline-6-carboxamide (racemate) (833b) (30 mg, 0.042 mmol) and 2,2,2-trifluoroacetic acid (4.79 mg, 4.0 mL, 0.042 mmol, Aldrich) in a microwave reaction vessel was subjected to irradiation for 20 h at 60° C. The volatiles were removed and the residue was dissolved in MeOH and subjected to preparative reverse-phase HPLC (Gemini™ Prep C18 10 µm column; Phenomenex, Torrance, CA; gradient elution of 5 to 85% MeCN in water, where both solvents contain 0.1% TFA 15 min in a 24-min method) to give, after lyophilization, 6 mg of 2-amino-N-((3R,4R)-4-methoxytetrahydro-2H-pyran-3-yl)-3-methyl-N-((5-(trifluoromethyl)pyridin-2-yl)methyl)quinoline-6-carboxamide (racemate) (833) as a white solid as a TFA salt. ¹H NMR (METHANOL-d4, 400 MHz) δ 8.87 (s, 1H), 8.28 (s, 1H), 8.12 (br d, 1H, J=7.3 Hz), 8.02 (s, 1H), 7.89 (br d, 1H, J=9.0 Hz), 7.4-7.8 (m, 2H), 4.9-5.0 (m, 2H), 3.7-4.1 (m, 3H), 3.5-3.7 (m, 2H), 3.3-3.4 (m, 1H), 3.28 (s, 3H), 2.3-2.5 (m, 3H), 2.1-2.3 (m, 1H), 1.0-1.3 (m, 1H). ¹⁹F NMR (METHANOL-d4, 376 MHz) δ -63.84 (s, 3F). m/z (ESI): 475.2 (M+H)⁺.

Example 834: 2-Amino-N-(2-methylpropyl)-3-phenyl-N-((5-(trifluoromethyl)-2-pyridinyl)methyl)-6-quinolinecarboxamide

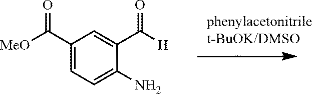

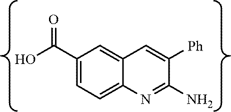

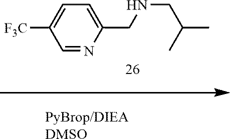

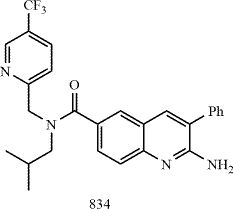

To a stirred mixture in a 10-mL microwave reaction vessel of methyl 4-amino-3-formylbenzoate (55 mg, 0.307 mmol) and phenylacetonitrile (36.0 mg, 0.036 mL, 0.307 mmol, Aldlab Chemicals, LLC) in dimethyl sulfoxide (2 mL) was added at rt 2-methyl-2-propano potassium salt (78 mg, 0.691 mmol, Sigma-Aldrich Corporation). The vessel was sealed, briefly sonicated, and subjected to microwave reaction condition (15 min, 55° C.). To this crude reaction mixture at rt was added 2-methyl-N-((5-(trifluoromethyl)pyridin-2-yl)methyl)propan-1-amine (26) (78 mg, 0.338 mmol), bromotri(pyrrolidin-1-yl)phosphonium hexafluorophosphate(V) (329 mg, 0.706 mmol, Aldrich), and N-ethyl-N-isopropylpropan-2-amine (119 mg, 0.161 mL, 0.921 mmol, Aldrich). The resulting mixture was briefly sonicated and stirred at rt for 15 min when LC-MS showed near completion. The crude mixture was directly submitted to mass directed purification with 0.1% NH₄OH in H₂O (A) and ACN (B) as mobile phase, XBridge column (19×100 mm, 5 um), MS mode: ESI+ to yield 2-amino-N-isobutyl-3-phenyl-N-((5-(trifluoromethyl)pyridin-2-yl)methyl)quinoline-6-carboxamide (50 mg, 0.104 mmol, 34.0 % yield) as a colorless film. ¹H NMR (DMSO-d6, 600 MHz) δ 8.94 (br s, 1H), 8.19 (br s, 1H), 7.8-8.0 (m, 2H), 7.3-7.7 (m, 8H), 6.16 (br s, 2H), 4.7-5.0 (m, 2H), 3.1-3.3 (m, 2H), 1.8-2.2 (m, 1H), 0.92 (br s, 3H), 0.69 (br s, 3H). m/z (ESI): 479.2 (M+H)⁺.

Example 835: 2-Amino-7-hydroxy-3-methyl-N-(2-methylpropyl)-N-((5-(trifluoromethyl)-2-pyridinyl)methyl)-6-quinolinecarboxamide

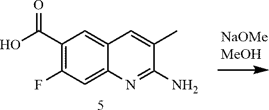

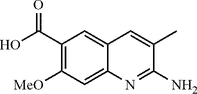 [SMILES: COc1cc2nc(N)c(C)cc2cc1C(=O)O]

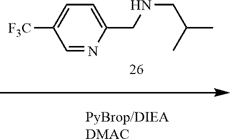

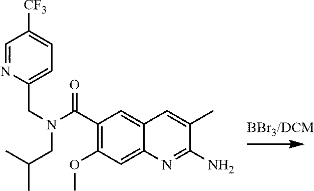 [SMILES: COc1cc2nc(N)c(C)cc2cc1C(=O)N(Cc1ccc(C(F)(F)F)cn1)CC(C)C ; "BBr3/DCM"]

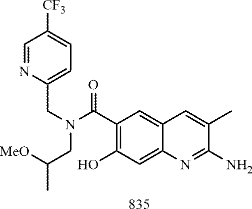

A mixture of 2-amino-7-fluoro-3-methylquinoline-6-carboxylic acid (5) (200 mg, 0.908 mmol) and sodium methanolate, 25 wt.% in MeOH (2835 mg, 3.0 mL, 52.5 mmol, Aldrich) was irradiated under microwave at 120° C. for 2 h. The crude was diluted with water and lyophilized to give 400 mg of crude 2-amino-7-methoxy-3-methylquinoline-6-carboxylic acid as a gray solid, which was used directly in the next step. m/z (ESI): 233.2 (M+H)⁺.

To a stirred mixture of 2-amino-7-methoxy-3-methylquinoline-6-carboxylic acid (100 mg, 0.431 mmol), 2-methyl-N-((5-(trifluoromethyl)pyridin-2-yl)methyl)propan-1-amine (26) (150 mg, 0.646 mmol), and bromotripyrrolidinophosphonium hexafluorophosphate (401 mg, 0.861 mmol, Sigma-Aldrich Corporation) in DMA (2.0 mL) was added N-ethyl-N-isopropylpropan-2-amine (111 mg, 0.150 mL, 0.861 mmol, Aldrich). The resulting mixture was stirred at rt for 1 h. The crude mixture was directly loaded on a silica gel precolumn (25 g) and subjected to combi-flash column chromatography on a 24-g ISCO gold column eluting with MeOH (with 0.5% ammonium hydroxide)/DCM to give 2-amino-N-isobutyl-7-methoxy-3-methyl-N-((5-(trifluoromethyl)pyridin-2-yl)methyl)quinoline-6-carboxamide (77 mg, 0.172 mmol, 40.1% yield) (1) as a white solid. m/z (ESI): 447.20 (M+1)⁺.

To a stirred ice-cooled solution of 2-amino-N-isobutyl-7-methoxy-3-methyl-N-((5-(trifluoromethyl)pyridin-2-yl)methyl)quinoline-6-carboxamide (77 mg, 0.172 mmol) in DMA (2.0 mL) was added tribromoborane, 1.0 M in DCM (0.517 mL, 0.517 mmol, Sigma-Aldrich Corporation) under nitrogen. The resulting mixture was stirred at 0° C. for 20 min and at rt for 30 min before the solvent was stripped off. To the residue, cooled in an ice water bath, was added water (0.2 mL) followed by DMSO (2.5 mL). The resulting clear solution was subjected to preparative reverse-phase HPLC (Gemini™ Prep C18 10 µm column; Phenomenex, Torrance, CA; gradient elution of 10 to 95% MeCN in water, where both solvents contain 0.1% TFA 15 min in a 24-min method) to give, after lyophilization, 2-amino-7-hydroxy-N-isobutyl-3-methyl-N-((5-(trifluoromethyl)pyridin-2-yl)methyl)quinoline-6-carboxamide (835, 20 mg, 0.046 mmol, 26.8 % yield) as a white solid as a TFA salt. ¹H NMR (METHANOL-d4, 400 MHz) δ 8.6-9.0 (m, 1H), 8.15 (br s, 1H), 7.9-8.1 (m, 1H), 7.4-7.9 (m, 2H), 7.0-7.2 (m, 1H), 5.01 (s, 1H), 4.69 (s, 1H), 3.44 (br d, 1H, J=7.5 Hz), 3.18 (br d, 1H, J=7.3 Hz), 2.2-2.4 (m, 3H), 1.9-2.2 (m, 1H), 1.04 (br d, 3H, J=6.5 Hz), 0.78 (br d, 3H, J=6.5 Hz). ¹⁹F NMR (METHANOL-d4, 376 MHz) δ -63.90 (d, 3F, J=13.9 Hz). m/z (ESI): 433.15 (M+1)⁺.

Example 836: (R)-2-amino-N-((3,4-dihydro-2H-pyrano[2,3-c]pyridin-6-yl)methyl)-3-iodo-N-(1-(pyrimidin-2-yl)ethyl)quinoline-6-carboxamide.

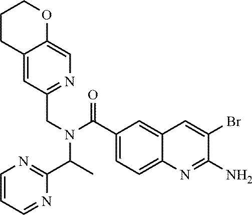 [SMILES: CC(c1ncccn1)N(Cc1cc2c(cn1)OCCC2)C(=O)c1ccc2nc(N)c(Br)cc2c1]

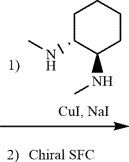

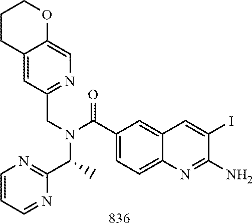

A resealable vial was charged with 2-amino-3-bromo-N-((3,4-dihydro-2H-pyrano[2,3-c]pyridin-6-yl)methyl)-N-(1-(pyrimidin-2-yl)ethyl)quinoline-6-carboxamide (racemic-777, 0.300 g, 0.578 mmol) and sodium iodide (0.26 g, 1.73 mmol, Sigma-Aldrich Corporation) in 1,4-dioxane (6 mL). Then copper(i) iodide (0.02 g, 0.12 mmol, Alfa Aesar) and (1r,2r)-(-)-n,n″-dimethylcyclohexane-1,2-diamine (0.04 mL, 0.23 mmol, Sigma-Aldrich Corporation) were added to the reaction mixture. The vial was sealed, then the reaction mixture was stirred and heated at 120° C. for 48 h. The reaction mixture was diluted with EtOAc and sat. aq. NaHCO₃ The layers were separated and the aqueous layer was extracted with EtOAc (3x). The combined organic extracts were dried over MgSO₄, filtered and concentrated in vacuo. The crude material was absorbed onto a plug of silica gel and purified by chromatography through a Redi-Sep pre-packed silica gel column (80 g), eluting with a gradient of 0-100% EtOAc:EtOH (3:1) in heptane, to provide 2-amino-N-((3,4-dihydro-2H-pyrano[2,3-c]pyridin-6-yl)methyl)-3-iodo-N-(1-(pyrimidin-2-yl)ethyl)quinoline-6-carboxamide (0.165 g, 0.291 mmol, 50% yield) as tan solid.

Previous racemic mixture was purified via preparative SFC using a Chiral Technologies AS column (250 × 21 mm, 5 mm) with a mobile phase of 60% Liquid CO2 and 40% MeOH with 0.2% TEA using a flowrate of 70 mL/min. to generate 75.9 mg of peak 1 (R)-2-amino-N-((3,4-dihydro-2H-pyrano[2,3-c]pyridin-6-yl)methyl)-3-iodo-N-(1-(pyrimidin-2-yl)ethyl)quinoline-6-carboxamide (0.075 g, 0.13 mmol; ee >99%) ¹H NMR (400 MHz, DMSO-d₆) δ ppm 8.54 - 8.81 (m, 3 H), 7.89 (br s, 2 H), 7.67 (br s, 1 H), 7.52 (br s, 1 H), 7.38 (t, J=4.8 Hz, 1 H), 6.95 (br s, 1 H), 6.65 (br s, 2 H), 5.32 (br s, 1 H), 4.75 (br d, J=15.0 Hz, 1 H), 4.58 (br s, 1 H), 4.10 - 4.18 (m, 2 H), 2.61 - 2.80 (m, 2 H), 1.90 (br s, 2 H), 1.58 (br d, J=6.1 Hz, 3 H). m/z (ESI): 567.2 (M+H)⁺. Then peak 2 (S)-2-amino-N-((3,4-dihydro-2H-pyrano[2,3-c]pyridin-6-yl)methyl)-3-iodo-N-(1-(pyrimidin-2-yl)ethyl)quinoline-6-carboxamide (0.072 g, 0.13 mmol; ee >99%). ¹H NMR (400 MHz, DMSO-d₆) δ ppm 8.77 (br d, J=4.8 Hz, 2 H), 8.69 (br s, 1 H), 7.89 (br s, 2 H), 7.67 (br s, 1 H), 7.51 (br s, 1 H), 7.38 (t, J=4.9 Hz, 1 H), 6.95 (br s, 1 H), 6.65 (br s, 2 H), 5.32 (br s, 1 H), 4.75 (br d, J=15.5 Hz, 1 H), 4.58 (br s, 1 H), 4.15 (br t, J=5.0 Hz, 2 H), 2.61 - 2.78 (m, 2 H), 1.90 (br s, 2 H), 1.58 (br d, J=6.1 Hz, 3 H). m/z (ESI)— 567.2 (M+H)⁺.

TABLE 18

Example was prepared in a manner similar to that described above, without chiral SFC since starting material was chiral

| Ex. # | Chemical Structure | Name | LRMS: (ESI + ve ion) m/z |
|---|---|---|---|
| 837 | 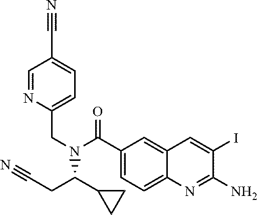 [SMILES: N#CC[C@@H](C1CC1)N(Cc1ccc(C#N)cn1)C(=O)c1ccc2nc(N)c(I)cc2c1] | (S)-2-amino-N-(2-cyano-1- | 523.2 |
| | | cyclopropylethyl)-N-((5- | |
| | | cyanopyridin-2-yl)methyl)-3- | |
| | | iodoquinoline-6-carboxamide | |
| 838 | 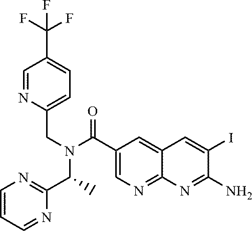 [SMILES: C[C@H](c1ncccn1)N(Cc1ccc(C(F)(F)F)cn1)C(=O)c1cnc2nc(N)c(I)cc2c1] | (R)-7-amino-6-iodo-N-(1- | 580 |
| | | (pyrimidin-2-yl)ethyl)-N-((5- | |
| | | (trifluoromethyl)pyridin-2- | |
| | | yl)methyl)- 1,8-naphthyridine-3- | |
| | | carboxamide | |
| 839 | 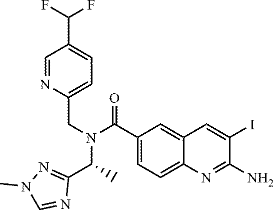 [SMILES: C[C@H](c1ncn(C)n1)N(Cc1ccc(C(F)F)cn1)C(=O)c1ccc2nc(N)c(I)cc2c1] | (R)-2-amino-N-((5- | 564 |
| | | (difluoromethyl)pyridin-2- | |
| | | yl)methyl)-3-iodo-N-(1-(1- | |
| | | methyl-1H-1,2,4-triazol-3- | |
| | | yl)ethyl)quinoline-6- | |
| | | carboxamide | |
| 840 | 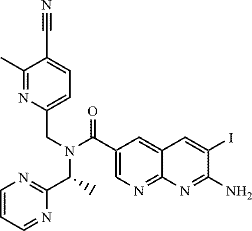 [SMILES: Cc1nc(CN(C(=O)c2cnc3nc(N)c(I)cc3c2)[C@H](C)c2ncccn2)ccc1C#N] | (R)-7-amino-N-((5-cyano-6- | 550.1 |
| | | methylpyridin-2-yl)methyl)-6- | |
| | | iodo-N-(1-(pyrimidin-2- | |
| | | yl)ethy1)-1,8-naphthyridine-3 - | |
| | | carboxamide | |

### Biological Assays

### MTase-Glo Methyl Transferase Assay

Method A: The PRMT5 inhibitory activity of test compounds was determined using the MTase-Glo™ assay (Promega), which monitors the product (S-adenosyl homocysteine or SAH) of methyltransferase reactions. The PRMT5 MTase-Glo assay was conducted in a 384-well white ProxiPlate (PerkinElmer) in a total volume of 12 µL. The PRMT5 enzymatic reaction (in 4 µL) contained 10 nM PRMT5/MEP50 (produced at Amgen), 5 µM S-adenosyl methionine (SAM, Promega), 1 µM Histone H4 (1-21)-Lys(Biotin) (Anaspec), 1 µM 5′-methylthioadenosine (MTA, Sigma) and two-fold serially diluted compounds in a reaction buffer of 50 mM Tris (pH 8.0), 50 mM NaCl, 0.01% Tween 20, 0.01% BSA, and 1 mM DTT. Test compounds were pre-incubated with PRMT5/MEP50, SAM, and MTA for 24 hours before addition of Histone H4 peptide to initiate the PRMT5 reaction. The reaction was allowed to proceed for 2 hours at room temperature and was then terminated by addition of 2 µL of 3X MTase-Glo™ Reagent (Promega) and 150 µM EPZ015666 (Sigma). After a 30-minute incubation at room temperature, 6 µL of MTase-Glo™ Detection Solution (Promega) was added and the plate was incubated at room temperature for an additional 30 minutes. The light signal corresponding to the amount of SAH produced by the PRMT5 reaction was subsequently measured using an Envision multimode reader (PerkinElmer). IC₅₀ and K_{I} values were obtained by analyzing dose-response curves using Genedata Screener (Genedata).

Method B: The PRMT5 inhibitory activity of test compounds was determined using the MTase-Glo™ assay (Promega), which monitors the product (S-adenosyl homocysteine or SAH) of methyltransferase reactions. The PRMT5 MTase-Glo assay was conducted in a 384-well white ProxiPlate (PerkinElmer) in a total volume of 16 µL. The PRMT5 enzymatic reaction (in 8 µL) contained 4 nM PRMT5/MEP50 (Reaction Biology Corp, catalog no.: RD-11-292), 5 µM S-adenosyl methionine (SAM, Promega), 2.5 µM FL-Histone H2A (BPS Bioscience, catalog no.: 52021), 1 µM 5′-methylthioadenosine (MTA, Sigma) and ½ log serially diluted compounds in a reaction buffer of 50 mM Tris (pH 8.0), 50 mM NaCl, 0.01% Tween 20, 0.01% BSA, and 1 mM TCEP. Test compounds were pre-incubated with PRMT5/MEP50, SAM, and MTA for 1 hour before addition of FL-Histone H2A to initiate the PRMT5 reaction. The reaction was allowed to proceed for 20 hours at room temperature and was then terminated by addition of 1 µL of 9X MTase-Glo™ Reagent (Promega). After a 30-minute incubation at room temperature, 8 µL of MTase-Glo™ Detection Solution (Promega) was added and the plate was incubated at room temperature for an additional 30 minutes. The light signal corresponding to the amount of SAH produced by the PRMT5 reaction was subsequently measured using an Envision multimode reader (PerkinElmer). IC₅₀ and K_{I} values were obtained by analyzing dose-response curves using GraphPad Prism®.

### HCT116 Proliferation Assays

HCT116 MTAP null and WT cells were seeded in 96-well tissue culture plates in RPMI 1640 media + 10% fetal bovine serum. Plates were incubated overnight at 37° C. and 5% CO₂. Cells were then treated with a 8- or 9-point serial dilution of compound, using a top concentration of 1 or 10 µM, 1:3 serial dilution steps and, a DMSO-only control. Cells were incubated in the presence of drug for 6 days. Effects on cell viability were measured with the CellTiter-Glo® Luminescent Cell Viability Assay (Promega) per manufacturer’s recommendation. Assay plates were read on an EnVision™ Multilabel Reader using the Ultra-Sensitive luminescence module. IC₅₀ values were calculated with GraphPad Prism v 5.01 using symmetrical sigmoidal dose-response least squares fit with Hill slope fixed to -1 and top constrain to 100% or GeneData Screener using a 4-parameter logistic model to fit dose response curves.

TABLE 19

Biochemical and cellular potency (in HCT116-MTAP null and WT cell line)

| Ex. No | PRMT5 MTase Glo Ki (nM, Meth. A) | PRMT5 MTase Glo Ki (nM, Meth. B) | Protif_ HCT116-MTAP null IC₅₀ (µM) | Prolif_HCT116-WT IC₅₀ (µM) |
|---|---|---|---|---|
| 300 | 0.38 | | 0.317 | 5.3 |
| 301 | 1.22 | | 0.872 | >10 |
| 302 | 0.3 | | 0.359 | >10 |
| 303 | 0.01 | | 0.032 | 0.6 |
| 304 | 0.11 | | 0.182 | 4.1 |
| 305 | 15.1 | | 0.268 | 8.77 |
| 306 | 0.76 | | 0.401 | >10 |
| 307 | 1.88 | | 0.349 | 7.66 |
| 308 | 2.34 | | 0.62 | >10 |
| 309 | 2.35 | | 0.644 | 7.44 |
| 310 | 0.29 | | 0.067 | 1.6 |
| 311 | 0.21 | | 0.163 | 2.84 |
| 312 | 0.36 | | 0.806 | >10 |
| 313 | 0.98 | | 0.902 | >10 |
| 314 | 0.35 | | 0.218 | 5.13 |
| 315 | 0.25 | | 0.181 | 4.34 |
| 316 | 1.03 | | 0.534 | 10.35 |
| 317 | 0.56 | | 0.612 | >10 |
| 318 | 1.91 | | 0.952 | >10 |
| 319 | | | 0.168 | 4.23 |
| 320 | 0.28 | | 0.079 | 3.48 |
| 321 | 0.15 | | 0.288 | >10 |
| 322 | 1.09 | | 0.385 | >10 |
| 323 | 0.36 | | 0.159 | 5.01 |
| 324 | 2 | | 0.849 | >10 |
| 325 | 2.76 | | 0.679 | 6.18 |
| 326 | 0.16 | | 0.125 | 4.32 |
| 327 | 0.35 | | 0.304 | 8.64 |
| 328 | 0.41 | | 0.178 | 4.64 |
| 329 | 0.13 | | 0.128 | 3.9 |
| 330 | 1.87 | | 0.628 | 8.57 |
| 331 | 0.13 | | 0.49 | >10 |
| 332 | 4.87 | | 0.479 | 5.14 |
| 333 | 0.002 | | 0.027 | 0.72 |
| 334 | 0.34 | | 0.434 | >10 |
| 335 | 0.73 | | 0.727 | >10 |
| 336 | 0.47 | | 0.4 | >10 |
| 337 | 0.39 | | 0.434 | >10 |
| 338 | 0.8 | | 0.232 | >10 |
| 339 | | | 0.405 | >10 |
| 340 | 1.39 | | 0.505 | >10 |
| 341 | 0.52 | | 0.437 | >10 |
| 342 | 2.72 | | 0.73 | >10 |
| 343 | 0.66 | | 0.104 | 2.83 |
| 344 | 0.1 | | 0.073 | 1.8 |
| 345 | 0.18 | | 0.123 | 4.05 |
| 346 | 0.04 | | 0.081 | 1.58 |
| 347 | 0.17 | | 0.168 | 6.2 |
| 348 | 0.73 | | 0.413 | >10 |
| 349 | 0.02 | | 0.036 | 0.848 |
| 350 | 0.56 | | 0.397 | >10 |
| 351 | 1.29 | | 0.824 | >10 |
| 352 | 0.14 | | 0.2 | 8.09 |
| 353 | 0.02 | | 0.073 | 1.51 |
| 354 | 0.29 | | 0.378 | >10 |
| 355 | 0.2 | | 0.159 | 3.35 |
| 356 | 0.02 | | 0.035 | 0.801 |
| 357 | 4.34 | | 0.385 | 7.05 |
| 358 | 2.5 | | 0.053 | 1.37 |
| 359 | <0.1 | | 0.208 | 4.74 |
| 360 | 0.08 | | 0.199 | 4.81 |
| 361 | 2.1 | | 0.116 | 3.22 |
| 362 | 0.11 | | 0.106 | 2.46 |
| 363 | 0.2 | | 0.326 | 7.16 |
| 364 | 0.18 | | 0.227 | 6.2 |
| 365 | 0.42 | | 0.391 | >10 |
| 366 | 0.18 | | 0.218 | 7.01 |
| 367 | 0.41 | | 0.282 | >10 |
| 368 | 0.14 | | 0.411 | 7.66 |
| 369 | 0.17 | | 0.138 | 3.4 |
| 370 | 0.79 | | 0.194 | 5.62 |
| 371 | 0.01 | | 0.069 | 1.39 |
| 372 | 0.33 | | 0.124 | 2.41 |
| 373 | 0.03 | | 0.159 | 1.37 |
| | 0.48 | | 0.341 | 8.57 |
| 375 | 0.87 | | 0.064 | 2.13 |
| 376 | 0.02 | | 0.062 | 1.51 |
| 377 | 0.07 | | 0.061 | 2.56 |
| 378 | 0.04 | | 0.34 | 6.96 |
| 379 | 0.89 | | 0.541 | >10 |
| 380 | 0.27 | | 0.079 | 1.67 |
| 381 | 0.33 | | 0.194 | 8.97 |
| 382 | 0.16 | | 0.278 | 9.68 |
| 383 | 2.06 | | 0.434 | 5.32 |
| 384 | 0.01 | | 0.177 | 5.1 |
| 385 | 0.08 | | 0.055 | 1.31 |
| 386 | 3.46 | | 0.241 | 3.83 |
| 387 | 1.67 | | 0.884 | >10 |
| 388 | 0.15 | | 0.137 | 4.88 |
| 389 | 0.37 | | 0.198 | >10 |
| 390 | 0.25 | | 0.023 | 0.46 |
| 391 | 0.01 | | 0.165 | 4.8 |
| 392 | 0.5 | | 0.193 | 6.86 |
| 393 | 0.22 | | 0.342 | 8.21 |
| 394 | 0.12 | | 0.384 | >10 |
| 395 | 0.16 | | 0.082 | 2.37 |
| 396 | 0.27 | | 0.793 | >10 |
| 397 | 0.23 | | 0.286 | 7.96 |
| 398 | 0.58 | | 0.247 | 4.78 |
| 399 | <0.1 | | 0.036 | 0.872 |
| 400 | 0.05 | | 0.041 | 0.96 |
| 401 | 0.08 | | 0.051 | 1.57 |
| 402 | 0.04 | | 0.015 | 0.526 |
| 403 | 0.55 | | 0.263 | 8.88 |
| 404 | 1.68 | | 0.09 | 3.16 |
| 405 | 0.03 | | 0.054 | 1.035 |
| 406 | 0.47 | | 0.682 | >10 |
| 407 | 0.11 | | 0.291 | >10 |
| 408 | 0.13 | | 0.597 | >10 |
| 409 | 0.24 | | 0.692 | >10 |
| 410 | 0.04 | | 0.088 | 5.39 5.39 |
| 411 | 0.22 | | 0.182 | >10 |
| 412 | 0.18 | | 0.123 | 4.05 |
| 413 | 0.65 | | 0.655 | >10 |
| 414 | 0.3 | | 0.234 | 5.18 |
| 415 | 0.54 | | 0.039 | 0.49 |
| 416 | 0.17 | | 0.262 | 6.65 |
| 417 | 0.33 | | 0.151 | >10 |
| 418 | 0.27 | | 0.5 | >10 |
| 419 | 0.16 | | 0.271 | >10 |
| 420 | 0.18 | | 0.225 | 3.45 |
| 421 | 0.54 | | 0.056 | 1.13 |
| 422 | 0.2 | | 0.36 | >10 |
| 423 | 0.07 | | 0.054 | 1.83 |
| 424 | 0.61 | | 0.319 | 9.08 |
| 425 | 0.68 | | 0.38 | 2.17 |
| 426 | 0.47 | | 0.964 | >10 |
| 427 | 0.1 | | 0.151 | 4.25 |
| 428 | 0.08 | | 0.188 | 3.68 |
| 429 | 0.91 | | 0.039 | 0.607 |
| 430 | 0.42 | | 0.506 | >10 |
| 431 | 0.07 | | 0.173 | 3.21 |
| 432 | 0.57 | | 0.024 | 0.11 |
| 433 | 1.26 | | 0.821 | 1.17 |
| 434 | < 0.1 | | 0.049 | 2.42 |
| 435 | 0.08 | | 0.066 | 3.28 |
| 436 | 0.14 | | 0.198 | 3.72 |
| 437 | 0.29 | | 0.076 | 1.35 |
| 438 | 0.76 | | 0.368 | 8.79 |
| 439 | 1.19 | | 0.344 | >10 |
| 440 | < 0.1 | | 0.038 | 0.69 |
| 441 | 0.06 | | 0.107 | 2.81 |
| 442 | 6.87 | | 0.969 | >10 |
| 443 | | | 0.096 | 2.08 |
| 444 | 0.15 | | 0.327 | >10 |
| 445 | 0.53 | | 0.549 | >10 |
| 446 | 0.04 | | 0.045 | 0.851 |
| 447 | 5.51 | | 0.673 | >10 |
| 448 | 0.32 | | 0.238 | 5.23 |
| 449 | 0.27 | | 0.168 | 5.9 |
| 450 | 0.13 | | 0.045 | 2.02 |
| 451 | 0.02 | | 0.013 | 0.232 |
| 452 | 0.06 | | 0.022 | 0.48 |
| 453 | 0.06 | | 0.065 | 1.72 |
| 454 | 0.15 | | 0.252 | 3.94 |
| 455 | 0.16 | | 0.1 | 2.94 |
| 456 | 0.3 | | 0.039 | 0.816 |
| 457 | 0.78 | | 0.717 | >10 |
| 458 | 0.84 | | 0.59 | >10 |
| 459 | 0.25 | | 0.078 | 2.34 |
| 460 | 1.82 | | 0.155 | 7.85 |
| 461 | 1.23 | | 0.322 | 7.78 |
| 462 | 0.29 | | 0.349 | >10 |
| 463 | 1.23 | | 0.683 | |
| 464 | 0.1 | | 0.071 | 1.51 |
| 465 | <0.1 | | 0.024 | |
| 466 | 0.13 | | 0.269 | 4.27 |
| 467 | 0.21 | | 0.028 | 0.248 |
| 468 | < 0.1 | | 0.034 | 0.853 |
| 469 | 0.03 | | 0.033 | 0.592 |
| 470 | 0.09 | | 0.093 | 2.21 |
| 471 | 0.11 | | 0.101 | 3.11 |
| 472 | 1.04 | | 0.889 | >10 |
| 473 | 0.17 | | 0.302 | 5.11 |
| 474 | 0.04 | | 0.088 | 3.27 |
| 475 | 0.07 | | 0.268 | 4.46 |
| 476 | 0.83 | | 0.66 | |
| 477 | 0.11 | | 0.195 | 5.64 |
| 478 | 0.31 | | 0.594 | |
| 479 | 0.09 | | 0.06 | 1.22 |
| 480 | 1.49 | | 0.369 | 1.46 |
| 481 | 0.18 | | 0.225 | 4.45 |
| 482 | 0.1 | | 0.106 | 4.32 |
| 483 | 1.02 | | 0.515 | >10 |
| 484 | 0.11 | | 0.059 | 2.33 |
| 485 | 3.53 | | 0.734 | 4.68 |
| 486 | 0.42 | | 0.123 | 3.83 |
| 487 | 0.04 | | 0.026 | 0.73 |
| 488 | 0.1 | | 0.035 | 1.28 |
| 489 | 0.06 | | 0.027 | |
| 490 | 1.01 | | 0.44 | >10 |
| 491 | 0.1 | | 0.061 | 1.27 |
| 492 | 0.22 | | 0.175 | 3.39 |
| 493 | 0.26 | | 0.146 | 4.08 |
| 494 | 0.66 | | 0.194 | 5.42 |
| 495 | 0.06 | | 0.086 | 3.17 |
| 496 | 0.17 | | 0.023 | 0.462 |
| 497 | 0.19 | | 0.035 | 0.462 |
| 498 | 2.43 | | 0.459 | 2.03 |
| 499 | 0.18 | | 0.251 | >10 |
| 500 | 0.52 | | 0.187 | 4.59 |
| 501 | 0.02 | | 0.107 | 5.92 |
| 502 | 0.06 | | 0.109 | 3.26 |
| 503 | 0.06 | | 0.026 | 0.68 |
| 504 | 0.05 | | 0.049 | 1.36 |
| 505 | 2.51 | | 0.765 | 10.3 |
| 506 | 0.01 | | 0.107 | 1.16 |
| 507 | 0.15 | | 0.046 | 0.913 |
| 508 | 0.73 | | 0.056 | 1.7 |
| 509 | 0.13 | | 0.137 | 4.12 |
| 510 | 0.32 | | 0.07 | 1.91 |
| 511 | 0.14 | | 0.192 | 2.34 |
| 512 | 0.13 | | 0.121 | 3.1 |
| 513 | 2.61 | | 0.552 | >10 |
| 514 | 0.002 | | 0.028 | 0.625 |
| 515 | 0.02 | | 0.023 | 0.48 |
| 516 | 0.14 | | 0.155 | 3.64 |
| 517 | 0.07 | | 0.073 | |
| 518 | 0.11 | | 0.047 | 0.851 |
| 519 | 0.13 | | 0.062 | 1.705 |
| 520 | 0.04 | | 0.024 | 0.737 |
| 521 | 0.01 | | 0.012 | 0.078 |
| 522 | 49.9 | | 0.868 | 5.57 |
| 523 | 0.35 | | 0.334 | 6.38 |
| 524 | | | 0.135 | 2.35 |
| 525 | 0.23 | | 0.598 | 7.61 |
| 526 | 0.33 | | 0.323 | >10 |
| 527 | 0.05 | | 0.01 | 0.142 |
| 528 | 0.14 | | 0.058 | 0.899 |
| 529 | 1.69 | | 0.258 | 4.24 |
| 530 | 0.18 | | 0.108 | 1.71 |
| 531 | 0.1 | | 0.078 | 2.83 |
| 532 | 0.02 | | 0.045 | 0.894 |
| 533 | 0.02 | | 0.019 | 0.755 |
| 534 | 0.33 | | 0.154 | 4.02 |
| 535 | 0.34 | | 0.552 | >10 |
| 536 | 0.02 | | 0.044 | 1.46 |
| 537 | 0.51 | | 0.701 | 5.96 |
| 538 | 0.68 | | 0.732 | >10 |
| 539 | 0.12 | | 0.328 | 7.01 |
| 540 | 0.17 | | 0.194 | 6.27 |
| 541 | 0.13 | | 0.198 | 4.14 |
| 542 | 0.12 | | 0.11 | 4.18 |
| 543 | 0.59 | | 0.568 | >10 |
| 544 | 0.08 | | 0.021 | 0.723 |
| 545 | 0.41 | | 0.032 | 0.478 |
| 546 | 1.09 | | 0.262 | 4.39 |
| 547 | 0.02 | | 0.019 | 0.443 |
| 548 | 0.1 | | 0.088 | 2.08 |
| 549 | 0.07 | | 0.275 | 5.09 |
| 550 | 0.14 | | 0.199 | 5.93 |
| 551 | 0.14 | | 0.021 | 0.634 |
| 552 | 0.32 | | 0.22 | 2.55 |
| 553 | 0.36 | | 0.454 | >10 |
| 554 | 0.07 | | 0.122 | 4.28 |
| 555 | 0.07 | | 0.125 | 3.82 |
| 556 | 4.1 | | 0.342 | 10.1 |
| 557 | 0.18 | | 0.042 | 1.73 |
| 558 | 0.08 | | 0.038 | 1.3 |
| 559 | 0.07 | | 0.147 | 2.19 |
| 560 | 0.2 | | 0.623 | >10 |
| 561 | 0.3 | | 0.895 | >10 |
| 562 | | 3.76 | 0.455 | >1 |
| 563 | | | 0.165 | >1 |
| 564 | | | 0.102 | >1 |
| 565 | | 2.77 | 0.094 | 3.49 |
| 566 | | 4.58 | 0.277 | >10 |
| 567 | | 4.44 | 0.103 | >1 |
| 568 | | 2.-2 | 0.13 | 5.55 |
| 569 | | | 0.331 | 7.16 |
| 570 | | 2.39 | 0.343 | >10 |
| 571 | | | 0.202 | >1 |
| 572 | | 3.48 | 0.608 | >10 |
| 573 | | | 0.76 | >1 |
| 574 | | | 0.49 | >1 |
| 575 | | 2.89 | 0.078 | >1 |
| 576 | | 5.89 | 0.749 | >10 |
| 577 | | 3.89 | 0.159 | 6.65 |
| 578 | | 15.4 | 0.859 | 5.09 |
| 579 | | - | 0.095 | >1 |
| 580 | | 2.24 | 0.11 | 5.6 |
| 581 | | 16 | 0.84 | >1 |
| 582 | | | 0.49 | >1 |
| 583 | | | 0.14 | >1 |
| 584 | | | 0.29 | >1 |
| 585 | | | 0.58 | 11 |
| 586 | | | 0.344 | 5.55 |
| 587 | | | 0.807 | 0.404 |
| 588 | | | 0.76 | >1 |
| 589 | | | 0.549 | >1 |
| 590 | | 14.9 | 0.921 | 6.8 |
| 591 | | 16.5 | 0.456 | >1 |
| 592 | | 2-0.9 | 0.407 | >1 |
| 593 | | 13.2 | 0.185 | >1 |
| 594 | | 26 | 0.64 | >1 |
| 595 | | 21.5 | 0.99 | 8.2 |
| 596 | | 17.1 | 0.352 | >10 |
| 597 | | 6.35 | 0.3 | >1 |
| 598 | | 2.44 | 0.18 | >1 |
| 599 | | 3.12 | 0.28 | >1 |
| 600 | | 1.83 | 0.044 | >1 |
| 601 | | 1.78 | 0.038 | 0.82 |
| 602 | | 7.26 | 0.085 | >1 |
| 603 | | 23.7 | 0.317 | >1.5 |
| 604 | | 5.46 | 0.044 | 0.4 |
| 605 | | 6.2 | 0.145 | >1.3 |
| 606 | | 2.9 | 0.027 | 0.28 |
| 607 | | 7.26 | 0.37 | >3.2 |
| 608 | | 5.26 | 0.4 | >1 |
| 609 | | 26.8 | 0.54 | >1 |
| 610 | | 9.6 | 0.54 | >1 |
| 611 | | 3.-7 | 0.07 | >1 |
| 612 | | 7.56 | 0.34 | >1 |
| 613 | | 4.35 | 0.269 | 3.9 |
| 614 | | 9.42 | 0.95 | >1 |
| 615 | | 1.62 | 0.276 | 3.5 |
| 616 | | 3.29 | 0.32 | >1 |
| 617 | | 1.72 | 0.286 | >1 |
| 618 | | 1.59 | 0.055 | 1.4 |
| 619 | | 1.87 | 0.128 | 1.3 |
| 620 | | 1.83 | 0.1 | >1 |
| 621 | | 3.97 | 0.133 | >1 |
| 622 | | 1.98 | 0.13 | >1 |
| 623 | | 3.14 | 0.056 | 0.9 |
| 624 | | 5.19 | 0.187 | >1 |
| 625 | | 1.99 | 0.18 | |
| 626 | | 42.7 | | |
| 627 | | 2.8 | 0.36 | |
| 628 | | 3.-4 | 0.053 | 0.44 |
| 629 | | 5.93 | 0.77 | |
| 630 | | 4.58 | 0.033 | |
| 631 | | 7.14 | 0.028 | |
| 632 | | 5.3 | 0.34 | |
| 633 | | 4.8 | 0.33 | |
| 634 | | 6.84 | 0.93 | |
| 635 | | 1.29 | 0.018 | |
| 636 | | 2.3 | 0.89 | |
| 637 | | 5.2 | >1 | |
| 638 | | 1.9 | 0.2 | |
| 639 | | 12.2 | 0.58 | |
| 640 | | 14.1 | 0.73 | |
| 641 | | 13.1 | 0.92 | |
| 642 | | 2.55 | 0.086 | |
| 643 | | 39.6 | | |
| 644 | | 29.3 | | |
| 645 | | 5.93 | >1 | |
| 646 | | 6.83 | 0.85 | |
| 647 | | 4.11 | 0.32 | |
| 648 | | 1.68 | 0.18 | |
| 649 | | 2.69 | 0.31 | |
| 650 | | 2.21 | 0.37 | |
| 651 | | 3.-9 | 0.31 | |
| 652 | | 3.-7 | 0.16 | |
| 653 | | 2.79 | 0.28 | |
| 654 | | 5.61 | 0.25 | |
| 655 | | 4.-9 | 0.55 | |
| 656 | | 5.28 | 0.21 | |
| 657 | | 5.34 | 0.62 | |
| 658 | | 12.6 | | |
| 659 | | 4.33 | 0.251 | |
| 660 | | 7.14 | 0.8 | |
| 661 | | 1.81 | 0.069 | |
| 662 | | 3.58 | 0.18 | |
| 663 | | 1.7 | 0.037 | |
| 664 | | 9.1 | 0.67 | |
| 665 | | 9.17 | 0.68 | |
| 666 | | 3.93 | 0.041 | |
| 667 | | 3.9 | 0.024 | |
| 668 | | 4.69 | 0.27 | >1 |
| 669 | | 8.5 | 0.82 | |
| 670 | | 5.15 | 0.86 | |
| 671 | | 6.82 | 0.18 | |
| 672 | | 5.56 | 0.404 | >1 |
| 673 | | 5.32 | 0.095 | |
| 674 | | 15.3 | 0.51 | |
| 675 | | 5.-4 | 0.232 | >1 |
| 676 | | 3.88 | 0.255 | >1 |
| 677 | | 9.37 | 0.95 | |
| 678 | | 5.57 | 0.5 | |
| 679 | | 36.8 | | |
| 680 | | 1.76 | 0.157 | 7.7 |
| 681 | | 4.87 | 0.24 | |
| 682 | | 23 | | |
| 683 | | 4.29 | 0.297 | >1 |
| 684 | | 2.29 | 0.114 | >1 |
| 685 | | 1.22 | 0.051 | |
| 686 | | 5.12 | 0.59 | |
| 687 | | 1.56 | 0.11 | |
| 688 | | 1.41 | 0.24 | |
| 689 | | 2.94 | 0.33 | |
| 690 | | 1.68 | 0.094 | |
| 691 | | 3.51 | 0.52 | |
| 692 | | 1-0.7 | >1 | |
| 693 | | 6.14 | 0.097 | |
| 694 | | 5.62 | 0.224 | 8.6 |
| 695 | | 6.3 | 0.76 | |
| 696 | | 2.19 | 0.2 | |
| 697 | | 36.5 | | |
| 698 | | 36.4 | | |
| 699 | | 3.86 | 0.05 | >1 |
| 700 | | 38.1 | | |
| 701 | | 37.2 | | |
| 702 | | 5.12 | 0.046 | 0.56 |
| 703 | | 5.62 | 0.222 | 2.3 |
| 704 | | 25.1 | 0.08 | >1 |
| 705 | | 9.63 | 0.76 | >1 |
| 706 | | 31 | | |
| 708 | | 2.79 | 0.155 | >1 |
| 709 | | 2.97 | 0.334 | 1.43 |
| 710 | | 4.13 | 0.177 | 1.14 |
| 711 | | | 0.279 | 9.78 |
| 724 | | | 0.136 | >1 |
| 725 | | | 0.252 | >1 |
| 726 | | | 0.313 | >1 |
| 727 | | | 0.256 | >1 |
| 728 | | | 0.264 | 6.81 |
| 729 | | | 0.114 | 6.902 |
| 730 | | | 0.507 | 8.46 |
| 731 | | | 0.05 | 0.693 |
| 732 | | | 0.231 | >10 |
| 733 | | | 0.434 | >10 |
| 734 | | | 0.994 | >10 |
| 735 | | | 0.22 | >10 |
| 736 | | | 0.151 | 6.96 |
| 737 | | | 0.148 | 6.8 |
| 738 | | | 0.173 | 4.3 |
| 739 | | | 0.355 | >10 |
| 740 | | | 0.08 | 4.4 |
| 741 | | | 0.27 | 8.36 |
| 742 | | | 0.04 | 1.17 |
| 743 | | | 0.054 | 0.694 |
| 744 | | | 0.188 | 4.125 |
| 745 | | | 0.223 | 1.2445 |
| 746 | | | 0.139 | 7.26 |
| 747 | | | 0.385 | 5.26 |
| 748 | | | 0.084 | 2.62 |
| 749 | | | 0.095 | 2.15 |
| 750 | | | 0.177 | 3.145 |
| 751 | | | 0.144 | 1.74 |
| 752 | | | 0.119 | 1.74 |
| 753 | | | 0.408 | >10 |
| 754 | | | 0.13 | 4.11 |
| 755 | | | 0.184 | >10 |
| 756 | | | 0.228 | 5.95 |
| 757 | | | 0.313 | 8.87 |
| 758 | | | 0.337 | >10 |
| 759 | | | 0.293 | >10 |
| 760 | | | 0.1 | 2.93 |
| 761 | | | 0.064 | 1.35 |
| 762 | | | 0.18 | 8.795001 |
| 763 | | | 0.057 | 4.34 |
| 764 | | | 0.287 | 4.22 |
| 765 | | | 0.18 | 0.51225 |
| 766 | | | 0.161 | 5.9 |
| 767 | | | 0.056 | 3.546667 |
| 768 | | | 0.116 | 3.96 |
| 769 | | | 0.052 | 5.01 |
| 770 | | | 0.132 | 4.75 |
| 771 | | | 0.27 | >10 |
| 772 | | | 0.437 | 17.55 |
| 773 | | | 0.304 | >10 |
| 774 | | | 0.139 | 5.21 |
| 775 | | | 0.05 | 1.68 |
| 776 | | | 0.195 | 7.225 |
| 777 | | | 0.372 | >10 |
| 778 | | | 0.072 | 2.05 |
| 780 | | | 0.144 | 1.8 |
| 781 | | | 0.26 | 3.395 |
| 782 | | | 0.017 | 0.46 |
| 784 | | | 0.261 | 4.7 |
| 785 | | | 0.123 | 2.605 |
| 786 | | | 0.322 | >10 |
| 787 | | | 0.059 | 1.735 |
| 788 | | | 0.064 | 1.59 |
| 789 | | | 0.162 | 6.15 |
| 790 | | | 0.156 | 5.16 |
| 791 | | | 0.06 | 1.72 |
| 792 | | | 0.119 | 3.84 |
| 793 | | | 0.783 | >10 |
| 795 | | | 0.056 | 1.39 |
| 796 | | | 0.308 | >10 |
| 797 | | | 0.229 | 3.82 |
| 798 | | | 0.25 | 6.96 |
| 799 | | | 0.013 | 0.301 |
| 800 | | | 0.173 | 2.79 |
| 801 | | | 0.533 | >10 |
| 802 | | | 0.393 | 9.3 |
| 803 | | | 0.412 | >10 |
| 804 | | | 0.226 | 5.77 |
| 805 | | | 0.144 | 2 |
| 806 | | | 0.552 | 6.8 |
| 807 | | | 0.075 | 0.764 |
| 808 | | | 0.454 | 19.9 |
| 809 | | | 0.015 | 0.362 |
| 810 | | | 0.594 | 5.4 |
| 811 | | | 0.422 | >10 |
| 812 | | | 0.043 | 1.04 |
| 813 | | | 0.098 | 2.63 |
| 814 | | | 0.062 | 1.76 |
| 815 | | | 0.25 | 5.15 |
| 816 | | | 0.018 | 0.368 |
| 817 | | | 0.369 | 1.31 |
| 818 | | | 0.024 | 0.372 |
| 819 | | | 0.029 | 0.543 |
| 820 | | | 0.239 | 3.55 |
| 821 | | | 0.019 | 0.3715 |
| 822 | | | 0.045 | 0.763 |
| 823 | | | 0.041 | 0.695 |
| 824 | | | 0.607 | >10 |
| 827 | | | 0.88 | >1 |
| 828 | | | 0.91 | >10 |
| 829 | | | 0.84 | >10 |
| 830 | | | 0.38 | >10 |
| 831 | | | 0.44 | >10 |
| 832 | | | 0.38 | >10 |
| 833 | | | 0.039 | 1.19 |
| 834 | | | 0.523 | 3.19 |
| 835 | | | 0.448 | 8.01 |
| 836 | | | 0.129 | >10 |
| 837 | | | 0.042 | 2.17 |
| 838 | | | 0.056 | 1.65 |
| 839 | | | 0.057 | 1.99 |
| 840 | | | 0.09 | 5.5 |

All publications and patent applications cited in this specification are hereby incorporated by reference herein in their entireties and for all purposes as if each individual publication or patent application were specifically and individually indicated as being incorporated by reference and as if each reference was fully set forth in its entirety. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed:

1. A compound of Formula I 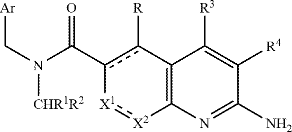
a tautomer thereof, a stereoisomer thereof, or a pharmaceutically acceptable salt of any of the foregoing,
wherein
$$\overline{- - - - - -}$$
represents a single or double bond;
X¹ and X² are both in each instance independently N or C; wherein if X¹ is C it can be optionally substituted with halo or C₁₋₆alkyl;
Ar is a six membered aromatic ring having 0-2 N atoms, wherein each Ar could be independently substituted with 0-2 R^{a} groups;
wherein R^{a} is in each instance independently selected from cyano, halo, optionally substituted C₁₋₆alkyl, C₁₋₆haloalkyl, OR^{b}, NR^{c}R^{d}, —C(O)NR^{c}R^{d}, =S, —SO₂, —SO₂C₁_{—6}alkyl, —C(O), —C(O)C₁₋₆ alkyl, C(O)OC₁₋₆ alkyl, difluoro-pyrrolidinyl, and 4 to 6-membered heterocyclic ring, with 0-2 heteroatoms independently selected from O and N, and which heterocyclic ring can be further independently substituted with 0-2 halogen, C₁₋₆ alkyl, —C(O)H, —C(O)C₁₋₆ alkyl or optionally substituted cycloalcoxyl;
wherein each R^{b} is in each instance independently selected from H, optionally substituted C₁₋₆ alkyl, wherein the substituents can be selected from halo; or oxetanyl;
wherein each R^{c} and R^{d} is independently selected from H, C₁₋₃alkyl, C₁₋₃ haloalkyl or —CO;
wherein R^{e} in each instance is selected from H or C₁₋₆alkyl;
wherein R^{f} and R^{g} in each instance is independently selected from H and C₁₋₆alkyl;
wherein R is H or methyl;
wherein R¹ and R² are in each instance is independently selected from H, optionally substituted C₁₋₆ alkyl, optionally substituted C₁₋₆ alkynyl, —C(OR^{e}), optionally substituted single and double cyclyl having 0-3 N, S or O atoms; wherein the substituents are selected from halo, optionally substituted —C(O)NR^{f}R^{g}, OH and an optionally substituted 5-membered ring having 0-3 N atoms;
or R¹ and R² and the carbon atom to which they are attached can form an optionally substituted single or double carbocyclic or heterocyclic ring, which may be saturated, partially saturated or aromatic and further wherein the heterocyclic ring includes 1, 2 or 3 heteroatoms independently selected from N, O, and S;
wherein the substituents are selected from the group of optionally substituted C₁₋₆ alkyl, halo, CN, OR^{e} and —C(OR^{e}),
provided that R¹ and R² are not both H at the same time;
and wherein R³ and R⁴ are in each instance independently selected from H, halogen, alkynyl, cyano and C₁₋₆ alkyl, optionally substituted with halo or deuterium.

2. The compound of claim 1, the tautomer thereof, the stereoisomer thereof, or the pharmaceutically acceptable salt of any of the foregoing, wherein wherein X¹ is C.

3. The compound of claim 2, the tautomer thereof, the stereoisomer thereof, or the pharmaceutically acceptable salt of any of the foregoing, wherein X² is C.

4. The compound of claim 3, the tautomer thereof, the stereoisomer thereof, or the pharmaceutically acceptable salt of any of the foregoing, wherein the compound is of Formula IA

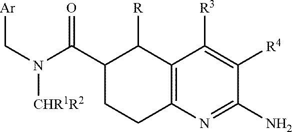

5. The compound of claim 1, the tautomer thereof, the stereoisomer thereof, or the pharmaceutically acceptable salt of any of the foregoing, wherein the compound is of Formula IB

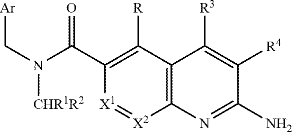

6. The compound of claim 2, the tautomer thereof, the stereoisomer thereof, or the pharmaceutically acceptable salt of any of the foregoing, wherein X² is N.

7. The compound of claim 1, the tautomer thereof, the stereoisomer thereof, or the pharmaceutically acceptable salt of any of the foregoing, wherein X¹ is N.

8. The compound of claim 1, the tautomer thereof, the stereoisomer thereof, or the pharmaceutically acceptable salt of any of the foregoing, wherein Ar is pyridinyl.

9. The compound of claim 1, the tautomer thereof, the stereoisomer thereof, or the pharmaceutically acceptable salt of any of the foregoing, wherein Ar is pyradazinyl.

10. The compound of claim 1, the tautomer thereof, the stereoisomer thereof, or the pharmaceutically acceptable salt of any of the foregoing, wherein Ar is pyrazinyl.

11. The compound of claim 1, the tautomer thereof, the stereoisomer thereof, or the pharmaceutically acceptable salt of any of the foregoing, wherein Ar is phenyl.

12. The compound of claim 1, the tautomer thereof, the stereoisomer thereof, or the pharmaceutically acceptable salt of any of the foregoing, wherein R¹ and R² are wach independently selected from methyl and pyrimidinyl.

13. The compound of claim 1, the tautomer thereof, the stereoisomer thereof, or the pharmaceutically acceptable salt of any of the foregoing, wherein R¹ and R² are each independently selected from methyl and pyridinyl.

14. The compound of claim 1, the tautomer thereof, the stereoisomer thereof, or the pharmaceutically acceptable salt of any of the foregoing, wherein R¹ and R² are each independently selected from H, methyl, C₁₋₆alkyl and an optionally substituted single or double cyclyl having 0-3 N, S or O items.

15. The compound of claim 14, the tautomer thereof, the stereoisomer thereof, or the pharmaceutically acceptable salt of any of the foregoing, wherein the cyclyl is optionally substituted thiophenyl, optionally substituted thiazolyl or optionally substituted oxazolyl.

16. The compound of claim 1, the tautomer thereof, the stereoisomer thereof, or the pharmaceutically acceptable salt of any of the foregoing, R¹ and R² and the carbon atom to which they are attached can form an optionally substituted single or double carbocyclic or heterocyclic ring, which may be saturated, partially saturated or aromatic and further wherein the heterocyclic ring includes 1, 2 or 3 heteroatoms independently selected from N, O, and S.

17. The compound of claim 1, the tautomer thereof, the stereoisomer thereof, or the pharmaceutically acceptable salt of any of the foregoing, wherein R⁴ is methyl or halogen.

18. The compound of claim 1, the tautomer thereof, the stereoisomer thereof, or the pharmaceutically acceptable salt of any of the foregoing, wherein R^{a} is cyano, halo or optionally substituted C₁₋₆alkyl.

19. A compound, the tautomer thereof, the stereoisomer thereof, or the pharmaceutically acceptable salt of any of the foregoing, selected from:
2-amino-3-methyl-N-(2-methylpropyl)-N-((5-(trifluoromethyl)-2-pyridinyl)methyl)-6-quinolinecarboxamide,
2-amino-3-methyl-N-((2R)-3,3,3-trifluoro-2-methylpropyl)-N-((5-(trifluoromethyl)-2-pyridinyl)methyl)-6-quinolinecarboxamide,
2-amino-3-methyl-N-((2S)-3,3,3-trifluoro-2-methylpropyl)-N-((5-(trifluoromethyl)-2-pyridinyl)methyl)-6-quinolinecarboxamide,
2-amino-3-methyl-N-((2S)-3,3,3-trifluoro-2-methoxypropyl)-N-((5-(trifluoromethyl)-2-pyridinyl)methyl)-6-quinolinecarboxamide,
2-amino-3-methyl-N-((7R)-4,5,6,7-tetrahydro-1H-indazol-7-yl)-N-((5-(trifluoromethyl)-2-pyridinyl)methyl)-6-quinolinecarboxamide,
2-amino-N-((1R)-1-(6-cyano-2-pyridinyl)ethyl)-3-methyl-N-((5-(trifluoromethyl)-2-pyridinyl)methyl)-6-quinolinecarboxamide,
2-amino-N-((6-methoxy-3-pyridazinyl)methyl)-3-methyl-N-((1-R)-1-(1-methyl-1H-1,2,4-triazol-3 -yl)ethyl)-6-quinolinecarboxamide,
2-amino-N-((3R,4R)-4-methoxytetrahydro-2H-pyran-3-yl)-3-methyl-N-((5-(trifluoromethyl)-2-pyridinyl)methyl)-6-quinolinecarboxamide,
2-amino-N-((3S,4R)-3-fluorotetrahydro-2H-pyran-4-yl)-3-methyl-N-((5-(trifluoromethyl)-2-pyridinyl)methyl)-6-quinolinecarboxamide,
7-amino-6-bromo-N-((1R)-1-(1,3-thiazol-4-yl)ethyl)-N-((6-(trifluoromethyl)-3-pyridazinyl)methyl)-1,8-naphthyridine-3-carboxamide,
2-amino-N-((3S,4R)-3-hydroxytetrahydro-2H-pyran-4-yl)-3-methyl-N-((5-(trifluoromethyl)-2-pyridinyl)methyl)-6-quinolinecarboxamide,
2-amino-N-((3S,4R)-3-methoxytetrahydro-2H-pyran-4-yl)-3-methyl-N-((5-(trifluoromethyl)-2-pyridinyl)methyl)-6-quinolinecarboxamide,
2-amino-3-methyl-N-((1R)-1-(1,2,4-oxadiazol-3-yl)ethyl)-N-((5-(trifluoromethyl)-2-pyridinyl)methyl)-6-quinolinecarboxamide,
2-amino-N-((1R,2R)-2-cyanocyclopentyl)-3-methyl-N-((5-(trifluoromethyl)-2-pyridinyl)methyl)-6-quinolinecarboxamide,
2-amino-3-methyl-N-(4-pyrimidinylmethyl)-N-((5-(trifluoromethyl)-2-pyridinyl)methyl)-6-quinolinecarboxamide,
2-amino-3-methyl-N-(2-pyrazinylmethyl)-N-((5-(trifluoromethyl)-2-pyridinyl)methyl)-6-quinolinecarboxamide,
2-amino-3-methyl-N-((1-methyl-1H-1,2,4-triazol-3-yl)methyl)-N-((5-(trifluoromethyl)-2-pyridinyl)methyl)-6-quinolinecarboxamide,
2-amino-7-fluoro-3-methyl-N-(2-methylpropyl)-N-((5-(trifluoromethyl)-2-pyridinyl)methyl)-6-quinolinecarboxamide,
2-amino-3-methyl-N-((5-methyl-2-pyrazinyl)methyl)-N-((5-(trifluoromethyl)-2-pyridinyl)methyl)-6-quinolinecarboxamide,
2-amino-7-fluoro-3-methyl-N-(2-pyrazinylmethyl)-N-((5-(trifluoromethyl)-2-pyridinyl)methyl)-6-quinolinecarboxamide,
2-amino-7-fluoro-3-methyl-N-((5-methyl-1,2-oxazol-3-yl)methyl)-N-((5-(trifluoromethyl)-2-pyridinyl)methyl)-6-quinolinecarboxamide,
2-amino-3-methyl-N-((1R)-1-(2-pyrazinyl)ethyl)-N-((5-(trifluoromethyl)-2-pyridinyl)methyl)-6-quinolinecarboxamide,
2-amino-3-methyl-N-(2-pyrimidinylmethyl)-N-((5-(trifluoromethyl)-2-pyridinyl)methyl)-6-quinolinecarboxamide,
2-amino-N-((3-fluoro-5-(trifluoromethyl)-2-pyridinyl)methyl)-3-methyl-N-(2-methylpropyl)-6-quinolinecarboxamide,
2-amino-N-((5-chloro-2-pyrimidinyl)methyl)-3-methyl-N-((5-(trifluoromethyl)-2-pyridinyl)methyl)-6-quinolinecarboxamide,
2-amino-3-bromo-N-(2-pyrimidinylmethyl)-N-((5-(trifluoromethyl)-2-pyridinyl)methyl)-6-quinolinecarboxamide,
2-amino-3-bromo-N-(2-methylpropyl)-N-((5-(trifluoromethyl)-2-pyridinyl)methyl)-6-quinolinecarboxamide,
2-amino-7-fluoro-3-methyl-N-(2-pyrimidinylmethyl)-N-((5-(trifluoromethyl)-2-pyridinyl)methyl)-6-quinolinecarboxamide,
2-amino-N-((5-chloro-2-pyridinyl)methyl)-3-methyl-N-(2-pyrimidinylmethyl)-6-quinolinecarboxamide,
2-amino-N-((5-fluoro-2-pyrimidinyl)methyl)-3-methyl-N-((5-(trifluoromethyl)-2-pyridinyl)methyl)-6-quinolinecarboxamide,
2-amino-N-((3-fluoro-2-pyridinyl)methyl)-3-methyl-N-((5-(trifluoromethyl)-2-pyridinyl)methyl)-6-quinolinecarboxamide,
7-amino-6-bromo-N-((3-fluoro-2-pyridinyl)methyl)-N-((5-(trifluoromethyl)-2-pyridinyl)methyl) -1,8-naphthyridine-3 -carboxamide,
7-amino-N-((3-fluoro-2-pyridinyl)methyl)-6-methyl-N-((5-(trifluoromethyl)-2-pyridinyl)methyl) -1,8-naphthyridine-3 -carboxamide,
2-amino-N-((5-(difluoromethyl)-2-pyridinyl)methyl)-3-methyl-N-(2-methylpropyl)-6-quinolinecarboxamide,
2-amino-3-methyl-N-((1R)-1-(2-pyrimidinyl)ethyl)-N-((5-(trifluoromethyl)-2-pyridinyl)methyl)-6-quinolinecarboxamide,
2-amino-N-((3-fluoro-2-pyridinyl)methyl)-3-methyl-N-((6-(trifluoromethyl)-3-pyridinyl)methyl)-6-quinolinecarboxamide,
2-amino-N-((4R)-3,4-dihydro-2H-pyrano[3,2-b]pyridin-4-yl)-3-methyl-N-((5-(trifluoromethyl)-2-pyridinyl)methyl)-6-quinolinecarboxamide,
7-amino-6-methyl-N-((1R)-1-(2-pyrazinyl)ethyl)-N-((5-(trifluoromethyl)-2-pyridinyl)methyl) -1,8-naphthyridine-3 -carboxamide,
2-amino-N-(cyclopropylmethyl)-3-methyl-N-((5-(trifluoromethyl)-2-pyridinyl)methyl)-6-quinolinecarboxamide,
2-amino-N-((5-bromo-2-pyridinyl)methyl)-3-methyl-N-(2-methylpropyl)-6-quinolinecarboxamide,
2-amino-N-((3,5-difluoro-2-pyridinyl)methyl)-3-methyl-N-((5-(trifluoromethyl)-2-pyridinyl)methyl)-6-quinolinecarboxamide,
2-amino-N-((1R)-1-(3-fluoro-2-pyridinyl)ethyl)-3-methyl-N-((5-(trifluoromethyl)-2-pyridinyl)methyl)-6-quinolinecarboxamide,
2-amino-N-((5-(difluoromethyl)-2-pyridinyl)methyl)-N-((3-fluoro-2-pyridinyl)methyl)-3-methyl-6-quinolinecarboxamide,
2-amino-N-((1 S)-1-(3-fluoro-2-pyridinyl)ethyl)-3-methyl-N-((5-(trifluoromethyl)-2-pyridinyl)methyl)-6-quinolinecarboxamide,
2-amino-N-((3,5-difluoro-2-pyridinyl)methyl)-N-((3-fluoro-2-pyridinyl)methyl)-3-methyl-6-quinolinecarboxamide,
2-amino-N-((5-cyclopropyl-2-pyridinyl)methyl)-3-methyl-N-(2-methylpropyl)-6-quinolinecarboxamide,
2-amino-N-((1R)-1-(5-fluoro-2-pyridinyl)ethyl)-3-methyl-N-((5-(trifluoromethyl)-2-pyrazinyl)methyl)-6-quinolinecarboxamide,
2-amino-3-methyl-N-((1R)-1-(2-pyrimidinyl)ethyl)-N-((5-(trifluoromethyl)-2-pyrazinyl)methyl)-6-quinolinecarboxamide,
7-amino-6-bromo-N-((1R)-1-(3-fluoro-2-pyridinyl)ethyl)-N-((5-(trifluoromethyl)-2-pyridinyl)methyl) -1,8-naphthyridine-3 -carboxamide,
7-amino-N-((1R)-1-(3-fluoro-2-pyridinyl)ethyl)-6-methyl-N-((5-(trifluoromethyl)-2-pyridinyl)methyl) -1,8-naphthyridine-3 -carboxamide,
2-amino-3-methyl-N-((8R)-5,6,7,8-tetrahydro-8-quinolinyl)-N-((5-(trifluoromethyl)-2-pyridinyl)methyl)-6-quinolinecarboxamide,
2-amino-3-methyl-N-((8R)-5,6,7,8-tetrahydro-8-isoquinolinyl)-N-((5-(trifluoromethyl)-2-pyridinyl)methyl)-6-quinolinecarboxamide,
2-amino-N-((1R)-1-(3-fluoro-2-pyridinyl)ethyl)-N-((3-fluoro-5-(trifluoromethyl)-2-pyridinyl)methyl)-3-methyl-6-quinolinecarboxamide,
2-amino-N-((1R)-1-(5-fluoro-2-pyridinyl)ethyl)-3-methyl-N-((5-(trifluoromethyl)-2-pyridinyl)methyl)-6-quinolinecarboxamide,
2-amino-N-((1S)-1-(5-fluoro-2-pyridinyl)ethyl)-3-methyl-N-((5-(trifluoromethyl)-2-pyridinyl)methyl)-6-quinolinecarboxamide,
2-amino-3-methyl-N-((1R)-1-(5-methyl-1,2-oxazol-3-yl)ethyl)-N-((5-(trifluoromethyl)-2-pyridinyl)methyl)-6-quinolinecarboxamide,
2-amino-N-((3-fluoro-2-pyridinyl)methyl)-3-methyl-N-((1R)-1-(5-(trifluoromethyl)-2-pyridinyl)ethyl)-6-quinolinecarboxamide,
2-amino-N-((1R)-1-cyclopropylethyl)-3-methyl-N-((5-(trifluoromethyl)-2-pyridinyl)methyl)-6-quinolinecarboxamide,
2-amino-3-methyl-N-(2-methylpropyl)-N-((1R)-1-(5-(trifluoromethyl)-2-pyridinyl)ethyl)-6-quinolinecarboxamide,
2-amino-N-((5-cyano-2-pyridinyl)methyl)-N-((3-fluoro-2-pyridinyl)methyl)-3-methyl-6-quinolinecarboxamide,
2-amino-N-((4R)-3,4-dihydro-2H-pyrano[2,3-b]pyridin-4-yl)-3-methyl-N-((5-(trifluoromethyl)-2-pyridinyl)methyl)-6-quinolinecarboxamide,
2-amino-N-((5-bromo-2-pyridinyl)methyl)-N-((1R)-1-(3-fluoro-2-pyridinyl)ethyl)-3-methyl-6-quinolinecarboxamide,
2-amino-N-((1R)-1-(3-fluoro-2-pyridinyl)ethyl)-3-methyl-N-((5-(methylsulfonyl)-2-pyridinyl)methyl)-6-quinolinecarboxamide,
7-amino-6-methyl-N-((1R)-1-(2-pyrimidinyl)ethyl)-N-((5-(trifluoromethyl)-2-pyridinyl)methyl) -1,8-naphthyridine-3 -carboxamide,
2-amino-3-methyl-N-((1R)-1-(2-pyrimidinyl)ethyl)-N-((5-(trifluoromethoxy)-2-pyridinyl)methyl)-6-quinolinecarboxamide,
2-amino-3-methyl-N-((1R)-1-(2-pyrimidinyl)ethyl)-N-((6-(trifluoromethyl)-3-pyridazinyl)methyl)-6-quinolinecarboxamide,
2-amino-N-((5-cyano-2-pyridinyl)methyl)-N-((1R)-1-(3-fluoro-2-pyridinyl)ethyl)-3-methyl-6-quinolinecarboxamide,
2-amino-N-((3-fluoro-2-pyridinyl)methyl)-3-methyl-N-(4-(trifluoromethyl)benzyl)-6-quinolinecarboxamide,
2-amino-N-((3-fluoro-2-pyridinyl)methyl)-3-methyl-N-(2-methyl-4-(trifluoromethyl)benzyl)-6-quinolinecarboxamide,
7-amino-6-bromo-N-((1R)-1-(2-pyrimidinyl)ethyl)-N-((5-(trifluoromethyl)-2-pyridinyl)methyl) -1,8-naphthyridine-3 -carboxamide,
2-amino-N-((5-(cyclopropylsulfonyl)-2-pyridinyl)methyl)-N-((1R)-1-(3-fluoro-2-pyridinyl)ethyl)-3-methyl-6-quinolinecarboxamide,
2-amino-N-((5-cyano-2-pyridinyl)methyl)-3-methyl-N-((1R)-1-(2-pyrimidinyl)ethyl)-6-quinolinecarboxamide,
2-amino-N-((5-bromo-2-pyridinyl)methyl)-N-((1R)-1-(3-chloro-2-pyridinyl)ethyl)-3-methyl-6-quinolinecarboxamide,
2-amino-N-((1R)-1-(5-chloro-2-pyridinyl)ethyl)-3-methyl-N-((5-(trifluoromethyl)-2-pyridinyl)methyl)-6-quinolinecarboxamide,
2-amino-3-methyl-N-((1R)-1-(2-pyrimidinyl)propyl)-N-((5-(trifluoromethyl)-2-pyridinyl)methyl)-6-quinolinecarboxamide,
2-amino-N-((5-chloro-2-pyridinyl)methyl)-N-((1R)-1-(3-fluoro-2-pyridinyl)ethyl)-3-methyl-6-quinolinecarboxamide,
2-amino-N-((5-chloro-2-pyridinyl)methyl)-3-methyl-N-(2-methylpropyl)-6-quinolinecarboxamide,
2-amino-N-((1R)-1-(4-fluorophenyl)ethyl)-3-methyl-N-((5-(trifluoromethyl)-2-pyridinyl)methyl)-6-quinolinecarboxamide,
7-amino-6-ethynyl-N-((1R)-1-(3-fluoro-2-pyridinyl)ethyl)-N-((5-(trifluoromethyl)-2-pyridinyl)methyl) -1,8-naphthyridine-3 -carboxamide,
2-amino-N-((5-bromo-2-pyridinyl)methyl)-3-methyl-N-((1R)-1-(2-pyrimidinyl)ethyl)-6-quinolinecarboxamide,
2-amino-N-((7R)-6,7-dihydro-5H-cyclopenta[b]pyridin-7-yl)-3-methyl-N-((6-(trifluoromethyl)-3-pyridazinyl)methyl)-6-quinolinecarboxamide,
2-amino-7-fluoro-N-((3-fluoro-2-pyridinyl)methyl)-3-methyl-N-((5-(trifluoromethyl)-2-pyridinyl)methyl)-6-quinolinecarboxamide,
7-amino-6-bromo-N-((1R)-1-(2-pyrimidinyl)ethyl)-N-((5-(trifluoromethyl)-2-pyrazinyl)methyl)-1,8-naphthyridine-3-carboxamide,
2-amino-3-methyl-N-(2-pyridinylmethyl)-N-((5-(trifluoromethyl)-2-pyridinyl)methyl)-6-quinolinecarboxamide,
methyl 6-((((2-amino-3-methyl-6-quinolinyl)carbonyl)((3-fluoro-2-pyridinyl)methyl)amino)methyl)-3-pyridinecarboxylate,
2-amino-N-((5-(difluoromethoxy)-2-pyridinyl)methyl)-3-methyl-N-((1R)-1-(2-pyrimidinyl)ethyl)-6-quinolinecarboxamide,
2-amino-N-((3-chloro-5-(trifluoromethyl)-2-pyridinyl)methyl)-N-((1R)-1-(3-fluoro-2-pyridinyl)ethyl)-3-methyl-6-quinolinecarboxamide,
2-amino-7-fluoro-3-methyl-N-((1R)-1-(2-pyrimidinyl)ethyl)-N-((5-(trifluoromethyl)-2-pyridinyl)methyl)-6-quinolinecarboxamide,
2-amino-N-(cyclopropylmethyl)-7-fluoro-3-methyl-N-((5-(trifluoromethyl)-2-pyridinyl)methyl)-6-quinolinecarboxamide,
methyl 6-((((2-amino-3-methyl-6-quinolinyl)carbonyl)((1R)-1-(3-fluoro-2-pyridinyl)ethyl)amino)methyl)-3-pyridinecarboxylate,
2-amino-N-((5-(dimethylcarbamoyl)-2-pyridinyl)methyl)-N-((3-fluoro-2-pyridinyl)methyl)-3-methyl-6-quinolinecarboxamide,
2-amino-3-methyl-N-(1-(2-pyrimidinyl)cyclopropyl)-N-((5-(trifluoromethyl)-2-pyridinyl)methyl)-6-quinolinecarboxamide,
2-amino-3-methyl-N-((1R)-1-(2-pyrimidinyl)ethyl)-N-((5-(trifluoromethyl)-1,3-thiazol-2-yl)methyl)-6-quinolinecarboxamide,
2-amino-3-methyl-N-(3-pyridinylmethyl)-N-((5-(trifluoromethyl)-2-pyridinyl)methyl)-6-quinolinecarboxamide,
2-amino-N-((2-amino-1,3-thiazol-5-yl)methyl)-3-methyl-N-((5-(trifluoromethyl)-2-pyridinyl)methyl)-6-quinolinecarboxamide,
2-amino-N-((5-cyano-2-pyridinyl)methyl)-N-((1R)-1-(3,5-difluoro-2-pyridinyl)ethyl)-3-methyl-6-quinolinecarboxamide,
2-amino-3-methyl-N-((5-((~2~H_3_)methyloxy)-2-pyridinyl)methyl)-N-((1R)-1-(2-pyrimidinyl)ethyl)-6-quinolinecarboxamide,
2-amino-N-(4-carbamoylbenzyl)-3-methyl-N-((5-(trifluoromethyl)-2-pyridinyl)methyl)-6-quinolinecarboxamide,
2-amino-N-((5-(difluoromethyl)-2-pyridinyl)methyl)-N-((1R)-1-(5-fluoro-2-pyrimidinyl)ethyl)-3-methyl-6-quinolinecarboxamide,
2-amino-N-((5-bromo-2-pyridinyl)methyl)-3-methyl-N-((3R)-1-methyl-2-oxo-3-piperidinyl)-6-quinolinecarboxamide,
2-amino-N-((5-(dimethylcarbamoyl)-2-pyridinyl)methyl)-N-((1R)-1-(3-fluoro-2-pyridinyl)ethyl)-3-methyl-6-quinolinecarboxamide,
2-amino-N-((5-cyano-2-pyridinyl)methyl)-3-methyl-N-((1R)-1-(2-pyridinyl)ethyl)-6-quinolinecarboxamide,
2-amino-3-methyl-N-((1R)-1-(2-pyrimidinyl)ethyl)-N-((5-(trifluoromethyl)-2-pyridinyl)methyl) -1, 7-naphthyridine-6-carboxamide,
2-amino-N-((3-fluoro-2-pyridinyl)methyl)-3-methyl-N-((5-(trifluoromethyl)-2-pyridinyl)methyl) -1, 7-naphthyridine-6-carboxamide,
2-amino-3-methyl-N-(2-methylpropyl)-N-((5-(trifluoromethyl)-2-pyridinyl)methyl)-1,7-naphthyridine-6-carboxamide,
2-amino-N-((5-cyano-2-pyridinyl)methyl)-N-((4R)-3,4-dihydro-2H-pyrano[3,2-b]pyridin-4-yl)-3-methyl-6-quinolinecarboxamide,
2-amino-N-((5-cyano-2-pyridinyl)methyl)-3-methyl-N-((1R)-1-(2-pyrimidinyl)propyl)-6-quinolinecarboxamide,
2-amino-3-methyl-N-(4-pyridinylmethyl)-N-((5-(trifluoromethyl)-2-pyridinyl)methyl)-6-quinolinecarboxamide,
2-amino-3-methyl-N-((3-methyl-2-pyridinyl)methyl)-N-((5-(trifluoromethyl)-2-pyridinyl)methyl)-6-quinolinecarboxamide,
2-amino-3-methyl-N-((4-methyl-1,3-thiazol-2-yl)methyl)-N-((5-(trifluoromethyl)-2-pyridinyl)methyl)-6-quinolinecarboxamide,
2-amino-3-methyl-N-(4-(1-piperidinyl)benzyl)-N-(1H-pyrrolo[2,3-b]pyridin-4-ylmethyl)-6-quinolinecarboxamide,
2-amino-N-(cyclobutylmethyl)-3-methyl-N-((5-(trifluoromethyl)-2-pyridinyl)methyl)-6-quinolinecarboxamide,
2-amino-3-methyl-N-(1,2,3-thiadiazol-4-ylmethyl)-N-((5-(trifluoromethyl)-2-pyridinyl)methyl)-6-quinolinecarboxamide,
2-amino-N-(1H-indol-3-ylmethyl)-3-methyl-N-((5-(trifluoromethyl)-2-pyridinyl)methyl)-6-quinolinecarboxamide,
2-amino-3-methyl-N-((5-methyl-1,3-oxazol-4-yl)methyl)-N-((5-(trifluoromethyl)-2-pyridinyl)methyl)-6-quinolinecarboxamide,
2-amino-4-methyl-N-((1-methyl-1H-pyrazol-3-yl)methyl)-N-((5-(trifluoromethyl)-2-pyridinyl)methyl)-6-quinolinecarboxamide,
2-amino-4-methyl-N-((1-methyl-1H-1,2,4-triazol-3-yl)methyl)-N-((5-(trifluoromethyl)-2-pyridinyl)methyl)-6-quinolinecarboxamide,
2-amino-N-(2-fluorobenzyl)-3-methyl-N-(1H-pyrrolo[3,2-b]pyridin-3-ylmethyl)-6-quinolinecarboxamide,
2-amino-3-methyl-N-((4-methyl-1,3-thiazol-5-yl)methyl)-N-((5-(trifluoromethyl)-2-pyridinyl)methyl)-6-quinolinecarboxamide,
2-amino-N-(3-fluoro-4-(hydroxymethyl)benzyl)-3-methyl-N-((5-(trifluoromethyl)-2-pyridinyl)methyl)-6-quinolinecarboxamide,
2-amino-N-((6-amino-3-pyridinyl)methyl)-3-methyl-N-((5-(trifluoromethyl)-2-pyridinyl)methyl)-6-quinolinecarboxamide,
2-amino-3-methyl-N-(1H-pyrrolo[2,3-b]pyridin-4-ylmethyl)-N-((5-(trifluoromethyl)-2-pyridinyl)methyl)-6-quinolinecarboxamide,
2-amino-N-(2-fluorobenzyl)-3-methyl-N-((3-oxo-3,4-dihydro-2H-1,4-benzoxazin-6-yl)methyl)-6-quinolinecarboxamide,
2-amino-3-methyl-N-((2-oxo-2,3-dihydro-1H-benzimidazol-5-yl)methyl)-N-((5-(trifluoromethyl)-2-pyridinyl)methyl)-6-quinolinecarboxamide,
2-amino-3-methyl-N-(1,3-oxazol-4-ylmethyl)-N-((5-(trifluoromethyl)-2-pyridinyl)methyl)-6-quinolinecarboxamide,
2-amino-3-methyl-N-((3R)-1-methyl-2-oxo-3-piperidinyl)-N-((5-(trifluoromethyl)-2-pyridinyl)methyl)-6-quinolinecarboxamide,
2-amino-4-methyl-N-((1R)-1-(2-pyrimidinyl)ethyl)-N-((5-(trifluoromethyl)-2-pyridinyl)methyl)-6-quinolinecarboxamide,
2-amino-N-((5-cyano-2-pyridinyl)methyl)-7-fluoro-3-methyl-N-((1R)-1-(2-pyrimidinyl)ethyl)-6-quinolinecarboxamide,
2-amino-N-((5-(difluoromethyl)-2-pyridinyl)methyl)-3-methyl-N-((1R)-1-(2-pyrimidinyl)ethyl)-6-quinolinecarboxamide,
2-amino-3-methyl-N-((3R)-2-oxo-3-piperidinyl)-N-((5-(trifluoromethyl)-2-pyridinyl)methyl)-6-quinolinecarboxamide,
2-amino-N-((5-chloro-2-pyridinyl)methyl)-3-methyl-N-((1R)-1-(2-pyrimidinyl)ethyl)-6-quinolinecarboxamide,
2-amino-3-methyl-N-((1R)-1-(2-pyrimidinyl)propyl)-N-((6-(trifluoromethyl)-3-pyridazinyl)methyl)-6-quinolinecarboxamide,
2-amino-4-chloro-N-((1R)-1-(2-pyrimidinyl)ethyl)-N-((5-(trifluoromethyl)-2-pyridinyl)methyl)-6-quinolinecarboxamide,
2-amino-N-((3R)-1-cyclopropyl-2-oxo-3-piperidinyl)-3-methyl-N-((5-(trifluoromethyl)-2-pyridinyl)methyl)-6-quinolinecarboxamide,
2-amino-3-methyl-N-((2-methyl-6-(trifluoromethyl)-3-pyridinyl)methyl)-N-((1R)-1-(2-pyrimidinyl)ethyl)-6-quinolinecarboxamide,
2-amino-N-((6-bromo-3-pyridazinyl)methyl)-3-methyl-N-((1R)-1-(2-pyrimidinyl)ethyl)-6-quinolinecarboxamide,
2-amino-N-((5-bromo-3-methyl-2-pyridinyl)methyl)-3-methyl-N-((1R)-1-(2-pyrimidinyl)ethyl)-6-quinolinecarboxamide,
2-amino-N-((3-chloro-2-pyridinyl)methyl)-3-methyl-N-((5-(trifluoromethyl)-2-pyridinyl)methyl)-6-quinolinecarboxamide,
2-amino-N-((5-cyano-3-methyl-2-pyridinyl)methyl)-3-methyl-N-((1R)-1-(2-pyrimidinyl)ethyl)-6-quinolinecarboxamide,
2-amino-N-(4-cyanobenzyl)-3-methyl-N-((1R)-1-(2-pyrimidinyl)ethyl)-6-quinolinecarboxamide,
2-amino-N-((5-cyano-2-pyridinyl)methyl)-N-((1R)-1-(2-fluorophenyl)ethyl)-3-methyl-6-quinolinecarboxamide,
2-amino-N-((5-chloro-3-fluoro-2-pyridinyl)methyl)-3-methyl-N-((5-(trifluoromethyl)-2-pyridinyl)methyl)-6-quinolinecarboxamide,
2-amino-N-(2,6-difluorobenzyl)-3-methyl-N-((5-(trifluoromethyl)-2-pyridinyl)methyl)-6-quinolinecarboxamide,
2-amino-3-methyl-N-((1R)-1-(2-pyrimidinyl)propyl)-N-((5-(trifluoromethyl)-2-pyrazinyl)methyl)-6-quinolinecarboxamide,
2-amino-3-chloro-N-((1R)-1-(2-pyrimidinyl)ethyl)-N-((5-(trifluoromethyl)-2-pyridinyl)methyl)-6-quinolinecarboxamide,
2-amino-N-cyclopropyl-7-fluoro-3-methyl-N-((5-(trifluoromethyl)-2-pyridinyl)methyl)-6-quinolinecarboxamide,
7-amino-6-bromo-N-((5-cyano-2-pyridinyl)methyl)-N-((1R)-1-(2-pyrimidinyl)ethyl)-1,8-naphthyridine-3-carboxamide,
7-amino-6-bromo-N-((5-(difluoromethyl)-2-pyridinyl)methyl)-N-((1R)-1-(2-pyrimidinyl)ethyl)-1,8-naphthyridine-3-carboxamide,
7-amino-6-bromo-N-((1R)-1-(2-pyrimidinyl)ethyl)-N-((6-(trifluoromethyl)-3-pyridazinyl)methyl)-1,8-naphthyridine-3-carboxamide,
2-amino-7-chloro-3-methyl-N-(2-methylpropyl)-N-((5-(trifluoromethyl)-2-pyridinyl)methyl)-6-quinolinecarboxamide,
2-amino-N-((5-methoxy-2-pyrazinyl)methyl)-3-methyl-N-((1R)-1-(2-pyrimidinyl)ethyl)-6-quinolinecarboxamide,
2-amino-N-((3-fluoro-2-pyridinyl)methyl)-3-methyl-N-((5R)-2-(trifluoromethyl)-6,7-dihydro-5H-cyclopenta[b]pyridin-5-yl)-6-quinolinecarboxamide,
2-amino-N-((5-cyano-2-pyrazinyl)methyl)-3-methyl-N-((1R)-1-(2-pyrimidinyl)ethyl)-6-quinolinecarboxamide,
2-amino-N-((R)-cyclopropyl(2-pyrimidinyl)methyl)-3-methyl-N-((5-(trifluoromethyl)-2-pyridinyl)methyl)-6-quinolinecarboxamide,
2-amino-3-chloro-N-((5-cyano-2-pyridinyl)methyl)-N-((1R)-1-(2-pyrimidinyl)ethyl)-6-quinolinecarboxamide,
2-amino-N-((6-methoxy-3-pyridazinyl)methyl)-3-methyl-N-((1R)-1-(2-pyrimidinyl)ethyl)-6-quinolinecarboxamide,
7-amino-N-((5-(difluoromethyl)-2-pyridinyl)methyl)-6-methyl-N-((1R)-1-(2-pyrimidinyl)ethyl)-1,8-naphthyridine-3-carboxamide,
2-methyl-2-propanyl 6-((((2-amino-3-methyl-6-quinolinyl)carbonyl)((1R)-1-(2-pyrimidinyl)ethyl)amino)methyl)-3’,6′-dihydro[3,4′-bipyridine]-1′(2′H)-carboxylate,
2-amino-3-methyl-N-((1R)-1-(2-pyrimidinyl)ethyl)-N-((6-(trifluoromethyl)-3-pyridinyl)methyl)-6-quinolinecarboxamide,
2-amino-3,7-dimethyl-N-(2-methylpropyl)-N-((5-(trifluoromethyl)-2-pyridinyl)methyl)-6-quinolinecarboxamide,
2-amino-N-((1R)-1-(3-fluoro-2-pyridinyl)ethyl)-3-methyl-N-((6-(trifluoromethyl)-3-pyridazinyl)methyl)-6-quinolinecarboxamide,
7-amino-6-bromo-N-((1R)-1-(3-fluoro-2-pyridinyl)ethyl)-N-((6-(trifluoromethyl)-3-pyridaziny l)methyl)-1 ,8-naphthyridine-3-carboxamide,
N-((1′-acetyl-1’,2,3′,6′-tetrahydro[3,4′-bipyridin]-6-yl)methyl)-2-amino-3-methyl-N-((1R)-1-(2-pyrimidinyl)ethyl)-6-quinolinecarboxamide,
2-amino-N-((5-carbamoyl-2-pyridinyl)methyl)-N-((1R)-1-(2-fluorophenyl)ethyl)-3-methyl-6-quinolinecarboxamide,
7-amino-6-methyl-N-(1H-pyrrolo[2,3-b]pyridin-4-ylmethyl)-N-((5-(trifluoromethyl)-2-pyridinyl)methyl) -1,8-naphthyridine-3 -carboxamide,
2-amino-3-methyl-N-((1R)-1-(1-methyl-1H-1,2,4-triazol-3-yl)ethyl)-N-((5-(trifluoromethyl)-2-pyridinyl)methyl)-6-quinolinecarboxamide,
2-amino-N-(1H-indazol-5-ylmethyl)-3-methyl-N-((5-(trifluoromethyl)-2-pyridinyl)methyl)-6-quinolinecarboxamide,
2-amino-3-methyl-N-((8R)-5,6,7,8-tetrahydro[1,2,4]triazolo[1,5-a]pyridin-8-yl)-N-((5-(trifluoromethyl)-2-pyridinyl)methyl)-6-quinolinecarboxamide,
7-amino-6-bromo-N-((1R)-1-(2,4-difluorophenyl)ethyl)-N-((5-(trifluoromethyl)-2-pyridinyl)methyl) -1,8-naphthyridine-3 -carboxamide,
2-amino-3-methyl-N-((5-(2-oxa-6-azaspiro[3.3]heptan-6-yl)-2-pyridinyl)methyl)-N-((1R)-1-(2-pyrimidinyl)ethyl)-6-quinolinecarboxamide,
2-amino-3-(~2~H_3_)methyl-N-(2-methylpropyl)-N-((5-(trifluoromethyl)-2-pyridinyl)methyl)-6-quinolinecarboxamide,
2-amino-3-chloro-N-((1R)-1-(2-pyrimidinyl)ethyl)-N-((6-(trifluoromethyl)-3-pyridazinyl)methyl)-6-quinolinecarboxamide,
2-amino-3-methyl-N-((1R)-1-(2-pyrimidinyl)ethyl)-N-(2-quinolinylmethyl)-6-quinolinecarboxamide,
2-amino-3-methyl-N-((1R)-1-(2-pyrimidinyl)ethyl)-N-(3-quinolinylmethyl)-6-quinolinecarboxamide,
2-amino-3-bromo-N-((1R)-1-(2-pyrimidinyl)ethyl)-N-((5-(trifluoromethyl)-2-pyridinyl)methyl)-6-quinolinecarboxamide,
2-amino-N-((5-cyano-2-pyridinyl)methyl)-N-(cyclopropylmethyl)-7-fluoro-3-methyl-6-quinolinecarboxamide,
2-amino-N-((5-(3,6-dihydro-2H-pyran-4-yl)-2-pyridinyl)methyl)-3-methyl-N-((1R)-1-(2-pyrimidinyl)ethyl)-6-quinolinecarboxamide,
2-amino-3 -(~2~H_3_)methyl-N-((1R)-1-(2-pyrimidinyl)ethyl)-N-((5-(trifluoromethyl)-2-pyridinyl)methyl)-6-quinolinecarboxamide,
2-amino-N-((1R,2R)-2-hydroxycyclohexyl)-3-methyl-N-((5-(trifluoromethyl)-2-pyridinyl)methyl)-6-quinolinecarboxamide,
2-amino-N-((4-cyano-2-pyridinyl)methyl)-3-methyl-N-((1R)-1-(2-pyrimidinyl)ethyl)-6-quinolinecarboxamide,
2-amino-N-((5-(5,6-dihydro-2H-pyran-3-yl)-2-pyridinyl)methyl)-3-methyl-N-((1R)-1-(2-pyrimidinyl)ethyl)-6-quinolinecarboxamide,
2-amino-N-((6-bromo-3-pyridazinyl)methyl)-3-chloro-N-((1R)-1-(2-pyrimidinyl)ethyl)-6-quinolinecarboxamide,
2-amino-N-((5-fluoro-2-pyridinyl)methyl)-3-methyl-N-((5-(trifluoromethyl)-2-pyridinyl)methyl)-6-quinolinecarboxamide,
2-amino-7-fluoro-3-methyl-N-((1R)-1-(1-methyl-1H-1,2,4-triazol-3-yl)ethyl)-N-((5-(trifluoromethyl)-2-pyridinyl)methyl)-6-quinolinecarboxamide,
2-amino-7-fluoro-3-methyl-N-((1S)-1-(1-methyl-1H-1,2,4-triazol-3-yl)ethyl)-N-((5-(trifluoromethyl)-2-pyridinyl)methyl)-6-quinolinecarboxamide,
2-amino-3-methyl-N-(2,2,2-trifluoroethyl)-N-((5-(trifluoromethyl)-2-pyridinyl)methyl)-6-quinolinecarboxamide,
2-amino-N-((4R)-3,4-dihydro-2H-pyrano[3,2-b]pyridin-4-yl)-3-methyl-N-((6-(trifluoromethyl)-3-pyridazinyl)methyl)-6-quinolinecarboxamide,
2-amino-N-((5-cyano-2-pyridinyl)methyl)-3-methyl-N-((1R)-1-(2-pyrimidinyl)ethyl)-1,7-naphthyridine-6-carboxamide,
2-amino-3-bromo-N-((5-cyano-2-pyridinyl)methyl)-N-((1R)-1-(2-pyrimidinyl)ethyl)-6-quinolinecarboxamide,
2-amino-N-(2-methoxyethyl)-3-methyl-N-((5-(trifluoromethyl)-2-pyridinyl)methyl)-6-quinolinecarboxamide,
2-amino-N-((5-ethoxy-2-pyridinyl)methyl)-3-methyl-N-((1R)-1-(2-pyrimidinyl)ethyl)-6-quinolinecarboxamide,
2-amino-7-chloro-3-methyl-N-((1R)-1-(2-pyrimidinyl)ethyl)-N-((5-(trifluoromethyl)-2-pyridinyl)methyl)-6-quinolinecarboxamide,
2-amino-N-((1R)-2-methoxy-1-(2-pyridinyl)ethyl)-3-methyl-N-((5-(trifluoromethyl)-2-pyridinyl)methyl)-6-quinolinecarboxamide,
2-amino-N-((1S)-2-methoxy-1-(2-pyridinyl)ethyl)-3-methyl-N-((5-(trifluoromethyl)-2-pyridinyl)methyl)-6-quinolinecarboxamide,
2-amino-N-((5-cyano-2-pyridinyl)methyl)-3,4-dimethyl-N-((1R)-1-(2-pyrimidinyl)ethyl)-6-quinolinecarboxamide,
2-amino-N-((5-cyano-2-pyridinyl)methyl)-3,4-dimethyl-N-((1S)-1-(2-pyrimidinyl)ethyl)-6-quinolinecarboxamide,
2-amino-N-((5-chloro-3-pyridinyl)methyl)-3-methyl-N-((5-(trifluoromethyl)-2-pyridinyl)methyl)-6-quinolinecarboxamide,
2-amino-N-(1H-indazol-4-ylmethyl)-3-methyl-N-((5-(trifluoromethyl)-2-pyridinyl)methyl)-6-quinolinecarboxamide,
7-amino-6-bromo-N-((1R)-1-(2-pyrimidinyl)propyl)-N-((5-(trifluoromethyl)-2-pyridinyl)methyl) -1,8-naphthyridine-3 -carboxamide,
2-amino-N-((6-bromo-3-pyridazinyl)methyl)-7-fluoro-3-methyl-N-((1R)-1-(2-pyrimidinyl)ethyl)-6-quinolinecarboxamide,
2-amino-3-methyl-N-((2-methyl-1H-pyrrolo[2,3-b]pyridin-4-yl)methyl)-N-((5 -(trifluoromethyl)-2-pyridinyl)methyl)-6-quinolinecarboxamide,
2-amino-3-bromo-N-((1R)-1-(2-pyrimidinyl)propyl)-N-((5-(trifluoromethyl)-2-pyridinyl)methyl) -1, 7-naphthyridine-6-carboxamide,
7-amino-6-bromo-N-((5-(difluoromethyl)-2-pyridinyl)methyl)-N-((1R)-1-(2-pyrimidinyl)propyl)-1,8-naphthyridine-3-carboxamide,
2-amino-N-((5-(cyclopropyloxy)-2-pyridinyl)methyl)-3-methyl-N-((1R)-1-(2-pyrimidinyl)ethyl)-6-quinolinecarboxamide,
7-amino-6-bromo-N-((6-bromo-3-pyridazinyl)methyl)-N-((1R)-1-(2-pyrimidinyl)ethyl)-1,8-naphthyridine-3 -carboxamide,
2-amino-N-((1R)-1-(3-fluoro-2-pyridinyl)ethyl)-3-(~2~H_3_)methyl-N-((5-(trifluoromethyl)-2-pyridinyl)methyl)-6-quinolinecarboxamide,
2-amino-N-((5-(3,3-difluoro-1-pyrrolidinyl)-2-pyridinyl)methyl)-3-methyl-N-((1R)-1-(2-pyrimidinyl)ethyl)-6-quinolinecarboxamide,
2-amino-N-((2R)-1-methoxy-2-propanyl)-3-methyl-N-((5-(trifluoromethyl)-2-pyridinyl)methyl)-6-quinolinecarboxamide,
7-amino-6-bromo-N-((6-methoxy-3-pyridazinyl)methyl)-N-((1R)-1-(2-pyrimidinyl)ethyl)-1,8-naphthyridine-3-carboxamide,
2-amino-7-fluoro-N-((6-methoxy-3-pyridazinyl)methyl)-3-methyl-N-((1R)-1-(2-pyrimidinyl)ethyl)-6-quinolinecarboxamide,
2-amino-7-fluoro-N-((6-methoxy-3-pyridazinyl)methyl)-3-methyl-N-((1S)-1-(2-pyrimidinyl)ethyl)-6-quinolinecarboxamide,
2-amino-3-bromo-N-((5-(difluoromethyl)-2-pyridinyl)methyl)-N-((1R)-1-(2-pyrimidinyl)ethyl)-6-quinolinecarboxamide,
2-amino-3-bromo-N-((6-methoxy-3-pyridazinyl)methyl)-N-((1R)-1-(2-pyrimidinyl)ethyl)-6-quinolinecarboxamide,
2-amino-N-((5-cyano-2-pyridinyl)methyl)-3-(~2~H_3_)methyl-N-((1R)-1-(2-pyrimidinyl)ethyl)-6-quinolinecarboxamide,
2-amino-N-((5-bromo-6-methyl-2-pyridinyl)methyl)-3-methyl-N-((1R)-1-(2-pyrimidinyl)ethyl)-6-quinolinecarboxamide,
2-amino-3-bromo-N-((1R)-1-(2-pyrimidinyl)ethyl)-N-((5-(trifluoromethyl)-2-pyridinyl)methyl) -1, 7-naphthyridine-6-carboxamide,
7-amino-N-((R)-cyclopropyl(2-pyrimidinyl)methyl)-6-methyl-N-((5-(trifluoromethyl)-2-pyridinyl)methyl) -1,8-naphthyridine-3 -carboxamide,
2-amino-N-((6-(dimethylamino)-3-pyridazinyl)methyl)-3-methyl-N-((1R)-1-(2-pyrimidinyl)ethyl)-6-quinolinecarboxamide,
2-amino-N-((5-cyano-2-pyridinyl)methyl)-3-methyl-N-((1R)-1-(1-methyl-1H-1,2,4-triazol-3 -y l)ethyl)-6-quinolinecarboxamide,
2-amino-3-bromo-N-((5-(difluoromethyl)-2-pyridinyl)methyl)-N-((1R)-1-(2-pyrimidinyl)propyl)-6-quinolinecarboxamide,
2-amino-3-methyl-N-((6-(4-morpholinyl)-3-pyridazinyl)methyl)-N-((1R)-1-(2-pyrimidinyl)ethyl)-6-quinolinecarboxamide,
2-amino-N-((6-ethoxy-3-pyridazinyl)methyl)-3-methyl-N-(1R)-1-(2-pyrimidinyl)ethyl)-6-quinolinecarboxamide,
methyl 6-((((2-amino-3-methyl-6-quinolinyl)carbonyl)((1R)-1-(2-pyrimidinyl)ethyl)amino)methyl)-3’,6′-dihydro[3,4′-bipyridine]-1′(2’H)-carboxylate,
2-amino-N-((1R,2S)-3,3-difluoro-2-hydroxycyclohexyl)-3-methyl-N-((5-(trifluoromethyl)-2-pyridinyl)methyl)-6-quinolinecarboxamide,
2-amino-N-((5-(3,3-difluoro-1-azetidinyl)-2-pyridinyl)methyl)-3-methyl-N-((1R)-1-(2-pyrimidinyl)ethyl)-6-quinolinecarboxamide,
7-amino-6-bromo-N-((R)-cyclopropyl(2-pyrimidinyl)methyl)-N-((5-(trifluoromethyl)-2-pyridinyl)methyl) -1,8-naphthyridine-3 -carboxamide,
7-amino-6-bromo-N-((2S)-3,3,3-trifluoro-2-methoxypropyl)-N-((5-(trifluoromethyl)-2-pyridinyl)methyl) -1,8-naphthyridine-3 -carboxamide,
2-amino-N-((5-(difluoromethyl)-2-pyridinyl)methyl)-3-methyl-N-((1R)-1-(1-methyl-1H-1,2,4-triazol-3-yl)ethyl)-6-quinolinecarboxamide,
2-amino-N-(1,3-dimethoxy-2-propanyl)-3-methyl-N-((5-(trifluoromethyl)-2-pyridinyl)methyl)-6-quinolinecarboxamide,
2-amino-N-((1R)-1-(5-fluoro-2-pyrimidinyl)ethyl)-3-methyl-N-((5-(trifluoromethyl)-2-pyridinyl)methyl)-6-quinolinecarboxamide,
2-amino-3-methyl-N-((1R)-1-(2-pyrimidinyl)ethyl)-N-((5-(tetrahydro-2H-pyran-4-yl)-2-pyridinyl)methyl)-6-quinolinecarboxamide,
2-amino-3-methyl-N-((5R)-5,6,7,8-tetrahydro-5-quinoxalinyl)-N-((5-(trifluoromethyl)-2-pyridinyl)methyl)-6-quinolinecarboxamide,
2-ammo-3-methyl-N-((5S)-5,6,7,8-tetrahydro-5-quinoxalinyl)-N-((5-(trifluoromethyl)-2-pyridinyl)methyl)-6-quinolinecarboxamide,
2-amino-3-methyl-N-((1R)-1-(2-pyrimidinyl)ethyl)-N-((6-((2,2,2-trifluoroethyl)amino)-3-pyridazinyl)methyl)-6-quinolinecarboxamide,
2-amino-N-((5-cyano-2-pyridinyl)methyl)-3-methyl-N-((8R)-5,6,7,8-tetrahydro [1,2,4]triazolo [1,5-a] pyridin-8-yl)-6-quinolinecarboxamide,
2-amino-N-((3-fluoro-2-pyridinyl)methyl)-3-methyl-N-((3S)-6-(trifluoromethyl)-2,3-dihydro-1-benzofuran-3-yl)-6-quinolinecarboxamide,
2-amino-3-bromo-N-((5-chloro-2-pyridinyl)methyl)-N-((1R)-1-(2-pyrimidinyl)ethyl)-6-quinolinecarboxamide,
2-amino-3-chloro-N-((6-methoxy-3-pyridazinyl)methyl)-N-((1R)-1-(2-pyrimidinyl)ethyl)-6-quinolinecarboxamide,
7-amino-6-bromo-N-((5-chloro-2-pyridinyl)methyl)-N-((1R)-1-(2-pyrimidinyl)ethyl)-1,8-naphthyridine-3-carboxamide,
2-amino-N-((5-chloro-2-pyridinyl)methyl)-7-fluoro-3-methyl-N-((1R)-1-(2-pyrimidinyl)ethyl)-6-quinolinecarboxamide,
2-amino-N-((1R)-1-(3-chloro-2-pyridinyl)ethyl)-3-methyl-N-((6-(trifluoromethyl)-3-pyridazinyl)methyl)-6-quinolinecarboxamide,
7-amino-6-chloro-N-((1R)-1-(2-pyrimidinyl)ethyl)-N-((5-(trifluoromethyl)-2-pyridinyl)methyl) -1,8-naphthyridine-3 -carboxamide,
2-amino-N-((1R,2R)-2-hydroxycyclopentyl)-3-methyl-N-((5-(trifluoromethyl)-2-pyridinyl)methyl)-6-quinolinecarboxamide,
2-amino-N-((1R,2R)-2-hydroxycyclopentyl)-3-methyl-N-((6-(trifluoromethyl)-3-pyridazinyl)methyl)-6-quinolinecarboxamide,
7-amino-6-chloro-N-((5-(difluoromethyl)-2-pyridinyl)methyl)-N-((1R)-1-(2-pyrimidinyl)ethyl)-1,8-naphthyridine-3-carboxamide,
2-amino-N-((R)-cyclopropyl(2-pyrimidinyl)methyl)-3-methyl-N-((6-(trifluoromethyl)-3-pyridazinyl)methyl)-6-quinolinecarboxamide,
2-amino-N-((5-cyano-2-pyridinyl)methyl)-N-((R)-cyclopropyl(2-pyrimidinyl)methyl)-7-fluoro-3-methyl-6-quinolinecarboxamide,
2-amino-3-chloro-N-((5-cyano-2-pyridinyl)methyl)-N-((1R)-1-(3-fluoro-2-pyridinyl)ethyl)-6-quinolinecarboxamide,
2-amino-3-bromo-N-((5-cyano-2-pyridinyl)methyl)-N-((1R)-1-(3-fluoro-2-pyridinyl)ethyl)-6-quinolinecarboxamide,
2-amino-N-((4R)-6-fluoro-3,4-dihydro-2H-pyrano[3,2-b]pyridin-4-yl)-3-methyl-N-((5-(trifluoromethyl)-2-pyridinyl)methyl)-6-quinolinecarboxamide,
2-amino-N-((5-cyano-2-pyridinyl)methyl)-7-fluoro-N-((1R)-1-(3-fluoro-2-pyridinyl)ethyl)-3-methyl-6-quinolinecarboxamide,
2-amino-N-((6-(3,6-dihydro-2H-pyran-4-yl)-3-pyridazinyl)methyl)-3-methyl-N-((1R)-1-(2-pyrimidinyl)ethyl)-6-quinolinecarboxamide,
2-amino-3-chloro-N-((6-(3,6-dihydro-2H-pyran-4-yl)-3-pyridazinyl)methyl)-N-((1R)-1-(2-pyrimidinyl)ethyl)-6-quinolinecarboxamide,
7-amino-6-bromo-N-((5-cyano-2-pyridinyl)methyl)-N-((1R)-1-(3-fluoro-2-pyridinyl)ethyl)-1,8-naphthyridine-3-carboxamide,
7-amino-N-((1R)-1-(5-fluoro-2-pyrimidinyl)ethyl)-6-methyl-N-((5-(trifluoromethyl)-2-pyridinyl)methyl)-1,8-naphthyridine-3-carboxamide,
2-amino-N-((6-bromo-3-pyridazinyl)methyl)-3-methyl-N-((1R)-1-(1,3-thiazol-2-yl)ethyl)-6-quinolinecarboxamide,
2-amino-3-methyl-N-((1R)-1-(1,3-thiazol-4-yl)ethyl)-N-((6-(trifluoromethyl)-3-pyridazinyl)methyl)-6-quinolinecarboxamide,
2-amino-7-chloro-N-((5-cyano-2-pyridinyl)methyl)-3-methyl-N-((1R)-1-(2-pyrimidinyl)ethyl)-6-quinolinecarboxamide,
2-amino-N-((6-bromo-3-pyridazinyl)methyl)-3-methyl-N-((8R)-5,6,7,8-tetrahydro [1,2,4]triazolo [1,5-a] pyridin-8-yl)-6-quinolinecarboxamide,
2-amino-3-methyl-N-((1R)-1-(2-pyrimidinyl)ethyl)-N-((6-(trifluoromethyl)-2-pyridinyl)methyl)-6-quinolinecarboxamide,
2-amino-N-((6-(difluoromethyl)-3-pyridazinyl)methyl)-3-methyl-N-((1R)-1-(2-pyrimidinyl)ethyl)-6-quinolinecarboxamide,
2-amino-N-((5-(difluoromethyl)-2-pyridinyl)methyl)-N-((1R,2R)-2-hydroxycyclopentyl)-3-methyl-6-quinolinecarboxamide,
2-amino-N-((5-cyano-2-pyridinyl)methyl)-N-((1R,2R)-2-hydroxycyclopentyl)-3-methyl-6-quinolinecarboxamide,
2-amino-N-((6-bromo-3-pyridazinyl)methyl)-N-((1R,2R)-2-hydroxycyclopentyl)-3-methyl-6-quinolinecarboxamide,
2-amino-N-((1R,2R)-2-hydroxycyclopentyl)-N-((6-methoxy-3-pyridazinyl)methyl)-3-methyl-6-quinolinecarboxamide,
2-amino-3-chloro-N-((1R)-1-(2-pyrimidinyl)ethyl)-N-((5-(trifluoromethyl)-2-pyridinyl)methyl) -1, 7-naphthyridine-6-carboxamide,
2-amino-N-((5-(difluoromethyl)-2-pyridinyl)methyl)-3-methyl-N-((1R)-1-(5-methyl-1,2,4-oxadiazol-3-yl)ethyl)-6-quinolinecarboxamide,
2-amino-3-bromo-N-((6-bromo-3-pyridazinyl)methyl)-N-((1R)-1-(1,3-thiazol-2-yl)ethyl)-6-quinolinecarboxamide,
2-amino-3-chloro-N-((5-(difluoromethyl)-2-pyridinyl)methyl)-N-((1R)-1-(2-pyrimidinyl)ethyl)-6-quinolinecarboxamide,
2-amino-3-iodo-N-((1R)-1-(2-pyrimidinyl)ethyl)-N-((5-(trifluoromethyl)-2-pyridinyl)methyl)-6-quinolinecarboxamide,
2-amino-N-((5-cyano-2-pyridinyl)methyl)-N-((R)-cyclopropyl(2-pyrimidinyl)methyl)-3-methyl-6-quinolinecarboxamide,
7-amino-6-bromo-N-((6-bromo-3-pyridazinyl)methyl)-N-((1R)-1-(3-fluoro-2-pyridinyl)ethyl)-1,8-naphthyridine-3-carboxamide,
7-amino-6-bromo-N-((5-cyano-2-pyridinyl)methyl)-N-((R)-cyclopropyl(2-pyrimidinyl)methyl)-1,8-naphthyridine-3-carboxamide,
7-amino-6-bromo-N-((5-(difluoromethyl)-2-pyridinyl)methyl)-N-((1R)-1-(1-methyl-1H-1,2,4-triazol-3 -yl)ethyl)-1,8-naphthyridine-3 -carboxamide,
2-amino-N-((6-bromo-3-pyridazinyl)methyl)-3-methyl-N-((1R)-1-(1-methyl-1H-1,2,4-triazol-3 -y l)ethyl)-6-quinolinecarboxamide,
2-amino-N-((4R)-6-fluoro-3,4-dihydro-2H-pyrano[3,2-b]pyridin-4-yl)-3-methyl-N-((6-(trifluoromethyl)-3-pyridazinyl)methyl)-6-quinolinecarboxamide,
N-((5-(1-acetyl-4-piperidinyl)-2-pyridinyl)methyl)-2-amino-3-methyl-N-((1R)-1-(2-pyrimidinyl)ethyl)-6-quinolinecarboxamide,
7-amino-6-bromo-N-((5-(3-oxetanyloxy)-2-pyridinyl)methyl)-N-((1R)-1-(2-pyrimidinyl)ethyl)-1,8-naphthyridine-3-carboxamide,
2-amino-3-methyl-N-((5-(3-oxetanyloxy)-2-pyridinyl)methyl)-N-(1R)-1-(2-pyrimidinyl)ethyl)-6-quinolinecarboxamide,
7-amino-6-bromo-N-((5R)-5,6,7,8-tetrahydro-5-quinoxalinyl)-N-((5-(trifluoromethyl)-2-pyridinyl)methyl) -1,8-naphthyridine-3 -carboxamide,
2-amino-N-((5-chloro-6-methyl-2-pyridinyl)methyl)-3-methyl-N-((1R)-1-(2-pyrimidinyl)ethyl)-6-quinolinecarboxamide,
2-amino-N-((2-methoxy-6-(trifluoromethyl)-3-pyridinyl)methyl)-3-methyl-N-((1R)-1-(2-pyrimidinyl)ethyl)-6-quinolinecarboxamide,
7-amino-6-bromo-N-((6-ethoxy-3-pyridazinyl)methyl)-N-((1R)-1-(2-pyrimidinyl)ethyl)-1,8-naphthyridine-3 -carboxamide,
2-amino-N-((3R,4S)-4-hydroxytetrahydro-3-furanyl)-3-methyl-N-((5-(trifluoromethyl)-2-pyridinyl)methyl)-6-quinolinecarboxamide,
7-amino-6-bromo-N-((5-bromo-6-methyl-2-pyridinyl)methyl)-N-((1R)-1-(2-pyrimidinyl)ethyl)-1,8-naphthyridine-3-carboxamide,
2-amino-N-((3R,4R)-4-hydroxytetrahydro-2H-pyran-3-yl)-3-methyl-N-((5-(trifluoromethyl)-2-pyridinyl)methyl)-6-quinolinecarboxamide,
2-amino-N-((1S)-1-(6-cyano-2-pyridinyl)ethyl)-3-methyl-N-((5-(trifluoromethyl)-2-pyridinyl)methyl)-6-quinolinecarboxamide,
2-amino-N-((6-methoxy-3-pyridazinyl)methyl)-3-methyl-N-((1S)-1-(1-methyl-1H-1,2,4-triazol-3 -y l)ethyl)-6-quinolinecarboxamide,
2-amino-N-((3S,4S)-4-methoxytetrahydro-2H-pyran-3-yl)-3-methyl-N-((5-(trifluoromethyl)-2-pyridinyl)methyl)-6-quinolinecarboxamide,
2-amino-N-((2R)-1-methoxy-2-propanyl)-N-((6-methoxy-3-pyridazinyl)methyl)-3-methyl-6-quinolinecarboxamide,
2-amino-3-methyl-N-((1R)-2-methyl-1-(2-pyrimidinyl)propyl)-N-((5-(trifluoromethyl)-2-pyridinyl)methyl)-6-quinolinecarboxamide,
2-amino-3-methyl-N-((1S)-2-methyl-1-(2-pyrimidinyl)propyl)-N-((5-(trifluoromethyl)-2-pyridinyl)methyl)-6-quinolinecarboxamide,
7-amino-6-iodo-N-((1R)-1-(2-pyrimidinyl)ethyl)-N-((5-(trifluoromethyl)-2-pyridinyl)methyl) -1,8-naphthyridine-3 -carboxamide,
2-amino-3-methyl-N-((1R)-1-(2-pyrimidinyl)ethyl)-N-((6-(2,2,2-trifluoroethoxy)-3-pyridazinyl)methyl)-6-quinolinecarboxamide,
methyl 4-(6-((((2-amino-3-methyl-6-quinolinyl)carbonyl)((1R)-1-(2-pyrimidinyl)ethyl)amino)methyl)-3-pyridinyl)-1-piperidinecarboxylate,
2-amino-3-methyl-N-((8R)-5,6,7,8-tetrahydroimidazo[1,2-a]pyridin-8-yl)-N-((6-(trifluoromethyl)-3-pyridazinyl)methyl)-6-quinolinecarboxamide,
2-amino-N-((1R,2R)-2-methoxy-2,3-dihydro-1H-inden-1-yl)-3-methyl-N-((5-(trifluoromethyl)-2-pyridinyl)methyl)-6-quinolinecarboxamide,
2-amino-N-((3-chloro-2-pyridinyl)methyl)-7-fluoro-3-methyl-N-((1R)-1-(2-pyrimidinyl)ethyl)-6-quinolinecarboxamide,
2-amino-N-((6-methoxy-3-pyridazinyl)methyl)-3-methyl-N-((2S)-3,3,3-trifluoro-2-methoxypropyl)-6-quinolinecarboxamide,
2-amino-3-bromo-N-((6-bromo-3-pyridazinyl)methyl)-N-((1R)-1-(2-pyrimidinyl)ethyl)-6-quinolinecarboxamide,
2-amino-N-((1R)-1-(2-fluoro-4-pyridinyl)ethyl)-3-methyl-N-((5-(trifluoromethyl)-2-pyridinyl)methyl)-6-quinolinecarboxamide,
2-amino-N-((1S)-1-(2-fluoro-4-pyridinyl)ethyl)-3-methyl-N-((5-(trifluoromethyl)-2-pyridinyl)methyl)-6-quinolinecarboxamide,
2-amino-3-bromo-N-((5-cyano-2-pyridinyl)methyl)-N-((2R)-1-methoxy-2-propanyl)-6-quinolinecarboxamide,
2-amino-N-((1S,2S)-2-methoxy-2,3-dihydro-1H-inden-1-yl)-3-methyl-N-((5-(trifluoromethyl)-2-pyridinyl)methyl)-6-quinolinecarboxamide,
2-amino-N-((R)-cyclopropyl(2-pyrimidinyl)methyl)-N-((6-methoxy-3-pyridazinyl)methyl)-3-methyl-6-quinolinecarboxamide,
2-amino-3-methyl-N-(2-(trifluoromethoxy)ethyl)-N-((5-(trifluoromethyl)-2-pyridinyl)methyl)-6-quinolinecarboxamide,
2-amino-N-((1R)-1-(2-pyrimidinyl)ethyl)-N-((5-(trifluoromethyl)-2-pyridinyl)methyl)-6-quinolinecarboxamide,
2-amino-N-((6-cyano-3-pyridazinyl)methyl)-N-((1R)-1-(3-fluoro-2-pyridinyl)ethyl)-3-methyl-6-quinolinecarboxamide,
2-amino-N-((1R,2R)-4,4-difluoro-2-hydroxycyclopentyl)-3-methyl-N-((5-(trifluoromethyl)-2-pyridinyl)methyl)-6-quinolinecarboxamide,
2-amino-3-bromo-N-((6-ethoxy-3-pyridazinyl)methyl)-N-((1R)-1-(2-pyrimidinyl)ethyl)-6-quinolinecarboxamide,
2-amino-3,5-dimethyl-N-((1R)-1-(2-pyrimidinyl)ethyl)-N-((5-(trifluoromethyl)-2-pyridinyl)methyl)-6-quinolinecarboxamide,
2-amino-N-((2R)-1-methoxy-2-propanyl)-3,5-dimethyl-N-((5-(trifluoromethyl)-2-pyridinyl)methyl)-6-quinolinecarboxamide,
2-amino-N-((3-chloro-2-pyridinyl)methyl)-3-methyl-N-((1R)-1-(2-pyrimidinyl)ethyl)-6-quinolinecarboxamide,
2-amino-N-((3-chloro-2-pyridinyl)methyl)-3-methyl-N-((1S)-1-(2-pyrimidinyl)ethyl)-6-quinolinecarboxamide,
2-amino-N-((5S)-5,8-dihydro-6H-pyrano[3,4-b]pyridin-5-yl)-3-methyl-N-((5-(trifluoromethyl)-2-pyridinyl)methyl)-6-quinolinecarboxamide,
N-((5-acetyl-2-pyridinyl)methyl)-2-amino-3-methyl-N-((1R)-1-(2-pyrimidinyl)ethyl)-6-quinolinecarboxamide,
2-amino-N-((5-(2-hydroxy-2-propanyl)-2-pyridinyl)methyl)-3-methyl-N-((1R)-1-(2-pyrimidinyl)ethyl)-6-quinolinecarboxamide,
2-amino-N-((1R)-1-(3-fluoro-2-pyridinyl)ethyl)-3-methyl-N-((6-(4-morpholinyl)-3-pyridazinyl)methyl)-6-quinolinecarboxamide,
7-amino-6-bromo-N-(1R)-1-(3-fluoro-2-pyridinyl)ethyl)-N-((6-methoxy-3-pyridazinyl)methyl)-1,8-naphthyridine-3-carboxamide,
2-amino-3-bromo-N-(1R)-1-(3-fluoro-2-pyridinyl)ethyl)-N-((6-methoxy-3-pyridazinyl)methyl)-6-quinolinecarboxamide,
2-amino-3-bromo-N-((6-methoxy-3-pyridazinyl)methyl)-N-((1R)-1-(1-methyl-1H-1,2,4-triazol-3 -yl)ethyl)-6-quinolinecarboxamide,
2-amino-7-chloro-N-((3-fluoro-2-pyridinyl)methyl)-3-methyl-N-((5-(trifluoromethyl)-2-pyridinyl)methyl)-6-quinolinecarboxamide,
2-amino-N-((3R,4S)-3-fluorotetrahydro-2H-pyran-4-yl)-3-methyl-N-((5-(trifluoromethyl)-2-pyridinyl)methyl)-6-quinolinecarboxamide,
7-amino-6-bromo-N-((1S)-1-(1,3-thiazol-4-yl)ethyl)-N-((6-(trifluoromethyl)-3-pyridazinyl)methyl)-1,8-naphthyridine-3-carboxamide,
2-amino-N-((6-methoxy-3-pyridinyl)methyl)-3-methyl-N-((1R)-1-(2-pyrimidinyl)ethyl)-6-quinolinecarboxamide,
2-amino-N-((5-cyclopropyl-2-pyridinyl)methyl)-3-methyl-N-((1R)-1-(2-pyrimidinyl)ethyl)-6-quinolinecarboxamide,
7-amino-6-bromo-N-((1R)-1-(3-fluoro-2-pyridinyl)ethyl)-N-((6-(4-morpholinyl)-3-pyridazinyl)methyl)-1,8-naphthyridine-3-carboxamide,
2-amino-N-((3R,4S)-3-hydroxytetrahydro-2H-pyran-4-yl)-3-methyl-N-((5-(trifluoromethyl)-2-pyridinyl)methyl)-6-quinolinecarboxamide,
2-amino-N-((6-bromo-3-pyridazinyl)methyl)-3-iodo-N-((1R)-1-(2-pyrimidinyl)ethyl)-6-quinolinecarboxamide,
2-amino-3-bromo-N-((1R)-1-(3-fluoro-2-pyridinyl)ethyl)-N-((6-(trifluoromethyl)-3-pyridazinyl)methyl)-6-quinolinecarboxamide,
2-amino-N-((3R,4S)-3-methoxytetrahydro-2H-pyran-4-yl)-3-methyl-N-((5-(trifluoromethyl)-2-pyridinyl)methyl)-6-quinolinecarboxamide,
2-amino-N-(3,4-dihydro-2H-pyrano[2,3-c]pyridin-6-ylmethyl)-3-methyl-N-((1R)-1-(2-pyrimidinyl)ethyl)-6-quinolinecarboxamide,
2-amino-N-(3,4-dihydro-2H-pyrano[2,3-c]pyridin-6-ylmethyl)-3-methyl-N-((1S)-1-(2-pyrimidinyl)ethyl)-6-quinolinecarboxamide,
2-amino-3-bromo-N-((5-cyano-2-pyridinyl)methyl)-N-((R)-cyclopropyl(2-pyrimidinyl)methyl)-6-quinolinecarboxamide,
2-amino-N-((6-(difluoromethoxy)-3-pyridazinyl)methyl)-3-methyl-N-((1R)-1-(2-pyrimidinyl)ethyl)-6-quinolinecarboxamide,
2-amino-N-((6-(difluoromethoxy)-3-pyridazinyl)methyl)-3-methyl-N-((1S)-1-(2-pyrimidinyl)ethyl)-6-quinolinecarboxamide,
2-amino-N-((6-bromo-3-pyridazinyl)methyl)-7-fluoro-3-methyl-N-((8R)-5,6,7,8-tetrahydro [1,2,4]triazolo [1,5-a] pyridin-8-yl)-6-quinolinecarboxamide,
2-amino-3-methyl-N-(1S)-1-(1,2,4-oxadiazol-3-yl)ethyl)-N-((5-(trifluoromethyl)-2-pyridinyl)methyl)-6-quinolinecarboxamide,
2-amino-3-methyl-N-((1R)-1-(2-pyrimidinyl)ethyl)-N-((6-(trifluoromethoxy)-3-pyridazinyl)methyl)-6-quinolinecarboxamide,
2-amino-3-methyl-N-((1S)-1-(2-pyrimidinyl)ethyl)-N-((6-(trifluoromethoxy)-3-pyridazinyl)methyl)-6-quinolinecarboxamide,
2-amino-N-(3-isoquinolinylmethyl)-3-methyl-N-((1R)-1-(2-pyrimidinyl)ethyl)-6-quinolinecarboxamide,
2-amino-N-(3-isoquinolinylmethyl)-3-methyl-N-((1S)-1-(2-pyrimidinyl)ethyl)-6-quinolinecarboxamide,
7-amino-6-bromo-N-((1R,2R)-2-methoxycyclohexyl)-N-((5-(trifluoromethyl)-2-pyridinyl)methyl)-1,8-naphthyridine-3-carboxamide,
7-amino-6-bromo-N-((1S,2S)-2-methoxycycloheayl)-N-((5-(trifluoromethyl)-2-pyridinyl)methyl)-1,8-naphthyridine-3-carboxamide,
2-amino-N-((6-bromo-3-pyridazinyl)methyl)-3-chloro-N-((1R)-1-(3-fluoro-2-pyridinyl)ethyl)-6-quinolinecarboxamide,
2-amino-3-bromo-N-((6-bromo-3-pyridazinyl)methyl)-N-((8R)-5,6,7,8-tetrahydro [1,2,4]triazolo [1,5-a] pyridin-8-yl)-6-quinolinecarboxamide,
7-amino-6-iodo-N-((1R)-1-(2-pyrimidinyl)propyl)-N-((5-(trifluoromethyl)-2-pyridinyl)methyl) -1,8-naphthyridine-3 -carboxamide,
2-amino-3-methyl-N-(4-(3-oxetanyl)benzyl)-N-((1R)-1-(2-pyrimidinyl)ethyl)-6-quinolinecarboxamide,
2-amino-3-methyl-N-(4-(3-oxetanyl)benzyl)-N-((1S)-1-(2-pyrimidinyl)ethyl)-6-quinolinecarboxamide,
2-amino-N-((1R)-1-cyclopropyl-2-methoxyethyl)-3-methyl-N-((5-(trifluoromethyl)-2-pyridinyl)methyl)-6-quinolinecarboxamide,
2-amino-N-((1S)-1-cyclopropyl-2-methoxyethyl)-3-methyl-N-((5-(trifluoromethyl)-2-pyridinyl)methyl)-6-quinolinecarboxamide,
2-amino-N-((3R,4R)-4-fluorotetrahydro-2H-pyran-3-yl)-3-methyl-N-((5-(trifluoromethyl)-2-pyridinyl)methyl)-6-quinolinecarboxamide,
2-amino-N-((1R,2S)-2-cyanocyclopentyl)-3-methyl-N-((5-(trifluoromethyl)-2-pyridinyl)methyl)-6-quinolinecarboxamide,
2-amino-N-((1S,2R)-2-cyanocyclopentyl)-3-methyl-N-((5-(trifluoromethyl)-2-pyridinyl)methyl)-6-quinolinecarboxamide,
2-amino-N-((1S,2S)-2-cyanocyclopentyl)-3-methyl-N-((5-(trifluoromethyl)-2-pyridinyl)methyl)-6-quinolinecarboxamide,
2-amino-N-((1R)-1-(6-fluoro-2-pyridinyl)ethyl)-3-methyl-N-((5-(trifluoromethyl)-2-pyridinyl)methyl)-6-quinolinecarboxamide,
7-amino-6-bromo-N-((1R)-1-(2-pyrimidinyl)ethyl)-N-((6-(2,2,2-trifluoroethoxy)-3-pyridazinyl)methyl)-1,8-naphthyridine-3-carboxamide,
2-amino-7-fluoro-3-methyl-N-((6-(4-morpholinyl)-3-pyridazinyl)methyl)-N-((1R)-1-(2-pyrimidinyl)ethyl)-6-quinolinecarboxamide,
7-amino-N-((5-chloro-2-pyridinyl)methyl)-6-iodo-N-((1R)-1-(2-pyrimidinyl)ethyl)-1,8-naphthyridine-3-carboxamide,
7-amino-6-bromo-N-((1R)-1-(6-fluoro-2-pyridinyl)ethyl)-N-((5-(trifluoromethyl)-2-pyridinyl)methyl)-1,8-naphthyridine-3-carboxamide,
2-amino-3-methyl-N-((5-(4-morpholinyl)-2-pyridinyl)methyl)-N-((1R)-1-(2-pyrimidinyl)ethyl)-6-quinolinecarboxamide,
2-amino-3-bromo-N-((5-cyano-2-pyridinyl)methyl)-N-((1R)-1-(2-pyrimidinyl)propyl)-6-quinolinecarboxamide,
2-amino-N-((6-cyclopropyl-3-pyridazinyl)methyl)-3-methyl-N-((1R)-1-(2-pyrimidinyl)ethyl)-6-quinolinecarboxamide,
2-amino-N-((1R,2R)-2-(difluoromethoxy)cyclopentyl)-3-methyl-N-((5-(trifluoromethyl)-2-pyridinyl)methyl)-6-quinolinecarboxamide,
2-amino-N-((1S,2S)-2-(difluoromethoxy)cyclopentyl)-3-methyl-N-((5-(trifluoromethyl)-2-pyridinyl)methyl)-6-quinolinecarboxamide,
2-amino-N-((6-chloro-3-pyridazinyl)methyl)-3-methyl-N-((1R)-1-(2-pyrimidinyl)ethyl)-6-quinolinecarboxamide,
2-amino-N-((6-cyano-2-methyl-3-pyridinyl)methyl)-3-methyl-N-((1R)-1-(2-pyrimidinyl)ethyl)-6-quinolinecarboxamide,
2-amino-N-(2-methylpropyl)-3-phenyl-N-((5-(trifluoromethyl)-2-pyridinyl)methyl)-6-quinolinecarboxamide,
7-amino-6-bromo-5-methyl-N-((1R)-1-(2-pyrimidinyl)ethyl)-N-((5-(trifluoromethyl)-2-pyridinyl)methyl) -1,8-naphthyridine-3 -carboxamide,
2-amino-N-((5-(difluoromethyl)-2-pyridinyl)methyl)-3-iodo-N-((1R)-1-(1-methyl-1H-1,2,4-triazol-3-yl)ethyl)-6-quinolinecarboxamide,
2-amino-3-bromo-N-((6-cyclopropyl-3-pyridazinyl)methyl)-N-((1R)-1-(2-pyrimidinyl)ethyl)-6-quinolinecarboxamide,
2-amino-N-((4-chloro-5-cyano-2-pyridinyl)methyl)-3-methyl-N-((1R)-1-(2-pyrimidinyl)ethyl)-6-quinolinecarboxamide,
2-amino-N-((1R)-1-(3-fluoro-2-pyridinyl)ethyl)-3-methyl-N-((6-(methylamino)-3-pyridazinyl)methyl)-6-quinolinecarboxamide,
7-amino-6-bromo-N-((5-cyano-2-pyridinyl)methyl)-5-methyl-N-((1R)-1-(2-pyrimidinyl)ethyl)-1,8-naphthyridine-3-carboxamide,
2-amino-3-bromo-N-(1S)-1-cyclopropyl-2-methoxyethyl)-N-((6-(4-morpholinyl)-3-pyridazinyl)methyl)-6-quinolinecarboxamide,
2-amino-3-bromo-N-((4-chloro-5-cyano-2-pyridinyl)methyl)-N-((1R)-1-(2-pyrimidinyl)ethyl)-6-quinolinecarboxamide,
2-amino-N-((5-cyano-6-methyl-2-pyridinyl)methyl)-3-methyl-N-((1R)-1-(2-pyrimidinyl)ethyl)-6-quinolinecarboxamide,
2-amino-3-bromo-N-((1S)-2-cyano-1-cyclopropylethyl)-N-((5-cyano-2-pyridinyl)methyl)-6-quinolinecarboxamide,
2-amino-N-((4-ethyl-1,3-thiazol-2-yl)methyl)-3-methyl-N-((5-(trifluoromethyl)-2-pyridinyl)methyl)-6-quinolinecarboxamide,
2-amino-3-methyl-N-(1R)-1-((3R)-tetrahydro-3-furanyl)ethyl)-N-((5-(trifluoromethyl)-2-pyridinyl)methyl)-6-quinolinecarboxamide,
2-amino-3-methyl-N-((1R)-1-((3S)-tetrahydro-3-furanyl)ethyl)-N-((5-(trifluoromethyl)-2-pyridinyl)methyl)-6-quinolinecarboxamide,
2-amino-3-methyl-N-((1S)-1-((3R)-tetrahydro-3-furanyl)ethyl)-N-((5-(trifluoromethyl)-2-pyridinyl)methyl)-6-quinolinecarboxamide,
2-amino-3-methyl-N-((1S)-1-((3S)-tetrahydro-3-furanyl)ethyl)-N-((5-(trifluoromethyl)-2-pyridinyl)methyl)-6-quinolinecarboxamide,
2-amino-3-bromo-N-(3,4-dihydro-2H-pyrano[2,3-c]pyridin-6-ylmethyl)-N-((1R)-1-(2-pyrimidinyl)ethyl)-6-quinolinecarboxamide,
2-amino-3-bromo-N-((6-chloro-3-pyridazinyl)methyl)-N-((1R)-1-(2-pyrimidinyl)ethyl)-6-quinolinecarboxamide,
2-amino-3-bromo-N-((5-cyano-6-methyl-2-pyridinyl)methyl)-N-((1R)-1-(2-pyrimidinyl)ethyl)-6-quinolinecarboxamide,
2-amino-N-((5-cyano-6-methyl-2-pyridinyl)methyl)-3-iodo-N-((1R)-1-(2-pyrimidinyl)ethyl)-6-quinolinecarboxamide,
2-amino-3-bromo-N-((6-(difluoromethoxy)-3-pyridazinyl)methyl)-N-((1R)-1-(2-pyrimidinyl)ethyl)-6-quinolinecarboxamide,
2-amino-3-bromo-N-((1R,2R)-2-cyanocyclopentyl)-N-((5-cyano-2-pyridinyl)methyl)-6-quinolinecarboxamide,
2-amino-3-bromo-N-((1R,2S)-2-cyanocyclopentyl)-N-((5-cyano-2-pyridinyl)methyl)-6-quinolinecarboxamide,
2-amino-3-bromo-N-((1S,2R)-2-cyanocyclopentyl)-N-((5-cyano-2-pyridinyl)methyl)-6-quinolinecarboxamide,
2-amino-3-bromo-N-((1S,2S)-2-cyanocyclopentyl)-N-((5-cyano-2-pyridinyl)methyl)-6-quinolinecarboxamide,
2-amino-3-bromo-N-((1-cyanocyclopropyl)methyl)-N-((5-(trifluoromethyl)-2-pyridinyl)methyl)-6-quinolinecarboxamide,
2-amino-3-bromo-N-((3S,4R)-3-methoxytetrahydro-2H-pyran-4-yl)-N-((5-(trifluoromethyl)-2-pyridinyl)methyl)-6-quinolinecarboxamide,
2-amino-3-bromo-N-((3R,4R)-3-methyltetrahydro-2H-pyran-4-yl)-N-((5-(trifluoromethyl)-2-pyridinyl)methyl)-6-quinolinecarboxamide,
2-amino-3-bromo-N-((3R,4S)-3-methyltetrahydro-2H-pyran-4-yl)-N-((5-(trifluoromethyl)-2-pyridinyl)methyl)-6-quinolinecarboxamide,
2-amino-3-bromo-N-((3S,4R)-3-methyltetrahydro-2H-pyran-4-yl)-N-((5-(trifluoromethyl)-2-pyridinyl)methyl)-6-quinolinecarboxamide,
2-amino-3-bromo-N-((3S,4S)-3-methyltetrahydro-2H-pyran-4-yl)-N-((5-(trifluoromethyl)-2-pyridinyl)methyl)-6-quinolinecarboxamide,
2-amino-3-bromo-N-((1R,2R)-2-methylcyclopentyl)-N-((5-(trifluoromethyl)-2-pyridinyl)methyl)-6-quinolinecarboxamide,
2-amino-3-bromo-N-((1R,2S)-2-methylcyclopentyl)-N-((5-(trifluoromethyl)-2-pyridinyl)methyl)-6-quinolinecarboxamide,
2-amino-3-bromo-N-((1S,2R)-2-methylcyclopentyl)-N-((5-(trifluoromethyl)-2-pyridinyl)methyl)-6-quinolinecarboxamide,
2-amino-3-bromo-N-((1S,2S)-2-methylcyclopentyl)-N-((5-(trifluoromethyl)-2-pyridinyl)methyl)-6-quinolinecarboxamide,
2-amino-3-bromo-N-((2R,3R)-2-cyclopropyltetrahydro-3-furanyl)-N-((5-(trifluoromethyl)-2-pyridinyl)methyl)-6-quinolinecarboxamide,
2-amino-3-bromo-N-((2R,3S)-2-cyclopropyltetrahydro-3-furanyl)-N-((5-(trifluoromethyl)-2-pyridinyl)methyl)-6-quinolinecarboxamide,
2-amino-3-bromo-N-((2S,3R)-2-cyclopropyltetrahydro-3-furanyl)-N-((5-(trifluoromethyl)-2-pyridinyl)methyl)-6-quinolinecarboxamide,
2-amino-3-bromo-N-((2S,3S)-2-cyclopropyltetrahydro-3-furanyl)-N-((5-(trifluoromethyl)-2-pyridinyl)methyl)-6-quinolinecarboxamide,
2-amino-3-bromo-N-((1S)-2-cyano-1-cyclopropylethyl)-N-((5-(trifluoromethyl)-2-pyridinyl)methyl)-6-quinolinecarboxamide,
2-amino-N-((6-chloro-5-cyano-2-pyridinyl)methyl)-3-iodo-N-((1R)-1-(2-pyrimidinyl)ethyl)-6-quinolinecarboxamide,
2-amino-N-((5-cyano-2-pyridinyl)methyl)-3-iodo-N-((1R)-1-(2-pyrimidinyl)propyl)-6-quinolinecarboxamide,
2-amino-N-(3,4-dihydro-2H-pyrano[2,3-c]pyridin-6-ylmethyl)-3-iodo-N-((1R)-1-(2-pyrimidinyl)ethyl)-6-quinolinecarboxamide,
2-amino-N-((1S)-2-cyano-1-cyclopropylethyl)-N-((5-cyano-2-pyridinyl)methyl)-3-iodo-6-quinolinecarboxamide,
2-amino-N-((6-cyclopropyl-3-pyridazinyl)methyl)-3-iodo-N-((1R)-1-(2-pyrimidinyl)ethyl)-6-quinolinecarboxamide,
2-amino-7-hydroxy-3-methyl-N-(2-methylpropyl)-N-((5-(trifluoromethyl)-2-pyridinyl)methyl)-6-quinolinecarboxamide,
7-amino-6-iodo-N-((1R)-1-(2-pyrimidinyl)ethyl)-N-((6-(trifluoromethyl)-3-pyridazinyl)methyl)-1,8-naphthyridine-3-carboxamide,
7-amino-N-((6-ethoxy-3-pyridazinyl)methyl)-6-iodo-N-((1R)-1-(2-pyrimidinyl)ethyl)-1,8-naphthyridine-3 -carboxamide,
2-amino-3-methyl-N-(((3R,4R)-3-methyltetrahydro-2H-pyran-4-yl)methyl)-N-((5-(trifluoromethyl)-2-pyridinyl)methyl)-6-quinolinecarboxamide,
2-amino-3-methyl-N-(((3R,4S)-3-methyltetrahydro-2H-pyran-4-yl)methyl)-N-((5-(trifluoromethyl)-2-pyridinyl)methyl)-6-quinolinecarboxamide,
2-amino-3-methyl-N-(((3S,4S)-3-methyltetrahydro-2H-pyran-4-yl)methyl)-N-((5-(trifluoromethyl)-2-pyridinyl)methyl)-6-quinolinecarboxamide,
2-amino-3-methyl-N-(((3S,4R)-3-methyltetrahydro-2H-pyran-4-yl)methyl)-N-((5-(trifluoromethyl)-2-pyridinyl)methyl)-6-quinolinecarboxamide,
2-amino-3-methyl-N-((4R)-4,5,6,7-tetrahydro-1-benzofuran-4-yl)-N-((5-(trifluoromethyl)-2-pyridinyl)methyl)-6-quinolinecarboxamide,
2-amino-3-methyl-N-((4S)-4,5,6,7-tetrahydro-1-benzofuran-4-yl)-N-((5-(trifluoromethyl)-2-pyridinyl)methyl)-6-quinolinecarboxamide,
2-amino-N-((4R)-2-chloro-5,6-dihydro-4H-cyclopenta[b]thiophen-4-yl)-3-methyl-N-((5-(trifluoromethyl)-2-pyridinyl)methyl)-6-quinolinecarboxamide,
2-amino-N-((4S)-2-chloro-5,6-dihydro-4H-cyclopenta[b]thiophen-4-yl)-3-methyl-N-((5-(trifluoromethyl)-2-pyridinyl)methyl)-6-quinolinecarboxamide,
2-amino-N-((3R)-2,3-dihydro-1-benzofuran-3-yl)-3-methyl-N-((5-(trifluoromethyl)-2-pyridinyl)methyl)-6-quinolinecarboxamide,
2-amino-N-((3S)-2,3-dihydro-1-benzofuran-3-yl)-3-methyl-N-((5-(trifluoromethyl)-2-pyridinyl)methyl)-6-quinolinecarboxamide,
2-amino-3-methyl-N-((2R)-3,3,3-trifluoro-2-methoxypropyl)-N-((5-(trifluoromethyl)-2-pyridinyl)methyl)-6-quinolinecarboxamide,
2-amino-N-((1R,3R)-3-hydroxycyclohexyl)-3-methyl-N-((5-(trifluoromethyl)-2-pyridinyl)methyl)-6-quinolinecarboxamide,
2-amino-N-((1R,3S)-3-hydroxycyclohexyl)-3-methyl-N-((5-(trifluoromethyl)-2-pyridinyl)methyl)-6-quinolinecarboxamide,
2-amino-N-((1S,3R)-3-hydroxycyclohexyl)-3-methyl-N-((5-(trifluoromethyl)-2-pyridinyl)methyl)-6-quinolinecarboxamide,
2-amino-N-((1S,3S)-3-hydroxycyclohexyl)-3-methyl-N-((5-(trifluoromethyl)-2-pyridinyl)methyl)-6-quinolinecarboxamide,
2-amino-N-((5-fluoro-3-pyridinyl)methyl)-3-methyl-N-((5-(trifluoromethyl)-2-pyridinyl)methyl)-6-quinolinecarboxamide,
2-amino-3-methyl-N-((5R)-4,5,6,7-tetrahydro-1H-benzimidazol-5-ylmethyl)-N-((5-(trifluoromethyl)-2-pyridinyl)methyl)-6-quinolinecarboxamide,
2-amino-3-methyl-N-((5S)-4,5,6,7-tetrahydro-1H-benzimidazol-5-ylmethyl)-N-((5-(trifluoromethyl)-2-pyridinyl)methyl)-6-quinolinecarboxamide,
2-amino-3-methyl-N-(1H-pyrrolo[2,3-b]pyridin-5-ylmethyl)-N-((5-(trifluoromethyl)-2-pyridinyl)methyl)-6-quinolinecarboxamide,
2-amino-3-methyl-N-((2-oxo-2,5,6,7-tetrahydro-1H-cyclopenta[b]pyridin-3-yl)methyl)-N-((5-(trifluoromethyl)-2-pyridinyl)methyl)-6-quinolinecarboxamide,
2-amino-N-(3-hydroxybenzyl)-3-methyl-N-((5-(trifluoromethyl)-2-pyridinyl)methyl)-6-quinolinecarboxamide,
2-amino-N-((1R)-2,3-dihydro-1H-inden-1-yl)-3-methyl-N-((5-(trifluoromethyl)-2-pyridinyl)methyl)-6-quinolinecarboxamide,
2-amino-3-methyl-N-((1R)-6-methyl-2,3-dihydro-1H-inden-1-yl)-N-((5-(trifluoromethyl)-2-pyridinyl)methyl)-6-quinolinecarboxamide,
2-amino-3-methyl-N-((1S)-6-methyl-2,3-dihydro-1H-inden-1-yl)-N-((5-(trifluoromethyl)-2-pyridinyl)methyl)-6-quinolinecarboxamide,
2-amino-N-(4-(4-carbamoyl-1-piperidinyl)benzyl)-N-((2R)-2-cyclopropylpropyl)-3-methyl-6-quinolinecarboxamide,
2-amino-N-(4-(4-carbamoyl-1-piperidinyl)benzyl)-N-((2S)-2-cyclopropylpropyl)-3-methyl-6-quinolinecarboxamide,
2-amino-N-((2R)-2-cyclopropylpropyl)-3-methyl-N-((5-(trifluoromethyl)-2-pyridinyl)methyl)-6-quinolinecarboxamide,
2-amino-N-((2S)-2-cyclopropylpropyl)-3-methyl-N-((5-(trifluoromethyl)-2-pyridinyl)methyl)-6-quinolinecarboxamide,
(6R)-2-amino-N-(1H-pyrrolo[2,3-b]pyridin-4-ylmethyl)-N-((5-(trifluoromethyl)-2-pyridinyl)methyl)-5,6, 7,8-tetrahydro-6-quinolinecarboxamide,
(6S)-2-amino-N-(1H-pyrrolo[2,3-b]pyridin-4-ylmethyl)-N-((5-(trifluoromethyl)-2-pyridinyl)methyl)-5,6, 7,8-tetrahydro-6-quinolinecarboxamide,
2-amino-N-((2R)-2-ethoxypropyl)-3-methyl-N-((5-(trifluoromethyl)-2-pyridinyl)methyl)-6-quinolinecarboxamide,
2-amino-N-((2S)-2-ethoxypropyl)-3-methyl-N-((5-(trifluoromethyl)-2-pyridinyl)methyl)-6-quinolinecarboxamide,
2-amino-N-((1,3-dimethyl-1H-indol-2-yl)methyl)-3-methyl-N-((5-(trifluoromethyl)-2-pyridinyl)methyl)-6-quinolinecarboxamide,
2-amino-N-(2-fluorobenzyl)-N-((5-fluoro-3-pyridinyl)methyl)-3-methyl-6-quinolinecarboxamide,
2-amino-3-methyl-N-((2R)-3,3,3-trifluoro-2-hydroxypropyl)-N-((5-(trifluoromethyl)-2-pyridinyl)methyl)-6-quinolinecarboxamide,
2-amino-3-methyl-N-((2S)-3,3,3-trifluoro-2-hydroxypropyl)-N-((5-(trifluoromethyl)-2-pyridinyl)methyl)-6-quinolinecarboxamide,
2-amino-3-methyl-N-((7S)-4,5,6,7-tetrahydro-1H-indazol-7-yl)-N-((5-(trifluoromethyl)-2-pyridinyl)methyl)-6-quinolinecarboxamide,
7-amino-N-((1R,2R)-2-cyanocyclopentyl)-6-methyl-N-((5-(trifluoromethyl)-2-pyridinyl)methyl) -1,8-naphthyridine-3 -carboxamide,
7-amino-N-((1R,2S)-2-cyanocyclopentyl)-6-methyl-N-((5-(trifluoromethyl)-2-pyridinyl)methyl) -1,8-naphthyridine-3 -carboxamide,
7-amino-N-((1S,2R)-2-cyanocyclopentyl)-6-methyl-N-((5-(trifluoromethyl)-2-pyridinyl)methyl) -1,8-naphthyridine-3 -carboxamide,
7-amino-N-((1S,2S)-2-cyanocyclopentyl)-6-methyl-N-((5-(trifluoromethyl)-2-pyridinyl)methyl) -1,8-naphthyridine-3 -carboxamide,
2-amino-N-((1R,2R)-2-cyanocyclopentyl)-3-methyl-N-((5-(trifluoromethyl)-2-pyridinyl)methyl) -1, 7-naphthyridine-6-carboxamide,
2-amino-N-((1R,2S)-2-cyanocyclopentyl)-3-methyl-N-((5-(trifluoromethyl)-2-pyridinyl)methyl) -1, 7-naphthyridine-6-carboxamide,
2-amino-N-((1S,2R)-2-cyanocyclopentyl)-3-methyl-N-((5-(trifluoromethyl)-2-pyridinyl)methyl) -1, 7-naphthyridine-6-carboxamide,
2-amino-N-((1S,2S)-2-cyanocyclopentyl)-3-methyl-N-((5-(trifluoromethyl)-2-pyridinyl)methyl) -1, 7-naphthyridine-6-carboxamide,
2-amino-N-((1R,2R)-2-cyanocyclopentyl)-7-fluoro-3-methyl-N-((5-(trifluoromethyl)-2-pyridinyl)methyl)-6-quinolinecarboxamide,
2-amino-N-((1R,2S)-2-cyanocyclopentyl)-7-fluoro-3-methyl-N-((5-(trifluoromethyl)-2-pyridinyl)methyl)-6-quinolinecarboxamide,
2-amino-N-((1S,2R)-2-cyanocyclopentyl)-7-fluoro-3-methyl-N-((5-(trifluoromethyl)-2-pyridinyl)methyl)-6-quinolinecarboxamide,
2-amino-N-((1S,2S)-2-cyanocyclopentyl)-7-fluoro-3-methyl-N-((5-(trifluoromethyl)-2-pyridinyl)methyl)-6-quinolinecarboxamide,
2-amino-N-((1R,2R)-2-cyanocyclopentyl)-N-((5-cyano-2-pyridinyl)methyl)-7-fluoro-3-methyl-6-quinolinecarboxamide,
2-amino-N-((1R,2S)-2-cyanocyclopentyl)-N-((5-cyano-2-pyridinyl)methyl)-7-fluoro-3-methyl-6-quinolinecarboxamide,
2-amino-N-((1S,2R)-2-cyanocyclopentyl)-N-((5-cyano-2-pyridinyl)methyl)-7-fluoro-3-methyl-6-quinolinecarboxamide,
2-amino-N-((1S,2S)-2-cyanocyclopentyl)-N-((5-cyano-2-pyridinyl)methyl)-7-fluoro-3-methyl-6-quinolinecarboxamide,
2-amino-N-((1R,2R)-[1,1′-bi(cyclopropyl)]-2-yl)-3-methyl-N-((5-(trifluoromethyl)-2-pyridinyl)methyl)-6-quinolinecarboxamide,
2-amino-N-((1R,2 S)-[1,1′-bi(cyclopropyl)]-2-yl)-3-methyl-N-((5-(trifluoromethyl)-2-pyridinyl)methyl)-6-quinolinecarboxamide,
2-amino-N-((1S,2R)-[1,1′-bi(cyclopropyl)]-2-yl)-3-methyl-N-((5-(trifluoromethyl)-2-pyridinyl)methyl)-6-quinolinecarboxamide,
2-amino-N-((1S,2S)-[1,1′-bi(cyclopropyl)]-2-yl)-3-methyl-N-((5-(trifluoromethyl)-2-pyridinyl)methyl)-6-quinolinecarboxamide,
2-amino-N-((1R)-2,2-dimethylcyclopropyl)-3-methyl-N-((5-(trifluoromethyl)-2-pyridinyl)methyl)-6-quinolinecarboxamide,
2-amino-N-((1S)-2,2-dimethylcyclopropyl)-3-methyl-N-((5-(trifluoromethyl)-2-pyridinyl)methyl)-6-quinolinecarboxamide,
2-amino-N-(4-(4-carbamoyl-1-piperidinyl)benzyl)-3-methyl-N-((4-methyl-1,3-thiazol-5-yl)methyl)-6-quinolinecarboxamide,
2-amino-N-(4-(4-carbamoyl-1-piperidinyl)benzyl)-3-methyl-N-(1H-pyrrolo [2,3-b]pyridin-4-ylmethyl)-6-quinolinecarboxamide,
2-amino-N-((1-cyanocyclopropyl)methyl)-3-methyl-N-(4-(1-piperidinyl)benzyl)-6-quinolinecarboxamide,
2-amino-3-methyl-N-(2-(1,2,4-oxadiazol-3-yl)ethyl)-N-((5-(trifluoromethyl)-2-pyridinyl)methyl)-6-quinolinecarboxamide,
2-amino-N-((1-cyanocyclopropyl)methyl)-3-methyl-N-((5-(trifkioromethyl)-2-pyridinyl)methyl)-6-quinolinecarboxamide,
2-amino-N-(2-cyclobutylethyl)-3-methyl-N-((5-(trifluoromethyl)-2-pyridinyl)methyl)-6-quinolinecarboxamide,
2-amino-N-((4-chloro-1-methyl-1H-pyrrol-2-yl)methyl)-3-methyl-N-((5-(trifluoromethyl)-2-pyridinyl)methyl)-6-quinolinecarboxamide,
2-amino-3-methyl-N-(1H-pyrrol-3-ylmethyl)-N-((5-(trifluoromethyl)-2-pyridinyl)methyl)-6-quinolinecarboxamide,
2-amino-3-methyl-N-(2-(1,2-oxazol-3-yl)ethyl)-N-((5-(trifluoromethyl)-2-pyridinyl)methyl)-6-quinolinecarboxamide,
2-amino-N-(2-fluorobenzyl)-3-methyl-N-(cis-3-(4-(trifluoromethoxy)phenyl)cyclobutyl)-6-quinolinecarboxamide,
2-amino-N-(2-fluorobenzyl)-3-methyl-N-(trans-3-(4-(trifluoromethoxy)phenyl)cyclobutyl)-6-quinolinecarboxamide,
2-amino-N-((3-fluoro-4-pyridinyl)methyl)-3-methyl-N-((2-oxo-2,3-dihydro-1H-benzimidazol-5-yl)methyl)-6-quinolinecarboxamide,
2-amino-3-methyl-N-((2-oxo-2,3-dihydro-1H-benzimidazol-5-yl)methyl)-N-(1H-pyrrolo[2,3-b]pyridin-4-ylmethyl)-6-quinolinecarboxamide,
2-amino-N-((4R)-3,4-dihydro-1H-2-benzopyran-4-yl)-3-methyl-N-((5-(trifluoromethyl)-2-pyridinyl)methyl)-6-quinolinecarboxamide,
2-amino-N-((4S)-3,4-dihydro-1H-2-benzopyran-4-yl)-3-methyl-N-((5-(trifluoromethyl)-2-pyridinyl)methyl)-6-quinolinecarboxamide,
2-amino-N-((1R)-3,3-dimethyl-2,3-dihydro-1H-inden-1-yl)-3-methyl-N-((5-(trifluoromethyl)-2-pyridinyl)methyl)-6-quinolinecarboxamide,
2-amino-N-((1S)-3,3-dimethyl-2,3-dihydro-1H-inden-1-yl)-3-methyl-N-((5-(trifluoromethyl)-2-pyridinyl)methyl)-6-quinolinecarboxamide,
2-amino-3-methyl-N-((1R,3R)-3-methyl-2,3-dihydro-1H-inden-1-yl)-N-((5-(trifluoromethyl)-2-pyridinyl)methyl)-6-quinolinecarboxamide,
2-amino-3-methyl-N-((1R,3S)-3-methyl-2,3-dihydro-1H-inden-1-yl)-N-((5-(trifluoromethyl)-2-pyridinyl)methyl)-6-quinolinecarboxamide,
2-amino-3-methyl-N-((1S,3R)-3-methyl-2,3-dihydro-1H-inden-1-yl)-N-((5-(trifluoromethyl)-2-pyridinyl)methyl)-6-quinolinecarboxamide,
2-amino-3-methyl-N-((1S,3S)-3-methyl-2,3-dihydro-1H-inden-1-yl)-N-((5-(trifluoromethyl)-2-pyridinyl)methyl)-6-quinolinecarboxamide,
2-amino-3-methyl-N-((1R,4R)-4-methyl-1,2,3,4-tetrahydro-1-naphthalenyl)-N-((5-(trifluoromethyl)-2-pyridinyl)methyl)-6-quinolinecarboxamide,
2-amino-3-methyl-N-((,4S)-4-methyl-1,2,3,4-tetrahydro-1-naphthalenyl)-N-((5-(trifluoromethyl)-2-pyridinyl)methyl)-6-quinolinecarboxamide,
2-amino-3-methyl-N-((1S,4R)-4-methyl-1,2,3,4-tetrahydro-1-naphthalenyl)-N-((5-(trifluoromethyl)-2-pyridinyl)methyl)-6-quinolinecarboxamide,
2-amino-3-methyl-N-((1S,4S)-4-methyl-1,2,3,4-tetrahydro-1-naphthalenyl)-N-((5-(trifluoromethyl)-2-pyridinyl)methyl)-6-quinolinecarboxamide,
2-amino-N-((4R)-8-methoxy-3,4-dihydro-2H-chromen-4-yl)-3-methyl-N-((5-(trifluoromethyl)-2-pyridinyl)methyl)-6-quinolinecarboxamide,
2-amino-N-((4S)-8-methoxy-3,4-dihydro-2H-chromen-4-yl)-3-methyl-N-((5-(trifluoromethyl)-2-pyridinyl)methyl)-6-quinolinecarboxamide,
2-amino-3-methyl-N-((4R)-4,5,6,7-tetrahydro-1H-indol-4-yl)-N-((5-(trifluoromethyl)-2-pyridinyl)methyl)-6-quinolinecarboxamide,
2-amino-3-methyl-N-((4S)-4,5,6,7-tetrahydro-1H-indol-4-yl)-N-((5-(trifluoromethyl)-2-pyridinyl)methyl)-6-quinolinecarboxamide,
2-amino-3-methyl-N-((7R)-2-methyl-4,5,6,7-tetrahydro-1,3-benzothiazol-7-yl)-N-((5-(trifluoromethyl)-2-pyridinyl)methyl)-6-quinolinecarboxamide,
2-amino-3-methyl-N-((7S)-2-methyl-4,5,6,7-tetrahydro-1,3-benzothiazol-7-yl)-N-((5-(trifluoromethyl)-2-pyridinyl)methyl)-6-quinolinecarboxamide,
2-amino-3-methyl-N-((4R)-2-methyl-4,5,6,7-tetrahydro-2H-indazol-4-yl)-N-((5-(trifluoromethyl)-2-pyridinyl)methyl)-6-quinolinecarboxamide,
2-amino-3-methyl-N-((4S)-2-methyl-4,5,6,7-tetrahydro-2H-indazol-4-yl)-N-((5-(trifluoromethyl)-2-pyridinyl)methyl)-6-quinolinecarboxamide,
2-amino-N-((1R,2R)-2-hydroxy-2,3-dihydro-1H-inden-1-yl)-3-methyl-N-((5-(trifluoromethyl)-2-pyridinyl)methyl)-6-quinolinecarboxamide,
2-amino-N-((3R)-6,7-dimethyl-2,3-dihydro-1-benzofuran-3-yl)-3-methyl-N-((5-(trifluoromethyl)-2-pyridinyl)methyl)-6-quinolinecarboxamide,
2-amino-N-((3S)-6,7-dimethyl-2,3-dihydro-1-benzofuran-3-yl)-3-methyl-N-((5-(trifluoromethyl)-2-pyridinyl)methyl)-6-quinolinecarboxamide,
2-amino-N-((2R)-2-methoxy-3-methylbutyl)-3-methyl-N-((5-(trifluoromethyl)-2-pyridinyl)methyl)-6-quinolinecarboxamide,
2-amino-N-((2S)-2-methoxy-3-methylbutyl)-3-methyl-N-((5-(trifluoromethyl)-2-pyridinyl)methyl)-6-quinolinecarboxamide,
2-amino-3-methyl-N-((7R)-5,6,7,8-tetrahydroimidazo[1,2-a]pyridin-7-ylmethyl)-N-((5-(trifluoromethyl)-2-pyridinyl)methyl)-6-quinolinecarboxamide,
2-amino-3-methyl-N-((7S5,6,7,8-tetrahydroimidazo[1,2-a]pyridin-7-ylmethyl)-N-((5-(trifluoromethyl)-2-pyridinyl)methyl)-6-quinolinecarboxamide,
2-amino-3-methyl-N-(1H-pyrrolo[2,3-b]pyridin-6-ylmethyl)-N-((5-(trifluoromethyl)-2-pyridinyl)methyl)-6-quinolinecarboxamide,
2-amino-N-((2-amino-1H-benzimidazol-5-yl)methyl)-3-methyl-N-((5-(trifluoromethyl)-2-pyridinyl)methyl)-6-quinolinecarboxamide,
2-amino-N-(1H-indazol-6-ylmethyl)-3-methyl-N-((5-(trifluoromethyl)-2-pyridinyl)methyl)-6-quinolinecarboxamide,
2-amino-3-methyl-N-((2-oxo-2,3-dihydro-1,3-benzoxazol-6-yl)methyl)-N-((5-(trifluoromethyl)-2-pyridinyl)methyl)-6-quinolinecarboxamide,
2-amino-N-(imidazo[1,2-a]pyridin-7-ylmethyl)-3-methyl-N-((5-(trifluoromethyl)-2-pyridinyl)methyl)-6-quinolinecarboxamide,
2-amino-N-(1-benzofuran-6-ylmethyl)-3-methyl-N-((5-(trifluoromethyl)-2-pyridinyl)methyl)-6-quinolinecarboxamide,
2-amino-N-(1H-indol-5-ylmethyl)-3-methyl-N-((5-(trifluoromethyl)-2-pyridinyl)methyl)-6-quinolinecarboxamide,
2-amino-3-methyl-N-((1R)-1-(3-oxo-3,4-dihydro-2H-1,4-benzoxazin-6-yl)ethyl)-N-((5-(trifluoromethyl)-2-pyridinyl)methyl)-6-quinolinecarboxamide,
2-amino-3-methyl-N-((1S)-1-(3-oxo-3,4-dihydro-2H-1,4-benzoxazin-6-yl)ethyl)-N-((5-(trifluoromethyl)-2-pyridinyl)methyl)-6-quinolinecarboxamide,
2-amino-3-methyl-N-(5-pyrimidinylmethyl)-N-((5-(trifluoromethyl)-2-pyridinyl)methyl)-6-quinolinecarboxamide,
2-amino-3-methyl-N-((2-oxo-1,2-dihydro-3-pyridinyl)methyl)-N-((5-(trifluoromethyl)-2-pyridinyl)methyl)-6-quinolinecarboxamide,
2-amino-N-((1R)-4-hydroxy-2,3-dihydro-1H-inden-1-yl)-3-methyl-N-((5-(trifluoromethyl)-2-pyridinyl)methyl)-6-quinolinecarboxamide,
2-amino-N-((1S)-4-hydroxy-2,3-dihydro-1H-inden-1-yl)-3-methyl-N-((5-(trifluoromethyl)-2-pyridinyl)methyl)-6-quinolinecarboxamide,
2-amino-3-methyl-N-((3aR,4R,7aR)-octahydro-1-benzofuran-4-yl)-N-((5-(trifluoromethyl)-2-pyridinyl)methyl)-6-quinolinecarboxamide,
2-amino-3-methyl-N-((3aR,4R,7aS)-octahydro-1-benzofuran-4-yl)-N-((5-(trifluoromethyl)-2-pyridinyl)methyl)-6-quinolinecarboxamide,
2-amino-3-methyl-N-((3aR,4S,7aR)-octahydro-1-benzofuran-4-yl)-N-((5-(trifluoromethyl)-2-pyridinyl)methyl)-6-quinolinecarboxamide,
2-amino-3-methyl-N-((3aR,4S,7aS)-octahydro-1-benzofuran-4-yl)-N-((5-(trifluoromethyl)-2-pyridinyl)methyl)-6-quinolinecarboxamide,
2-amino-3-methyl-N-((3aS,4R,7aR)-octahydro-1-benzofuran-4-yl)-N-((5-(trifluoromethyl)-2-pyridinyl)methyl)-6-quinolinecarboxamide,
2-amino-3-methyl-N-((3aS,4R,7aS)-octahydro-1-benzofuran-4-yl)-N-((5-(trifluoromethyl)-2-pyridinyl)methyl)-6-quinolinecarboxamide,
2-amino-3-methyl-N-((3aS,4S,7aR)-octahydro-1-benzofuran-4-yl)-N-((5-(trifluoromethyl)-2-pyridinyl)methyl)-6-quinolinecarboxamide,
2-amino-3-methyl-N-((3aS,4S,7aS)-octahydro-1-benzofuran-4-yl)-N-((5-(trifluoromethyl)-2-pyridinyl)methyl)-6-quinolinecarboxamide,
2-amino-3-methyl-N-((2R)-4-methyl-2-pentanyl)-N-((5-(trifluoromethyl)-2-pyridinyl)methyl)-6-quinolinecarboxamide,
2-amino-3-methyl-N-((2S)-4-methyl-2-pentanyl)-N-((5-(trifluoromethyl)-2-pyridinyl)methyl)-6-quinolinecarboxamide,
2-amino-3-methyl-N-((1-oxo-2,3-dihydro-1H-isoindol-5-yl)methyl)-N-((5-(trifluoromethyl)-2-pyridinyl)methyl)-6-quinolinecarboxamide,
2-amino-N-((6-carbamoyl-3-pyridinyl)methyl)-3-methyl-N-((5-(trifluoromethyl)-2-pyridinyl)methyl)-6-quinolinecarboxamide,
2-amino-3-methyl-N-(4-(methylcarbamoyl)benzyl)-N-((5-(trifluoromethyl)-2-pyridinyl)methyl)-6-quinolinecarboxamide,
2-amino-N-((4-chloro-2-pyridinyl)methyl)-3-methyl-N-(2-methylpropyl)-6-quinolinecarboxamide,
2-amino-N-((3-bromo-2-pyridinyl)methyl)-3-methyl-N-(2-methylpropyl)-6-quinolinecarboxamide,
2-amino-N-((2R,3R)-3-hydroxy-2-butanyl)-3-methyl-N-((5-(trifluoromethyl)-2-pyridinyl)methyl)-6-quinolinecarboxamide,
2-amino-N-((2R,3S)-3-hydroxy-2-butanyl)-3-methyl-N-((5-(trifluoromethyl)-2-pyridinyl)methyl)-6-quinolinecarboxamide,
2-amino-N-((2S,3R)-3-hydroxy-2-butanyl)-3-methyl-N-((5-(trifluoromethyl)-2-pyridinyl)methyl)-6-quinolinecarboxamide,
2-amino-N-((2S,3S)-3-hydroxy-2-butanyl)-3-methyl-N-((5-(trifluoromethyl)-2-pyridinyl)methyl)-6-quinolinecarboxamide,
2-amino-3-methyl-N-((2R)-3-methyl-2-butanyl)-N-((5-(trifluoromethyl)-2-pyridinyl)methyl)-6-quinolinecarboxamide,
2-amino-3-methyl-N-((2S)-3-methyl-2-butanyl)-N-((5-(trifluoromethyl)-2-pyridinyl)methyl)-6-quinolinecarboxamide,
2-amino-3-methyl-N-(2-methylpropyl)-N-(4-(1-pyrrolidinyl)benzyl)-6-quinolinecarboxamide,
2-amino-3-methyl-N-(2-methylpropyl)-N-(3-(1H-1,2,4-triazol-1-yl)benzyl)-6-quinolinecarboxamide,
2-amino-3-methyl-N-(4-(2-oxo-1-pyrrolidinyl)benzyl)-N-((5-(trifluoromethyl)-2-pyridinyl)methyl)-6-quinolinecarboxamide,
2-amino-3-methyl-N-(4-(1-pyrrolidinyl)benzyl)-N-((5-(trifluoromethyl)-2-pyridinyl)methyl)-6-quinolinecarboxamide,
2-amino-3-methyl-N-(4-(1H-pyrazol-3-yl)benzyl)-N-((5-(trifluoromethyl)-2-pyridinyl)methyl)-6-quinolinecarboxamide,
2-amino-N-(3-(1H-imidazol-1-yl)benzyl)-3-methyl-N-((5-(trifluoromethyl)-2-pyridinyl)methyl)-6-quinolinecarboxamide,
2-amino-3-methyl-N-(3-(1H-1,2,4-triazol-1-yl)benzyl)-N-((5-(trifluoromethyl)-2-pyridinyl)methyl)-6-quinolinecarboxamide,
2-amino-N-(3-(1H-imidazol-2-yl)benzyl)-3-methyl-N-((5-(trifluoromethyl)-2-pyridinyl)methyl)-6-quinolinecarboxamide,
2-amino-3-methyl-N-(3-(1H-1,2,3-triazol-1-yl)benzyl)-N-((5-(trifluoromethyl)-2-pyridinyl)methyl)-6-quinolinecarboxamide,
2-amino-3-methyl-N-((2-(1H-1,2,4-triazol-1-yl)-3-pyridinyl)methyl)-N-((5-(trifluoromethyl)-2-pyridinyl)methyl)-6-quinolinecarboxamide,
2-amino-N-(2-(1H-imidazol-1-yl)benzyl)-3-methyl-N-((5-(trifluoromethyl)-2-pyridinyl)methyl)-6-quinolinecarboxamide,
2-amino-3-methyl-N-(2-(1H-pyrazol-1-yl)benzyl)-N-((5-(trifluoromethyl)-2-pyridinyl)methyl)-6-quinolinecarboxamide,
2-amino-3-methyl-N-(2-propyn-1-yl)-N-((5-(trifluoromethyl)-2-pyridinyl)methyl)-6-quinolinecarboxamide,
2-amino-N-((2R)-3-butyn-2-yl)-3-methyl-N-((5-(trifluoromethyl)-2-pyridinyl)methyl)-6-quinolinecarboxamide,
2-amino-N-((2S)-3-butyn-2-yl)-3-methyl-N-((5-(trifluoromethyl)-2-pyridinyl)methyl)-6-quinolinecarboxamide,
2-amino-N-(2-butyn-1-yl)-3-methyl-N-((5-(trifluoromethyl)-2-pyridinyl)methyl)-6-quinolinecarboxamide,
2-amino-N-(4-hydroxy-2-butyn-1-yl)-3-methyl-N-((5-(trifluoromethyl)-2-pyridinyl)methyl)-6-quinolinecarboxamide,
2-amino-N-(3-butyn-1-yl)-3-methyl-N-((5-(trifluoromethyl)-2-pyridinyl)methyl)-6-quinolinecarboxamide,
2-amino-3-methyl-N-(4-((1R)-2,2,2-trifluoro-1-hydroxyethyl)benzyl)-N-((5-(trifluoromethyl)-2-pyridinyl)methyl)-6-quinolinecarboxamide,
2-amino-3-methyl-N-(4-((1S)-2,2,2-trifluoro-1-hydroxyethyl)benzyl)-N-((5-(trifluoromethyl)-2-pyridinyl)methyl)-6-quinolinecarboxamide,
2-amino-N-(4-hydroxybenzyl)-3-methyl-N-((5-(trifluoromethyl)-2-pyridinyl)methyl)-6-quinolinecarboxamide,
2-amino-N-(3-fluoro-4-hydroxybenzyl)-3-methyl-N-((5-(trifluoromethyl)-2-pyridinyl)methyl)-6-quinolinecarboxamide,
2-amino-N-((1R)-1-(3-hydroxyphenyl)ethyl)-3-methyl-N-((5-(trifluoromethyl)-2-pyridinyl)methyl)-6-quinolinecarboxamide,
2-amino-N-((1S)-1-(3-hydroayphenyl)ethyl)-3-methyl-N-((5-(trifluoromethyl)-2-pyridinyl)methyl)-6-quinolinecarboxamide,
2-amino-3-methyl-N-((1R)-1-(2-oxo-1,2-dihydro-4-pyridinyl)ethyl)-N-((5-(trifluoromethyl)-2-pyridinyl)methyl)-6-quinolinecarboxamide,
2-amino-3-methyl-N-((1 S)-1-(2-oxo-1,2-dihydro-4-pyridinyl)ethyl)-N-((5-(trifluoromethyl)-2-pyridinyl)methyl)-6-quinolinecarboxamide,
2-amino-N-((4-bromo-2-pyridinyl)methyl)-3-methyl-N-(2-methylpropyl)-6-quinolinecarboxamide,
2-amino-N-((8R)-4-methoxy-5,6,7,8-tetrahydro-8-quinolinyl)-3-methyl-N-((5-(trifluoromethyl)-2-pyridinyl)methyl)-6-quinolinecarboxamide,
2-amino-N-((8S)-4-methoxy-5,6,7,8-tetrahydro-8-quinolinyl)-3-methyl-N-((5-(trifluoromethyl)-2-pyridinyl)methyl)-6-quinolinecarboxamide,
2-amino-3-methyl-N-((5R)-5,6,7,8-tetrahydro-5-quinolinyl)-N-((5-(trifluoromethyl)-2-pyridinyl)methyl)-6-quinolinecarboxamide,
2-amino-3-methyl-N-((5S)-5,6,7,8-tetrahydro-5-quinolinyl)-N-((5-(trifluoromethyl)-2-pyridinyl)methyl)-6-quinolinecarboxamide,
2-amino-N-((6R)-2-amino-5,6,7,8-tetrahydro-6-quinazolinyl)-3-methyl-N-((5-(trifluoromethyl)-2-pyridinyl)methyl)-6-quinolinecarboxamide,
2-amino-N-((6S)-2-amino-5,6,7,8-tetrahydro-6-quinazolinyl)-3-methyl-N-((5-(trifluoromethyl)-2-pyridinyl)methyl)-6-quinolinecarboxamide,
2-amino-3-methyl-N-((3aR,5R,7aR)-octahydro-1H-inden-5-yl)-N-((5-(trifluoromethyl)-2-pyridinyl)methyl)-6-quinolinecarboxamide,
2-amino-3-methyl-N-((3aR,5R,7aS)-octahydro-1H-inden-5-yl)-N-((5-(trifluoromethyl)-2-pyridinyl)methyl)-6-quinolinecarboxamide,
2-amino-3-methyl-N-((3aR,5S,7aR)-octahydro-1H-inden-5-yl)-N-((5-(trifluoromethyl)-2-pyridinyl)methyl)-6-quinolinecarboxamide,
2-amino-3-methyl-N-((3aR,5S,7aS)-octahydro-1H-inden-5-yl)-N-((5-(trifluoromethyl)-2-pyridinyl)methyl)-6-quinolinecarboxamide,
2-amino-3-methyl-N-((3aS,5R,7aR)-octahydro-1H-inden-5-yl)-N-((5-(trifluoromethyl)-2-pyridinyl)methyl)-6-quinolinecarboxamide,
2-amino-3-methyl-N-((3aS,5R,7aS)-octahydro-1H-inden-5-yl)-N-((5-(trifluoromethyl)-2-pyridinyl)methyl)-6-quinolinecarboxamide,
2-amino-3-methyl-N-((3aS,5S,7aR)-octahydro-1H-inden-5-yl)-N-((5-(trifluoromethyl)-2-pyridinyl)methyl)-6-quinolinecarboxamide,
2-amino-3-methyl-N-((3aS,5S,7aS)-octahydro-1H-inden-5-yl)-N-((5-(trifluoromethyl)-2-pyridinyl)methyl)-6-quinolinecarboxamide,
2-amino-3-methyl-N-((5R)-4,5,6,7-tetrahydro-1H-benzimidazol-5-yl)-N-((5-(trifluoromethyl)-2-pyridinyl)methyl)-6-quinolinecarboxamide,
2-amino-3-methyl-N-((5S)-4,5,6,7-tetrahydro-1H-benzimidazol-5-yl)-N-((5-(trifluoromethyl)-2-pyridinyl)methyl)-6-quinolinecarboxamide,
2-amino-3-methyl-N-((6R)-4,5,6,7-tetrahydro-1H-indazol-6-yl)-N-((5-(trifluoromethyl)-2-pyridinyl)methyl)-6-quinolinecarboxamide,
2-amino-3-methyl-N-((6S)-4,5,6,7-tetrahydro-1H-indazol-6-yl)-N-((5-(trifluoromethyl)-2-pyridinyl)methyl)-6-quinolinecarboxamide,
2-amino-3-methyl-N-((5R)-4,5,6,7-tetrahydro-1H-indazol-5-yl)-N-((5-(trifluoromethyl)-2-pyridinyl)methyl)-6-quinolinecarboxamide,
2-amino-3-methyl-N-((5S)-4,5,6,7-tetrahydro-1H-indazol-5-yl)-N-((5-(trifluoromethyl)-2-pyridinyl)methyl)-6-quinolinecarboxamide,
2-amino-N-((4R)-1-ethyl-4,5,6,7-tetrahydro-1H-indazol-4-yl)-3-methyl-N-((5-(trifluoromethyl)-2-pyridinyl)methyl)-6-quinolinecarboxamide,
2-amino-N-((4S)-1-ethyl-4,5,6,7-tetrahydro-1H-indazol-4-yl)-3-methyl-N-((5-(trifluoromethyl)-2-pyridinyl)methyl)-6-quinolinecarboxamide,
2-amino-N-((1R,2R)-1-hydroxy-2,3-dihydro-1H-inden-2-yl)-3-methyl-N-((5-(trifluoromethyl)-2-pyridinyl)methyl)-6-quinolinecarboxamide,
2-amino-N-((1R,2S)-1-hydroxy-2,3-dihydro-1H-inden-2-yl)-3-methyl-N-((5-(trifluoromethyl)-2-pyridinyl)methyl)-6-quinolinecarboxamide,
2-amino-N-((1S,2R)-1-hydroxy-2,3-dihydro-1H-inden-2-yl)-3-methyl-N-((5-(trifluoromethyl)-2-pyridinyl)methyl)-6-quinolinecarboxamide,
2-amino-N-((1S,2S)-1-hydroxy-2,3-dihydro-1H-inden-2-yl)-3-methyl-N-((5-(trifluoromethyl)-2-pyridinyl)methyl)-6-quinolinecarboxamide,
2-amino-3-methyl-N-((1R)-1-(tetrahydro-2H-pyran-4-yl)ethyl)-N-((5-(trifluoromethyl)-2-pyridinyl)methyl)-6-quinolinecarboxamide,
2-amino-3-methyl-N-((1S)-1-(tetrahydro-2H-pyran-4-yl)ethyl)-N-((5-(trifluoromethyl)-2-pyridinyl)methyl)-6-quinolinecarboxamide,
2-amino-N-((trans-4-hydroxycyclohexyl)methyl)-3-methyl-N-((5-(trifluoromethyl)-2-pyridinyl)methyl)-6-quinolinecarboxamide,
2-amino-N-(((1R,3R)-3-hydroxycyclopentyl)methyl)-3-methyl-N-((5-(trifluoromethyl)-2-pyridinyl)methyl)-6-quinolinecarboxamide,
2-amino-N-(((1R,3S)-3-hydroxycyclopentyl)methyl)-3-methyl-N-((5-(trifluoromethyl)-2-pyridinyl)methyl)-6-quinolinecarboxamide,
2-amino-N-(((1S,3R)-3-hydroxycyclopentyl)methyl)-3-methyl-N-((5-(trifluoromethyl)-2-pyridinyl)methyl)-6-quinolinecarboxamide,
2-amino-N-(((1S,3S)-3-hydroxycyclopentyl)methyl)-3-methyl-N-((5-(trifluoromethyl)-2-pyridinyl)methyl)-6-quinolinecarboxamide,
2-amino-N-(((1R,3R)-3-hydroxycyclohexyl)methyl)-3-methyl-N-((5-(trifluoromethyl)-2-pyridinyl)methyl)-6-quinolinecarboxamide,
2-amino-N-(((1R,3S)-3-hydroxycyclohexyl)methyl)-3-methyl-N-((5-(trifluoromethyl)-2-pyridinyl)methyl)-6-quinolinecarboxamide,
2-amino-N-(((1S,3R)-3-hydroxycyclohexyl)methyl)-3-methyl-N-((5-(trifluoromethyl)-2-pyridinyl)methyl)-6-quinolinecarboxamide,
2-amino-N-(((1S,3S)-3-hydroxycyclohexyl)methyl)-3-methyl-N-((5-(trifluoromethyl)-2-pyridinyl)methyl)-6-quinolinecarboxamide,
2-amino-3-methyl-N-((2R,3R)-3-(1H-pyrazol-1-yl)-2-butanyl)-N-((5-(trifluoromethyl)-2-pyridinyl)methyl)-6-quinolinecarboxamide,
2-amino-3-methyl-N-((2R,3S)-3-(1H-pyrazol-1-yl)-2-butanyl)-N-((5-(trifluoromethyl)-2-pyridinyl)methyl)-6-quinolinecarboxamide,
2-amino-3-methyl-N-((2S,3R)-3-(1H-pyrazol-1-yl)-2-butanyl)-N-((5-(trifluoromethyl)-2-pyridinyl)methyl)-6-quinolinecarboxamide,
2-amino-3-methyl-N-((2S,3S)-3-(1H-pyrazol-1-yl)-2-butanyl)-N-((5-(trifluoromethyl)-2-pyridinyl)methyl)-6-quinolinecarboxamide,
2-amino-N-(4-(2-amino-2-oxoethyl)benzyl)-3-methyl-N-((5-(trifluoromethyl)-2-pyridinyl)methyl)-6-quinolinecarboxamide,
2-amino-3-methyl-N-((1R)-1-(tetrahydro-2H-pyran-4-yl)propyl)-N-((5-(trifluoromethyl)-2-pyridinyl)methyl)-6-quinolinecarboxamide,
2-amino-3-methyl-N-((1S)-1-(tetrahydro-2H-pyran-4-yl)propyl)-N-((5-(trifluoromethyl)-2-pyridinyl)methyl)-6-quinolinecarboxamide,
2-amino-N-((1R)-3-amino-3-oxo-1-(tetrahydro-2H-pyran-4-yl)propyl)-3-methyl-N-((5-(trifluoromethyl)-2-pyridinyl)methyl)-6-quinolinecarboxamide,
2-amino-N-((1S)-3-amino-3-oxo-1-(tetrahydro-2H-pyran-4-yl)propyl)-3-methyl-N-((5-(trifluoromethyl)-2-pyridinyl)methyl)-6-quinolinecarboxamide,
2-amino-3-methyl-N-(tetrahydro-2H-pyran-4-ylmethyl)-N-((5-(trifluoromethyl)-2-pyridinyl)methyl)-6-quinolinecarboxamide,
2-amino-3-methyl-N-(1H-pyrrolo[2,3-b]pyridin-4-ylmethyl)-N-((5-(trifluoromethyl)-2-pyridinyl)methyl)-1,7-naphthyridine-6-carboxamide,
2-amino-N-((3,5-difluoro-4-pyridinyl)methyl)-3-methyl-N-(4-(trifluoromethyl)benzyl)-6-quinolinecarboxamide,
2-amino-3-methyl-N-((1-methyl-1H-pyrazol-5-yl)methyl)-N-((5-(trifluoromethyl)-2-pyridinyl)methyl)-6-quinolinecarboxamide,
2-amino-3-methyl-N-((1-methyl-1H-pyrazol-5-yl)methyl)-N-(1H-pyrrolo[2,3-b]pyridin-4-ylmethyl)-6-quinolinecarboxamide,
2-amino-N-(4-hydroaybenzyl)-3-methyl-N-(1H-pyrrolo[2,3-b]pyridin-4-ylmethyl)-6-quinolinecarboxamide,
2-amino-3-methyl-N-(1H-pyrrolo[2,3-b]pyridin-4-ylmethyl)-N-(1H-pyrrol-2-ylmethyl)-6-quinolinecarboxamide,
2-amino-N-(2-hydroaybenzyl)-3-methyl-N-(1H-pyrrolo[2,3-b]pyridin-4-ylmethyl)-6-quinolinecarboxamide,
2-amino-N-((5-amino-2-pyridinyl)methyl)-N-(1H-indol-3-ylmethyl)-3-methyl-6-quinolinecarboxamide,
2-amino-3-methyl-N-(4-pentyn-1-yl)-N-((5-(trifluoromethyl)-2-pyridinyl)methyl)-6-quinolinecarboxamide,
2-amino-N-((2R)-1-cyano-2-propanyl)-3-methyl-N-((5-(trifluoromethyl)-2-pyridinyl)methyl)-6-quinolinecarboxamide,
2-amino-N-((2S)-1-cyano-2-propanyl)-3-methyl-N-((5-(trifluoromethyl)-2-pyridinyl)methyl)-6-quinolinecarboxamide,
2-amino-N-((2R)-2-cyanopropyl)-3-methyl-N-((5-(trifluoromethyl)-2-pyridinyl)methyl)-6-quinolinecarboxamide,
2-amino-N-((2S)-2-cyanopropyl)-3-methyl-N-((5-(trifluoromethyl)-2-pyridinyl)methyl)-6-quinolinecarboxamide,
2-amino-N-(2-cyclopropylethyl)-3-methyl-N-((5-(trifluoromethyl)-2-pyridinyl)methyl)-6-quinolinecarboxamide,
2-amino-N-((2R)-1-cyclopropyl-2-propanyl)-3-methyl-N-((5-(trifluoromethyl)-2-pyridinyl)methyl)-6-quinolinecarboxamide,
2-amino-N-((2S)-1-cyclopropyl-2-propanyl)-3-methyl-N-((5-(trifluoromethyl)-2-pyridinyl)methyl)-6-quinolinecarboxamide,
2-amino-N-(cis-3-cyanocyclobutyl)-3-methyl-N-((5-(trifluoromethyl)-2-pyridinyl)methyl)-6-quinolinecarboxamide,
2-amino-N-(trans-3-cyanocyclobutyl)-3-methyl-N-((5-(trifluoromethyl)-2-pyridinyl)methyl)-6-quinolinecarboxamide and
2-amino-N-(6,6-difluorospiro[3.3]heptan-2-yl)-3-methyl-N-((5-(trifluoromethyl)-2-pyridinyl)methyl)-6-quinolinecarboxamide.

20. A method of treating a cancer, the method comprising administering to a subject an effective amount of the compound of claim 1, the tautomer thereof, the stereoisomer thereof, or the pharmaceutically acceptable salt of any of the foregoing.

21. The method of claim 20, wherein the cancer is selected from ovarian, lung, lymphoid, glioblastoma, colon, melanoma, gastric, pancreatic or bladder cancer.

22. A method of treating a cancer, the method comprising administering to a subject an effective amount of the compound of claim 19, the tautomer thereof, the stereoisomer thereof, or the pharmaceutically acceptable salt of any of the foregoing.

23. The method of claim 22, wherein the cancer is selected from ovarian, lung, lymphoid, glioblastoma, colon, melanoma, gastric, pancreatic or bladder cancer.

24. A pharmaceutical composition, comprising the compound of claim 1, the tautomer thereof, the stereoisomer thereof, or the pharmaceutically acceptable salt of any of the foregoing or a pharmaceutically acceptable salt thereof, and at least one pharmaceutically acceptable excipient.

25. A pharmaceutical composition, comprising the compound of claim 19, the tautomer thereof, the stereoisomer thereof, or the pharmaceutically acceptable salt of any of the foregoing or a pharmaceutically acceptable salt thereof, and at least one pharmaceutically acceptable excipient.
